(19) United States
(12) Patent Application Publication
(10) Pub. No.: US 2025/0333424 A1
(43) Pub. Date: Oct. 30, 2025

(54) ANTIVIRAL COMPOUNDS

(71) Applicant: Gilead Sciences, Inc., Foster City, CA (US)

(72) Inventors: Mark J. Bartlett, Castro Valley, CA (US); Michael O. Clarke, Redwood City, CA (US); Jennifer L. Cosman Ellis, Foster City, CA (US); Isaac D. Falk, San Mateo, CA (US); Ana Z. Gonzalez Buenrostro, San Mateo, CA (US); Anna E. Hurtley, San Mateo, CA (US); Christopher Kwon, San Francisco, CA (US); David W. Lin, Berkeley, CA (US); Michael L. Mitchell, Castro Valley, CA (US); Hong Yang, Fremont, CA (US)

(21) Appl. No.: 19/067,656

(22) Filed: Feb. 28, 2025

Related U.S. Application Data
(60) Provisional application No. 63/560,148, filed on Mar. 1, 2024.
(60) Provisional application No. 63/560,405, filed on Mar. 1, 2024.

(51) Int. Cl. C07D 513/14; A61K 31/52; A61K 31/5365; A61K 31/537; A61K 31/5377; A61K 31/5383; A61K 31/542; A61K 45/06; A61P 31/18; C07D 471/14; C07D 473/18; C07D 487/04; C07D 487/14; C07D 491/048; C07D 495/04; C07D 498/04; C07D 498/14; C07D 498/22; C07D 513/04; C07D 519/00

(52) U.S. Cl. CPC C07D 513/14; A61K 31/52; A61K 31/5365; A61K 31/537; A61K 31/5377; A61K 31/5383; A61K 31/542; A61K 45/06; A61P 31/18; C07D 471/14; C07D 473/18; C07D 487/04; C07D 487/14; C07D 491/048; C07D 495/04; C07D 498/04; C07D 498/14; C07D 498/22; C07D 513/04; C07D 519/00

(57) ABSTRACT

This disclosure relates compounds disclosed herein and pharmaceutical salts thereof, compositions and formulations containing such compounds, and methods of using and making such compounds.

### CROSS REFERENCE TO RELATED APPLICATIONS

This application claims the benefit of U.S. Provisional Application No. 63/560,148, filed on Mar. 1, 2024, and U.S. Provisional Application No. 63/560,405, filed on Mar. 1, 2024, the entire contents of which is hereby incorporated by reference in its entirety.

### TECHNICAL FIELD

This disclosure relates antiviral compounds and pharmaceutical salts thereof, compositions and formulations containing such compounds, and methods of using and making such compounds.

### BACKGROUND

Human immunodeficiency virus-1 (HIV-1) infection is a major public health problem, with millions of people around the world dealing with the life-long consequences of HIV infection. While a number of successful treatments have been developed to suppress HIV replication, curing the disease remains a major challenge due to the establishment of viral reservoirs that evade clearance by the immune system. A key step in the HIV lifecycle is the production of the viral proteins contained in the gag-pol polyprotein. Inhibitors of such proteins are a mainstay of clinical antiretroviral therapy for the treatment of HIV or AIDS.

Certain small molecules bind to immature reverse transcriptase within the context of the gag-pol polyprotein and cause premature dimerization and protease activation. Active HIV protease is able to cleave a host inflammasome sensor in the cytoplasm and ultimately trigger an inflammatory form of cell death. These small molecule HIV protease activators (HPA) represent a selective mechanism by which HIV-infected cells can be cleared, and overall viral load reduced.

### SUMMARY

The present disclosure relates to compounds of Formula (I) or pharmaceutical salts thereof,

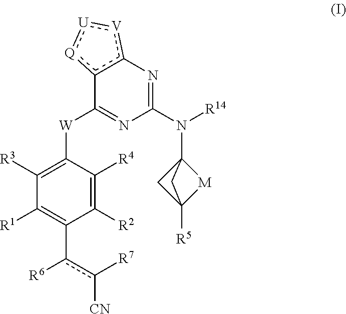

wherein [symbol] is a single bond or a double bond; and R¹, R², R³, R⁴, R⁵, R⁶, R⁷, R¹⁴, M, W, Q, U, and V are defined herein.

In certain embodiments, the current disclosure relates to a pharmaceutical composition comprising a compound of Formula (I), or a pharmaceutically acceptable salt thereof, and a pharmaceutically acceptable carrier.

In certain embodiments, the current disclosure relates to a method for treating or preventing an HIV infection in a subject in need thereof, comprising administering to the subject a compound of Formula (I), or a pharmaceutically acceptable salt thereof.

In certain embodiments, the current disclosure relates to a method for treating or preventing an HIV infection in a subject in need thereof, comprising administering to the subject a compound of Formula (I), or a pharmaceutically acceptable salt thereof, in combination with a therapeutically effective amount of one or more additional therapeutic agents.

Additional embodiments of the present disclosure are disclosed herein.

### DETAILED DESCRIPTION

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A wavy line drawn through a line in a chemical structure or a dashed line drawn through a line in a chemical structure indicates a point of attachment of a group. A dashed line within a chemical structure indicates an optional bond. A prefix such as “Cᵤ₋ᵥ” or (Cᵤ-Cᵥ) indicates that the following group has from u to v carbon atoms. For example, “C₁₋₆alkyl” indicates that the alkyl group has from 1 to 6 carbon atoms.

When trade names are used herein, it is intended to independently include the tradename product and the active pharmaceutical ingredient(s) of the tradename product.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

The term “about” used in connection with a quantity is inclusive of the stated value and has the meaning dictated by the context (e.g., includes the degree of error associated with measurement of the particular quantity).

The term “alkyl” is a straight or branched saturated hydrocarbon. For example, an alkyl group can have 1 to 8 carbon atoms (i.e., (C₁-C₅)alkyl) or 1 to 6 carbon atoms (i.e., (C₁-C₆ alkyl) or 1 to 4 carbon atoms (i.e., (C₁-C₄)alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—CH₂)₇CH₃).

The term “alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

The term “alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to, acetylenic (—C≡CH) and propargyl (—CH₂C≡CH).

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. It is also to be understood that when reference is made to a certain atom-range membered aryl (e.g., 6-12 membered aryl), the atom range is for the total ring atoms of the aryl. For example, a 6-membered aryl would include phenyl and a 10-membered aryl would include naphthyl and 1, 2, 3, 4-tetrahydronaphthyl. Non-limiting examples of aryl groups include, but are not limited to, phenyl, indenyl, naphthyl, 1, 2, 3, 4-tetrahydronaphthyl, anthracenyl, and the like.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

As used herein, the term “Cn-m alkoxy” refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

The term “chiral” refers to molecules which have the property of non-superimposability of the mirror image partner, and the term “achiral” refers to molecules which are superimposable on their mirror image partner.

The term “cycloalkyl” refers to a single saturated or partially unsaturated all carbon ring having 3 to 20 annular carbon atoms (i.e., C₃-C₂₀ cycloalkyl), for example from 3 to 12 annular atoms, for example from 3 to 10 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g., tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

As used herein, the term “effective amount” refers to an amount that is effective to elicit the desired biological or medical response, including the amount of a compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if in conjunction with one or more other agents, a desirable or beneficial result may be or is achieved. Suitable doses of any co-administered compounds may optionally be lowered due to the combined action (e.g., additive or synergistic effects) of the compounds.

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming an individual's HIV+ status and assessing the individual's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in individuals with functioning immune systems but does occur in AIDS patients). Development may also refer to disease progression that may be initially undetectable and includes occurrence, recurrence and onset.

The term “halo” or “halogen” as used herein refers to fluoro, chloro, bromo and iodo.

The term “haloalkyl” as used herein includes an alkyl group substituted with one or more halogens (e.g. F, Cl, Br, or I). Representative examples of haloalkyl include trifluoromethyl, 2,2,2-trifluoroethyl, and 2,2,2-trifluoro-1-(trifluoromethyl)ethyl.

The term “heteroaryl” as used herein refers to a single aromatic ring that has at least one atom other than carbon in the ring, wherein the atom is selected from the group consisting of oxygen, nitrogen and sulfur, the term also includes multiple condensed ring systems that have at least one such aromatic ring, which multiple condensed ring systems are further described below. Thus, the term includes single aromatic rings of from about 1 to 6 annular carbon atoms and about 1-4 annular heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycloalkyls, (to form for example a 1, 2, 3, 4-tetrahydronaphthyridinyl such as 1, 2, 3, 4-tetrahydro-1,8-naphthyridinyl), cycloalkyls (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 annular carbon atoms and about 1-6 annular heteroatoms. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl and thianaphthenyl.

“Heterocycloalkyl” or “heterocyclyl” as used herein refers to a single saturated or partially unsaturated non-aromatic ring or a non-aromatic multiple ring system that has at least one heteroatom in the ring (at least one annular heteroatom selected from oxygen, nitrogen, and sulfur). Unless otherwise specified, a heterocycloalkyl group has from 5 to about 20 annular atoms, for example from 5 to 14 annular atoms, for example from 5 to 10 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The term also includes single saturated or partially unsaturated rings (e.g., 5, 6, 7, 8, 9, or 10-membered rings) having from about 4 to 9 annular carbon atoms and from about 1 to 3 annular heteroatoms selected from the group consisting of oxygen, nitrogen and sulfur in the ring. The rings of the multiple condensed ring system can be connected to each other via fused, spiro and bridged bonds when allowed by valency requirements. Heterocycloalkyl groups include, but are not limited to, azetidine, aziridine, imidazolidine, imino-oxoimidazolidine, morpholine, oxirane (epoxide), oxetane, piperazine, piperidine, pyrazolidine, pyrrolidine, pyrrolidinone, tetrahydrofuran, tetrahydrothiophene, dihydropyridine, tetrahydropyridine, quinuclidine, and the like.

As used herein, the term “independently selected from” means that each occurrence of a variable or substituent is independently selected at each occurrence from the applicable list.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

The term “n-membered” where n is an integer typically describes the number of ring-forming atoms in a moiety where the number of ring-forming atoms is n. For example, pyridyl is an example of a 6-membered heteroaryl ring.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion (e.g. a sodium or potassium), an alkaline earth ion (e.g. calcium or magnesium), or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to an subject in the absence of detectable infectious agent (e.g., virus) in the subject). The subject may be an individual at risk of developing the disease or disorder, such as an individual who has one or more risk factors known to be associated with development or onset of the disease or disorder. Thus, the term “preventing HIV infection” refers to administering to a subject who does not have a detectable HIV infection an anti-HIV therapeutic substance. It is understood that the subject for anti-HIV preventative therapy may be an individual at risk of contracting the HIV virus.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

Except as otherwise noted, the methods and techniques of the present disclosure are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

In certain instances, the processes disclosed herein involve a step of forming a salt of a compound of the present disclosure.

Compounds as described herein can be purified by any of the means known in the art, including chromatographic means, such as high performance liquid chromatography (HPLC), preparative thin layer chromatography, flash column chromatography, supercritical fluid chromatography (SFC), and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2^{nd} ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, Protective Groups in Organic Synthesis, 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. One of skill in the art will recognize that the transformations shown in the schemes below may be performed in any order that is compatible with the functionality of the particular pendant groups. In some embodiments, each of the reactions depicted in the general schemes is run at a temperature from about −80° C. to the reflux temperature of the organic solvent used.

The compounds disclosed herein may display atropisomerism resulting from steric hindrance affecting the axial rotation rate around a single bond. The resultant conformational isomers may each be observed as distinct entities by characterization techniques such as NMR and HPLC. The compounds disclosed herein may exist as a mixture of atropisomers. However, the detection of atropisomers is dependent on factors such as temperature, solvent, conditions of purification, and timescale of spectroscopic technique. The interconversion rate at room temperature has a half-life of minutes to hours, hours to days, or days to years. The ratio of atropisomers at equilibrium may not be unity. Characterization data presented herein may not represent the equilibrium state depending on the conditions of isolation and characterization which may include but not limited to handling, solvents used, and temperature.

Compositions detailed herein may comprise a compound of the present disclosure in a racemic or non-racemic mixture of stereoisomers or may comprise a compound of the present disclosure as a substantially pure isomer. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

It is understood by one skilled in the art that this disclosure also includes any compound disclosed herein that may be enriched at any or all atoms above naturally occurring isotopic ratios with one or more isotopes such as, but not limited to, deuterium (2H or D).

Disclosed are also compounds in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Compounds of a given formula described herein encompasses the compound disclosed and all pharmaceutically acceptable salts, esters, stereoisomers, tautomers, prodrugs, solvates, and deuterated forms thereof, unless otherwise specified.

The compounds disclosed herein may contain chiral centers, which may be either of the (R) or (S) configuration, or which may comprise a mixture thereof. Accordingly, the present disclosure includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present disclosure.

The compounds of the present disclosure may be compounds according to Formula (I) with one or more chiral centers, which may be either of the (R) or (S) configuration, or which may comprise a mixture thereof.

The present disclosure includes both racemic mixtures of a compound disclosed herein and isolated isomers or any variation thereof. Where more than one chiral center is present in a compound of the present disclosure, some, none, or all of the chiral centers may be enantiomerically enriched. Thus, mixtures of a compound disclosed herein may be racemic with respect to one or more chiral centers and/or enantiomerically enriched with respect to one or more chiral centers.

### Compounds

The present disclosure provides in some embodiments a compound of Formula (I)

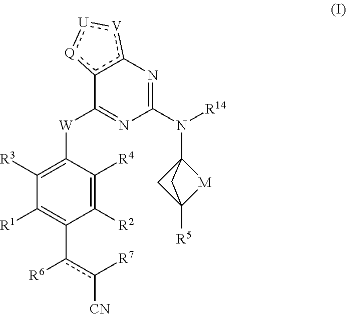

or a pharmaceutically acceptable salt thereof, wherein:
[symbol] is a single bond or a double bond;
R¹ is selected from H, CN, halogen, C₁₋₃alkyl, C₁₋₃haloalkyl, —OR^{1A}, —SR^{1A}, —NHR^{1A} and —N(R^{1A})₂;
R² is selected from H, CN, halogen, C₁₋₃alkyl, C₁₋₃haloalkyl, —OR^{2A}, —SR^{2A}, —NHR^{2A} and —N(R^{2A})₂;
R³ is selected from CN, halogen, C₁₋₆alkyl, C₁₋₆haloalkyl, —OR^{3A}, —CH₂OR^{3A}, —SR^{3A}, —NHR^{3A}, —N(R^{3A})₂, C₃₋₇cycloalkyl, and 3-7 membered heterocycloalkyl;
R⁴ is selected from CN, halogen, C₁₋₆alkyl, C₁₋₆haloalkyl, —OR^{4A}, —CH₂OR^{4A}, —SR^{4A}, —NHR^{4A}, —N(R^{4A})₂, C₃₋₇cycloalkyl, and 3-7 membered heterocycloalkyl;
or R¹ and R³ together form a 6-10 membered aryl;
R⁵ is selected from H, CN, halogen, C₁₋₆alkyl, C₁₋₆haloalkyl, C₂₋₆alkenyl, C₂₋₆alkynyl, —NHR^{5A}, —N(R^{5A})₂, —OR^{5A} C₃₋₇cycloalkyl, 3-10 membered heterocycloalkyl, —(C═O)NHR^{5A}, —(C═O)N(R^{5A})₂, and —(C═O)OR^{5A}; wherein the C₁₋₆alkyl, C₁₋₆haloalkyl, C₂₋₆alkenyl, C₂₋₆alkynyl, C₃₋₇cycloalkyl, and 3-10 membered heterocycloalkyl of R⁵ are each optionally substituted with 1 or 2 R⁸ groups;
R⁶ and R⁷ are independently selected from H and C₁₋₆alkyl; or R⁶ and R⁷ together form a C₃₋₇cycloalkyl or 3-7 membered heterocycloalkyl;
each R⁸ is independently selected from halogen, CN, C₁₋₃alkyl, C₁₋₃haloalkyl, C₁₋₃ alkoxyl, C₃₋₇cycloalkyl, and 3-7 membered heterocycloalkyl;
M is selected from —CH₂—, —CF₂—, —CH₂CH₂—, and —CH₂O—;
W is selected from —O—, —S—, —C(R^{W})₂—, —NR^{W}—, and —C(═O)—;
each R^{W} is independently selected from H, —OR^{W1}, C₁₋₆alkyl, C₃₋₇cycloalkyl, C₁₋₆ haloalkyl, and —(C═O)OR^{W1};
Q, U, and V are independently selected from N, O, S, CR^{Q}, C(R^{Q})₂, NR^{V}, N(R^{V})₂⁺, S═O, SO₂, C═CH₂, C═CHF, and C═O;
each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, —(C=O)OR^{Q1}, —(C═O)NHR^{Q2}, —(C═O)N(R^{Q2})₂, —NH(C═O)R^{Q1}, —NH(C═O)OR^{Q1}, —NH(C=O)NHR^{Q2}, —NH(C═O)N(R^{Q2})₂, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1, 2, 3, or 4 R⁹ groups;
each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, —(C═O)N(R^{V1})₂, —S(O)R^{V1}, and —S(O)₂R^{V1}; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1, 2, 3, or 4 R¹⁰ groups;
each R^{Q2} is independently selected from H, CN, C₁₋₆alkyl, C₁₋₆haloalkyl, C₁₋₆alkoxy, C₃₋₇ cycloalkyl, 4-7 membered heterocycloalkyl, 6-10 membered aryl, 5-10 membered heteroaryl, —(C=O)R^{X1}, —(C═O)OR^{X1}, —(C═O)NHR^{X1}, —(C═O)N(R^{X1})₂, and —(SO₂)R^{X1}; wherein each C₃₋₇ cycloalkyl, 4-7 membered heterocycloalkyl, 6-10 membered aryl, and 5-10 membered heteroaryl of R^{Q2} is optionally substituted with 1, 2, 3 or 4 R¹¹ groups;
or two R^{Q2} together form a 4-7 membered heterocycloalkyl;
or two R^{V} together form a 4-7 membered heterocycloalkyl;
or R^{Q} and R^{V} together form a 4-11 membered heterocycloalkyl or 5-10 membered heteroaryl, wherein the 4-11 membered heterocycloalkyl or 5-10 membered heteroaryl is optionally substituted with 1, 2, 3 or 4 R¹³ groups;
each R⁹ and R¹⁰ are independently selected from halogen, —OR^{X2}, CN, C₁₋₆alkyl, C₁₋₆haloalkyl, C₁₋₆alkoxy, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NR^{X2}, —(C═O)N(R^{X2})₂, and —(SO₂)R^{X2}; wherein each C₃₋₇cycloalkyl, 3-7 membered heterocycloalkyl, 6-10 membered aryl, and 5-10 membered heteroaryl of R⁹ and R¹⁰ are each optionally substituted with 1, 2, 3 or 4 R¹² groups;
each R¹¹, R¹² and R¹³ are independently selected from halogen, —OR^{X3}, CN, C₁₋₆alkyl, C₁₋₆haloalkyl, C₁₋₆alkoxyl, —NR^{X3}, —N(R^{X3})₂, C₃₋₇cycloalkyl, and 3-7 membered heterocycloalkyl; wherein each C₁₋₆alkyl of R¹¹, R¹² and R¹³ is optionally substituted with —OR^{X4};
R¹⁴ is selected from H and C₁₋₆alkyl; and
each R^{1A}, R^{2A}, R^{3A}, R^{4A}, R^{5A}, R^{Q1}, R^{V1}, R^{W1}, R^{X1}, R^{X2}, R^{X3}, and R^{X4} are independently selected from H, C₁₋₆alkyl, C₁₋₆haloalkyl, C₃₋₇cycloalkyl, and 6-10 membered aryl.

In some embodiments, the compound is a compound of Formula (Ia)

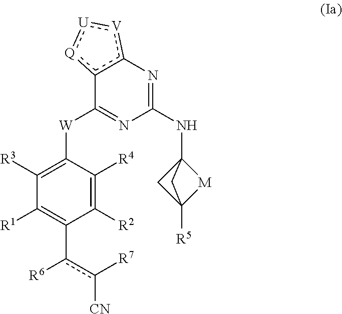

or a pharmaceutically acceptable salt thereof, wherein:
[symbol] is a single bond or a double bond;
R¹ is selected from H, CN, halogen, C₁₋₃alkyl, C₁₋₃haloalkyl, —OR^{1A}, —SR^{1A}, —NHR^{1A}, and —N(R^{1A})₂;
R² is selected from H, CN, halogen, C₁₋₃alkyl, C₁₋₃haloalkyl, —OR^{2A}, —SR^{2A}, —NHR^{2A}, and —N(R^{2A})₂;
R³ is selected from CN, halogen, C₁₋₆alkyl, C₁₋₆haloalkyl, —OR^{3A}, —CH₂OR^{3A}, —SR^{3A}, —NHR^{3A}, —N(R^{3A})₂, C₃₋₇cycloalkyl, and 3-7 membered heterocycloalkyl;
R⁴ is selected from CN, halogen, C₁₋₆alkyl, C₁₋₆haloalkyl, —OR^{4A}, —CH₂OR^{4A}, —SR^{4A}, —NHR^{4A}, —N(R^{4A})₂, C₃₋₇cycloalkyl, and 3-7 membered heterocycloalkyl;
R⁵ is selected from H, CN, halogen, C₁₋₆alkyl, C₁₋₆haloalkyl, C₂₋₆alkenyl, C₂₋₆alkynyl, —NHR^{5A}, —N(R^{5A})₂, —OR^{5A} C₃₋₇cycloalkyl, 3-10 membered heterocycloalkyl, —(C═O)NHR^{5A}, —(C═O)N(R^{5A})₂, and —(C═O)OR^{5A}; wherein the C₁₋₆alkyl, C₁₋₆haloalkyl, C₂₋₆alkenyl, C₂₋₆alkynyl, C₃₋₇cycloalkyl, and 3-10 membered heterocycloalkyl of R⁵ are each optionally substituted with 1 or 2 R⁸ groups;
R⁶ and R⁷ are independently selected from H and C₁₋₆alkyl; or R⁶ and R⁷ together form a C₃₋₇cycloalkyl or 3-7 membered heterocycloalkyl;
each R⁸ is independently selected from halogen, CN, C₁₋₃alkyl, C₁₋₃haloalkyl, C₁₋₃alkoxyl, C₃₋₇cycloalkyl, and 3-7 membered heterocycloalkyl;
M is selected from —CH₂—, —CF₂—, —CH₂CH₂—, and —CH₂O—;
W is selected from —O—, —S—, —C(R^{W})₂—, and —NR^{W}—;
each R^{W} is independently selected from H, C₁₋₆alkyl, C₃₋₇cycloalkyl, and C₁₋₆haloalkyl;
Q, U, and V are independently selected from N, O, S, CR^{Q}, C(R^{Q})₂, NR^{V}, N(R^{V})₂⁺, SO₂, C═CH₂, and C═O;
each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, —(C═O)OR^{Q1}, —(C═O)NHR^{Q2}, —(C═O)N(R^{Q2})₂, —NH(C═O)R^{Q1}, —NH(C═O)OR^{Q1}, —NH(C═O)NHR^{Q2}, —NH(C═O)N(R^{Q2})₂, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1, 2, 3, or 4 R⁹ groups;
each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, —(C═O)N(R^{V1})₂, —S(O)R^{V1}, and —S(O)₂R^{V1}; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1, 2, 3, or 4 R¹⁰ groups;
each R^{Q2} is independently selected from H, C₁₋₆alkyl, C₁₋₆haloalkyl, C₁₋₆alkoxy, C₃₋₇cycloalkyl, 4-7 membered heterocycloalkyl, 6-10 membered aryl, 5-10 membered heteroaryl, —(C═O)R^{X1}, —(C═O)OR^{X1}, —(C═O)NHR^{X1}, —(C═O)N(R^{X1})₂, and —(SO₂)R^{X1}; wherein the C₃₋₇cycloalkyl, 4-7 membered heterocycloalkyl, 6-10 membered aryl, and 5-10 membered heteroaryl of R^{Q2} is optionally substituted with 1, 2, 3 or 4 R¹¹ groups;
or two R^{Q2} together form a 4-7 membered heterocycloalkyl;
or R^{Q} and R^{V} together form a 4-7 membered heterocycloalkyl or 5-10 membered heteroaryl, wherein the 4-7 membered heterocycloalkyl or 5-10 membered heteroaryl is optionally substituted with 1, 2, 3 or 4 R¹³ groups;
each R⁹ and R¹⁰ are independently selected from halogen, —OR^{X2}, CN, C₁₋₆alkyl, C₁₋₆haloalkyl, C₁₋₆alkoxy, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NHR^{X2}, —(C═O)N(R^{X2})₂, and —(SO₂)R^{X2}; wherein the C₃₋₇cycloalkyl, 3-7 membered heterocycloalkyl, 6-10 membered aryl, and 5-10 membered heteroaryl of R⁹ and R¹⁰ are each optionally substituted with 1, 2, 3 or 4 R¹² groups;
each R¹¹, R¹² and R¹³ are independently selected from halogen, —OR^{X3}, CN, C₁₋₆alkyl, C₁₋₆haloalkyl, C₁₋₆alkoxyl, —NR^{X3}, —N(R^{X3})₂, C₃₋₇cycloalkyl, and 3-7 membered heterocycloalkyl; and
each R^{1A}, R^{2A}, R^{3A}, R^{4A}, R^{5A}, R^{Q1}, R^{V1}, R^{X1}, R^{X2} and R^{X3} are independently selected from H, C₁₋₆alkyl, C₁₋₆haloalkyl, and C₃₋₇cycloalkyl.

In some embodiments, W is selected from —O—, —NR^{W}—, —C(R^{W})₂—, and —C(═O)—. In some embodiments, W is selected from —O— and —NR^{W}—. In some embodiments, W is —NR^{W}—. In some embodiments, R^{W} is selected from H and —OR^{W1}.

In some embodiments, W is NH. In some embodiments, W is —CH(OH)—. In some embodiments, W is O. In some embodiments, W is C═O.

In some embodiments, R¹ is selected from H, CN, halogen, C₁₋₃alkyl, C₁₋₃haloalkyl, —OR^{1A}, and —SR^{1A}. In some embodiments, R¹ is selected from H, halogen, C₁₋₃alkyl, and C₁₋₃ haloalkyl. In some embodiments, R¹ is selected from H, C₁₋₃alkyl, and C₁₋₃haloalkyl. In some embodiments, R¹ is H. In some embodiments, R¹ is C₁₋₃alkyl. In some embodiments, R¹ is C₁₋₃haloalkyl. In some embodiments, R¹ is selected from H, methyl, and trifluoromethyl.

In some embodiments, R² is selected from H, CN, halogen, C₁₋₃alkyl, C₁₋₃haloalkyl, —OR^{1A}, and —SR^{1A}. In some embodiments, R² is selected from H, halogen, C₁₋₃alkyl, and C₁₋₃ haloalkyl. In some embodiments, R² is selected from H, C₁₋₃alkyl, and C₁₋₃haloalkyl. In some embodiments, R² is H. In some embodiments, R² is C₁₋₃alkyl. In some embodiments, R² is C₁₋₃haloalkyl. In some embodiments, R² is selected from H, methyl, and trifluoromethyl. In some embodiments, both R¹ and R² are H.

In some embodiments, R³ is selected from CN, halogen, C₁₋₆alkyl, C₁₋₆haloalkyl, —OR^{3A}, —CH₂OR^{3A}, —SR^{3A}, —NHR^{3A} and —N(R^{3A})₂. In some embodiments, R³ is selected from CN, halogen, C₁₋₆alkyl, C₁₋₆haloalkyl, —OR^{3A}, and —SR^{3A}. In some embodiments, R³ is selected from halogen, C₁₋₆alkyl, C₁₋₆haloalkyl, —OR^{3A}, and —CH₂OR^{3A}. In some embodiments, R³ is halogen. In some embodiments, R³ is C₁₋₆alkyl. In some embodiments, R³ is C₁₋₆haloalkyl. In some embodiments, R³ is —OR^{3A}. In some embodiments, R³ is —CH₂OR^{3A}. In some embodiments, R³ is selected from methyl, ethyl, fluoro, chloro, difluoromethyl, methoxyl, and ethoxyl. In some embodiments, R³ is methyl.

In some embodiments, R⁴ is selected from CN, halogen, C₁₋₆alkyl, C₁₋₆haloalkyl, —OR^{4A}, —CH₂OR^{4A}, —SR^{4A}, —NHR^{4A} and —N(R^{4A})₂. In some embodiments, R⁴ is selected from CN, halogen, C₁₋₆alkyl, C₁₋₆haloalkyl, —OR^{4A}, and —SR^{4A}. In some embodiments, R⁴ is selected from halogen, C₁₋₆alkyl, C₁₋₆haloalkyl, —OR^{4A}. In some embodiments, R⁴ is halogen. In some embodiments, R⁴ is C₁₋₆alkyl. In some embodiments, R⁴ is C₁₋₆haloalkyl. In some embodiments, R⁴ is —OR^{4A}. In some embodiments, R⁴ is selected from methyl, ethyl, fluoro, chloro, difluoromethyl, methoxyl, and ethoxyl. In some embodiments, R⁴ is methyl. In some embodiments, both R³ and R⁴ are selected from methyl, ethyl, fluoro, chloro, difluoromethyl, methoxyl, and ethoxyl. In some embodiments, both R³ and R⁴ are methyl.

In some embodiments, R⁵ is selected from H, CN, halogen, C₁₋₆alkyl, C₁₋₆haloalkyl, C₂₋₆alkenyl, C₂₋₆alkynyl, C₃₋₇cycloalkyl, 3-10 membered heterocycloalkyl; wherein the C₁₋₆alkyl, C₁₋₆haloalkyl, C₂₋₆alkenyl, C₂₋₆alkynyl, C₃₋₇cycloalkyl, and 3-10 membered heterocycloalkyl of R⁵ are each optionally substituted with 1 or 2 R⁸ groups. In some embodiments, R⁵ is selected from H, CN, C₁₋₆alkyl, C₁₋₆haloalkyl, C₃₋₇cycloalkyl, 3-10 membered heterocycloalkyl; wherein the C₁₋₆alkyl, C₁₋₆haloalkyl, C₂₋₆alkenyl, C₂₋₆alkynyl, C₃₋₇cycloalkyl, and 3-10 membered heterocycloalkyl of R⁵ are each optionally substituted with 1 R⁸ group. In some embodiments, R⁵ is selected from H, CN, C₁₋₆haloalkyl, 3-10 membered heterocycloalkyl. In some embodiments, R⁵ is H. In some embodiments, R⁵ is CN. In some embodiments, R⁵ is C₁₋₆haloalkyl. In some embodiments, R⁵ is 3-10 membered heterocycloalkyl. In some embodiments, R⁵ is selected from H, CN, difluoroethyl, and oxetanyl. In some embodiments, R⁵ is CN.

In some embodiments, R⁶ and R⁷ are independently selected from H and C₁₋₆alkyl; or R⁶ and R⁷ together form a C₃₋₇cycloalkyl. In some embodiments, R⁷ is C₁₋₆alkyl. In some embodiments, R⁶ is H and R⁷ is methyl. In some embodiments, R⁶ and R⁷ are H. In some embodiments, R⁶ and R⁷ together form a C₃₋₇cycloalkyl. In some embodiments, R⁶ and R⁷ together form a cyclopropyl.

In some embodiments, both R¹ and R² are H, both R³ and R⁴ are methyl, both R⁵ is CN, and both R⁶ and R⁷ are H.

In some embodiments, both R¹ and R² are H, both R³ and R⁴ are methyl, R⁵ is CN, both R⁶ and R⁷ are H, and W is O.

In some embodiments, M is selected from —CH₂— and —CF₂—. In some embodiments, M is —CH₂—. In some embodiments, M is —CF₂—.

In some embodiments, both R¹ and R² are H, both R³ and R⁴ are methyl, R⁵ is CN, both R⁶ and R⁷ are H, W is O, and M is —CH₂—.

In some embodiments, Q, U, and V are independently selected from N, O, S, CR^{Q}, C(R^{Q})₂, NR^{V}, N(R^{V})₂⁺, S═O, C═CH₂, C═CHF, and C═O. In some embodiments, Q, U, and V are independently selected from N, O, S, CR^{Q}, C(R^{Q})₂, NR^{V}, N(R^{V})₂⁺, C═CH₂, and C═O. In some embodiments, at least one of Q, U, and V is N. In some embodiments, at least one of Q, U, and V is O. In some embodiments, at least one of Q, U, and V is S. In some embodiments, at least one of Q, U, and V is CR^{Q}. In some embodiments, at least one of Q, U, and V is C(R^{Q})₂. In some embodiments, at least one of Q, U, and V is NR^{V}. In some embodiments, at least one of Q, U, and V is N(R^{V})₂⁺. In some embodiments, at least one of Q, U, and V is C═O. In some embodiments, at least one of Q, U, and V is C═CH₂. In some embodiments, at least one of Q, U, and V is C═CHF. In some embodiments, at least one of Q, U, and V is S═O. In some embodiments, V is C═O, and U is NR^{V}.

In some embodiments, each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, —NH(C═O)R^{Q1}, —NH(C═O)OR^{Q1}, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1 or 2 R⁹ groups.

In some embodiments, each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1 or 2 R⁹ groups. In some embodiments, each R^{Q} is independently selected from H, halogen, —OR^{Q2}, CN, C₁₋₆alkyl, C₁₋₆haloalkyl, C₂₋₆alkenyl, C₃₋₇cycloalkyl, 3-7 membered heterocycloalkyl, —NHR^{Q2}, and 5-10 membered heteroaryl; wherein each C₁₋₆alkyl, C₁₋₆haloalkyl, C₂₋₆alkenyl, C₃₋₇cycloalkyl, 3-7 membered heterocycloalkyl, and 5-10 membered heteroaryl of R^{Q} are each optionally substituted with 1 or 2 R⁹ groups. In some embodiments, each R^{Q} is H. In some embodiments, each R^{Q} is halogen. In some embodiments, each R^{Q} is —OR^{Q2}. In some embodiments, each R^{Q} is CN. In some embodiments, each R^{Q} is C₁₋₆alkyl optionally substituted with 1 or 2 R⁹ groups. In some embodiments, each R^{Q} is C₁₋₆haloalkyl optionally substituted with 1 or 2 R⁹ groups. In some embodiments, each R^{Q} is C₂₋₆alkenyl optionally substituted with 1 or 2 R⁹ groups. In some embodiments, each R^{Q} is C₃₋₇cycloalkyl optionally substituted with 1 or 2 R⁹ groups. In some embodiments, each R^{Q} is 3-7 membered heterocycloalkyl optionally substituted with 1 or 2 R⁹ groups. In some embodiments, each R^{Q} is —NHR^{Q2}. In some embodiments, each R^{Q} is 5-10 membered heteroaryl optionally substituted with 1 or 2 R⁹ groups. In some embodiments, R^{Q} is selected from H, CN, OH, F, Cl, Br, I, NH₂, NHR^{Q2}, methyl, ethyl, ethylenyl, isopropyl, cyclopropyl, cyclobutyl, cyclopentyl, cyclohexyl, fluoromethyl, difluoromethyl, trifluoromethyl, fluoroethyl, difluoroethyl, trifluoroethyl, tetrahydropyranyl, oxazolyl, pyrazolyl, and pyrimidinyl.

In some embodiments, each R^{Q2} is independently selected from H, C₁₋₆alkyl, C₁₋₆haloalkyl, C₁₋₆alkoxy, C₃₋₇cycloalkyl, 4-7 membered heterocycloalkyl, 6-10 membered aryl, 5-10 membered heteroaryl, —(C═O)R^{X1}, —(C═O)OR^{X1}, —(C═O)NHR^{X1}, —(C═O)N(R^{X1})₂, and —(SO₂)R^{X1}; wherein each C₃₋₇cycloalkyl, 4-7 membered heterocycloalkyl, 6-10 membered aryl, and 5-10 membered heteroaryl of R^{Q2} is optionally substituted with 1 or 2 R¹¹ groups; or two R^{Q2} together form a 4-7 membered heterocycloalkyl. In some embodiments, each R^{Q2} is independently selected from H, C₁₋₆alkyl, C₁₋₆haloalkyl, C₁₋₆alkoxy, C₃₋₇cycloalkyl, 4-7 membered heterocycloalkyl, 6-10 membered aryl, 5-10 membered heteroaryl; wherein each C₃₋₇cycloalkyl, 4-7 membered heterocycloalkyl, 6-10 membered aryl, and 5-10 membered heteroaryl of R^{Q2} is optionally substituted with 1 or 2 R¹¹ groups; or two R^{Q2} together form a 4-7 membered heterocycloalkyl. In some embodiments, each R^{Q2} is independently selected from H, C₁₋₆alkyl, C₁₋₆haloalkyl, C₁₋₆alkoxy, 6-10 membered aryl, 5-10 membered heteroaryl; wherein each 6-10 membered aryl and 5-10 membered heteroaryl of R^{Q2} is optionally substituted with 1 or 2 R¹¹ groups. In some embodiments, each R^{Q2} is independently selected from H, C₁₋₆alkyl, C₁₋₆ alkoxy, 6-10 membered aryl, 5-10 membered heteroaryl; wherein each 6-10 membered aryl and 5-10 membered heteroaryl of R^{Q2} is optionally substituted with 1 or 2 R¹¹ groups. In some embodiments, each R^{Q2} is H. In some embodiments, each R^{Q2} is C₁₋₆alkyl. In some embodiments, each R^{Q2} is C₁₋₆alkoxy. In some embodiments, each R^{Q2} is 6-10 membered aryl optionally substituted with 1 or 2 R¹¹ groups. In some embodiments, each R^{Q2} is 5-10 membered heteroaryl optionally substituted with 1 or 2 R¹¹ groups. In some embodiments, each R^{Q2} is 5-10 membered heteroaryl optionally substituted with 1 or 2 R¹¹ groups. In some embodiments, R^{Q2} is selected from H, CN, methyl, and methoxyl.

In some embodiments, each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, and —(C═O)N(R^{V1})₂; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1, 2, or 3 R¹⁰ groups. In some embodiments, each R^{V} is independently selected from H, C₁₋₆alkyl, C₁₋₆haloalkyl, C₂₋₆alkenyl, C₂₋₆alkynyl, C₃₋₇ cycloalkyl, 4-7 membered heterocycloalkyl, 6-10 membered aryl, 5-10 membered heteroaryl, —(C═O)R^{V1}, —(C═O)OR^{V1}, —(C═O)NR^{V1}, and —(C═O)N(R^{V1})₂; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1 or 2 R¹⁰ groups. In some embodiments, each R^{V} is independently selected from H, C₁₋₆alkyl, C₁₋₆ haloalkyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, and —(C═O)N(R^{V1})₂; wherein each C₁₋₆alkyl, C₁₋₆haloalkyl, C₃₋₇cycloalkyl, 4-7 membered heterocycloalkyl, 6-10 membered aryl, and 5-10 membered heteroaryl of R^{V} are each optionally substituted with 1 or 2 R¹⁰ groups. In some embodiments, each R^{V} is independently selected from H, C₁₋₆alkyl, C₁₋₆haloalkyl, C₃₋₇ cycloalkyl, 4-7 membered heterocycloalkyl, 6-10 membered aryl, and —(C═O)OR^{V1}; wherein each C₁₋₆alkyl, C₁₋₆haloalkyl, C₃₋₇cycloalkyl, 4-7 membered heterocycloalkyl, and 6-10 membered aryl of R^{V} are each optionally substituted with 1 or 2 R¹⁰ groups.

In some embodiments, each R^{V} is independently selected from H, C₁₋₆alkyl, C₁₋₆haloalkyl, C₂₋₆alkenyl, C₃₋₇cycloalkyl, 4-7 membered heterocycloalkyl, 6-10 membered aryl, 5-10 membered heteroaryl, and —(C═O)OR^{V1}; wherein each C₁₋₆alkyl, C₁₋₆haloalkyl, C₃₋₇cycloalkyl, 4-7 membered heterocycloalkyl, and 6-10 membered aryl of R^{V} are each optionally substituted with 1, 2, or 3 R¹⁰ groups. In some embodiments, each R^{V} is H. In some embodiments, each R^{V} is C₁₋₆alkyl optionally substituted with 1 or 2 R¹⁰ groups. In some embodiments, each R^{V} is C₁₋₆haloalkyl optionally substituted with 1 or 2 R¹⁰ groups. In some embodiments, each R^{V} is C₂₋₆alkenyl. In some embodiments, each R^{V} is C₃₋₇cycloalkyl optionally substituted with 1 or 2 R¹⁰ groups. In some embodiments, each R^{V} is 4-7 membered heterocycloalkyl optionally substituted with 1 or 2 R¹⁰ groups. In some embodiments, each R^{V} is 6-10 membered aryl optionally substituted with 1 or 2 R¹⁰ groups. In some embodiments, each R^{V} is —(C═O)OR^{V1}. In some embodiments, each R^{V} is independently selected from H, methyl, ethyl, isopropyl, cyclopropyl, cyclobutyl, fluoromethyl, difluoromethyl, trifluoromethyl, fluoroethyl, difluoroethyl, trifluoroethyl, phenyl, and —(C═O)OR^{V1}.

In some embodiments, R^{V1} is selected from H, C₁₋₆alkyl, C₁₋₆haloalkyl, and C₃₋₇ cycloalkyl. In some embodiments, R^{V1} is C₁₋₆alkyl. In some embodiments, R^{V1} is methyl or tert-butyl.

In some embodiments, two R^{V} together form a 4-7 membered heterocycloalkyl. In some embodiments, R^{Q} and R^{V} together form a 4-11 membered heterocycloalkyl or 5-10 membered heteroaryl, wherein the 4-11 membered heterocycloalkyl or 5-10 membered heteroaryl is optionally substituted with 1 or 2 R¹³ groups.

In some embodiments, each R⁹ and R¹⁰ are independently selected from halogen, —OR^{X2}, CN, C₁₋₆alkyl, C₁₋₆haloalkyl, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NR^{X2}, and —(C═O)N(R^{X2})₂; wherein the C₃₋₇cycloalkyl, 3-7 membered heterocycloalkyl, 6-10 membered aryl, and 5-10 membered heteroaryl of R⁹ and R¹⁰ are each optionally substituted with 1 or 2 R¹² groups. In some embodiments, each R⁹ and R¹⁰ are independently selected from halogen, —OR^{X2}, CN, C₁₋₆alkyl, C₁₋₆haloalkyl, C₃₋₇cycloalkyl, 6-10 membered aryl, 5-10 membered heteroaryl, —(C═O)NHR^{X2}, and —(C═O)N(R^{X2})₂; wherein the C₃₋₇cycloalkyl, 6-10 membered aryl, and 5-10 membered heteroaryl of R⁹ and R¹⁰ are each optionally substituted with 1 or 2 R¹² groups. In some embodiments, each R⁹ and R¹⁰ are independently selected from halogen, —OR^{X2}, CN, C₁₋₆alkyl, C₁₋₆haloalkyl, C₃₋₇cycloalkyl, 3-7 membered heterocycloalkyl, 6-10 membered aryl, 5-10 membered heteroaryl, and —(C═O)NR^{X2}; wherein the C₃₋₇cycloalkyl, 6-10 membered aryl, and 5-10 membered heteroaryl of R⁹ and R¹⁰ are each optionally substituted with 1 or 2 R¹² groups. In some embodiments, each R⁹ and R¹⁰ are independently selected from halogen, —OR^{X2}, CN, C₁₋₆alkyl, C₃₋₇cycloalkyl, 6-10 membered aryl, 5-10 membered heteroaryl, and —(C═O)NR^{X2}; wherein the C₃₋₇cycloalkyl, 6-10 membered aryl, and 5-10 membered heteroaryl of R⁹ and R¹⁰ are each optionally substituted with 1 or 2 R¹² groups. In some embodiments, each R⁹ and R¹⁰ are independently selected from fluoro, hydroxyl, methoxyl, CN, methyl, trifluoromethyl, cyclopropyl, morpholinyl, phenyl, pyrazole, and —(C═O)NR^{X2}. In some embodiments, each R⁹ and R¹⁰ are independently selected from fluoro, hydroxyl, methoxyl, CN, methyl, cyclopropyl, phenyl, pyrazole, and —(C═O)NR^{X2}.

In some embodiments, each R¹¹, R¹², and R¹³ are independently selected from halogen, —OR^{X3}, CN, C₁₋₆alkyl, C₁₋₆haloalkyl, C₁₋₆alkoxyl, C₃₋₇cycloalkyl, and 3-7 membered heterocycloalkyl; wherein each C₁₋₆alkyl of R¹¹, R¹² and R¹³ is optionally substituted with —OR^{X4}. In some embodiments, each R¹¹ and R¹² are independently selected from halogen, —OR^{X3}, CN, C₁₋₆alkyl, C₁₋₆haloalkyl, C₁₋₆alkoxyl, C₃₋₇cycloalkyl, and 3-7 membered heterocycloalkyl.

In some embodiments, each R¹¹, R¹², and R¹³ are independently selected from halogen, —OR^{X3}, CN, C₁₋₆alkyl, C₁₋₆haloalkyl, and C₁₋₆alkoxyl wherein each C₁₋₆alkyl of R¹¹, R¹² and R¹³ is optionally substituted with —OR^{X4}. In some embodiments, each R¹¹ and R¹² are independently selected from halogen, —OR^{X3}, CN, C₁₋₆alkyl, C₁₋₆haloalkyl, and C₁₋₆alkoxyl. In some embodiments, each R¹, R¹² and R¹³ are independently selected from halogen.

In some embodiments, each R¹¹, R¹², and R¹³ are independently selected from —OR^{X3} and C₁₋₆alkyl wherein each C₁₋₆alkyl of R¹¹, R¹² and R¹³ is optionally substituted with —OR^{X4}. In some embodiments, each R¹¹ and R¹² are independently selected from —OR^{X3} and C₁₋₆alkyl. In some embodiments, each R¹, R¹² and R¹³ are independently selected from methoxyl and methyl. In some embodiments, each R¹¹ and R¹² are independently selected from methoxyl and methyl.

In some embodiments, R¹⁴ is H. In some embodiments, R¹⁴ is C₁₋₆alkyl.

In some embodiments, each R^{1A}, R^{2A}, R^{3A}, R^{4A}, R^{5A}, R^{Q1}, R^{V1}, R^{W1}, R^{X1}, R^{X2}, R^{X3}, and R^{X4} are independently selected from H, C₁₋₆alkyl, C₁₋₆haloalkyl, and C₃₋₇cycloalkyl. In some embodiments, each R^{1A}, R^{2A}, R^{3A}, R^{4A}, R^{5A}, R^{Q1}, R^{X1}, R^{X2} and R^{X3} are independently selected from H, C₁₋₆alkyl, C₁₋₆haloalkyl, and C₃₋₇cycloalkyl. In some embodiments, each R^{1A}, R^{2A}, R^{3A}, R^{4A}, R^{5A}, R^{Q1}, R^{V1}, R^{W1}, R^{X1}, R^{X2}, R^{X3}, and R^{X4} are independently selected from H, C₁₋₆alkyl, and C₃₋₇cycloalkyl. In some embodiments, each R^{1A}, R^{2A}, R^{3A}, R^{4A}, R^{5A}, R^{Q1}, R^{X1}, R^{X2} and R^{X3} are independently selected from H, C₁₋₆alkyl, and C₃₋₇cycloalkyl. In some embodiments, each R^{1A}, R^{2A}, R^{3A}, R^{4A}, R^{5A}, R^{Q1}, R^{V1}, R^{W1}, R^{X1}, R^{X2}, R^{X3}, and R^{X4} are independently selected from H and C₁₋₆alkyl. In some embodiments, each R^{1A}, R^{2A}, R^{3A}, R^{4A}, R^{5A}, R^{Q1}, R^{X1}, R^{X2} and R^{X3} are independently selected from H and C₁₋₆alkyl. In some embodiments, R^{X2} is methyl. In some embodiments, R^{X3} is methyl.

Some embodiments provide a compound of Formula (I), or a pharmaceutically acceptable salt thereof, wherein:
[symbol] is a single bond or a double bond;
R¹ is selected from H, C₁₋₃alkyl, and C₁₋₃haloalkyl;
R² is selected from H, C₁₋₃alkyl, and C₁₋₃haloalkyl;
R³ is selected from halogen, C₁₋₆alkyl, C₁₋₆haloalkyl, —OR^{3A}, —SR^{3A}, —NHR^{3A} and —N(R^{3A})₂;
R⁴ is selected from halogen, C₁₋₆alkyl, C₁₋₆haloalkyl, —OR^{4A}, —SR^{4A}, —NHR^{4A} and —N(R^{4A})₂;
R⁵ is selected from H, CN, C₁₋₆alkyl, C₁₋₆haloalkyl, C₃₋₇cycloalkyl, and 3-10 membered heterocycloalkyl;
R⁶ and R⁷ are independently selected from H and C₁₋₆alkyl; or R⁶ and R⁷ together form a C₃₋₇cycloalkyl or 3-7 membered heterocycloalkyl;
M is selected from —CH₂—, —CF₂—, —CH₂CH₂—, and —CH₂O—;
W is selected from —O—, —S—, —C(R^{W})₂—, —NR^{W}—, and —C(═O)—;
each R^{W} is independently selected from H, —OR^{W1}, and C₁₋₆alkyl;
Q, U, and V are independently selected from N, O, S, CR^{Q}, C(R^{Q})₂, NR^{V}, N(R^{V})₂⁺, S═O, SO₂, C═CH₂, C═CHF, and C═O;
each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1 or 2 R⁹ groups;
each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, —(C═O)N(R^{V1})₂, —S(O)R^{V1}, and —S(O)₂R^{V1}; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1, 2, or 3 R¹⁰ groups;
each R^{Q2} is independently selected from H, C₁₋₆alkyl, C₁₋₆haloalkyl, C₁₋₆alkoxy, C₃₋₇ cycloalkyl, 4-7 membered heterocycloalkyl, 6-10 membered aryl, 5-10 membered heteroaryl; wherein the C₃₋₇cycloalkyl, 4-7 membered heterocycloalkyl, 6-10 membered aryl, and 5-10 membered heteroaryl of R^{Q2} is optionally substituted with 1 or 2 R¹¹ groups;
or two R^{V} together form a 4-7 membered heterocycloalkyl;
or R^{Q} and R^{V} together form a 4-11 membered heterocycloalkyl or 5-10 membered heteroaryl, wherein the 4-11 membered heterocycloalkyl or 5-10 membered heteroaryl is optionally substituted with 1 or 2 R¹³ groups
each R⁹ and R¹⁰ are independently selected from halogen, —OR^{X2}, CN, C₁₋₆alkyl, C₁₋₆haloalkyl, C₁₋₆alkoxy, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NR^{X2}, —(C═O)N(R^{X2})₂, and —(SO₂)R^{X2}; wherein the C₃₋₇cycloalkyl, 3-7 membered heterocycloalkyl, 6-10 membered aryl, and 5-10 membered heteroaryl of R⁹ and R¹⁰ are each optionally substituted with 1 or 2 R¹² groups;
each R¹¹, R¹², and R¹³ are independently selected from halogen, —OR^{X3}, CN, C₁₋₆alkyl, C₁₋₆haloalkyl, and C₁₋₆alkoxyl; wherein each C₁₋₆alkyl of R¹¹, R¹² and R¹³ is optionally substituted with —OR^{X4};
R¹⁴ H; and
each R^{3A}, R^{4A}, R^{V1}, R^{W1}, R^{X2}, R^{X3}, and R^{X4} are independently selected from H, C₁₋₆alkyl, C₁₋₆haloalkyl, and C₃₋₇cycloalkyl.

Some embodiments provide a compound of Formula (Ia), or a pharmaceutically acceptable salt thereof, wherein:
[symbol] is a single bond or a double bond;
R¹ is selected from H, C₁₋₃alkyl, and C₁₋₃haloalkyl;
R² is selected from H, C₁₋₃alkyl, and C₁₋₃haloalkyl;
R³ is selected from halogen, C₁₋₆alkyl, C₁₋₆haloalkyl, —OR^{3A}, —CH₂OR^{3A}, —SR^{3A}, —NHR^{3A}, and —N(R^{3A})₂;
R⁴ is selected from halogen, C₁₋₆alkyl, C₁₋₆haloalkyl, —OR^{4A}, —CH₂OR^{4A}, —SR^{3A}, —NHR^{3A}, and —N(R^{3A})₂;
R⁵ is selected from H, CN, C₁₋₆alkyl, C₁₋₆haloalkyl, C₃₋₇cycloalkyl, and 3-10 membered heterocycloalkyl;
R⁶ and R⁷ are independently selected from H and C₁₋₆alkyl; or R⁶ and R⁷ together form a C₃₋₇cycloalkyl or 3-7 membered heterocycloalkyl;
M is selected from —CH₂—, —CF₂—, —CH₂CH₂—, and —CH₂O—;
W is selected from —O—, —S—, —C(R^{W})₂—, and —NR^{W}—;
each R^{W} is independently selected from H and C₁₋₆alkyl;
Q, U, and V are independently selected from N, O, S, CR^{Q}, C(R^{Q})₂, NR^{V}, N(R^{V})₂⁺, SO₂, C═CH₂, and C═O;
each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1 or 2 R⁹ groups;
each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, —(C═O)N(R^{V1})₂, —S(O)R^{V1}, and —S(O)₂R^{V1}; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1 or 2 R¹⁰ groups;
each R^{Q2} is independently selected from H, C₁₋₆alkyl, C₁₋₆haloalkyl, C₁₋₆alkoxy, C₃₋₇ cycloalkyl, 4-7 membered heterocycloalkyl, 6-10 membered aryl, 5-10 membered heteroaryl; wherein the C₃₋₇cycloalkyl, 4-7 membered heterocycloalkyl, 6-10 membered aryl, and 5-10 membered heteroaryl of R^{Q2} is optionally substituted with 1 or 2 R¹¹ groups;
each R⁹ and R¹⁰ are independently selected from halogen, —OR^{X2}, CN, C₁₋₆alkyl, C₁₋₆haloalkyl, C₁₋₆alkoxy, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NR^{X2}, —(C═O)N(R^{X2})₂, and —(SO₂)R^{X2}; wherein the C₃₋₇cycloalkyl, 3-7 membered heterocycloalkyl, 6-10 membered aryl, and 5-10 membered heteroaryl of R⁹ and R¹⁰ are each optionally substituted with 1 or 2 R¹² groups;
each R¹¹ and R¹² are independently selected from halogen, —OR^{X3}, CN, C₁₋₆alkyl, C₁₋₆haloalkyl, and C₁₋₆alkoxyl; and each R^{3A}, R^{4A}, R^{Q1}, R^{V1}, R^{X1}, R^{X2} and R^{X3} are independently selected from H, C₁₋₆alkyl, C₁₋₆haloalkyl, and C₃₋₇cycloalkyl.

Some embodiments provide a compound of Formula (I), or a pharmaceutically acceptable salt thereof, wherein:
[symbol] is a single bond or a double bond;
R¹ is selected from H and C₁₋₃haloalkyl;
R² is selected from H and C₁₋₃haloalkyl;
R³ is selected from halogen, C₁₋₆alkyl, C₁₋₆haloalkyl, and —OR^{3A};
R⁴ is selected from halogen, C₁₋₆alkyl, C₁₋₆haloalkyl, and —OR^{4A};
R⁵ is selected from H, CN, C₁₋₆haloalkyl, 3-10 membered heterocycloalkyl;
R⁶ and R⁷ are independently selected from H and C₁₋₆alkyl; or R⁶ and R⁷ together form a C₃₋₇cycloalkyl;
M is selected from —CH₂— and —CF₂—;
W is selected from —O—, —CH(OH)—, —NH—, and —C(═O)—;
Q, U, and V are independently selected from N, O, S, CR^{Q}, C(R^{Q})₂, NR^{V}, N(R^{V})₂⁺, C═CH₂, and C═O;
each R^{Q} is independently selected from H, halogen, —OR^{Q2}, CN, C₁₋₆alkyl, C₁₋₆haloalkyl, C₂₋₆alkenyl, C₃₋₇cycloalkyl, 3-7 membered heterocycloalkyl, —NHR^{Q2}, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, and 5-10 membered heteroaryl of R^{Q} are each optionally substituted with 1 or 2 R⁹ groups;
each R^{V} is independently selected from H, C₁₋₆alkyl, C₁₋₆haloalkyl, C₂₋₆alkenyl, C₃₋₇ cycloalkyl, 4-7 membered heterocycloalkyl, 6-10 membered aryl, 5-10 membered heteroaryl, and —(C═O)OR^{V1}; wherein each C₁₋₆alkyl, C₁₋₆haloalkyl, C₃₋₇cycloalkyl, and 4-7 membered heterocycloalkyl of R^{V} are each optionally substituted with 1, 2, or 3 R¹⁰ groups;
each R^{Q2} is independently selected from H, C₁₋₆alkyl, C₁₋₆haloalkyl, 6-10 membered aryl, and 5-10 membered heteroaryl; wherein the 6-10 membered aryl, and 5-10 membered heteroaryl of R^{Q2} is optionally substituted with 1 or 2 R¹¹ groups;
or two R^{V} together form a 4-7 membered heterocycloalkyl;
or R^{Q} and R^{V} together form a 4-11 membered heterocycloalkyl or 5-10 membered heteroaryl, wherein the 4-11 membered heterocycloalkyl or 5-10 membered heteroaryl is optionally substituted with 1, 2, 3 or 4 R¹³ groups;
each R⁹ and R¹⁰ are independently selected from halogen, —OR^{X2}, CN, C₁₋₆alkyl, C₁₋₆haloalkyl, C₃₋₇cycloalkyl, 3-7 membered heterocycloalkyl, 6-10 membered aryl, 5-10 membered heteroaryl, and —(C═O)NR^{X2}; wherein the C₃₋₇cycloalkyl, 6-10 membered aryl, and 5-10 membered heteroaryl of R⁹ and R¹⁰ are each optionally substituted with 1 or 2 R¹² groups;
each R¹¹, R¹², and R¹³ are independently selected from halogen, —OR^{X3}, CN, and C₁₋₆alkyl; wherein each C₁₋₆alkyl of R¹¹, R¹² and R¹³ is optionally substituted with —OR^{X4};
R¹⁴ is H; and
each R^{3A}, R^{4A}, R^{V1}, R^{X2} R^{X3}, and R^{X4} are independently selected from H and C₁₋₆alkyl.

Some embodiments provide a compound of Formula (Ia), or a pharmaceutically acceptable salt thereof, wherein:
[symbol] is a single bond or a double bond;
R¹ is selected from H and C₁₋₃haloalkyl;
R² is selected from H and C₁₋₃haloalkyl;
R³ is selected from halogen, C₁₋₆alkyl, C₁₋₆haloalkyl, and —OR^{3A};
R⁴ is selected from halogen, C₁₋₆alkyl, C₁₋₆haloalkyl, and —OR^{4A};
R⁵ is selected from H, CN, C₁₋₆haloalkyl, 3-10 membered heterocycloalkyl;
R⁶ and R⁷ are H or R⁶ and R⁷ together form a C₃₋₇cycloalkyl;
M is selected from —CH₂— and —CF₂—;
W is selected from —O— and —NH—;
Q, U, and V are independently selected from N, O, S, CR^{Q}, C(R^{Q})₂, NR^{V}, N(R^{V})₂⁺, C═CH₂, and C═O;
each R^{Q} is independently selected from H, halogen, —OR^{Q2}, CN, C₁₋₆alkyl, C₁₋₆haloalkyl, C₂₋₆alkenyl, C₃₋₇cycloalkyl, 3-7 membered heterocycloalkyl, —NHR^{Q2}, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, and 5-10 membered heteroaryl of R^{Q} are each optionally substituted with 1 or 2 R⁹ groups;
each R^{V} is independently selected from H, C₁₋₆alkyl, C₁₋₆haloalkyl, C₃₋₇cycloalkyl, 4-7 membered heterocycloalkyl, 6-10 membered aryl, and —(C═O)OR^{V1}; wherein each C₁₋₆alkyl, C₁₋₆ haloalkyl, C₃₋₇cycloalkyl, and 4-7 membered heterocycloalkyl of R^{V} are each optionally substituted with 1 or 2 R¹⁰ groups;
each R^{Q2} is independently selected from H, C₁₋₆alkyl, 6-10 membered aryl, and 5-10 membered heteroaryl; wherein the 6-10 membered aryl, and 5-10 membered heteroaryl of R^{Q2} is optionally substituted with 1 or 2 R¹¹ groups;
each R⁹ and R¹⁰ are independently selected from halogen, —OR^{X2}, CN, C₁₋₆alkyl, C₃₋₇ cycloalkyl, 6-10 membered aryl, 5-10 membered heteroaryl, and —(C═O)NR^{X2}; wherein the C₃₋₇cycloalkyl, 6-10 membered aryl, and 5-10 membered heteroaryl of R⁹ and R¹⁰ are each optionally substituted with 1 or 2 R¹² groups;
each R¹¹ and R¹² are independently selected from —OR^{X3}, and C₁₋₆alkyl; and
each R^{3A}, R^{4A}, R^{V1}, R^{X2} and R^{X3} are independently selected from H and C₁₋₆alkyl.

Some embodiments provide a compound of Formula (IIa), or a pharmaceutically acceptable salt thereof,

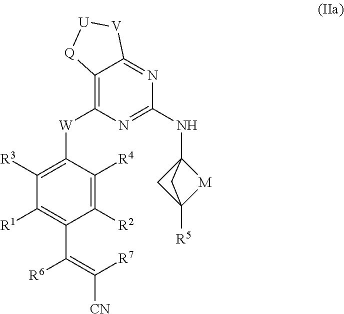

wherein [symbol] is a single bond or a double bond; and R¹, R², R³, R⁴, R⁵, R⁶, R⁷, M, W, Q, U, and V are defined herein.

In some embodiments of Formula (IIa), both R¹ and R² are H. In some embodiments, both R³ and R⁴ are methyl. In some embodiments, R⁵ is CN. In some embodiments, both R⁶ and R⁷ are H.

In some embodiments, both R¹ and R² are H, both R³ and R⁴ are methyl, R⁵ is CN, both R⁶ and R⁷ are H, and W is O.

Some embodiments provide a compound of Formula selected from (IIb), (IIc), (IId), (IIe), (IIf), (IIg), (IIh), (IIi), and (IIj), or a pharmaceutically acceptable salt thereof,

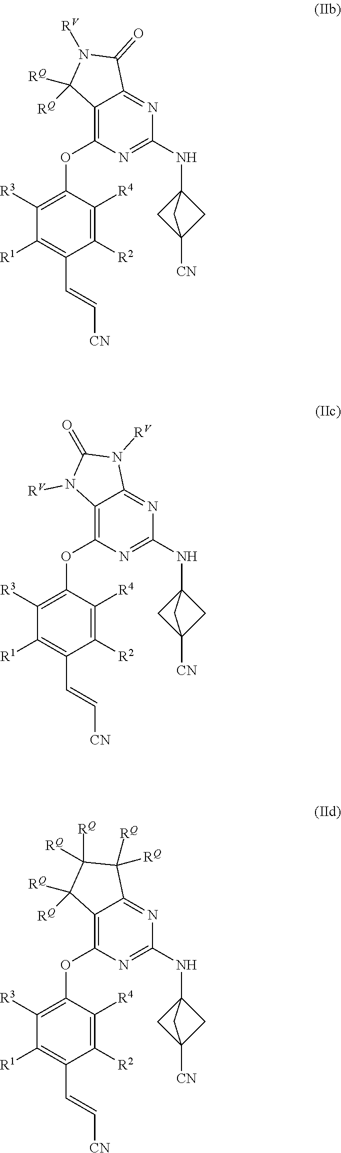

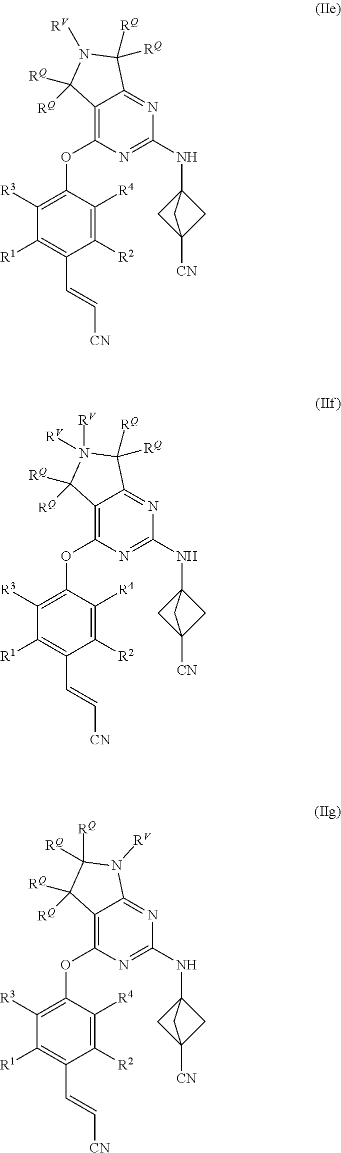

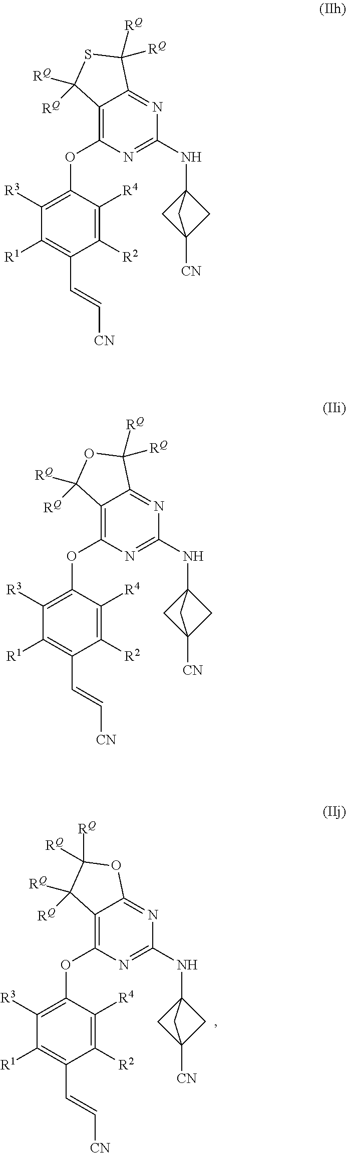

wherein R¹, R², R³, R⁴, R^{V}, and R^{Q} are defined herein.

Some embodiments provide a compound of Formula (IIb), or a pharmaceutically acceptable salt thereof,

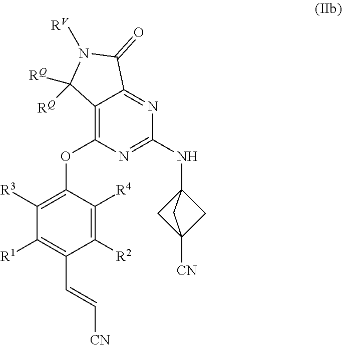

wherein R¹, R², R³, R⁴, R^{V}, and R^{Q} are defined herein.

In some embodiments of Formula (IIb), both R¹ and R² are H.

In some embodiments of Formula (IIb), each R³ and R⁴ is independently selected from halogen, C₁₋₆alkyl, C₁₋₆haloalkyl, and —OR^{3A}. In some embodiments, each R³ and R⁴is independently selected from F, Cl, methyl, ethyl, methoxyl, and ethoxyl. In some embodiments, both R³ and R⁴ are methyl.

In some embodiments of Formula (IIb), R^{V} is selected from H, C₁₋₆alkyl, C₁₋₆haloalkyl, C₃₋₇cycloalkyl, 4-7 membered heterocycloalkyl, 6-10 membered aryl, and 5-10 membered heteroaryl. In some embodiments of Formula (IIb), R^{V} is selected from H, methyl, ethyl, —CHCHF₂, —CH₂F, —CHF₂, and —CF₃. In some embodiments of Formula (IIb), R^{V} is selected from H, cyclopropyl, and —CHCHF₂.

In some embodiments of Formula (IIb), each R^{Q} is independently selected from H, halogen, —OR^{Q2}, C₁₋₆alkyl, C₁₋₆haloalkyl, and C₃₋₇cycloalkyl. In some embodiments, each R^{Q} is independently selected from H, methyl, —OH, —CH₂F, —CHF₂, and —CF₃.

Some embodiments provide a compound of Formula (IIc), or a pharmaceutically acceptable salt thereof,

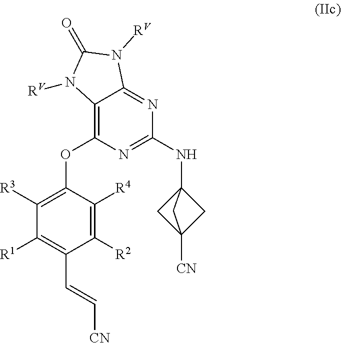

wherein R¹, R², R³, R⁴, and R^{V} are defined herein.

In some embodiments of Formula (IIc), both R¹ and R² are H.

In some embodiments of Formula (IIc), both R³ and R⁴ are methyl.

In some embodiments of Formula (IIc), each R^{V} is independently selected from H, C₁₋₆alkyl, C₁₋₆haloalkyl, and C₃₋₇cycloalkyl. In some embodiments, each R^{V} is independently selected from H, methyl, ethyl, —CH₂F, —CHF₂, and —CF₃.

Some embodiments provide a compound of Formula (IId), or a pharmaceutically acceptable salt thereof,

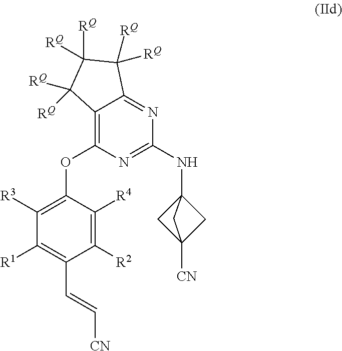

wherein R¹, R², R³, R⁴, and R^{Q} are defined herein.

In some embodiments of Formula (IId), both R¹ and R² are H.

In some embodiments of Formula (IId), both R³ and R⁴ are methyl.

In some embodiments of Formula (IId), each R^{Q} is independently selected from H, halogen, —OR^{Q2}, C₁₋₆alkyl, C₁₋₆haloalkyl, and C₃₋₇cycloalkyl. In some embodiments, each R^{Q} is independently selected from H, F, Cl, and methyl.

Some embodiments provide a compound of Formula (IIe), or a pharmaceutically acceptable salt thereof,

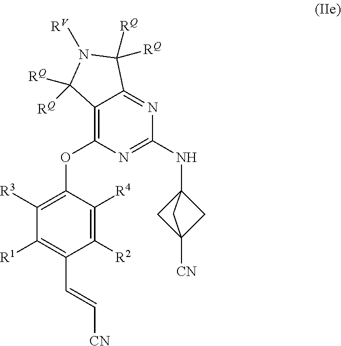

wherein R¹, R², R³, R⁴, R^{V}, and R^{Q} are defined herein.

In some embodiments of Formula (IIe), both R¹ and R² are H.

In some embodiments of Formula (IIe), both R³ and R⁴ are methyl.

In some embodiments of Formula (IIe), R^{V} is selected from H, C₁₋₆alkyl, C₁₋₆haloalkyl, and —(C═O)OR^{V1}. In some embodiments, R^{V} is selected from H, methyl, —C(O)OCH₃, and —C(O)OC(CH₃)₃.

In some embodiments of Formula (IIe), each R^{Q} is independently selected from H, halogen, —OR^{Q2}, C₁₋₆alkyl, C₁₋₆haloalkyl, and C₃₋₇cycloalkyl. In some embodiments, each R^{Q} is independently selected from H, F, and methyl.

Some embodiments provide a compound of Formula (IIf), or a pharmaceutically acceptable salt thereof,

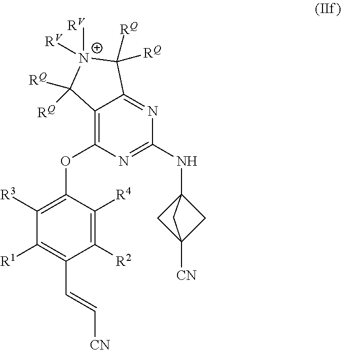

wherein R¹, R², R³, R⁴, R^{V}, and R^{Q} are defined herein.

In some embodiments of Formula (IIf), both R¹ and R² are H.

In some embodiments of Formula (IIf), both R³ and R⁴ are methyl.

In some embodiments of Formula (IIf), each R^{V} is independently selected from C₁₋₆alkyl, and C₁₋₆haloalkyl. In some embodiments, each R^{V} is methyl.

In some embodiments of Formula (IIf), each R^{Q} is independently selected from H, halogen, C₁₋₆alkyl, and C₁₋₆haloalkyl. In some embodiments, each R^{Q} is independently selected from H, and methyl.

Some embodiments provide a compound of Formula (IIg), or a pharmaceutically acceptable salt thereof,

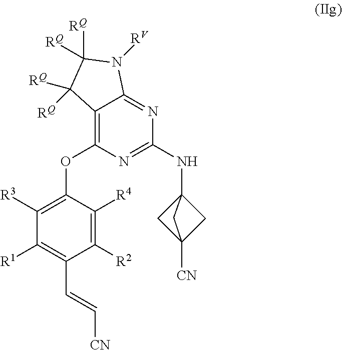

wherein R¹, R², R³, R⁴, R^{V}, and R^{Q} are defined herein.

In some embodiments of Formula (IIg), both R¹ and R² are H.

In some embodiments of Formula (IIg), both R³ and R⁴ are methyl.

In some embodiments of Formula (IIg), R^{V} is selected from H, C₁₋₆alkyl, and C₁₋₆haloalkyl. In some embodiments, R^{V} is H.

In some embodiments of Formula (IIg), each R^{Q} is independently selected from H, halogen, C₁₋₆alkyl, and C₁₋₆haloalkyl. In some embodiments, each R^{Q} is independently selected from H, and methyl.

Some embodiments provide a compound of Formula (IIh), or a pharmaceutically acceptable salt thereof,

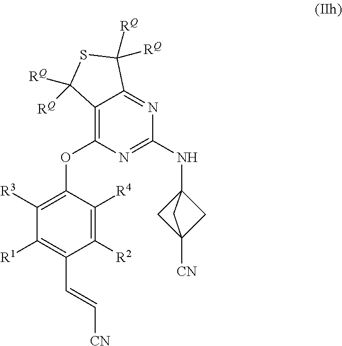

wherein R¹, R², R³, R⁴, and R^{Q} are defined herein.

In some embodiments of Formula (IIh), both R¹ and R² are H.

In some embodiments of Formula (IIh), both R³ and R⁴ are methyl.

In some embodiments of Formula (IIh), each R^{Q} is independently selected from H, halogen, C₁₋₆alkyl, and C₁₋₆haloalkyl. In some embodiments, each R^{Q} is independently selected from H, and methyl.

Some embodiments provide a compound of Formula (IIi), or a pharmaceutically acceptable salt thereof,

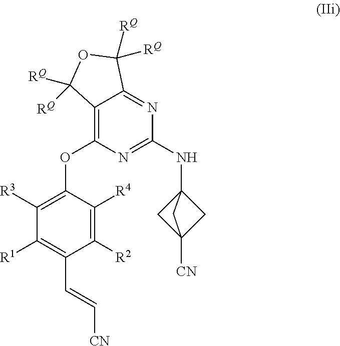

wherein R¹, R², R³, R⁴, and R^{Q} are defined herein.

In some embodiments of Formula (IIi), both R¹ and R² are H.

In some embodiments of Formula (IIi), both R³ and R⁴ are methyl.

In some embodiments of Formula (IIi), each R^{Q} is independently selected from H, halogen, C₁₋₆alkyl, and C₁₋₆haloalkyl. In some embodiments, each R^{Q} is independently selected from H, and methyl.

Some embodiments provide a compound of Formula (IIj), or a pharmaceutically acceptable salt thereof,

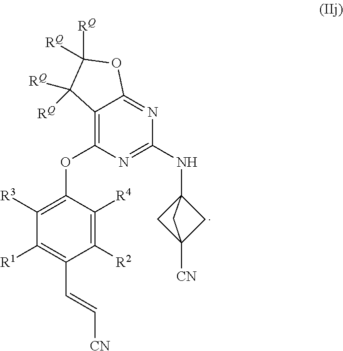

In some embodiments of Formula (IIj), both R¹ and R² are H.

In some embodiments of Formula (IIj), both R³ and R⁴ are methyl.

In some embodiments of Formula (IIj), each R^{Q} is independently selected from H, halogen, C₁₋₆alkyl, and C₁₋₆haloalkyl. In some embodiments, each R^{Q} is independently selected from H, and methyl.

Some embodiments provide a compound of Formula (IIIa), or a pharmaceutically acceptable salt thereof,

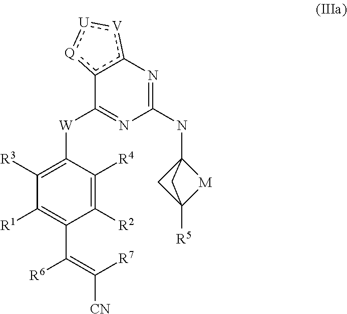

wherein [symbol] is a single bond or a double bond; and R¹, R², R³, R⁴, R⁵, R⁶, R⁷, M, W, Q, U, and V are defined herein.

In some embodiments of Formula (IIIa), both R¹ and R² are H.

In some embodiments of Formula (IIIa), both R³ and R⁴ are methyl.

In some embodiments of Formula (IIIa), W is O.

Some embodiments provide a compound of Formula selected from (IIIb), (IIIc), (IIId), (IIIe), (IIIf), (IIIg), (IIIh), (IIIi), (IIIj), (IIIk), (IIIl), (IIIm), and (IIIn), or a pharmaceutically acceptable salt thereof,

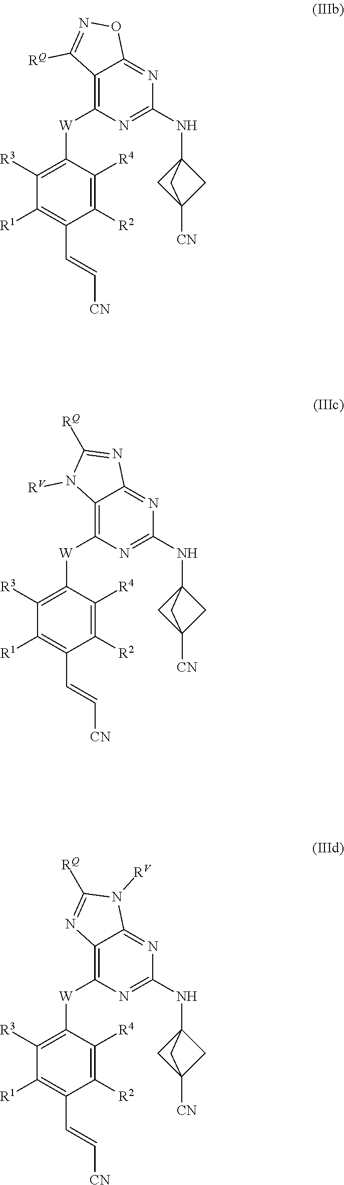

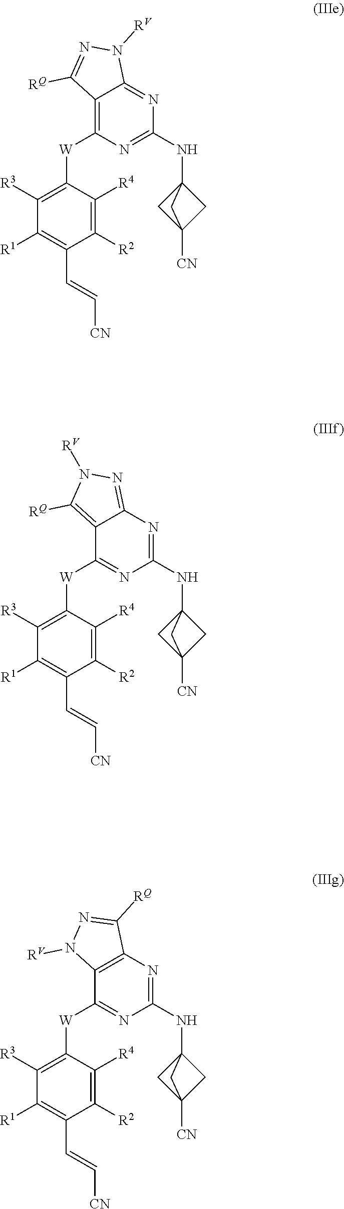

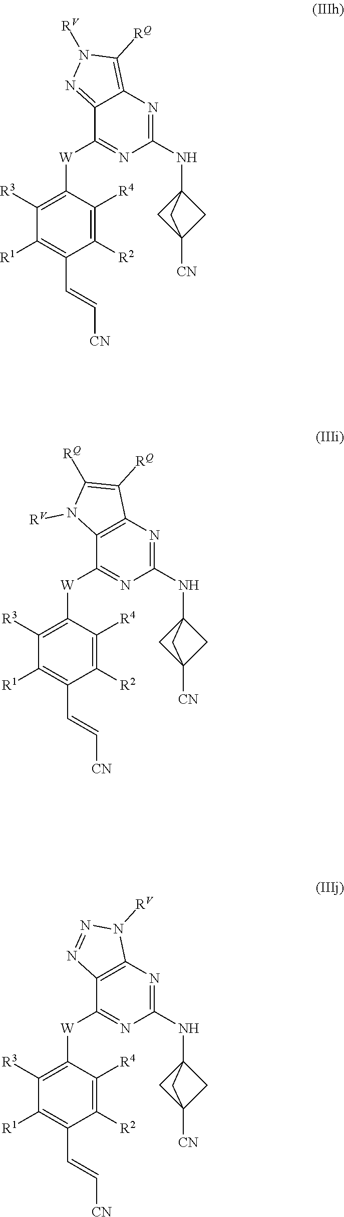

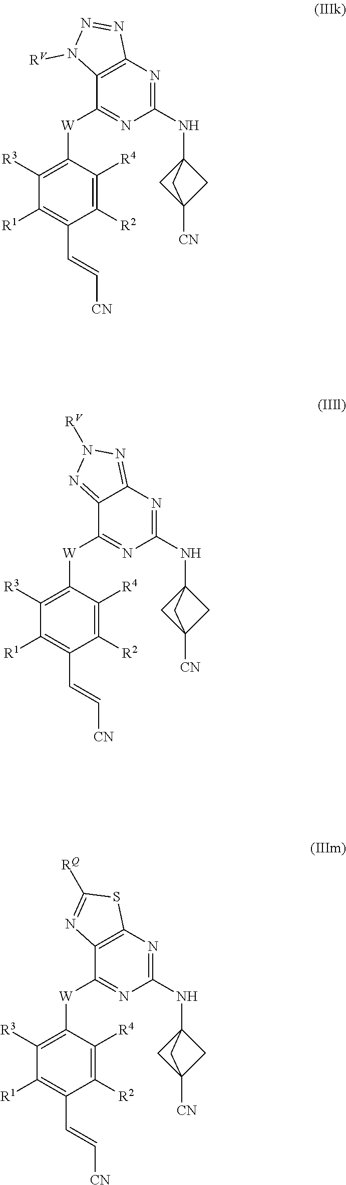

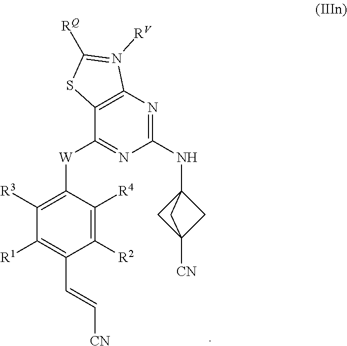

wherein R¹, R², R³, R⁴, R^{V}, and R^{Q} are defined herein.

Some embodiments provide a compound of Formula (IIIb), or a pharmaceutically acceptable salt thereof,

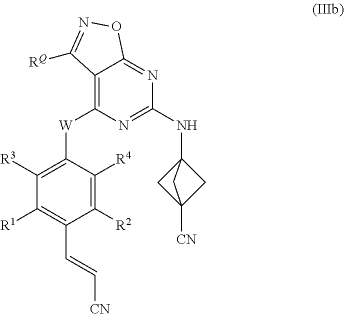

wherein R¹, R², R³, R⁴, and R^{Q} are defined herein.

In some embodiments of Formula (IIIb), both R¹ and R² are H.

In some embodiments of Formula (IIIb), both R³ and R⁴ are methyl.

In some embodiments of Formula (IIIb), W is O.

In some embodiments of Formula (IIIb), R^{Q} is selected from H, halogen, C₁₋₆alkyl, and C₁₋₆haloalkyl. In some embodiments, R^{Q} is selected from H, methyl, —CH₂F, —CHF₂, and —CF₃.

Some embodiments provide a compound of Formula (IIIc), or a pharmaceutically acceptable salt thereof,

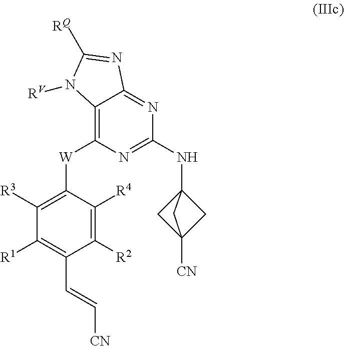

wherein R¹, R², R³, R⁴, R^{V}, and R^{Q} are defined herein.

In some embodiments of Formula (IIIc), both R¹ and R² are H.

In some embodiments of Formula (IIIc), both R³ and R⁴ are methyl.

In some embodiments of Formula (IIIc), W is O.

In some embodiments of Formula (IIIc), R^{Q} is selected from H, halogen, C₁₋₆alkyl, C₁₋₆haloalkyl, and —OC₁₋₆alkyl, wherein C₁₋₆alkyl is optional substituted with one or two groups selected from OH, and —OC₁₋₆alkyl. In some embodiments, R^{Q} is selected from H, methyl, ethyl, —CH₂F, —CHF₂, —CF₃, —OCH₃, —OCH₂CH₃, —CH₂OH, —CH(OH)CH₃, and —CH₂OCH₃.

In some embodiments of Formula (IIIc), R^{V} is selected from H, C₁₋₆alkyl, and C₁₋₆ haloalkyl. In some embodiments, R^{V} is selected from H, methyl, ethyl, —CH₂F, —CHF₂, and —CF₃. In some embodiments, R^{V} is —CHF₂.

In some embodiments of Formula (IIIc), the compound is of formula (IIIcc):

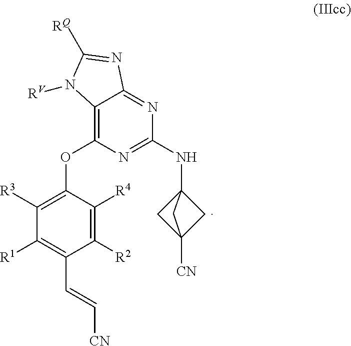

In some embodiments of Formula (IIIcc), both R¹ and R² are H.

In some embodiments of Formula (IIIcc), both R³ and R⁴ are methyl.

In some embodiments of Formula (IIIcc), R^{Q} is selected from H, halogen, C₁₋₆alkyl, C₁₋₆haloalkyl, and —OC₁₋₆alkyl, wherein C₁₋₆alkyl is optional substituted with one or two groups selected from OH, and —OC₁₋₆alkyl. In some embodiments, R^{Q} is selected from H, methyl, ethyl, —CH₂F, —CHF₂, —CF₃, —OCH₃, —OCH₂CH₃, —CH₂OH, —CH(OH)CH₃, and —CH₂OCH₃.

In some embodiments of Formula (IIIcc), R^{V} is selected from H, C₁₋₆alkyl, and C₁₋₆ haloalkyl. In some embodiments, R^{V} is selected from H, methyl, ethyl, —CH₂F, —CHF₂, and —CF₃. In some embodiments, R^{V} is —CHF₂.

Some embodiments provide a compound of Formula (IIId), or a pharmaceutically acceptable salt thereof,

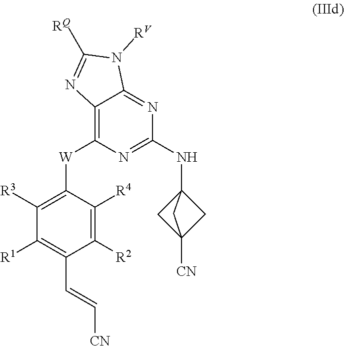

wherein R¹, R², R³, R⁴, R^{V}, and R^{Q} are defined herein.

In some embodiments of Formula (IIId), both R¹ and R² are H.

In some embodiments of Formula (IIId), both R³ and R⁴ are methyl.

In some embodiments of Formula (IIId), W is O.

In some embodiments of Formula (IIId), R^{Q} is selected from H, C₁₋₆alkyl, and C₁₋₆ haloalkyl. In some embodiments, R^{Q} is selected from H, methyl, and ethyl.

In some embodiments of Formula (IIId), R^{V} is selected from H, C₁₋₆alkyl, and C₁₋₆haloalkyl.

Some embodiments provide a compound of Formula (IIIe), or a pharmaceutically acceptable salt thereof,

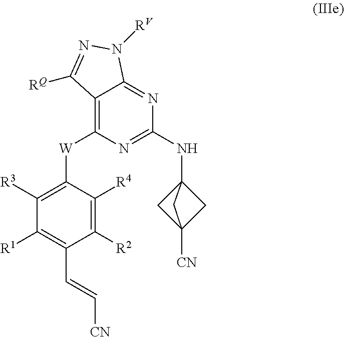

wherein R¹, R², R³, R⁴, R^{V}, and R^{Q} are defined herein.

In some embodiments of Formula (IIIe), both R¹ and R² are H.

In some embodiments of Formula (IIIe), both R³ and R⁴ are methyl.

In some embodiments of Formula (IIIe), W is O.

In some embodiments of Formula (IIIe), R^{Q} is selected from H, halogen, C₁₋₆alkyl, C₁₋₆ haloalkyl, and NH₂. In some embodiments, R^{Q} is selected from H, methyl, ethyl, Cl, Br, and NH₂.

In some embodiments of Formula (IIIe), R^{V} is selected from H, C₁₋₆alkyl, and C₁₋₆haloalkyl.

Some embodiments provide a compound of Formula (IIIf), or a pharmaceutically acceptable salt thereof,

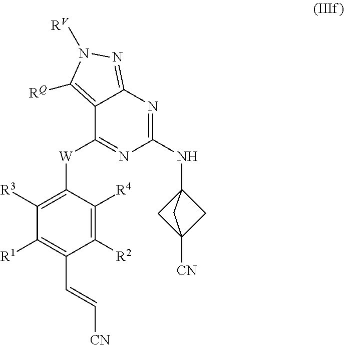

wherein R¹, R², R³, R⁴, R^{V}, and R^{Q} are defined herein.

In some embodiments of Formula (IIIf), both R¹ and R² are H.

In some embodiments of Formula (IIIf), both R³ and R⁴ are methyl.

In some embodiments of Formula (IIIf), W is O.

In some embodiments of Formula (IIIf), R^{Q} is selected from H, C₁₋₆alkyl, and C₁₋₆ haloalkyl. In some embodiments, R^{Q} is selected from H, methyl, and ethyl.

In some embodiments of Formula (IIIf), R^{V} is selected from H, C₁₋₆alkyl, and C₁₋₆haloalkyl.

Some embodiments provide a compound of Formula (IIIg), or a pharmaceutically

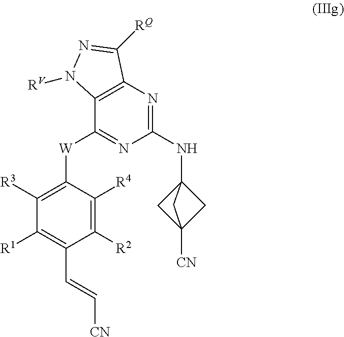

wherein R¹, R², R³, R⁴, R^{V}, and R^{Q} are defined herein.

In some embodiments of Formula (IIIg), both R¹ and R² are H.

In some embodiments of Formula (IIIg), both R³ and R⁴ are methyl.

In some embodiments of Formula (IIIg), W is O.

In some embodiments of Formula (IIIg), R^{Q} is selected from H, C₁₋₆alkyl, and C₁₋₆ haloalkyl. In some embodiments, R^{Q} is selected from H, methyl, and ethyl.

In some embodiments of Formula (IIIg), R^{V} is selected from H, C₁₋₆alkyl, and C₁₋₆haloalkyl.

Some embodiments provide a compound of Formula (IIIh), or a pharmaceutically acceptable salt thereof,

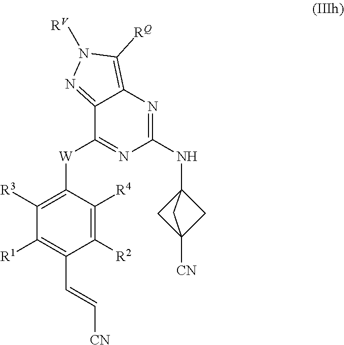

wherein R¹, R², R³, R⁴, R^{V}, and R^{Q} are defined herein.

In some embodiments of Formula (IIIh), both R¹ and R² are H.

In some embodiments of Formula (IIIh), both R³ and R⁴ are methyl.

In some embodiments of Formula (IIIh), W is O.

In some embodiments of Formula (IIIh), R^{Q} is selected from H, C₁₋₆alkyl, and C₁₋₆ haloalkyl. In some embodiments, R^{Q} is selected from H, methyl, and ethyl.

In some embodiments of Formula (IIIh), R^{V} is selected from H, C₁₋₆alkyl, and C₁₋₆haloalkyl.

Some embodiments provide a compound of Formula (IIIi), or a pharmaceutically

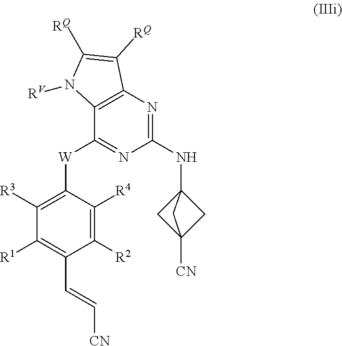

wherein R¹, R², R³, R⁴, R^{V}, and R^{Q} are defined herein.

In some embodiments of Formula (IIIi), both R¹ and R² are H.

In some embodiments of Formula (IIIi), both R³ and R⁴ are methyl.

In some embodiments of Formula (IIIi), W is O.

In some embodiments of Formula (IIIi), each R^{Q} is independently selected from H, C₁₋₆ alkyl, and C₁₋₆haloalkyl. In some embodiments, each R^{Q} is independently selected from H, methyl, and ethyl.

In some embodiments of Formula (IIIi), R^{V} is selected from H, C₁₋₆alkyl, and C₁₋₆haloalkyl.

Some embodiments provide a compound of Formula (IIIj), or a pharmaceutically acceptable salt thereof,

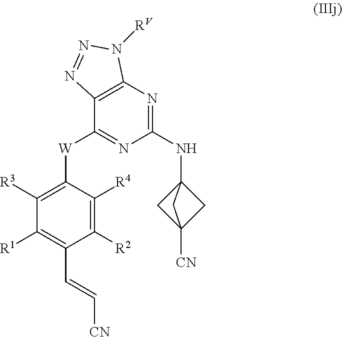

wherein R¹, R², R³, R⁴, and R^{V} are defined herein.

In some embodiments of Formula (IIIj), both R¹ and R² are H.

In some embodiments of Formula (IIIj), both R³ and R⁴ are methyl.

In some embodiments of Formula (IIIj), W is O.

In some embodiments of Formula (IIIj), R^{V} is selected from H, C₁₋₆alkyl, and C₁₋₆haloalkyl.

Some embodiments provide a compound of Formula (IIIk), or a pharmaceutically acceptable salt thereof,

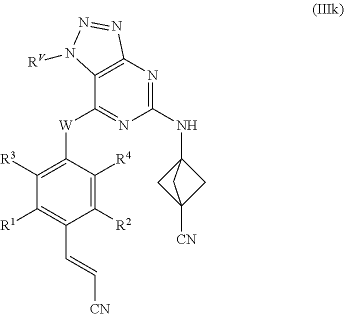

wherein R¹, R², R³, R⁴, and R^{V} are defined herein.

In some embodiments of Formula (IIIk), both R¹ and R² are H.

In some embodiments of Formula (IIIk), both R³ and R⁴ are methyl.

In some embodiments of Formula (IIIk), W is O.

In some embodiments of Formula (IIIk), R^{V} is selected from H, C₁₋₆alkyl, and C₁₋₆haloalkyl.

Some embodiments provide a compound of Formula (IIIl), or a pharmaceutically acceptable salt thereof,

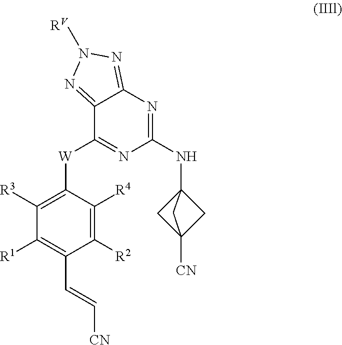

wherein R¹, R², R³, R⁴, and R^{V} are defined herein.

In some embodiments of Formula (IIIl), both R¹ and R² are H.

In some embodiments of Formula (IIIl), both R³ and R⁴ are methyl.

In some embodiments of Formula (IIIl), W is O.

In some embodiments of Formula (IIIl), R^{V} is selected from H, C₁₋₆alkyl, and C₁₋₆haloalkyl.

Some embodiments provide a compound of Formula (IIIm), or a pharmaceutically acceptable salt thereof,

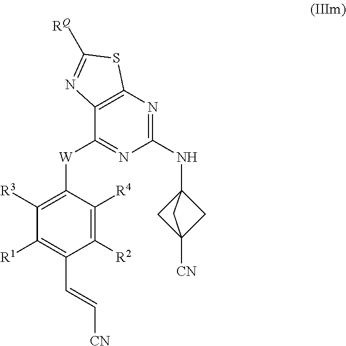

wherein R¹, R², R³, R⁴, and R^{Q} are defined herein.

In some embodiments of Formula (IIIm), both R¹ and R² are H.

In some embodiments of Formula (IIIm), both R³ and R⁴ are methyl.

In some embodiments of Formula (IIIm), W is O.

In some embodiments of Formula (IIIm), R^{Q} is selected from H, C₁₋₆alkyl, and C₁₋₆haloalkyl.

Some embodiments provide a compound of Formula (IIIn), or a pharmaceutically acceptable salt thereof,

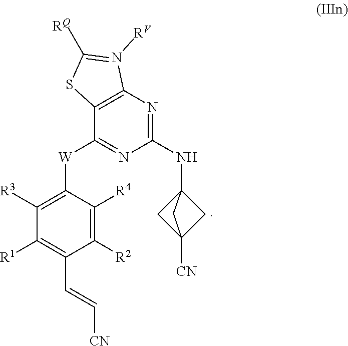

wherein R¹, R², R³, R⁴, R^{Q}, and R^{V} are defined herein.

In some embodiments of Formula (IIIn), both R¹ and R² are H.

In some embodiments of Formula (IIIn), both R³ and R⁴ are methyl.

In some embodiments of Formula (IIIn), W is O.

In some embodiments of Formula (IIIn), R^{V} is selected from H, C₁₋₆alkyl, and C₁₋₆haloalkyl.

### Pharmaceutical Compositions

Pharmaceutical compositions comprising the compounds disclosed herein, or pharmaceutically acceptable salts thereof, may be prepared with conventional carriers (e.g., inactive ingredient or excipient material) which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 5th edition, American Pharmacists Association, 1986. Excipients can include ascorbic acid and other antioxidants, chelating agents such as EDTA, carbohydrates such as dextrin, hydroxyalkylcellulose, hydroxyalkylmethylcellulose, stearic acid and the like. In certain embodiments, the composition relates to a solid dosage form, including a solid oral dosage form. The pH of a composition may range from about 3 to about 11, but is ordinarily about 7 to 10.

While it is possible for the active ingredients to be administered alone, it may be preferable to present them as pharmaceutical compositions. The compositions, both for veterinary and for human use, comprise at least one compound disclosed herein, together with one or more acceptable carriers and optionally other therapeutic ingredients. In one embodiment, the pharmaceutical composition comprises a compound disclosed herein, or a tautomer or a pharmaceutically acceptable salt thereof, a pharmaceutically acceptable carrier and one other therapeutic ingredient. The carrier(s) are “acceptable” in the sense of being compatible with the other ingredients of the composition and physiologically innocuous to the recipient thereof.

The compositions include those suitable for various administration routes, including oral administration. The compositions may conveniently be presented in unit dosage form and may be prepared by any of the methods well known in the art of pharmacy. Such methods include the step of bringing into association the active, ingredient (e.g., a compound disclosed herein disclosed herein or a pharmaceutical salt thereof) with one or more inactive ingredients (e.g., a carrier, pharmaceutical excipient, etc.). The compositions may be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21st Edition, Lippincott Wiliams and Wilkins, Philadelphia, Pa., 2006.

Compositions described herein that are suitable for oral administration may be presented as discrete units (a unit dosage form) including but not limited to capsules, cachets or tablets each containing a predetermined amount of the active ingredient.

Pharmaceutical compositions disclosed herein comprise one or more compounds disclosed herein, or a pharmaceutically acceptable salt thereof, together with one or more pharmaceutically acceptable carriers or excipients and optionally other therapeutic agents. Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 to 1000 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the compositions of these embodiments may include other agents conventional in the art having regard to the type of composition in question, for example those suitable for oral administration may include flavoring agents.

In certain embodiments, a composition comprising an active ingredient disclosed herein (a compound disclosed herein or a pharmaceutically acceptable salt thereof) in one variation does not contain an agent that affects the rate at which the active ingredient is metabolized. Thus, it is understood that compositions comprising a compound disclosed herein in certain embodiments do not comprise an agent that would affect (e.g., slow, hinder or retard) the metabolism of a compound disclosed herein or any other active ingredient administered separately, sequentially or simultaneously with a compound disclosed herein. It is also understood that any of the methods, kits, articles of manufacture and the like detailed herein in certain embodiments do not comprise an agent that would affect (e.g., slow, hinder or retard) the metabolism of a compound disclosed herein or any other active ingredient administered separately, sequentially or simultaneously with a compound of any one disclosed herein.

### Methods of Use

Disclosed herein is a method of prematurely activating an HIV protease in an individual in need thereof, comprising administering a compound disclosed herein, or a pharmaceutically acceptable salt thereof, to the individual. Disclosed herein is a method of inhibiting an HIV reverse transcriptase in an individual in need thereof, comprising administering a compound disclosed herein, or a pharmaceutically acceptable salt thereof, to the individual. In certain embodiments, the individual in need thereof is a human who has been infected with HV. In certain embodiments, the individual in need thereof is a human who has been infected with MV but who has not developed AIDS. In certain embodiments, the individual in need thereof is an individual at risk for developing AIDS. In certain embodiments, the individual in need thereof is a human who has been infected with HIV and who has developed AIDS. In certain embodiments of the methods disclosed herein, a compound disclosed herein, or a pharmaceutically acceptable salt thereof, is administered to the individual separately, sequentially or simultaneously with another active ingredient for treating HIV, such as, HV non-nucleoside inhibitors of reverse transcriptase, HV nucleoside inhibitors of reverse transcriptase, HV nucleotide inhibitors of reverse transcriptase, HIV integrase inhibitors, HIV Tat inhibitors, HIV Tat mimetics, gp41 inhibitors, CXCR4 inhibitors, gp120 inhibitors, CCR5 inhibitors, capsid polymerization inhibitors, and other drugs for treating HIV, and combinations thereof.

In certain embodiments, a method for treating or preventing an HIV viral infection in an individual (e.g., a human), comprising administering a compound disclosed herein, or a pharmaceutically acceptable salt thereof, to the individual is disclosed.

In certain embodiments, a method for inhibiting the replication of the HIV virus, treating AIDS or delaying the onset of AIDS in an individual (e.g., a human), comprising administering a compound disclosed herein, or a pharmaceutically acceptable salt thereof, to the individual is disclosed.

In certain embodiments, a method for preventing an HIV infection in an individual (e.g., a human), comprising administering a compound disclosed herein, or a pharmaceutically acceptable salt thereof, to the individual is disclosed. In certain embodiments, the individual is at risk of contracting the HIV virus, such as an individual who has one or more risk factors known to be associated with contracting the HIV virus.

In certain embodiments, a method for treating an HIV infection in an individual (eg., a human), comprising administering a compound disclosed herein, or a pharmaceutically acceptable salt thereof, to the individual is disclosed.

In certain embodiments, a method for treating an HIV infection in an individual (e.g., a human), comprising administering to the individual in need thereof a therapeutically effective amount of a compound disclosed herein, or a pharmaceutically acceptable salt thereof, in combination with a therapeutically effective amount of one or more additional therapeutic agents selected from the group consisting of HIV non-nucleoside inhibitors of reverse transcriptase, HIV nucleoside inhibitors of reverse transcriptase, HIV nucleotide inhibitors of reverse transcriptase, HIV integrase inhibitors, HIV Tat inhibitors, HIV Tat mimetics, gp41 inhibitors, CXCR4 inhibitors, gp120 inhibitors, CCR5 inhibitors, capsid polymerization inhibitors, and other drugs for treating HIV, and combinations thereof is disclosed.

In certain embodiments, a compound disclosed herein, or a pharmaceutically acceptable salt thereof for use in medical therapy of an HIV viral infection (e.g. HIV-1 or the replication of the HIV virus (e.g. HIV-1) or AIDS or delaying the onset of AIDS in an individual (e.g., a human)) is disclosed.

In certain embodiments, a compound of any disclosed herein, or a pharmaceutically acceptable salt thereof for use in the manufacture of a medicament for treating an HIV viral infection or the replication of the HIV virus or AIDS or delaying the onset of AIDS in an individual (e.g., a human) is disclosed. One embodiment relates to a compound disclosed herein, or a pharmaceutically acceptable salt thereof, for use in the prophylactic or therapeutic treatment of an HIV infection or AIDS or for use in the therapeutic treatment or delaying the onset of AIDS is disclosed.

In certain embodiments, the use of a compound disclosed herein, or a pharmaceutically acceptable salt thereof, for the manufacture of a medicament for an HIV virus infection in an individual (e.g., a human) is disclosed. In certain embodiments, a compound of any disclosed herein, or a pharmaceutically acceptable salt thereof, for use in the prophylactic or therapeutic treatment of an HIV virus infection is disclosed.

In certain embodiments, in the methods of use, the administration is to an individual (e.g., a human) in need of the treatment. In certain embodiments, in the methods of use, the administration is to an individual (e.g., a human) who is at risk of developing AIDS.

Disclosed herein is a compound disclosed herein, or a pharmaceutically acceptable salt thereof, for use in therapy. In one embodiment, the compound disclosed herein, or a pharmaceutically acceptable salt thereof, is for use in a method of treating an HIV viral infection or the replication of the HIV virus or AIDS or delaying the onset of AIDS in an individual (e.g., a human).

Also disclosed herein is a compound disclosed herein, or a pharmaceutically acceptable salt thereof, for use in a method of treating or preventing HIV in an individual in need thereof. In certain embodiments, the individual in need thereof is a human who has been infected with HIV. In certain embodiments, the individual in need thereof is a human who has been infected with HIV but who has not developed AIDS. In certain embodiments, the individual in need thereof is an individual at risk for developing AIDS. In certain embodiments, the individual in need thereof is a human who has been infected with HIV and who has developed AIDS.

Also disclosed herein is a compound disclosed herein, or a pharmaceutically acceptable salt thereof, for use in the therapeutic treatment or delaying the onset of AIDS.

Also disclosed herein is a compound disclosed herein, or a pharmaceutically acceptable salt thereof, for use in the prophylactic or therapeutic treatment of an HIV infection.

In certain embodiments, a compound disclosed herein, or a pharmaceutically acceptable salt thereof can be used as a research tool (e.g. to study the premature activation of HIV protease in a subject or in vitro).

### Routes of Administration

One or more compounds disclosed herein (also referred to herein as the active ingredients) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. In certain embodiments, the compounds disclosed are orally bioavailable and can be dosed orally.

### HIV Combination Therapy

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein, or a pharmaceutically acceptable salt thereof, in combination with one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, HIV Tat mimetics, dipeptidyl peptidase IX (DPP9) inhibitors, immunomodulators, immunotherapeutic agents, antibody-drug conjugates, gene modifiers, gene editors (such as CRISPR/Cas9, zinc fingercleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), HIV latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MILK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV reverse transcriptase inhibitors, HIV integrase inhibitors, HIV non-catalytic site (or allosteric) integrase inhibitors, HIV entry (fusion) inhibitors, HIV maturation inhibitors, HIV latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV reverse transcriptase inhibitors, HIV integrase inhibitors, HIV non-catalytic site (or allosteric) integrase inhibitors, HIV entry (fusion) inhibitors, HIV maturation inhibitors, HIV latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, HIV latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir and lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; PREZCOBIX® (darunavir and cobicistat); dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, HA-722 (dolutegravir+lamivudine+tenofovir disoproxil fumarate), lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, ACC-008 (ACC-007+lamivudine+tenofovir disoproxil fumarate), VM-1500+emtricitabine+tenofovir disoproxil, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, (elsulfavirine, VM-1500), depulfavirine (VM-1500A), lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, Gamimune, metenkefalin, naltrexone, Prolastin, REP 9, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, bevirimat, ABBV-382, obefazimod (ABX-464), AG-1105, APH-0812, APH0202, bryostatin-1, bryostatin-23, bryostatin analogs, SUW-133, BIT-225, BRII-732, BRII-778, Codivir, CYT-107, CS-TATI-1, fluoro-beta-D-arabinose nucleic acid (FANA)-modified antisense oligonucleotides, FX-101, griffithsin, HGTV-43, HPH-116, HRS-5685, HivCide-I, hydroxychloroquine, INB-10035, IMO-3100, IND-02, JL-18008, LADAVRU, LLDT-8, MK-1376, MK-2048, MK-4250, ulonivirine (MK-8507), MK-8558, islatravir (MK-8591), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IMLL-106, antiviral fc conjugate (AVC), WP-1096, WP-1097, Gammora, ISR—CO48, ISR-48, ISR-49, MK-8527, cannabinoids, ENOB-HV-32, T-1144, VIR-576, nipamovir, Covimro, WP-1122, ZFP-362, MK-8510, and ABBV-1882.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, difluoro-biphenyl-diarylpyrimidines (DAPY), efavirenz, etravirine, GS-5894, lentinan, nevirapine, rilpivirine, ACC-007, ACC-018, AIC-292, F-18, KM-023, PC-1005, M1-TFV, M2-TFV, VM-1500A-LAI, PF-3450074, elsulfavirine (sustained release oral), doravirine+islatravir (fixed dose combination/oral tablet formulation), elsulfavirine (long acting injectable nanosuspension), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir amibufenamide fumarate (HS-10234), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, GSK-4023991, MK-8504, islatravir, MK-8583, VM-2500, CL-197, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, GS-1720, GS-6212, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, 5-365598, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, HIV-1 fusion inhibitors (P26-Bapc), ITV-1, ITV-2, ITV-3, ITV-4, CPT-31, C13hmAb, lipovirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, balixafortide and vMIP (Haimipu).

### HIV Maturation Inhibitors

Examples of HIV maturation inhibitors include, but are not limited to, BMS-955176, fipravirimat mesylate (GSK-3640254), VH-3739937 (GSK-3739937), HRF-10071 and GSK-2838232.

### HIV Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, LAP antagonists (inhibitor of apoptosis proteins, such as APG-1387, LBW-242), HIV Tat inhibitors, HIV Tat mimetics, SMAC mimetics (including TL32711, LCL161, GDC-0917, HGS1029, xevinapant (AT-406, Debio-1143)), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, ixazomib, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HIBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### DPP9 Inhibitors

Examples of DPP9 inhibitors include, but are not limited to, talobostat.

### HIV Tat Inhibitors

Examples of Tat inhibitors include, but are not limited to, CS-TATI-1.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-4182, GS-CA1, AVI-621, AVI-101, AVI-201, AVI-301, and AVI-CAN1-15 series, PF-3450074, VH-4004280, VH-4011499, and compounds described in (GSK WO2019/087016).

Additional examples of capsid inhibitors include, but not limited to, those described in U.S. Patent Application Publication Nos. US2018051005 and US2016108030.

### Cytochrome P450 3A Inhibitors

Examples of Cytochrome P450 3A inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TEVID4; TIM4); hepatitis A virus cellular receptor 2 (HAVCR2, TEVID3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301,envafolimab (ASC-22, KN-035), PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, budigalimab (ABBV-181), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, SHR-2150, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as nogapendekin alfa (ALT-803), NKTR-255, and hetIL-15, interleukin-15/Fc fusion protein, AM-0015, NIZ-985, SO—C101, IL-15 Synthorin (pegylated IL15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, and peginterferon alfa-2b.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4/beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, Immuglo, MB-66, clone 3 human monoclonal antibody targeting KLIC (HIV infection), GS-9721, teropavimab (GS-5423), teropavimab (GS-2872), BG-HIV, VRC-HIIVMAB091-00-AB. Anti-CD4 antibodies such as ibalizumab, TMB-365, and MGD-020.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 132530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):D1 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):D1 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, GSK-3810109 (N6LS), N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM-1400LS, PGDM12, PGDM21, PCDN-33A, 2Dm2m, 4Dm2m, 6Dm2m, PGDM1400, MDX010 (ipilimumab), VRC01, VRC-01-LS, VRC01-23LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation GS-8588, MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AdC6-HIVgp140, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAd0x1.tHIVconsv1 vaccine, CMV-MVA triplex vaccine, ChAd0x1.HTI, C62-M4, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIIV-PT4, DNA-HIV-PT123, rAAV1-PG9DP, GOVX-B11, GOVX-B21, GOVX-C55, GS-1983, GS-6708, TVI-HIV-1, Ad-4 (Ad4-env Clade C+Ad4-mGag), Paxvax, EN41-UGR7C, EN41-FPA2, ENOB-HV-11, ENOB-HV-12, exoVACC, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, UVAX-1107, repRNA vaccine (LION nanoparticle, HIV-1), 763SIP8/MPLA-5 vaccine, BG505 SOSIP.664 gp140 adjuvanted vaccine, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, MVA.tHIVconsv3, MVA.tHIVconsv4, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based vaccines, VPI-211, HIV ANTI-CD40.ENV GP140, HIV ANTI-CD40.HIV5PEP, multimeric HIV gp120 vaccine TBL-1203HI, CH505 TF chTrimer, CD40.HIVRI.Env vaccine, VRC-HIVRGP096-00-VP, Drep-HIV-PT-1, BG505 MD39.3 mRNA, BG505 MD39.3 gp151 CD4KO mRNA, BG505 MD39.3 gp151 mRNA, mRNA-1644, mRNA-1547, and mRNA-1574, and anti-HIV vaccines described in WO2021011544 and WO2022155258.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; EDURANT® (dolutegravir+rilpivirine); raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101, XVIR-TAT.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-Env duoCAR T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells.

In certain embodiments, the agents described herein are combined with IMC-M113V, a TCR bispecific having a TCR binding domain that targets a peptide derived from the Gag protein presented by HLA*A02 on the surface of HIV infected cells and a second antigen binding domain that targets CD3.

### B-Cell Therapy

In certain embodiments, the agents described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of formula I, II, III, IV, or V) may be combined with one, two, three, or four additional therapeutic agents in any dosage amount of the compound of formula I, II, III, IV, or V (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, lenacapavir, cabotegravir, rilpivirine, any integrase LA, VM-1500 LAI, GS-1614, maraviroc (LAI), tenofovir implant, islatravir, islatravir implant, islatravir prodrug, doravirine, LYN-172, raltegravir, XVIR-120, GSK-3739937 (long-acting), and long acting dolutegravir, CABENUVA® (cabotegravir LA+rilpivirine LA), VOCABRIA® (cabotegravir LA), APRETUDE® (cabotegravir LAI), REKAMBYS®(rilpivirine LA).

### Kits and Articles of Manufacture

In one embodiment, kits comprising a compound disclosed herein, or a pharmaceutically acceptable salt thereof, in combination with one or more (e.g., one, two, three, one or two, or one to three) additional therapeutic agents are provided.

The present disclosure relates to a kit comprising a compound disclosed herein, or a pharmaceutically acceptable salt thereof. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising a compound of any disclosed herein, or a pharmaceutically acceptable salt thereof. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

Also disclosed are articles of manufacture comprising a unit dosage of a compound of any disclosed herein, or a pharmaceutically acceptable salt thereof, in suitable packaging for use in the methods described herein. Suitable packaging is known in the art and includes, for example, vials, vessels, ampules, bottles, jars, flexible packaging and the like. An article of manufacture may further be sterilized and/or sealed.

### EXAMPLES

Representative syntheses of compounds of the present disclosure are described in schemes below, and the particular examples that follow. The following examples are merely illustrative, and not intended to limit this disclosure in any way. It is to be understood that individual steps described herein may be combined. It is also to be understood that separate batches of a compound may be combined and carried forth in the next synthetic step.

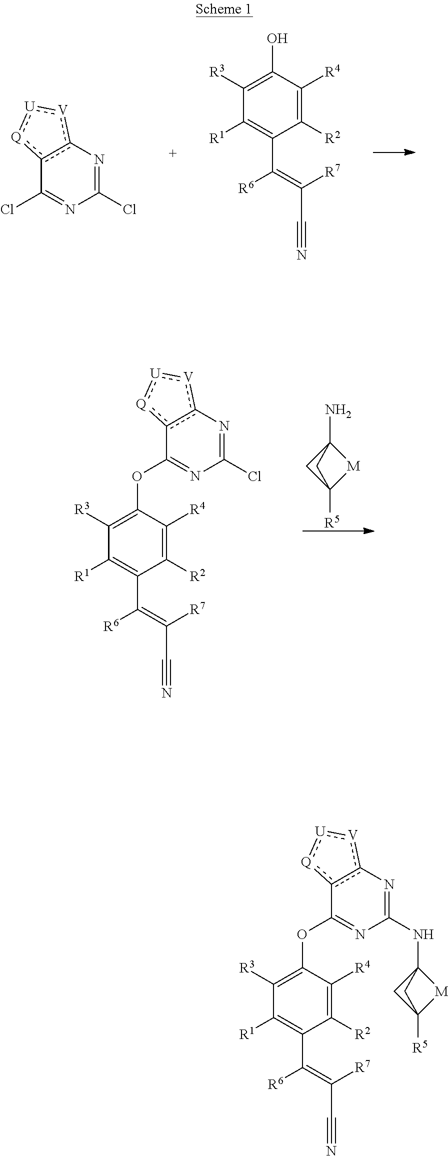

Scheme 1 shows a general synthesis of the compounds of the embodiments. The methodology is compatible with a wide variety of functionalities.

In Scheme 1, a suitably substituted heteroaryl chloride (or the corresponding bromo- or fluoro-compound) is combined with a nucleophile (e.g. aryl amine, aryl alcohol, alkyl amine etc.) in a suitable solvent system (e.g. NMP, DMF, DMAc, DMSO, acetonitrile, EtOH, THF etc.) in the presence of a base (e.g. Cs₂CO₃, K₂CO₃, triethylamine, DIPEA, NaH etc.) at ambient or elevated temperature (e.g., ranging from about 20-120° C.).

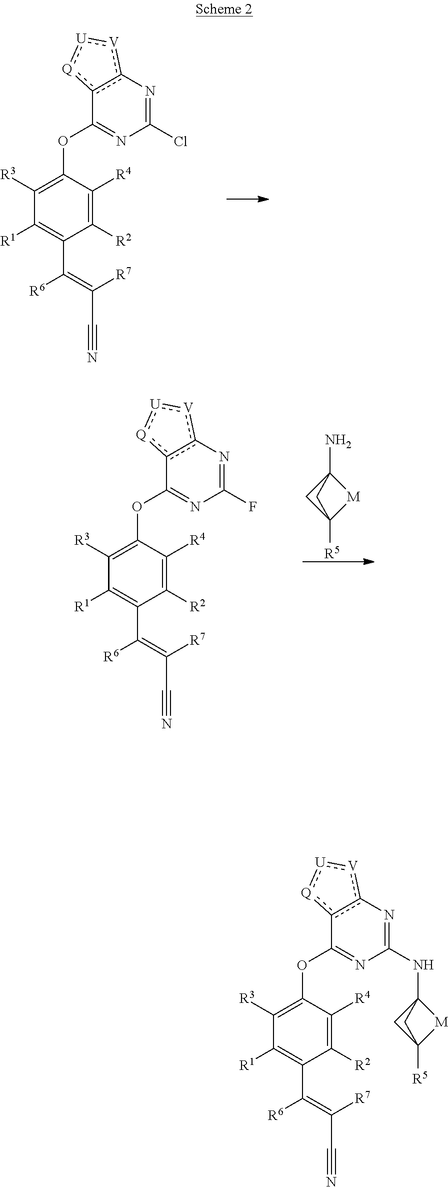

Scheme 2 shows a general synthesis of the compounds of the embodiments. The methodology is compatible with a wide variety of functionalities.

In Scheme 2, a suitably substituted heteroaryl chloride (or the corresponding bromo compound) is combined with a fluoride ion source (e.g. KF, CsF, TBAF etc.) and a nucleophilic tertiary amine (e.g. DABCO, quinuclidine etc.) in a suitable solvent system (e.g. DMF, DMSO, NMP, DMAc, tBuOH etc.) at ambient or elevated temperature (e.g., ranging from about 20-100° C.). The resulting heteroaryl fluoride is combined with a suitable alkyl amine nucleophile (e.g. 3-aminobicyclo[1.1.1]pentane-1-carbonitrile; hydrochloride) and base (e.g. K₂CO₃, Cs₂CO₃, NaH etc.) at ambient or elevated temperature (e.g., ranging from about 20-120° C.).

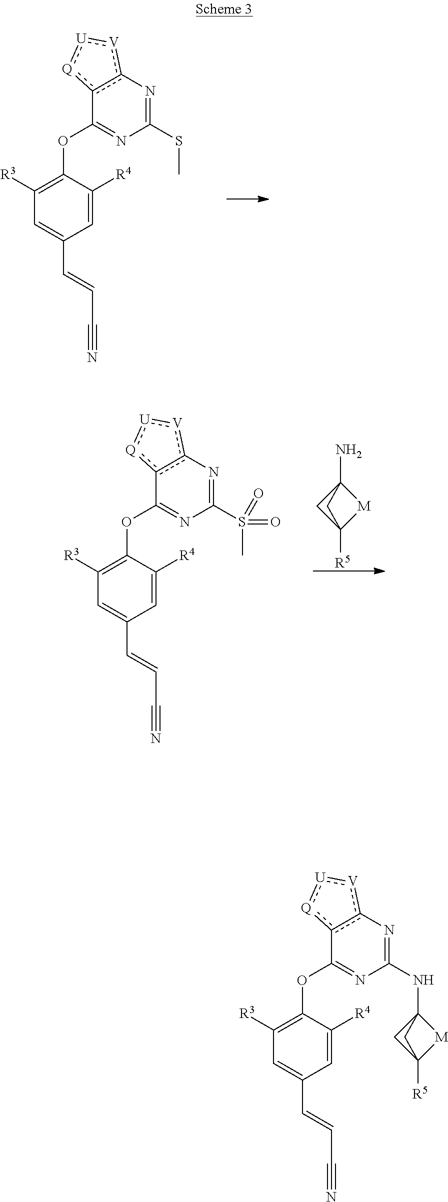

Scheme 3 shows a general synthesis of the compounds of the embodiments. The methodology is compatible with a wide variety of functionalities.

In Scheme 3, a suitably substituted heteroaryl thioether is combined with an oxidant (e.g. mCBPA, ammonium molybdate/hydrogen peroxide etc.) in a suitable solvent system (e.g. NMP, DCM, EtOH etc.) at cryogenic or ambient temperature (e.g., ranging from about 0-20° C.). The resulting heteroaryl sulfone is combined with a suitable alkyl amine nucleophile (e.g. 3-aminobicyclo[1.1.1]pentane-1-carbonitrile; hydrochloride) and base (e.g. K₂CO₃, Cs₂CO₃, triethylamine, DIPEA, NaH etc.) at ambient or elevated temperature (e.g., ranging from about 20-120° C.).

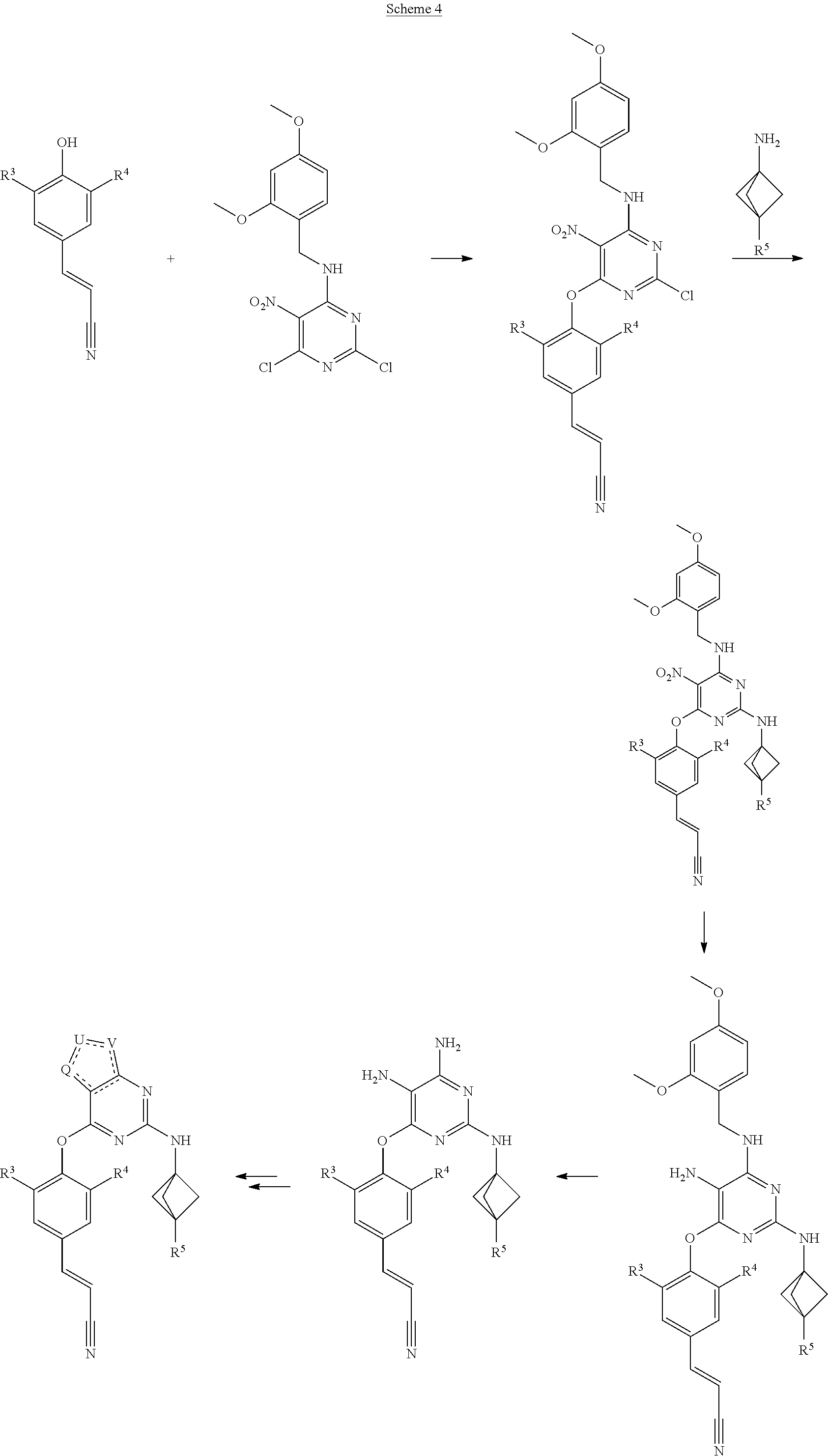

Scheme 4 shows a general synthesis of the compounds of the embodiments. The methodology is compatible with a wide variety of functionalities.

In Scheme 4, a suitably substituted heteroaryl chloride (or the corresponding bromo- or fluoro-compound) is combined with a nucleophile (e.g. aryl amine, aryl alcohol, alkyl amine etc.) in a suitable solvent system (e.g. NMP, DMF, DMAc, DMSO, acetonitrile, EtOH, THF etc.) in the presence of a base (e.g. Cs₂CO₃, K₂CO₃, triethylamine, DIPEA, NaH etc.) at ambient or elevated temperature (e.g., ranging from about 20-120° C.).

The resulting nitro-substituted heteroaryl compound is combined with a reductant (e.g. zinc, iron etc.) and acid (e.g. acetic acid, formic acid, hydrochloric acid, ammonium chloride etc.) in a suitable solvent (e.g. DCM, EtOH, THF, water, DMSO, dioxane etc.) at cryogenic or elevated temperature (e.g., ranging from about 0-60° C.).

The resulting PMB-amine is combined with an acid (e.g. trifluoroacetic acid, hydrochloric acid etc.) at ambient temperature (e.g., ranging from about 15-25° C.). The resulting diamine reacts with a range of different annulation reagents to provide substituted bicycloheteroaryl compounds.

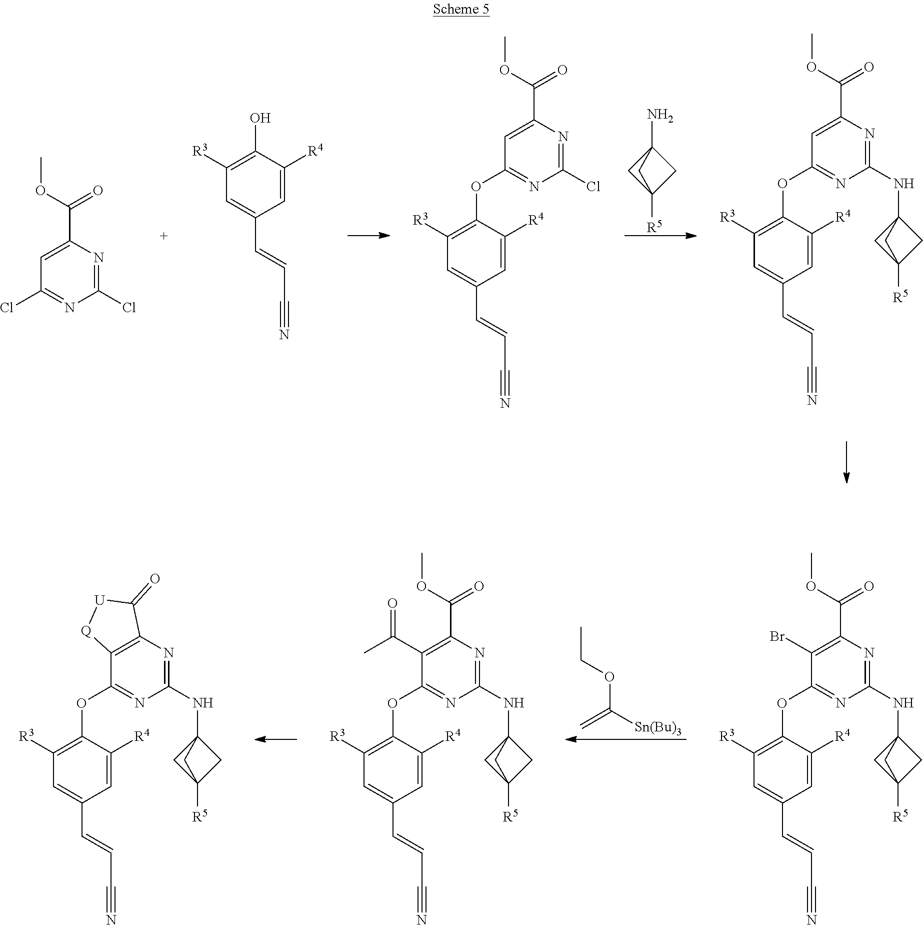

Scheme 5 shows a general synthesis of the compounds of the embodiments. The methodology is compatible with a wide variety of functionalities.

In Scheme 5, a suitably substituted heteroaryl chloride (or the corresponding bromo- or fluoro-compound) is combined with a nucleophile (e.g. aryl amine, aryl alcohol, alkyl amine etc.) in a suitable solvent system (e.g. NMP, DMF, DMAc, DMSO, acetonitrile, EtOH, THF etc.) in the presence of a base (e.g. Cs₂CO₃, K₂CO₃, triethylamine, DIPEA, NaH etc.) at ambient or elevated temperature (e.g., ranging from about 20-120° C.).

The resulting heteroaryl compound is combined with an electrophilic halogen source (N-bromosuccinimide, bromine etc) in a suitable solvent system (NMP, DMF, DMAc, DMSO, acetonitrile, EtOH, THF etc) at ambient temperature.

The resulting bromo heteroaryl compound (or the corresponding chloro- or iodo-compound) is combined with an organotin reactant (e.g. tributyl(1-ethoxyvinyl)stannane) in a suitable solvent system (e.g. DMF, DMAc, NMP, DMSO, THF, toluene etc.) in the presence of a palladium catalyst (e.g. tetrakis(triphenylphosphine)palladium(0), Pd₂(dba)₃, Pd(dppf)Cl₂ etc.) at elevated temperature (e.g., ranging from about 90-150° C.), which can be performed in microwave reactor or with conventional heating. The reaction product is combined with an acid (e.g. hydrochloric acid, phosphoric acid, acetic acid, trifluoroacetic acid etc.) at ambient temperature (e.g., ranging from about 15-25° C.).

The resulting heteroaryl keto-ester reacts with a range of different annulation reagents (e.g. alkyl amines, aryl amines, heteroaryl amines etc.) to provide substituted bicycloheteroaryl compounds.

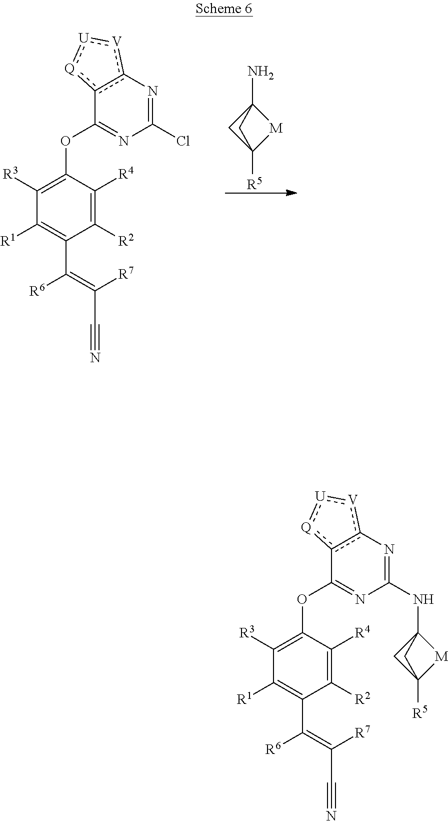

Scheme 6 shows a general synthesis of the compounds of the embodiments. The methodology is compatible with a wide variety of functionalities.

In Scheme 6, a suitably substituted heteroaryl chloride (or the corresponding bromo compound) is combined with a amine nucleophile (e.g. alkyl amine etc.) in a suitable solvent system (e.g. dioxane, THF, toluene, NMP, DMAc etc.) in the presence of a palladium catalyst (e.g. rac BINAP Pd G3, XPhos Pd G3, SPhos Pd G4, BrettPhos Pd G4, tBuXPhos Pd G3, RuPhos Pd G4 etc. and using either catalytic or stoichiometric quantities) and base (e.g. Cs₂CO₃, K₂CO₃, K₃PO₄ etc.) at elevated temperature (e.g., ranging from about 40-120° C.), which can be performed in microwave reactor or with conventional heating.

In certain instances, the above processes further involve the step of forming a salt of a compound of the present disclosure. Embodiments are directed to the other processes described herein; and to the product prepared by any of the processes described herein.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 5th edition, New York: Oxford University Press. 2009; Smith, March's Advanced Organic Chemistry: Reactions, Mechanisms, and Structure, 7th edition, Wiley-Interscience, 2013.

### List of Abbreviations and Acronyms

Abbreviation—Meaning
Ac—Acetyl
B₂pin₂—4,4,4′,4′,5,5,5′,5′-Octamethyl-2,2′-bi(1,3,2-dioxaborolane)
BAST-Bis(2-methoxyethyl)aminosulfur Trifluoride
BINAP Pd G3 [2′—(amino-κN)[1,1′-biphenyl]-2-yl-κC][[2′-(diphenylphosphino)[1,1′-binaphthalen]-2-yl]diphenylphosphine-κP](methanesulfonato-κO)-palladium
bs—Broad singlet
° C.—Degree Celsius
d—Doublet
DCM—Dichloromethane
dd—Doublet of doublet
DIPEA—N,N-Diisopropylethylamine
DMF—N,N-Dimethylformamide
DMSO—Dimethylsulfoxide
dppf 1,1′—Bis(diphenylphosphino)ferrocene
dtbpf 1,1′—Bis(di-tert-butylphosphino)ferrocene
EC50—Half maximal effective concentration
Equiv/eq—Equivalents
Et—Ethyl
EtOH—Ethanol
g—Grams
HPLC—High-performance liquid chromatography
hrs/h—Hours
Hz—Hertz
J—Coupling constant
LCMS—Liquid chromatography-mass spectrometry
LiHMDS—Lithium bis(trimethylsilyl)amide
M—Molar
m—Multiplet
m/z—mass-to-charge ratio
M+—Mass peak
Me—Methyl
mg—Milligram
MHz—Megahertz
min—Minute
mL—Milliliter
mM—Millimolar
mm—Millimeter
mmol—Millimole
mol—Mole
MS—mass spectrometry
MW—Microwave
nM—Nanomolar
NMP—N-Methyl-2-pyrrolidone
NMR—Nuclear magnetic resonance
P(oTol)3—Tri(o-tolyl)phosphine
P(t-Bu)3—Tri-tert-butylphosphine
Pd2(dba)3—Tris(dibenzylideneacetone)palladium(0)
q—Quartet
quant—Quantitative
Rf—Retention factor
RT/rt/r.t.—Room temperature
s—Singlet
sat.—Saturated
SPhos—Dicyclohexyl(2′,6′-dimethoxy-[1,1′-biphenyl]-2-yl)phosphine
t—Triplet
TFA—Trifluoroacetic acid
TMS—Trimethylsilyl
Tr/tr—Retention time
UV—Ultraviolet
wt.—Weight
Xantphos—(9,9-Dimethyl-9H-xanthene-4,5-diyl)bis(diphenylphosphine)
δ—Chemical shift
μL—Microliter
μM—Micromolar
μmol—Micromole

The following examples are merely illustrative, and do not limit this disclosure in any way.

### Example 1

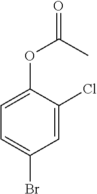

4-bromo-2-chlorophenyl acetate (Intermediate 1a): To a solution of 4-bromo-2-chlorophenol (78.0 g, 376.0 mmol) in DCM (1600 mL) was added Et₃N (76.1 g, 752.0 mmol). The mixture was cooled to 0° C., then acetyl chloride (44.3 g, 564.0 mmol) was added dropwise. The mixture was stirred at room temperature for 1 h. 1N HCl (550 mL) was added and the mixture was extracted with DCM (500 mL). The organic layer was washed with brine (800 mL×2) and dried over Na₂SO₄ and concentrated to give a crude product which was used to next step directly. ¹H NMR (400 MHz, CDCl₃) δ 7.60 (d, J=2.0 Hz, 1H), 7.41-7.39 (m, 1H), 7.03 (d, J=8.8 Hz, 1H), 2.40 (s, 3H).

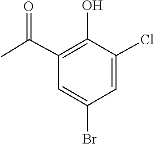

1-(5-bromo-3-chloro-2-hydroxyphenyl)ethan-1-one(Intermediate 1b): A mixture of compound Intermediate 1a (104.0 g, crude) and AlCl₃ (83.4 g, 625 mmol) was stirred at 140° C. for 2 h. The mixture was cooled to 60-70° C. and then poured into ice water (500 mL). The mixture was extracted with EtOAc (300 mL×3). The combined organic layer was washed with brine (300 mL×3) and dried over Na₂SO₄ and concentrated to give crude material which was purified by silica gel flash column chromatography (Petroleum ether/EtOAc=5: 1) to afford Intermediate 1b. MS (m/z) 246.8, 248.8 [M−H]⁻. ¹H NMR (400 MHz, CDCl₃) δ 12.74 (s, 1H), 7.78 (d, J=2.0 Hz, 1H), 7.70 (d, J=2.4 Hz, 1H), 2.67 (s, 3H).

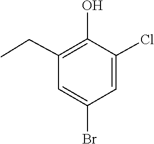

4-bromo-2-chloro-6-ethylphenol(Intermediate 1c): Sodium hydroxide (16.1 g, 402.5 mmol) and hydrazine monohydrate (24.1 g, 481.4 mmol) were added to a solution of Intermediate 1b (40.0 g, 160.3 mmol) dissolved in triethylene glycol (200 mL). The reaction mixture was heated to 160° C. overnight. The mixture was cooled to room temperature was adjusted to pH 5 with 1 N HCl then extracted with EtOAc (200 ml×2). The organic layer was washed with saturated NaHCO₃, brine, dried over Na₂SO₄ and concentrated in vacuo to yield a crude residue which was subjected to silica gel flash chromatography to afford the title compound. MS (m/z) 233.0, 234.0 [M−H]⁻. ¹H NMR (400 MHz, CDCl₃) δ 7.31 (d, J=2.0 Hz, 1H), 7.49 (d, J=2.0 Hz, 1H), 5.55 (s, 1H), 2.69 (q, J=7.2 Hz, 2H), 1.23 (t, J=5.2 Hz, 3H).

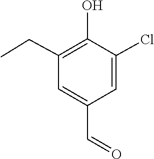

3-chloro-5-ethyl-4-hydroxybenzaldehyde (Intermediate 1d): To a mixture of compound Intermediate 1c (22.0 g, 93.4 mmol) in THE (325 mL) was added dropwise n-BuLi (2.5M in n-hexane, 82 mL, 205.5 mmol) at −78° C. under N₂ atmosphere. Then the mixture was stirred at −70° C. for 2 h. DMF (28.0 g, 382.9 mmol) was then added dropwise at −78° C. The mixture was then stirred at room temperature overnight. The mixture was quenched with 2 N HCl and adjusted to pH 3-4. The mixture was extracted with EtOAc (300 mL×3) and the organic phase was washed with brine (300 mL×3), dried over Na₂SO₄ and concentrated in vacuo. The crude product was subjected to silica gel flash column chromatography (Petroleum ether/EtOAc=15: 1 to 10: 1) to afford the title compound. MS (m/z) 183.0 [M−H]⁻. ¹H NMR (400 MHz, CDCl₃) δ 9.82 (s, 1H), 7.75 (d, J=2.0 Hz, 1H), 7.62 (s, 1H), 6.13 (s, 1H), 2.78 (q, J=7.6 Hz, 2H), 1.27 (t, J=7.6 Hz, 3H).

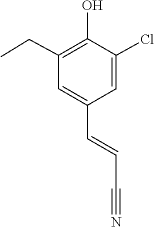

(E)-3-(3-chloro-5-ethyl-4-hydroxyphenyl)acrylonitrile (Intermediate 1e): To a mixture of diethyl (cyanomethyl)phosphonate (13.7 g, 77.5 mmol) in THE (220 mL) was added NaH (6.0 g, 149.0 mmol) at 0° C. The mixture was stirred at room temperature for 0.5 h then was cooled to 0° C. and a mixture of Intermediate 1d (11.0 g, 59.6 mmol) in THE (110 mL) was added dropwise at 0° C. The mixture was stirred at 0° C. for 5 h. The mixture was quenched with water (300 mL) and adjusted to pH 2 with 1N HCl. The solution was extracted with EtOAc (400 mL×3) and the isolated organic phase was washed with brine (400 mL×3), dried over Na₂SO₄ and concentrated en vacuo. The crude material was subjected to silica gel flash column chromatography (Petroleum ether/EtOAc=5: 1) to afford crude product, which was triturated with heptane to give the title compound. MS (m/z) 206.0 [M−H]⁻. ¹H NMR (400 MHz, DMSO-d₆) δ ppm 9.79 (brs, 1H), 7.58 (d, J=2.0 Hz, 1H), 7.50 (d, J=16.8 Hz, 1H), 7.40 (d, J=2.0 Hz, 1H), 6.33 (d, J=16.4 Hz, 1H), 2.64 (q, J=7.2 Hz, 2H), 1.14 (t, J=7.6 Hz, 3H).

### Preparation of Compound 1 (E)-3-((4-(4-(2-cyanovinyl)-2,6-dimethyl-3-(trifluoromethyl)phenoxy)-2-methyl-2H-pyrazolo[3,4-d]pyrimidin-6-yl)amino)bicyclo[1.1.1]pentane-1-carbonitrile

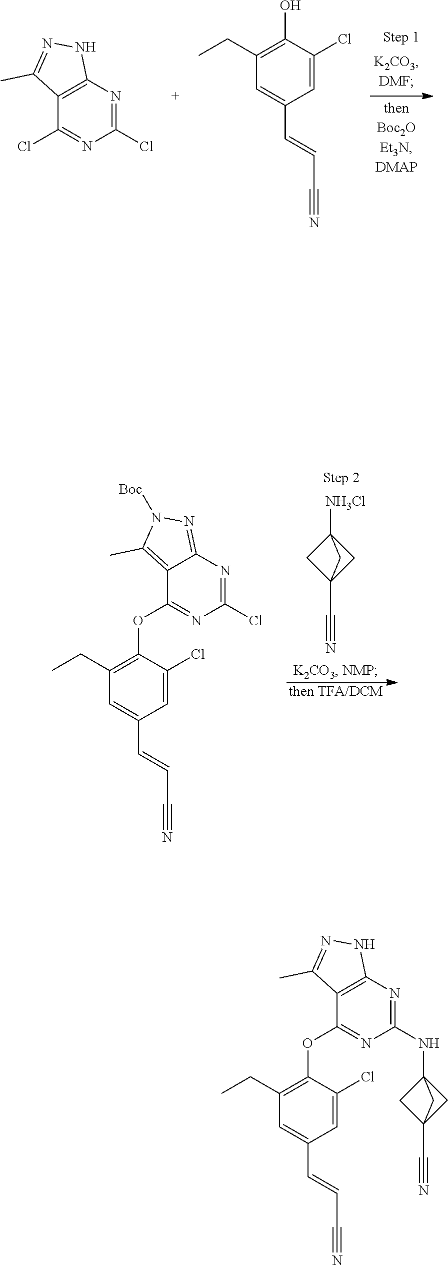

Step 1, preparation of Intermediate 1f tert-butyl (E)-6-chloro-4-(2-chloro-4-(2-cyanovinyl)-6-ethylphenoxy)-3-methyl-2H-pyrazolo[3,4-d]pyrimidine-2-carboxylate (Intermediate 1f). (E)-3-(3-chloro-5-ethyl-4-hydroxyphenyl)acrylonitrile (1.23 mmol, 256 mg), 4,6-dichloro-3-methyl-1H-pyrazolo[3,4-d]pyrimidine (1.23 mmol, 250 mg), and potassium carbonate (3.69 mmol, 511 mg) were suspended in DMF (5.0 mL). The mixture was stirred at room temperature for one hour. To the mixture was then added Boc₂O (1.85 mmol, 403 mg), triethylamine (6.16 mmol, 0.86 mL), and DMAP (0.123 mmol, 15.0 mg). The mixture was stirred at room temperature for 1.5 hours at which point water (40 mL) was added to the mixture. The mixture was vigorously stirred and then subsequently filtered to yield a solid. This solid was purified via silica gel chromatography (0-100% EtOAc in hexanes) to afford the title compound (180 mg, 31% yield). MS (m/z) 473.46 [M+H]+. ¹H NMR (400 MHz, DMSO-d₆) δ 7.89 (s, 1H), 7.73 (s, 1H), 7.69 (d, J=16.8 Hz, 1H), 6.64 (d, J=16.7, 1H), 2.67 (s, 3H), 2.57 (m, 2H), 1.63 (s, 9H), 1.22-1.11 (m, 3H).

Step 2, preparation of (E)-3-((4-(2-chloro-4-(2-cyanovinyl)-6-ethylphenoxy)-3-methyl-1H-pyrazolo[3,4-d]pyrimidin-6-yl)amino)bicyclo[1.1.1]pentane-1-carbonitrile: Intermediate if (0.105 mmol, 50.0 mg), 3-aminobicyclo[1.1.1]pentane-1-carbonitrile; hydrochloride (0.211 mmol, 30.5 mg), and potassium carbonate (0.527 mmol, 72.8 mg) were suspended in NMP (0.3 mL). The mixture was heated to 50° C. and stirred at this temperature overnight. The mixture was then filtered using 1 mL DCM to wash the filtered solids. To the combined filtrate was added 1 mL of TFA and the mixture was stirred at room temperature for two hours. The mixture was concentrated in vacuo and purified via preparative HPLC (0-100% MeCN in water, 0.1% TFA) then lyophilized to afford the title compound. MS (m/z) 446.25 [M+H]+. ¹H NMR (400 MHz, Chloroform-d) δ 7.56 (d, J=2.0 Hz, 1H), 7.44-7.39 (m, 2H), 5.99 (d, J=16.7 Hz, 1H), 2.75 (s, 3H), 2.70-2.57 (m, 2H), 2.23 (br s, 6H), 1.32-1.19 (m, 3H).

### Example 2

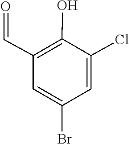 [SMILES: O=Cc1cc(Br)cc(Cl)c1O]

5-bromo-3-chloro-2-hydroxybenzaldehyde (Intermediate 2a): 4-bromo-2-chlorophenol (50.0 g, 241.0 mmol) was dissolved in TFA (500 mL) at 0° C., then hexamethylenetetramine (67.6 g, 482.0 mmol) was added at room temperature. The mixture was heated to 90° C. and stirred for 12 h. The mixture was cooled to room temperature and poured into a mixture of water (500 mL) and 50% aqueous H₂SO₄ (250 mL). The mixture was filtered and the filter cake was dried under vacuum at 45° C. for 16 h to give the title compound which was used for next step directly. MS (m/z) 232.8, 234.8 [M−H]⁻. ¹H NMR (300 MHz, DMSO-d₆) δ 11.21 (s, 1H), 10.11 (s, 1H), 7.98 (d, J=2.4 Hz, 1H), 7.83 (d, J=2.7 Hz, 1H).

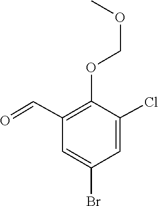

5-bromo-3-chloro-2-(methoxymethoxy)benzaldehyde (Intermediate 2b): To a mixture of Intermediate 2a (45.0 g, 191.1 mmol) in DCM (1350 mL) was added MOMBr (59.7 g, 477.8 mmol) and DIEA (98.8 g, 764.4 mmol) at room temperature under N₂ atmosphere. Then the mixture was stirred at room temperature for 6 h. The mixture was poured into saturated aqueous NH₄Cl (900 mL). After separation, the aqueous layer was extracted with DCM (400 mL×3). The combined organic layers were washed with brine (900 mL), dried over Na₂SO₄ and concentrated to give a crude product, which was purified by silica gel flash column chromatography (Petroleum ether/EtOAc=30: 1) to give the title compound. ¹H NMR (400 MHz, DMSO-d₆) δ 10.13 (s, 1H), 8.15 (d, J=2.4 Hz, 1H), 7.82 (d, J=2.4 Hz, 1H), 5.21 (s, 2H), 3.53 (s, 3H).

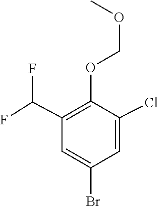

5-bromo-1-chloro-3-(difluoromethyl)-2-(methoxymethoxy)benzene (Intermediate 2c): To a mixture of Intermediate 2b (32.0 g, 114.5 mmo) in DCM (450 mL) was added BAST (63.0 g, 284.8 mmol) and EtOH (1.5 g, 32.6 mmol) at room temperature under N₂ atmosphere. The mixture was stirred at room temperature overnight. The mixture was poured into saturated aqueous NaHCO₃ (500 mL). After separation and the aqueous layer was extracted with DCM (300 mL×2). The combined organic phases were washed with brine (300 mL×3) and dried over Na₂SO₄ and concentrated to give a crude product, which was purified by silica gel flash column chromatography (Petroleum ether/EtOAc=30: 1) to give the title compound. ¹H NMR (300 MHz, DMSO-d₆) δ 8.00 (t, J=1.2 Hz, 1H), 7.72 (d, J=2.4 Hz, 1H), 7.10 (t, J=54.3 Hz, 1H), 5.13 (s, 2H), 3.53 (s, 3H).

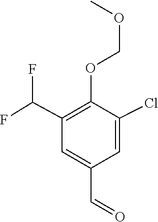 [SMILES: COCOc1c(Cl)cc(C=O)cc1C(F)F]

3-chloro-5-(difluoromethyl)-4-(methoxymethoxy)benzaldehyde (Intermediate 2d): To a mixture of compound Intermediate 2c (21.0 g, 69.6 mmol) in THE (275 mL) was added dropwise n-BuLi (2.5M in n-hexane, 61 mL, 153.1 mmol) at −78° C. under N₂ atmosphere. Then the mixture was stirred at −78° C. for 2 h. To the mixture was added dropwise DMF (20.9 g, 285.4 mmol) at −78° C. Then the mixture was stirred at −78° C. for 2 h. The mixture was quenched with 2 N HCl and adjusted pH to 3-4. The solution was extracted with EtOAc (200 mL×3). The combined organic phases were washed with brine (200 mL×3), dried over Na₂SO₄ and concentrated to give a crude product (21.0 g, crude) which was used for next step without further purification.

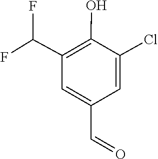 [SMILES: O=Cc1cc(Cl)c(O)c(C(F)F)c1]

3-chloro-5-(difluoromethyl)-4-hydroxybenzaldehyde(Intermediate 2e): To a mixture of Intermediate 2d (52.0 g, 207.5 mmol) in MeOH (520 mL) was added HCl (312 mL) at room temperature and the mixture was stirred at room temperature for 2 h. The mixture was poured into ice water (800 mL) and was extracted with MTBE (400 mL×3). The combined organic layers were washed with brine (300 mL×3), dried over Na₂SO₄ and concentrated to give a crude product, which was purified by silica gel flash column chromatography (Petroleum ether/EtOAc=10: 1) to give the title compound. MS (m/z) 205.0 [M−H]⁻. ¹H NMR (300 MHz, DMSO-d₆) δ 9.87 (s, 1H), 8.07 (t, J=0.9 Hz, 1H), 7.97 (t, J=0.6 Hz, 1H), 7.19 (t, J=54.6 Hz, 1H).

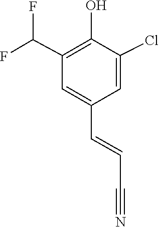 [SMILES: N#C/C=C/c1cc(Cl)c(O)c(C(F)F)c1]

(E)-3-(3-chloro-5-(difluoromethyl)-4-hydroxyphenyl)acrylonitrile (Intermediate 2f): To a mixture of diethyl (cyanomethyl)phosphonate (22.3 g, 125.8 mmol, 2.0 eq) in THE (250 mL) was added NaH (10.1 g, 251.6 mmol) in portions at 0° C. Then the mixture was stirred at room temperature for 0.5 h. The mixture was cooled to 0° C. and a mixture of Intermediate 2e (13.0 g, 62.9 mmol, 1.0 eq) in THF (110 mL) was added dropwise at 0° C. The mixture was stirred at 0° C. for 5 h. The mixture was quenched with water (300 mL) and adjusted pH to 2 with 1N HCl. The solution was extracted with EtOAc (400 mL×3). The combined organic phases were washed with brine (400 mL×3), dried over Na₂SO₄ and concentrated. The crude product was purified by silica gel column chromatography (Petroleum ether/EtOAc=5: 1) to give crude product, which was triturated with heptane (50 mL) and then further purified by SFC separation D to give the title compound as a light yellow solid. MS (m/z) 228.0 [M−H]⁻. ¹H NMR (300 MHz, DMSO-d₆): δ ppm 11.09 (brs, 1H), 7.95 (s, 1H), 7.74 (s, 1H), 7.67 (d, J=14.4 Hz, 1H), 7.14 (t, J=54.9 Hz, 1H), 6.49 (d, J=16.5 Hz, 1H).

The following compounds were prepared using the two-step procedure described for the synthesis of Compound 1 (E)-3-((4-(2-chloro-4-(2-cyanovinyl)-6-ethylphenoxy)-3-methyl-1H-pyrazolo[3,4-d]pyrimidin-6-yl)amino)bicyclo[1.1.1]pentane-1-carbonitrile, with the following modifications:

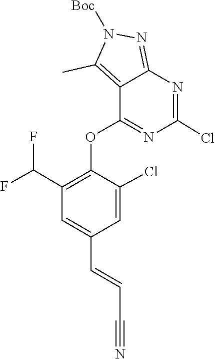 [SMILES: Cc1c2c(Oc3c(Cl)cc(/C=C/C#N)cc3C(F)F)nc(Cl)nc2nn1C(=O)OC(C)(C)C]

tert-but(E)(E)-6-chloro-4-(2-chloro-4-(2-cyanovinyl)-6-(difluoromethyl)phenoxy)-3-methyl-2H-pyrazolo[3,4-d]pyrimidine-2-carboxylate (Intermediate 2g): (E)-3-(3-chloro-5-(difluoromethyl)-4-hydroxyphenyl)acrylonitrile was used in place of (E)-3-(4-hydroxy-3,5-dimethyl-2-(trifluoromethyl)phenyl)acrylonitrile to afford the title compound. ¹H NMR (400 MHz, DMSO-d6) δ 8.27 (s, 1H), 8.05 (s, 1H), 7.78 (d, J=16.7 Hz, 1H), 7.21 (t, J=53.6 Hz, 1H), 6.75 (d, J=16.8 Hz, 1H), 2.66 (s, 3H), 1.63 (s, 9H).

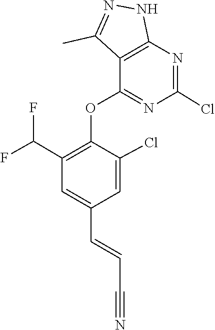 [SMILES: Cc1n[nH]c2nc(Cl)nc(Oc3c(Cl)cc(/C=C/C#N)cc3C(F)F)c12]

(E)-3-((4-(2-chloro-4-(2-cyanovinyl)-6-(difluoromethyl)phenoxy)-3-methyl-11H-pyrazolo[3,4-d]pyrimidin-6-yl)amino)bicyclo[1.1.1]pentane-1-carbonitrile Compound 2: Intermediate 2g was used in place of Intermediate if and the product was purified by preparative HPLC (0-100% MeCN in water, 0.1% TFA) to afford the title compound. MS (m/z) 468.29, 470.18 [M+H]+. ¹H NMR (400 MHz, Acetonitrile-d3) δ 7.97 (s, 1H), 7.87 (s, 1H), 7.56 (d, J=16.7 Hz, 1H), 6.89 (t, J=54.1 Hz, 1H), 6.26 (d, J=16.7 Hz, 1H), 2.58 (s, 3H), 2.15 (s, 6H). ¹⁹F NMR (376 MHz, CD3CN) δ −77.32, −116.22.

### Example 3

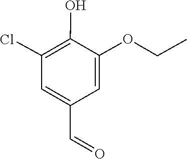

3-chloro-5-ethoxy-4-hydroxybenzaldehyde (Intermediate 3a): To a solution of 3-ethoxy-4-hydroxybenzaldehyde (20.0 g, 120.4 mmol) in CCl₄ (200 mL) was added DMSO (1.9 g, 24.1 mmol). To the mixture was added NCS (80.4 g, 602.0 mmol) at 0° C. and the mixture was allowed to slowly warm to room temperature and stir overnight. A saturated aqueous solution of Na₂S₂O₃ (100 mL) was added to the mixture and the mixture was extracted with DCM (200 mL×3). The combined organic phases were washed with brine (200 mL), dried over Na₂SO₄, filtered and concentrated in vacuo. The resulting residue was subjected to silica gel flash column chromatography (Petroleum ether/EtOAc=10 to 1) and then triturated with MTBE (30 mL) to give Intermediate 3a. MS (m/z) 199.0 [M−H]⁻. ¹H NMR (300 MHz, DMSO-d₆) δ 10.47 (s, 1H), 9.77 (s, 1H), 7.58 (s, 1H), 7.38 (s, 1H), 4.17 (q, J=7.0 Hz, 2H), 1.38 (t, J=7.0 Hz, 3H).

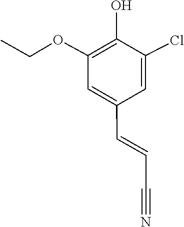

(E)-3-(3-chloro-5-ethoxy-4-hydroxyphenyl)acrylonitrile (Intermediate 3b): To a solution of diethyl (cyanomethyl)phosphonate (7.6 g, 42.8 mmol) in THE (40 mL) was added NaH (3.3 g, 82.2 mmol) at 0° C. under N₂ atmosphere. The mixture was stirred at room temperature for 0.5 h at which point Intermediate 3a(6.6 g, 32.9 mmol) in THE (30 mL) was added dropwise at 0° C. The mixture was stirred at room temperature overnight. The mixture was quenched with water (50 mL), adjusted pH to 2 with 1N HCl (40 mL). The solution was extracted with EtOAc (50 mL×3). The organic phase was washed with brine (100 mL), dried over Na₂SO₄ and concentrated in vacuo. The resulting residue was subjected to silica gel flash column chromatography (Petroleum ether/EtOAc=10 to 1) and triturated with MTBE (20 mL) to yield the title compound. MS (m/z) 222.0 [M−H]⁻. ¹H NMR (300 MHz, DMSO-d₆) δ 9.92 (s, 1H), 7.48 (d, J=16.6 Hz, 1H), 7.29 (dd, J=9.3, 1.9 Hz, 2H), 6.37 (d, J=16.6 Hz, 1H), 4.12 (q, J=7.0 Hz, 2H), 1.36 (t, J=7.0 Hz, 3H).

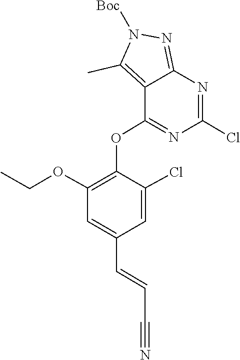 [SMILES: CCOc1cc(/C=C/C#N)cc(Cl)c1Oc1nc(Cl)nc2nn(C(=O)OC(C)(C)C)c(C)c12]

tert-butyl (E)-6-chloro-4-(2-chloro-4-(2-cyanovinyl)-6-ethoxyphenoxy)-3-methyl-2H-pyrazolo[3,4-d]pyrimidine-2-carboxylate (Intermediate 3c): (E)-3-(3-chloro-5-ethoxy-4-hydroxyphenyl)acrylonitrile was used in place of (E)-3-(4-hydroxy-3,5-dimethyl-2-(trifluoromethyl)phenyl)acrylonitrile to afford the title compound (145 mg, 24% yield). MS (m/z) 492.18 [M+H]+.

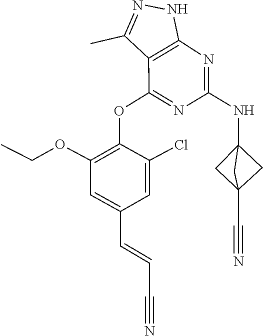 [SMILES: CCOc1cc(/C=C/C#N)cc(Cl)c1Oc1nc(NC23CC(C#N)(C2)C3)nc2[nH]nc(C)c12]

(E)-3-((4-(2-chloro-4-(2-cyanovinyl)-6-ethoxyphenoxy)-3-methyl-11H-pyrazolo[3,4-d]pyrimidin-6-yl)amino)bicyclo[1.1.1]pentane-1-carbonitrile Compound 3: Intermediate 3a was used in place of Intermediate 1f and the product was purified by preparative HPLC (0-100% MeCN in water, 0.1% TFA) to afford the title compound. MS (m/z) 462.25, 464.24 [M+H]+. ¹H NMR (400 MHz, Acetonitrile-d3) δ 7.48 (d, J=16.7 Hz, 1H), 7.38 (s, 1H), 7.26 (s, 1H), 6.23-6.15 (m, 1H), 4.21-4.03 (m, 2H), 2.58 (s, 3H), 2.16 (s, 6H), 1.19 (t, J=7.0 Hz, 3H).

### Example 4

### (E)-3-((4-(4-(2-cyanovinyl)-2,6-dimethyl-3-(trifluoromethyl)phenoxy)-2-methyl-2H-pyrazolo[3,4-d]pyrimidin-6-yl)amino)bicyclo[1.1.1]pentane-1-carbonitrile-Compound 4

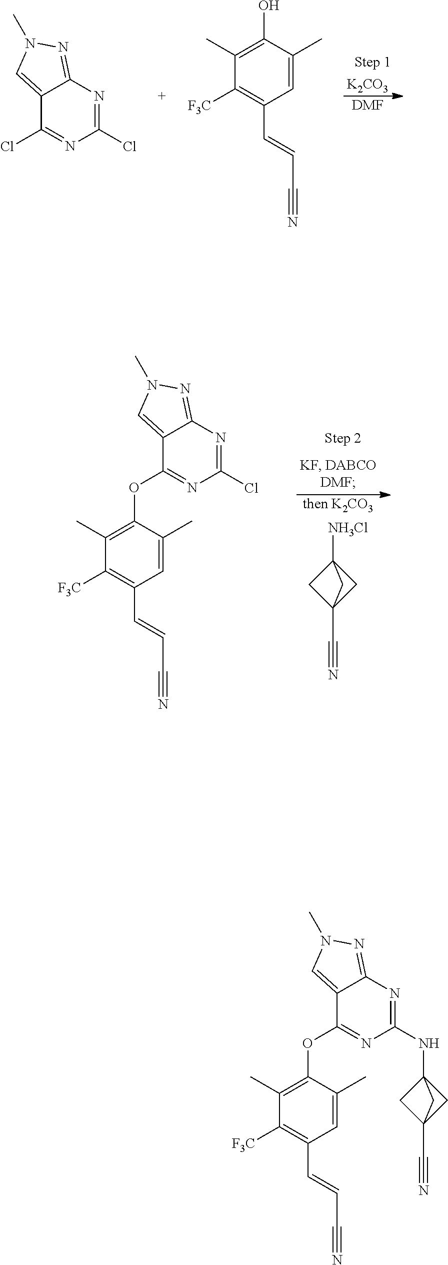

Step 1, Intermediate 4a(E)-3-(4-((6-chloro-2-methyl-2H-pyrazolo[3,4-d]pyrimidin-4-yl)oxy)-3,5-dimethyl-2-(trifluoromethyl)phenyl)acrylonitrile (c): (E)-3-(4-hydroxy-3,5-dimethyl-2-(trifluoromethyl)phenyl)acrylonitrile (1.63 mmol, 392 mg), 4,6-dichloro-2-methyl-pyrazolo[3,4-d]pyrimidine (1.48 mmol, 300 mg), and potassium carbonate (2.22 mmol, 306 mg) were suspended in DMF (6.0 mL). The mixture was stirred at room temperature for 2 h at which point water (20 mL) was added to the mixture. The mixture was vigorously stirred and then subsequently filtered to yield a damp solid. This solid was further dried under vacuum to afford the title compound. MS (m/z) 408.13 [M+H]+.

Step 2, preparation of (E)-3-((4-(4-(2-cyanovinyl)-2,6-dimethyl-3-(trifluoromethyl)phenoxy)-2-methyl-2H-pyrazolo[3,4-d]pyrimidin-6-yl)amino)bicyclo[1.1.1]pentane-1-carbonitrile Compound 4. Intermediate 4a (0.123 mmol, 50.0 mg), potassium fluoride (0.61 mmol, 36 mg), and 1,4-diazabicyclo[2.2.2]octane (0.061, mmol, 6.9 mg) were suspended in DMF (0.5 mL). The mixture was stirred at 60° C. for two hours and then cooled to room temperature. To the mixture was added 3-aminobicyclo[1.1.1]pentane-1-carbonitrile; hydrochloride (0.31 mmol, 44 mg) and potassium carbonate (0.61 mmol, 85 mg). The mixture stirred at 50° C. overnight, at which point the mixture was then cooled to room temperature and passed through a filter. The filtrate was purified by preparative HPLC (0-100% MeCN in water) then lyophilized to afford the title compound (4.2 mg, 7.1% yield). MS (m/z) 480.19 [M+H]+. ¹H NMR (400 MHz, DMSO-d6) δ 8.57 (s, 1H), 7.90 (m, 2H), 7.65 (s, 1H), 6.30 (d, J=16.3 Hz, 1H), 4.04 (s, 3H), 2.21 (m, 3H), 2.15 (s, 3H), 2.00 (br s, 6H). ¹⁹F NMR (376 MHz, DMSO-d6) δ −51.99.

### Example 5

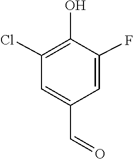 [SMILES: O=Cc1cc(F)c(O)c(Cl)c1]

3-chloro-5-fluoro-4-hydroxybenzaldehyde (Intermediate 5a): To a mixture of 2-chloro-6-fluorophenol (20.0 g, 136.5 mmol) in TFA (40 mL) was added dropwise a solution of hexamethylenetetramine (38.3 g, 273.0 mmol) in TFA (100 mL) at 60° C. Then the mixture was stirred at 70° C. for 12 h. The mixture was cooled to room temperature and poured into water (300 mL). The precipitated solid was collected by filtration, washed with water (100 mL) and dried to give a residue which was triturated with 10:1 Petroleum ether: EtOAc (150 mL) and filtered to give the title compound. MS (m/z) 173.0 [M−H]⁻. ¹H NMR (300 MHz, DMSO-d₆) δ ppm 11.79 (br s, 1H), 9.80 (d, J=1.8 Hz, 1H), 7.83 (s, 1H), 7.69 (dd, J=1.8 Hz, 10.4 Hz, 1H).

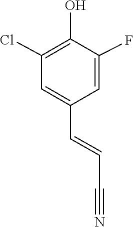 [SMILES: N#C/C=C/c1cc(F)c(O)c(Cl)c1]

(E)-3-(3-chloro-5-fluoro-4-hydroxyphenyl)acrylonitrile (Intermediate 5b): To a mixture of diethyl (cyanomethyl)phosphonate (26.4 g, 148.9 mmol) in THE (200 mL) was added NaH (11.5 g, 286.4 mmol) at 0° C. Then the mixture was stirred at room temperature for 30 min. The mixture was cooled to 0° C. and a mixture of Intermediate 5a (20.0 g, 114.6 mmol) in THE (100 mL) was added dropwise at 0° C. The mixture was stirred at 0° C. for 5 h. The mixture was quenched with water (250 mL) and the pH was acidified to 2 with 1N HCl. The solution was extracted with EtOAc (300 mL×3). The organic phase was washed with brine (250 mL×3), dried over Na₂SO₄ and concentrated in vacuo. The resulting residue was subjected to silica gel flash column chromatography (Petroleum ether: EtOAc=5: 1-3: 1) and recrystallized with EtOAc (80 mL) to afford the title compound. MS (m/z) 196.0 [M−H]⁻. ¹H NMR (400 MHz, DMSO-d₆) δ ppm 11.19 (br s, 1H), 7.61-7.55 (m, 2H), 7.50 (d, J=16.6 Hz, 1H), 6.42 (d, J=16.6 Hz, 1H).

The following compounds were prepared using the two-step procedure described for the synthesis of (E)-3-((4-(4-(2-cyanovinyl)-2,6-dimethyl-3-(trifluoromethyl)phenoxy)-2-methyl-2H-pyrazolo[3,4-d]pyrimidin-6-yl)amino)bicyclo[1.1.1]pentane-1-carbonitrile, with the following modifications:

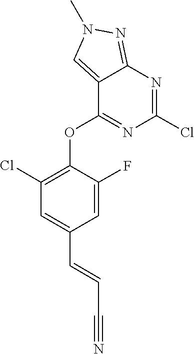 [SMILES: Cn1cc2c(Oc3c(F)cc(/C=C/C#N)cc3Cl)nc(Cl)nc2n1]

(E)-3-(3-chloro-4-((6-chloro-2-methyl-2H-pyrazolo[3,4-d]pyrimidin-4-yl)oxy)-5-fluorophenyl)acrylonitrile (Intermediate 5c): (E)-3-(3-chloro-5-fluoro-4-hydroxyphenyl)acrylonitrile (1.63 mmol, 321 mg) was used in place of (E)-3-(4-hydroxy-3,5-dimethyl-2-(trifluoromethyl)phenyl)acrylonitrile to afford the title compound. MS (m/z) 365.91 [M+H]+.

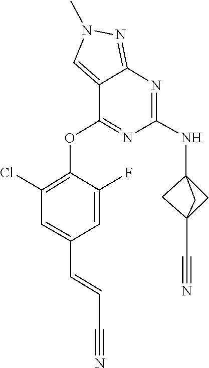 [SMILES: Cn1cc2c(Oc3c(F)cc(/C=C/C#N)cc3Cl)nc(NC34CC(C#N)(C3)C4)nc2n1]

(E)-3-((4-(2-chloro-4-(2-cyanovinyl)-6-fluorophenoxy)-2-methyl-2H-pyrazolo[3,4-d]pyrimidin-6-yl)amino)bicyclo[1.1.1]pentane-1-carbonitrile—Compound 5: Intermediate 5c (0.137 mmol, 50.0 mg) was used in place of Intermediate 4a Intermediate 4a and the product was purified by preparative HIPLC (0-100% MeCN in water, 0.1% TFA) to afford the title compound. MS (m/z): 436.25, 438.16 [M+H]+. ¹H NMR (400 MHz, Acetonitrile-d3) δ 8.32 (s, 1H), 7.69 (s, 1H), 7.59 (d, J=10.3 Hz, 1H), 7.52 (d, J=16.7 Hz, 1H), 6.24 (d, J=16.7 Hz, 1H), 4.10 (d, J=0.6 Hz, 3H), 2.26 (s, 6H).

### Example 6

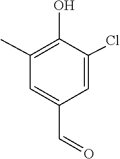

3-chloro-4-hydroxy-5-methylbenzaldehyde (Intermediate 6a): To a solution of 4-bromo-2-chloro-6-methylphenol(37.0 g, 167.1 mmol) in THE (462 mL) was added dropwise n-BuLi (2.5 M in n-hexane, 150 mL) at −70° C. under N₂ atmosphere. The mixture was stirred at −70° C. for 2 h. To the mixture was added dropwise DMF (50.1 g, 684.9 mmol). The mixture was stirred at room temperature for 12 h. 2N HCl (300 mL) was added and the mixture was stirred at 0° C. for 15 min. Saturated aqueous NaHCO₃ was added to adjust the pH to 8 and the mixture was extracted with EtOAc (300 mL×3). The combined organic layers were dried over Na₂SO₄, filtered, and concentrated in vacuo to give a residue, which was subjected to silica gel flash column chromatography (Petroleum ether/EtOAc=10: 1) to afford the title compound. MS (m/z) 169.0 [M−H]⁻. ¹H NMR (400 MHz, DMSO-d₆) δ 10.36 (s, 1H), 9.77 (s, 1H), 7.76 (s, 1H), 7.64 (s, 1H), 2.27 (s, 3H).

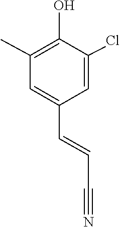

(E)-3-(3-chloro-4-hydroxy-5-methylphenyl)acrylonitrile (Intermediate 6b): To a solution of diethyl (cyanomethyl)phosphonate (13.5 g, 76.2 mmol) in THE (100 mL) was added NaH (5.9 g, 146.5 mmol) at 0° C. The mixture was stirred at room temperature for 15 min. Then to the mixture was added dropwise a solution of Intermediate 6a (10.0 g, 58.6 mmol) in THE (200 mL). The mixture was stirred at room temperature for 12 h. Water (200 mL) was added into the crude mixture and 1N HCl was added to adjust the pH to 2. The aqueous layer was extracted with EtOAc (250 mL×2). The combined organic layers were dried over Na₂SO₄, filtered, and concentrated to give a crude product, which was purified by silica gel (eluted with Petroleum ether/EtOAc=1: 1) to afford the title compound. MS (m/z) 192.0 [M−H]⁻.

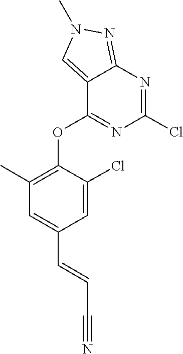 [SMILES: Cc1cc(/C=C/C#N)cc(Cl)c1Oc1nc(Cl)nc2nn(C)cc12]

(E)-3-(3-chloro-4-((6-chloro-2-methyl-2H-pyrazolo[3,4-d]pyrimidin-4-yl)oxy)-5-methylphenyl)acrylonitrile (Intermediate 6c): (E)-3-(3-chloro-4-hydroxy-5-methyl-phenyl)prop-2-enenitrile (1.48 mmol, 315 mg) was used in place of (E)-3-(4-hydroxy-3,5-dimethyl-2-(trifluoromethyl)phenyl)acrylonitrile to afford the title compound. MS (m/z) 360.15, 362.10 [M+H]+.

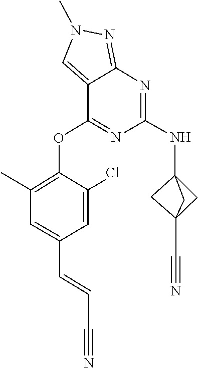 [SMILES: Cc1cc(/C=C/C#N)cc(Cl)c1Oc1nc(NC23CC(C#N)(C2)C3)nc2nn(C)cc12]

(E)-3-((4-(2-chloro-4-(2-cyanovinyl)-6-methylphenoxy)-2-methyl-2H-pyrazolo[3,4-d]pyrimidin-6-yl)amino)bicyclo[1.1.1]pentane-1-carbonitrile—Compound 6: Intermediate 6c was used in place of Intermediate 4a Intermediate 4a and the product was purified by preparative HPLC (0-100% MeCN in water, 0.1% TFA) to afford the title compound. MS (m/z) 432.21 [M+H]+. ¹H NMR (400 MHz, DMSO-d6) δ 8.55 (s, 1H), 7.88 (s, 2H), 7.71 (s, 1H), 7.66 (d, J=16.7 Hz, 1H), 6.59 (d, J=16.7 Hz, 1H), 4.04 (s, 3H), 2.16 (s, 3H), 2.15 (br s, 6H).

### Example 7

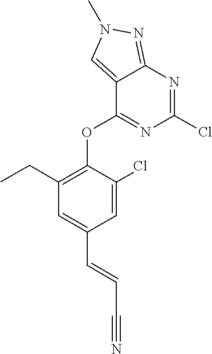 [SMILES: CCc1cc(/C=C/C#N)cc(Cl)c1Oc1nc(Cl)nc2nn(C)cc12]

(E)-3-(3-chloro-4-((6-chloro-2-methyl-2H-pyrazolo[3,4-d]pyrimidin-4-yl)oxy)-5-ethylphenyl)acrylonitrile (Intermediate 7a): (E)-3-(3-chloro-4-hydroxy-5-ethyl-phenyl)prop-2-enenitrile (1.48 mmol, 315 mg) was used in place of (E)-3-(4-hydroxy-3,5-dimethyl-2-(trifluoromethyl)phenyl)acrylonitrile to afford the title compound. MS (m/z) 374.10, 376.09 [M+H]+.

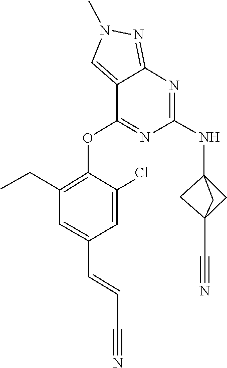 [SMILES: CCc1cc(/C=C/C#N)cc(Cl)c1Oc1nc(NC23CC(C#N)(C2)C3)nc2nn(C)cc12]

(E)-3-((4-(2-chloro-4-(2-cyanovinyl)-6-ethylphenoxy)-2-methyl-2H-pyrazolo[3,4-d]pyrimidin-6-yl)amino)bicyclo[1.1.1]pentane-1-carbonitrile—Compound 7: Intermediate 7a (0.13 mmol, 50 mg) was used in place of Intermediate 4a and the product was purified by preparative HPLC (0-100% MeCN in water, 0.1% TFA) to afford the title compound. MS (m/z) 446.25, 448.18 [M+H]+. ¹H NMR (400 MHz, Acetonitrile-d3) δ 8.36 (s, 1H), 7.72 (d, J=2.1 Hz, 1H), 7.60 (d, J=2.1 Hz, 1H), 7.54 (d, J=16.7 Hz, 1H), 6.22 (d, J=16.7 Hz, 1H), 4.10 (d, J=0.6 Hz, 3H), 2.61 (q, J=7.6 Hz, 2H), 2.17 (s, 6H), 1.20 (t, J=7.6 Hz, 3H).

### Example 8

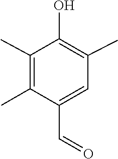

4-hydroxy-2,3,5-trimethylbenzaldehyde (Intermediate 8a): To a mixture of 2,3,6-trimethylphenol (50.0 g, 367.1 mmol) in AcOH (150 mL) was added a solution of hexamethylenetetramine (25.7 g, 183.6 mmol) in AcOH (50 mL) dropwise at 120° C. Then the mixture was stirred at 120° C. for 3 h. The mixture was then cooled to 100° C. and 20% H₂SO₄ (200 mL) was added. Then the mixture was stirred for 0.5 h. The mixture was poured into ice water (200 mL) and filtered to afford the title compound. MS (m/z) 165.2 [M+H]⁺.

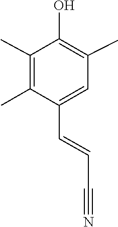

(E)-3-(4-hydroxy-2,3,5-trimethylphenyl)acrylonitrile (Intermediate 8b): To a mixture of diethyl (cyanomethyl)phosphonate (129.5 g, 730.8 mmol) in THE (300 mL) was added NaH (24.4 g, 609.0 mmol) at 0° C. Then the mixture was stirred at room temperature for 30 min. The mixture was cooled to 0° C. and a solution of Intermediate 8a (40.0 g, 243.6 mmol) in THE (500 mL) was added dropwise at 0° C. The mixture was stirred at room temperature for 5 h. The mixture was then quenched with water (400 mL), adjusted to pH 2 with 1N HCl (500 mL) and extracted with EtOAc (500 mL×3). The combined organic phases were washed with brine (250 mL×3), dried over Na₂SO₄, filtered, and concentrated in vacuo. The crude product was subjected to silica gel flash column chromatography (Petroleum ether: EtOAc=5: 1) and recrystallized with THF/n-hexane (10: 1) to afford the title compound. MS (m/z) 186.0 [M−H]⁻. ¹H NMR (400 MHz, DMSO-d₆) δ 8.73 (s, 1H), 7.80 (d, J=16.4 Hz, 1H), 7.32 (s, 1H), 6.06 (d, J=16.4 Hz, 1H), 2.22 (s, 3H), 2.15 (s, 3H), 2.11 (s, 3H).

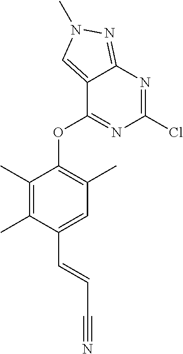 [SMILES: Cc1cc(/C=C/C#N)c(C)c(C)c1Oc1nc(Cl)nc2nn(C)cc12]

(E)-3-(4-((6-chloro-2-methyl-2H-pyrazolo[3,4-d]pyrimidin-4-yl)oxy)-2,3,5-trimethylphenyl)acrylonitrile (Intermediate 8c): (E)-3-(4-hydroxy-2,3,5-trimethylphenyl)acrylonitrile (1.48 mmol, 304 mg) was used in place of (E)-3-(4-hydroxy-3,5-dimethyl-2-(trifluoromethyl)phenyl)acrylonitrile to afford the title compound. MS (m/z) 354.13 [M+H]+

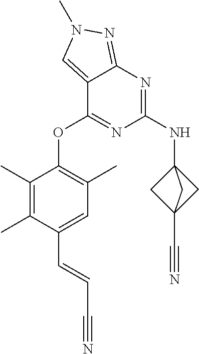 [SMILES: Cc1cc(/C=C/C#N)c(C)c(C)c1Oc1nc(NC23CC(C#N)(C2)C3)nc2nn(C)cc12]

(E)-3-((4-(4-(2-cyanovinyl)-2,3,6-trimethylphenoxy)-2-methyl-2H-pyrazolo[3,4-d]pyrimidin-6-yl)amino)bicyclo[1.1.1]pentane-1-carbonitrile—Compound 8: Intermediate 8c (0.17 mmol, 57.2 mg) was used in place of Intermediate 4a, and more 3-aminobicyclo[1.1.1]pentane-1-carbonitrile; hydrochloride (0.424 mmol, 61.3 mg) was used. Furthermore, after heating overnight, water (1 mL) was added to the mixture which was extracted with ethyl acetate (3×1 mL). The organic layers were combined and concentrated in vacuo. The resulting residue was purified by preparative HPLC (0-100% MeCN in water, 0.1% TFA) to afford the title compound. MS (m/z) 426.20 [M+H]+. ¹H NMR (400 MHz, Acetonitrile-d3) δ 8.29 (s, 1H), 7.89 (d, J=16.5 Hz, 1H), 7.44 (s, 1H), 5.99 (d, J=16.5 Hz, 1H), 4.07 (s, 3H), 2.34 (s, 3H), 2.11 (s, 3H), 2.08 (br s, 6H), 2.07 (s, 3H).

### Example 9

### (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile—Compound 9

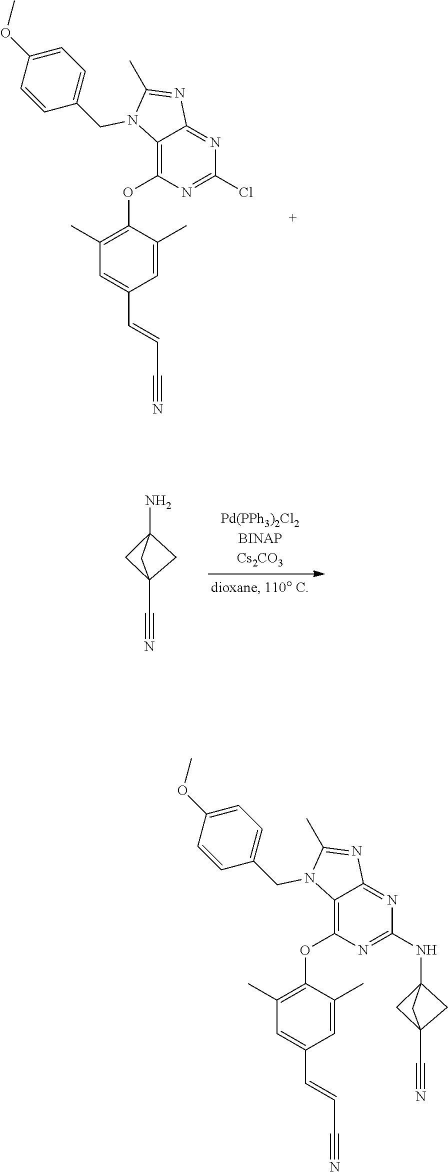 [SMILES: COc1ccc(Cn2c(C)nc3nc(Cl)nc(Oc4c(C)cc(/C=C/C#N)cc4C)c32)cc1 ; N#CC12CC(N)(C1)C2 ; COc1ccc(Cn2c(C)nc3nc(NC45CC(C#N)(C4)C5)nc(Oc4c(C)cc(/C=C/C#N)cc4C)c32)cc1]

(E)-3-[4-[2-chloro-7-[(4-methoxyphenyl)methyl]-8-methyl-purin-6-yl]oxy-3,5-dimethyl-phenyl]prop-2-enenitrile (48.0 mg, 0.104 mmol), 3-aminobicyclo[1.1.1]pentane-1-carbonitrile (13.5 mg, 0.125 mmol), Bis(triphenylphosphine)palladium chloride (7.3 mg, 0.0104 mmol), rac-BINAP (6.5 mg, 0.0104 mmol), and cesium carbonate (102 mg, 0.313 mmol) were combined in a microwave vial and dioxane (0.75 mL) was added. The mixture was degassed with N₂ and then the vial sealed and heated to 110° C. for 8 h. The mixture was then cooled to room temperature and filtered through celite, washing with EtOAc. The filtrate was concentrated in vacuo. The resulting residue was purified by silica gel chromatography (0-20% MeOH/DCM) to afford the title compound. MS (m/z) 532.1 [M+H]+.

### Example 10

### (E)-3-((4-(4-(2-cyanovinyl)-2,6-dimethylphenoxy)-1H-pyrazolo[3,4-d]pyrimidin-6-yl)amino)bicyclo[1.1.1]pentane-1-carbonitrile—Compound 10

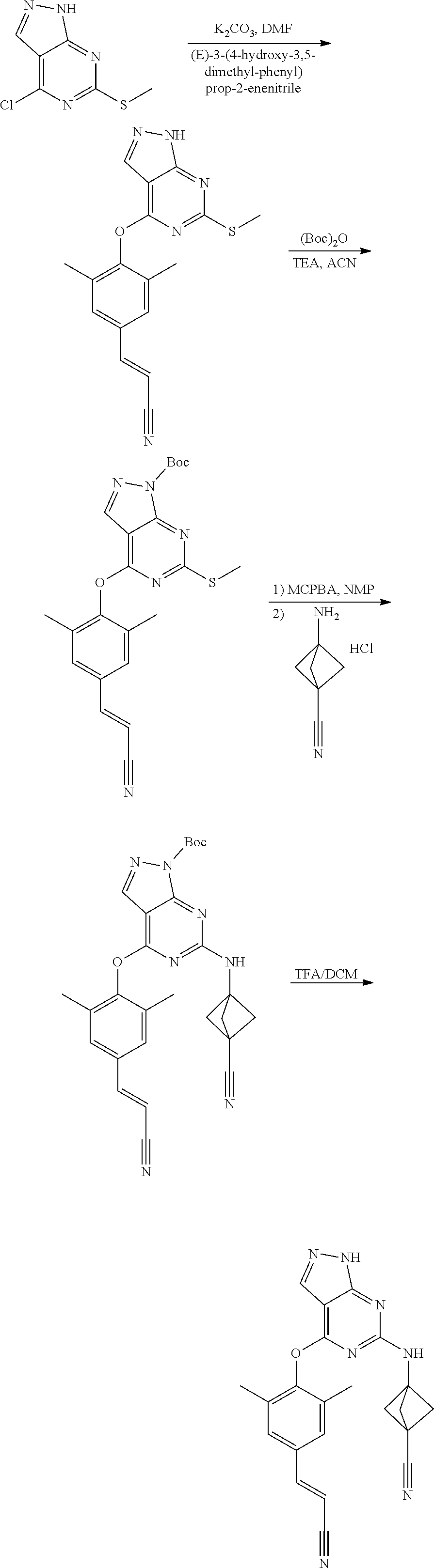

Step 1: Preparation of (E)-3-(3,5-dimethyl-4-((6-(methylthio)-1H-pyrazolo[3,4-d]pyrimidin-4-yl)oxy)phenyl)acrylonitrile (Intermediate 10a): To a solution of 4-chloro-6-methylsulfanyl-1H-pyrazolo[3,4-d]pyrimidine (348 mg, 1.73 mmol) in DMF (10 mL) was added potassium carbonate (470 mg, 3.40 mmol) and (E)-3-(4-hydroxy-3,5-dimethyl-phenyl)prop-2-enenitrile (300 mg, 1.73 mmol). The mixture was stirred at room temperature overnight and then heated at 50° C. for 3 hours. To the mixture was added water and the resulting precipitate was filtered off and dissolved in EtOAc and DCM. The organic solution was dried over MgSO4, filtered, then concentrated in vacuo onto silica gel and subjected to silica gel flash column chromatography (0-100% hexane/EtOAc) to afford the title compound. MS (m/z) 338.15 [M+H]+.

Step 2: preparation of tert-butyl (E)-4-(4-(2-cyanovinyl)-2,6-dimethylphenoxy)-6-(methylthio)-1H-pyrazolo[3,4-d]pyrimidine-1-carboxylate (Intermediate 10b): To a solution of (E)-3-(3,5-dimethyl-4-((6-(methylthio)-1H-pyrazolo[3,4-d]pyrimidin-4-yl)oxy)phenyl)acrylonitrile (50 mg, 0.15 mmol) in acetonitrile (2 mL) was added triethylamine (0.104 mL, 0.74 mmol), di-tert-butyl dicarbonate (64.7 mg, 0.296 mmol) and 4-dimethylaminopyridine (2.0 mg, 0.015 mmol). The mixture was stirred at room temperature overnight. Then the mixture was concentrated in vacuo and the resulting residue was subjected to silica gel flash column chromatography (0-100% hexane/EtOAc) to yield the title compound. MS (m/z) 437.83 [M+H]+.

Step 3: Preparation of tert-butyl (E)-6-((3-cyanobicyclo[1.1.1]pentan-1-yl)amino)-4-(4-(2-cyanovinyl)-2,6-dimethylphenoxy)-1H-pyrazolo[3,4-d]pyrimidine-1-carboxylate (Intermediate 10c): to a solution of tert-butyl (E)-4-(4-(2-cyanovinyl)-2,6-dimethylphenoxy)-6-(methylthio)-1H-pyrazolo[3,4-d]pyrimidine-1-carboxylate (30 mg, 0.068 mmol) in NMP (2 mL) was added 3-chloroperoxybenzoic acid (31.6 mg, 0.137 mmol). The mixture was stirred at room temperature overnight. To this mixture was added potassium carbonate (66 mg, 0.48 mmol) and 3-aminobicyclo[1.1.1]pentane-1-carbonitrile; hydrochloride (30 mg, 0.206 mmol). The mixture was stirred at room temperature for 4 hours. Water was added to the mixture which was subsequently extracted three times with EtOAc. The organic phase was combined, dried over MgSO4, filtered, concentrated in vacuo, and subjected to silica gel flash column chromatography (0-100% hexane/EtOAc) to afford the title compound. MS (m/z) 497.67 [M+H]+.

Step 4: Preparation of (E)-3-((4-(4-(2-cyanovinyl)-2,6-dimethylphenoxy)-1H-pyrazolo[3,4-d]pyrimidin-6-yl)amino)bicyclo[1.1.1]pentane-1-carbonitrile—Compound 10: tert-butyl (E)-6-((3-cyanobicyclo[1.1.1]pentan-1-yl)amino)-4-(4-(2-cyanovinyl)-2,6-dimethylphenoxy)-1H-pyrazolo[3,4-d]pyrimidine-1-carboxylate was suspended in a 1:1 DCM/TFA solution (1 mL) and stirred at room temperature for 2 hours. The mixture was then concentrated in vacuo and subjected to reverse phase HPLC (5-100% acetonitrile/water, containing 0.1% TFA). The fraction was dissolved in DCM, neutralized by saturated aqueousNaHCO3, the organic phase was separated, dried over MgSO4, filtered, concentrated in vacuo and subjected to silica gel flash column chromatography (0-100% hexane/EtOAc then 0-10% DCM/MeOH) yield the title compound. MS (m/z) 398.22 [M+H]⁺. ¹H NMR (400 MHz, MeOD) δ 7.97 (s, 1H), 7.54 (d, J=16.7 Hz, 1H), 7.48 (s, 2H), 6.24 (d, J=16.6 Hz, 1H), 2.17 (s, 12H).

### Example 11

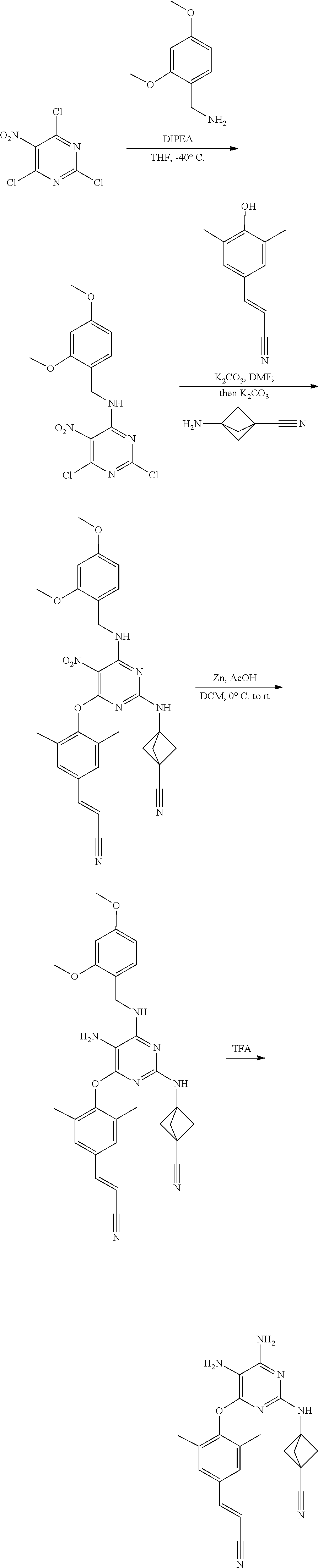

### Step 1, Preparation of 2,6-dichloro-N-(2,4-dimethoxybenzyl)-5-nitropyrimidin-4-amine (Intermediate 11a)

To a solution of 2,4,6-trichloro-5-nitro-pyrimidine (5.00 g, 21.9 mmol) in THE (100 mL) at −40° C. was added (2,4-dimethoxyphenyl)methanamine (3.29 mL, 21.9 mmol) followed by DIPEA (4.58 mL, 26.3 mmol), over the course of 15 min. The mixture was stirred at −40° C. for 30 min, then saturated NH₄Cl aqueous solution was added and the aqueous phase extracted twice with EtOAc. The combined organics were dried over MgSO₄, filtered, and concentrated in vacuo. The resulting residue was purified by silica gel chromatography (0-100% EtOAc/hexanes) to afford the title compound. MS (m/z): 358.9 [M+H]+.

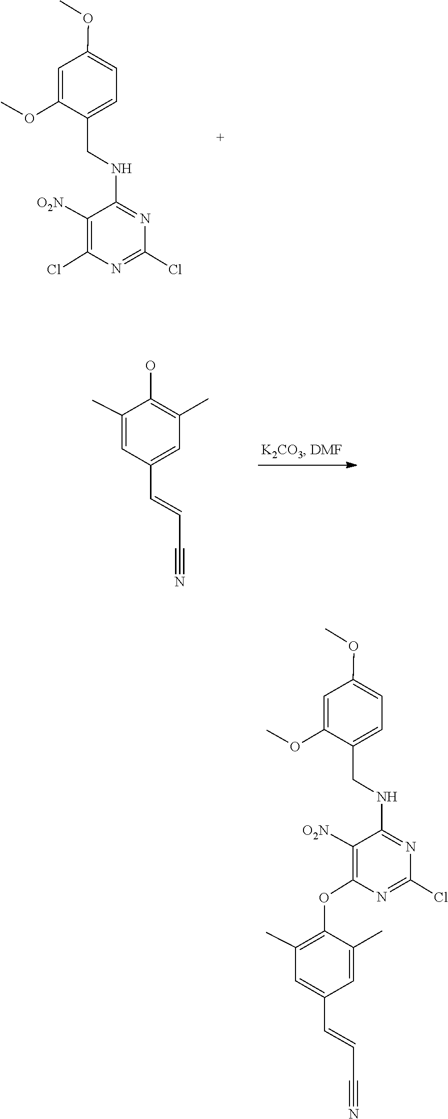

### Step 2, preparation of (E)-3-((4-(4-(2-cyanovinyl)-2,6-dimethylphenoxy)-6-((2,4-dimethoxybenzyl)amino)-5-nitropyrimidin-2-yl)amino)bicyclo[1.1.1]pentane-1-carbonitrile (Intermediate 11b)

A mixture of 2,6-dichloro-N-(2,4-dimethoxybenzyl)-5-nitropyrimidin-4-amine (1490 mg, 8.60 mmol), (E)-3-(4-hydroxy-3,5-dimethyl-phenyl)prop-2-enenitrile (1490 mg, 8.60 mmol), and potassium carbonate (5945 mg, 43.0 mmol) in DMF (45 mL) was stirred at room temperature overnight. 3-aminobicyclo[1.1.1]pentane-1-carbonitrile (1396 mg, 12.9 mmol) was then added, followed by potassium carbonate (1189 mg, 8.61 mmol), and the mixture stirred at room temperature overnight. The mixture was then diluted with EtOAc and washed twice with brine. The organic phase was dried over MgSO₄, filtered, and concentrated in vacuo. The resulting residue was purified by silica gel chromatography (0-100% EtOAc/hexanes) to afford the title compound. MS (m/z) 568.1 [M+H]+.

### Step 3, preparation (E)(E)-3-((5-amino-4-(4-(2-cyanovinyl)-2,6-dimethylphenoxy)-6-((2,4-dimethoxybenzyl)amino)pyrimidin-2-yl)amino)bicyclo[1.1.1]pentane-1-carbonitrile (Intermediate 11c)

To a solution of (E)-3-((4-(4-(2-cyanovinyl)-2,6-dimethylphenoxy)-6-((2,4-dimethoxybenzyl)amino)-5-nitropyrimidin-2-yl)amino)bicyclo[1.1.1]pentane-1-carbonitrile (1.38 g, 2.44 mmol) in DCM (25 mL) at 0° C. was added zinc dust (2.39 g, 36.5 mmol), followed by acetic acid (1.05 mL, 18.3 mmol) (steadily dropwise). The mixture was stirred for 1 h, then filtered through celite to remove zinc, and the filtrate concentrated in vacuo. The resulting residue was diluted with EtOAc, then basified with saturated aqueous NaHCO₃, layers separated, and the aqueous phase extracted with additional EtOAc. The combined organics were dried over MgSO₄, filtered, and concentrated in vacuo. The resulting residue was used without further purification. MS (m/z) 538.2 [M+H]+.

### Step 4, preparation of (E)-3-((4,5-diamino-6-(4-(2-cyanovinyl)-2,6-dimethylphenoxy)pyrimidin-2-yl)amino)bicyclo[1.1.1]pentane-1-carbonitrile (Intermediate 11d)

(E)-3-((4,5-diamino-6-(4-(2-cyanovinyl)-2,6-dimethylphenoxy)pyrimidin-2-yl)amino)bicyclo[1.1.1]pentane-1-carbonitrile was prepared using the procedure described for the synthesis of (E)-3-(4-((2-chloro-8-methyl-7H-purin-6-yl)oxy)-3,5-dimethylphenyl)acrylonitrile with the following modifications: (E)-3-((4-(4-(2-cyanovinyl)-2,6-dimethylphenoxy)-6-((2,4-dimethoxybenzyl)amino)-5-nitropyrimidin-2-yl)amino)bicyclo[1.1.1]pentane-1-carbonitrile was used in place of (E)-3-(4-((2-chloro-7-(2,4-dimethoxybenzyl)-8-methyl-7H-purin-6-yl)oxy)-3,5-dimethylphenyl)acrylonitrile. MS (m/z): 388.1 [M+H]+.

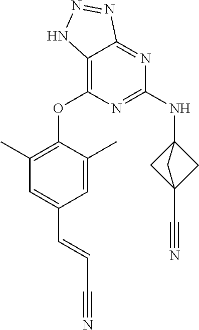 [SMILES: Cc1cc(/C=C/C#N)cc(C)c1Oc1nc(NC23CC(C#N)(C2)C3)nc2nn[nH]c12]

(E)-3-((7-(4-(2-cyanovinyl)-2,6-dimethylphenoxy)-1H-[1,2,3]triazolo[4,5-d]pyrimidin-5-yl)amino)bicyclo[1.1.1]pentane-1-carbonitrile (Intermediate Ile) was prepared as follows: A microwave vial was charged with (E)-3-((4,5-diamino-6-(4-(2-cyanovinyl)-2,6-dimethylphenoxy)pyrimidin-2-yl)amino)bicyclo[1.1.1]pentane-1-carbonitrile (50.0 mg, 0.129 mmol), which was then suspended in H₂O (0.2 mL) and acetic acid (0.2 mL). The mixture was cooled to 0° C. and NaNO₂ (16.0 mg, 0.232 mmol) was added as a solution in 0.2 mL H₂O. After 30 min, H₂O was added and the aqueous phase extracted with EtOAc. The combined organics were dried over MgSO₄, filtered, and concentrated in vacuo. The resulting residue was used without further purification. MS (m/z) 398.9 [M+H]+.

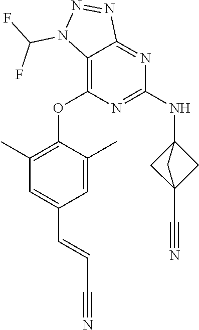 [SMILES: Cc1cc(/C=C/C#N)cc(C)c1Oc1nc(NC23CC(C#N)(C2)C3)nc2nnn(C(F)F)c12]

(E)-3-((7-(4-(2-cyanovinyl)-2,6-dimethylphenoxy)-1-(difluoromethyl)-1H-[1,2,3]triazolo[4,5-d]pyrimidin-5-yl)amino)bicyclo[1.1.1]pentane-1-carbonitrile—Compound 11 was prepared using the procedure described for the synthesis of Compound 20 with the following modifications: Iododifluoromethane (10.0% in THF) was used in place of 1,1-difluoro-2-iodo-ethane, and the reaction was stirred at room temperature (eluted as isomer 1). MS (m/z) 448.8 [M+H]+. ¹H NMR (400 MHz, DMSO-d6) δ 8.79-8.39 (m, 2H), 7.64 (d, J=16.7 Hz, 1H), 7.58 (s, 2H), 6.46 (d, J=16.7 Hz, 1H), 2.20-1.72 (m, 12H).

### Example 12

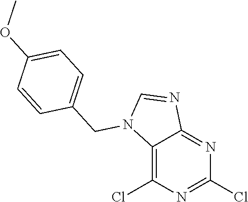 [SMILES: COc1ccc(Cn2cnc3nc(Cl)nc(Cl)c32)cc1]

2,6-dichloro-7-(4-methoxybenzyl)-7H-purine (Intermediate 12a) was prepared as follows: To mixture of 2,6-dichloro-9H-purine (1.00 g, 5.29 mmol), (4-methoxyphenyl)methanol (877 mg, 6.35 mmol), and triphenylphosphine (1804 mg, 6.88 mmol) in THE (30 mL) was added diisopropyl azodicarboxylate (1.35 mL, 6.88 mmol), and the mixture stirred at room temperature overnight. The mixture was then concentrated in vacuo and purified by silica gel chromatography (0-100% EtOAc/hexanes) to afford the title compound. MS (m/z): 309.0 [M+H]+.

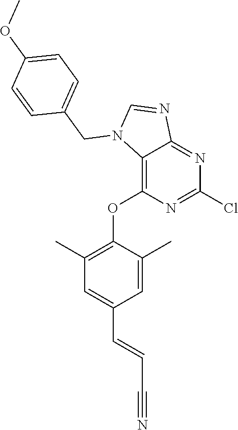 [SMILES: COc1ccc(Cn2cnc3nc(Cl)nc(Oc4c(C)cc(/C=C/C#N)cc4C)c32)cc1]

(E)-3-(4-((2-chloro-7-(4-methoxybenzyl)-7H-purin-6-yl)oxy)-3,5-dimethylphenyl)acrylonitrile (Intermediate 12b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-7-(4-methoxybenzyl)-7H-purine was used in place of 2,6-dichloro-N-[(2,4-dimethoxyphenyl)methyl]-5-nitro-pyrimidin-4-amine. MS (m/z): 446.0 [M+H]+.

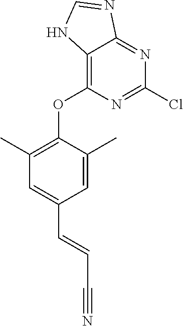 [SMILES: Cc1cc(/C=C/C#N)cc(C)c1Oc1nc(Cl)nc2nc[nH]c12]

(E)-3-(4-((2-chloro-7H-purin-6-yl)oxy)-3,5-dimethylphenyl)acrylonitrile (Intermediate 12c) was prepared as follows: A mixture of (E)-3-[4-[2-chloro-7-[(4-methoxyphenyl)methyl]purin-6-yl]oxy-3,5-dimethyl-phenyl]prop-2-enenitrile (500 mg, 1.12 mmol) in TFA (10 mL) was heated to 60° C. for 5 h. The mixture was then concentrated in vacuo, rediluted with EtOAc, and quenched with saturated NaHCO₃ aqueous solution, the layers separated, and the aqueous phase extracted with additional EtOAc. Combined organics were dried over MgSO₄, filtered, and concentrated in vacuo. The resulting residue was purified by silica gel flash column chromatography (0-20% MeOH/DCM) to afford the title compound. MS (m/z): 326.0 [M+H]+.

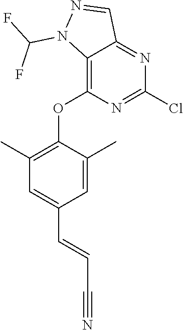 [SMILES: Cc1cc(/C=C/C#N)cc(C)c1Oc1nc(Cl)nc2cnn(C(F)F)c12]

(E)-3-(4-((2-chloro-7-(difluoromethyl)-7H-purin-6-yl)oxy)-3,5-dimethylphenyl)acrylonitrile (Intermediate 12d) was prepared as follows: To a solution of (E)-3-[4-[(2-chloro-7H-purin-6-yl)oxy]-3,5-dimethyl-phenyl]prop-2-enenitrile (50.0 mg, 0.153 mmol) in 2-MeTHF (8.00 mL) was added LiHMDS (1.00 mol/L, 0.169 mL, 0.169 mmol), and the reaction mixture stirred at room temperature for 30 min. Iododifluoromethane (10.0% in THF, 0.369 mL, 0.184 mmol) was then added, and the mixture stirred at room temperature overnight. The mixture was then quenched with saturated ammonium chloride aqueous solution and extracted with EtOAc. Combined organics were dried over MgSO₄, filtered, and concentrated in vacuo. The resulting residue was purified by silica gel flash column chromatography (0-100% EtOAc/hexanes) to afford the title compound. MS (m/z): 376.0 [M+H]+.

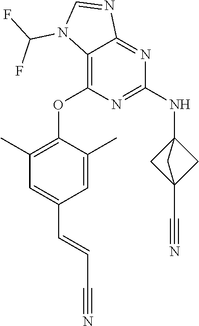 [SMILES: Cc1cc(/C=C/C#N)cc(C)c1Oc1nc(NC23CC(C#N)(C2)C3)nc2ncn(C(F)F)c12]

(E)-3-((6-(4-(2-cyanovinyl)-2,6-dimethylphenoxy)-7-(difluoromethyl)-7H-purin-2-yl)amino)bicyclo[1.1.1]pentane-1-carbonitrile—Compound 12 was prepared using the procedure described for the synthesis of Compound 4 with the following modifications: (E)-3-(4-((2-chloro-7-(difluoromethyl)-7H-purin-6-yl)oxy)-3,5-dimethylphenyl)acrylonitrile was used in place of (E)-3-(4-((6-chloro-2-methyl-2H-pyrazolo[3,4-d]pyrimidin-4-yl)oxy)-3,5-dimethyl-2-(trifluoromethyl)phenyl)acrylonitrile (Intermediate 4a). MS (m/z): 448.0 [M+H]+. ¹H NMR (400 MHz, DMSO-d6) δ 8.75 (s, 1H), 8.22-7.84 (m, 2H), 7.63 (d, J=16.7 Hz, 1H), 7.56 (s, 2H), 6.45 (d, J=16.7 Hz, 1H), 2.28-1.81 (m, 12H).

### Example 13

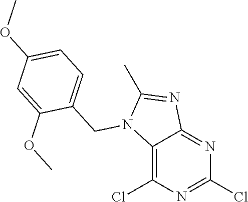 [SMILES: COc1ccc(Cn2c(C)nc3nc(Cl)nc(Cl)c32)c(OC)c1]

2,6-dichloro-7-(2,4-dimethoxybenzyl)-8-methyl-7H-purine (Intermediate 13a) was prepared using the procedure described for the synthesis of 2,6-dichloro-7-(4-methoxybenzyl)-7H-purine with the following modifications: 2,6-dichloro-8-methyl-9H-purine and (2,4-dimethoxyphenyl)methanol were used in place of 2,6-dichloro-9H-purine and (4-methoxyphenyl)methanol, respectively. MS (m/z): 352.9 [M+H]+.

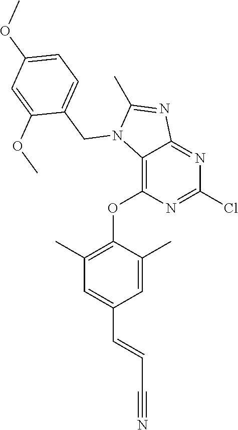 [SMILES: COc1ccc(Cn2c(C)nc3nc(Cl)nc(Oc4c(C)cc(/C=C/C#N)cc4C)c32)c(OC)c1]

(E)-3-(4-((2-chloro-7-(2,4-dimethoxybenzyl)-8-methyl-7H-purin-6-yl)oxy)-3,5-dimethylphenyl)acrylonitrile (Intermediate 13b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-7-(2,4-dimethoxybenzyl)-8-methyl-7H-purine was used in place of 2,6-dichloro-N-[(2,4-dimethoxyphenyl)methyl]-5-nitro-pyrimidin-4-amine. MS (m/z): 489.9 [M+H]+.

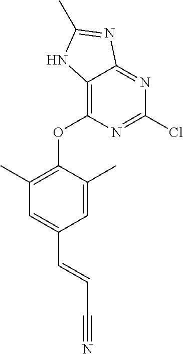 [SMILES: Cc1nc2nc(Cl)nc(Oc3c(C)cc(/C=C/C#N)cc3C)c2[nH]1]

(E)-3-(4-((2-chloro-8-methyl-7H-purin-6-yl)oxy)-3,5-dimethylphenyl)acrylonitrile (Intermediate 13c) was prepared as follows: A mixture of (E)-3-(4-((2-chloro-7-(2,4-dimethoxybenzyl)-8-methyl-7H-purin-6-yl)oxy)-3,5-dimethylphenyl)acrylonitrile (1.78 g, 3.63 mmol) in TFA (35 mL) was stirred at room temperature for 6 h. The mixture was then concentrated in vacuo, rediluted with EtOAc, and quenched with saturated NaHCO₃ aqueous solution, the layers separated, and the aqueous phase extracted with additional EtOAc.

Combined organics were dried over MgSO₄, filtered, and concentrated in vacuo. The resulting residue was used without further purification. MS (m/z): 339.9 [M+H]+.

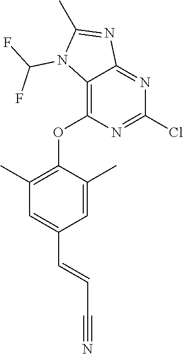 [SMILES: Cc1cc(/C=C/C#N)cc(C)c1Oc1nc(Cl)nc2nc(C)n(C(F)F)c12]

(E)-3-(4-((2-chloro-7-(difluoromethyl)-8-methyl-7H-purin-6-yl)oxy)-3,5-dimethylphenyl)acrylonitrile (Intermediate 13d) was prepared as follows: To a mixture of (E)-3-(4-((2-chloro-8-methyl-7H-purin-6-yl)oxy)-3,5-dimethylphenyl)acrylonitrile (750 mg, 2.21 mmol) and lithium carbonate (0.489 g, 6.62 mmol) in DMF (11 mL) was added ethyl 2-bromo-2,2-difluoro-acetate (0.425 mL, 3.31 mmol), and the mixture was heated to 70° C. overnight. The mixture was then diluted with EtOAc and washed twice with brine. Organics were dried over MgSO₄, filtered, and concentrated in vacuo. The resulting residue was purified by silica gel flash column chromatography (0-100% EtOAc/hexanes) to afford the title compound. MS (m/z): 389.9 [M+H]+.

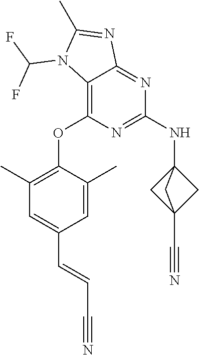 [SMILES: Cc1cc(/C=C/C#N)cc(C)c1Oc1nc(NC23CC(C#N)(C2)C3)nc2nc(C)n(C(F)F)c12]

(E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile—Compound 13 was prepared as follows: (E)-3-(4-((2-chloro-7-(difluoromethyl)-8-methyl-7H-purin-6-yl)oxy)-3,5-dimethylphenyl)acrylonitrile (104 mg, 0.267 mmol), 3-aminobicyclo[1.1.1]pentane-1-carbonitrile (72.1 mg, 0.667 mmol), rac BINAP Pd G3 (265 mg, 0.267 mmol), and cesium carbonate (261 mg, 0.800 mmol) were combined in a microwave vial and 1,4-dioxane (2.5 mL) was added. The mixture was degassed with N₂ and then the vial sealed and heated to 60° C. for 12 h. The mixture was then cooled to room temperature and concentrated in vacuo. The resulting residue was purified by preparative HPLC (0-100% MeCN/water) to afford the title compound. MS (m/z) 461.9 [M+H]+. ¹H NMR (400 MHz, DMSO-d₆) δ 8.23-7.87 (m, 2H), 7.63 (d, J=16.7 Hz, 1H), 7.56 (s, 2H), 6.44 (d, J=16.7 Hz, 1H), 2.70 (s, 3H), 2.22-1.83 (m, 12H). ¹⁹F NMR (376 MHz, DMSO-d₆) δ −92.09 (d, J=57.1 Hz).

### Example 14

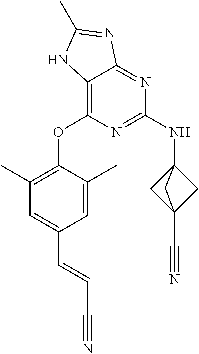 [SMILES: Cc1nc2nc(NC34CC(C#N)(C3)C4)nc(Oc3c(C)cc(/C=C/C#N)cc3C)c2[nH]1]

(E)-3-((6-(4-(2-cyanovinyl)-2,6-dimethylphenoxy)-8-methyl-7H-purin-2-yl)amino)bicyclo[1.1.1]pentane-1-carbonitrile—Compound 14 was prepared as follows: To a mixture of 3-[[4,5-diamino-6-[4-[(E)-2-cyanovinyl]-2,6-dimethyl-phenoxy]pyrimidin-2-yl]amino]bicyclo[1.1.1]pentane-1-carbonitrile (721 mg, 1.86 mmol) in DCM (9 mL) and MeOH (9 mL) was added acetaldehyde (246 mg, 5.58 mmol), followed by Ferric chloride (453 mg, 2.79 mmol) and acetic acid (0.0107 mL, 0.186 mmol). The mixture was stirred at room temperature overnight, then partitioned between water/EtOAc, the layers separated, and the aqueous phase extracted with additional EtOAc. The combined organics were dried over MgSO₄, filtered, and concentrated in vacuo. The resulting residue was purified by silica gel flash column chromatography (0-20% MeOH/DCM) to afford the title compound. MS (m/z) 411.9 [M+H]+. ¹H NMR (400 MHz, DMSO-d6) δ 12.50 (s, 1H), 7.70 (s, 1H), 7.62 (d, J=16.6 Hz, 1H), 7.54 (s, 2H), 6.43 (d, J=16.7 Hz, 1H), 2.42 (s, 3H), 2.09-2.00 (m, 12H).

### Example 15

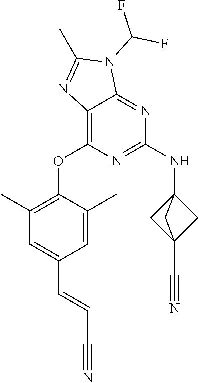 [SMILES: Cc1cc(/C=C/C#N)cc(C)c1Oc1nc(NC23CC(C#N)(C2)C3)nc2c1nc(C)n2C(F)F]

(E)-3-((6-(4-(2-cyanovinyl)-2,6-dimethylphenoxy)-9-(difluoromethyl)-8-methyl-9H-purin-2-yl)amino)bicyclo[1.1.1]pentane-1-carbonitrile—Compound 15 was prepared as follows: (E)-3-((6-(4-(2-cyanovinyl)-2,6-dimethylphenoxy)-8-methyl-7H-purin-2-yl)amino)bicyclo[1.1.1]pentane-1-carbonitrile (9.67 mg, 0.0235 mmol), lithium carbonate (0.00521 g, 0.0705 mmol), and iododifluoromethane (10.0% in THF, 0.0706 mL, 0.0353 mmol) were combined and heated to 60° C. overnight. Additional iododifluoromethane (10.0% in THF, 0.0235 mL, 0.0118 mmol) was added and the mixture heated to 80° C. for 2 h, then to 100° C. overnight. The mixture was then diluted with DMF/H₂O and purified by preparative HPLC (0-100% MeCN/water) to afford the title compound. MS (m/z) 461.9 [M+H]+. ¹H NMR (400 MHz, Acetonitrile-d3) δ 7.70-7.40 (m, 2H), 7.39 (s, 2H), 6.25 (s, 1H), 6.06 (d, J=16.7 Hz, 1H), 2.64 (d, J=1.6 Hz, 3H), 2.13-2.04 (m, 12H). ¹⁹F NMR (376 MHz, Acetonitrile-d3) δ −99.46 (d, J=58.3 Hz).

### Example 17

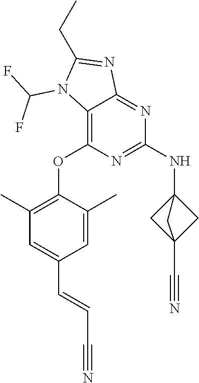 [SMILES: CCc1nc2nc(NC34CC(C#N)(C3)C4)nc(Oc3c(C)cc(/C=C/C#N)cc3C)c2n1C(F)F]

(E)-3-((6-(4-(2-cyanovinyl)-2,6-dimethylphenoxy)-7-(difluoromethyl)-8-ethyl-7H-purin-2-yl)amino)bicyclo[1.1.1]pentane-1-carbonitrile—Compound 17 was prepared using the procedure described for the synthesis of (E)-3-(4-((2-chloro-7-(difluoromethyl)-8-methyl-7H-purin-6-yl)oxy)-3,5-dimethylphenyl)acrylonitrile with the following modifications: (E)-3-((6-(4-(2-cyanovinyl)-2,6-dimethylphenoxy)-8-ethyl-7H-purin-2-yl)amino)bicyclo[1.1.1]pentane-1-carbonitrile was used in place of (E)-3-(4-((2-chloro-8-methyl-7H-purin-6-yl)oxy)-3,5-dimethylphenyl)acrylonitrile, and the reaction was heated to 60° C. MS (m/z) 475.9 [M+H]+. ¹H NMR (400 MHz, DMSO-d6) δ 8.25-7.89 (m, 2H), 7.63 (d, J=16.7 Hz, 1H), 7.55 (s, 2H), 6.44 (d, J=16.7 Hz, 1H), 3.05 (q, J=7.4 Hz, 2H), 2.24-1.85 (m, 12H), 1.33 (t, J=7.4 Hz, 3H). ¹⁹F NMR (376 MHz, DMSO-d6) δ −92.04, −92.19.

### Example 18

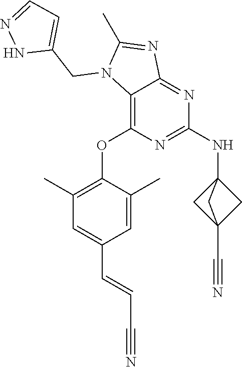 [SMILES: Cc1cc(/C=C/C#N)cc(C)c1Oc1nc(NC23CC(C#N)(C2)C3)nc2nc(C)n(Cc3ccn[nH]3)c12]

(E)-3-((7-((1H-pyrazol-5-yl)methyl)-6-(4-(2-cyanovinyl)-2,6-dimethylphenoxy)-8-methyl-7H-purin-2-yl)amino)bicyclo[1.1.1]pentane-1-carbonitrile—Compound 18 was prepared as follows: A mixture of (E)-3-((6-(4-(2-cyanovinyl)-2,6-dimethylphenoxy)-8-methyl-7H-purin-2-yl)amino)bicyclo[1.1.1]pentane-1-carbonitrile (20.0 mg, 0.0486 mmol), 5-(chloromethyl)-1H-pyrazole hydrochloride (0.015 g, 0.097 mmol), lithium carbonate (0.0108 g, 0.146 mmol), and potassium iodide (0.00403 g, 0.0243 mmol) in DMF (0.5 mL) was heated to 100° C. overnight. The mixture was then diluted with DMF/H₂O and purified by preparative HPLC (0-100% MeCN/water) to afford the title compound (eluted as isomer 1). MS (m/z) 491.9 [M+H]+. ¹H NMR (400 MHz, DMSO-d6) δ 12.76 (s, 1H), 7.73-7.56 (m, 3H), 7.51 (s, 2H), 6.41 (d, J=16.7 Hz, 1H), 6.11 (t, J=2.1 Hz, 1H), 5.51 (s, 2H), 2.57 (s, 3H), 2.12-1.90 (m, 12H).

### Example 19

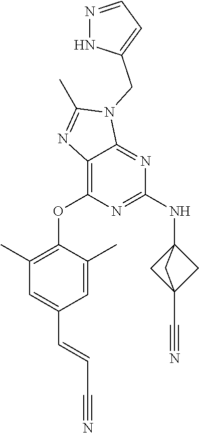 [SMILES: Cc1cc(/C=C/C#N)cc(C)c1Oc1nc(NC23CC(C#N)(C2)C3)nc2c1nc(C)n2Cc1ccn[nH]1]

(E)-3-((9-((1H-pyrazol-5-yl)methyl)-6-(4-(2-cyanovinyl)-2,6-dimethylphenoxy)-8-methyl-9H-purin-2-yl)amino)bicyclo[1.1.1]pentane-1-carbonitrile was prepared using the procedure described for the synthesis of Compound 18 (eluted as isomer 2). MS (m/z) 491.9 [M+H]+. ¹H NMR (400 MHz, Acetonitrile-d3) δ 7.56 (d, J=2.3 Hz, 1H), 7.48 (d, J=16.7 Hz, 1H), 7.38 (s, 2H), 6.32 (s, 1H), 6.24 (d, J=2.3 Hz, 1H), 6.06 (d, J=16.7 Hz, 1H), 5.30 (s, 2H), 2.63 (s, 3H), 2.17 (s, 6H), 2.11 (s, 6H).

### Example 20

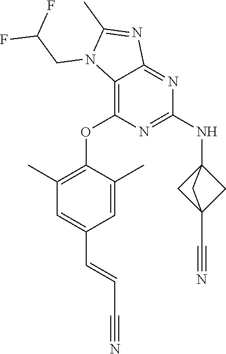 [SMILES: Cc1cc(/C=C/C#N)cc(C)c1Oc1nc(NC23CC(C#N)(C2)C3)nc2nc(C)n(CC(F)F)c12]

(E)-3-((6-(4-(2-cyanovinyl)-2,6-dimethylphenoxy)-7-(2,2-difluoroethyl)-8-methyl-7H-purin-2-yl)amino)bicyclo[1.1.1]pentane-1-carbonitrile—Compound 20 was prepared as follows: A mixture of (E)-3-((6-(4-(2-cyanovinyl)-2,6-dimethylphenoxy)-8-methyl-7H-purin-2-yl)amino)bicyclo[1.1.1]pentane-1-carbonitrile (6.00 mg, 0.0146 mmol), potassium carbonate (0.00605 g, 0.0437 mmol), and 1,1-difluoro-2-iodo-ethane (0.00193 mL, 0.0219 mmol) in DMF (0.2 mL) was heated to 60° C. overnight. The mixture was then diluted with DMF/H₂O and purified by preparative HPLC (0-100% MeCN/water) to afford the title compound (eluted as isomer 1). MS (m/z) 475.9 [M+H]+. ¹H NMR (400 MHz, DMSO-d6) δ 7.71 (s, 1H), 7.63 (d, J=16.6 Hz, 1H), 7.55 (s, 2H), 6.66-6.32 (m, 2H), 4.91-4.80 (m, 2H), 3.39 (s, 3H), 2.16-1.95 (m, 12H). ¹⁹F NMR (376 MHz, DMSO-d6) δ −123.26 (dt, J=54.3, 15.7 Hz).

### Example 21

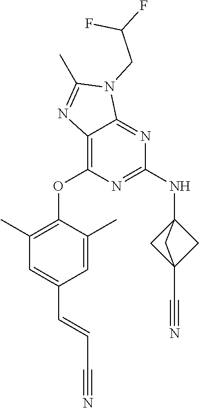 [SMILES: Cc1cc(/C=C/C#N)cc(C)c1Oc1nc(NC23CC(C#N)(C2)C3)nc2c1nc(C)n2CC(F)F]

(E)-3-((6-(4-(2-cyanovinyl)-2,6-dimethylphenoxy)-9-(2,2-difluoroethyl)-8-methyl-9H-purin-2-yl)amino)bicyclo[1.1.1]pentane-1-carbonitrile—Compound 21 was prepared using the procedure described for the synthesis of Compound 20 (eluted as isomer 2). MS (m/z) 475.9 [M+H]+. ¹H NMR (400 MHz, DMSO-d6) δ 7.95 (s, 1H), 7.61 (d, J=16.7 Hz, 1H), 7.52 (s, 2H), 6.59-6.24 (m, 2H), 4.55 (t, J=15.6 Hz, 2H), 3.31-3.26 (m, 3H), 2.38-1.62 (m, 12H). ¹⁹F NMR (376 MHz, DMSO-d6) δ −122.30 (dt, J=54.7, 15.9 Hz).

### Example 22

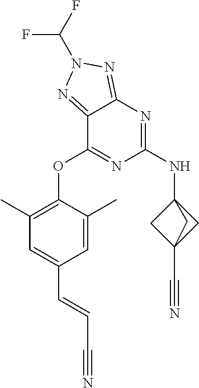 [SMILES: Cc1cc(/C=C/C#N)cc(C)c1Oc1nc(NC23CC(C#N)(C2)C3)nc2nn(C(F)F)nc12]

(E)-3-((7-(4-(2-cyanovinyl)-2,6-dimethylphenoxy)-2-(difluoromethyl)-2H-[1,2,3]triazolo[4,5-d]pyrimidin-5-yl)amino)bicyclo[1.1.1]pentane-1-carbonitrile—Compound 22 was prepared using the procedure described for the synthesis of Compound 12 (eluted as isomer 2). MS (m/z) 448.9 [M+H]+. ¹H NMR (400 MHz, DMSO-d6) δ 8.64 (d, J=79.3 Hz, 1H), 8.36 (t, J=57.4 Hz, 1H), 7.72-7.41 (m, 3H), 6.52-6.37 (m, 1H), 2.54 (s, 3H), 2.19-2.08 (m, 6H), 1.97 (s, 3H).

### Example 23

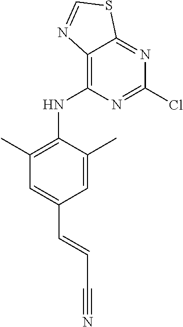 [SMILES: Cc1cc(/C=C/C#N)cc(C)c1Nc1nc(Cl)nc2scnc12]

(E)-3-(4-((5-chlorothiazolo[5,4-d]pyrimidin-7-yl)amino)-3,5-dimethylphenyl)acrylonitrile (Intermediate 23a) was prepared as follows: To a solution 5,7-dichlorothiazolo[5,4-d]pyrimidine (200 mg, 0.971 mmol) and (E)-3-(4-amino-3,5-dimethyl-phenyl)prop-2-enenitrile hydrochloride (203 mg, 0.971 mmol) in THE (4 mL) at 0° C. was added LiHMDS (1 mol/L, 2.43 mL, 2.43 mmol). The ice bath was removed and the mixture allowed to warm to rt overnight. Saturated NH₄Cl aqueous solution was then added and the aqueous phase extracted with EtOAc. The combined organics were dried over MgSO₄, filtered through celite, and concentrated in vacuo. The resulting residue was purified by silica gel chromatography (0-20% MeOH/DCM. MS (m/z) 342.0 [M+H]+.

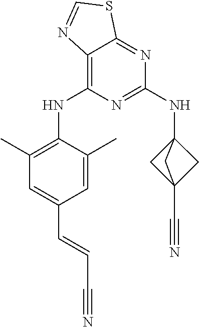 [SMILES: Cc1cc(/C=C/C#N)cc(C)c1Nc1nc(NC23CC(C#N)(C2)C3)nc2scnc12]

(E)-3-((7-((4-(2-cyanovinyl)-2,6-dimethylphenyl)amino)thiazolo[5,4-d]pyrimidin-5-yl)amino)bicyclo[1.1.1]pentane-1-carbonitrile—Compound 23 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((5-chlorothiazolo[5,4-d]pyrimidin-7-yl)amino)-3,5-dimethylphenyl)acrylonitrile was used in place of (E)-3-[4-[2-chloro-7-[(4-methoxyphenyl)methyl]-8-methyl-purin-6-yl]oxy-3,5-dimethyl-phenyl]prop-2-enenitrile, and the reaction was heated to 120° C. MS (m/z) 414.1 M+H]+. ¹H NMR (400 MHz, DMSO-d6) δ 9.51 (br s, 1H), 8.84 (s, 1H), 7.77 (s, 1H), 7.61 (d, J=16.7 Hz, 1H), 7.48 (s, 2H), 6.43 (d, J=16.7 Hz, 1H), 2.28-1.85 (m, 12H).

### Example 24

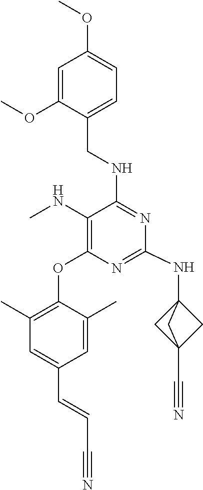 [SMILES: CNc1c(NCc2ccc(OC)cc2OC)nc(NC23CC(C#N)(C2)C3)nc1Oc1c(C)cc(/C=C/C#N)cc1C]

(E)-3-((4-(4-(2-cyanovinyl)-2,6-dimethylphenoxy)-6-((2,4-dimethoxybenzyl)amino)-5-(methylamino)pyrimidin-2-yl)amino)bicyclo[1.1.1]pentane-1-carbonitrile (Intermediate 24a) was prepared using the procedure described for the synthesis of (E)-3-(4-((2-chloro-7-(difluoromethyl)-8-methyl-7H-purin-6-yl)oxy)-3,5-dimethylphenyl)acrylonitrile with the following modifications: (E)-3-((5-amino-4-(4-(2-cyanovinyl)-2,6-dimethylphenoxy)-6-((2,4-dimethoxybenzyl)amino)pyrimidin-2-yl)amino)bicyclo[1.1.1]pentane-1-carbonitrile and iodomethane (1 equiv) were used in place of (E)-3-(4-((2-chloro-8-methyl-7H-purin-6-yl)oxy)-3,5-dimethylphenyl)acrylonitrile and ethyl 2-bromo-2,2-difluoro-acetate, respectively, and the mixture was heated at 50° C. MS (m/z) 552.1 [M+H]+.

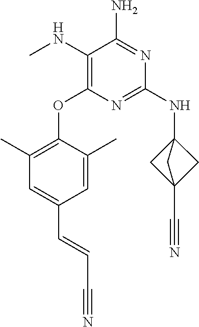 [SMILES: CNc1c(N)nc(NC23CC(C#N)(C2)C3)nc1Oc1c(C)cc(/C=C/C#N)cc1C]

(E)-3-((4-amino-6-(4-(2-cyanovinyl)-2,6-dimethylphenoxy)-5-(methylamino)pyrimidin-2-yl)amino)bicyclo[1.1.1]pentane-1-carbonitrile (Intermediate 24b) was prepared using the procedure described for the synthesis of (E)-3-(4-((2-chloro-8-methyl-7H-purin-6-yl)oxy)-3,5-dimethylphenyl)acrylonitrile with the following modifications: (E)-3-((4-(4-(2-cyanovinyl)-2,6-dimethylphenoxy)-6-((2,4-dimethoxybenzyl)amino)-5-(methylamino)pyrimidin-2-yl)amino)bicyclo[1.1.1]pentane-1-carbonitrile was used in place of (E)-3-(4-((2-chloro-7-(2,4-dimethoxybenzyl)-8-methyl-7H-purin-6-yl)oxy)-3,5-dimethylphenyl)acrylonitrile. MS (m/z) 402.1 [M+H]+.

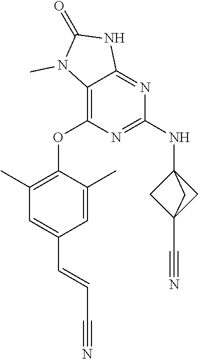 [SMILES: Cc1cc(/C=C/C#N)cc(C)c1Oc1nc(NC23CC(C#N)(C2)C3)nc2[nH]c(=O)n(C)c12]

(E)-3-((6-(4-(2-cyanovinyl)-2,6-dimethylphenoxy)-7-methyl-8-oxo-8,9-dihydro-7H-purin-2-yl)amino)bicyclo[1.1.1]pentane-1-carbonitrile Compound 24) was prepared as follows: Dioxane (0.4 mL) was added to 3-[[4-amino-6-[4-[(E)-2-cyanovinyl]-2,6-dimethyl-phenoxy]-5-(methylamino)pyrimidin-2-yl]amino]bicyclo[1.1.1]pentane-1-carbonitrile (15.0 mg, 0.0374 mmol) and carbonyldiimidazole (0.00909 g, 0.0560 mmol), and the mixture was heated to 115° C. overnight. The mixture was diluted with DMF/H₂O and purified by preparative HPLC (0-100% MeCN/water) to afford the title compound. MS (m/z) 428.0 [M+H]+. ¹H NMR (400 MHz, DMSO-d6) δ 11.57 (s, 1H), 7.67 (s, 1H), 7.61 (d, J=16.7 Hz, 1H), 7.53 (s, 2H), 6.42 (d, J=16.7 Hz, 1H), 3.43 (s, 3H), 2.11 (s, 6H), 1.99 (s, 6H).

### Example 25

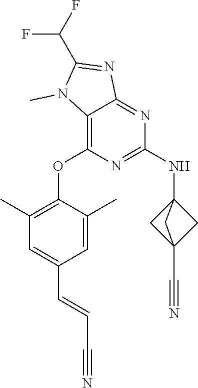 [SMILES: Cc1cc(/C=C/C#N)cc(C)c1Oc1nc(NC23CC(C#N)(C2)C3)nc2nc(C(F)F)n(C)c12]

(E)-3-((6-(4-(2-cyanovinyl)-2,6-dimethylphenoxy)-8-(difluoromethyl)-7-methyl-7H-purin-2-yl)amino)bicyclo[1.1.1]pentane-1-carbonitrile—Compound 25 was prepared as follows: A mixture of 3-[[4-amino-6-[4-[(E)-2-cyanovinyl]-2,6-dimethyl-phenoxy]-5-(methylamino)pyrimidin-2-yl]amino]bicyclo[1.1.1]pentane-1-carbonitrile (15.0 mg, 0.0374 mmol), 1-ethoxy-2,2-difluoro-ethanol (0.00741 mL, 0.0673 mmol), and acetic acid (0.00256 mL, 0.0448 mmol) in DMF (0.4 mL) was heated to 100° C. overnight. The mixture was then basified with NaHCO₃ saturated aqueous solution and purified by preparative HPLC (0-100% MeCN/water) to afford the title compound. MS (m/z) 461.9 [M+H]+. ¹H NMR (400 MHz, DMSO-d6) δ 7.91 (s, 1H), 7.64 (d, J=16.7 Hz, 1H), 7.57 (s, 2H), 7.55-7.27 (m, 1H), 6.45 (d, J=16.7 Hz, 1H), 4.12 (s, 3H), 2.11 (d, J=22.9 Hz, 12H).

### Example 26

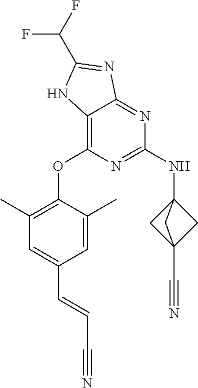 [SMILES: Cc1cc(/C=C/C#N)cc(C)c1Oc1nc(NC23CC(C#N)(C2)C3)nc2nc(C(F)F)[nH]c12]

(E)-3-((6-(4-(2-cyanovinyl)-2,6-dimethylphenoxy)-8-(difluoromethyl)-7H-purin-2-yl)amino)bicyclo[1.1.1]pentane-1-carbonitrile—Compound 26 was prepared using the procedure described for the synthesis of Compound 25 with the following modifications: (E)-3-((4,5-diamino-6-(4-(2-cyanovinyl)-2,6-dimethylphenoxy)pyrimidin-2-yl)amino)bicyclo[1.1.1]pentane-1-carbonitrile was used in place of 3-[[4-amino-6-[4-[(E)-2-cyanovinyl]-2,6-dimethyl-phenoxy]-5-(methylamino)pyrimidin-2-yl]amino]bicyclo[1.1.1]pentane-1-carbonitrile. MS (m/z) 447.9 [M+H]+. ¹H NMR (400 MHz, DMSO-d6) δ 13.67 (s, 1H), 8.07 (s, 1H), 7.63 (d, J=16.6 Hz, 1H), 7.55 (s, 2H), 7.17 (t, J=53.0 Hz, 1H), 6.44 (d, J=16.7 Hz, 1H), 2.27-1.78 (m, 12H). ¹⁹F NMR (376 MHz, DMSO-d6) δ-115.79 (d, J=52.9 Hz).

### Example 27

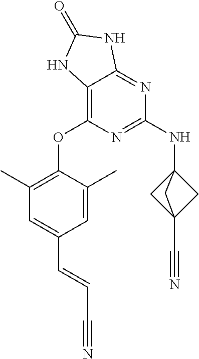 [SMILES: Cc1cc(/C=C/C#N)cc(C)c1Oc1nc(NC23CC(C#N)(C2)C3)nc2[nH]c(=O)[nH]c12]

(E)-3-((6-(4-(2-cyanovinyl)-2,6-dimethylphenoxy)-8-oxo-8,9-dihydro-7H-purin-2-yl)amino)bicyclo[1.1.1]pentane-1-carbonitrile—Compound 27 was prepared using the procedure described for the synthesis of Compound 25 with the following modifications: (E)-3-((4,5-diamino-6-(4-(2-cyanovinyl)-2,6-dimethylphenoxy)pyrimidin-2-yl)amino)bicyclo[1.1.1]pentane-1-carbonitrile was used in place of 3-[[4-amino-6-[4-[(E)-2-cyanovinyl]-2,6-dimethyl-phenoxy]-5-(methylamino)pyrimidin-2-yl]amino]bicyclo[1.1.1]pentane-1-carbonitrile, and the mixture was heated to 100° C. MS (m/z) 414.0 [M+H]+. ¹H NMR (400 MHz, DMSO-d6) δ 11.34 (s, 1H), 10.85 (s, 1H), 7.69-7.57 (m, 2H), 7.52 (s, 2H), 6.41 (d, J=16.7 Hz, 1H), 2.08 (s, 6H), 2.00 (s, 6H).

### Example 28

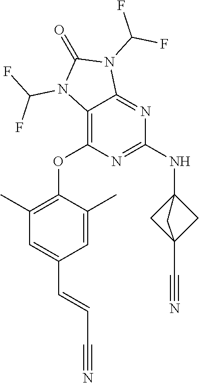 [SMILES: Cc1cc(/C=C/C#N)cc(C)c1Oc1nc(NC23CC(C#N)(C2)C3)nc2c1n(C(F)F)c(=O)n2C(F)F]

(E)-3-((6-(4-(2-cyanovinyl)-2,6-dimethylphenoxy)-7,9-bis(difluoromethyl)-8-oxo-8,9-dihydro-7H-purin-2-yl)amino)bicyclo[1.1.1]pentane-1-carbonitrile—Compound 28 was prepared using the procedure described for the synthesis of Compound 21 with the following modifications: (E)-3-((6-(4-(2-cyanovinyl)-2,6-dimethylphenoxy)-8-oxo-8,9-dihydro-7H-purin-2-yl)amino)bicyclo[1.1.1]pentane-1-carbonitrile and difluoro(iodo)methane (10.0% in THF) were used in place of (E)-3-((6-(4-(2-cyanovinyl)-2,6-dimethylphenoxy)-8-methyl-7H-purin-2-yl)amino)bicyclo[1.1.1]pentane-1-carbonitrile and 1,1-difluoro-2-iodo-ethane, respectively. MS (m/z) 513.9 [M+H]+. ¹H NMR (400 MHz, DMSO-d6) δ 8.51 (s, 1H), 7.89-7.37 (m, 5H), 6.43 (d, J=16.7 Hz, 1H), 2.19-1.76 (m, 12H). ¹⁹F NMR (376 MHz, DMSO-d6) δ −97.72 (d, J=57.2 Hz), −103.72 (d, J=57.3 Hz).

### Example 29

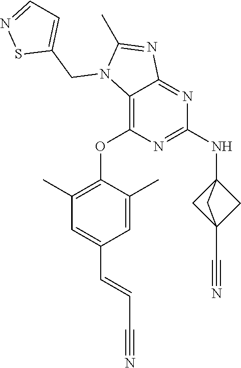 [SMILES: Cc1cc(/C=C/C#N)cc(C)c1Oc1nc(NC23CC(C#N)(C2)C3)nc2nc(C)n(Cc3ccns3)c12]

(E)-3-((6-(4-(2-cyanovinyl)-2,6-dimethylphenoxy)-7-(isothiazol-5-ylmethyl)-8-methyl-7H-purin-2-yl)amino)bicyclo[1.1.1]pentane-1-carbonitrile—Compound 29 was prepared as follows: To a mixture of (E)-3-((6-(4-(2-cyanovinyl)-2,6-dimethylphenoxy)-8-methyl-7H-purin-2-yl)amino)bicyclo[1.1.1]pentane-1-carbonitrile (27.0 mg, 0.0656 mmol), isothiazol-5-ylmethanol (0.0203 mL, 0.236 mmol), and triphenylphosphine (0.0671 g, 0.256 mmol) in THE (0.7 mL) was added diisopropyl azodicarboxylate (0.0504 mL, 0.256 mmol), and the mixture was stirred at rt overnight. The mixture was then diluted with DMF/H₂O and purified by preparative HPLC (0-100% MeCN/water) to afford the title compound (eluted as isomer 1). MS (m/z) 508.9 [M+H]+. ¹H NMR (400 MHz, DMSO-d6) δ 8.50 (d, J=1.7 Hz, 1H), 7.85 (s, 1H), 7.60 (d, J=16.7 Hz, 1H), 7.50 (s, 2H), 7.25 (d, J=1.7 Hz, 1H), 6.41 (d, J=16.7 Hz, 1H), 5.96 (s, 2H), 2.61 (s, 3H), 2.04 (s, 6H), 1.90 (s, 6H).

### Example 30

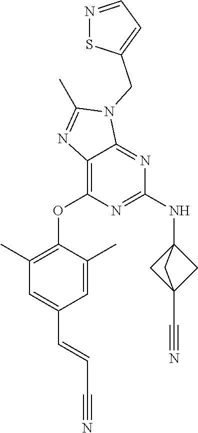 [SMILES: Cc1cc(/C=C/C#N)cc(C)c1Oc1nc(NC23CC(C#N)(C2)C3)nc2c1nc(C)n2Cc1ccns1]

(E)-3-((6-(4-(2-cyanovinyl)-2,6-dimethylphenoxy)-9-(isothiazol-5-ylmethyl)-8-methyl-9H-purin-2-yl)amino)bicyclo[1.1.1]pentane-1-carbonitrile—Compound 30 was prepared using the procedure described for the synthesis of Compound 29 (eluted as isomer 2). MS (m/z) 508.9 [M+H]+. ¹H NMR (400 MHz, DMSO-d6) δ 8.49 (d, J=1.7 Hz, 1H), 7.95 (s, 1H), 7.61 (d, J=16.7 Hz, 1H), 7.52 (s, 2H), 7.35 (s, 1H), 6.42 (d, J=16.7 Hz, 1H), 5.64 (s, 2H), 2.50 (s, 3H), 2.31-1.71 (m, 12H).

### Example 31

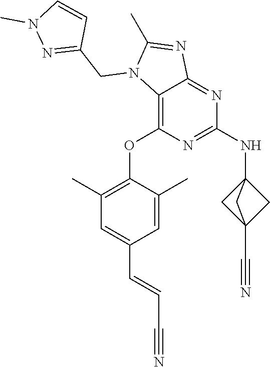 [SMILES: Cc1cc(/C=C/C#N)cc(C)c1Oc1nc(NC23CC(C#N)(C2)C3)nc2nc(C)n(Cc3ccn(C)n3)c12]

(E)-3-((6-(4-(2-cyanovinyl)-2,6-dimethylphenoxy)-8-methyl-7-((1-methyl-1H-pyrazol-3-yl)methyl)-7H-purin-2-yl)amino)bicyclo[1.1.1]pentane-1-carbonitrile—Compound 31 was prepared using the procedure described for the synthesis of Compound 29 with the following modifications: (1-methylpyrazol-3-yl)methanol was used in place of isothiazol-5-ylmethanol (title compound eluted as isomer 1). MS (m/z) 505.9 [M+H]+. ¹H NMR (400 MHz, DMSO-d6) δ 7.94 (s, 1H), 7.65 (d, J=2.2 Hz, 1H), 7.62 (d, J=16.7 Hz, 1H), 7.52 (s, 2H), 6.42 (d, J=16.7 Hz, 1H), 6.16 (d, J=2.2 Hz, 1H), 5.54 (s, 2H), 3.75 (s, 3H), 2.66 (s, 3H), 2.22-1.84 (m, 12H).

### Example 32

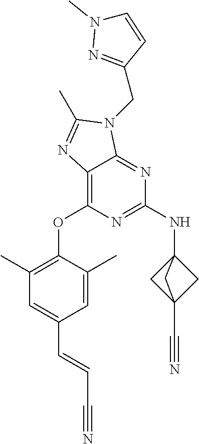 [SMILES: Cc1cc(/C=C/C#N)cc(C)c1Oc1nc(NC23CC(C#N)(C2)C3)nc2c1nc(C)n2Cc1ccn(C)n1]

(E)-3-((6-(4-(2-cyanovinyl)-2,6-dimethylphenoxy)-8-methyl-9-((1-methyl-1H-pyrazol-3-yl)methyl)-9H-purin-2-yl)amino)bicyclo[1.1.1]pentane-1-carbonitrile—Compound 32 was prepared using the procedure described for the synthesis of Compound 31 (title compound eluted as isomer 2). MS (m/z) 506.0 [M+H]+. ¹H NMR (400 MHz, DMSO-d6) δ 7.95 (s, 1H), 7.66-7.57 (m, 2H), 7.52 (s, 2H), 6.42 (d, J=16.7 Hz, 1H), 6.13 (d, J=2.2 Hz, 1H), 5.21 (s, 2H), 3.77 (s, 3H), 2.54 (s, 3H), 2.31-1.78 (m, 12H).

### Example 33

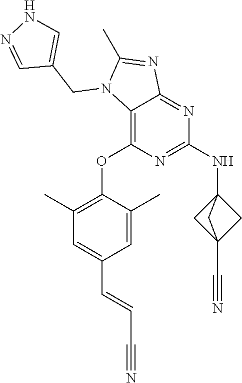 [SMILES: Cc1cc(/C=C/C#N)cc(C)c1Oc1nc(NC23CC(C#N)(C2)C3)nc2nc(C)n(Cc3cn[nH]c3)c12]

(E)-3-((7-((1H-pyrazol-4-yl)methyl)-6-(4-(2-cyanovinyl)-2,6-dimethylphenoxy)-8-methyl-7H-purin-2-yl)amino)bicyclo[1.1.1]pentane-1-carbonitrile—Compound 33 was prepared using the procedure described for the synthesis of Compound 29 with the following modifications: 1H-pyrazol-4-ylmethanol was used in place of isothiazol-5-ylmethanol (title compound eluted as isomer 1). MS (m/z) 491.9 [M+H]+. ¹H NMR (400 MHz, DMSO-d6) δ 8.09 (s, 1H), 7.67-7.58 (m, 3H), 7.54 (s, 2H), 6.43 (d, J=16.8 Hz, 1H), 5.50 (s, 2H), 2.71 (s, 3H), 2.30-1.87 (m, 12H).

### Example 34

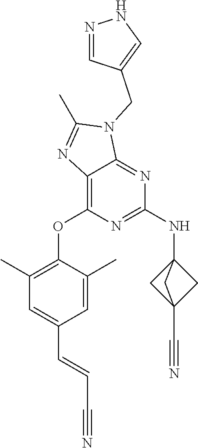 [SMILES: Cc1cc(/C=C/C#N)cc(C)c1Oc1nc(NC23CC(C#N)(C2)C3)nc2c1nc(C)n2Cc1cn[nH]c1]

(E)-3-((9-((1H-pyrazol-4-yl)methyl)-6-(4-(2-cyanovinyl)-2,6-dimethylphenoxy)-8-methyl-9H-purin-2-yl)amino)bicyclo[1.1.1]pentane-1-carbonitrile—Compound 34 was prepared using the procedure described for the synthesis of Compound 33 (title compound eluted as isomer 1). MS (m/z) 492.0 [M+H]+. ¹H NMR (400 MHz, DMSO-d6) δ 7.99 (s, 1H), 7.61 (d, J=17.2 Hz, 3H), 7.52 (s, 2H), 6.42 (d, J=16.7 Hz, 1H), 5.16 (s, 2H), 2.55 (s, 3H), 2.31-1.78 (m, 12H).

### Example 35

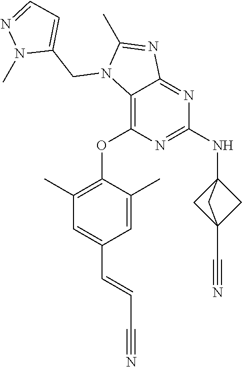 [SMILES: Cc1cc(/C=C/C#N)cc(C)c1Oc1nc(NC23CC(C#N)(C2)C3)nc2nc(C)n(Cc3ccnn3C)c12]

(E)-3-((6-(4-(2-cyanovinyl)-2,6-dimethylphenoxy)-8-methyl-7-((1-methyl-1H-pyrazol-5-yl)methyl)-7H-purin-2-yl)amino)bicyclo[1.1.1]pentane-1-carbonitrile—Compound 35 was prepared using the procedure described for the synthesis of Compound 29 with the following modifications: (2-methylpyrazol-3-yl)methanol was used in place of isothiazol-5-ylmethanol (title compound eluted as isomer 1). MS (m/z) 505.9 [M+H]+. ¹H NMR (400 MHz, DMSO-d6) δ 7.69 (s, 1H), 7.59 (d, J=16.6 Hz, 1H), 7.48 (s, 2H), 7.30 (d, J=2.0 Hz, 1H), 6.40 (d, J=16.7 Hz, 1H), 5.70-5.62 (m, 3H), 3.80 (s, 3H), 2.53 (s, 3H), 2.03 (s, 6H), 1.87 (s, 6H).

### Example 36

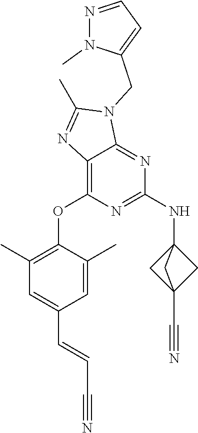 [SMILES: Cc1cc(/C=C/C#N)cc(C)c1Oc1nc(NC23CC(C#N)(C2)C3)nc2c1nc(C)n2Cc1ccnn1C]

(E)-3-((6-(4-(2-cyanovinyl)-2,6-dimethylphenoxy)-8-methyl-9-((1-methyl-1H-pyrazol-5-yl)methyl)-9H-purin-2-yl)amino)bicyclo[1.1.1]pentane-1-carbonitrile—Compound 36 was prepared using the procedure described for the synthesis of Compound 35 (title compound eluted as isomer 2). MS (m/z) 505.9 [M+H]+. ¹H NMR (400 MHz, DMSO-d6) δ 7.94 (s, 1H), 7.62 (d, J=16.7 Hz, 1H), 7.53 (s, 2H), 7.33 (d, J=1.9 Hz, 1H), 6.42 (d, J=16.7 Hz, 1H), 6.02 (s, 1H), 5.37 (s, 2H), 3.89 (s, 3H), 2.48 (s, 3H), 2.34-1.77 (m, 12H).

### Example 37

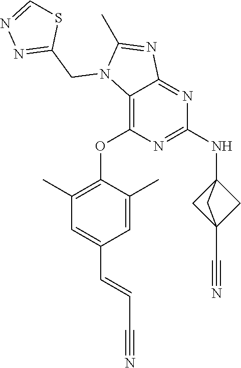 [SMILES: Cc1cc(/C=C/C#N)cc(C)c1Oc1nc(NC23CC(C#N)(C2)C3)nc2nc(C)n(Cc3nncs3)c12]

(E)-3-((7-((1,3,4-thiadiazol-2-yl)methyl)-6-(4-(2-cyanovinyl)-2,6-dimethylphenoxy)-8-methyl-7H-purin-2-yl)amino)bicyclo[1.1.1]pentane-1-carbonitrile—Compound 37 was prepared using the procedure described for the synthesis of Compound 29 with the following modifications: 1,3,4-thiadiazol-2-ylmethanol was used in place of isothiazol-5-ylmethanol (title compound eluted as isomer 1). MS (m/z) 509.9 [M+H]+. ¹H NMR (400 MHz, DMSO-d6) δ 9.61 (s, 1H), 7.77 (s, 1H), 7.60 (d, J=16.7 Hz, 1H), 7.49 (s, 2H), 6.41 (d, J=16.7 Hz, 1H), 6.10 (s, 2H), 2.61 (s, 3H), 2.04 (s, 6H), 1.89 (s, 6H).

### Example 38

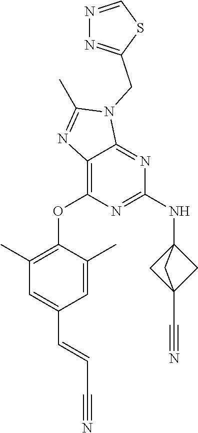 [SMILES: Cc1cc(/C=C/C#N)cc(C)c1Oc1nc(NC23CC(C#N)(C2)C3)nc2c1nc(C)n2Cc1nncs1]

(E)-3-((9-((1,3,4-thiadiazol-2-yl)methyl)-6-(4-(2-cyanovinyl)-2,6-dimethylphenoxy)-8-methyl-9H-purin-2-yl)amino)bicyclo[1.1.1]pentane-1-carbonitrile—Compound 38 was prepared using the procedure described for the synthesis of Compound 37 (title compound eluted as isomer 2). MS (m/z) 509.8 [M+H]+. ¹H NMR (400 MHz, DMSO-d6) δ 9.59 (s, 1H), 7.96 (s, 1H), 7.61 (d, J=16.7 Hz, 1H), 7.52 (s, 2H), 6.42 (d, J=16.7 Hz, 1H), 5.81 (s, 2H), 2.55 (s, 3H), 2.45-1.64 (m, 12H).

### Example 39

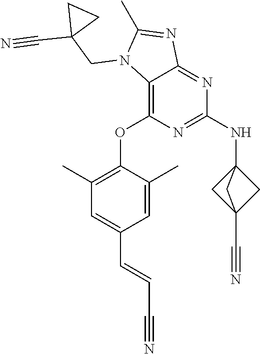 [SMILES: Cc1cc(/C=C/C#N)cc(C)c1Oc1nc(NC23CC(C#N)(C2)C3)nc2nc(C)n(CC3(C#N)CC3)c12]

(E)-3-((7-((1-cyanocyclopropyl)methyl)-6-(4-(2-cyanovinyl)-2,6-dimethylphenoxy)-8-methyl-7H-purin-2-yl)amino)bicyclo[1.1.1]pentane-1-carbonitrile—Compound 39 was prepared using the procedure described for the synthesis of Compound 18 with the following modifications: 1-(bromomethyl)cyclopropanecarbonitrile was used in place of 5-(chloromethyl)-1H-pyrazole hydrochloride (title compound eluted as isomer 1). MS (m/z) 490.9 [M+H]+. ¹H NMR (400 MHz, DMSO-d6) δ 7.75 (s, 1H), 7.63 (d, J=16.7 Hz, 1H), 7.55 (s, 2H), 6.44 (d, J=16.7 Hz, 1H), 4.56 (s, 2H), 2.65 (s, 3H), 2.17-1.96 (m, 12H), 1.46-1.39 (m, 2H), 1.36 (td, J=7.0, 6.1, 3.2 Hz, 2H).

### Example 40

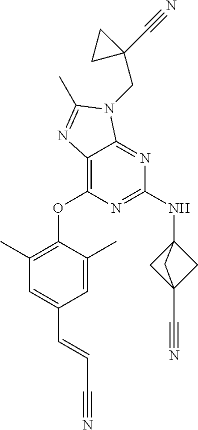 [SMILES: Cc1cc(/C=C/C#N)cc(C)c1Oc1nc(NC23CC(C#N)(C2)C3)nc2c1nc(C)n2CC1(C#N)CC1]

(E)-3-((9-((1-cyanocyclopropyl)methyl)-6-(4-(2-cyanovinyl)-2,6-dimethylphenoxy)-8-methyl-9H-purin-2-yl)amino)bicyclo[1.1.1]pentane-1-carbonitrile—Compound 40 was prepared using the procedure described for the synthesis of Compound 39 (eluted as isomer 2). MS (m/z) 491.0 [M+H]+. ¹H NMR (400 MHz, DMSO-d6) δ 7.75 (s, 1H), 7.63 (d, J=16.7 Hz, 1H), 7.55 (s, 2H), 6.44 (d, J=16.7 Hz, 1H), 4.56 (s, 2H), 2.65 (s, 3H), 2.17-1.96 (m, 12H), 1.46-1.39 (m, 2H), 1.36 (td, J=7.0, 6.1, 3.2 Hz, 2H).

### Example 41

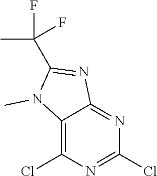 [SMILES: Cn1c(C(C)(F)F)nc2nc(Cl)nc(Cl)c21]

### 2,6-dichloro-8-(1,1-difluoroethyl)-7-methyl-7H-purine (Intermediate 41a)

To a heterogeneous, rigorously stirred, mixture of 2,6-dichloro-7-methyl-7H-purine (500 mg, 2.46 mmol, 1 equiv) and sodium 1,1-difluoroethyl sulfinate (1.12 g, 7.39 mmol, 3 equiv) in DMSO (8 mL) and water (2 mL) was added tert-butyl hydroperoxide (1.76 mL, 70 wt % in water, 12.3 mmol, 5 equiv) dropwise over 15 minutes. The reaction was stirred at room temperature for 2 hours before diluting with EtOAc/water. The resulting mixture was extracted twice with EtOAc, the combined organics were dried over MgSO₄, filtered and concentrated in vacuo. The title compound was purified by silica gel chromatography (0-50% EtOAc/hexanes). MS (m/z) 266.90/268.91 [M+H]+.

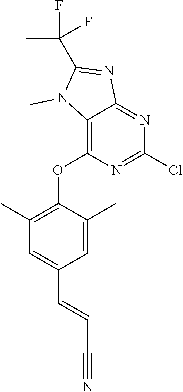 [SMILES: Cc1cc(/C=C/C#N)cc(C)c1Oc1nc(Cl)nc2nc(C(C)(F)F)n(C)c12]

(E)-3-(4-((2-chloro-8-(1,1-difluoroethyl)-7-methyl-7H-purin-6-yl)oxy)-3,5-dimethylphenyl)acrylonitrile (Intermediate 41b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-8-(1,1-difluoroethyl)-7-methyl-7H-purine was used in place of 2,6-dichloro-N-[(2,4-dimethoxyphenyl)methyl]-5-nitro-pyrimidin-4-amine. MS (m/z): 404.0 [M+H]+.

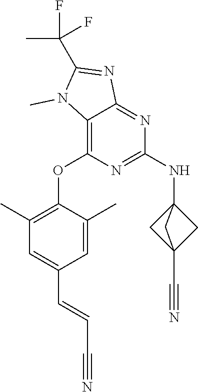 [SMILES: Cc1cc(/C=C/C#N)cc(C)c1Oc1nc(NC23CC(C#N)(C2)C3)nc2nc(C(C)(F)F)n(C)c12]

(E)-3-((6-(4-(2-cyanovinyl)-2,6-dimethylphenoxy)-8-(1,1-difluoroethyl)-7-methyl-7H-purin-2-yl)amino)bicyclo[1.1.1]pentane-1-carbonitrile—Compound 41 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((2-chloro-8-(1,1-difluoroethyl)-7-methyl-7H-purin-6-yl)oxy)-3,5-dimethylphenyl)acrylonitrile was used in place of (E)-3-[4-[2-chloro-7-[(4-methoxyphenyl)methyl]-8-methyl-purin-6-yl]oxy-3,5-dimethyl-phenyl]prop-2-enenitrile, and the mixture was heated to 120° C. for 1 h. MS (m/z) 476.1 [M+H]+. ¹H NMR (400 MHz, DMSO-d6) δ 7.86 (s, 1H), 7.63 (d, J=16.7 Hz, 1H), 7.55 (s, 2H), 6.44 (d, J=16.7 Hz, 1H), 4.15 (s, 3H), 2.27-1.93 (m, 15H).

### Example 42

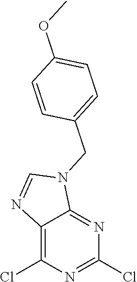 [SMILES: COc1ccc(Cn2cnc3c(Cl)nc(Cl)nc32)cc1]

2,6-dichloro-9-(4-methoxybenzyl)-9H-purine (Intermediate 42a) was prepared using the procedure described for the synthesis of 2,6-dichloro-7-(4-methoxybenzyl)-7H-purine (eluted as isomer 1). MS (m/z): 309.0 [M+H]+.

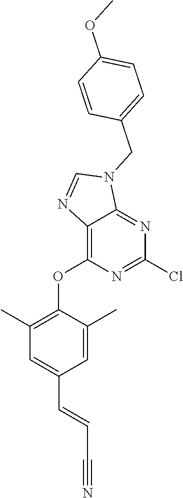 [SMILES: COc1ccc(Cn2cnc3c(Oc4c(C)cc(/C=C/C#N)cc4C)nc(Cl)nc32)cc1]

(E)-3-(4-((2-chloro-9-(4-methoxybenzyl)-9H-purin-6-yl)oxy)-3,5-dimethylphenyl)acrylonitrile (Intermediate 42b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-9-(4-methoxybenzyl)-9H-purine was used in place of 2,6-dichloro-N-[(2,4-dimethoxyphenyl)methyl]-5-nitro-pyrimidin-4-amine. MS (m/z): 446.0 [M+H]+.

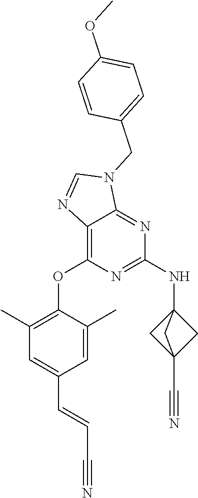 [SMILES: COc1ccc(Cn2cnc3c(Oc4c(C)cc(/C=C/C#N)cc4C)nc(NC45CC(C#N)(C4)C5)nc32)cc1]

(E)-3-((6-(4-(2-cyanovinyl)-2,6-dimethylphenoxy)-9-(4-methoxybenzyl)-9H-purin-2-yl)amino)bicyclo[1.1.1]pentane-1-carbonitrile—Compound 42 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((2-chloro-9-(4-methoxybenzyl)-9H-purin-6-yl)oxy)-3,5-dimethylphenyl)acrylonitrile was used in place of (E)-3-[4-[2-chloro-7-[(4-methoxyphenyl)methyl]-8-methyl-purin-6-yl]oxy-3,5-dimethyl-phenyl]prop-2-enenitrile, and the mixture was heated to 120° C. for 3 h. MS (m/z) 518.1 [M+H]+. ¹H NMR (400 MHz, DMSO-d6) δ 8.18 (s, 1H), 7.96 (s, 1H), 7.61 (d, J=16.7 Hz, 1H), 7.57-7.45 (m, 2H), 7.31 (d, J=8.3 Hz, 2H), 6.96-6.85 (m, 2H), 6.42 (d, J=16.7 Hz, 1H), 5.22 (s, 2H), 3.72 (s, 3H), 2.40-1.67 (m, 12H).

### Example 43

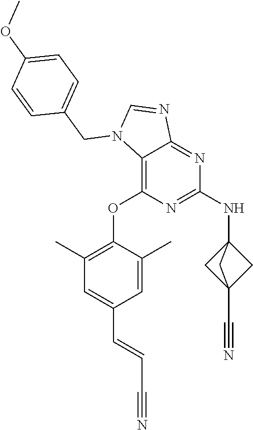 [SMILES: COc1ccc(Cn2cnc3nc(NC45CC(C#N)(C4)C5)nc(Oc4c(C)cc(/C=C/C#N)cc4C)c32)cc1]

(E)-3-((6-(4-(2-cyanovinyl)-2,6-dimethylphenoxy)-7-(4-methoxybenzyl)-7H-purin-2-yl)amino)bicyclo[1.1.1]pentane-1-carbonitrile—Compound 43 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((2-chloro-7-(4-methoxybenzyl)-7H-purin-6-yl)oxy)-3,5-dimethylphenyl)acrylonitrile was used in place of (E)-3-[4-[2-chloro-7-[(4-methoxyphenyl)methyl]-8-methyl-purin-6-yl]oxy-3,5-dimethyl-phenyl]prop-2-enenitrile, and the mixture was heated to 120° C. for 3 h. MS (m/z) 518.1 [M+H]+. ¹H NMR (400 MHz, DMSO-d6) δ 8.51 (s, 1H), 7.69 (s, 1H), 7.60 (d, J=16.6 Hz, 1H), 7.49 (s, 2H), 7.20-7.11 (m, 2H), 6.95-6.85 (m, 2H), 6.40 (d, J=16.7 Hz, 1H), 5.49 (s, 2H), 3.71 (s, 3H), 2.04 (s, 6H), 1.84 (s, 6H).

### Example 44

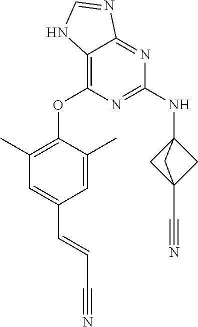 [SMILES: Cc1cc(/C=C/C#N)cc(C)c1Oc1nc(NC23CC(C#N)(C2)C3)nc2nc[nH]c12]

(E)-3-((6-(4-(2-cyanovinyl)-2,6-dimethylphenoxy)-7H-purin-2-yl)amino)bicyclo[1.1.1]pentane-1-carbonitrile—Compound 44 was prepared using the procedure described for the synthesis of (E)-3-(4-((2-chloro-7H-purin-6-yl)oxy)-3,5-dimethylphenyl)acrylonitrile with the following modifications: (E)-3-((6-(4-(2-cyanovinyl)-2,6-dimethylphenoxy)-7-(4-methoxybenzyl)-7H-purin-2-yl)amino)bicyclo[1.1.1]pentane-1-carbonitrile was used in place of (E)-3-[4-[2-chloro-7-[(4-methoxyphenyl)methyl]purin-6-yl]oxy-3,5-dimethyl-phenyl]prop-2-enenitrile, and the reaction was heated to 40° C. The title compound was purified by preparative HPLC (0-100% MeCN/water). MS (m/z) 398.1 [M+H]+. ¹H NMR (400 MHz, DMSO-d6) δ 12.72 (s, 1H), 8.01 (s, 1H), 7.71 (s, 1H), 7.62 (d, J=16.7 Hz, 1H), 7.54 (s, 2H), 6.42 (d, J=16.7 Hz, 1H), 2.18-1.96 (m, 12H).

### Example 45

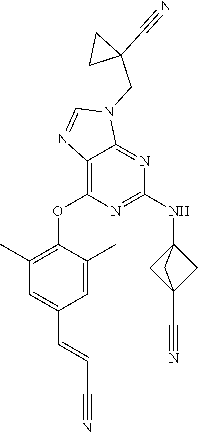 [SMILES: Cc1cc(/C=C/C#N)cc(C)c1Oc1nc(NC23CC(C#N)(C2)C3)nc2c1ncn2CC1(C#N)CC1]

(E)-3-((9-((1-cyanocyclopropyl)methyl)-6-(4-(2-cyanovinyl)-2,6-dimethylphenoxy)-9H-purin-2-yl)amino)bicyclo[1.1.1]pentane-1-carbonitrile—Compound 45 was prepared using the procedure described for the synthesis of Compound 19 with the following modifications: (E)-3-((6-(4-(2-cyanovinyl)-2,6-dimethylphenoxy)-7H-purin-2-yl)amino)bicyclo[1.1.1]pentane-1-carbonitrile and 1-(bromomethyl)cyclopropanecarbonitrile were used in place of (E)-3-((6-(4-(2-cyanovinyl)-2,6-dimethylphenoxy)-8-methyl-7H-purin-2-yl)amino)bicyclo[1.1.1]pentane-1-carbonitrile and 5-(chloromethyl)-1H-pyrazole hydrochloride, respectively. MS (m/z) 477.0 [M+H]+. ¹H NMR (400 MHz, Acetonitrile-d3) δ 7.93 (s, 1H), 7.48 (d, J=16.7 Hz, 1H), 7.39 (s, 2H), 6.19 (s, 1H), 6.06 (d, J=16.7 Hz, 1H), 4.19 (s, 2H), 2.13-2.08 (m, 12H), 1.39-1.28 (m, 4H).

### Example 46

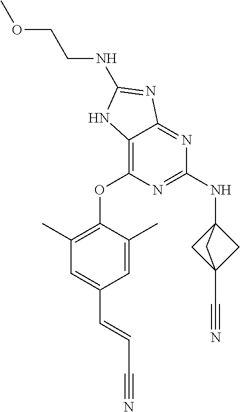 [SMILES: COCCNc1nc2nc(NC34CC(C#N)(C3)C4)nc(Oc3c(C)cc(/C=C/C#N)cc3C)c2[nH]1]

(E)-3-((6-(4-(2-cyanovinyl)-2,6-dimethylphenoxy)-8-((2-methoxyethyl)amino)-7H-purin-2-yl)amino)bicyclo[1.1.1]pentane-1-carbonitrile—Compound 46 was prepared as follows: To a mixture of (E)-3-((4,5-diamino-6-(4-(2-cyanovinyl)-2,6-dimethylphenoxy)pyrimidin-2-yl)amino)bicyclo[1.1.1]pentane-1-carbonitrile (20.0 mg, 0.0516 mmol) in pyridine (0.25 mL) was added 1-isothiocyanato-2-methoxy-ethane (0.00779 mL, 0.0671 mmol), followed by N,N′-Diisopropylcarbodiimide (0.0104 mL, 0.0671 mmol). The vial was capped and heated to 60° C. overnight. The reaction mixture was then diluted with DMF/H₂O and purified by preparative HPLC (0-100% MeCN/water with 0.1% TFA) to afford the title compound. MS (m/z) 471.1 [M+H]+. ¹H NMR (400 MHz, DMSO-d6) δ 11.18 (s, 1H), 7.61 (d, J=16.6 Hz, 1H), 7.51 (s, 2H), 7.27 (d, J=43.6 Hz, 1H), 7.04 (d, J=28.9 Hz, 1H), 6.40 (d, J=16.7 Hz, 1H), 3.48 (d, J=3.9 Hz, 4H), 3.28 (s, 3H), 2.08 (s, 6H), 2.02 (s, 6H).

### Example 47

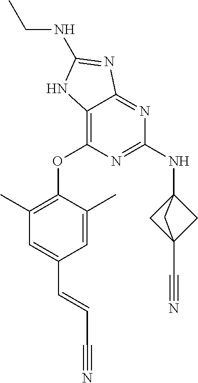 [SMILES: CCNc1nc2nc(NC34CC(C#N)(C3)C4)nc(Oc3c(C)cc(/C=C/C#N)cc3C)c2[nH]1]

(E)-3-((6-(4-(2-cyanovinyl)-2,6-dimethylphenoxy)-8-(ethylamino)-7H-purin-2-yl)amino)bicyclo[1.1.1]pentane-1-carbonitrile—Compound 47 was prepared using the procedure described for the synthesis of Compound 46 with the following modifications: isothiocyanatoethane was used in place of 1-isothiocyanato-2-methoxy-ethane. MS (m/z) 441.1 [M+H]+. ¹H NMR (400 MHz, Acetonitrile-d3) δ 7.48 (d, J=16.6 Hz, 1H), 7.40 (s, 2H), 6.07 (d, J=16.7 Hz, 1H), 3.50-3.39 (m, 2H), 2.13 (s, 6H), 2.06 (s, 6H), 1.29 (t, J=7.2 Hz, 3H).

### Example 48

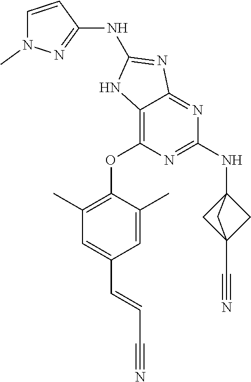 [SMILES: Cc1cc(/C=C/C#N)cc(C)c1Oc1nc(NC23CC(C#N)(C2)C3)nc2nc(Nc3ccn(C)n3)[nH]c12]

(E)-3-((6-(4-(2-cyanovinyl)-2,6-dimethylphenoxy)-8-((1-methyl-1H-pyrazol-3-yl)amino)-7H-purin-2-yl)amino)bicyclo[1.1.1]pentane-1-carbonitrile—Compound 48 was prepared using the procedure described for the synthesis of Compound 46 with the following modifications: 3-isothiocyanato-1-methyl-pyrazole was used in place of 1-isothiocyanato-2-methoxy-ethane. MS (m/z) 493.1 [M+H]+. ¹H NMR (400 MHz, DMSO-d6) δ 11.50-10.90 (m, 1H), 10.47-9.59 (m, 1H), 7.68-7.57 (m, 2H), 7.54 (s, 2H), 7.43 (d, J=20.5 Hz, 1H), 6.43 (d, J=16.7 Hz, 1H), 6.30-5.97 (m, 1H), 3.86-3.74 (m, 3H), 2.20-1.94 (m, 12H).

### Example 49

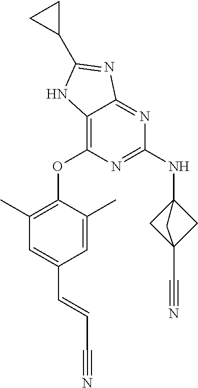 [SMILES: Cc1cc(/C=C/C#N)cc(C)c1Oc1nc(NC23CC(C#N)(C2)C3)nc2nc(C3CC3)[nH]c12]

(E)-3-((6-(4-(2-cyanovinyl)-2,6-dimethylphenoxy)-8-cyclopropyl-7H-purin-2-yl)amino)bicyclo[1.1.1]pentane-1-carbonitrile—Compound 49 was prepared as follows: To a mixture of 3-[[4,5-diamino-6-[4-[(E)-2-cyanovinyl]-2,6-dimethyl-phenoxy]pyrimidin-2-yl]amino]bicyclo[1.1.1]pentane-1-carbonitrile (11.0 mg, 0.0284 mmol) in DCM (0.5 mL) and MeOH (0.5 mL) was added cyclopropanecarbaldehyde (0.00212 mL, 0.0284 mmol), followed by acetic acid (0.000162 mL, 0.00284 mmol). The mixture was stirred at 40° C. overnight. The mixture was then basified using saturated NaHCO₃ aqueous solution, diluted with DMF/H₂O, and purified by preparative HPLC (0-100% MeCN/water) to afford the title compound. MS (m/z) 438.2 [M+H]+. ¹H NMR (400 MHz, DMSO-d6) δ 12.59 (br s, 1H), 7.67 (s, 1H), 7.61 (d, J=16.7 Hz, 1H), 7.53 (s, 2H), 6.42 (d, J=16.7 Hz, 1H), 2.18-1.93 (m, 13H), 1.09-0.98 (m, 4H).

### Example 50

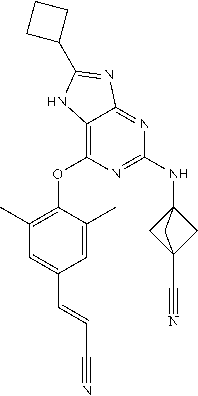 [SMILES: Cc1cc(/C=C/C#N)cc(C)c1Oc1nc(NC23CC(C#N)(C2)C3)nc2nc(C3CCC3)[nH]c12]

(E)-3-((6-(4-(2-cyanovinyl)-2,6-dimethylphenoxy)-8-cyclobutyl-7H-purin-2-yl)amino)bicyclo[1.1.1]pentane-1-carbonitrile—Compound 50 was prepared using the procedure described for the synthesis of Compound 49 with the following modifications: cyclobutanecarbaldehyde was used in place of cyclopropanecarbaldehyde. MS (m/z) 452.1 [M+H]+. ¹H NMR (400 MHz, DMSO-d6) δ 12.74 (s, 1H), 7.81 (s, 1H), 7.62 (d, J=16.7 Hz, 1H), 7.54 (s, 2H), 6.43 (d, J=16.7 Hz, 1H), 3.67 (p, J=8.7 Hz, 1H), 2.46-2.38 (m, 2H), 2.38-2.27 (m, 2H), 2.18-1.95 (m, 13H), 1.94-1.84 (m, 1H).

### Example 51

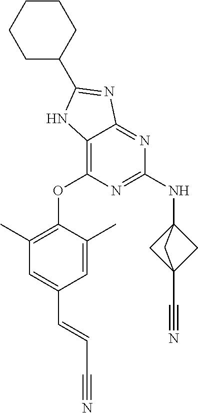 [SMILES: Cc1cc(/C=C/C#N)cc(C)c1Oc1nc(NC23CC(C#N)(C2)C3)nc2nc(C3CCCCC3)[nH]c12]

(E)-3-((6-(4-(2-cyanovinyl)-2,6-dimethylphenoxy)-8-cyclohexyl-7H-purin-2-yl)amino)bicyclo[1.1.1]pentane-1-carbonitrile—Compound 51 was prepared using the procedure described for the synthesis of Compound 49 with the following modifications: cyclohexanecarbaldehyde was used in place of cyclopropanecarbaldehyde. MS (m/z) 480.1 [M+H]+. ¹H NMR (400 MHz, DMSO-d6) δ 12.46 (s, 1H), 7.68 (s, 1H), 7.62 (d, J=16.7 Hz, 1H), 7.53 (s, 2H), 6.42 (d, J=16.6 Hz, 1H), 2.83-2.70 (m, 1H), 2.05 (d, J=13.2 Hz, 13H), 1.85-1.74 (m, 2H), 1.74-1.48 (m, 4H), 1.44-1.21 (m, 3H).

### Example 52

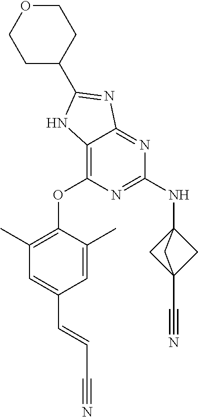 [SMILES: Cc1cc(/C=C/C#N)cc(C)c1Oc1nc(NC23CC(C#N)(C2)C3)nc2nc(C3CCOCC3)[nH]c12]

(E)-3-((6-(4-(2-cyanovinyl)-2,6-dimethylphenoxy)-8-(tetrahydro-2H-pyran-4-yl)-7H-purin-2-yl)amino)bicyclo[1.1.1]pentane-1-carbonitrile—Compound 52 was prepared as follows: To a mixture of 3-[[4,5-diamino-6-[4-[(E)-2-cyanovinyl]-2,6-dimethyl-phenoxy]pyrimidin-2-yl]amino]bicyclo[1.1.1]pentane-1-carbonitrile (30.0 mg, 0.0774 mmol) in DCM (0.4 mL) and MeOH (0.4 mL) was added tetrahydropyran-4-carbaldehyde (0.012 mL, 0.116 mmol) and acetic acid (0.000886 mL, 0.0155 mmol). The mixture was stirred at 40° C. for 1 h, then iron(III) chloride (0.0188 g, 0.116 mmol) was added. The mixture was stirred at 40° C. overnight, then basified using saturated NaHCO₃ aqueous solution, diluted with DMF/H₂O, and purified by preparative HPLC (0-100% MeCN/water) to afford the title compound. MS (m/z) 482.1 [M+H]+. ¹H NMR (400 MHz, DMSO-d6) δ 12.57 (s, 1H), 7.70 (s, 1H), 7.62 (d, J=16.6 Hz, 1H), 7.53 (s, 2H), 6.42 (d, J=16.7 Hz, 1H), 3.94 (d, J=11.5 Hz, 2H), 3.50-3.40 (m, 2H), 3.09-2.96 (m, 1H), 2.18-1.98 (m, 12H), 1.94 (d, J=12.6 Hz, 2H), 1.81 (q, J=11.1, 10.6 Hz, 2H).

### Example 53

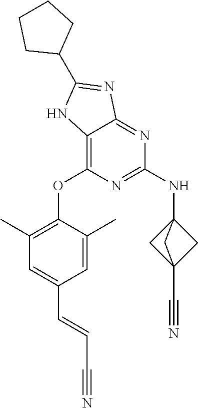 [SMILES: Cc1cc(/C=C/C#N)cc(C)c1Oc1nc(NC23CC(C#N)(C2)C3)nc2nc(C3CCCC3)[nH]c12]

(E)-3-((6-(4-(2-cyanovinyl)-2,6-dimethylphenoxy)-8-cyclopentyl-7H-purin-2-yl)amino)bicyclo[1.1.1]pentane-1-carbonitrile—Compound 53 was prepared using the procedure described for the synthesis of Compound 52 with the following modifications: cyclopentanecarbaldehyde was used in place of tetrahydropyran-4-carbaldehyde. MS (m/z) 482.1 [M+H]+. ¹H NMR (400 MHz, DMSO-d6) δ 12.57 (s, 1H), 7.70 (s, 1H), 7.62 (d, J=16.6 Hz, 1H), 7.53 (s, 2H), 6.42 (d, J=16.7 Hz, 1H), 3.94 (d, J=11.5 Hz, 2H), 3.50-3.40 (m, 2H), 3.09-2.96 (m, 1H), 2.18-1.98 (m, 12H), 1.94 (d, J=12.6 Hz, 2H), 1.81 (q, J=11.1, 10.6 Hz, 2H).

### Example 54

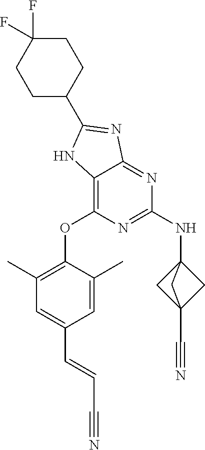 [SMILES: Cc1cc(/C=C/C#N)cc(C)c1Oc1nc(NC23CC(C#N)(C2)C3)nc2nc(C3CCC(F)(F)CC3)[nH]c12]

(E)-3-((6-(4-(2-cyanovinyl)-2,6-dimethylphenoxy)-8-(4,4-difluorocyclohexyl)-7H-purin-2-yl)amino)bicyclo[1.1.1]pentane-1-carbonitrile—Compound 54 was prepared using the procedure described for the synthesis of Compound 52 with the following modifications: 4,4-difluorocyclohexanecarbaldehyde was used in place of tetrahydropyran-4-carbaldehyde. MS (m/z) 516.1 [M+H]+. ¹H NMR (400 MHz, DMSO-d6) δ 12.61 (s, 1H), 7.70 (s, 1H), 7.62 (d, J=16.6 Hz, 1H), 7.53 (s, 2H), 6.42 (d, J=16.6 Hz, 1H), 3.04-2.91 (m, 1H), 2.20-1.74 (m, 20H).

### Example 55

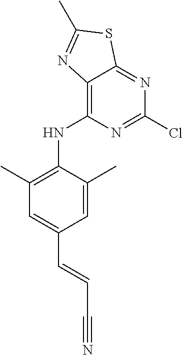 [SMILES: Cc1nc2c(Nc3c(C)cc(/C=C/C#N)cc3C)nc(Cl)nc2s1]

(E)-3-(4-((5-chloro-2-methylthiazolo[5,4-d]pyrimidin-7-yl)amino)-3,5-dimethylphenyl)acrylonitrile (Intermediate 55a) was prepared as follows: A mixture of 5,7-dichloro-2-methyl-thiazolo[5,4-d]pyrimidine (50.0 mg, 0.227 mmol), (E)-3-(4-amino-3,5-dimethyl-phenyl)prop-2-enenitrile hydrochloride (47.4 mg, 0.227 mmol), and DIPEA (0.198 mL, 1.14 mmol) in dioxane (1 mL) was stirred at 120° C. for 72 h. The mixture was then cooled to rt, concentrated in vacuo, and the resulting residue purified by silica gel chromatography (0-100% EtOAc/hexanes). MS (m/z) 356.0 [M+H]+.

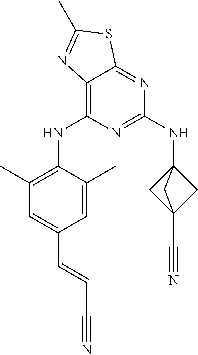 [SMILES: Cc1nc2c(Nc3c(C)cc(/C=C/C#N)cc3C)nc(NC34CC(C#N)(C3)C4)nc2s1]

(E)-3-((7-((4-(2-cyanovinyl)-2,6-dimethylphenyl)amino)-2-methylthiazolo[5,4-d]pyrimidin-5-yl)amino)bicyclo[1.1.1]pentane-1-carbonitrile—Compound 55 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((5-chloro-2-methylthiazolo[5,4-d]pyrimidin-7-yl)amino)-3,5-dimethylphenyl)acrylonitrile was used in place of (E)-3-[4-[2-chloro-7-[(4-methoxyphenyl)methyl]-8-methyl-purin-6-yl]oxy-3,5-dimethyl-phenyl]prop-2-enenitrile, and the mixture was heated to 120° C. for 3 h. MS (m/z) 428.0 [M+H]+. ¹H NMR (400 MHz, DMSO-d6) δ 9.34 (s, 1H), 7.66 (s, 1H), 7.60 (d, J=16.7 Hz, 1H), 7.47 (s, 2H), 6.42 (d, J=16.6 Hz, 1H), 2.70 (s, 3H), 2.25-1.89 (m, 12H).

### Example 56

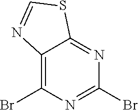 [SMILES: Brc1nc(Br)c2ncsc2n1]

5,7-dibromothiazolo[5,4-d]pyrimidine (Intermediate 56a) was prepared as follows: To a mixture of 5,7-dichlorothiazolo[5,4-d]pyrimidine (400 mg, 1.94 mmol) in MeCN (10 mL) was added bromotrimethylsilane (0.769 mL, 5.82 mmol), and the mixture heated to 60° C. for 3 h. Additional bromotrimethylsilane (0.128 g, 0.97 mmol) and the mixture heated to 60° C. for another 3 h. The mixture was then cooled to rt and saturated NaHCO₃ aqueous solution was added. The aqueous phase was extracted with EtOAc. The combined organics were dried over MgSO₄, filtered, and concentrated in vacuo. The resulting residue was used without further purification. MS (m/z) 293.8 [M+H]+.

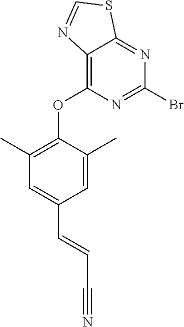 [SMILES: Cc1cc(/C=C/C#N)cc(C)c1Oc1nc(Br)nc2scnc12]

(E)-3-(4-((5-bromothiazolo[5,4-d]pyrimidin-7-yl)oxy)-3,5-dimethylphenyl)acrylonitrile (Intermediate 56b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 5,7-dibromothiazolo[5,4-d]pyrimidine was used in place of 2,6-dichloro-N-[(2,4-dimethoxyphenyl)methyl]-5-nitro-pyrimidin-4-amine. MS (m/z): 386.9 [M+H]+.

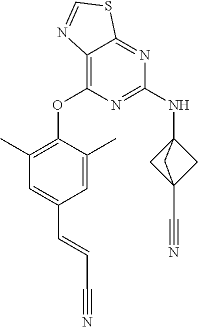 [SMILES: Cc1cc(/C=C/C#N)cc(C)c1Oc1nc(NC23CC(C#N)(C2)C3)nc2scnc12]

(E)-3-((7-(4-(2-cyanovinyl)-2,6-dimethylphenoxy)thiazolo[5,4-d]pyrimidin-5-yl)amino)bicyclo[1.1.1]pentane-1-carbonitrile—Compound 56 was prepared as follows: (E)-3-(4-((5-bromothiazolo[5,4-d]pyrimidin-7-yl)oxy)-3,5-dimethylphenyl)acrylonitrile (40.0 mg, 0.103 mmol), 3-aminobicyclo[1.1.1]pentane-1-carbonitrile (0.0168 g, 0.155 mmol), XPhos Pd G3 (0.00874 g, 0.0103 mmol), and cesium carbonate (0.0673 g, 0.207 mmol) were combined in a microwave vial and dioxane (1 mL) was added. The mixture was degassed with N₂ and then the vial sealed and heated to 60° C. for 6 h. The mixture was then cooled to room temperature and concentrated in vacuo. The resulting residue was purified by preparative HPLC (0-100% MeCN/water) to afford the title compound. MS (m/z) 415.0 [M+H]+. ¹H NMR (400 MHz, DMSO-d6) δ 9.04 (s, 1H), 8.42 (s, 1H), 7.64 (d, J=16.7 Hz, 1H), 7.56 (d, J=14.0 Hz, 2H), 6.46 (d, J=16.7 Hz, 1H), 2.22-1.80 (m, 12H).

### Example 57

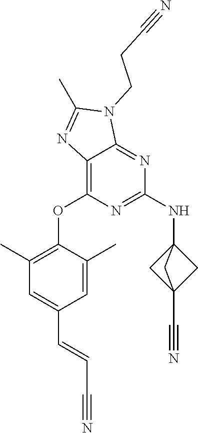 [SMILES: Cc1cc(/C=C/C#N)cc(C)c1Oc1nc(NC23CC(C#N)(C2)C3)nc2c1nc(C)n2CCC#N]

(E)-3-((9-(2-cyanoethyl)-6-(4-(2-cyanovinyl)-2,6-dimethylphenoxy)-8-methyl-9H-purin-2-yl)amino)bicyclo[1.1.1]pentane-1-carbonitrile—Compound 57 was prepared using the procedure described for the synthesis of Compound 19 with the following modifications: 3-bromopropanenitrile was used in place of 5-(chloromethyl)-1H-pyrazole hydrochloride. MS (m/z) 465.0 [M+H]+. ¹H NMR (400 MHz, DMSO-d6) δ 7.93 (s, 1H), 7.61 (d, J=16.7 Hz, 1H), 7.52 (s, 2H), 6.42 (d, J=16.7 Hz, 1H), 4.44-4.24 (m, 2H), 3.07 (t, J=6.5 Hz, 2H), 2.55 (s, 3H), 2.41-1.73 (m, 12H).

### Example 58

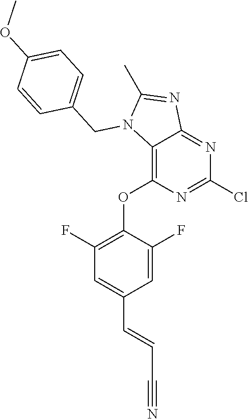 [SMILES: COc1ccc(Cn2c(C)nc3nc(Cl)nc(Oc4c(F)cc(/C=C/C#N)cc4F)c32)cc1]

(E)-3-(4-((2-chloro-7-(4-methoxybenzyl)-8-methyl-7H-purin-6-yl)oxy)-3,5-difluorophenyl)acrylonitrile (Intermediate 58a) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-7-(2,4-dimethoxybenzyl)-8-methyl-7H-purine and (E)-3-(3,5-difluoro-4-hydroxy-phenyl)prop-2-enenitrile were used in place of 2,6-dichloro-N-[(2,4-dimethoxyphenyl)methyl]-5-nitro-pyrimidin-4-amine and (E)-3-(4-hydroxy-3,5-dimethyl-phenyl)prop-2-enenitrile, respectively, and the mixture was heated to 60° C. MS (m/z): 468.0 [M+H]+.

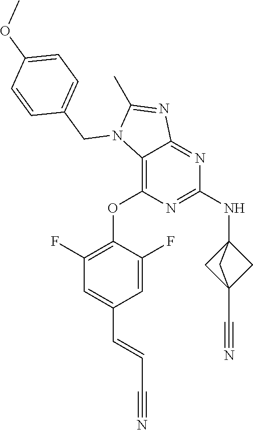 [SMILES: COc1ccc(Cn2c(C)nc3nc(NC45CC(C#N)(C4)C5)nc(Oc4c(F)cc(/C=C/C#N)cc4F)c32)cc1]

(E)-3-((6-(4-(2-cyanovinyl)-2,6-difluorophenoxy)-7-(4-methoxybenzyl)-8-methyl-7H-purin-2-yl)amino)bicyclo[1.1.1]pentane-1-carbonitrile (Intermediate 58b) was prepared using the procedure described for the synthesis of (E)-3-((6-(4-(2-cyanovinyl)-2,6-dimethylphenoxy)-7-(4-methoxybenzyl)-8-methyl-711-purin-2-yl)amino)bicyclo[1.1.1]pentane-1-carbonitrile with the following modifications: (E)-3-(4-((2-chloro-7-(4-methoxybenzyl)-8-methyl-7H-purin-6-yl)oxy)-3,5-difluorophenyl)acrylonitrile was used in place of ((E)-3-[4-[2-chloro-7-[(4-methoxyphenyl)methyl]-8-methyl-purin-6-yl]oxy-3,5-dimethyl-phenyl]prop-2-enenitrile, and the reaction was heated to 125° C. overnight. MS (m/z) 540.1 [M+H]+.

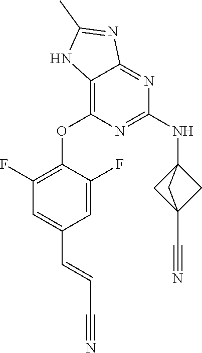 [SMILES: Cc1nc2nc(NC34CC(C#N)(C3)C4)nc(Oc3c(F)cc(/C=C/C#N)cc3F)c2[nH]1]

(E)-3-((6-(4-(2-cyanovinyl)-2,6-difluorophenoxy)-8-methyl-7H-purin-2-yl)amino)bicyclo[1.1.1]pentane-1-carbonitrile—Compound 58 was prepared using the procedure described for the synthesis of (E)-3-(4-((2-chloro-7H-purin-6-yl)oxy)-3,5-dimethylphenyl)acrylonitrile with the following modifications: (E)-3-((6-(4-(2-cyanovinyl)-2,6-difluorophenoxy)-7-(4-methoxybenzyl)-8-methyl-7H-purin-2-yl)amino)bicyclo[1.1.1]pentane-1-carbonitrile was used in place of (E)-3-[4-[2-chloro-7-[(4-methoxyphenyl)methyl]purin-6-yl]oxy-3,5-dimethyl-phenyl]prop-2-enenitrile, and the reaction was heated to 40° C. MS (m/z) 420.0 [M+H]+. ¹H NMR (400 MHz, DMSO-d6) δ 12.66 (br s, 1H), 7.76 (d, J=8.7 Hz, 2H), 7.73-7.63 (m, 2H), 6.66 (d, J=16.6 Hz, 1H), 2.40 (s, 3H), 2.15 (s, 6H).

### Example 59

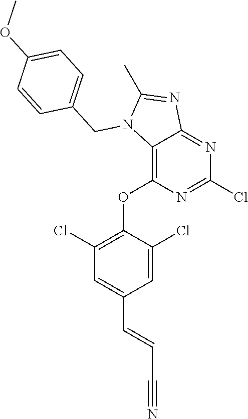 [SMILES: COc1ccc(Cn2c(C)nc3nc(Cl)nc(Oc4c(Cl)cc(/C=C/C#N)cc4Cl)c32)cc1]

(E)-3-(3,5-dichloro-4-((2-chloro-7-(4-methoxybenzyl)-8-methyl-7H-purin-6-yl)oxy)phenyl)acrylonitrile (Intermediate 59a) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,6-dichloro-7-(2,4-dimethoxybenzyl)-8-methyl-7H-purine and (E)-3-(3,5-dichloro-4-hydroxy-phenyl)prop-2-enenitrile were used in place of 2,6-dichloro-N-[(2,4-dimethoxyphenyl)methyl]-5-nitro-pyrimidin-4-amine and (E)-3-(4-hydroxy-3,5-dimethyl-phenyl)prop-2-enenitrile, respectively, and the mixture was heated to 90° C. MS (m/z): 499.9 [M+H]+.

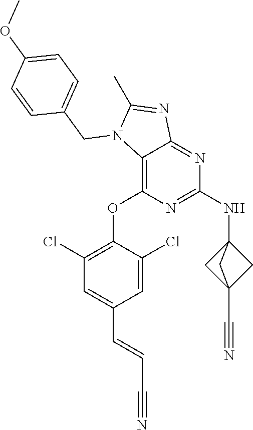 [SMILES: COc1ccc(Cn2c(C)nc3nc(NC45CC(C#N)(C4)C5)nc(Oc4c(Cl)cc(/C=C/C#N)cc4Cl)c32)cc1]

(E)-3-((6-(2,6-dichloro-4-(2-cyanovinyl)phenoxy)-7-(4-methoxybenzyl)-8-methyl-7H-purin-2-yl)amino)bicyclo[1.1.1]pentane-1-carbonitrile (Intermediate 59b) was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(3,5-dichloro-4-((2-chloro-7-(4-methoxybenzyl)-8-methyl-7H-purin-6-yl)oxy)phenyl)acrylonitrile was used in place of ((E)-3-[4-[2-chloro-7-[(4-methoxyphenyl)methyl]-8-methyl-purin-6-yl]oxy-3,5-dimethyl-phenyl]prop-2-enenitrile, and the reaction was heated to 125° C. overnight. MS (m/z) 572.0 [M+H]+.

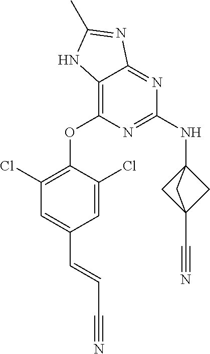 [SMILES: Cc1nc2nc(NC34CC(C#N)(C3)C4)nc(Oc3c(Cl)cc(/C=C/C#N)cc3Cl)c2[nH]1]

(E)-3-((6-(2,6-dichloro-4-(2-cyanovinyl)phenoxy)-8-methyl-7H-purin-2-yl)amino)bicyclo[1.1.1]pentane-1-carbonitrile—Compound 59 was prepared using the procedure described for the synthesis of (E)-3-(4-((2-chloro-7H-purin-6-yl)oxy)-3,5-dimethylphenyl)acrylonitrile with the following modifications: (E)-3-((6-(2,6-dichloro-4-(2-cyanovinyl)phenoxy)-7-(4-methoxybenzyl)-8-methyl-7H-purin-2-yl)amino)bicyclo[1.1.1]pentane-1-carbonitrile was used in place of (E)-3-[4-[2-chloro-7-[(4-methoxyphenyl)methyl]purin-6-yl]oxy-3,5-dimethyl-phenyl]prop-2-enenitrile, and the reaction was heated to 40° C. MS (m/z) 451.9 [M+H]+. ¹H NMR (400 MHz, Methanol-d4) δ 7.90 (s, 2H), 7.57 (d, J=16.6 Hz, 1H), 6.43 (d, J=16.7 Hz, 1H), 2.54 (s, 3H), 2.21 (s, 6H).

### Example 60

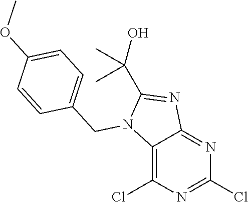 [SMILES: COc1ccc(Cn2c(C(C)(C)O)nc3nc(Cl)nc(Cl)c32)cc1]

2-(2,6-dichloro-7-(4-methoxybenzyl)-7H-purin-8-yl)propan-2-ol (Intermediate 60a) was prepared using the procedure described for the synthesis of 2,6-dichloro-7-(4-methoxybenzyl)-7H-purine with the following modifications: 2-(2,6-dichloro-7H-purin-8-yl)propan-2-ol was used in place of 2,6-dichloro-9H-purine. MS (m/z): 367.0 [M+H]+.

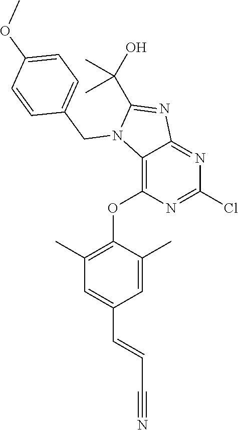 [SMILES: COc1ccc(Cn2c(C(C)(C)O)nc3nc(Cl)nc(Oc4c(C)cc(/C=C/C#N)cc4C)c32)cc1]

(E)-3-(4-((2-chloro-8-(2-hydroxypropan-2-yl)-7-(4-methoxybenzyl)-7H-purin-6-yl)oxy)-3,5-dimethylphenyl)acrylonitrile (Intermediate 60b) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2-(2,6-dichloro-7-(4-methoxybenzyl)-7H-purin-8-yl)propan-2-ol was used in place of 2,6-dichloro-N-[(2,4-dimethoxyphenyl)methyl]-5-nitro-pyrimidin-4-amine. MS (m/z): 504.1 [M+H]+.

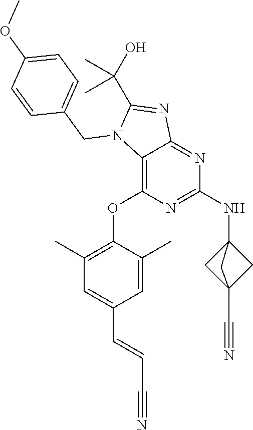 [SMILES: COc1ccc(Cn2c(C(C)(C)O)nc3nc(NC45CC(C#N)(C4)C5)nc(Oc4c(C)cc(/C=C/C#N)cc4C)c32)cc1]

(E)-3-((6-(4-(2-cyanovinyl)-2,6-dimethylphenoxy)-8-(2-hydroxypropan-2-yl)-7-(4-methoxybenzyl)-7H-purin-2-yl)amino)bicyclo[1.1.1]pentane-1-carbonitrile (Intermediate 60c) was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the following modifications: (E)-3-(4-((2-chloro-8-(2-hydroxypropan-2-yl)-7-(4-methoxybenzyl)-7H-purin-6-yl)oxy)-3, 5-dimethylphenyl)acrylonitrile was used in place of ((E)-3-[4-[2-chloro-7-[(4-methoxyphenyl)methyl]-8-methyl-purin-6-yl]oxy-3,5-dimethyl-phenyl]prop-2-enenitrile, and the reaction was heated to 120° C. for 4 h. MS (m/z) 576.2 [M+H]+.

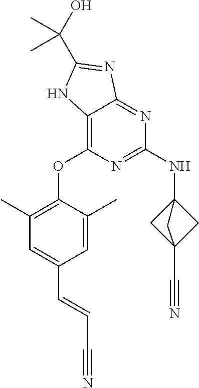 [SMILES: Cc1cc(/C=C/C#N)cc(C)c1Oc1nc(NC23CC(C#N)(C2)C3)nc2nc(C(C)(C)O)[nH]c12]

(E)-3-((6-(4-(2-cyanovinyl)-2,6-dimethylphenoxy)-8-(2-hydroxypropan-2-yl)-7H-purin-2-yl)amino)bicyclo[1.1.1]pentane-1-carbonitrile—Compound 60 was prepared using the procedure described for the synthesis of (E)-3-(4-((2-chloro-7H-purin-6-yl)oxy)-3,5-dimethylphenyl)acrylonitrile with the following modifications: (E)-3-((6-(4-(2-cyanovinyl)-2,6-dimethylphenoxy)-8-(2-hydroxypropan-2-yl)-7-(4-methoxybenzyl)-7H-purin-2-yl)amino)bicyclo[1.1.1]pentane-1-carbonitrile was used in place of (E)-3-[4-[2-chloro-7-[(4-methoxyphenyl)methyl]purin-6-yl]oxy-3,5-dimethyl-phenyl]prop-2-enenitrile, and the reaction was heated to 40° C. MS (m/z) 456.1 [M+H]+. ¹H NMR (400 MHz, DMSO-d6) δ 12.58 (s, 1H), 7.75 (s, 1H), 7.62 (d, J=16.6 Hz, 1H), 7.54 (s, 2H), 6.42 (d, J=16.7 Hz, 1H), 5.49 (s, 1H), 2.17-1.89 (m, 12H), 1.55 (s, 6H).

### Example 63

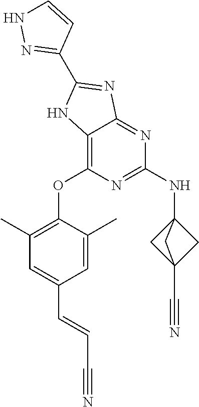 [SMILES: Cc1cc(/C=C/C#N)cc(C)c1Oc1nc(NC23CC(C#N)(C2)C3)nc2nc(-c3cc[nH]n3)[nH]c12]

(E)-3-((6-(4-(2-cyanovinyl)-2,6-dimethylphenoxy)-8-(1H-pyrazol-3-yl)-7H-purin-2-yl)amino)bicyclo[1.1.1]pentane-1-carbonitrile—Compound 63 was prepared using the procedure described for the synthesis of Compound 52 with the following modifications: 1H-pyrazole-3-carbaldehyde was used in place of tetrahydropyran-4-carbaldehyde, and the mixture was stirred at rt. MS (m/z) 464.1 [M+H]+. ¹H NMR (400 MHz, DMSO-d6) δ 13.54-12.85 (m, 2H), 7.91 (s, 1H), 7.85 (s, 1H), 7.64 (d, J=16.7 Hz, 1H), 7.56 (s, 2H), 6.89 (s, 1H), 6.44 (d, J=16.7 Hz, 1H), 2.20-1.95 (m, 12H).

### Example 64

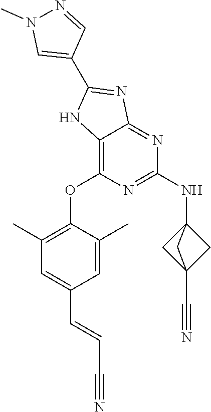 [SMILES: Cc1cc(/C=C/C#N)cc(C)c1Oc1nc(NC23CC(C#N)(C2)C3)nc2nc(-c3cnn(C)c3)[nH]c12]

(E)-3-((6-(4-(2-cyanovinyl)-2,6-dimethylphenoxy)-8-(1-methyl-1H-pyrazol-4-yl)-7H-purin-2-yl)amino)bicyclo[1.1.1]pentane-1-carbonitrile—Compound 64 was prepared using the procedure described for the synthesis of Compound 52 with the following modifications: 1-methylpyrazole-4-carbaldehyde was used in place of tetrahydropyran-4-carbaldehyde, and the mixture was stirred at rt after the addition of iron(III) chloride. MS (m/z) 478.1 [M+H]+. ¹H NMR (400 MHz, DMSO-d6) δ 12.97 (s, 1H), 8.33 (s, 1H), 8.06 (s, 1H), 7.76 (s, 1H), 7.61 (s, 1H), 7.55 (s, 2H), 6.46 (s, 1H), 3.93 (s, 3H), 2.20-1.95 (m, 12H).

### Example 65

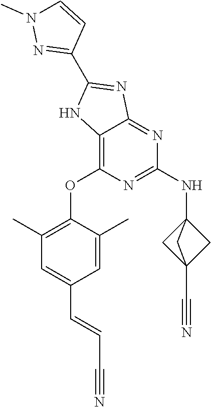 [SMILES: Cc1cc(/C=C/C#N)cc(C)c1Oc1nc(NC23CC(C#N)(C2)C3)nc2nc(-c3ccn(C)n3)[nH]c12]

(E)-3-((6-(4-(2-cyanovinyl)-2,6-dimethylphenoxy)-8-(1-methyl-1H-pyrazol-3-yl)-7H-purin-2-yl)amino)bicyclo[1.1.1]pentane-1-carbonitrile—Compound 65 was prepared using the procedure described for the synthesis of Compound 52 with the following modifications: 1-methylpyrazole-3-carbaldehyde was used in place of tetrahydropyran-4-carbaldehyde, and the mixture was stirred at rt. MS (m/z) 478.1 [M+H]+. ¹H NMR (400 MHz, DMSO-d6) δ 13.13 (s, 1H), 7.87 (s, 2H), 7.64 (d, J=16.7 Hz, 1H), 7.56 (s, 2H), 6.87 (s, 1H), 6.44 (d, J=16.7 Hz, 1H), 3.97 (s, 3H), 2.15-1.82 (m, 12H).

### Example 66

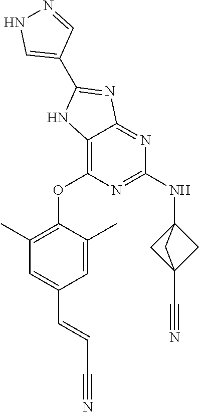 [SMILES: Cc1cc(/C=C/C#N)cc(C)c1Oc1nc(NC23CC(C#N)(C2)C3)nc2nc(-c3cn[nH]c3)[nH]c12]

(E)-3-((6-(4-(2-cyanovinyl)-2,6-dimethylphenoxy)-8-(1H-pyrazol-4-yl)-7H-purin-2-yl)amino)bicyclo[1.1.1]pentane-1-carbonitrile—Compound 66 was prepared using the procedure described for the synthesis of Compound 52 with the following modifications: 1H-pyrazole-4-carbaldehyde was used in place of tetrahydropyran-4-carbaldehyde, and the mixture was stirred at rt. MS (m/z) 464.1 [M+H]+. ¹H NMR (400 MHz, DMSO-d6) δ 13.26 (s, 1H), 12.94 (s, 1H), 8.37 (s, 1H), 8.11 (s, 1H), 7.77 (s, 1H), 7.64 (d, J=16.6 Hz, 1H), 7.55 (s, 2H), 6.44 (d, J=16.7 Hz, 1H), 2.18-1.95 (m, 12H).

### Example 67

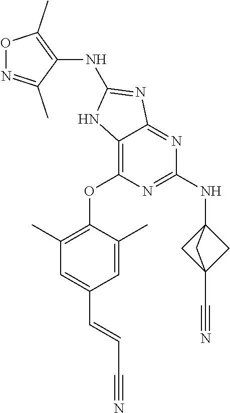 [SMILES: Cc1cc(/C=C/C#N)cc(C)c1Oc1nc(NC23CC(C#N)(C2)C3)nc2nc(Nc3c(C)noc3C)[nH]c12]

(E)-3-((6-(4-(2-cyanovinyl)-2,6-dimethylphenoxy)-8-((3,5-dimethylisoxazol-4-yl)amino)-7H-purin-2-yl)amino)bicyclo[1.1.1]pentane-1-carbonitrile—Compound 67 was prepared using the procedure described for the synthesis of Compound 46 with the following modifications: 3,5-dimethyl-4-isoxazolyl isothiocyanate was used in place of 1-isothiocyanato-2-methoxy-ethane. MS (m/z) 507.9 [M+H]+. ¹H NMR (400 MHz, DMSO-d6) δ 9.42 (s, 1H), 7.81 (s, 1H), 7.63 (d, J=16.7 Hz, 1H), 7.55 (s, 2H), 6.43 (d, J=16.7 Hz, 1H), 2.34 (s, 3H), 2.15 (s, 3H), 2.10 (s, 6H), 2.05 (s, 6H).

### Example 68

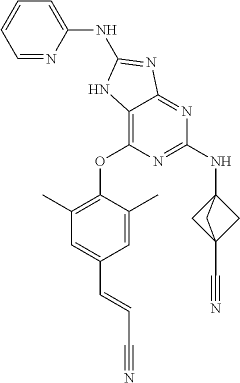 [SMILES: Cc1cc(/C=C/C#N)cc(C)c1Oc1nc(NC23CC(C#N)(C2)C3)nc2nc(Nc3ccccn3)[nH]c12]

(E)-3-((6-(4-(2-cyanovinyl)-2,6-dimethylphenoxy)-8-(pyridin-2-ylamino)-7H-purin-2-yl)amino)bicyclo[1.1.1]pentane-1-carbonitrile—Compound 68 was prepared using the procedure described for the synthesis of Compound 47 with the following modifications: 2-isothiocyanatopyridine was used in place of 1-isothiocyanato-2-methoxy-ethane. MS (m/z) 490.1 [M+H]+. ¹H NMR (400 MHz, DMSO-d6) δ 8.35 (d, J=5.2 Hz, 1H), 7.84 (t, J=7.8 Hz, 1H), 7.75 (s, 1H), 7.64 (d, J=16.7 Hz, 1H), 7.56 (s, 2H), 7.31 (d, J=8.4 Hz, 1H), 7.07 (dd, J=7.1, 5.3 Hz, 1H), 6.43 (d, J=16.8 Hz, 1H), 2.18-1.99 (m, 12H).

### Example 69

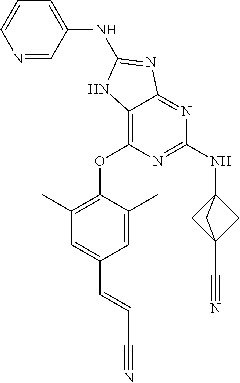 [SMILES: Cc1cc(/C=C/C#N)cc(C)c1Oc1nc(NC23CC(C#N)(C2)C3)nc2nc(Nc3cccnc3)[nH]c12]

(E)-3-((6-(4-(2-cyanovinyl)-2,6-dimethylphenoxy)-8-(pyridin-3-ylamino)-7H-purin-2-yl)amino)bicyclo[1.1.1]pentane-1-carbonitrile—Compound 69 was prepared using the procedure described for the synthesis of Compound 46 with the following modifications: 3-pyridyl isothiocyanate was used in place of 1-isothiocyanato-2-methoxy-ethane. MS (m/z) 489.9 [M+H]+. ¹H NMR (400 MHz, DMSO-d6) δ 10.34 (s, 1H), 9.24 (s, 1H), 8.52 (d, J=8.9 Hz, 1H), 8.39 (d, J=4.7 Hz, 1H), 7.77 (dd, J=8.4, 4.8 Hz, 1H), 7.70-7.59 (m, 2H), 7.55 (s, 2H), 6.43 (d, J=16.7 Hz, 1H), 2.11 (s, 6H), 2.06 (s, 6H).

### Example 70

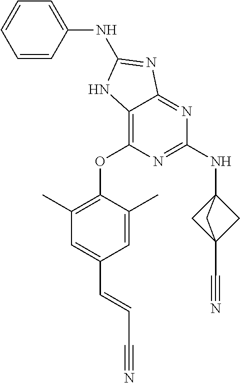 [SMILES: Cc1cc(/C=C/C#N)cc(C)c1Oc1nc(NC23CC(C#N)(C2)C3)nc2nc(Nc3ccccc3)[nH]c12]

(E)-3-((6-(4-(2-cyanovinyl)-2,6-dimethylphenoxy)-8-(phenylamino)-7H-purin-2-yl)amino)bicyclo[1.1.1]pentane-1-carbonitrile—Compound 70 was prepared using the procedure described for the synthesis of Compound 46 with the following modifications: isothiocyanatobenzene was used in place of 1-isothiocyanato-2-methoxy-ethane. MS (m/z) 489.1 [M+H]+. ¹H NMR (400 MHz, DMSO-d6) δ 9.82 (s, 1H), 7.80-7.59 (m, 4H), 7.55 (s, 2H), 7.36 (t, J=7.9 Hz, 2H), 7.04 (t, J=7.2 Hz, 1H), 6.43 (d, J=16.7 Hz, 1H), 2.21-1.93 (m, 12H).

### Example 71

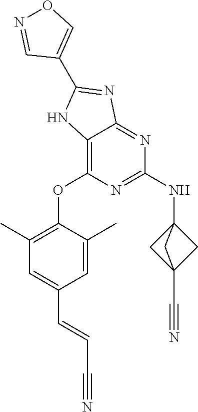 [SMILES: Cc1cc(/C=C/C#N)cc(C)c1Oc1nc(NC23CC(C#N)(C2)C3)nc2nc(-c3cnoc3)[nH]c12]

### (E)-3-((6-(4-(2-cyanovinyl)-2,6-dimethylphenoxy)-8-(isoxazol-4-yl)-7H-purin-2-yl)amino)bicyclo[1.1.1]pentane-1-carbonitrile—Compound 71

(E)-3-((4,5-diamino-6-(4-(2-cyanovinyl)-2,6-dimethylphenoxy)pyrimidin-2-yl)amino)bicyclo[1.1.1]pentane-1-carbonitrile (40.0 mg, 0.103 mmol) and isoxazole-4-carbaldehyde (15.0 mg, 0.155 mmol) were combined in a vial. MeOH (0.560 mL) and DCM (0.560 mL) were added to the vial, and the mixture was stirred for 1 h at 40° C. The mixture was then concentrated to dryness in vacuo and DMF was added (1.120 mL). KI (34.3 mg, 0.206 mmol) and 12 (26.2 mg, 0.103 mmol) were added to the solution and stirred overnight at 40° C., then purified by HPLC (15-95% MeCN/H₂O) and dried overnight on the lyo to provide the title compound. ES/MS m/z: 465.1 [M+1]. ¹H NMR (400 MHz, DMSO-d6) δ 13.30 (s, 1H), 9.55 (s, 1H), 9.23 (s, 1H), 7.93 (s, 1H), 7.64 (d, J=16.6 Hz, 1H), 7.54 (d, J=13.2 Hz, 2H), 6.44 (d, J=16.7 Hz, 1H), 2.23-1.91 (m, 12H).

### Example 72

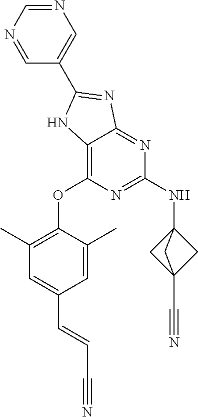 [SMILES: Cc1cc(/C=C/C#N)cc(C)c1Oc1nc(NC23CC(C#N)(C2)C3)nc2nc(-c3cncnc3)[nH]c12]

(E)-3-((6-(4-(2-cyanovinyl)-2,6-dimethylphenoxy)-8-(pyrimidin-5-yl)-7H-purin-2-yl)amino)bicyclo[1.1.1]pentane-1-carbonitrile—Compound 72 was prepared using the procedure described for the synthesis of Compound 71, but replacing isoxazole-4-carbaldehyde with pyrimidine-5-carbaldehyde, and stirred overnight at 80° C. instead of 40° C. ES/MS m/z: 476.1 [M+1]. ¹H NMR (400 MHz, DMSO-d6) δ 13.61 (s, 1H), 9.45 (s, 2H), 9.28 (s, 1H), 8.03 (s, 1H), 7.64 (d, J=16.6 Hz, 1H), 7.57 (s, 2H), 6.45 (d, J=16.7 Hz, 1H), 2.29-1.81 (m, 12H).

### Example 73

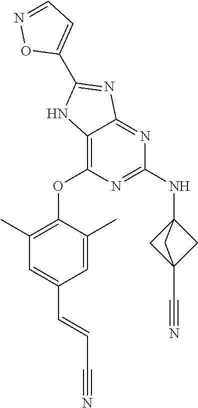 [SMILES: Cc1cc(/C=C/C#N)cc(C)c1Oc1nc(NC23CC(C#N)(C2)C3)nc2nc(-c3ccno3)[nH]c12]

(E)-3-((6-(4-(2-cyanovinyl)-2,6-dimethylphenoxy)-8-(isoxazol-5-yl)-7H-purin-2-yl)amino)bicyclo[1.1.1]pentane-1-carbonitrile—Compound 72 was prepared using the procedure described for the synthesis of Compound 71 with the following modifications: isoxazole-5-carbaldehyde was used in place of isoxazole-4-carbaldehyde. MS (m/z) 464.9 [M+H]+. ¹H NMR (400 MHz, DMSO-d6) δ 13.79 (s, 1H), 8.81 (s, 1H), 8.09 (s, 1H), 7.64 (d, J=16.7 Hz, 1H), 7.57 (s, 2H), 7.17 (s, 1H), 6.45 (d, J=16.7 Hz, 1H), 2.22-1.85 (m, 12H).

### Example 74

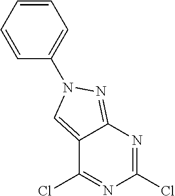 [SMILES: Clc1nc(Cl)c2cn(-c3ccccc3)nc2n1]

4,6-dichloro-2-phenyl-2H-pyrazolo[3,4-d]pyrimidine was prepared as follows:

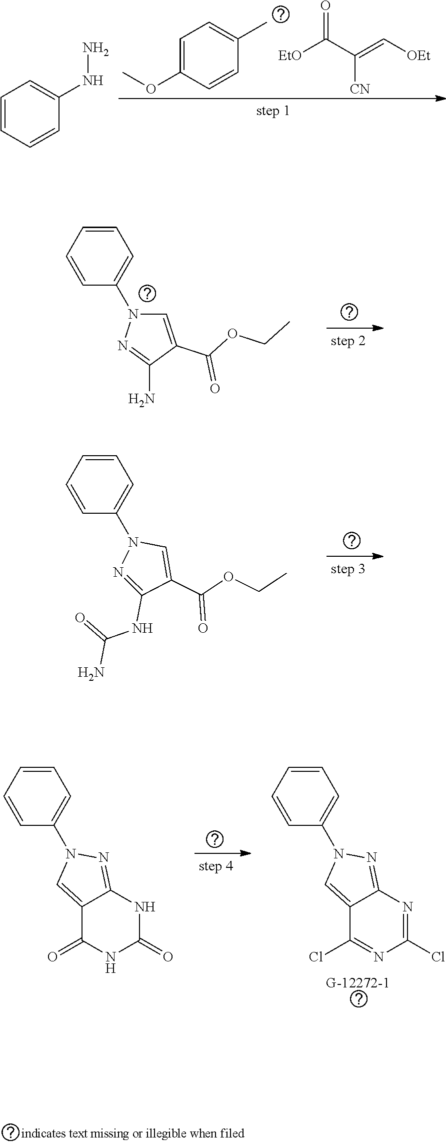

### Step 1, preparation of ethyl 3-amino-1-phenyl-1H-pyrazole-4-carboxylate (Intermediate 74a)

Anisaldehyde (25.0 g, 183 mmol, 1.00 equiv) and phenylhydrazine (19.8 g, 183 mmol, 1 equiv) were refluxed in toluene (300 mL) employing a Dean-Stark trap to remove water. The mixture was stirred at 120° C. for 16 h. The mixture was cooled to room temperature and concentrated under reduced pressure. The residue was dissolved in xylene (80 mL), to the above mixture was added ethyl (2E)-2-cyano-3-ethoxyprop-2-enoate (31.1 g, 183 mmol, 1.00 equiv), the mixture was stirred at 140° C. for 16 h. The mixture was cooled to room temperature and concentrated under reduced pressure. The residue was purified by silica gel column chromatography, eluted with PE/EA (3:1) to afford the intermediate (12.5 g). The intermediate was dissolved on in EtOH (25 mL), to the above mixture was added conc. HCl (5.4 mL). The mixture was stirred at 80° C. for 2 h. The mixture was cooled to room temperature and concentrated under reduced pressure. The mixture was diluted with NaHCO₃ (sat., aq.) (500 mL) and extracted with EA (2×300 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:5) to afford the title compound. MS (m/z) 232.15 [M+H]+.

### Step 2, preparation of ethyl 1-phenyl-3-ureido-1H-pyrazole-4-carboxylate (Intermediate 74b)

A mixture of ethyl 3-amino-1-phenyl-1H-pyrazole-4-carboxylate (9.00 g, 38.9 mmol, 1.00 equiv) and potassium cyanate (6.31 g, 77.8 mmol, 2.00 equiv) in AcOH (40 mL) was stirred at 25° C. for 4 h. The mixture was diluted with NaHCO₃ (sat., aq.) (250 mL) and extracted with EA (2×200 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:5) to afford the title compound. MS (m/z) 275.15 [M+H]+.

### Step 3, Preparation of 2-phenyl-2,7-dihydro-4H-pyrazolo[3,4-d]pyrimidine-4,6(5H)-dione (Intermediate 74c)

A mixture of ethyl 1-phenyl-3-ureido-1H-pyrazole-4-carboxylate (5.90 g, 21.5 mmol, 1.00 equiv) and sodium ethoxide (2.93 g, 43.0 mmol, 2.00 equiv) in EtOH (60 mL) was stirred at 80° C. for 2 h. The mixture was cooled to room temperature and concentrated under reduced pressure. The residue was diluted with water (100 mL) and the mixture was acidified to pH 5 with HCl (2 M). The resulting mixture was extracted with EA (5×300 mL). The combined organic layers were washed with brine (500 mL), dried over anhydrous Na₂SO₄. After filtration, the filtrate was concentrated under reduced pressure to afford the title compound. MS (m/z) 229.10 [M+H]+.

### Step 4. 4,6-dichloro-2-phenyl-2H-pyrazolo[3,4-d]pyrimidine (Intermediate 74d)

A mixture of 2-phenyl-2,7-dihydro-4H-pyrazolo[3,4-d]pyrimidine-4,6(5H)-dione (3 g, 13.2 mmol, 1.00 equiv) in POCl₃ (20 mL) was stirred at 100° C. for 16 h. The mixture was cooled to room temperature and concentrated under reduced pressure. The residue was diluted with EA (30 mL) and quenched by the addition of ice water (300 mL). The resulting mixture was extracted with EA (2×150 mL). The combined organic layers were washed with brine (200 mL), dried over anhydrous Na₂SO₄. After filtration, the filtrate was concentrated under reduced pressure. The residue was purified by silica gel column chromatography, eluted with DCM/EA (10:1) to afford the title compound. MS (m/z) 265.00, 267.00 [M+H]+. ¹H NMR (400 MHz, Chloroform-d) δ 8.64 (s, 1H), 7.98-7.90 (m, 2H), 7.64-7.49 (m, 3H).

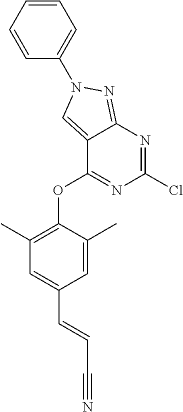 [SMILES: Cc1cc(/C=C/C#N)cc(C)c1Oc1nc(Cl)nc2nn(-c3ccccc3)cc12]

(E)-3-(4-((6-chloro-2-phenyl-2H-pyrazolo[3,4-d]pyrimidin-4-yl)oxy)-3,5-dimethylphenyl)acrylonitrile (Intermediate 74e) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 4,6-dichloro-2-phenyl-2H-pyrazolo[3,4-d]pyrimidine was used in place of 2,6-dichloro-N-[(2,4-dimethoxyphenyl)methyl]-5-nitro-pyrimidin-4-amine. MS (m/z): 402.0 [M+H]+.

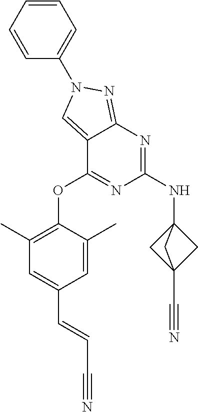 [SMILES: Cc1cc(/C=C/C#N)cc(C)c1Oc1nc(NC23CC(C#N)(C2)C3)nc2nn(-c3ccccc3)cc12]

(E)-3-((4-(4-(2-cyanovinyl)-2,6-dimethylphenoxy)-2-phenyl-2H-pyrazolo[3,4-d]pyrimidin-6-yl)amino)bicyclo[1.1.1]pentane-1-carbonitrile—Compound 74 was prepared using the procedure described for the synthesis of Compound 4 with the following modifications: (E)-3-(4-((6-chloro-2-phenyl-2H-pyrazolo[3,4-d]pyrimidin-4-yl)oxy)-3,5-dimethylphenyl)acrylonitrile was used in place of Intermediate 4a. MS (m/z) 474.1 [M+H]+. ¹H NMR (400 MHz, DMSO-d6) δ 9.39 (s, 1H), 8.31-7.78 (m, 3H), 7.71-7.48 (m, 5H), 7.45 (t, J=7.4 Hz, 1H), 6.45 (d, J=16.7 Hz, 1H), 2.26-1.80 (m, 12H).

### Example 75

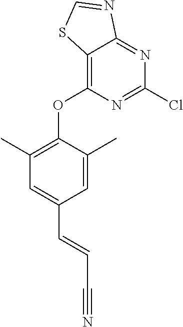 [SMILES: Cc1cc(/C=C/C#N)cc(C)c1Oc1nc(Cl)nc2ncsc12]

(E)-3-(4-((5-chlorothiazolo[4,5-d]pyrimidin-7-yl)oxy)-3,5-dimethylphenyl)acrylonitrile—Intermediate 75a was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 5,7-dichlorothiazolo[4,5-d]pyrimidine was used in place of 2,6-dichloro-N-[(2,4-dimethoxyphenyl)methyl]-5-nitro-pyrimidin-4-amine. MS (m/z) 343.0 [M+H]+.

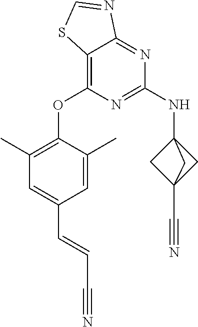 [SMILES: Cc1cc(/C=C/C#N)cc(C)c1Oc1nc(NC23CC(C#N)(C2)C3)nc2ncsc12]

(E)-3-((7-(4-(2-cyanovinyl)-2,6-dimethylphenoxy)thiazolo[4,5-d]pyrimidin-5-yl)amino)bicyclo[1.1.1]pentane-1-carbonitrile—Compound 75: Intermediate 75a (35.0 mg, 0.102 mmol) and 3-aminobicyclo[1.1.1]pentane-1-carbonitrile (55.2 mg, 0.510 mmol) were added to a vial and dissolved in NMP (0.20 mL). The vial was evacuated and backfilled once with argon gas, then the solution was heated to 140° C. for hour via microwave irradiation. The solution was cooled to room temperature then subjected to preparative HPLC (0-100% MeCN/water) to afford the title compound. MS (m/z) 415.15 [M+H]+. ¹H NMR (400 MHz, DMSO-d6) δ 9.71 (s, 1H), 8.33 (br s, 1H) 7.64 (d, J=16.7 Hz, 1H), 7.56 (s, 2H), 6.46 (d, J=16.7 Hz, 1H), 2.08 (s, 6H) 2.03 (br s, 6H).

### Example 76

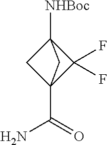 [SMILES: CC(C)(C)OC(=O)NC12CC(C(N)=O)(C1)C2(F)F]

tert-butyl (3-carbamoyl-2,2-difluorobicyclo[11.1.1]pentan-1-yl)carbamate (Intermediate 76a): 3-((tert-butoxycarbonyl)amino)-2,2-difluorobicyclo[1.1.1]pentane-1-carboxylic acid (100 mg, 0.380 mmol) was suspended in THE (3.8 mL) and cooled to 0° C. To the mixture was added triethylamine (34 μL, 0.46 mmol) and ethyl chloroformate (40 μL, 0.42 mmol). After one hour of stirring at 0° C., 30% aqueous NH₄OH (2.2 mL, 19 mmol) was added to the mixture which was stirred at room temperature for 2 hours. To the mixture was added water (30 mL) and the solution was extracted with EtOAc (3×20 mL). The combined organic layers were dried over Na₂SO₄ and concentrated in vacuo to afford the title compound. ¹H NMR (400 MHz, DMSO-d₆) δ 8.01 (br s, 1H), 7.47 (s, 1H), 7.31 (s, 1H), 2.31 (s, 2H), 1.94 (t, J=10.5 Hz, 2H), 1.39 (s, 9H). ¹⁹F NMR (377 MHz, DMSO-d₆) δ −121.76 (t, J=10.5 Hz).

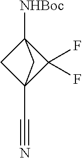 [SMILES: CC(C)(C)OC(=O)NC12CC(C#N)(C1)C2(F)F]

tert-butyl (3-cyano-2,2-difluorobicyclo[1.1.1]pentan-1-yl)carbamate (Intermediate 76b): Intermediate 76a (99.7 mg, 0.380 mmol) was suspended in dichloromethane (2 mL) and triethylamine (0.32 mL, 2.3 mmol). To mixture was cooled to 0° C. and trichloroacetyl chloride (0.13 mL. 1.2 mmol) was added to the mixture. After 1.5 hours stirring at 0° C., the mixture was diluted with dichloromethane (10 mL) and extracted with EtOAc (3×5 mL). The combined organic layers were dried over Na₂SO₄ and concentrated in vacuo. The crude material was subjected to silica gel flash column chromatography (0-100% EtOAc/hexanes) to afford a crude solid. This solid was used as is in the next transformation. ¹H NMR (400 MHz, Chloroform-d) 3.38 (dq, J=28.3, 7.2 Hz, 2H), 2.34 (s, 2H), 1.45 (s, 9H). ¹⁹F NMR (377 MHz, Chloroform-d) δ−120.55 (s).

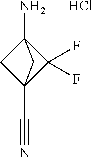

3-amino-2,2-difluorobicyclo[11.1.1]pentane-1-carbonitrile hydrochloride: Intermediate 76b (92.8 mg, 0.38 mmol) was suspended in a solution of 4.0 M HCl in 1,4-dioxane (2 mL, 8 mmol) and the mixture was stirred at room temperature for an hour. Trifluoroacetic acid (3 mL) was added. After 30 minutes, additional 4.0 M HCl in 1,4-dioxane (1 mL, 4 mmol) was added. The mixture was allowed to stir at room temperature overnight and was then concentrated in vacuo. The resulting solid was resuspended in trifluoroacetic acid (1 mL) and diethyl ether (5 mL) and hexanes (5 mL) was added to the mixture which was allowed to briefly stir at room temperature. The mixture was filtered to afford the HCl salt. ¹H NMR (400 MHz, DMSO-d₆) δ 8.87 (br s, 3H), 2.32 (s, 2H), 2.15-2.04 (m, 2H). ¹⁹F NMR (376 MHz, DMSO-d₆) δ −747.40 (s, 3F) −122.73 (t, J=10.4 Hz, 2F).

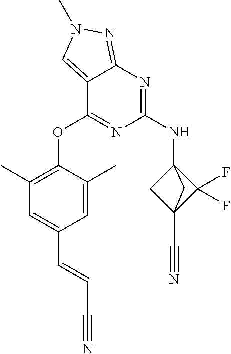 [SMILES: Cc1cc(/C=C/C#N)cc(C)c1Oc1nc(NC23CC(C#N)(C2)C3(F)F)nc2nn(C)cc12]

(E)-3-((4-(4-(2-cyanovinyl)-2,6-dimethylphenoxy)-2-methyl-2H-pyrazolo[3,4-d]pyrimidin-6-yl)amino)-2,2-difluorobicyclo[1.1.1]pentane-1-carbonitrile—Compound 76 Int-Compound 121A (30.0 mg, 0.883 mmol), 3-amino-2,2-difluoro-bicyclo[1.1.1]pentane-1-carbonitrile; hydrochloride (31.9 mg, 0.177 mmol), cesium carbonate (86.3 mg, 0.265 mmol), and rac BINAP Pd G3 precatalyst (8.8 mg, 0.0088 mmol) were combined in a vial. To the vial was added 1,4-dioxane (0.25 mL) and argon gas was bubbled through the mixture for 5 minutes. The vial was then sealed and heated to 120° C. for 3 hours. The mixture was then cooled to room temperature at which point 3-amino-2,2-difluoro-bicyclo[1.1.1]pentane-1-carbonitrile; hydrochloride (10.0 mg, 0.0555 mmol) and rac BINAP Pd G3 precatalyst (3.0 mg, 0.0030 mmol) was added to the mixture. Argon gas was bubbled through the mixture for 5 minutes and the reaction was sealed and heated at 120° C. for 2 additional hours. The mixture was cooled to room temperature and filtered over celite then subjected to preparative HPLC (0-100% MeCN/water) to afford the title compound (5.6 mg, 14.2% yield). MS (m/z) 448.13 [M+H]. ¹H NMR (400 MHz, DMSO-d6) δ 8.52 (s, 1H), 8.27 (s, 1H), 7.62 (d, J=16.7 Hz, 1H), 7.52 (s, 2H), 6.44 (d, J=16.7 Hz, 1H), 4.04 (s, 3H), 2.21 (br s, 4H) 2.07 (s, 6H). ¹⁹F NMR (377 MHz, DMSO) δ −118.90.

### Example 77

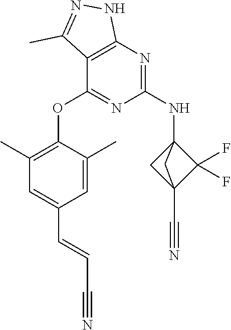 [SMILES: Cc1cc(/C=C/C#N)cc(C)c1Oc1nc(NC23CC(C#N)(C2)C3(F)F)nc2[nH]nc(C)c12]

(E)-3-((4-(4-(2-cyanovinyl)-2,6-dimethylphenoxy)-3-methyl-1H-pyrazolo[3,4-d]pyrimidin-6-yl)amino)-2,2-difluorobicyclo[1.1.1]pentane-1-carbonitrile—Compound 77: Prepared in a similar manner to Compound 76, except using Int-Compound 119A (30.0 mg, 0.883 mmol) instead of Int-Compound 120. MS (m/z) 448.16 [M+H]. ¹H NMR (400 MHz, DMSO-d6) δ 12.91 (s, 1H), 8.43 (s, 1H), 7.63 (d, J=16.8 Hz, 1H), 7.52 (s, 2H), 6.44 (d, J=16.6 Hz, 1H), 2.52 (s, 3H), 2.08 (s, 6H). 2.10 (br s, 4H). ¹⁹F NMR (377 MHz, DMSO) δ −118.79.

### Example 78

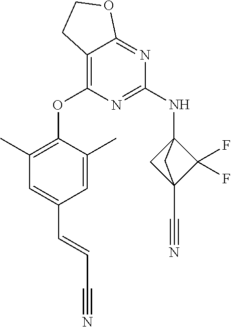 [SMILES: Cc1cc(/C=C/C#N)cc(C)c1Oc1nc(NC23CC(C#N)(C2)C3(F)F)nc2c1CCO2]

(E)-3-((4-(4-(2-cyanovinyl)-2,6-dimethylphenoxy)-5,6-dihydrofuro[2,3-d]pyrimidin-2-yl)amino)-2,2-difluorobicyclo[1.1.1]pentane-1-carbonitrile—Compound 78: Intermediate 118a (50 mg, 0.153 mmol), 3-amino-2,2-difluoro-bicyclo[1.1.1]pentane-1-carboxamide; hydrochloride (45.4 mg, 0.229 mmol), and potassium carbonate (105 mg, 0.763 mmol) were added to a vial and suspended in NMP (0.3 mL). The vial was sealed and heated to 140° C. for 2.5 hours. The mixture was cooled to room temperature and water (20 mL), brine (5 mL), and EtOAc (10 mL) were added to the solution which was subsequently mixed. The organic layer was isolated and the aqueous layer was further extracted with EtOAc (2×5 mL). The combined organic layers were dried over sodium sulfate, filtered, then concentrated in vacuo. The resulting residue was dissolved in DCM (1.0 mL) then triethylamine (0.13 ml. 0.92 mmol) and trichloroacetyl chloride (0.052 uL, 0.459 mmol) were added in sequence. The solution was stirred at room temperature for 1 hour, at which time saturated aqueous sodium bicarbonate (2 mL) was added. The mixture was extracted with EtOAc (3×1 mL) and the combined organic layers were dried over sodium sulfate, filtered, then concentrated in vacuo. The resulting residue was subjected to preparative HPLC (0-100% MeCN/water) to afford the title compound. MS (m/z) 436.08 [M+H]+. ¹H NMR (400 MHz, Acetonitrile-d3) δ 7.46 (d, J=16.7 Hz, 1H), 7.34 (s, 2H), 6.38 (s, 1H), 6.04 (d, J=16.7 Hz, 1H), 4.68-4.61 (m, 2H), 3.12 (t, J=8.6 Hz, 2H), 2.13 (s, 6H), 2.10 (br s, 4H). ¹⁹F NMR (376 MHz, CD3CN) δ −120.91 (br s).

### Example 79

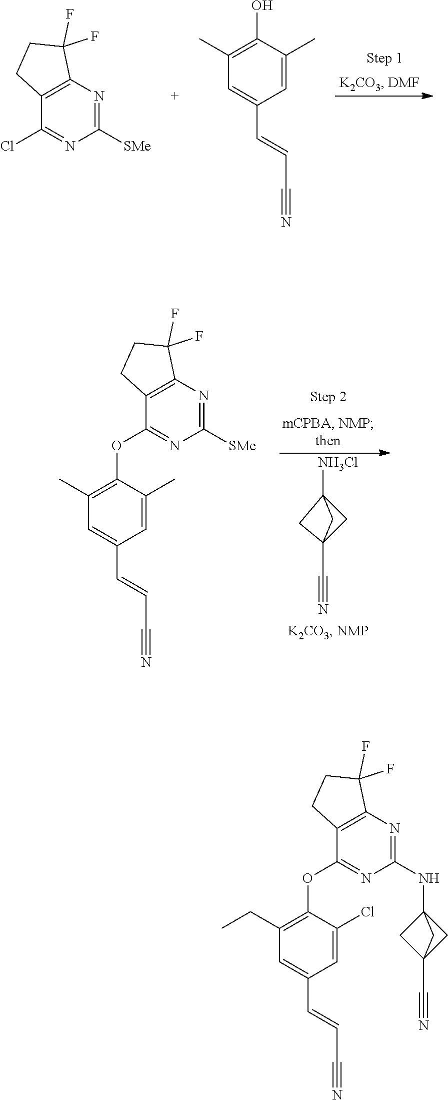

Step 1: (E)-3-(4-((7,7-difluoro-2-(methylthio)-6,7-dihydro-5H-cyclopenta[d]pyrimidin-4-yl)oxy)-3,5-dimethylphenyl)acrylonitrile (Intermediate 79a): (E)-3-(4-hydroxy-3,5-dimethyl-phenyl)prop-2-enenitrile (132 mg, 0.761 mmol), 4-chloro-7,7-difluoro-2-(methylthio)-6,7-dihydro-5H-cyclopenta[d]pyrimidine (150 mg, 0.634 mmol), and potassium carbonate (175 mg, 1.27 mmol) were suspended in DMF (2.4 mL) and the solution was stirred at room temperature for 2.5 hours. To the solution was added water (5 mL) and the resulting solids were filtered off and lyophilized to afford the crude title compound which was used as is in the subsequent transformation.

Step 2: (E)-3-((4-(4-(2-cyanovinyl)-2,6-dimethylphenoxy)-7,7-difluoro-6,7-dihydro-5H-cyclopenta[d]pyrimidin-2-yl)amino)bicyclo[1.1.1]pentane-1-carbonitrile—Compound 79: mCBPA (64.7 mg, 0.281 mmol) was added to a solution of Intermediate 79a (50.0 mg, 0.134 mmol) in NMP (0.30 mL) and the mixture was stirred at room temperature for two hours. To the mixture was added potassium carbonate (92.6 mg, 0.670 mmol) and 3-aminobicyclo[1.1.1]pentane-1-carbonitrile; hydrochloride (48.4 mg, 0.335 mmol). The mixture was stirred at 40° C. for 30 minutes, then additional NMP (0.15 mL) was added and the solution was stirred at 50° C. for 1.5 hours. Additional potassium carbonate (100 mg, 0.362 mmol) was added and the solution was stirred at 50° C. overnight. The mixture was then cooled to room temperature and water (2 mL) was added. The mixture was extracted with EtOAc (3×1 mL) and the combined organic layers were concentrated in vacuo and subjected to preparative HPLC (0-100% MeCN/water) and lyophilized to afford the title compound. MS (m/z) 434.23 [M+H]+. ¹H NMR (400 MHz, DMSO-d6) δ 8.47 (s, 1H), 7.62 (d, J=16.7 Hz, 1H), 7.55 (s, 2H), 6.43 (d, J=16.7 Hz, 1H), 2.94 (br s, 2H), 2.63 (m, 2H), 2.07 (s, 6H), 1.96 (br s, 6H). ¹⁹F NMR (377 MHz, DMSO) δ −93.93.

### Example 80

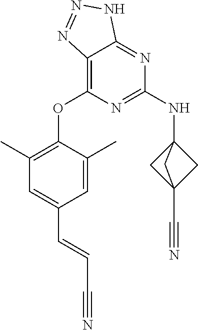 [SMILES: Cc1cc(/C=C/C#N)cc(C)c1Oc1nc(NC23CC(C#N)(C2)C3)nc2[nH]nnc12]

€-3-((7-(4-(2-cyanovinyl)-2,6-dimethylphenoxy)-3H-[1,2,3]triazolo[4,5-d]pyrimidin-5-yl)amino)bicyclo[1.1.1]pentane-1-carbonitrile—Compound 80: (E)-3-((4,5-diamino-6-(4-(2-cyanovinyl)-2,6-dimethylphenoxy)pyrimidin-2-yl)amino)bicyclo[1.1.1]pentane-1-carbonitrile (20.0 mg, 0.0516 mmol) was suspended in water (0.1 mL) and acetic acid (0.2 mL) then cooled to 0° C. To the solution was added an 0.9 M aqueous solution of sodium nitrite (0.1 mL, 0.09 mmol). After 15 minutes, the solution was extracted with ethyl acetate (1 mL). The organic layer was washed with saturated sodium bicarbonate (3×1 mL). The combined aqueous layers were further extracted with ethyl acetate (2×1 mL). The organic layers were combined and concentrated in vacuo. The crude residue was subjected to preparative HPLC (15-95% MeCN/H₂O) and lyophilized to provide the title compound. MS (m/z) 399.14 [M+H]+. ¹H NMR (400 MHz, DMSO-d6) δ 15.79 (br s, 1H), 8.34 (br s, 1H), 7.64 (d, J=16.7 Hz, 1H), 7.57 (s, 2H), 6.46 (d, J=16.7 Hz, 1H), 2.11 (s, 6H), 2.10 (br s, 6H).

### Example 81

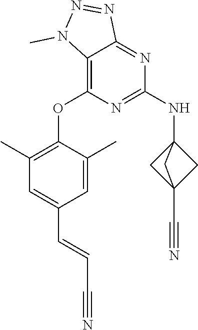 [SMILES: Cc1cc(/C=C/C#N)cc(C)c1Oc1nc(NC23CC(C#N)(C2)C3)nc2nnn(C)c12]

(E)-3-((7-(4-(2-cyanovinyl)-2,6-dimethylphenoxy)-1-methyl-11H-[1,2,3]triazolo[4,5-d]pyrimidin-5-yl)amino)bicyclo[1.1.1]pentane-1-carbonitrile—Compound 81: Compound 80 (40.0 mg, 0.100 mmol) and potassium carbonate (27.8 mg, 0.201 mmol) were charged to a vial and suspended in DMF (1.0 mL). To the solution was added iodomethane (7.5 uL, 0.120 mmol) and the mixture was stirred at room temperature. After one hour, water (1 ml) was added the mixture and the solution was extracted with ethyl acetate (3×1 mL). The organic layers were combined and concentrated in vacuo. The crude residue was subjected to subjected to preparative HPLC (15-95% MeCN/H₂O) and lyophilized to provide the title compound. MS (m/z) 413.14 [M+H]+. ¹H NMR (400 MHz, DMSO-d6) δ 8.19 (s, 1H), 7.64 (d, J=16.7 Hz, 1H), 7.56 (s, 2H), 6.46 (d, J=16.7 Hz, 1H), 4.43 (s, 3H), 2.13 (s, 6H), 2.10 (br s, 6H).

### Example 82

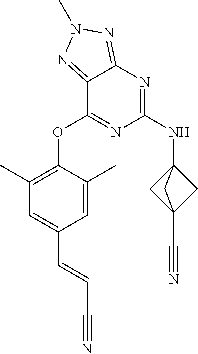 [SMILES: Cc1cc(/C=C/C#N)cc(C)c1Oc1nc(NC23CC(C#N)(C2)C3)nc2nn(C)nc12]

(E)-3-((7-(4-(2-cyanovinyl)-2,6-dimethylphenoxy)-2-methyl-2H-[1,2,3]triazolo[4,5-d]pyrimidin-5-yl)amino)bicyclo[1.1.1]pentane-1-carbonitrile—Compound 82: Compound 80 (40.0 mg, 0.100 mmol) and potassium carbonate (27.8 mg, 0.201 mmol) were charged to a vial and suspended in DMF (1.0 mL). To the solution was added iodomethane (7.5 uL, 0.120 mmol) and the mixture was stirred at room temperature. After one hour, water (1 ml) was added the mixture and the solution was extracted with ethyl acetate (3×1 mL). The organic layers were combined and concentrated in vacuo. The crude residue was subjected to subjected to preparative HPLC (15-95% MeCN/H₂O) and lyophilized to provide the title compound. MS (m/z) 413.17 [M+H]+. ¹H NMR (400 MHz, DMSO-d6) δ 8.29 (s, 1H), 7.70-7.51 (m, 3H), 6.45 (d, J=16.7 Hz, 1H), 4.41 (s, 3H), 2.10 (s, 6H), 1.97 (br ss, 6H).

### Example 83

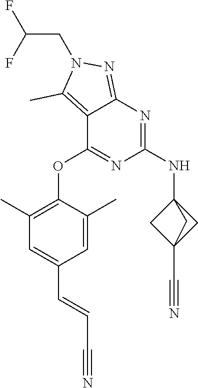 [SMILES: Cc1cc(/C=C/C#N)cc(C)c1Oc1nc(NC23CC(C#N)(C2)C3)nc2nn(CC(F)F)c(C)c12]

(E)-3-((4-(4-(2-cyanovinyl)-2,6-dimethylphenoxy)-2-(2,2-difluoroethyl)-3-methyl-2H-pyrazolo[3,4-d]pyrimidin-6-yl)amino)bicyclo[11.1.1]pentane-1-carbonitrile—Compound 83: A vial was charged with the trifluoroacetic acid salt of Compound 119 (30.0 mg, 0.0571 mmol) and cesium carbonate (93.0 mg, 0.285 mmol). To the vial was added NMP (0.5 mL) and two drops of 1,1-difluoro-2-iodo-ethane. The mixture was stirred at room temperature for 3 hours, at which point the mixture was filtered over celite and subjected to preparative HPLC (15-95% MeCN/H₂O) and lyophilized to afford the title compound. MS (m/z) 476.26 [M+H]+. ¹H NMR (400 MHz, DMSO-d6) δ 8.44 (br s, 1H), 7.57 (m, 3H), 6.60-6.20 (m, 2H), 4.56 (s, 2H), 2.54 (s, 3H), 2.10 (s, 6H), 1.96 (br s, 6H). ¹⁹F NMR (377 MHz, DMSO-d6) δ −122.50 (dt, J=54.4, 15.0 Hz).

### Example 84

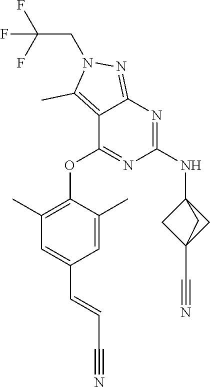 [SMILES: Cc1cc(/C=C/C#N)cc(C)c1Oc1nc(NC23CC(C#N)(C2)C3)nc2nn(CC(F)(F)F)c(C)c12]

(E)-3-((4-(4-(2-cyanovinyl)-2,6-dimethylphenoxy)-3-methyl-2-(2,2,2-trifluoroethyl)-2H-pyrazolo[3,4-d]pyrimidin-6-yl)amino)bicyclo[1.1.1]pentane-1-carbonitrile—Compound 84: A vial was charged with the trifluoroacetic acid salt of Compound 119 (30.0 mg, 0.0571 mmol) and cesium carbonate (93.0 mg, 0.285 mmol). To the vial was added NMP (0.5 mL) and two drops of 2,2,2-trifluoroethyl trifluoromethanesulfonate. The mixture was stirred at room temperature for 3 hours, at which point another portion of 2,2,2-trifluoroethyl trifluoromethanesulfonate was added. After an additional 1.5 hours, the mixture was filtered over celite and subjected to preparative HPLC (15-95% MeCN/H₂O) and lyophilized to afford the title compound. MS (m/z) 494.28 [M+H]+. H NMR (400 MHz, DMSO-d6) δ 8.44 (br s, 1H), 7.54 (m, 3H), 6.44 (d, J=16.7 Hz, 1H), 5.04 (d, J=54.4 Hz, 2H), 2.54 (s, 3H), 2.11 (s, 6H), 1.96 (br s, 6H). ¹⁹F NMR (377 MHz, DMSO) δ −69.88.

### Example 85

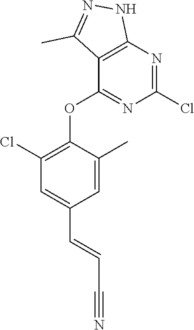 [SMILES: Cc1cc(/C=C/C#N)cc(Cl)c1Oc1nc(Cl)nc2[nH]nc(C)c12]

(E)-3-(3-chloro-4-((6-chloro-3-methyl-1H-pyrazolo[3,4-d]pyrimidin-4-yl)oxy)-5-methylphenyl)acrylonitrile (Intermediate 85a): (E)-3-(3-chloro-4-hydroxy-5-methylphenyl)acrylonitrile (250 mg, 0.739 mmol), 4,6-dichloro-3-methyl-1H-pyrazolo[3,4-d]pyrimidine (150 mg, 0.739 mmol), and potassium carbonate (306 mg, 2.22 mmol) were suspended in NMP (1.5 mL) and the solution was stirred at room temperature for 1 hour. To the solution was added water (5 mL) and the mixture was extracted with ethyl acetate (3×1 mL). The combined organic layers were dried over Na₂SO₄ and concentrated in vacuo onto silica gel and subjected to silica gel flash column chromatography (0-100% EtOAc/hexanes) to afford the title product. ¹H NMR (400 MHz, Acetonitrile-d3) δ 11.57 (br s, 1H), 7.65 (d, J=2.1 Hz, 1H), 7.54 (d, J=2.0 Hz, 1H), 7.50 (d, J=16.7 Hz, 1H), 6.17 (d, J=16.7 Hz, 1H), 2.70 (s, 3H), 2.25 (d, J=0.8 Hz, 3H). MS (m/z) 360.10 [M+H]+.

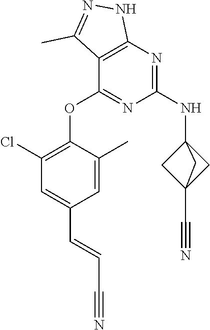 [SMILES: Cc1cc(/C=C/C#N)cc(Cl)c1Oc1nc(NC23CC(C#N)(C2)C3)nc2[nH]nc(C)c12]

(E)-3-((4-(2-chloro-4-(2-cyanovinyl)-6-methylphenoxy)-3-methyl-1H-pyrazolo[3,4-d]pyrimidin-6-yl)amino)bicyclo[1.1.1]pentane-1-carbonitrile—Compound 85: A vial was charged with Intermediate 85a (58.0 mg, 0.161 mmol), 3-aminobicyclo[1.1.1]pentane-1-carbonitrile; hydrochloride (163 mg, 1.13 mmol), and potassium carbonate (223 mg, 1.61 mmol) followed by NMP (0.3 mmol). The solution was heated at 140° C. for 1.5 hours, then at 150° C. for two hours. The mixture was cooled to room temperature at which point water (1.0 mL) was added. The solution was extracted with EtOAc (3×1 mL) and the combined organic layers were dried over sodium sulfate, filtered, and concentrated in vacuo. The resulting residue was subjected to preparative HPLC (0-100% MeCN/H₂O, 0.1% trifluoroacetic acid modifier). The fractions with the product were pooled and diluted with EtOAc (20 mL) and washed with saturated aqueous sodium bicarbonate (10 mL). The aqueous layer was extracted with EtOAc (2×20 mL) and the combined organic layers were dried over sodium sulfate, filtered, and concentrated in vacuo. The resulting residue was subjected to flash column chromatography (0-20% MeOH/DCM) to afford the title compound. MS (m/z) 432.10 [M+H]+. ¹H NMR (400 MHz, Acetonitrile-d3) δ 10.69 (br s, 1H), 7.66 (d, J=2.1 Hz, 1H), 7.54 (s, 1H), 7.51 (d, J=16.7 Hz, 1H), 6.30 (br s, 1H), 6.17 (d, J=16.7 Hz, 1H), 2.58 (s, 3H), 2.23 (s, 3H), 2.18 (br s, 6H).

### Example 86

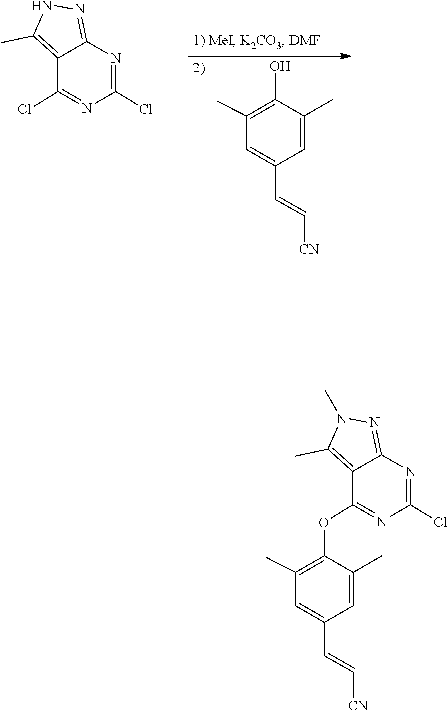

Preparation of €-3-(4-((6-chloro-2,3-dimethyl-2H-pyrazolo[3,4-d]pyrimidin-4-yl)oxy)-3,5-dimethylphenyl)acrylonitrile (Intermediate 86a): To a solution of 4,6-dichloro-3-methyl-1H-pyrazolo[3,4-d]pyrimidine (170 mg, 0.84 mmol) in DMF was added potassium carbonate (579 mg, 4.19 mmol) and iodomethane (155 mg, 1.1 mmol). The mixture was stirred at room temperature for 1 hour. Then to the mixture was added €-3-(4-hydroxy-3,5-dimethyl-phenyl)prop-2-enenitrile (144 mg, 0.84 mmol). The mixture was stirred at room temperature overnight. Water was added to the mixture and the solution was extracted with EtOAc. The organic phase was dried over MgSO₄, filtered and concentrated in vacuo. The resulting residue was subjected to silica gel flash column chromatography, (0-100% hexane/EtOAc) to afford the titled product. MS (m/z) 354.08. ¹H NMR (400 MHz, Acetonitrile-d3) δ 7.48 (d, J=16.7 Hz, 1H), 7.39 (s, 2H), 6.09 (d, J=16.7 Hz, 1H), 4.07 (s, 3H), 2.81 (s, 3H), 2.17 (s, 6H).

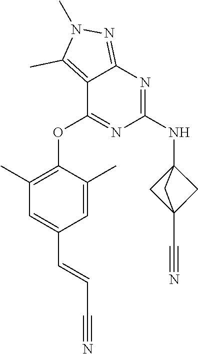 [SMILES: Cc1cc(/C=C/C#N)cc(C)c1Oc1nc(NC23CC(C#N)(C2)C3)nc2nn(C)c(C)c12]

Preparation of (E)-3-((4-(4-(2-cyanovinyl)-2,6-dimethylphenoxy)-2,3-dimethyl-2H-pyrazolo[3,4-d]pyrimidin-6-yl)amino)bicyclo[1.1.1]pentane-1-carbonitrile—Compound 86: the title compound was prepared using the procedure described for the synthesis of Compound 4 with the modification that Intermediate 86a was used in place of Intermediate 4a. MS (m/z) 426.21. ¹H NMR (400 MHz, Methanol-d4) δ 7.59-7.47 (m, 3H), 6.25 (d, J=16.7 Hz, 1H), 3.99 (s, 3H), 2.79 (s, 3H), 2.25-2.11 (m, 12H).

### Example 87

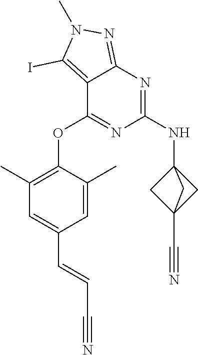 [SMILES: Cc1cc(/C=C/C#N)cc(C)c1Oc1nc(NC23CC(C#N)(C2)C3)nc2nn(C)c(I)c12]

(E)-3-((4-(4-(2-cyanovinyl)-2,6-dimethylphenoxy)-3-iodo-2-methyl-2H-pyrazolo[3,4-d]pyrimidin-6-yl)amino)bicyclo[1.1.1]pentane-1-carbonitrile—Compound 87 was prepared using the two step procedure described for the synthesis of Compound 86 with the modification that 4,6-dichloro-3-iodo-1H-pyrazolo[3,4-d]pyrimidine was used in place of 4,6-dichloro-3-methyl-1H-pyrazolo[3,4-d]pyrimidine. MS (m/z) 538.20. ¹H NMR (400 MHz, Methanol-d4) δ 7.54 (d, J=16.7 Hz, 1H), 7.48 (s, 2H), 6.24 (d, J=16.7 Hz, 1H), 4.13 (s, 3H), 2.25-2.16 (m, 12H).

### Example 88

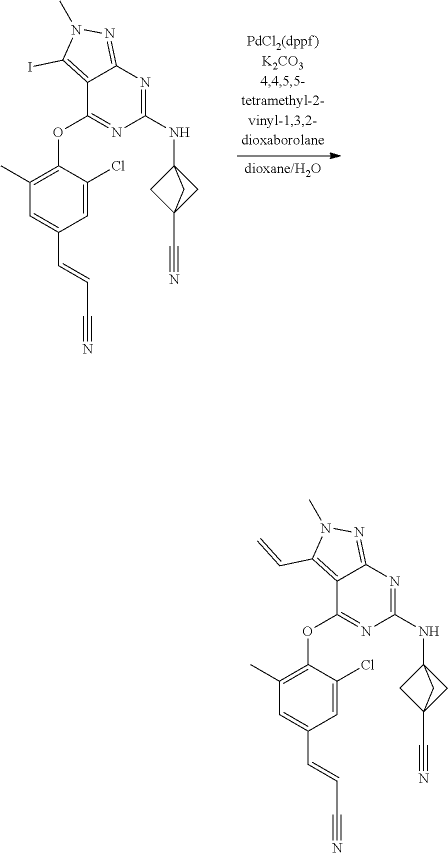 [SMILES: Cc1cc(/C=C/C#N)cc(Cl)c1Oc1nc(NC23CC(C#N)(C2)C3)nc2nn(C)c(I)c12 ; C=Cc1c2c(Oc3c(C)cc(/C=C/C#N)cc3Cl)nc(NC34CC(C#N)(C3)C4)nc2nn1C]

Preparation of (E)-3-((4-(4-(2-cyanovinyl)-2,6-dimethylphenoxy)-2-methyl-3-vinyl-2H-pyrazolo[3,4-d]pyrimidin-6-yl)amino)bicyclo[1.1.1]pentane-1-carbonitrile—Compound 88: A mixture of 3-[[4-[4-[(E)-2-cyanovinyl]-2,6-dimethyl-phenoxy]-3-iodo-2-methyl-pyrazolo[3,4-d]pyrimidin-6-yl]amino]bicyclo[1.1.1]pentane-1-carbonitrile (20 mg, 0.037 mmol), 4,4,5,5-tetramethyl-2-vinyl-1,3,2-dioxaborolane (11.5 mg, 0.0744 mmol), potassium carbonate (25.7 mg, 0.186 mmol), PdCl₂(dppf) (4 mg, 0.0056 mmol) in 9:1 dioxane/water (2 mL) was stirred at 90° C. overnight. To mixture was added saturated aqueous NaHCO₃ and the solution was extracted with EtOAc. The organic phase was separated, dried over MgSO4, filtered, and concentrated in vacuo. The resulting residue was subjected to reverse phase HPLC (5-100% acetonitrile/water, modified with 0.1% TFA) and lyophilized. The resulting solid was subjected to silica gel flash column chromatography (0-100% hexane/EtOAc) to afford the title compound. MS (m/z) 438.27 [M+H]+. ¹H NMR (400 MHz, Methanol-d4) δ 7.60-7.46 (m, 3H), 7.10 (dd, J=17.4, 11.8 Hz, 1H), 6.52 (d, J=17.4 Hz, 1H), 6.25 (d, J=16.7 Hz, 1H), 5.94 (d, J=11.8 Hz, 1H), 4.10 (s, 3H), 2.22-2.15 (m, 12H).

### Example 89

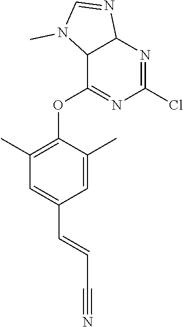 [SMILES: Cc1cc(/C=C/C#N)cc(C)c1OC1=NC(Cl)=NC2N=CN(C)C12]

(E)-3-(4-((2-chloro-7-methyl-4,5-dihydro-7H-purin-6-yl)oxy)-3,5-dimethylphenyl)acrylonitrile (Intermediate 89a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the modification that 2,6-dichloro-7-methyl-4,5-dihydro-7H-purine was used in place of 2,6-dichloro-N-[(2,4-dimethoxyphenyl)methyl]-5-nitro-pyrimidin-4-amine.

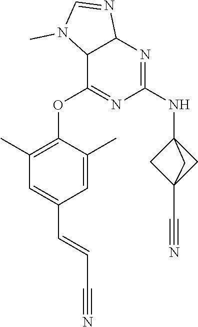 [SMILES: Cc1cc(/C=C/C#N)cc(C)c1OC1=NC(NC23CC(C#N)(C2)C3)=NC2N=CN(C)C12]

(E)-3-((6-(4-(2-cyanovinyl)-2,6-dimethylphenoxy)-7-methyl-4,5-dihydro-7H-purin-2-yl)amino)bicyclo[1.1.1]pentane-1-carbonitrile—Compound 89 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile with the modification that Intermediate 89a was used in place of E)-3-[4-[2-chloro-7-[(4-methoxyphenyl)methyl]-8-methyl-purin-6-yl]oxy-3,5-dimethyl-phenyl]prop-2-enenitrile. MS (m/z) 412.19 [M+H]+. ¹H NMR (400 MHz, Methanol-d4) δ 8.78 (s, 1H), 7.61-7.48 (m, 3H), 6.26 (d, J=16.6 Hz, 1H), 4.22 (s, 3H), 2.22-2.16 (m, 12H).

### Example 90

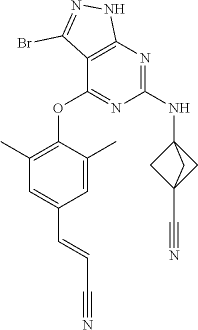 [SMILES: Cc1cc(/C=C/C#N)cc(C)c1Oc1nc(NC23CC(C#N)(C2)C3)nc2[nH]nc(Br)c12]

(E)-3-((3-bromo-4-(4-(2-cyanovinyl)-2,6-dimethylphenoxy)-1H-pyrazolo[3,4-d]pyrimidin-6-yl)amino)bicyclo[1.1.1]pentane-1-carbonitrile—Compound 90 was synthesized using the procedure described for the synthesis of Compound 10, with the modification that 3-bromo-4-chloro-6-methylsulfanyl-1H-pyrazolo[3,4-d]pyrimidine was used in place of 4-chloro-6-methylsulfanyl-1H-pyrazolo[3,4-d]pyrimidine. MS (m/z) 476.20, 478.15 [M+H]+. ¹H NMR (400 MHz, Methanol-d₄) δ 7.54 (d, J=16.7 Hz, 1H), 7.48 (s, 2H), 6.24 (d, J=16.7 Hz, 1H), 2.42 (d, J=56.4 Hz, 2H), 2.19 (s, 6H), 2.06 (d, J=21.6 Hz, 4H).

### Example 91

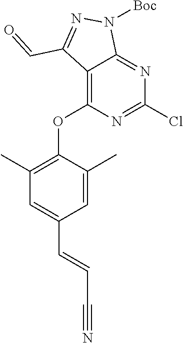 [SMILES: Cc1cc(/C=C/C#N)cc(C)c1Oc1nc(Cl)nc2c1c(C=O)nn2C(=O)OC(C)(C)C]

tert-butyl (E)-6-chloro-4-(4-(2-cyanovinyl)-2,6-dimethylphenoxy)-3-formyl-1H-pyrazolo[3,4-d]pyrimidine-1-carboxylate (Intermediate 91a) was synthesized using the procedure described for the synthesis of Intermediate 1f, with the modifications that 4,6-dichloro-1H-pyrazolo[3,4-d]pyrimidine-3-carbaldehyde was used in place of (E)-3-(3-chloro-5-ethyl-4-hydroxyphenyl)acrylonitrile, the first step reaction was heated at 50° C., and product was isolated using silica gel flash column chromatography before Boc protection(0-100% hexane/EtOAc)¹H NMR (400 MHz, Chloroform-d) δ 10.29 (s, 1H), 7.38 (d, J=16.6 Hz, 1H), 7.25 (s, 2H), 5.88 (d, J=16.6 Hz, 1H), 2.17 (s, 6H), 1.77 (s, 9H).

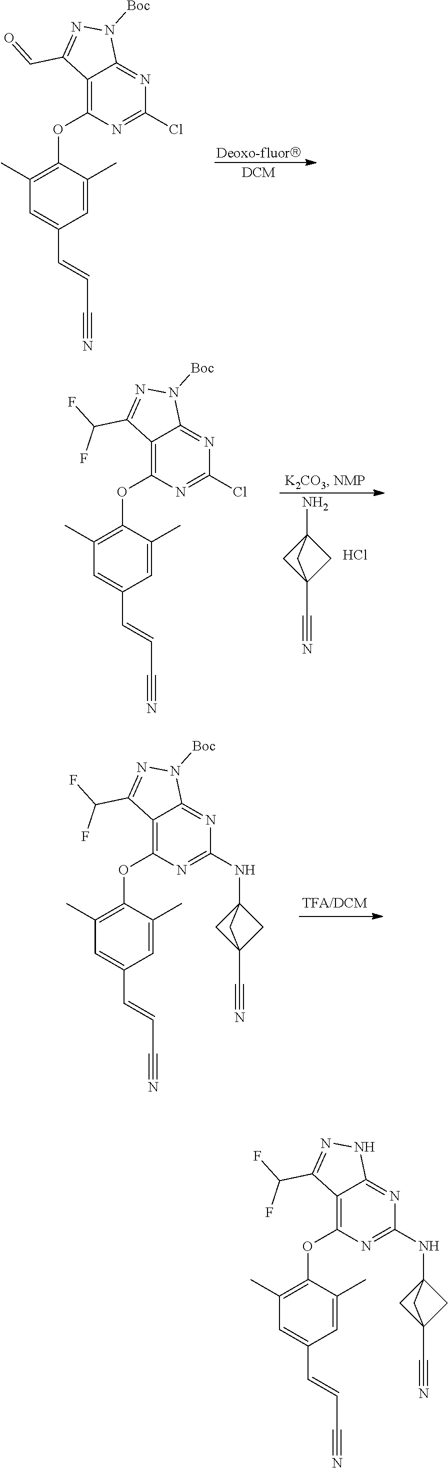

### (E)-3-((4-(4-(2-cyanovinyl)-2,6-dimethylphenoxy)-3-(difluoromethyl)-1H-pyrazolo[3,4-d]pyrimidin-6-yl)amino)bicyclo[1.1.1]pentane-1-carbonitrile (Intermediate 91b)

Step 1: preparation of tert-butyl (E)-6-chloro-4-(4-(2-cyanovinyl)-2,6-dimethylphenoxy)-3-(difluoromethyl)-1H-pyrazolo[3,4-d]pyrimidine-1-carboxylate: To a solution of Intermediate 91a (25 mg, 0.051 mmol) in DCM (2 mL) was added Deoxy-Fluor® (81 mg, 0.165 mmol). The mixture was stirred at room temperature for 5 hours. To the mixture was added saturated aqueous NaHCO₃ and the solution was extracted with DCM. The organic phase was dried over MgSO4, filtered, concentrated in vacuo and subjected to silica gel flash column chromatography (0-100% hexane/EtOAc) to afford the title compound. ¹H NMR (400 MHz, Chloroform-d) δ 7.39 (d, J=16.6 Hz, 1H), 7.26 (s, 2H), 6.97 (t, J=53.0 Hz, 1H), 5.89 (d, J=16.6 Hz, 1H), 2.17 (s, 6H), 1.75 (s, 9H). ¹⁹F NMR (377 MHz, Chloroform-d) δ −114.05, −114.19.

Step 2: preparation of tert-butyl (E)-6-((3-cyanobicyclo[1.1.1]pentan-1-yl)amino)-4-(4-(2-cyanovinyl)-2,6-dimethylphenoxy)-3-(difluoromethyl)-1H-pyrazolo[3,4-d]pyrimidine-1-carboxylate (Intermediate 91c): To a solution of Intermediate 91b (20 mg, 0.042 mmol) in NMP (2.5 mL) was added potassium carbonate (29 mg, 0.21 mmol) and 3-aminobicyclo[1.1.1]pentane-1-carbonitrile; hydrochloride (10.6 mg, 0.073 mmol). The mixture was heated at 60° C. overnight then cooled to room temperature. To the mixture was added saturated aqueous NaHCO₃ and the solution was extracted with EtOAc. The combined organic layers were dried with MgSO₄, filtered, concentrated in vacuo and subjected to silica gel flash column chromatography (0-100% hexane/EtOAc) to afford the title compound. MS (m/z) 547.65 [M+H]+.

Step 3: preparation of (E)-3-((4-(4-(2-cyanovinyl)-2,6-dimethylphenoxy)-3-(difluoromethyl)-1H-pyrazolo[3,4-d]pyrimidin-6-yl)amino)bicyclo[1.1.1]pentane-1-carbonitrile—Compound 93: A solution of Intermediate 91c (6 mg, 0.01 mmol) in 1:1 DCM/TFA (1 mL) was stirred at room temperature for 1 hour. The mixture was concentrated in vacuo and the resulting residue was subjected to reverse phase HPLC (5-100% MeCN/H₂O, containing 0.1% TFA) and lyophilized. The resulting solid was subjected to silica gel flash column chromatography (0-60% hexane/EtOAc). MS (m/z) 448.18 [M+H]+. MS (m/z) 448.18 [M+H]+. ¹H NMR (400 MHz, Methanol-d4) δ 7.54 (d, J=16.6 Hz, 1H), 7.48 (s, 2H), 6.95 (t, J=53.7 Hz, 1H), 6.23 (d, J=16.7 Hz, 1H), 2.56-2.00 (m, 12H). ¹⁹F NMR (376 MHz, Methanol-d₄) δ −114.34 (d, J=6.1 Hz), −114.47.

### Examples 92 and 93

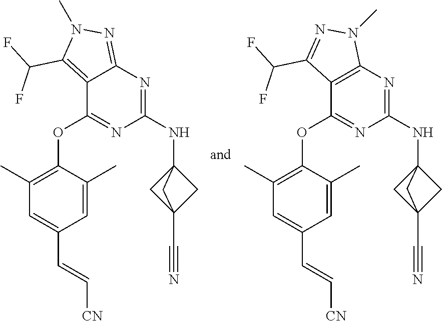 [SMILES: Cc1cc(/C=C/C#N)cc(C)c1Oc1nc(NC23CC(C#N)(C2)C3)nc2nn(C)c(C(F)F)c12 ; Cc1cc(/C=C/C#N)cc(C)c1Oc1nc(NC23CC(C#N)(C2)C3)nc2c1c(C(F)F)nn2C]

Preparation of (E)-3-((4-(4-(2-cyanovinyl)-2,6-dimethylphenoxy)-3-(difluoromethyl)-2-methyl-2H-pyrazolo[3,4-d]pyrimidin-6-yl)amino)bicyclo[1.1.1]pentane-1-carbonitrile (Compound 92) and (E)-3-((4-(4-(2-cyanovinyl)-2,6-dimethylphenoxy)-3-(difluoromethyl)-1-methyl-1H-pyrazolo[3,4-d]pyrimidin-6-yl)amino)bicyclo[1.1.1]pentane-1-carbonitrile (Compound 93): To a solution of Compound 91 (10 mg, 0.023 mmol) in THE (2 mL) was added K₂CO₃ (15.6 mg, 0.112 mmol) and iodomethane (4.7 mg, 0.034 mmol). The mixture was stirred at 55° C. for 3 days and was then cooled to room temperature and concentrated in vacuo. The resulting residue was subjected to reverse phase HPLC (5-100% MeCN/H2O). The major compound was Compound 93 and the minor compound was Compound 92. The compound eluted first was Compound 92 and the compound eluted second was Compound 93. For Compound 92: MS (m/z) 462.20 [M+H]+. ¹H NMR (400 MHz, Methanol-d4) δ 7.59 (d, J=25.5 Hz, 1H), 7.53-7.36 (m, 3H), 6.25 (d, J=16.7 Hz, 1H), 4.24 (t, J=1.1 Hz, 3H), 2.30-2.19 (m, 12H). For Compound 93: MS (m/z) 462.24 [M+H]+. ¹H NMR (400 MHz, Methanol-d4) δ 7.60-7.38 (m, 3H), 6.93 (t, J=53.8 Hz, 1H), 6.23 (d, J=16.6 Hz, 1H), 3.92 (s, 3H), 2.58-2.3 (m, 2H), 2.16-2.01 (m, 10H).

### Example 94

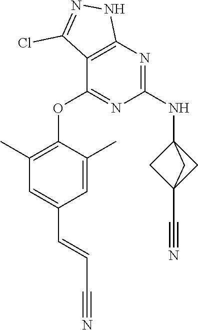 [SMILES: Cc1cc(/C=C/C#N)cc(C)c1Oc1nc(NC23CC(C#N)(C2)C3)nc2[nH]nc(Cl)c12]

(E)-3-((3-chloro-4-(4-(2-cyanovinyl)-2,6-dimethylphenoxy)-1H-pyrazolo[3,4-d]pyrimidin-6-yl)amino)bicyclo[1.1.1]pentane-1-carbonitrile—Compound 94 was prepared using the procedure described for the synthesis of Compound 1 with the modification that (E)-3-(4-hydroxy-3,5-dimethylphenyl)acrylonitrile was used in place of (E)-3-(3-ethyl-4-hydroxy-5-methylphenyl)acrylonitrile and 3,4,6-trichloro-3a,7a-dihydro-1H-pyrazolo[3,4-d]pyrimidine was used in place of 4,6-dichloro-3-methyl-3a,7a-dihydro-1H-pyrazolo[3,4-d]pyrimidine and the reaction was stirred at room temperature overnight. MS (m/z) 432.17 [M+H]+. ¹H NMR (400 MHz, Methanol-d4) δ 7.60-7.37 (m, 3H), 6.24 (d, J=16.6 Hz, 1H), 2.54 (bs, 2H), 2.21-2.09 (m, 10H).

### Example 95

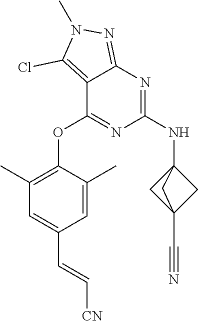 [SMILES: Cc1cc(/C=C/C#N)cc(C)c1Oc1nc(NC23CC(C#N)(C2)C3)nc2nn(C)c(Cl)c12]

(E)-3-((3-chloro-4-(4-(2-cyanovinyl)-2,6-dimethylphenoxy)-1-methyl-11H-pyrazolo[3,4-d]pyrimidin-6-yl)amino)bicyclo[1.1.1]pentane-1-carbonitrile—Compound 95 was prepared using the procedure described for the synthesis of Compound 92 and Compound 93 with the modification that Compound 94 was used in place of Compound 91 and the reaction was stirred at room temperature overnight. Compound 95 was the first eluted product and was the minor product. MS (m/z) 446.27 [M+H]+. ¹H NMR (400 MHz, Methanol-d4) δ 7.59-7.40 (m, 3H), 6.24 (d, J=16.6 Hz, 1H), 4.05 (s, 3H), 2.22-2.17 (m, 12H).

### Example 96

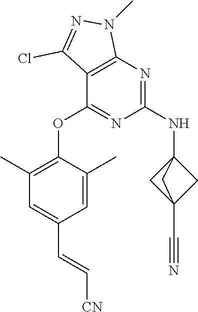 [SMILES: Cc1cc(/C=C/C#N)cc(C)c1Oc1nc(NC23CC(C#N)(C2)C3)nc2c1c(Cl)nn2C]

(E)-3-((3-chloro-4-(4-(2-cyanovinyl)-2,6-dimethylphenoxy)-1-methyl-11H-pyrazolo[3,4-d]pyrimidin-6-yl)amino)bicyclo[1.1.1]pentane-1-carbonitrile—Compound 96 was prepared using the procedure described for the synthesis of Compound 92 and Compound 93 with the modification that Compound 94 was used in place of Compound 91 and the reaction was stirred at rt overnight. Compound 96 was the second eluted product, and was the major product: MS (m/z) 446.12 [M+H]+. ¹H NMR (400 MHz, Methanol-d4) δ 7.62-7.34 (m, 3H), 6.23 (d, J=16.6 Hz, 1H), 3.84 (s, 3H), 2.48 (d, J=79.8 Hz, 2H), 2.25-2.11 (m, 10H).

### Example 97

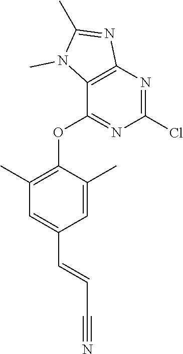 [SMILES: Cc1cc(/C=C/C#N)cc(C)c1Oc1nc(Cl)nc2nc(C)n(C)c12]

(E)-3-(4-((2-chloro-7,8-dimethyl-7H-purin-6-yl)oxy)-3,5-dimethylphenyl)acrylonitrile (Intermediate 97a) was prepared using the procedure described to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the modification that 2,6-dichloro-7,8-dimethyl-7H-purine was used in place of 2,6-dichloro-N-[(2,4-dimethoxyphenyl)methyl]-5-nitro-pyrimidin-4-amine.

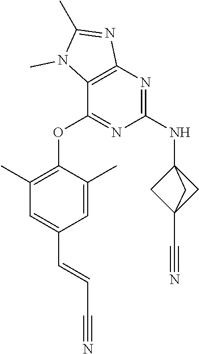 [SMILES: Cc1cc(/C=C/C#N)cc(C)c1Oc1nc(NC23CC(C#N)(C2)C3)nc2nc(C)n(C)c12]

(E)-3-((6-(4-(2-cyanovinyl)-2,6-dimethylphenoxy)-7,8-dimethyl-7H-purin-2-yl)amino)bicyclo[1.1.1]pentane-1-carbonitrile—Compound 97 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that Intermediate 97a was used in place of (E)-3-[4-[2-chloro-7-[(4-methoxyphenyl)methyl]-8-methyl-purin-6-yl]oxy-3,5-dimethyl-phenyl]prop-2-enenitrile. MS (m/z) 426.17 [M+H]+. ¹H NMR (400 MHz, Methanol-d4) δ 7.55 (d, J=16.7 Hz, 1H), 7.50 (s, 2H), 6.25 (d, J=16.6 Hz, 1H), 4.08 (s, 3H), 2.67 (s, 3H), 2.22 (s, 6H), 2.16 (s, 6H).

### Example 98

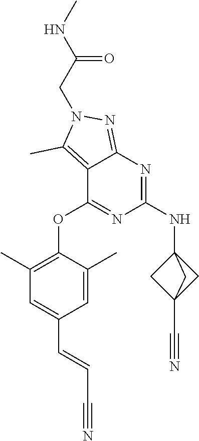 [SMILES: CNC(=O)Cn1nc2nc(NC34CC(C#N)(C3)C4)nc(Oc3c(C)cc(/C=C/C#N)cc3C)c2c1C]

(E)-2-(6-((3-cyanobicyclo[1.1.1]pentan-1-yl)amino)-4-(4-(2-cyanovinyl)-2,6-dimethylphenoxy)-3-methyl-2H-214-pyrazolo[3,4-d]pyrimidin-2-yl)-N-methylacetamide—Compound 98 was prepared using the procedure described for the synthesis of Compound 92 and Compound 93 with the modification that Compound 119 was used in place of Compound 91 and 2-bromo-N-methylacetamide was in place of iodomethane. Compound 98 was the first eluted product, and was the minor product isolated. MS (m/z) 483.30 [M+H]+. ¹H NMR (400 MHz, Methanol-d4) δ 7.54 (d, J=16.6 Hz, 1H), 7.48 (s, 2H), 6.24 (d, J=16.6 Hz, 1H), 4.99 (s, 2H), 2.81 (s, 3H), 2.78 (s, 3H), 2.23-2.11 (m, 12H).

### Example 99

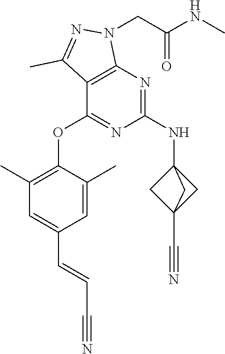

(E)-2-(6-((3-cyanobicyclo[1.1.1]pentan-1-yl)amino)-4-(4-(2-cyanovinyl)-2,6-dimethylphenoxy)-3-methyl-1H-pyrazolo[3,4-d]pyrimidin-1-yl)-N-methylacetamide—Compound 99 was prepared using the procedure described for the synthesis of Compound 92 and Compound 93 with the modification that Compound 119 was used in place of Compound 91 and 2-bromo-N-methylacetamide was in place of iodomethane. Compound 99 was the second eluting product and was the major isolated product. MS (m/z) 483.27 [M+H]+. ¹H NMR (400 MHz, Methanol-d4) δ 7.62-7.39 (m, 3H), 6.23 (d, J=16.7 Hz, 1H), 4.85 (s, 2H), 2.78 (s, 3H), 2.62 (s, 3H), 2.21-2.08 (m, 12H).

### Example 100

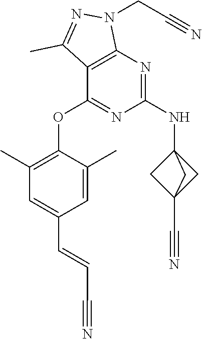 [SMILES: Cc1cc(/C=C/C#N)cc(C)c1Oc1nc(NC23CC(C#N)(C2)C3)nc2c1c(C)nn2CC#N]

(E)-3-((1-(cyanomethyl)-4-(4-(2-cyanovinyl)-2,6-dimethylphenoxy)-3-methyl-11H-pyrazolo[3,4-d]pyrimidin-6-yl)amino)bicyclo[1.1.1]pentane-1-carbonitrile—Compound 100 was prepared using the procedure described for the synthesis of Compound 92 and Compound 93 with the modification that Compound 119 was in place of (E)-3-((4-(4-(2-cyanovinyl)-2,6-dimethylphenoxy)-3-(difluoromethyl)-1H-pyrazolo[3,4-d]pyrimidin-6-yl)amino)bicyclo[1.1.1]pentane-1-carbonitrile (Compound 91) and bromoacetonitrile was in place of iodomethane. MS (m/z) 451.29 [M+H]+. ¹H NMR (400 MHz, Methanol-d4) δ 7.65-7.36 (m, 3H), 6.23 (d, J=16.7 Hz, 1H), 5.27 (s, 2H), 2.63 (d, J=4.3 Hz, 3H), 2.33-1.93 (m, 12H).

### Example 101

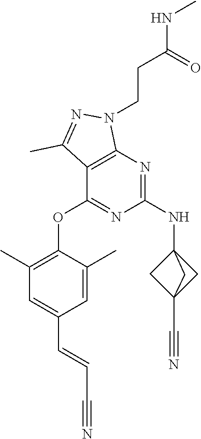 [SMILES: CNC(=O)CCn1nc(C)c2c(Oc3c(C)cc(/C=C/C#N)cc3C)nc(NC34CC(C#N)(C3)C4)nc21]

### (E)-3-(6-((3-cyanobicyclo[1.1.1]pentan-1-yl)amino)-4-(4-(2-cyanovinyl)-2,6-dimethylphenoxy)-3-methyl-1H-pyrazolo[3,4-d]pyrimidin-1-yl)-N-methylpropanamide (Compound 101)

To a solution of Compound 119 (25 mg, 0.061 mmol) in THE (2 mL) was added 3-Bromo-N-methylpropanamide (20 mg, 0.12 mmol) and NaH (7 mg, 0.18 mmol). The mixture was stirred at 60° C. for 8 hours. To the mixture was added water and the solution was extracted with EtOAc. The combined organic layers were dried over MgSO₄, filtered, concentrated in vacuo and subjected to silica gel flash column chromatography (0-100% hexane/EtOAc). The crude product was subjected to reverse HPLC (5-100% MeCN/H₂O). MS (m/z) 497.16 [M+H]+. ¹H NMR (400 MHz, Methanol-d4) δ 7.60-7.35 (m, 4H), 6.23 (dd, J=16.7, 3.6 Hz, 1H), 4.57-4.34 (m, 2H), 2.73 (t, J=7.0 Hz, 2H), 2.68 (s, 3H), 2.60 (s, 3H), 2.23-2.16 (m, 12H).

### Example 102

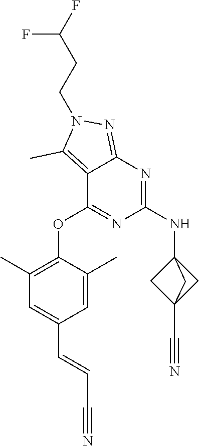 [SMILES: Cc1cc(/C=C/C#N)cc(C)c1Oc1nc(NC23CC(C#N)(C2)C3)nc2nn(CCC(F)F)c(C)c12]

(E)-3-((4-(4-(2-cyanovinyl)-2,6-dimethylphenoxy)-2-(3,3-difluoropropyl)-3-methyl-2H-pyrazolo[3,4-d]pyrimidin-6-yl)amino)bicyclo[1.1.1]pentane-1-carbonitrile (Compound 102) was prepared using the procedure described for the synthesis of Compound 102 with the modification that 3-bromo-1,1-difluoro-propane was in place of 3-Bromo-N-methylpropanamide. Compound 102 was the first eluting and minor product isolated. MS (m/z) 490.20 [M+H]+. ¹H NMR (400 MHz, Methanol-d4) δ 7.68-7.31 (m, 3H), 6.30-6.20 (m, 1H), 6.01 (dt, J=56.1, 4.3 Hz, 1H), 4.45 (t, J=6.9 Hz, 2H), 2.81 (d, J=4.3 Hz, 3H), 2.60-2.43 (m, 2H), 2.22-2.17 (m, 12H).

### Example 103

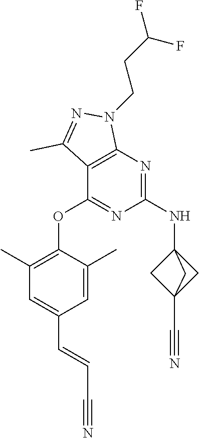 [SMILES: Cc1cc(/C=C/C#N)cc(C)c1Oc1nc(NC23CC(C#N)(C2)C3)nc2c1c(C)nn2CCC(F)F]

(E)-3-((4-(4-(2-cyanovinyl)-2,6-dimethylphenoxy)-1-(3,3-difluoropropyl)-3-methyl-1H-pyrazolo[3,4-d]pyrimidin-6-yl)amino)bicyclo[1.1.1]pentane-1-carbonitrile (Compound 103) was prepared using the procedure described for the synthesis of Compound 102 with the modification that 3-bromo-1,1-difluoro-propane was in place of 3-Bromo-N-methylpropanamide. Compound 103 was the second eluting and major product isolated. MS (m/z) 490.24 [M+H]+. ¹H NMR (400 MHz, Methanol-d4) δ 7.61-7.32 (m, 3H), 6.22 (d, J=16.7 Hz, 1H), 5.99 (tt, J=56.2, 4.4 Hz, 1H), 4.39 (t, J=6.8 Hz, 2H), 2.61 (s, 3H), 2.56-2.29 (m, 2H), 2.22-2.05 (m, 12H).

### Example 104

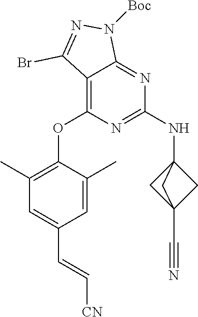 [SMILES: Cc1cc(/C=C/C#N)cc(C)c1Oc1nc(NC23CC(C#N)(C2)C3)nc2c1c(Br)nn2C(=O)OC(C)(C)C]

tert-butyl (E)-3-bromo-6-((3-cyanobicyclo[1.1.1]pentan-1-yl)amino)-4-(4-(2-cyanovinyl)-2,6-dimethylphenoxy)-1H-pyrazolo[3,4-d]pyrimidine-1-carboxylate (Intermediate 104a) was prepared using the three step procedure to synthesize Intermediate 11c, with the modification that 3-bromo-4-chloro-6-(methylthio)-1H-pyrazolo[3,4-d]pyrimidine was used in place of 4-chloro-6-(methylthio)-1H-pyrazolo[3,4-d]pyrimidine.

### Preparation of (E)-3-((4-(4-(2-cyanovinyl)-2,6-dimethylphenoxy)-3-cyclopropyl-1H-pyrazolo[3,4-d]pyrimidin-6-yl)amino)bicyclo[1.1.1]pentane-1- carbonitrile (Compound 104)

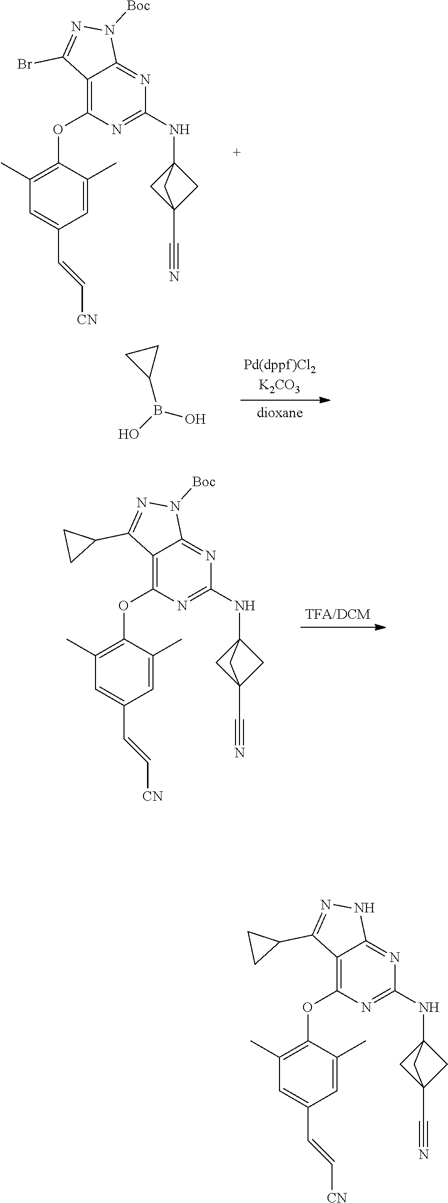 [SMILES: Cc1cc(/C=C/C#N)cc(C)c1Oc1nc(NC23CC(C#N)(C2)C3)nc2c1c(Br)nn2C(=O)OC(C)(C)C ; OB(O)C1CC1 ; Cc1cc(/C=C/C#N)cc(C)c1Oc1nc(NC23CC(C#N)(C2)C3)nc2c1c(C1CC1)nn2C(=O)OC(C)(C)C ; Cc1cc(/C=C/C#N)cc(C)c1Oc1nc(NC23CC(C#N)(C2)C3)nc2[nH]nc(C3CC3)c12]

Step 1: Preparation of tert-butyl (E)-6-((3-cyanobicyclo[1.1.1]pentan-1-yl)amino)-4-(4-(2-cyanovinyl)-2,6-dimethylphenoxy)-3-cyclopropyl-1H-pyrazolo[3,4-d]pyrimidine-1-carboxylate (Intermediate 104b): A mixture of (E)-3-bromo-6-((3-cyanobicyclo[1.1.1]pentan-1-yl)amino)-4-(4-(2-cyanovinyl)-2,6-dimethylphenoxy)-1H-pyrazolo[3,4-d]pyrimidine-1-carboxylate (Intermediate 105a) (25 mg, 0.043 mmol), cyclopropylboronic acid (18.6 mg, 0.217 mmol), [1,1′-Bis(diphenylphosphino)ferrocene] dichloropalladium(II) (10 mg, 0.013 mmol) and potassium carbonate (30 mg, 0.22 mmol) in dioxane (1 mL) was stirred at 85° C. for 2 hours. To the mixture was added more [1,1′-Bis(diphenylphosphino)ferrocene] dichloropalladium(II) (3.3 mg, 0.0042 mmol) and the mixture was stirred at 85° C. for another half hour. The mixture was cooled down to room temperature and to the mixture was added water. The solution was extracted with EtOAc and the organic phase was separated, dried over MgSO₄, filtered, and concentrated in vacuo. The resulting residue was subjected to silica gel flash column chromatography (0-100% hexane/EtOAc). MS (m/z) 537.84 [M+H]+.

Step 2: Preparation of (E)-3-((4-(4-(2-cyanovinyl)-2,6-dimethylphenoxy)-3-cyclopropyl-1H-pyrazolo[3,4-d]pyrimidin-6-yl)amino)bicyclo[1.1.1]pentane-1-carbonitrile A solution of (Intermediate 104b) in 1:1 DCM/TFA (1 mL) was stirred at room temperature for 1 hour. The mixture was concentrated in vacuo and the resulting residue was subjected to reverse phase HPLC (5-100% neutral MeCN/H₂O). MS (m/z) 438.26 [M+H]+. ¹H NMR (400 MHz, Methanol-d4) δ 7.54 (d, J=16.6 Hz, 1H), 7.48 (s, 2H), 6.24 (d, J=16.7 Hz, 1H), 2.43-2.35 (m, 1H), 2.29-2.09 (m, 12H), 1.09 (ddt, J=14.7, 6.5, 2.9 Hz, 4H).

### Example 105

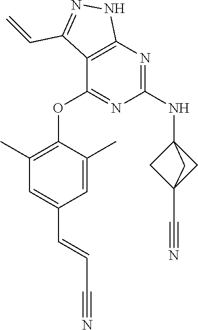 [SMILES: C=Cc1n[nH]c2nc(NC34CC(C#N)(C3)C4)nc(Oc3c(C)cc(/C=C/C#N)cc3C)c12]

(E)-3-((4-(4-(2-cyanovinyl)-2,6-dimethylphenoxy)-3-vinyl-1H-pyrazolo[3,4-d]pyrimidin-6-yl)amino)bicyclo[1.1.1]pentane-1-carbonitrile—Compound 105 was prepared using the procedure described to synthesize Compound 104, with the modification that 4,4,5,5-tetramethyl-2-vinyl-1,3,2-dioxaborolane was in place of cyclopropylboronic acid and dioxane/H2O (9/1) was in place of dioxane. MS (m/z) 424.22 [M+H]+. ¹H NMR (400 MHz, Methanol-d4) δ 7.60-7.43 (m, 3H), 6.99 (dd, J=17.9, 11.5 Hz, 1H), 6.46 (d, J=17.9 Hz, 1H), 6.24 (d, J=16.7 Hz, 1H), 5.58 (d, J=11.7 Hz, 1H), 2.26-2.16 (m, 12H).

### Example 106

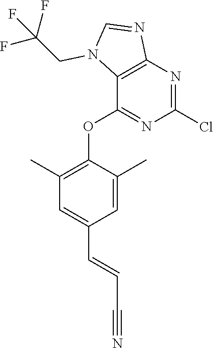 [SMILES: Cc1cc(/C=C/C#N)cc(C)c1Oc1nc(Cl)nc2ncn(CC(F)(F)F)c12]

(E)-3-(4-((2-chloro-7-(2,2,2-trifluoroethyl)-7H-purin-6-yl)oxy)-3,5-dimethylphenyl)acrylonitrile (Intermediate 106a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a), with the modification that 2,6-dichloro-7-(2,2,2-trifluoroethyl)-7H-purine was used in place of 2,6-dichloro-N-[(2,4-dimethoxyphenyl)methyl]-5-nitro-pyrimidin-4-amine.

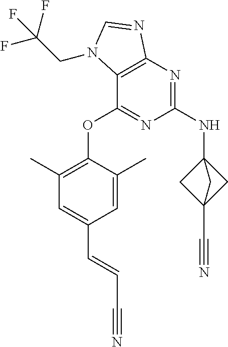 [SMILES: Cc1cc(/C=C/C#N)cc(C)c1Oc1nc(NC23CC(C#N)(C2)C3)nc2ncn(CC(F)(F)F)c12]

(E)-3-((6-(4-(2-cyanovinyl)-2,6-dimethylphenoxy)-7-(2,2,2-trifluoroethyl)-7H-purin-2-yl)amino)bicyclo[1.1.1]pentane-1-carbonitrile Compound 106 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that intermediate 106a was used in place of (E)-3-[4-[2-chloro-7-[(4-methoxyphenyl)methyl]-8-methyl-purin-6-yl]oxy-3,5-dimethyl-phenyl]prop-2-enenitrile. MS (m/z) 480.17 [M+H]+. ¹H NMR (400 MHz, Methanol-d4) δ 8.39 (s, 1H), 7.59-7.45 (m, 3H), 6.26 (d, J=16.7 Hz, 1H), 5.29 (q, J=8.6 Hz, 2H), 2.17 (d, J=11.9 Hz, 12H).

### Example 107

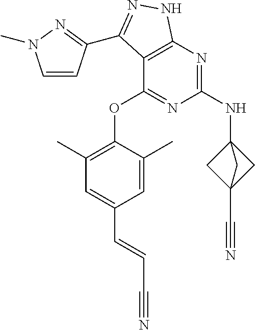 [SMILES: Cc1cc(/C=C/C#N)cc(C)c1Oc1nc(NC23CC(C#N)(C2)C3)nc2[nH]nc(-c3ccn(C)n3)c12]

(E)-3-((4-(4-(2-cyanovinyl)-2,6-dimethylphenoxy)-3-(1-methyl-1H-pyrazol-3-yl)-1H-pyrazolo[3,4-d]pyrimidin-6-yl)amino)bicyclo[1.1.1]pentane-1-carbonitrile—Compound 107 was prepared using the procedure described to synthesize Compound 104, with the modification that 1-methyl-3-(4,4,5,5-tetramethyl-1,3,2-dioxaborolan-2-yl)pyrazole was in place of cyclopropylboronic acid and dioxane/H₂O (9/1) was in place of dioxane. MS (m/z) 478.31 [M+H]+. ¹H NMR (400 MHz, Methanol-d4) δ 7.67 (d, J=2.4 Hz, 1H), 7.57-7.44 (m, 3H), 7.00 (d, J=2.3 Hz, 1H), 6.24 (d, J=16.6 Hz, 1H), 3.99 (s, 3H), 2.22-2.16 (m, 12H).

### Example 108

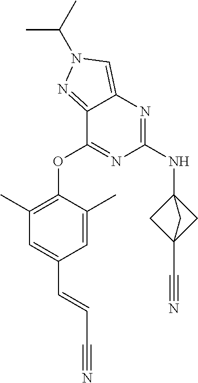 [SMILES: Cc1cc(/C=C/C#N)cc(C)c1Oc1nc(NC23CC(C#N)(C2)C3)nc2cn(C(C)C)nc12]

(E)-3-((4-(4-(2-cyanovinyl)-2,6-dimethylphenoxy)-3-(1-methyl-1H-pyrazol-3-yl)-112-pyrazolo[5,4-d]pyrimidin-6-yl)amino)bicyclo[1.1.1]pentane-1-carbonitrile—Compound 108 was prepared using the two step procedure described to synthesize Compound 4, with the modification that (E)-3-(4-hydroxy-3,5-dimethyl-phenyl)prop-2-enenitrile was used in place of (E)-3-(3-chloro-5-ethyl-4-hydroxyphenyl)acrylonitrile in the first step and the mixture was heated at 60° C. MS (m/z) 440.28 [M+H]+. ¹H NMR (400 MHz, Methanol-d4) δ 8.01 (s, 1H), 7.55 (d, J=16.7 Hz, 1H), 7.50 (s, 2H), 6.25 (d, J=16.6 Hz, 1H), 2.22-2.17 (m, 12H), 1.71 (d, J=6.7 Hz, 1H), 1.67 (d, J=6.7 Hz, 6H).

### Example 109

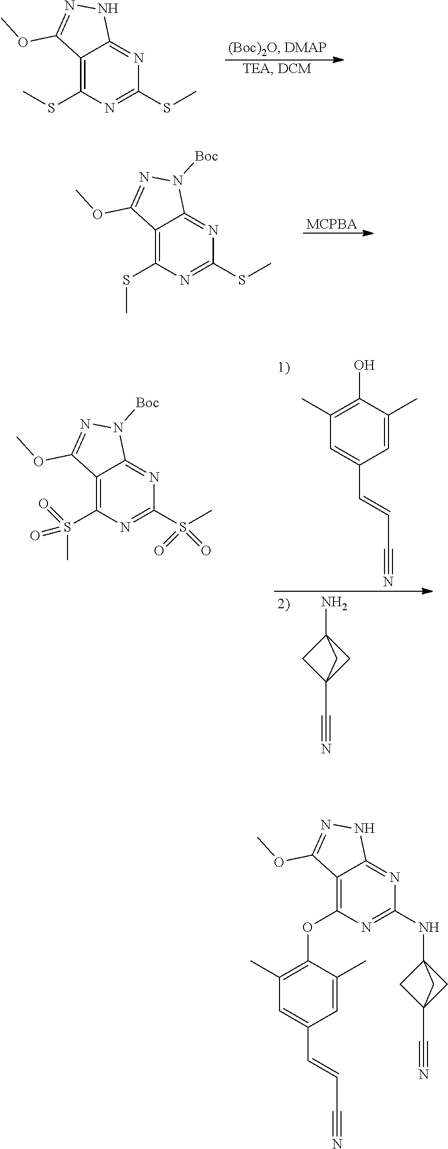

Step 1: preparation of tert-butyl 3-methoxy-4,6-bis(methylthio)-1H-pyrazolo[3,4-d]pyrimidine-1-carboxylate (Intermediate 109a): To a solution of 3-methoxy-4,6-bis(methylsulfanyl)-1H-pyrazolo[3,4-d]pyrimidine (1000 mg, 4.13 mmol) in DCM (10 mL), was added di-tert-butyl dicarbonate (1.6 g, 7.33 mmol), triethylamine (1.4 g, 14.3 mmol) and DMAP (50.4 mg, 0.413 mmol). The mixture was stirred at room temperature for 1 hour. The mixture was then concentrated in vacuo and the resulting residue was subjected to silica gel flash column chromatography (0-100% hexane/EtOAc).

Step 2: tert-butyl 3-methoxy-4,6-bis(methylsulfonyl)-1H-pyrazolo[3,4-d]pyrimidine-1-carboxylate (Intermediate 109b): To a solution of Intermediate 109a(355 mg, 1.04 mmol) in DCM (9 mL) was added MCPBA (805 mg, 4.67 mmol). The mixture was stirred at room temperature overnight. The mixture was then filtered and the filtrate was concentrated in vacuo and used directly in the next step without purification.

Step 3: preparation of (E)-3-((4-(4-(2-cyanovinyl)-2,6-dimethylphenoxy)-3-methoxy-1H-pyrazolo[3,4-d]pyrimidin-6-yl)amino)bicyclo[1.1.1]pentane-1-carbonitrile—Compound 109: To a solution of Intermediate 109b (277 mg. 0.682 mmol) in NMP was added potassium carbonate (474 mg, 3.41 mmol) and (E)-3-(4-hydroxy-3,5-dimethyl-phenyl)prop-2-enenitrile (18 mg, 0.102 mmol). The mixture was stirred at room temperature. After (E)-3-(4-hydroxy-3,5-dimethyl-phenyl)prop-2-enenitrile was fully consumed, to the mixture was added 3-aminobicyclo[1.1.1]pentane-1-carbonitrile hydrochloride (98 mg, 0.68 mmol). The mixture was stirred at room temperature overnight then concentrated in vacuo onto silica gel and subjected to silica gel flash column chromatography (0-100% hexane/EtOAc) to give the title compound. MS (m/z) 428.16 [M+H]+. ¹H NMR (400 MHz, Methanol-d₄) δ 7.53 (d, J=16.7 Hz, 1H), 7.46 (s, 2H), 6.23 (d, J=16.7 Hz, 1H), 4.06 (s, 3H), 2.23-2.06 (m, 12H).

### Example 110

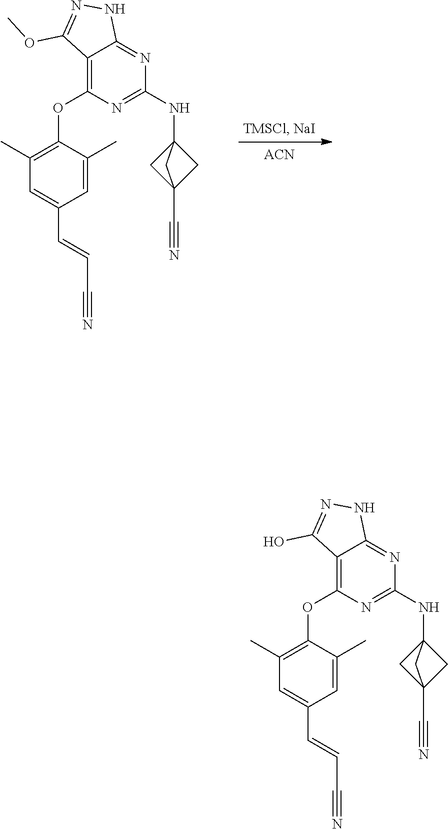

Preparation of (E)-3-((4-(4-(2-cyanovinyl)-2,6-dimethylphenoxy)-3-hydroxy-1H-pyrazolo[3,4-d]pyrimidin-6-yl)amino)bicyclo[1.1.1]pentane-1-carbonitrile—Compound 110: The reaction mixture of Compound 109 (27 mg, 0.063 mmol), sodium iodide (47 mg, 0.32 mmol) and chlorotrimethylsilane (34 mg, 0.322 mmol) in acetonitrile (2 mL) was stirred at 55° C. for 2 hours. The reaction mixture was filtered and the filtrate was subjected to reverse phase HPLC (5-100% neutral MeCN/water) and lyophilized to afford the title compound. MS (m/z) 414.15 [M+H]+. ¹H NMR (400 MHz, Methanol-d4) δ 7.65-7.36 (m, 3H), 6.22 (d, J=16.7 Hz, 1H), 2.25-2.06 (m, 12H).

### Example 111

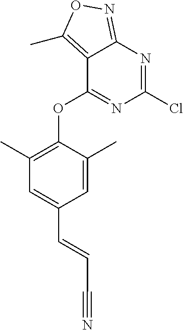 [SMILES: Cc1cc(/C=C/C#N)cc(C)c1Oc1nc(Cl)nc2noc(C)c12]

(E)-3-(4-((6-chloro-3-methylisoxazolo[3,4-d]pyrimidin-4-yl)oxy)-3,5-dimethylphenyl)acrylonitrile (Intermediate 111a) was prepared using the procedure described for the synthesis of (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the modification that 4,6-dichloro-3-methylisoxazolo[3,4-d]pyrimidine was used in place of 2,6-dichloro-N-[(2,4-dimethoxyphenyl)methyl]-5-nitro-pyrimidin-4-amine and the reaction was stirred at room temperature for 10 minutes.

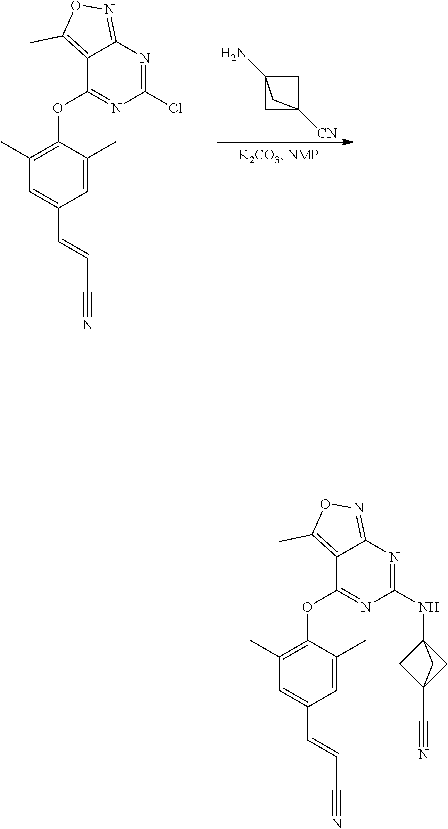

Preparation of (E)-3-((4-(4-(2-cyanovinyl)-2,6-dimethylphenoxy)-3-methylisoxazolo[3,4-d]pyrimidin-6-yl)amino)bicyclo[1.1.1]pentane-1-carbonitrile—Compound 111: To a flask charged with Intermediate 111a (180 mg, 0.53 mmol), potassium carbonate (219 mg, 1.58 mmol) and 3-aminobicyclo[1.1.1]pentane-1-carbonitrile (114 mg, 1.06 mmol) was added NMP (4 mL). The mixture was heated at 55° C. for 1 hour. The mixture was cooled to room temperature and directly subjected to silica gel flash column chromatography (0-100% hexane/EtOAc). The crude product was subjected to reverse phase HPLC, (5-100% neutral MeCN/water) and lyophilized to afford the title compound. MS (m/z) 413.14 [M+H]+. ¹H NMR (400 MHz, Acetonitrile-d3) δ 7.46 (m, 3H), 6.10 (d, J=16.7 Hz, 1H), 2.87 (s, 3H), 2.22-2.04 (m, 12H).

### Example 112

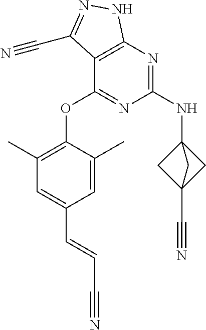 [SMILES: Cc1cc(/C=C/C#N)cc(C)c1Oc1nc(NC23CC(C#N)(C2)C3)nc2[nH]nc(C#N)c12]

(E)-6-((3-cyanobicyclo[1.1.1]pentan-1-yl)amino)-4-(4-(2-cyanovinyl)-2,6-dimethylphenoxy)-1H-pyrazolo[3,4-d]pyrimidine-3-carbonitrile—Compound 112 was prepared using the procedure described for the synthesis of Compound 10, with the modification that 4-chloro-6-methylsulfanyl-1H-pyrazolo[3,4-d]pyrimidine-3-carbonitrile was used in place of 4-chloro-6-(methylthio)-1H-pyrazolo[3,4-d]pyrimidine. MS (m/z) 423.08 [M+H]+. ¹H NMR (400 MHz, Methanol-d4) δ 7.54 (d, J=17.7 Hz, 3H), 6.25 (d, J=16.6 Hz, 1H), 2.57-2.02 (m, 12H).

### Example 113

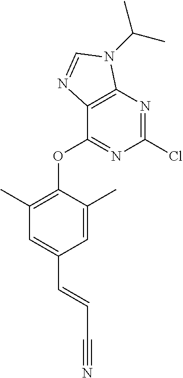 [SMILES: Cc1cc(/C=C/C#N)cc(C)c1Oc1nc(Cl)nc2c1ncn2C(C)C]

(E)-3-(4-((2-chloro-9-isopropyl-9H-purin-6-yl)oxy)-3,5-dimethylphenyl)acrylonitrile (Intermediate 113a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the modification that 2,6-dichloro-9-isopropyl-9H-purine was used in place of 2,6-dichloro-N-[(2,4-dimethoxyphenyl)methyl]-5-nitro-pyrimidin-4-amine.

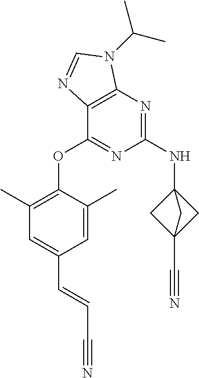 [SMILES: Cc1cc(/C=C/C#N)cc(C)c1Oc1nc(NC23CC(C#N)(C2)C3)nc2c1ncn2C(C)C]

(E)-3-((6-(4-(2-cyanovinyl)-2,6-dimethylphenoxy)-9-isopropyl-9H-purin-2-yl)amino)bicyclo[1.1.1]pentane-1-carbonitrile—Compound 113 was prepared using the procedure described for the synthesis of int-x, with the modification that Intermediate 113a was used in place of Int-xx. MS (m/z) 440.16 [M+H]+. ¹H NMR (400 MHz, Methanol-d4) δ 8.52 (s, 1H), 7.58-7.44 (m, 3H), 6.24 (d, J=16.6 Hz, 1H), 4.82 (q, J=6.7 Hz, 1H), 2.30-2.14 (m, 12H), 1.66 (d, J=6.8 Hz, 6H).

### Example 114

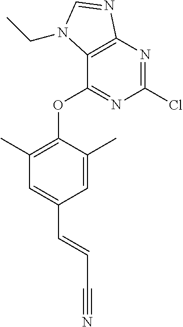 [SMILES: CCn1cnc2nc(Cl)nc(Oc3c(C)cc(/C=C/C#N)cc3C)c21]

(E)-3-(4-((2-chloro-7-ethyl-7H-purin-6-yl)oxy)-3,5-dimethylphenyl)acrylonitrile (Intermediate 114a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a), with the modification that 2,6-dichloro-7-ethyl-7H-purine was used in place of 2,6-dichloro-N-[(2,4-dimethoxyphenyl)methyl]-5-nitro-pyrimidin-4-amine.

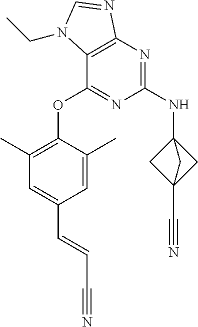 [SMILES: CCn1cnc2nc(NC34CC(C#N)(C3)C4)nc(Oc3c(C)cc(/C=C/C#N)cc3C)c21]

(E)-3-((6-(4-(2-cyanovinyl)-2,6-dimethylphenoxy)-7-ethyl-7H-purin-2-yl)amino)bicyclo[1.1.1]pentane-1-carbonitrile—Compound 114 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that Intermediate 114a was used in place of (E)-3-[4-[2-chloro-7-[(4-methoxyphenyl)methyl]-8-methyl-purin-6-yl]oxy-3,5-dimethyl-phenyl]prop-2-enenitrile. MS (m/z) 426.15 [M+H]+. ¹H NMR (400 MHz, Methanol-d4) δ 8.61 (s, 1H), 7.61-7.45 (m, 3H), 6.26 (d, J=16.6 Hz, 1H), 4.55 (q, J=7.2 Hz, 2H), 2.24-2.18 (m, 12H), 1.67 (t, J=7.2 Hz, 3H).

### Example 115

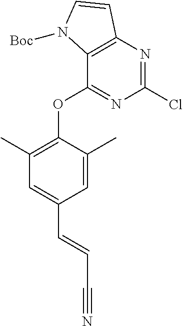 [SMILES: Cc1cc(/C=C/C#N)cc(C)c1Oc1nc(Cl)nc2ccn(C(=O)OC(C)(C)C)c12]

tert-butyl (E)-2-chloro-4-(4-(2-cyanovinyl)-2,6-dimethylphenoxy)-5H-pyrrolo[3,2-d]pyrimidine-5-carboxylate (Intermediate 115a) was synthesized using the procedure described for the synthesis of Intermediate 1f, with the modification that 2,4-dichloro-5H-pyrrolo[3,2-d]pyrimidine was used in place of €-3-(3-chloro-5-ethyl-4-hydroxyphenyl)acrylonitrile and the crude product from the first reaction was subjected to silica gel flash column chromatography(0-100% hexane/EtOAc).

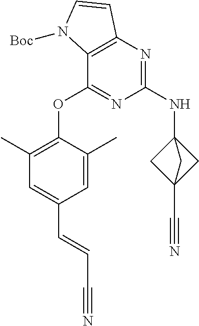 [SMILES: Cc1cc(/C=C/C#N)cc(C)c1Oc1nc(NC23CC(C#N)(C2)C3)nc2ccn(C(=O)OC(C)(C)C)c12]

(E)-3-((4-(4-(2-cyanovinyl)-2,6-dimethylphenoxy)-5H-pyrrolo[3,2-d]pyrimidin-2-yl)amino)bicyclo[1.1.1]pentane-1-carbonitrile (Intermediate 115b) was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that intermediate 115b was used in place of (E)-3-[4-[2-chloro-7-[(4-methoxyphenyl)methyl]-8-methyl-purin-6-yl]oxy-3,5-dimethyl-phenyl]prop-2-enenitrile, except that the reaction was heated at 120° C.

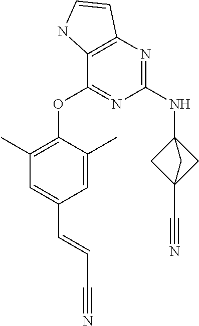 [SMILES: Cc1cc(/C=C/C#N)cc(C)c1Oc1nc(NC23CC(C#N)(C2)C3)nc2cc[nH]c12]

Preparation of (E)-3-((4-(4-(2-cyanovinyl)-2,6-dimethylphenoxy)-5H-pyrrolo[3,2-d]pyrimidin-2-yl)amino)bicyclo[1.1.1]pentane-1-carbonitrile—Compound 115: Intermediate 116b in 1:1 DCM/TFA (1 mL) was stirred at room temperature for 1 hour. The mixture was concentrated in vacuo and the residue was subjected to reverse phase HPLC (5-100% MeCN/H₂O, containing 0.1% TFA) and lyophilized to afford the title compound. MS (m/z) 397.18 [M+H]+. ¹H NMR (400 MHz, Methanol-d4) δ 7.82 (d, J=2.9 Hz, 1H), 7.60-7.54 (m, 3H), 6.51 (d, J=2.9 Hz, 1H), 6.29 (d, J=16.7 Hz, 1H), 2.26-2.10 (m, 12H).

### Example 116

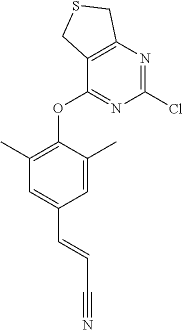 [SMILES: Cc1cc(/C=C/C#N)cc(C)c1Oc1nc(Cl)nc2c1CSC2]

(E)-3-(4-((2-chloro-5,7-dihydrothieno[3,4-d]pyrimidin-4-yl)oxy)-3,5-dimethylphenyl)acrylonitrile (Intermediate 116a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a), with the modification that 2,4-dichloro-5,7-dihydrothieno[3,4-d]pyrimidine was used in place of 2,6-dichloro-N-[(2,4-dimethoxyphenyl)methyl]-5-nitro-pyrimidin-4-amine.

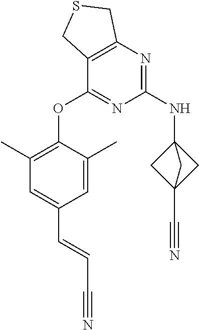 [SMILES: Cc1cc(/C=C/C#N)cc(C)c1Oc1nc(NC23CC(C#N)(C2)C3)nc2c1CSC2]

(E)-3-((4-(4-(2-cyanovinyl)-2,6-dimethylphenoxy)-5,7-dihydrothieno[3,4-d]pyrimidin-2-yl)amino)bicyclo[1.1.1]pentane-1-carbonitrile—Compound 116 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that Intermediate 81a was used in place of (E)-3-[4-[2-chloro-7-[(4-methoxyphenyl)methyl]-8-methyl-purin-6-yl]oxy-3,5-dimethyl-phenyl]prop-2-enenitrile, except that the reaction was heated at 120° C. MS (m/z) 416.12 [M+H]+. ¹H NMR (400 MHz, Methanol-d4) δ 7.53 (d, J=16.7 Hz, 1H), 7.47 (s, 2H), 6.23 (d, J=16.6 Hz, 1H), 4.22 (t, J=2.3 Hz, 2H), 4.12 (t, J=2.3 Hz, 2H), 2.16-2.11 (m, 12H).

### Example 117

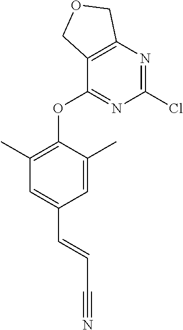 [SMILES: Cc1cc(/C=C/C#N)cc(C)c1Oc1nc(Cl)nc2c1COC2]

(E)-3-(4-((2-chloro-5,7-dihydrofuro[3,4-d]pyrimidin-4-yl)oxy)-3,5-dimethylphenyl)acrylonitrile (Intermediate 117a) was prepared using the procedure prescribed to synthesize (E)-3-(4-((2-chloro-6-((2,4-dimethoxybenzyl)amino)-5-nitropyrimidin-4-yl)oxy)-3,5-dimethylphenyl)acrylonitrile (Intermediate 176a), with the modification that 2,4-dichloro-5,7-dihydrofuro[3,4-d]pyrimidine was used in place of 2,6-dichloro-N-[(2,4-dimethoxyphenyl)methyl]-5-nitro-pyrimidin-4-amine.

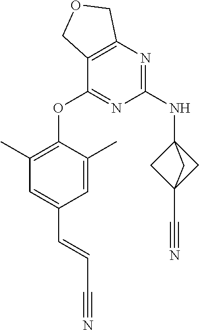 [SMILES: Cc1cc(/C=C/C#N)cc(C)c1Oc1nc(NC23CC(C#N)(C2)C3)nc2c1COC2]

Compound 117 was prepared using the procedure described for the synthesis of (E)-3-((6-(4-(2-cyanovinyl)-2,6-dimethylphenoxy)-7-(4-methoxybenzyl)-8-methyl-7H-purin-2-yl)amino)bicyclo[1.1.1]pentane-1-carbonitrile, with the modification that Intermediate 117a was used in place of (E)-3-[4-[2-chloro-7-[(4-methoxyphenyl)methyl]-8-methyl-purin-6-yl]oxy-3,5-dimethyl-phenyl]prop-2-enenitrile, except that the reaction was heated at 120° C. MS (m/z) 400.19 [M+H]+. Multiplet Report ¹H NMR (400 MHz, Methanol-d4) δ 7.53 (d, J=16.7 Hz, 1H), 7.47 (s, 2H), 6.23 (d, J=16.6 Hz, 1H), 5.13 (t, J=2.3 Hz, 2H), 4.84 (t, J=2.2 Hz, 2H), 2.12-2.11 (m, 12H).

### Example 118

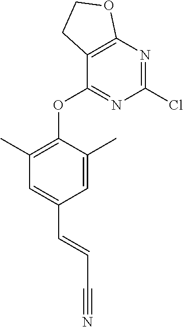 [SMILES: Cc1cc(/C=C/C#N)cc(C)c1Oc1nc(Cl)nc2c1CCO2]

(E)-3-(4-((2-chloro-5,6-dihydrofuro[2,3-d]pyrimidin-4-yl)oxy)-3,5-dimethylphenyl)acrylonitrile (Intermediate 118a) was prepared using the procedure prescribed to synthesize(E)-3-(4-((2-chloro-6-((2,4-dimethoxybenzyl)amino)-5-nitropyrimidin-4-yl)oxy)-3,5-dimethylphenyl)acrylonitrile (Intermediate 176a), with the modification that 2,4-dichloro-5,6-dihydrofuro[2,3-d]pyrimidine was used in place of 2,6-dichloro-N-[(2,4-dimethoxyphenyl)methyl]-5-nitro-pyrimidin-4-amine.

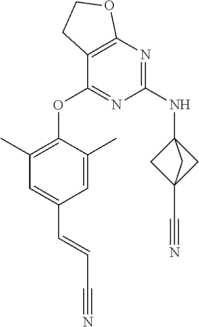 [SMILES: Cc1cc(/C=C/C#N)cc(C)c1Oc1nc(NC23CC(C#N)(C2)C3)nc2c1CCO2]

(E)-3-((4-(4-(2-cyanovinyl)-2,6-dimethylphenoxy)-5,6-dihydrofuro[2,3-d]pyrimidin-2-yl)amino)bicyclo[1.1.1]pentane-1-carbonitrile—Compound 118: A solution of Intermediate 118a (30 mg, 0.0915 mmol), 3-aminobicyclo[1.1.1]pentane-1-carbonitrile (14.8 mg, 0.137 mmol) in NMP (0.5 mL) was heated 140° C. overnight. The mixture was cooled to room temperature and subjected to reverse phase HPLC (5-100% MeCN/H₂O, containing 0.1% TFA). MS (m/z) 400.16 [M+H]+. H NMR (400 MHz, Acetonitrile-d₃) δ 7.49 (d, J=16.7 Hz, 1H), 7.38 (s, 2H), 6.11 (s, 1H), 6.07 (d, J=16.7 Hz, 1H), 4.64 (t, J=8.6 Hz, 2H), 3.15 (t, J=8.5 Hz, 2H), 2.20-2.14 (m, 12H).

### Example 119

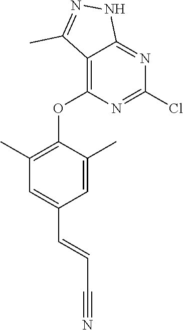 [SMILES: Cc1cc(/C=C/C#N)cc(C)c1Oc1nc(Cl)nc2[nH]nc(C)c12]

Preparation of (E)-3-[4-[(6-chloro-3-methyl-1H-pyrazolo[3,4-d]pyrimidin-4-yl)oxy]-3,5-dimethyl-phenyl]prop-2-enenitrile (Intermediate 119a): A mixture of 4,6-dichloro-3-methyl-1H-pyrazolo[3,4-d]pyrimidine (0.25 g, 1.23 mmol), (E)-3-(4-hydroxy-3,5-dimethyl-phenyl)prop-2-enenitrile (427 mg, 2.46 mmol), and potassium carbonate (0.17 g, 1.23 mmol) in DMF (5 mL) was stirred at room temperature for four hours. The mixture was then diluted with EtOAc and washed with water. The organic phase was dried over MgSO₄, filtered, and concentrated in vacuo. The resulting residue was purified by silica gel chromatography (0-100% EtOAc/hexanes) to afford the title compound. MS (m/z) 340.09 [M+H]+. ¹H NMR (400 MHz, Chloroform-d) δ 7.37 (d, J=16.7 Hz, 1H), 7.24 (s, 2H), 5.86 (d, J=16.6 Hz, 1H), 2.73 (s, 3H), 2.17 (s, 6H).

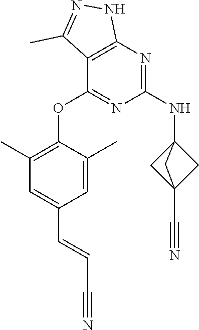 [SMILES: Cc1cc(/C=C/C#N)cc(C)c1Oc1nc(NC23CC(C#N)(C2)C3)nc2[nH]nc(C)c12]

Preparation of 3-[[4-[4-[(E)-2-cyanovinyl]-2,6-dimethyl-phenoxy]-3-methyl-1H-pyrazolo[3,4-d]pyrimidin-6-yl]amino]bicyclo[11.1.1]pentane-1-carbonitrile—Compound 119: 3-aminobicyclo[1.1.1]pentane-1-carbonitrile (0.261 g, 2.42 mmol), Intermediate 119a (205 mg, 0.603 mmol) and diisopropylethylamine (0.315 mL, 1.81 mmol) were suspended in NMP (6 mL). The mixture was stirred under nitrogen at 145° C. for 1.5 hr. Then more 3-aminobicyclo[1.1.1]pentane-1-carbonitrile (0.261 g, 2.42 mmol) was added. The mixture was stirred under nitrogen at 145° C. for 1.5 hr. The mixture was diluted with DMF, filtered, and purified via preparative HPLC (0-100% MeCN in water) then lyophilized to afford the title compound. MS (m/z) 412.17 [M+H]+. ¹H NMR (400 MHz, Acetonitrile-d3) δ 10.65 (s, 1H), 7.48 (d, J=16.7 Hz, 1H), 7.39 (s, 2H), 6.22 (s, 1H), 6.06 (d, J=16.7 Hz, 1H), 2.56 (s, 3H), 2.18-2.11 (m, 12H).

### Example 120

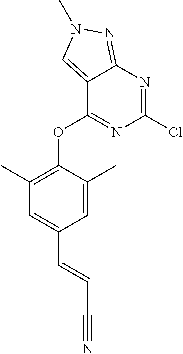 [SMILES: Cc1cc(/C=C/C#N)cc(C)c1Oc1nc(Cl)nc2nn(C)cc12]

### (E)-3-[4-(6-chloro-2-methyl-pyrazolo[3,4-d]pyrimidin-4-yl)oxy-3,5-dimethyl-phenyl]prop-2-enenitrile (Intermediate 120a)

4,6-Dichloro-2-methyl-pyrazolo[3,4-d]pyrimidine (1.00 g, 4.93 mmol), (E)-3-(4-hydroxy-3,5-dimethyl-phenyl)prop-2-enenitrile (853 mg, 4.93 mmol), and potassium carbonate (681 mg, 4.93 mmol) were mixed in DMF (20 mL). The mixture was stirred at room temperature for 21 hours. Water (40 mL) was added to the mixture. The mixture was stirred and the resulting solid collected by filtration to afford the title compound. MS (m/z) 340.11 [M+H]+. ¹H NMR (400 MHz, DMSO-d6) δ 8.79 (s, 1H), 7.63 (d, J=16.7 Hz, 1H), 7.53 (s, 2H), 6.46 (d, J=16.7 Hz, 1H), 4.20 (s, 3H), 2.09 (s, 6H).

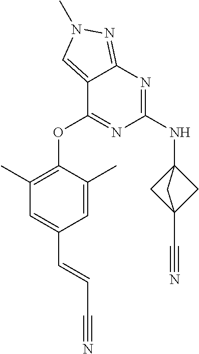 [SMILES: Cc1cc(/C=C/C#N)cc(C)c1Oc1nc(NC23CC(C#N)(C2)C3)nc2nn(C)cc12]

3-[[4-[4-[(E)-2-cyanovinyl]-2,6-dimethyl-phenoxy]-2-methyl-pyrazolo[3,4-d]pyrimidin-6-yl]amino]bicyclo[1.1.1]pentane-1-carbonitrile—Compound 120 (75 mg, 0.221 mmol), 3-aminobicyclo[1.1.1]pentane-1-carbonitrile (72.1 mg, 0.662 mmol), rac BINAP Pd G3 (110 mg, 0.110 mmol), and cesium carbonate (216 mg, 0.662 mmol) were combined in a microwave vial and 1,4-dioxane (3.3 mL) was added. The mixture was degassed with N₂ and then the vial sealed and heated to 50° C. for 60 h. The mixture was then cooled to room temperature, diluted with ethyl acetate, filtered and concentrated in vacuo. The resulting residue was purified by preparative HPLC (0-100% MeCN/water, 0.1% TFA). HPLC fractions containing product were collected, partitioned between water and EtOAc, and treated with 1.1 equivalents of solid NaHCO₃ relative to the amount of TFA present in the fraction volumes (assuming 0.1% TFA). The organic phase was washed with more water, then dried over MgSO₄, filtered, and concentrated in vacuo to afford the title compound. MS (m/z) 412.24 [M+H]+. ¹H NMR (400 MHz, Acetonitrile-d3) δ 8.00 (s, 1H), 7.48 (d, J=16.7 Hz, 1H), 7.38 (s, 2H), 6.14-5.94 (m, 2H), 4.02 (s, 3H), 2.13 (s, 6H), 2.11 (s, 6H).

### Example 121

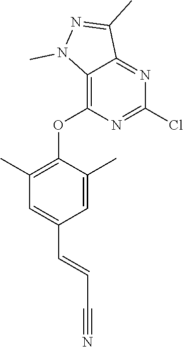 [SMILES: Cc1cc(/C=C/C#N)cc(C)c1Oc1nc(Cl)nc2c(C)nn(C)c12]

(E)-3-[4-(5-chloro-1,3-dimethyl-pyrazolo[4,3-d]pyrimidin-7-yl)oxy-3,5-dimethyl-phenyl]prop-2-enenitrile (Intermediate 121a) was prepared in a manner similar to Intermediate 121a, substituting 5,7-dichloro-1,3-dimethyl-pyrazolo[4,3-d]pyrimidine in place of 4,6-dichloro-3-methyl-1H-pyrazolo[3,4-d]pyrimidine. MS (m/z) 354.18 [M+H]+. ¹H NMR (400 MHz, Acetonitrile-d3) δ 7.48 (d, J=16.7 Hz, 1H), 7.40 (s, 2H), 6.09 (d, J=16.7 Hz, 1H), 4.28 (s, 3H), 2.51 (s, 3H), 2.16 (s, 6H).

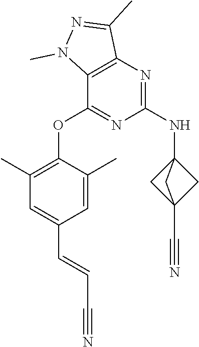 [SMILES: Cc1cc(/C=C/C#N)cc(C)c1Oc1nc(NC23CC(C#N)(C2)C3)nc2c(C)nn(C)c12]

(E)-3-[4-(5-chloro-1,3-dimethyl-pyrazolo[4,3-d]pyrimidin-7-yl)oxy-3,5-dimethyl-phenyl]prop-2-enenitrile—Compound 121 was prepared in a manner similar to Compound 120, substituting Intermediate 121a in place of Intermediate 120a. MS (m/z) 426.28 [M+H]+. ¹H NMR (400 MHz, Acetonitrile-d3) δ 7.49 (d, J=16.7 Hz, 1H), 7.40 (s, 2H), 6.07 (d, J=16.7 Hz, 1H), 5.95 (s, 1H), 4.18 (s, 3H), 2.38 (s, 3H), 2.14 (s, 12H).

### Example 122

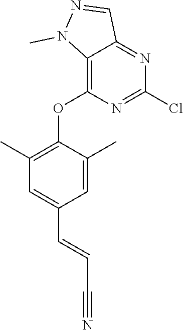 [SMILES: Cc1cc(/C=C/C#N)cc(C)c1Oc1nc(Cl)nc2cnn(C)c12]

(E)-3-[4-(5-chloro-1-methyl-pyrazolo[4,3-d]pyrimidin-7-yl)oxy-3,5-dimethyl-phenyl]prop-2-enenitrile (Intermediate 122a) was prepared in a manner similar to Intermediate 120a, substituting 5,7-dichloro-1-methyl-pyrazolo[4,3-d]pyrimidine in place of 4,6-dichloro-3-methyl-1H-pyrazolo[3,4-d]pyrimidine. MS (m/z) 340.10 [M+H]+. ¹H NMR (400 MHz, Chloroform-d) δ 8.11 (s, 1H), 7.38 (d, J=16.6 Hz, 1H), 7.26 (s, 2H), 5.88 (d, J=16.6 Hz, 1H), 4.41 (s, 3H), 2.19 (s, 6H).

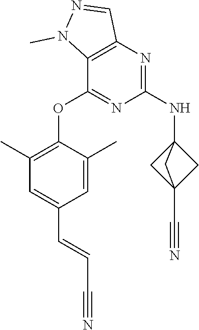 [SMILES: Cc1cc(/C=C/C#N)cc(C)c1Oc1nc(NC23CC(C#N)(C2)C3)nc2cnn(C)c12]

(E)-3-[4-(5-chloro-1-methyl-pyrazolo[4,3-d]pyrimidin-7-yl)oxy-3,5-dimethyl-phenyl]prop-2-enenitrile—Compound 122 was prepared in a manner similar to Compound 120, substituting Intermediate 122a in place of Intermediate 120a and running for only 2 hr. MS (m/z) 412.18 [M+H]+. ¹H NMR (400 MHz, Acetonitrile-d3) δ 7.70 (s, 1H), 7.49 (d, J=16.7 Hz, 1H), 7.41 (s, 2H), 6.08 (d, J=16.7 Hz, 1H), 6.04 (s, 1H), 4.25 (s, 3H), 2.17-2.14 (m, 12H).

### Example 123

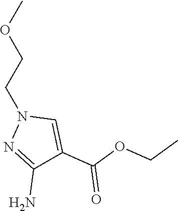

### Ethyl 3-amino-1-(2-methoxyethyl)-1H-pyrazole-4-carboxylate (Intermediate 123a)

(2-methoxyethyl)hydrazine dihydrochloride (15.0 g, 92.0 mmol) and anisaldehyde (12.5 g, 92.0 mmol) in THE (300 mL) was stirred at 70° C. for 3 hours. The mixture was then cooled down to room temperature and concentrated under reduced pressure. The residue was mixed with toluene (300 mL), then ethyl (2E)-2-cyano-3-ethoxyprop-2-enoate (15.5 g, 92.0 mmol). The mixture was stirred at 110° C. for 1 hour. The mixture was then cooled down to room temperature and concentrated under reduced pressure. Silica gel flash column chromatography (petroleum ether/EtOAc) afforded a product which was then mixed with EtOH (100 mL), followed by concentrated HCl (6.04 mL). The mixture was stirred for 2 hours at 80° C. The mixture was then cooled to room temperature and concentrated under reduced pressure. The residue was diluted with saturated aqueous sodium bicarbonate and extracted with EtOAc. The combined organic layer was dried over anhydrous Na₂SO₄, concentrated under vacuum. The crude product was then purified through silica gel column chromatography (DCM/EtOAc) to afford the title compound. LCMS (m/z): 214.00 [M+H]+.

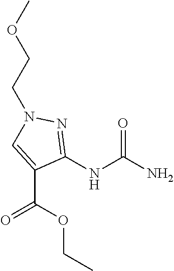 [SMILES: CCOC(=O)c1cn(CCOC)nc1NC(N)=O]

### ethyl 1-(2-methoxyethyl)-3-ureido-1H-pyrazole-4-carboxylate (Intermediate 123b)

To ethyl 3-amino-1-(2-methoxyethyl)pyrazole-4-carboxylate (5.70 g, 26.7 mmol) in AcOH (60 mL) was added potassium cyanate (8.67 g, 106 mmol). The resulting mixture was stirred for 2 h at room temperature. The mixture was adjusted to pH 8 with saturated aqueous NaHCO3. The resulting mixture was extracted with EtOAc. The combined organic layers were dried over anhydrous Na₂SO₄. After filtration, the filtrate was concentrated under reduced pressure. The residue was purified by silica gel column chromatography (CH2Cl2/EtOAc) to afford the title compound. LCMS (m/z): 257.00 [M+H]+

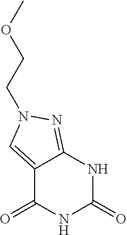

### 2-(2-methoxyethyl)-2,7-dihydro-4H-pyrazolo[3,4-d]pyrimidine-4,6(5H)-dione (Intermediate 123c)

A mixture of ethyl 1-(2-methoxyethyl)-3-ureido-1H-pyrazole-4-carboxylate (3.80 g, 14.8 mmol) and EtONa (2.02 g, 29.6 mmol) in EtOH (30.0 mL) was stirred for 2 h at 80° C. The mixture was cooled to room temperature and concentrated under vacuum. The mixture diluted with water, acidified to pH 5 with concentrated HCl and extracted with EtOAc. The combined organic layers were dried over anhydrous sodium sulfate and concentrated under reduced pressure. The residue was purified by silica gel column chromatography (DCM/MeOH) to afford the title compound. LCMS (m/z): 211.00 [M+H]+.

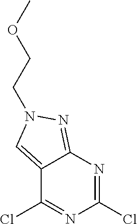

### 4,6-dichloro-2-(2-methoxyethyl)pyrazolo[3,4-d]pyrimidine (Intermediate 123d)

A mixture of 2-(2-methoxyethyl)-5H,7H-pyrazolo[3,4-d]pyrimidine-4,6-dione (3.00 g, 12.8 mmol) in POCl₃ (50.0 mL) was stirred at 100° C. for 16 hours. The mixture was cooled down to room temperature and concentrated under reduced pressure. The residue was diluted with EtOAc and diluted with saturated aqueous NaHCO₃. The mixture was extracted with EtOAc, dried over anhydrous sodium sulfate and concentrated under reduced pressure. The crude product was purified by silica gel column chromatography (petroleum ether/ethyl acetate) to afford the title compound. ¹H NMR (400 MHz, Chloroform-d) δ 8.27 (s, 1H), 4.70-4.50 (m, 2H), 3.90 (dd, J=5.4, 4.3 Hz, 2H), 3.35 (s, 3H). LCMS (m/z): 247.05 [M+H]+.

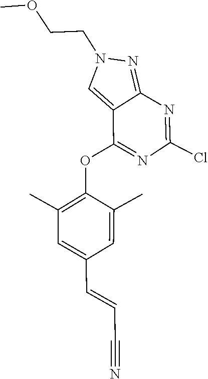 [SMILES: COCCn1cc2c(Oc3c(C)cc(/C=C/C#N)cc3C)nc(Cl)nc2n1]

### (E)-3-[4-[6-chloro-2-(2-methoxyethyl)pyrazolo[3,4-d]pyrimidin-4-yl]oxy-3,5-dimethyl-phenyl]prop-2-enenitrile (Intermediate 123e)

The title compound was prepared in a manner similar to Intermediate 4a, substituting 4,6-dichloro-2-(2-methoxyethyl)pyrazolo[3,4-d]pyrimidine (Intermediate 123d) in place of 4,6-dichloro-3-methyl-1H-pyrazolo[3,4-d]pyrimidine. MS (m/z) 384.20 [M+H]+. ¹H NMR (400 MHz, Acetonitrile-d3) δ 8.30 (s, 1H), 7.48 (d, J=16.7 Hz, 1H), 7.39 (s, 2H), 6.09 (d, J=16.7 Hz, 1H), 4.57 (t, J=5.0 Hz, 2H), 3.87 (t, J=5.0 Hz, 2H), 3.30 (s, 3H), 2.13 (s, 6H).

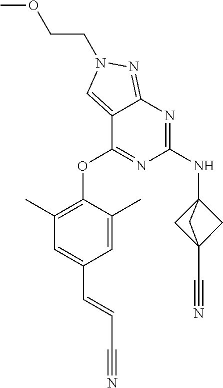 [SMILES: COCCn1cc2c(Oc3c(C)cc(/C=C/C#N)cc3C)nc(NC34CC(C#N)(C3)C4)nc2n1]

3-[[4-[4-[(E)-2-cyanovinyl]-2,6-dimethyl-phenoxy]-2-(2-methoxyethyl)pyrazolo[3,4-d]pyrimidin-6-yl]amino]bicyclo[1.1.1]pentane-1-carbonitrile—Compound 123 was prepared in a manner similar to Compound 4, substituting Intermediate 124e for Intermediate 4a. MS (m/z) 456.34 [M+H]+. ¹H NMR (400 MHz, Acetonitrile-d3) δ 8.04 (s, 1H), 7.48 (d, J=16.7 Hz, 1H), 7.38 (s, 2H), 6.11-6.00 (m, 2H), 4.41 (t, J=5.1 Hz, 2H), 3.83 (t, J=5.1 Hz, 2H), 3.29 (s, 3H), 2.13 (s, 6H), 2.12 (s, 6H).

### Example 124

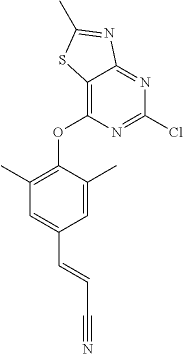 [SMILES: Cc1nc2nc(Cl)nc(Oc3c(C)cc(/C=C/C#N)cc3C)c2s1]

### Intermediate 124a(E)-3-[4-(5-chloro-2-methyl-thiazolo[4,5-d]pyrimidin-7-yl)oxy-3,5-dimethyl-phenyl]prop-2-enenitrile (Intermediate 124a)

5,7-dichloro-2-methyl-thiazolo[4,5-d]pyrimidine (100 mg, 0.454 mmol), (E)-3-(4-hydroxy-3,5-dimethyl-phenyl)prop-2-enenitrile (79 mg, 0.454 mmol), and potassium carbonate (75 mg, 0.545 mmol) were mixed in DMF (2.2 mL). The mixture was stirred at room temperature for 18 hours. The mixture was diluted with ethyl acetate and washed with water, then brine. The combined aqueous phases were extracted with more ethyl acetate. The combined organic phases were dried over magnesium sulfate, filtered, and concentrated in vacuo. Silica gel flash column chromatography (ethyl acetate/hexane) afforded the title compound. MS (m/z) 357.08 [M+H]+. ¹H NMR (400 MHz, Chloroform-d) δ 7.37 (d, J=16.7 Hz, 1H), 7.24 (s, 2H), 5.87 (d, J=16.6 Hz, 1H), 2.95 (s, 3H), 2.15 (s, 6H).

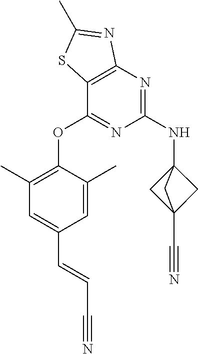 [SMILES: Cc1nc2nc(NC34CC(C#N)(C3)C4)nc(Oc3c(C)cc(/C=C/C#N)cc3C)c2s1]

3-[[7-[4-[(E)-2-cyanovinyl]-2,6-dimethyl-phenoxy]-2-methyl-thiazolo[4,5-d]pyrimidin-5-yl]amino]bicyclo[11.1.1]pentane-1-carbonitrile—Compound 124 was prepared in a manner similar to Compound 119, substituting Intermediate 124a Intermediate 124a in place of Intermediate 119a and stirring at 145° C. for 12 hr. MS (m/z) 429.23 [M+H]+. ¹H NMR (400 MHz, Acetonitrile-d3) δ 7.48 (d, J=16.7 Hz, 1H), 7.39 (s, 2H), 6.34 (s, 1H), 6.07 (d, J=16.7 Hz, 1H), 2.82 (s, 3H), 2.13 (s, 6H), 2.11 (s, 6H).

### Example 125

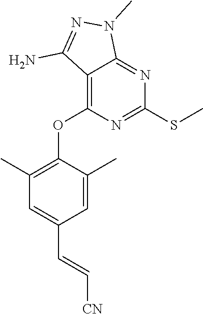

### (E)-3-(4-((3-amino-1-methyl-6-(methylthio)-1H-pyrazolo[3,4-d]pyrimidin-4-yl)oxy)-3,5-dimethylphenyl)acrylonitrile (Intermediate 125a)

4-chloro-1-methyl-6-methylsulfanyl-pyrazolo[3,4-d]pyrimidin-3-amine (100 mg, 0.435 mmol), (E)-3-(4-hydroxy-3,5-dimethyl-phenyl)prop-2-enenitrile (75 mg, 0.435 mmol), and potassium carbonate (60 mg, 0.435 mmol) were mixed in DMF (1.80 mL). The mixture was stirred at room temperature for 24 hours. Water was added to the mixture. The resulting solid was collected by filtration to afford the title compound. MS (m/z) 367.43 [M+H]+. ¹H NMR (400 MHz, DMSO-d6) δ 7.61 (d, J=16.8 Hz, 1H), 7.48 (s, 2H), 6.43 (d, J=16.7 Hz, 1H), 5.79 (s, 2H), 3.68 (s, 3H), 2.33 (s, 3H), 2.09 (s, 6H).

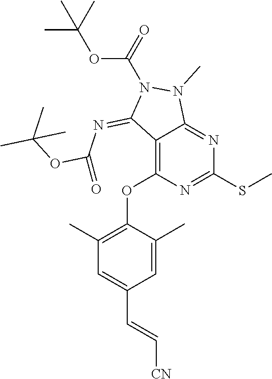 [SMILES: CSc1nc(Oc2c(C)cc(/C=C/C#N)cc2C)c2/c(=N\C(=O)OC(C)(C)C)n(C(=O)OC(C)(C)C)n(C)c2n1]

### tert-butyl (E)-3-((tert-butoxycarbonyl)imino)-4-(4-((E)-2-cyanovinyl)-2,6-dimethylphenoxy)-1-methyl-6-(methylthio)-1,3-dihydro-2H-pyrazolo[3,4-d]pyrimidine-2-carboxylate (Intermediate 125b)

Intermediate 126a (25 mg, 0.0682 mmol), 4-dimethylaminopyridine (0.8 mg, 0.00682 mmol), di-tert-butyl dicarbonate (22.3 mg, 0.102 mmol) were mixed with acetonitrile (0.68 mL) under argon, then Et₃N (0.050 mL, 0.341 mmol) was added. The mixture sealed under argon and stirred at 20° C. for 2 hr. The mixture was partitioned between saturated aqueous NH₄Cl and EtOAc. The organic phase was washed once more with saturated aqueous NH₄Cl, then water, then brine. The organic phase was dried over MgSO₄, filtered, and concentrated in vacuo. Silica gel flash column chromatography (0 to 100% EtOAc in hexane) afforded the title compound. MS (m/z) 566.75 [M+H]+. ¹H NMR (400 MHz, Chloroform-d) δ 7.35 (d, J=16.7 Hz, 1H), 7.19 (s, 2H), 5.84 (d, J=16.6 Hz, 1H), 4.03 (s, 3H), 2.34 (s, 3H), 2.11 (s, 6H), 1.47-1.41 (m, 18H).

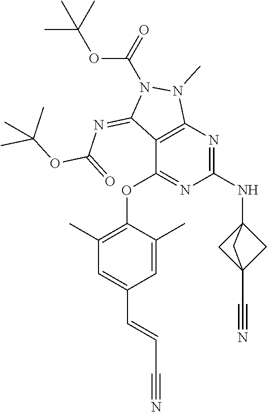 [SMILES: Cc1cc(/C=C/C#N)cc(C)c1Oc1nc(NC23CC(C#N)(C2)C3)nc2c1/c(=N\C(=O)OC(C)(C)C)n(C(=O)OC(C)(C)C)n2C]

### tert-butyl (3E)-3-tert-butoxycarbonylamino-6-[(3-cyano-1-bicyclo[1.1.1]pentanyl)amino]-4-[4-[(E)-2-cyanovinyl]-2,6-dimethyl-phenoxy]-1-methyl-pyrazolo[3,4-d]pyrimidine-2-carboxylate (Intermediate 125c)

Intermediate 125b (20 mg, 0.0353 mmol) and 3-chloroperoxybenzoic acid (20 mg, 0.088 mmol) were mixed with NMP (0.35 mL). The mixture was sealed under ambient atmosphere and stirred at 20° C. for 60 min. Then more mCPBA (4.4 mg, 0.018 mmol) was added and the mixture was stirred at 20° C. for another 15 min. Then potassium phosphate (45 mg, 0.212 mmol) and 3-aminobicyclo[1.1.1]pentane-1-carbonitrile (15.3 mg, 0.141 mmol) were added. The mixture was stirred in metal heating block at 50° C. for 2 hr. The mixture was diluted with DMF and filtered to remove insoluble, then was purified directly by preparative HPLC (30 to 100% MeCN in water, 0.1% TFA modifier). Fractions containing product were collected, partitioned between water and EtOAc, and treated with 1.1 equivalents NaHCO₃ relative to the amount of TFA present in the fraction volume. The resulting organic phase was washed with additional water, then dried over MgSO₄, filtered, and concentrated in vacuo to afford the title compound. MS (m/z) 627.04 [M+H]+.

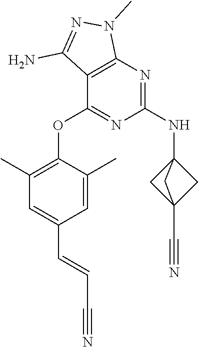 [SMILES: Cc1cc(/C=C/C#N)cc(C)c1Oc1nc(NC23CC(C#N)(C2)C3)nc2c1c(N)nn2C]

### 3-[[3-amino-4-[4-[(E)-2-cyanovinyl]-2,6-dimethyl-phenoxy]-1-methyl-pyrazolo[3,4-d]pyrimidin-6-yl]amino]bicyclo[1.1.1]pentane-1-carbonitrile—Compound 125

Intermediate 125c (2.8 mg) was mixed with trifluoroacetic acid (0.1 mL) and stirred at 20° C. for 30 min, then diluted with acetonitrile and concentrated in vacuo. The mixture was purified by preparative HPLC (30 to 100% MeCN in water, 0.1% TFA modifier). Fractions containing product were collected, partitioned between water and EtOAc, and treated with 1.1 equivalents NaHCO₃ relative to the amount of TFA present in the fraction volume. The resulting organic phase was washed with additional water, then dried over MgSO₄, filtered, and concentrated in vacuo to afford the title compound. MS (m/z) 427.27 [M+H]+. ¹H NMR (400 MHz, Acetonitrile-d3) δ 7.47 (d, J=16.7 Hz, 1H), 7.37 (s, 2H), 6.12 (s, 1H), 6.05 (d, J=16.7 Hz, 1H), 4.69 (s, 2H), 3.58 (s, 3H), 2.15-2.11 (m, 12H).

### Example 126

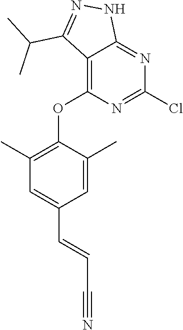 [SMILES: Cc1cc(/C=C/C#N)cc(C)c1Oc1nc(Cl)nc2[nH]nc(C(C)C)c12]

(E)-3-[4-[(6-chloro-3-isopropyl-1H-pyrazolo[3,4-d]pyrimidin-4-yl)oxy]-3,5-dimethyl-phenyl]prop-2-enenitrile (Intermediate 126a) was prepared in a manner similar to the intermediate for Compound 119, substituting 4,6-dichloro-3-isopropyl-1H-pyrazolo[3,4-d]pyrimidine for 4,6-dichloro-3-methyl-1H-pyrazolo[3,4-d]pyrimidine, and purifying by reverse-phase preparative HPLC (30 to 100% MeCN in water, 0.1% TFA modifier), collecting fractions containing the title compound and partitioning between water and ethyl acetate, treating with 1.1 equivalents of solid NaHCO₃ relative to TFA present in fractions, washing the resulting organic phase with more water, and concentrating in vacuo. MS (m/z) 368.13 [M+H]+. ¹H NMR (400 MHz, Acetonitrile-d3) δ 11.47 (s, 1H), 7.48 (d, J=16.7 Hz, 1H), 7.39 (s, 2H), 6.08 (d, J=16.7 Hz, 1H), 3.51 (hept, J=7.0 Hz, 1H), 2.13 (s, 6H), 1.45 (s, 3H), 1.43 (s, 3H).

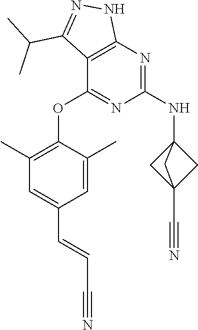 [SMILES: Cc1cc(/C=C/C#N)cc(C)c1Oc1nc(NC23CC(C#N)(C2)C3)nc2[nH]nc(C(C)C)c12]

### 3-[[4-[4-[(E)-2-cyanovinyl]-2,6-dimethyl-phenoxy]-3-isopropyl-1H-pyrazolo[3,4-d]pyrimidin-6-yl]amino]bicyclo[1.1.1]pentane-1-carbonitrile—Compound 126

3-aminobicyclo[1.1.1]pentane-1-carbonitrile (16.5 mg, 0.15 mmol), Intermediate 126a (11.5 mg, 0.0313 mmol) and diisopropylethylamine (0.020 mL, 0.125 mmol) were mixed with in NMP (0.5 mL). The mixture was stirred under nitrogen at 60° C. for 48 hr. Then more 3-aminobicyclo[1.1.1]pentane-1-carbonitrile (14 mg, 0.13 mmol) was added. The mixture was stirred under nitrogen at 60° C. for another 48 hr. The mixture was diluted with DMF, filtered, and purified via preparative HPLC (0-100% MeCN in water) then lyophilized to afford the title compound. MS (m/z) 440.22 [M+H]+. ¹H NMR (400 MHz, Acetonitrile-d3) δ 7.51 (d, J=16.7 Hz, 1H), 7.43 (s, 2H), 6.23 (s, 1H), 6.09 (d, J=16.7 Hz, 1H), 3.46-3.36 (m, 1H), 2.16-2.07 (m, 12H), 1.44 (d, J=6.9 Hz, 6H).

### Example 127

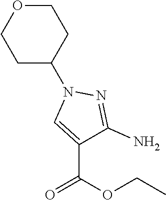 [SMILES: CCOC(=O)c1cn(C2CCOCC2)nc1N]

### ethyl 3-amino-1-(tetrahydro-2H-pyran-4-yl)-1H-pyrazole-4-carboxylate (Intermediate 127a)

Oxan-4-ylhydrazine hydrochloride (15.0 g, 98.2 mmol) and anisaldehyde (13.4 g, 98.2 mmol) in THE (300 mL) was stirred for 16 h at 70° C. The mixture was cooled to room temperature and concentrated under reduced pressure. The residue was dissolved in toluene (300 mL), to the above mixture was added DIEA (12.7 g, 98.2 mmol, 1.00 equiv) and ethyl (2E)-2-cyano-3-ethoxyprop-2-enoate (16.6 g, 98.2 mmol). The mixture was warmed to 110° C. and stirred for 16 h. The mixture was cooled to room temperature and concentrated under reduced pressure. The residue was purified by silica gel flash column chromatography (DCM/MeOH) to afford a residue to which was added EtOH (200 mL), then concentrated HCl (7.4 mL). The resulting mixture was stirred for 16 h at 80° C. The mixture was cooled to room temperature and concentrated under reduced pressure. The residue was purified by silica gel flash column chromatography (DCM/MeOH) to afford the title compound. MS: (m/z): 240.30 [M+H]+

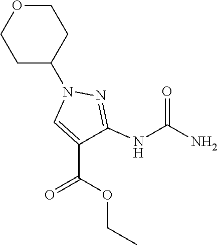 [SMILES: CCOC(=O)c1cn(C2CCOCC2)nc1NC(N)=O]

### ethyl 1-(tetrahydro-2H-pyran-4-yl)-3-ureido-1H-pyrazole-4-carboxylate (Intermediate 127b)

To ethyl 3-amino-1-(oxan-4-yl)pyrazole-4-carboxylate (8.40 g, 35.1 mmol) in AcOH (50 mL) was added potassium cyanate (5.70 g, 70.2 mmol). The resulting mixture was stirred for 4 h at room temperature. The mixture was concentrated and the residue was adjusted to pH 8 with saturated aqueous NaHCO₃ and extracted with EtOAc. The combined organic layers were washed with brine, dried over anhydrous sodium sulfate. The organic layers were concentrated and the residue was purified by silica gel flash column chromatography (DCM/MeOH) to afford the title compound. MS: (m/z): 283.10 [M+H]+

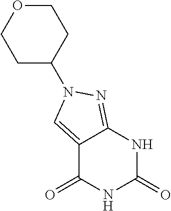 [SMILES: O=c1[nH]c(=O)c2cn(C3CCOCC3)nc2[nH]1]

### 2-(tetrahydro-2H-pyran-4-yl)-2,7-dihydro-4H-pyrazolo[3,4-d]pyrimidine-4,6(5H)-dione (Intermediate 127c)

To ethyl 1-(tetrahydro-2H-pyran-4-yl)-3-ureido-1H-pyrazole-4-carboxylate (5.00 g, 17.7 mmol, 1.00 equiv) in EtOH (100 mL) was added EtONa (2.41 g, 35.4 mmol, 2.00 equiv) at 25° C. Then the mixture was stirred for 1 h at 80° C. The mixture was cooled to room temperature and concentrated under reduced pressure. The residue was dissolved with water, acidified to pH 6 with HCl (2M in water) at 0° C. and extracted with EtOAc. The combined organic layers were washed with brine and dried over anhydrous sodium sulfate. The organic layers were concentrated and the residue was purified by silica gel flash column chromatography (DCM/MeOH) to afford the title compound as a white solid. MS: (m/z): 237.10 [M+H]+

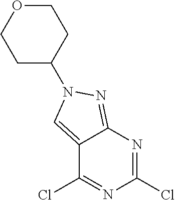 [SMILES: Clc1nc(Cl)c2cn(C3CCOCC3)nc2n1]

### 4,6-dichloro-2-(tetrahydro-2H-pyran-4-yl)-2H-pyrazolo[3,4-d]pyrimidine (Intermediate 127d)

A mixture of 2-(oxan-4-yl)-5H,7H-pyrazolo[3,4-d]pyrimidine-4,6-dione (2.70 g, 11.4 mmol) in POCl₃ (30 mL) was stirred for 16 at 100° C. The mixture was cooled to room temperature and concentrated under reduced pressure. The residue was dissolved with EtOAc, treated with ice water and extracted with more EtOAc. The combined organic phases were washed with brine, dried over anhydrous sodium sulfate. The organic phases were concentrated and the residue was purified by silica gel flash column chromatography (DCM/EtOAc) to afford the title compound. ¹H NMR (400 MHz, Chloroform-d) δ 8.22 (s, 1H), 4.76-4.65 (m, 1H), 4.19 (dt, J=11.9, 3.4 Hz, 2H), 3.68-3.56 (m, 2H), 2.33-2.21 (m, 4H). LCMS: (m/z): 272.95, [M+H]+.

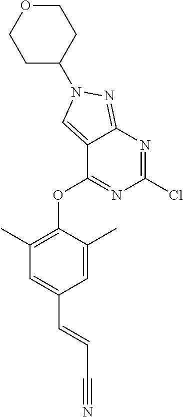 [SMILES: Cc1cc(/C=C/C#N)cc(C)c1Oc1nc(Cl)nc2nn(C3CCOCC3)cc12]

### (E)-3-(4-((6-chloro-2-(tetrahydro-2H-pyran-4-yl)-2H-pyrazolo[3,4-d]pyrimidin-4-yl)oxy)-3,5-dimethylphenyl)acrylonitrile (Intermediate 127e)

The title compound was prepared in a manner similar to Intermediate 4a, substituting 4,6-dichloro-2-tetrahydropyran-4-yl-pyrazolo[3,4-d]pyrimidine in place of 4,6-dichloro-3-methyl-1H-pyrazolo[3,4-d]pyrimidine. MS (m/z) 410.18 [M+H]+. ¹H NMR (400 MHz, Acetonitrile-d3) δ 8.38 (s, 1H), 7.48 (d, J=16.7 Hz, 1H), 7.39 (s, 2H), 6.09 (d, J=16.7 Hz, 1H), 4.79-4.62 (m, 1H), 4.14-3.96 (m, 2H), 3.66-3.43 (m, 2H), 2.18-2.14 (m, 4H), 2.13 (s, 6H).

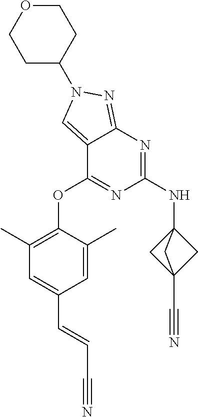 [SMILES: Cc1cc(/C=C/C#N)cc(C)c1Oc1nc(NC23CC(C#N)(C2)C3)nc2nn(C3CCOCC3)cc12]

### 3-[[4-[4-[(E)-2-cyanovinyl]-2,6-dimethyl-phenoxy]-2-tetrahydropyran-4-yl-pyrazolo[3,4-d]pyrimidin-6-yl]amino]bicyclo[11.1.1]pentane-1-carbonitrile—Compound 127

The title compound was prepared in a manner similar to Compound 4, substituting Intermediate 127e for Intermediate 4a. MS (m/z) 482.33 [M+H]+. ¹H NMR (400 MHz, Acetonitrile-d3) δ 8.11 (d, J=5.1 Hz, 1H), 7.48 (d, J=16.7 Hz, 1H), 7.38 (s, 2H), 6.10-5.94 (m, 2H), 4.55 (dd, J=10.4, 5.0 Hz, 1H), 4.12-3.95 (m, 2H), 3.60-3.43 (m, 2H), 2.35-2.20 (m, 4H), 2.12-2.07 (m, 12H).

### Example 128

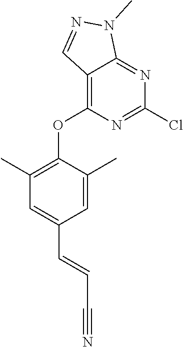 [SMILES: Cc1cc(/C=C/C#N)cc(C)c1Oc1nc(Cl)nc2c1cnn2C]

(E)-3-[4-(6-chloro-1-methyl-pyrazolo[3,4-d]pyrimidin-4-yl)oxy-3,5-dimethyl-phenyl]prop-2-enenitrile (Intermediate 128a) was prepared in a manner similar to Intermediate 121a, substituting 4,6-dichloro-1-methyl-pyrazolo[3,4-d]pyrimidine for 4,6-dichloro-2-methyl-pyrazolo[3,4-d]pyrimidine. MS (m/z) 340.11 [M+H]+. ¹H NMR (400 MHz, DMSO-d6) δ 8.15 (s, 1H), 7.64 (d, J=16.7 Hz, 1H), 7.54 (s, 2H), 6.47 (d, J=16.7 Hz, 1H), 4.01 (s, 3H), 2.09 (s, 6H).

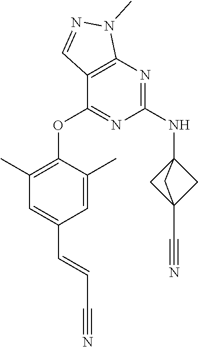 [SMILES: Cc1cc(/C=C/C#N)cc(C)c1Oc1nc(NC23CC(C#N)(C2)C3)nc2c1cnn2C]

### 3-[[4-[4-[(E)-2-cyanovinyl]-2,6-dimethyl-phenoxy]-1-methyl-pyrazolo[3,4-d]pyrimidin-6-yl]amino]bicyclo[11.1.1]pentane-1-carbonitrile—Compound 128

Intermediate 128a (50 mg, 0.147 mmol), 3-aminobicyclo[1.1.1]pentane-1-carbonitrile (48 mg, 0.441 mmol), rac BINAP Pd G3 (14.6 mg, 0.015 mmol), and cesium carbonate (144 mg, 0.441 mmol) were mixed with dioxane (1.1 mL). The mixture was degassed with N₂ and then the vial sealed and heated to 120° C. for 30 min. The mixture was then cooled to room temperature, diluted with ethyl acetate, filtered and concentrated in vacuo. Purification by preparative HPLC (0-100% MeCN/water) afforded the title compound. MS (m/z) 412.12 [M+H]+. ¹H NMR (400 MHz, Acetonitrile-d3) δ 7.80 (s, 1H), 7.51 (d, J=16.7 Hz, 1H), 7.41 (s, 2H), 6.33 (s, 1H), 6.09 (d, J=16.7 Hz, 1H), 3.86 (s, 3H), 2.20-2.15 (m, 12H).

### Example 129

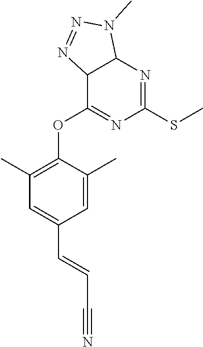

(E)-3-[3,5-dimethyl-4-(3-methyl-5-methylthio-triazolo[4,5-d]pyrimidin-7-yl)oxy-phenyl]prop-2-enenitrile (Intermediate 129a) was prepared in a manner similar to Intermediate 120a, substituting 7-chloro-3-methyl-5-methylthio-triazolo[4,5-d]pyrimidine in place of 4,6-dichloro-3-methyl-1H-pyrazolo[3,4-d]pyrimidine. MS (m/z) 353.09 [M+H]+. ¹H NMR (400 MHz, DMSO-d6) δ 7.63 (d, J=16.7 Hz, 1H), 7.54 (s, 2H), 6.46 (d, J=16.7 Hz, 1H), 4.21 (s, 3H), 2.43 (s, 3H), 2.10 (s, 6H).

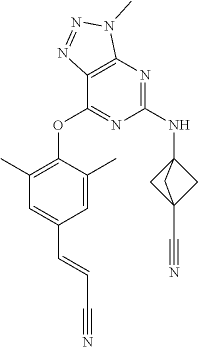 [SMILES: Cc1cc(/C=C/C#N)cc(C)c1Oc1nc(NC23CC(C#N)(C2)C3)nc2c1nnn2C]

### 3-[[7-[4-[(E)-2-cyanovinyl]-2,6-dimethyl-phenoxy]-3-methyl-triazolo[4,5-d]pyrimidin-5-yl]amino]bicyclo[11.1.1]pentane-1-carbonitrile—Compound 1129

Intermediate 129a (25 mg, 0.0709 mmol) and 3-chloroperoxybenzoic acid (49 mg, 0.213 mmol) were mixed with NMP (0.71 mL). The mixture was sealed under ambient atmosphere and stirred at 20° C. for 60 min. Then potassium phosphate (90 mg, 0.426 mmol) and 3-aminobicyclo[1.1.1]pentane-1-carbonitrile hydrochloride (31 mg, 0.213 mmol) were added. The mixture was stirred 20° C. for 21 hr, then in metal heating block at 50° C. for 1 hr. The mixture was diluted with DMF and filtered to remove insoluble solids, then was purified directly by preparative HPLC (30 to 100% MeCN in water, 0.1% TFA modifier). Fractions containing product were collected, partitioned between water and EtOAc, and treated with 1.1 equivalents NaHCO₃ relative to the amount of TFA present in the fraction volume. The resulting organic phase was washed with additional water, then dried over MgSO₄, filtered, and concentrated in vacuo to afford the title compound. MS (m/z) 413.01 [M+H]+. ¹H NMR (400 MHz, Acetonitrile-d3) δ 7.52 (d, J=16.7 Hz, 1H), 7.44 (s, 2H), 6.58 (s, 1H), 6.11 (d, J=16.7 Hz, 1H), 4.06 (s, 3H), 2.19-2.14 (m, 12H).

### Example 130

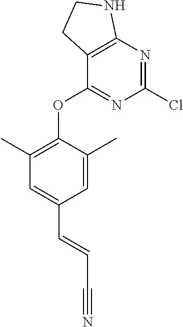 [SMILES: Cc1cc(/C=C/C#N)cc(C)c1Oc1nc(Cl)nc2c1CCN2]

### (E)-3-[4-[(2-chloro-6,7-dihydro-5H-pyrrolo[2,3-d]pyrimidin-4-yl)oxy]-3,5-dimethyl-phenyl]prop-2-enenitrile (Intermediate 130a)

2,4-dichloro-6,7-dihydro-5H-pyrrolo[2,3-d]pyrimidine (0.22 g, 1.16 mmol), (E)-3-(4-hydroxy-3,5-dimethyl-phenyl)prop-2-enenitrile (201 mg, 1.16 mmol), and potassium carbonate (160 mg, 1.16 mmol) were mixed in DMF (5.6 mL). The mixture was stirred at 90° C. for 6 hours. The mixture was partitioned between EtOAc and saturated aqueous NH₄Cl, then the organic phase was washed with water, then brine. The organic phase was dried over magnesium sulfate, filtered, and concentrated in vacuo to afford the title compound. MS (m/z) 327.16 [M+H]+.

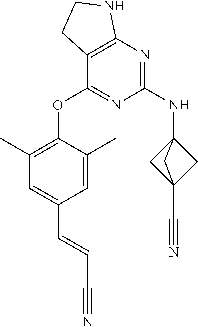 [SMILES: Cc1cc(/C=C/C#N)cc(C)c1Oc1nc(NC23CC(C#N)(C2)C3)nc2c1CCN2]

3-[[4-[4-[(E)-2-cyanovinyl]-2,6-dimethyl-phenoxy]-6,7-dihydro-5H-pyrrolo[2,3-d]pyrimidin-2-yl]amino]bicyclo[11.1.1]pentane-1-carbonitrile—Compound 130 was prepared in a manner similar to Compound 128, substituting Intermediate 130a for Intermediate 128a. MS (m/z) 399.22 [M+H]+. ¹H NMR (400 MHz, Acetonitrile-d3) δ 7.45 (d, J=16.6 Hz, 1H), 7.33 (s, 2H), 6.02 (d, J=16.7 Hz, 1H), 5.71 (s, 1H), 5.21 (s, 1H), 3.61-3.53 (m, 2H), 2.95 (t, J=8.6 Hz, 2H), 2.11 (s, 6H), 2.06-2.00 (m, 3H). [additional alkyl C—H signal likely buried under acetonitrile solvent peak].

### Example 131 and 132

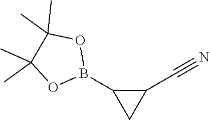

### 2-(4,4,5,5-tetramethyl-1,3,2-dioxaborolan-2-yl)cyclopropane-1-carbonitrile (Intermediate 131a)

To cyclopropanecarbonitrile (200 g, 2980 mmol) and bis(pinacolato)diboron (681 g, 2680 mmol) was added THE (2000 mL), then [Ir(COD)OMe]₂ (19.7 g, 29.8 mmol) and 2,9-dimethyl-1,10-phenanthroline (18.6 g, 89.4 mmol, 0.03 eq). The mixture was degassed and purged with nitrogen, then heated with stirring at 90° C. for 12 hr, with monitoring by silica gel TLC (Petroleum ether/Ethyl acetate). The mixture was cooled to 25° C., filtered and concentrated in vacuum. Purification by silica gel chromatography (Petroleum ether/Ethyl acetate) afforded the title compound as a mixture of stereoisomers. ¹H NMR (CDCl₃ 400 MHz): δ 1.51-1.46 (m, 1H), 1.32-1.27 (m, 2H), 1.22 (s, 11H), 1.11-1.06 (m, 1H), 0.65-0.59 (m, 1H).

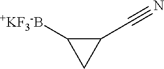

### Potassium (2-cyanocyclopropyl)trifluoroborate (Intermediate 131b)

To 2-(4,4,5,5-tetramethyl-1,3,2-dioxaborolan-2-yl)cyclopropane-1-carbonitrile (33.0 g, 171 mmol) was added MeOH (330 mL) and H₂O (82.5 mL), then KHF₂ (66.7 g, 855 mmol). The mixture was stirred at 25° C. for 12 hr, with reaction monitoring by silica gel TLC (Petroleum ether/Ethyl acetate). The mixture was cooled to 25° C., filtered and concentrated in vacuum. The resulting residue was poured into acetone, dried with anhydrous Na₂SO₄, filtered and the filtrate concentrated in vacuo. Trituration with petroleum ether/ethyl acetate at 25° C. for 1 hr afforded the title compound as a mixture of stereoisomers. ¹H NMR (DMSO-d₆400 MHz): δ 0.86-0.83 (m, 1H), 0.74 (d, J=10 Hz, 1H), 0.58-0.55 (s, 1H), 0.02-0.03 (m, 1H).

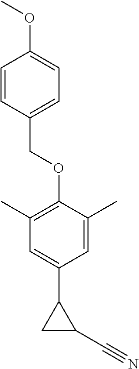 [SMILES: COc1ccc(COc2c(C)cc(C3CC3C#N)cc2C)cc1]

### 2-(4-((4-methoxybenzyl)oxy)-3,5-dimethylphenyl)cyclopropane-1-carbonitrile (Intermediate 131c)

To potassium (2-cyanocyclopropyl)trifluoroborate (41.9 g, 130 mmol) and 5-bromo-2-((4-methoxybenzyl)oxy)-1,3-dimethylbenzene (18.8 g, 109 mmol) in dioxane (376 mL) and H₂O (94.0 mL) was added Cs₂CO₃ (106 g, 326 mmol) and [di(adamantan-1-yl)(butyl)phosphine](methanesulfonato-κO)[2′-(methylamino)-2-biphenylyl]palladium (CataCXium A Pd G4 precatalyst, 6.74 g, 9.24 mmol). The mixture was degassed and purged with nitrogen, then heated with stirring at 100° C. for 12 hr, with monitoring by silica gel TLC (Petroleum ether/Ethyl acetate). The reaction mixture is poured into H₂O, the aqueous phase is extracted twice with ethyl acetate. The combined organic phase is washed twice with brine, dried with anhydrous Na₂SO₄, filtered and concentrated in vacuum. Purification by silica gel chromatography (Petroleum ether/Ethyl acetate) afforded the title compound as a mixture of stereoisomers. ¹H NMR (CDCl₃ 400 MHz): δ 7.40-7.37 (m, 2H), 6.96-6.93 (m, 2H), 6.78 (s, 2H), 4.72 (s, 2H), 3.84 (s, 3H), 2.58-2.52 (m, 1H), 2.28 (s, 6H), 1.60-1.55 (m, 1H), 1.53-1.48 (m, 1H), 1.44-1.39 (m, 1H).

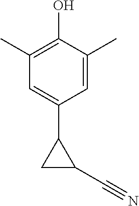

### 2-(4-hydroxy-3,5-dimethylphenyl)cyclopropane-1-carbonitrile (Intermediate 131d)

2-(4-((4-methoxybenzyl)oxy)-3,5-dimethylphenyl)cyclopropane-1-carbonitrile (16.9 g, 55.0 mmol) was stirred in trifluoroacetic acid (84.5 mL) and DCM (169 mL) at 25° C. for 1 h, with reaction monitoring by LCMS. The mixture was concentrated in vacuum. Purification by silica gel chromatography (Petroleum ether/Ethyl acetate) yielded a crude product. Pouring crude product into MTBE and adding petroleum ether dropwise, with stirring at 10° C. for 30 min, followed by collection of the resulting solid by filtration afforded the title compound as a mixture of stereoisomers. MS (m/z) 188.2 [M+H]+. ¹H NMR (MeOD-d₄ 400 MHz): δ 6.85 (s, 1H), 6.72 (s, 1H), 2.50-2.43 (m, 1H), 2.20-2.17 (m, 6H), 1.82-1.86 (m, 1H), 1.63-1.58 (m, 1H), 1.50-1.37 (m, 2H).

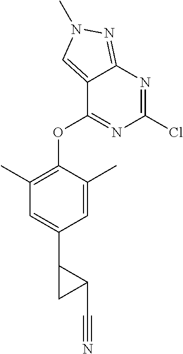 [SMILES: Cc1cc(C2CC2C#N)cc(C)c1Oc1nc(Cl)nc2nn(C)cc12]

2-(4-((6-chloro-2-methyl-2H-pyrazolo[3,4-d]pyrimidin-4-yl)oxy)-3,5-dimethylphenyl)cyclopropane-1-carbonitrile (Intermediate 131e) was prepared as a mixture of stereoisomers in a manner similar to Intermediate 124a substituting Intermediate 131d for 5,7-dichloro-2-methyl-thiazolo[4,5-d]pyrimidine. MS (m/z) 354.28 [M+H]+. (rac)-3-[[4-[4-[(1R,2S)-2-cyanocyclopropyl]-2,6-dimethyl-phenoxy]-2-methyl-pyrazolo[3,4-d]pyrimidin-6-yl]amino]bicyclo[1.1.1]pentane-1-carbonitrile were synthesized together using a procedure similar to Compound 120, substituting Intermediate 131e for Intermediate 121a. Following preparative reverse-phase HPLC (30 to 90% MeCN in water, 0.1% trifluoroacetic acid modifier), Compound 132 was obtained as the earlier eluting diastereomer, and Compound 131 was obtained as the later-eluting diastereomer.

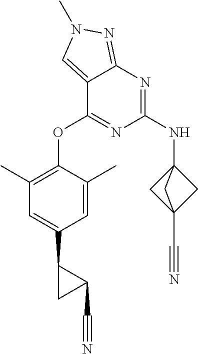 [SMILES: Cc1cc([C@@H]2C[C@@H]2C#N)cc(C)c1Oc1nc(NC23CC(C#N)(C2)C3)nc2nn(C)cc12]

### the Cis, Racemic, First Diastereomer was Eluted by Reverse-Phase HPLC

Compound 131 MS (m/z) 426.30 [M+H]+. ¹H NMR (400 MHz, Acetonitrile-d3) δ 7.63 (s, 1H), 7.13 (s, 2H), 6.12 (s, 1H), 3.96 (s, 3H), 2.56 (q, J=8.0 Hz, 1H), 2.15 (s, 6H), 2.09 (s, 6H), 2.00-1.96 (m, 1H), 1.57 (dt, J=7.5, 5.8 Hz, 1H), 1.54-1.47 (m, 1H).

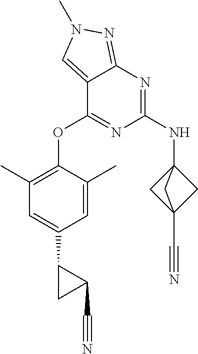 [SMILES: Cc1cc([C@H]2C[C@@H]2C#N)cc(C)c1Oc1nc(NC23CC(C#N)(C2)C3)nc2nn(C)cc12]

### the Trans, Racemic, Second Diastereomer was Eluted by Reverse-Phase HPLC

Compound 132 (rac)-3-((4-(4-((1S,2S)-2-cyanocyclopropyl)-2,6-dimethylphenoxy)-2-methyl-2H-pyrazolo[3,4-d]pyrimidin-6-yl)amino)bicyclo[1.1.1]pentane-1-carbonitrile MS (m/z) 426.26 [M+H]+. ¹H NMR (400 MHz, Acetonitrile-d3) δ 7.94 (s, 1H), 6.99 (s, 2H), 6.06 (s, 1H), 4.01 (s, 3H), 2.62 (ddd, J=9.3, 6.7, 4.8 Hz, 1H), 2.13 (s, 6H), 2.06 (s, 6H), 1.70 (ddd, J=8.9, 5.6, 4.7 Hz, 1H), 1.59 (dt, J=9.2, 5.4 Hz, 1H), 1.46 (ddd, J=9.0, 6.7, 5.3 Hz, 1H).

### Example 133 and 134

### 3-((4-(4-((1S,2S)-2-cyanocyclopropyl)-2,6-dimethylphenoxy)-2-methyl-2H-pyrazolo[3,4-d]pyrimidin-6-yl)amino)bicyclo[1.1.1]pentane-1-carbonitrile—Compound 133

### (rac)-3-((4-(4-((1R,2R)-2-cyanocyclopropyl)-2,6-dimethylphenoxy)-2-methyl-2H-pyrazolo[3,4-d]pyrimidin-6-yl)amino)bicyclo[1.1.1]pentane-1-carbonitrile—Compound 134

Preparative chiral SFC (OJ-H column, 30% MeOH in CO₂) on Compound 13 afforded the title compounds, as the first enantiomer to elute (Peak 1), and Compound 134 as the second enantiomer to elute (Peak 2). The absolute configuration has not been assigned for Compound 133 and Compound 134.

### Compound 133 (trans, Peak 1)

MS (m/z) 426.27 [M+H]+. ¹H NMR (400 MHz, Acetonitrile-d3) δ 7.93 (s, 1H), 6.99 (s, 2H), 6.05 (s, 1H), 4.01 (s, 3H), 2.61 (ddd, J=9.2, 6.7, 4.7 Hz, 1H), 2.14 (s, 6H), 2.06 (s, 6H), 1.70 (ddd, J=9.0, 5.6, 4.7 Hz, 1H), 1.59 (dt, J=9.2, 5.4 Hz, 1H), 1.46 (ddd, J=8.9, 6.7, 5.3 Hz, 1H).

### Compound 134 (trans, Peak 2)

MS (m/z) 426.27 [M+H]+. ¹H NMR (400 MHz, Acetonitrile-d3) δ 7.94 (s, 1H), 6.99 (s, 2H), 6.05 (s, 1H), 4.01 (s, 3H), 2.62 (ddd, J=9.2, 6.7, 4.7 Hz, 1H), 2.13 (s, 6H), 2.06 (s, 6H), 1.74-1.67 (m, 1H), 1.59 (dt, J=9.2, 5.4 Hz, 1H), 1.46 (ddd, J=8.9, 6.7, 5.3 Hz, 1H).

### Example 135

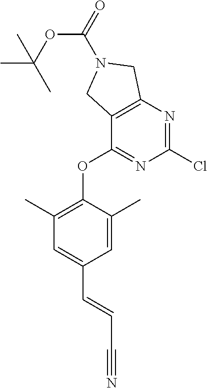 [SMILES: Cc1cc(/C=C/C#N)cc(C)c1Oc1nc(Cl)nc2c1CN(C(=O)OC(C)(C)C)C2]

tert-butyl (E)-2-chloro-4-(4-(2-cyanovinyl)-2,6-dimethylphenoxy)-5,7-dihydro-6H-pyrrolo[3,4-d]pyrimidine-6-carboxylate (Intermediate 135a) was prepared as a mixture of stereoisomers in a manner similar to Intermediate 124a substituting tert-butyl 2,4-dichloro-5,7-dihydropyrrolo[3,4-d]pyrimidine-6-carboxylate for 5,7-dichloro-2-methyl-thiazolo[4,5-d]pyrimidine. MS (m/z) 426.85 [M+H]+. ¹H NMR (400 MHz, Chloroform-d) δ 7.35 (d, J=16.6 Hz, 1H), 7.21 (d, J=1.9 Hz, 2H), 5.85 (d, J=16.6 Hz, 1H), 4.76-4.69 (m, 3H), 4.65 (t, J=2.5 Hz, 1H), 2.10-2.14 (m, 6H), 1.53 (s, 9H).

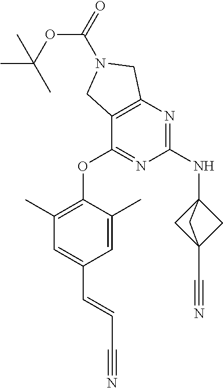 [SMILES: Cc1cc(/C=C/C#N)cc(C)c1Oc1nc(NC23CC(C#N)(C2)C3)nc2c1CN(C(=O)OC(C)(C)C)C2]

tert-butyl (E)-2-((3-cyanobicyclo[1.1.1]pentan-1-yl)amino)-4-(4-(2-cyanovinyl)-2,6-dimethylphenoxy)-5,7-dihydro-6H-pyrrolo[3,4-d]pyrimidine-6-carboxylate (Intermediate 135b) was prepared in a manner similar to Compound 119, substituting Intermediate 135a for Int-Compound 119 and stirring at 145° C. for 12 hr. MS (m/z) 499.03 [M+H]+.

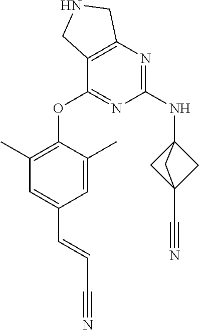 [SMILES: Cc1cc(/C=C/C#N)cc(C)c1Oc1nc(NC23CC(C#N)(C2)C3)nc2c1CNC2]

### (E)-3-((4-(4-(2-cyanovinyl)-2,6-dimethylphenoxy)-6,7-dihydro-5H-pyrrolo[3,4-d]pyrimidin-2-yl)amino)bicyclo[1.1.1]pentane-1-carbonitrile—Compound 135

Intermediate 135b (31 mg, 0.0622 mmol) was mixed with 4 N hydrogen chloride in 1,4-dioxane (0.16 mL, 0.622 mmol). After 5 min at 20° C., the mixture was treated with 2 M aqueous sodium carbonate, diluted with DMF, filtered, and purified by preparative reverse-phase HPLC (MeCN/water, no additional modifiers) to afford the title compound. MS (m/z) 399.17 [M+H]+. ¹H NMR (400 MHz, Acetonitrile-d3) δ 7.47 (d, J=16.7 Hz, 1H), 7.37 (s, 2H), 6.19 (s, 1H), 6.05 (d, J=16.7 Hz, 1H), 4.14 (s, 2H), 3.92 (t, J=1.9 Hz, 2H), 2.09 (s, 6H), 2.06 (s, 6H).

### Examples 136, 137, and 138

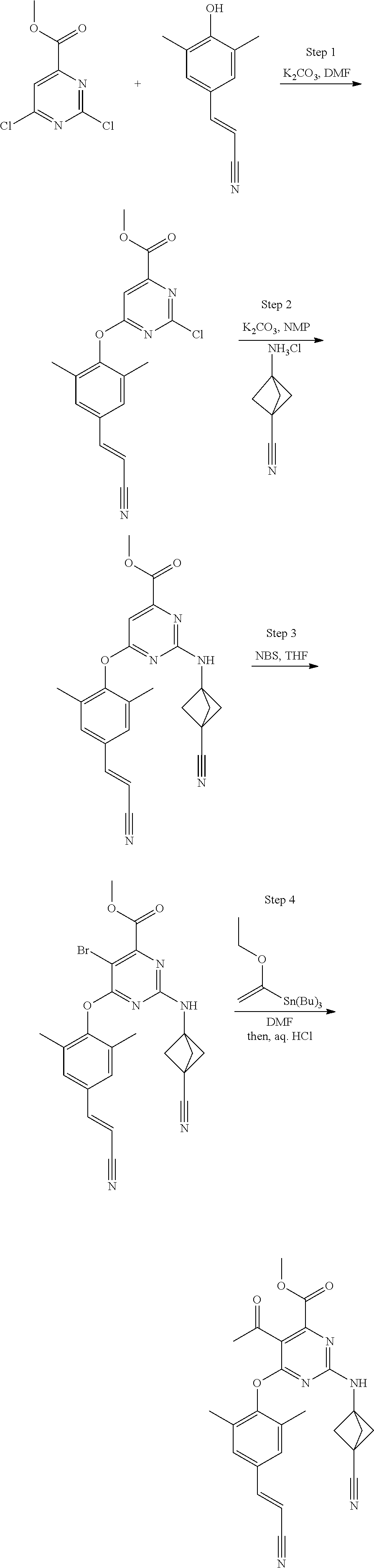

### Step 1, preparation of Methyl 2-chloro-6-[4-[(E)-2-cyanovinyl]-2,6-dimethyl-phenoxy]pyrimidine-4-carboxylate (Intermediate 136a)

(E)-3-(4-hydroxy-3,5-dimethyl-phenyl)prop-2-enenitrile (0.106 mol, 18.4 g), methyl 2,6-dichloropyrimidine-4-carboxylate (0.0966 mol, 20 g), and potassium carbonate (0.290 mol, 40.1 g) were suspended in DMF (200 mL). The mixture was stirred at room temperature for one hour at which point water was added to the mixture. The mixture was vigorously stirred and then subsequently filtered to yield the title compound which was used without further purification. MS (m/z) 344.1 [M+H]+. ¹H NMR (400 MHz, Acetonitrile-d3) δ 7.62 (s, 1H), 7.49 (d, J=16.7 Hz, 1H), 7.40 (s, 2H), 6.10 (d, J=16.7 Hz, 1H), 3.98 (s, 3H), 2.13 (s, 6H).

### Step 2, preparation of Methyl 2-[(3-cyano-1-bicyclo[1.1.1]pentanyl)amino]-6-[4-[(E)-2-cyanovinyl]-2,6-dimethyl-phenoxy]pyrimidine-4-carboxylate (Intermediate 136b)

Intermediate 136a (0.0960 mol, 33.0 g), 3-aminobicyclo[1.1.1]pentane-1-carbonitrile; hydrochloride (0.116 mol, 16.8 g), and potassium carbonate (0.480 mol, 66.3 g) were suspended in NMP (400 mL). The mixture was stirred at 60° C. overnight. The mixture was diluted with water at which point the solids were filtered off to yield the title compound which was used without further purification. MS (m/z) 416.2 [M+H]+.

### Step 3, preparation of Methyl 5-bromo-2-[(3-cyano-1-bicyclo[1.1.1]pentanyl)amino]-6-[4-[(E)-2-cyanovinyl]-2,6-dimethyl-phenoxy]pyrimidine-4-carboxylate (Intermediate 136c)

Intermediate 136b (0.0175 mol, 7.30 g) and N-bromosuccinimide (0.0193 mol, 3.44 g) were suspended in THE (100 mL). The mixture was stirred at room temperature for four hours. The mixture was diluted with water. The aqueous phase was extracted with EtOAc. The combined organic phase was washed with brine, dried over MgSO₄, filtered, and concentrated in vacuo to yield the title compound which was used without further purification. MS (m/z) 494.1 [M+H]+.

### Step 4, preparation of Methyl 5-acetyl-2-[(3-cyano-1-bicyclo[1.1.1]pentanyl)amino]-6-[4-[(E)-2-cyanovinyl]-2,6-dimethyl-phenoxy]pyrimidine-4-carboxylate (Intermediate 136d)

Intermediate 136c (0.101 mmol, 50.0 mg) and tetrakis(triphenylphosphine)palladium(0) (0.0202 mmol, 23.4 mg) were suspended in DMF (1 mL). Tributyl(1-ethoxyvinyl)stannane (0.202 mmol, 0.0683 mL) was added to the mixture. The mixture was stirred at 100° C. under an argon atmosphere overnight. Then, 1N aq. HCl (1 mL) was added to the mixture and was left to stir at RT for one hour. The mixture was quenched with 1N aq. NaHCO₃ (1 mL) and diluted with EtOAc. The aqueous phase was extracted twice with EtOAc. The combined organic phase was washed with brine, dried over MgSO₄, filtered, and concentrated in vacuo. The residue was purified via silica gel chromatography (0-100% EtOAc in hexanes) to afford the title compound (33.6 mg, 73% yield). MS (m/z) 458.1 [M+H]+. ¹H NMR (400 MHz, Acetonitrile-d3) δ 7.51 (d, J=16.7, 3.6 Hz, 1H), 7.43 (s, 2H), 6.10 (d, J=16.7, 2.0 Hz, 1H), 3.88 (s, 3H), 2.66 (s, 3H), 2.16 (s, 6H), 2.05 (s, 6H).

### Preparation of (E)-3-((4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile—Compound 136

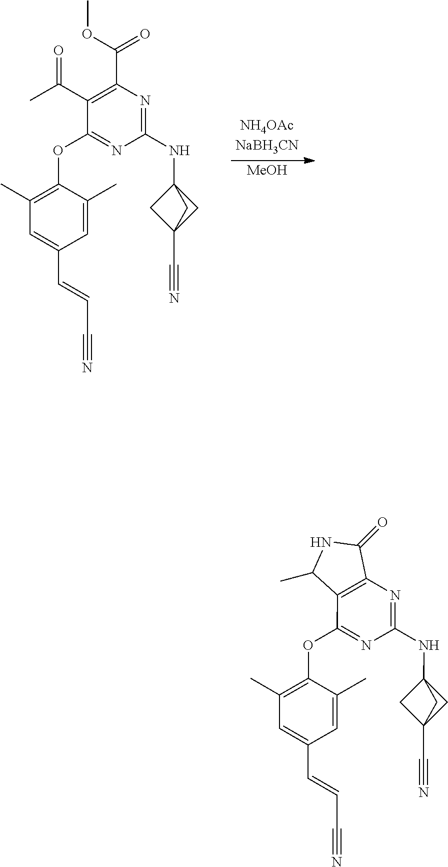 [SMILES: "MeOH" ; COC(=O)c1nc(NC23CC(C#N)(C2)C3)nc(Oc2c(C)cc(/C=C/C#N)cc2C)c1C(C)=O ; Cc1cc(/C=C/C#N)cc(C)c1Oc1nc(NC23CC(C#N)(C2)C3)nc2c1C(C)NC2=O]

Intermediate 136d (0.0328 mmol, 15 mg), ammonium acetate (0.492 mmol, 37.9 mg), and sodium cyanoborohydride (0.328 mmol, 20.6 mg) were suspended in MeOH (0.5 mL). The mixture was subject to microwave irradiation at 130° C. for 30 minutes. The mixture was then concentrated in vacuo, redissolved in DMF, filtered, and purified via preparative HPLC (0-100% MeCN in water, 0.1% TFA) then lyophilized to afford the title compound as the bis-trifluoroacetic acid salt (0.82 mg, 5.9% yield). MS (m/z) 427.2 [M+H]+. ¹H NMR (400 MHz, DMSO-d6) δ 9.24 (s, 1H), 8.54 (s, 1H), 7.64 (d, J=16.6 Hz, 1H), 7.57 (s, 2H), 6.44 (d, J=16.7 Hz, 1H), 4.75 (q, J=6.5 Hz, 1H), 2.15-1.86 (m, 12H), 1.48 (d, J=6.6 Hz, 3H).

### Preparation of 3-[[(5R)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[1.1.1]pentane-1-carbonitrile—Compound 137 and 3-[[(5S)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[1.1.1]pentane-1-carbonitrile—Compound 138

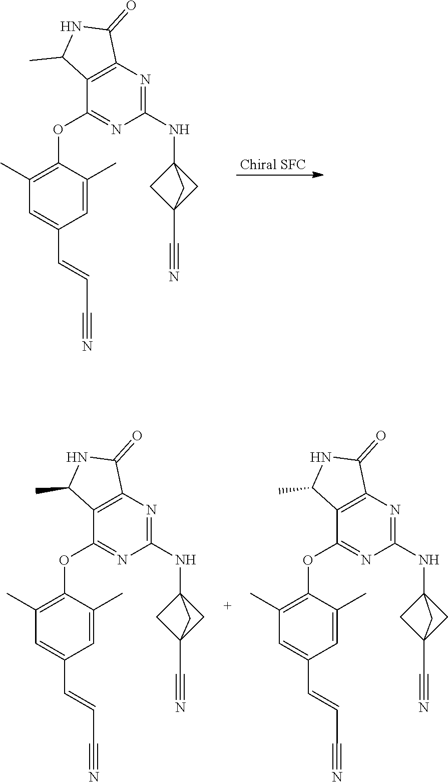 [SMILES: Cc1cc(/C=C/C#N)cc(C)c1Oc1nc(NC23CC(C#N)(C2)C3)nc2c1C(C)NC2=O ; "Chiral SFC" ; Cc1cc(/C=C/C#N)cc(C)c1Oc1nc(NC23CC(C#N)(C2)C3)nc2c1[C@@H](C)NC2=O ; Cc1cc(/C=C/C#N)cc(C)c1Oc1nc(NC23CC(C#N)(C2)C3)nc2c1[C@H](C)NC2=O]

Compound 136 (40 mg, 0.093 mmol) was subject to chiral SFC (Column 1K, 4.6×100 mm, 5 mic, 50% EtOH/TFA co-solvent, 40° C.) to yield the title compounds as their respective bis-trifluoroacetic acid salt:

3-[[(5R)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[11.1.1]pentane-1-carbonitrile (7.54 mg, 0.0177 mmol) MS (m/z) 427.1 [M+H]+. ¹H NMR (400 MHz, DMSO-d6) δ 9.24 (s, 1H), 8.52 (s, 1H), 7.69-7.49 (m, 3H), 6.45 (d, J=16.7 Hz, 1H), 4.75 (q, J=6.6 Hz, 1H), 2.15-1.87 (m, 12H), 1.48 (d, J=6.5 Hz, 3H).

3-[[(5S)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[11.1.1]pentane-1-carbonitrile (9.96 mg, 0.0996 mmol). MS (m/z) 427.2 [M+H]+. ¹H NMR (400 MHz, DMSO-d6) δ 9.24 (s, 1H), 8.52 (s, 1H), 7.72-7.51 (m, 3H), 6.45 (d, J=16.6 Hz, 1H), 4.75 (q, J=6.6 Hz, 1H), 2.14-1.88 (m, 12H), 1.48 (d, J=6.6 Hz, 3H).

The following compounds were prepared using the procedure described for the synthesis of Compound 136 (E)-3-((4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile, with the appropriate modifications.

### Example 139

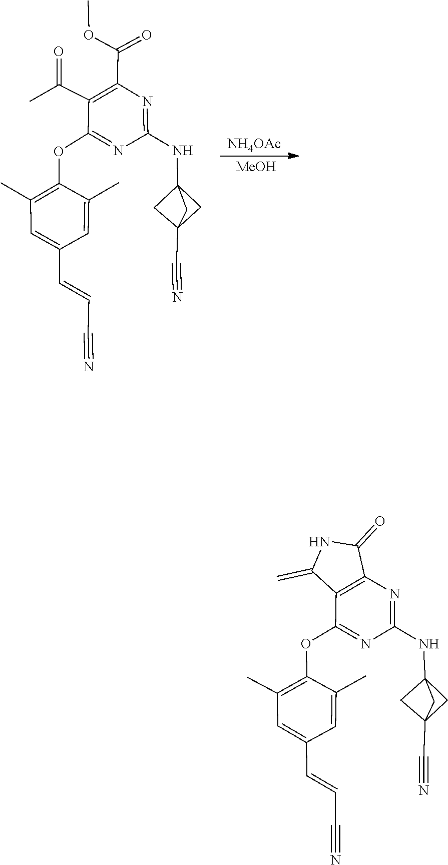 [SMILES: "MeOH" ; COC(=O)c1nc(NC23CC(C#N)(C2)C3)nc(Oc2c(C)cc(/C=C/C#N)cc2C)c1C(C)=O ; C=C1NC(=O)c2nc(NC34CC(C#N)(C3)C4)nc(Oc3c(C)cc(/C=C/C#N)cc3C)c21 ; "NH4OAc"]

### (E)-3-((4-(4-(2-cyanovinyl)-2,6-dimethylphenoxy)-5-methylene-7-oxo-6,7-dihydro-5H-pyrrolo[3,4-d]pyrimidin-2-yl)amino)bicyclo[1.1.1]pentane-1-carbonitrile—Compound 139

The reaction was carried out in the absence of sodium cyanoborohydride at 130° C. for 4 hours to afford the title compound (0.36 mg, 3.9% yield). MS (m/z) 425.2 [M+H]+. ¹H NMR (400 MHz, Acetonitrile-d₃) δ 8.80 (s, 1H), 7.52 (d, J=16.7 Hz, 1H), 7.44 (s, 2H), 6.84 (s, 1H), 6.10 (d, J=16.7 Hz, 1H), 5.34 (d, J=1.2 Hz, 1H), 5.05 (d, J=1.3 Hz, 1H), 2.16-2.01 (m, 12H).

### Example 140

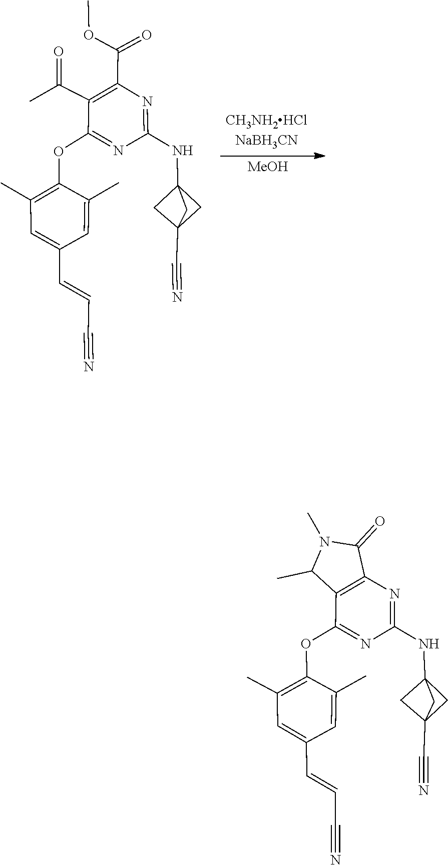 [SMILES: "MeOH" ; COC(=O)c1nc(NC23CC(C#N)(C2)C3)nc(Oc2c(C)cc(/C=C/C#N)cc2C)c1C(C)=O ; Cc1cc(/C=C/C#N)cc(C)c1Oc1nc(NC23CC(C#N)(C2)C3)nc2c1C(C)N(C)C2=O]

### (E)-3-((4-(4-(2-cyanovinyl)-2,6-dimethylphenoxy)-5,6-dimethyl-7-oxo-6,7-dihydro-5H-pyrrolo[3,4-d]pyrimidin-2-yl)amino)bicyclo[1.1.1]pentane-1-carbonitrile—Compound 140

Methylamine hydrochloride was used in place of ammonium acetate to afford the title compound as a trifluoroacetic acid salt (0.76 mg, 7.9% yield). MS (m/z) 441.3 [M+H]+. ¹H NMR (400 MHz, Acetonitrile-d3) δ 7.51 (d, J=16.7 Hz, 1H), 7.43 (s, 2H), 6.75 (s, 1H), 6.10 (d, J=16.7 Hz, 1H), 4.63 (q, J=6.6 Hz, 1H), 3.10 (s, 3H), 2.16-2.06 (m, 12H), 1.61 (d, J=6.6 Hz, 3H).

### Example 141

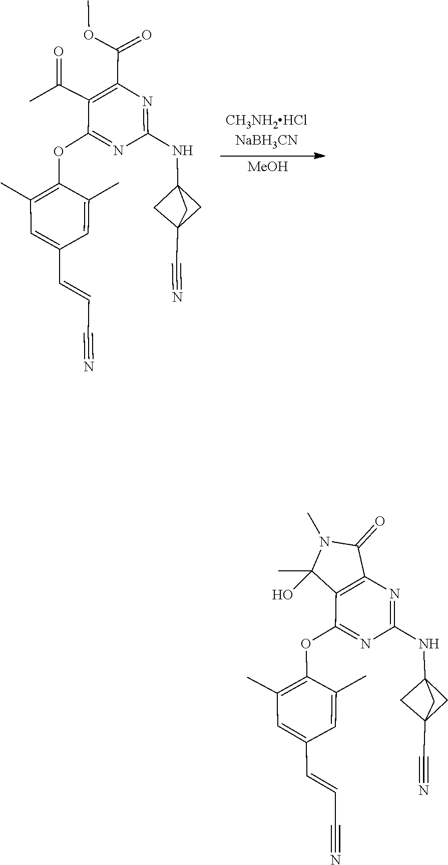 [SMILES: "MeOH" ; COC(=O)c1nc(NC23CC(C#N)(C2)C3)nc(Oc2c(C)cc(/C=C/C#N)cc2C)c1C(C)=O ; Cc1cc(/C=C/C#N)cc(C)c1Oc1nc(NC23CC(C#N)(C2)C3)nc2c1C(C)(O)N(C)C2=O]

### (E)-3-((4-(4-(2-cyanovinyl)-2,6-dimethylphenoxy)-5-hydroxy-5,6-dimethyl-7-oxo-6,7-dihydro-5H-pyrrolo[3,4-d]pyrimidin-2-yl)amino)bicyclo[1.1.1]pentane-1-carbonitrile—Compound 141

Methylamine hydrochloride was used in place of ammonium acetate. The residue was purified by preparative HPLC (0-100% MeCN in water) then lyophilized to afford the title compound (0.50 mg, 5.0% yield) MS (m/z) 457.0 [M+H]+. ¹H NMR (400 MHz, Acetonitrile-d3) δ 7.56-7.42 (m, 3H), 6.70 (s, 1H), 6.10 (d, J=16.7 Hz, 1H), 4.13 (s, 1H), 3.00 (s, 3H), 2.15-2.03 (m, 12H), 1.82 (s, 3H).

### Example 142

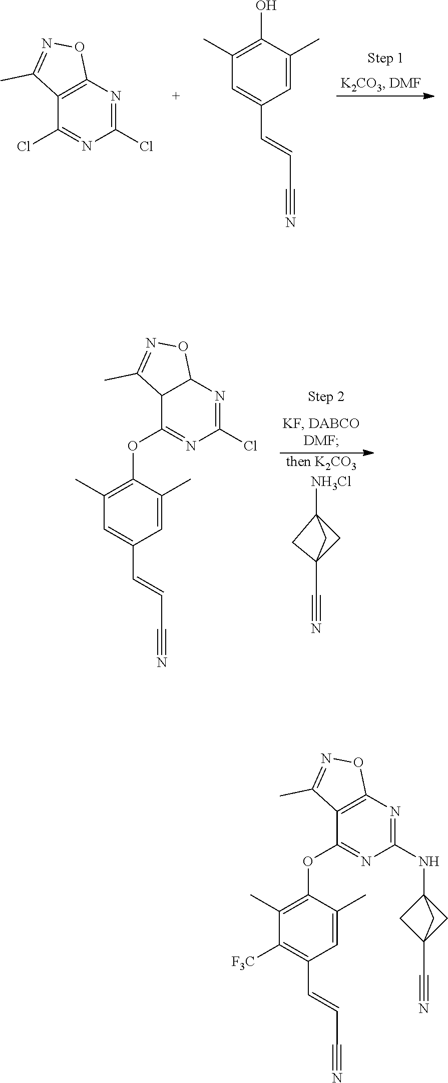

### (E)-3-[4-(6-chloro-3-methyl-isoxazolo[5,4-d]pyrimidin-4-yl)oxy-3,5-dimethyl-phenyl]prop-2-enenitrile (Intermediate 142a)

(E)-3-(4-hydroxy-3,5-dimethyl-phenyl)prop-2-enenitrile (1.47 mmol, 255 mg), 4,6-dichloro-3-methyl-isoxazolo[5,4-d]pyrimidine (1.47 mmol, 300 mg), and potassium carbonate (1.62 mmol, 224 mg) were suspended in DMF (10 mL). The mixture was stirred at room temperature for two hours. The mixture was diluted with water and EtOAc. The aqueous phase was extracted twice with EtOAc. The organic phase was washed with brine, dried over MgSO₄, filtered, and concentrated in vacuo. The resulting residue was purified by silica gel chromatography (0-20% MeOH/DCM) to afford the title compound. 1H NMR (400 MHz, Acetonitrile-d3) δ 7.51 (d, J=16.7 Hz, 1H), 7.43 (s, 2H), 6.12 (d, J=16.7 Hz, 1H), 2.73 (s, 3H), 2.18 (s, 6H).

### Step 2, preparation of (E)-3-((4-(4-(2-cyanovinyl)-2,6-dimethylphenoxy)-3-methylisoxazolo[5,4-d]pyrimidin-6-yl)amino)bicyclo[1.1.1]pentane-1-carbonitrile—Compound 142

Intermediate 142a (0.0883 mmol, 30.1 mg), potassium fluoride (0.441 mmol, 25.6 mg), and 1,4-diazabicyclo[2.2.2]octane (0.0441, mmol, 4.95 mg) were suspended in DMF (0.5 mL). The mixture was stirred at 50° C. for two hours and then cooled to room temperature. To the mixture was added 3-aminobicyclo[1.1.1]pentane-1-carbonitrile; hydrochloride (0.0881 mmol, 28.6 mg) and potassium carbonate (0.441 mmol, 60.9 mg). The mixture was stirred at room temperature for one hour. At which point, the mixture was filtered and purified by preparative HPLC (0-100% MeCN in water, 0.1% TFA) then lyophilized to afford the title compound (2 mg, 5.5% yield). ¹H NMR (400 MHz, Acetonitrile-d₃) δ 7.51 (d, J=16.7 Hz, 1H), 7.43 (s, 2H), 6.80 (s, 1H), 6.10 (d, J=16.6 Hz, 1H), 2.60 (s, 3H), 2.19-2.01 (m, 12H). [Compound does not ionize]

### Example 143

The following compounds were prepared using the procedure described for the synthesis of 3-[[4-[4-[(E)-2-cyanovinyl]-2,6-dimethyl-phenoxy]-2-(2-methoxyethyl)pyrazolo[3,4-d]pyrimidin-6-yl]amino]bicyclo[1.1.1]pentane-1-carbonitrile, Example 123, with the appropriate modifications.

### Ethyl 3-amino-1-ethylpyrazole-4-carboxylate

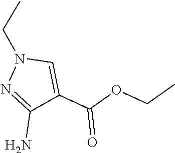

Ethylhydrazine oxalate was used in place of (2-methoxyethyl)hydrazine. MS (m/z) 184.10 [M+H]+.

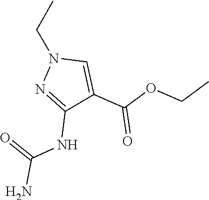 [SMILES: CCOC(=O)c1cn(CC)nc1NC(N)=O]

Ethyl 3-(carbamoylamino)-1-ethylpyrazole-4-carboxylate was used in place of Intermediate 123a. MS (m/z) 227.20 [M+H]+.

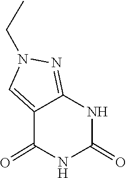

2-ethyl-5H,7H-pyrazolo[3,4-d]pyrimidine-4,6-dione was used in place of Intermediate 123b. MS (m/z) 181.10 [M+H]+.

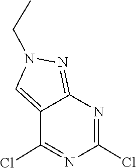

4,6-dichloro-2-ethylpyrazolo[3,4-d]pyrimidine was used in place of Intermediate 123c. MS (m/z) 217.05, 219.05 [M+H]⁺. ¹H NMR (400 MHz, Chloroform-d) δ 8.23 (s, 1H), 4.55 (q, J=7.3 Hz, 2H), 1.69 (t, J=7.3 Hz, 3H).

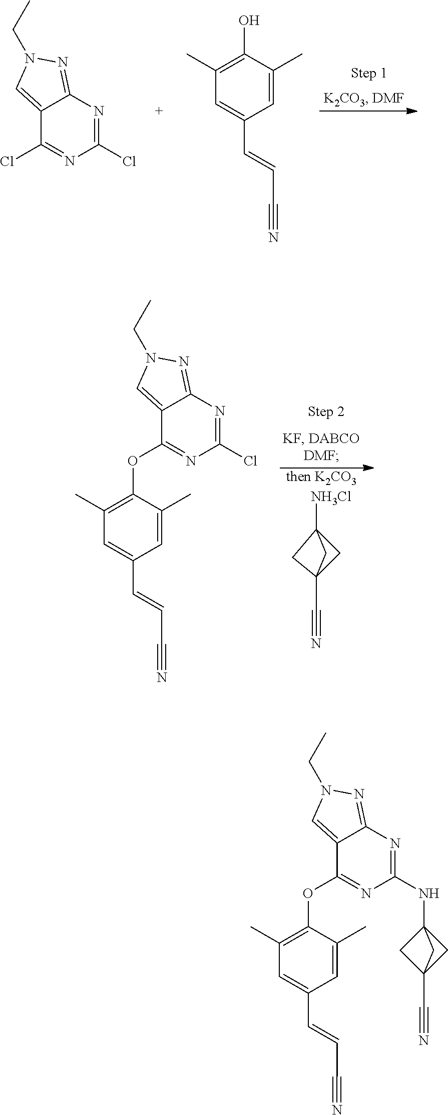

### Step 1, preparation of (E)-3-[4-(6-chloro-2-ethyl-pyrazolo[3,4-d]pyrimidin-4-yl)oxy-3,5-dimethyl-phenyl]prop-2-enenitrile (Intermediate 143a)

(E)-3-(4-hydroxy-3,5-dimethyl-phenyl)prop-2-enenitrile (0.921 mmol, 160 mg), 4,6-dichloro-2-ethyl-pyrazolo[3,4-d]pyrimidine (0.921 mmol, 200 mg), and potassium carbonate (0.921 mmol, 127 mg) were suspended in DMF (5 mL). The mixture was stirred at room temperature overnight. At which point, the mixture was diluted with water and EtOAc. The aqueous phase was extracted twice with EtOAc. The organic phase was washed with brine, dried over MgSO4, filtered, and concentrated in vacuo to yield the title compound. MS (m/z) 354.2 [M+H]+. ¹H NMR (400 MHz, DMSO-d6) δ 8.91 (s, 1H), 7.63 (d, J=16.7 Hz, 1H), 7.53 (s, 2H), 6.46 (d, J=16.7 Hz, 1H), 4.50 (q, J=7.3 Hz, 2H), 2.10 (s, 6H), 1.54 (t, J=7.2 Hz, 3H).

### Step 2, Preparation of 3-[[4-[4-[(E)-2-cyanovinyl]-2,6-dimethyl-phenoxy]-2-ethyl-pyrazolo[3,4-d]pyrimidin-6-yl]amino]bicyclo[1.1.1]pentane-1-carbonitrile—Compound 143

Intermediate 143a (0.111 mmol, 39.4 mg), potassium fluoride (0.557 mmol, 32.3 mg), and 1,4-diazabicyclo[2.2.2]octane (0.0557 mmol, 6.25 mg) were suspended in NMP (1 mL). The mixture was stirred at 60° C. overnight. To the mixture was added 3-aminobicyclo[1.1.1]pentane-1-carbonitrile; hydrochloride (0.334 mmol, 48.3 mg) and potassium carbonate (0.557 mmol, 77.0 mg). The mixture stirred at room temperature for four hours. The mixture was filtered and purified by preparative HPLC (0-100% MeCN in water, 0.1% TFA) then lyophilized to afford the title compound (2.60 mg, 5.5% yield). MS (m/z) 426.3 [M+H]+. ¹H NMR (400 MHz, Acetonitrile-d3) δ 8.08 (s, 1H), 7.51 (d, J=16.7 Hz, 1H), 7.41 (s, 2H), 6.14-6.05 (m, 2H), 4.34 (q, J=7.3 Hz, 2H), 2.27 (s, 6H), 2.14 (s, 6H), 1.55 (t, J=7.3 Hz, 3H).

The following compounds were prepared using the two-step procedure described for the synthesis of 3-[[4-[4-[(E)-2-cyanovinyl]-2,6-dimethyl-phenoxy]-2-ethyl-pyrazolo[3,4-d]pyrimidin-6-yl]amino]bicyclo[1.1.1]pentane-1-carbonitrile, with the appropriate modifications.

### Example 144

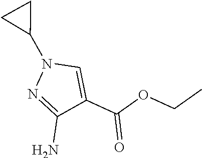

### Ethyl 3-amino-1-cyclopropylpyrazole-4-carboxylate

Cyclopropylhydrazine hydrochloride was used in place of (2-methoxyethyl)hydrazine. MS (m/z) 196.20 [M+H]+.

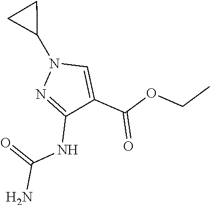 [SMILES: CCOC(=O)c1cn(C2CC2)nc1NC(N)=O]

Ethyl 3-(carbamoylamino)-1-cyclopropylpyrazole-4-carboxylate was used in place of Intermediate 123a. MS (m/z) 239.10 [M+H]+.

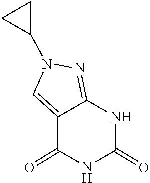 [SMILES: O=c1[nH]c(=O)c2cn(C3CC3)nc2[nH]1]

2-cyclopropyl-5H,7H-pyrazolo[3,4-d]pyrimidine-4,6-dione was used in place of Intermediate 123b. MS (m/z) 193.15 [M+H]+.

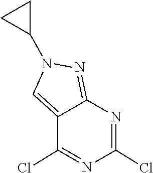 [SMILES: Clc1nc(Cl)c2cn(C3CC3)nc2n1]

4,6-dichloro-2-cyclopropylpyrazolo[3,4-d]pyrimidine was used in place of Intermediate 123c. MS (m/z) 228.90, 230.90 [M+H]⁺. ¹H NMR (400 MHz, Chloroform-d) δ 8.28 (s, 1H), 4.04-3.98 (m, 1H), 1.57-1.44 (m, 2H), 1.37-1.21 (m, 2H).

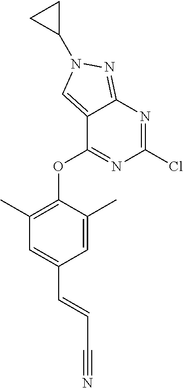 [SMILES: Cc1cc(/C=C/C#N)cc(C)c1Oc1nc(Cl)nc2nn(C3CC3)cc12]

### (E)-3-[4-(6-chloro-2-cyclopropyl-pyrazolo[3,4-d]pyrimidin-4-yl)oxy-3,5-dimethyl-phenyl]prop-2-enenitrile (Intermediate 144a)

4,6-dichloro-2-cyclopropyl-pyrazolo[3,4-d]pyrimidine was used in place of 4,6-dichloro-2-ethyl-pyrazolo[3,4-d]pyrimidine. Residue was purified by silica gel chromatography (0-100% EtOAc/Hexane) to afford the title compound (191 mg, 60% yield). MS (m/z) 366.2 [M+H]+. ¹H NMR (400 MHz, DMSO-d6) δ 9.01 (s, 1H), 7.63 (d, 1H), 7.53 (s, 2H), 6.46 (d, J=16.7 Hz, 1H), 4.23 (tt, J=7.6, 3.9 Hz, 1H), 2.10 (s, 6H), 1.37 (dt, J=4.8, 4.4 Hz, 2H), 1.19 (dt, J=7.4, 3.7 Hz, 2H).

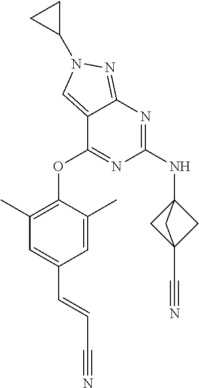 [SMILES: Cc1cc(/C=C/C#N)cc(C)c1Oc1nc(NC23CC(C#N)(C2)C3)nc2nn(C3CC3)cc12]

### (E)-3-((4-(4-(2-cyanovinyl)-2,6-dimethylphenoxy)-2-cyclopropyl-2H-pyrazolo[3,4-d]pyrimidin-6-yl)amino)bicyclo[1.1.1]pentane-1-carbonitrile—Compound 144

Intermediate 144a was used in place of Intermediate 143a to afford the title compound (1.06 mg, 2.7% yield). MS (m/z) 438.3 [M+H]+. ¹H NMR (400 MHz, Acetonitrile-d3) δ 8.16 (s, 1H), 7.50 (d, J=16.7 Hz, 1H), 7.41 (s, 2H), 6.15-6.05 (m, 2H), 3.91 (tt, J=7.5, 3.8 Hz, 1H), 2.26 (s, 6H), 2.14 (s, 6H), 1.32-1.27 (m, 2H), 1.15-1.08 (m, 2H).

### Example 145

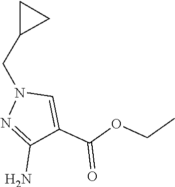

### Ethyl 3-amino-1-(cyclopropylmethyl)-1H-pyrazole-4-carboxylate

(Cyclopropylmethyl)hydrazine dihydrochloride was used in place of (2-methoxyethyl)hydrazine. MS (m/z) 210.10 [M+H]+.

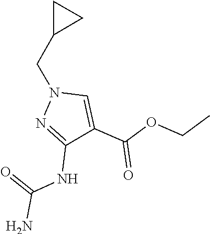 [SMILES: CCOC(=O)c1cn(CC2CC2)nc1NC(N)=O]

Ethyl 1-(cyclopropylmethyl)-3-ureido-1H-pyrazole-4-carboxylate was used in place of Intermediate 123a. MS (m/z) 253.25 [M+H]+.

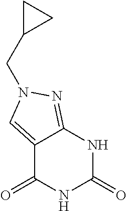 [SMILES: O=c1[nH]c(=O)c2cn(CC3CC3)nc2[nH]1]

2-(cyclopropylmethyl)-2,7-dihydro-4H-pyrazolo[3,4-d]pyrimidine-4,6(5H)-dione was used in place of Intermediate 123b. MS (m/z) 207.20 [M+H]+.

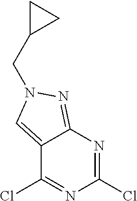 [SMILES: Clc1nc(Cl)c2cn(CC3CC3)nc2n1]

4,6-dichloro-2-(cyclopropylmethyl)-2H-pyrazolo[3,4-d]pyrimidine was used in place of Intermediate 123c. MS (m/z) 243.05, 245.05 [M+H]⁺. ¹H NMR (400 MHz, Chloroform-d) δ 8.31 (s, 1H), 4.34 (d, J=7.4 Hz, 2H), 1.49 (pt, J=7.6, 4.8 Hz, 1H), 0.88-0.76 (m, 2H), 0.55 (dt, J=6.2, 4.9 Hz, 2H).

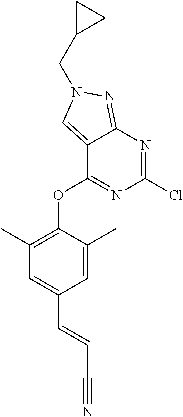 [SMILES: Cc1cc(/C=C/C#N)cc(C)c1Oc1nc(Cl)nc2nn(CC3CC3)cc12]

### (E)-3-[4-[6-chloro-2-(cyclopropylmethyl)pyrazolo[3,4-d]pyrimidin-4-yl]oxy-3,5-dimethyl-phenyl]prop-2-enenitrile (Intermediate 145a)

4,6-dichloro-2-(cyclopropylmethyl)pyrazolo[3,4-d]pyrimidine was used in place of 4,6-dichloro-2-ethyl-pyrazolo[3,4-d]pyrimidine. Residue was purified by silica gel chromatography (0-100% EtOAc/Hexane) to afford the title compound (217 mg, 69% yield). MS (m/z) 380.2 [M+H]+. ¹H NMR (400 MHz, DMSO-d6) δ 8.90 (s, 1H), 7.64 (d, J=16.7 Hz, 1H), 7.54 (s, 2H), 6.47 (d, J=16.7 Hz, 1H), 4.33 (d, J=7.3 Hz, 2H), 2.11 (s, 6H), 1.43 (tt, J=7.6, 4.7 Hz, 1H), 0.61 (dt, J=7.9, 3.0 Hz, 2H), 0.50 (dt, 2H).

### (E)-3-((4-(4-(2-cyanovinyl)-2,6-dimethylphenoxy)-2-(cyclopropylmethyl)-2H-pyrazolo[3,4-d]pyrimidin-6-yl)amino)bicyclo[1.1.1]pentane-1-carbonitrile—Compound 145

Intermediate 145a was used in place of Intermediate 143a to afford the title compound (2.6 mg, 7.1% yield). MS (m/z) 452.3 [M+H]+. ¹H NMR (400 MHz, DMSO-d6) δ 8.55 (s, 1H), 7.81 (s, 1H), 7.63 (d, J=16.7 Hz, 1H), 7.54 (s, 2H), 6.44 (d, J=16.7 Hz, 1H), 4.14 (d, J=7.2 Hz, 2H), 2.41-1.95 (m, 12H), 1.38 (tq, J=12.2, 7.4, 6.1 Hz, 1H), 0.59 (dt, J=8.1, 3.0 Hz, 2H), 0.46 (dt, 2H).

### Example 146

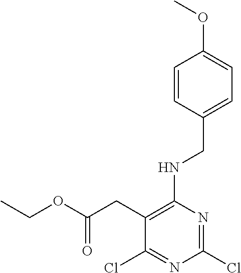 [SMILES: CCOC(=O)Cc1c(Cl)nc(Cl)nc1NCc1ccc(OC)cc1]

(4-Methoxyphenyl)methanamine (7.5 mL, 57.14 mmol) and N, N′-diisopropylethylamine (10.9 mL, 62.33 mmol) was added to a solution of ethyl 2-(2,4,6-trichloropyrimidin-5-yl)acetate (14.0 g, 51.95 mmol) in DMF (60 mL). The mixture was stirred at 60° C. for 1 h, quenched by water and then extracted with EtOAc. The organic phases were combined, washed with 1N HCl and then brine, dried over anhydrous Na₂SO₄, evaporated and purified by silica gel flash column chromatography to afford intermediate 146-1 (MS (m/z) 369.8 [M+H]+. ¹H NMR (400 MHz, CDCl3) δ 7.29 (d, J=8.5 Hz, 2H), 6.91 (d, J=8.6 Hz, 2H), 6.15 (s, 1H), 4.63 (d, J=5.2 Hz, 2H), 4.16 (q, J=7.1 Hz, 2H), 3.83 (s, 3H), 3.62 (s, 2H), 1.23 (t, J=7.2 Hz, 3H).

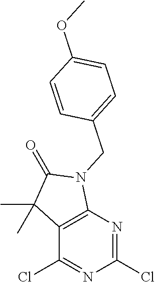 [SMILES: COc1ccc(CN2C(=O)C(C)(C)c3c(Cl)nc(Cl)nc32)cc1]

Intermediate 146-1 (10.0 g, 27.0 mmol) and DMF (150 mL) were added to a round bottom flask. Cesium carbonate (8.15 g, 25.0 mmol) was added to the solution under N₂ at room temperature, then heated to 40° C. One hour later, methyl iodide (1.42 g, 10 mmol) was added to the mixture, and stirred for another 1 h at 40° C. Then the reaction was quenched by water and extracted with EtOAc. The organic phases were combined, washed by brine, dried over Na₂SO₄, filtered, concentrated in vacuo, purified by silica gel flash column chromatography, and triturated with petroleum ether to afford 146b (8.7 g, 91% yield). MS (m/z) 352.2 [M+H]+. ¹H NMR (400 MHz, Chloroform-d) δ 7.42-7.33 (m, 2H), 6.89-6.80 (m, 2H), 4.86 (s, 2H), 3.78 (s, 3H), 1.48 (s, 6H).

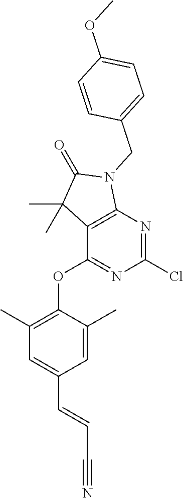 [SMILES: COc1ccc(CN2C(=O)C(C)(C)c3c(Oc4c(C)cc(/C=C/C#N)cc4C)nc(Cl)nc32)cc1]

### (E)-3-[4-[2-chloro-7-[(4-methoxyphenyl)methyl]-5,5-dimethyl-6-oxo-pyrrolo[2,3-d]pyrimidin-4-yl]oxy-3,5-dimethyl-phenyl]prop-2-enenitrile (Intermediate 146a)

2,4-dichloro-7-[(4-methoxyphenyl)methyl]-5,5-dimethyl-pyrrolo[2,3-d]pyrimidin-6-one was used in place of 4,6-dichloro-2-ethyl-pyrazolo[3,4-d]pyrimidine. Residue was purified by silica gel chromatography (0-100% EtOAc/Hexane) to afford the title compound. MS (m/z) 489.1 [M+H]+. ¹H NMR (400 MHz, Acetonitrile-d3) δ 7.52 (d, J=16.7 Hz, 1H), 7.41 (s, 2H), 7.10 (d, 2H), 6.83 (d, 2H), 6.11 (d, J=16.7 Hz, 1H), 4.67 (s, 2H), 3.79 (s, 3H), 2.13 (s, 6H), 1.45 (s, 6H).

### (E)-3-((4-(4-(2-cyanovinyl)-2,6-dimethylphenoxy)-7-(4-methoxybenzyl)-5,5-dimethyl-6-oxo-6,7-dihydro-5H-pyrrolo[2,3-d]pyrimidin-2-yl)amino)bicyclo[1.1.1]pentane-1-carbonitrile—Compound 146

Intermediate 146a was used in place of Intermediate 143a to afford the title compound (2.6 mg, 6.5% yield). MS (m/z) 561.2 [M+H]+. ¹H NMR (400 MHz, Acetonitrile-d3) δ 7.50 (d, J=16.7 Hz, 1H), 7.40 (s, 2H), 7.25 (d, 2H), 6.89 (d, 2H), 6.07 (d, J=16.7 Hz, 1H), 5.92 (s, 1H), 4.77 (s, 2H), 3.79 (s, 3H), 2.14-2.10 (m, 12H), 1.32 (s, 6H).

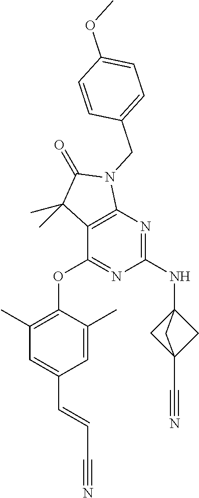 [SMILES: COc1ccc(CN2C(=O)C(C)(C)c3c(Oc4c(C)cc(/C=C/C#N)cc4C)nc(NC45CC(C#N)(C4)C5)nc32)cc1]

### Example 147

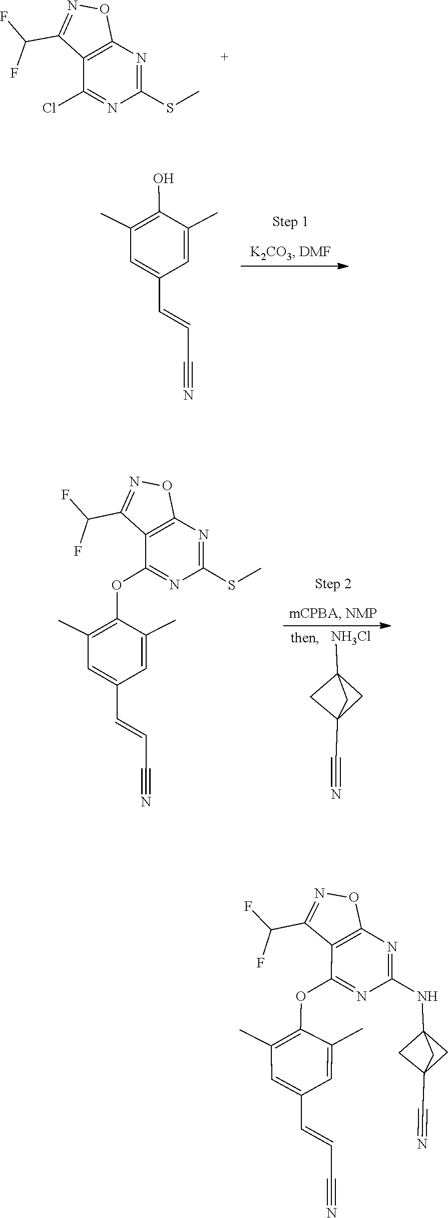

### Step 1, preparation of (E)-3-[4-[3-(difluoromethyl)-6-methylsulfanyl-isoxazolo[5,4-d]pyrimidin-4-yl]oxy-3,5-dimethyl-phenyl]prop-2-enenitrile (Intermediate 147a)

(E)-3-(4-hydroxy-3,5-dimethyl-phenyl)prop-2-enenitrile (1.19 mmol, 206 mg), 4-chloro-3-(difluoromethyl)-6-methylsulfanyl-isoxazolo[5,4-d]pyrimidine (1.19 mmol, 300 mg), and potassium carbonate (1.31 mmol, 181 mg) were suspended in DMF (7 mL). The mixture was stirred at room temperature overnight. At which point, the mixture was diluted with water and EtOAc. The aqueous phase was extracted twice with EtOAc. The organic phase was washed with brine, dried over MgSO₄, filtered, and concentrated in vacuo to yield the title compound (451 mg, 97.3% yield) MS (m/z) 389.1 [M+H]+. ¹H NMR (400 MHz, Acetonitrile-d₃) δ 7.51 (d, 1H), 7.41 (s, 2H), 7.21 (t, 1H), 6.11 (d, J=16.8, 0.8 Hz, 1H), 2.46 (s, 3H), 2.15 (s, 6H).

### Step 2, Preparation of 3-[[4-[4-[(E)-2-cyanovinyl]-2,6-dimethyl-phenoxy]-3-(difluoromethyl)isoxazolo[5,4-d]pyrimidin-6-yl]amino]bicyclo[1.1.1]pentane-1-carbonitrile—Compound 147

To Intermediate 147a (0.0631 mmol, 24.5 mg) in NMP (0.6 mL) was added 3-chloroperoxybenzoic acid (0.158 mmol, 27.2 mg). The reaction mixture was stirred at room temperature overnight. Potassium carbonate (0.315 mmol, 43.6 mg) and 3-aminobicyclo[1.1.1]pentane-1-carbonitrile; hydrochloride (0.0881 mmol, 28.6 mg) was added to the reaction mixture and stirred at 60° C. for 1.5 hours. The mixture was quenched with water, filtered, and purified by preparative HPLC (0-100% MeCN in water) then lyophilized to afford the title compound (0.32 mg, 1.13% yield). MS (m/z) 449.0 [M+H]+. ¹H NMR (400 MHz, Acetonitrile-d3) δ 7.52 (d, J=16.7 Hz, 1H), 7.45 (s, 2H), 7.12 (t, J=52.6 Hz, 1H), 7.04 (s, 1H), 6.11 (d, J=16.7 Hz, 1H), 2.15 (s, 6H), 2.08 (s, 6H).

### Example 148

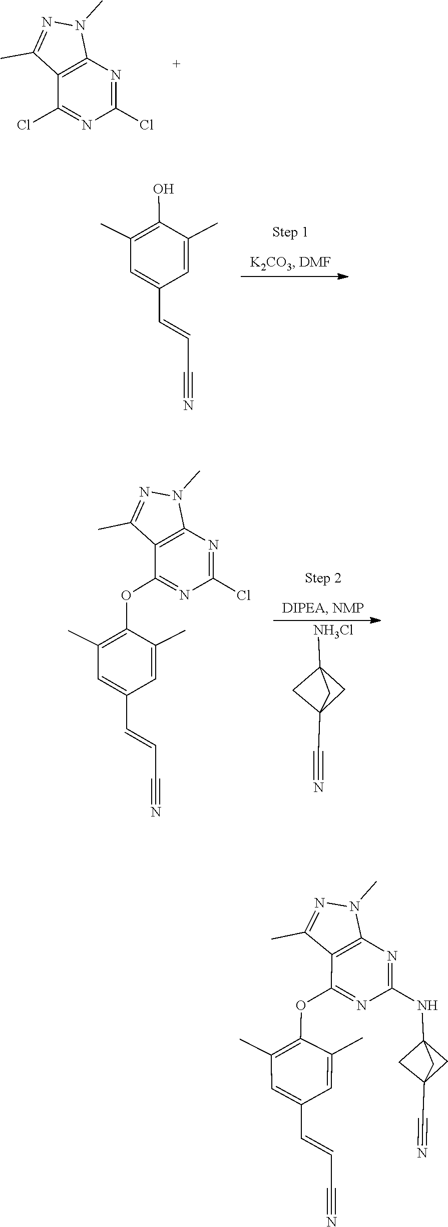

### Step 1, preparation of (E)-3-[4-(6-chloro-1,3-dimethyl-pyrazolo[3,4-d]pyrimidin-4-yl)oxy-3,5-dimethyl-phenyl]prop-2-enenitrile (Intermediate 148a)

(E)-3-(4-hydroxy-3,5-dimethyl-phenyl)prop-2-enenitrile (1.38 mmol, 239 mg), 4,6-dichloro-1,3-dimethyl-pyrazolo[3,4-d]pyrimidine (1.38 mmol, 300 mg), and potassium carbonate (1.66 mmol, 229 mg) were suspended in DMF (7 mL). The mixture was stirred at room temperature overnight. The mixture was diluted with water (10 mL) and EtOAc (20 mL). The aqueous phase was extracted twice with EtOAc. The organic phase was washed with water and brine, dried over MgSO₄, filtered, and concentrated in vacuo. The resulting residue was purified by silica gel chromatography (0-100% EtOAc/Hexane) to afford the title compound (438 mg, 94% yield). MS (m/z) 354.1 [M+H]+. ¹H NMR (400 MHz, Acetonitrile-d3) δ 7.50 (d, J=16.7 Hz, 1H), 7.41 (s, 2H), 6.10 (d, J=16.7 Hz, 1H), 3.95 (s, 3H), 2.69 (s, 3H), 2.16 (s, 6H).

### Step 2, preparation of (E)-3-((4-(4-(2-cyanovinyl)-2,6-dimethylphenoxy)-1,3-dimethyl-1H-pyrazolo[3,4-d]pyrimidin-6-yl)amino)bicyclo[1.1.1]pentane-1-carbonitrile—Compound 148

Intermediate 148a (0.151 mmol, 53.3 mg), 3-aminobicyclo[1.1.1]pentane-1-carbonitrile; hydrochloride (0.0881 mmol, 28.6 mg), N,N-diisopropylethylamine (0.904 mmol, 0.157 mL) were suspended in NMP (1.5 mL). The mixture was stirred at 140° C. overnight. The mixture was filtered and purified by preparative HPLC (0-100% MeCN in water) then lyophilized to afford the title compound (8.9 mg, 13.9% yield). MS (m/z) 426.2 [M+H]+. ¹H NMR (400 MHz, Acetonitrile-d3) δ 7.51 (d, J=16.7 Hz, 1H), 7.41 (s, 2H), 6.23 (s, 1H), 6.09 (d, J=16.7 Hz, 1H), 3.77 (s, 3H), 2.58 (s, 3H), 2.15 (d, J=2.5 Hz, 12H).

### Example 149

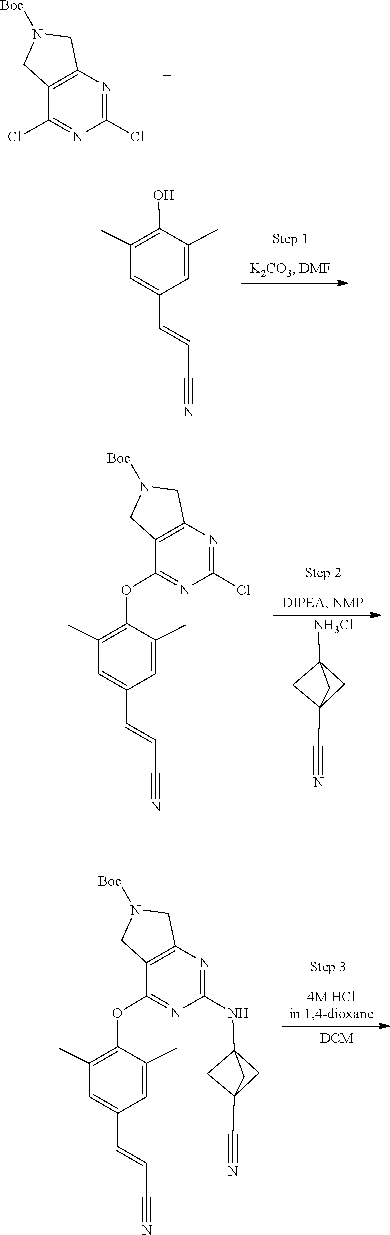

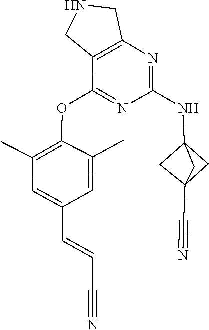 [SMILES: Cc1cc(/C=C/C#N)cc(C)c1Oc1nc(NC23CC(C#N)(C2)C3)nc2c1CNC2]

### Step 1, preparation of tert-butyl 2-chloro-4-[4-[(E)-2-cyanovinyl]-2,6-dimethyl-phenoxy]-5,7-dihydropyrrolo[3,4-d]pyrimidine-6-carboxylate (Intermediate 149a)

(E)-3-(4-hydroxy-3,5-dimethyl-phenyl)prop-2-enenitrile (5.17 mmol, 895 mg), tert-butyl 2,4-dichloro-5,7-dihydropyrrolo[3,4-d]pyrimidine-6-carboxylate (5.17 mmol, 1.5 g), and potassium carbonate (601 mmol, 857 mg) were suspended in DMF (25 mL). The mixture was stirred at room temperature overnight. The mixture was diluted with water and EtOAc. The aqueous phase was extracted twice with EtOAc. The organic phase was washed with water and brine, dried over MgSO₄, filtered, and concentrated in vacuo. The resulting residue was purified by silica gel chromatography (0-100% EtOAc/Hexane) to afford the title compound (601 mg, 27% yield). MS (m/z) 426.9 [M+H]+. ¹H NMR (400 MHz, Acetonitrile-d₃) δ 7.49 (d, J=16.7 Hz, 1H), 7.39 (s, 2H), 6.10 (d, J=16.7 Hz, 1H), 4.65 (dd, 4H), 2.14 (s, 6H), 1.52 (s, 9H).

### Step 2, preparation of tert-butyl 2-[(3-cyano-1-bicyclo[1.1.1]pentanyl)amino]-4-[4-[(E)-2-cyanovinyl]-2,6-dimethyl-phenoxy]-5,7-dihydropyrrolo[3,4-d]pyrimidine-6-carboxylate (Intermediate 149b)

Intermediate 149a (1.20 mmol, 510 mg), 3-aminobicyclo[1.1.1]pentane-1-carbonitrile; hydrochloride (3.59 mmol, 388 mg), and N,N-diisopropylethylamine (7.17 mmol, 1.25 mL) were suspended in NMP (10 mL). The mixture was stirred at 140° C. overnight. The mixture was diluted with water and EtOAc. The aqueous phase was extracted twice with EtOAc. The organic phase was washed with water and brine, dried over MgSO₄, filtered, and concentrated in vacuo. The resulting residue was purified by silica gel chromatography (0-100% EtOAc/Hexane) to afford the title compound (200 mg, 41% yield). MS (m/z) 499.0 [M+H]+. ¹H NMR (400 MHz, Acetonitrile-d3) δ 7.50 (d, J=16.7 Hz, 1H), 7.40 (s, 2H), 6.34 (s, 1H), 6.08 (d, J=16.7 Hz, 1H), 4.59 (d, J=10.8 Hz, 2H), 4.42 (d, J=7.7 Hz, 2H), 2.14-2.03 (m, 12H), 1.51 (s, 9H).

### Step 3, Preparation of 3-[[4-[4-[(E)-2-cyanovinyl]-2,6-dimethyl-phenoxy]-6,7-dihydro-5H-pyrrolo[3,4-d]pyrimidin-2-yl]amino]bicyclo[1.1.1]pentane-1-carbonitrile—Compound 149

To Intermediate 149b (0.401 mmol, 200 mg) in DCM (3 mL) was added 4M HCl in 1,4-dioxane (1 mL). The mixture was stirred at room temperature for 1 hour. The mixture was then concentrated in vacuo, redissolved in ACN/water, filtered, and purified via preparative HPLC (0-100% MeCN in water, 0.1% TFA) then lyophilized to afford the title compound as the bis-trifluoroacetic acid salt (35 mg, 22% yield). MS (m/z) 399.2 [M+H]+. ¹H NMR (400 MHz, Acetonitrile-d₃) δ 9.05 (s, 1H), 7.50 (d, J=16.7 Hz, 1H), 7.41 (s, 2H), 6.57 (s, 1H), 6.09 (d, J=16.7 Hz, 1H), 4.64 (s, 2H), 4.40 (s, 2H), 2.13 (s, 6H), 2.09 (s, 6H).

### Example 150

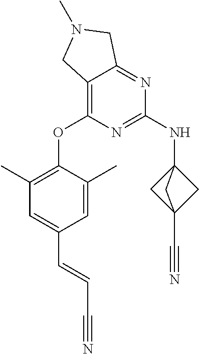 [SMILES: Cc1cc(/C=C/C#N)cc(C)c1Oc1nc(NC23CC(C#N)(C2)C3)nc2c1CN(C)C2]

### (E)-3-((4-(4-(2-cyanovinyl)-2,6-dimethylphenoxy)-6-methyl-6,7-dihydro-5H-pyrrolo[3,4-d]pyrimidin-2-yl)amino)bicyclo[1.1.1]pentane-1-carbonitrile—Compound 150

Compound 149 (0.0376 mmol, 15 mg), 37% wt formaldehyde in water (0.113 mmol, 0.0843 mL), acetic acid (1.75 mmol, 0.1 mL), and sodium triacetoxyborohydride (0.113 mmol, 23.9 mg) were suspended in MeOH/DCE (1:3) (0.4 mL). The mixture was stirred at room temperature for 10 minutes. The mixture was quenched with aq. Na₂CO₃, filtered, and purified via preparative HPLC (0-100% MeCN in water, 0.1% TFA) then lyophilized to afford the title compound as the mono-trifluoroacetic acid salt (6.47 mg, 41% yield). MS (m/z) 413.2 [M+H]+. ¹H NMR (400 MHz, DMSO-d6) δ 8.47 (s, 1H), 7.64 (d, J=16.6 Hz, 1H), 7.56 (s, 2H), 6.44 (d, J=16.7 Hz, 1H), 4.86 (s, 1H), 4.63 (s, 1H), 4.48 (s, 1H), 4.42 (s, 1H), 3.08 (s, 3H), 2.15-1.89 (m, 12H).

### Example 151

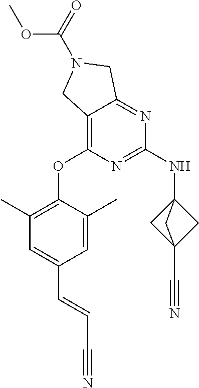 [SMILES: COC(=O)N1Cc2nc(NC34CC(C#N)(C3)C4)nc(Oc3c(C)cc(/C=C/C#N)cc3C)c2C1]

### (E)-methyl 2-((3-cyanobicyclo[1.1.1]pentan-1-yl)amino)-4-(4-(2-cyanovinyl)-2,6-dimethylphenoxy)-5H-pyrrolo[3,4-d]pyrimidine-6(7H)-carboxylate—Compound 151

Compound 149 (0.0376 mmol, 15 mg) and potassium carbonate (0.0615 mmol, 8.49 mg) were suspended in DMF (0.4 mL). Methyl chloroformate (0.0293 mmol, 2.27 μL) was added dropwise. The mixture was left to stir at room temperature for 20 minutes. The mixture was treated with 0.5 mL of water, filtered, and purified via preparative HPLC (0-100% MeCN in water, 0.1% TFA) then lyophilized to afford the title compound as the mono-trifluoroacetic acid salt (1.60 mg, 12% yield). MS (m/z) 457.2 [M+H]+. ¹H NMR (400 MHz, Acetonitrile-d3) δ 7.50 (d, J=16.7 Hz, 1H), 7.41 (s, 2H), 6.90 (s, 1H), 6.09 (d, J=16.7 Hz, 1H), 4.66 (s, 2H), 4.49 (s, 2H), 3.75 (s, 3H), 2.13 (s, 6H), 2.09 (s, 6H).

### Example 152

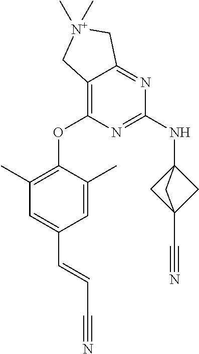 [SMILES: Cc1cc(/C=C/C#N)cc(C)c1Oc1nc(NC23CC(C#N)(C2)C3)nc2c1C[N+](C)(C)C2]

### (E)-2-((3-cyanobicyclo[1.1.1]pentan-1-yl)amino)-4-(4-(2-cyanovinyl)-2,6-dimethylphenoxy)-6,6-dimethyl-6,7-dihydro-5H-pyrrolo[3,4-d]pyrimidin-6-ium—Compound 152

Compound 149 (0.0585 mmol, 30 mg) and potassium carbonate (0.117 mmol, 16.2 mg) were suspended in DMF (1 mL). Iodomethane (0.117 mmol, 7.28 μL) was added dropwise. The mixture was left to stir at room temperature for 2 hours. Then, the mixture was treated with 0.5 mL of water, filtered, and purified via preparative HPLC (0-100% MeCN in water, 0.1% TFA) then lyophilized to afford the title compound as the mono-trifluoroacetic acid salt (16.8 mg, 67% yield). MS (m/z) 427.2 [M]+. ¹H NMR (400 MHz, Acetonitrile-d₃) δ 7.51 (d, J=16.7 Hz, 1H), 7.42 (s, 2H), 6.72 (s, 1H), 6.10 (d, J=16.7 Hz, 1H), 4.84 (s, 2H), 4.59 (s, 2H), 3.36 (s, 6H), 2.15 (s, 6H), 2.08 (s, 6H).

### Example 153

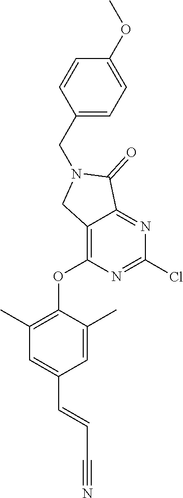 [SMILES: COc1ccc(CN2Cc3c(Oc4c(C)cc(/C=C/C#N)cc4C)nc(Cl)nc3C2=O)cc1]

### (E)-3-(4-((2-chloro-6-(4-methoxybenzyl)-7-oxo-6,7-dihydro-5H-pyrrolo[3,4-d]pyrimidin-4-yl)oxy)-3,5-dimethylphenyl)acrylonitrile—Intermediate 153a

2,4-dichloro-6-(4-methoxybenzyl)-5,6-dihydro-7H-pyrrolo[3,4-d]pyrimidin-7-one (250 mg, 0.771 mmol), (E)-3-(4-hydroxy-3,5-dimethyl-phenyl)prop-2-enenitrile (134 mg, 0.771 mmol) and K₂CO₃ (107 mg, 0.771 mmol) were suspended in DMF (3.4 mL) and stirred at room temperature overnight. The mixture was diluted with EtOAc and washed with water. The organic phase was dried over MgSO₄, filtered, and concentrated in vacuo. The crude mixture was subjected to silica gel flash column chromatography (0-100% EtOAc/hexanes) to afford the title compound. MS (m/z) 460.90, 462.02 [M+H]+. ¹H NMR (400 MHz, Acetonitrile-d3) δ 7.46 (d, J=16.7 Hz, 1H), 7.36 (s, 2H), 7.30 (d, J=8.6 Hz, 2H), 6.93 (d, J=8.6 Hz, 2H), 6.07 (d, J=16.7 Hz, 1H), 4.74 (s, 2H), 4.41 (s, 2H), 3.78 (s, 3H), 2.09 (s, 6H).

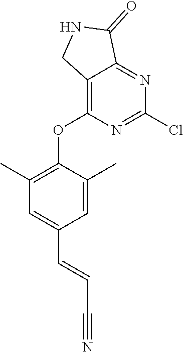 [SMILES: Cc1cc(/C=C/C#N)cc(C)c1Oc1nc(Cl)nc2c1CNC2=O]

### (E)-3-(4-((2-chloro-7-oxo-6,7-dihydro-5H-pyrrolo[3,4-d]pyrimidin-4-yl)oxy)-3,5-dimethylphenyl)acrylonitrile—Intermediate 153b

Intermediate 153a (10.0 mg, 0.0217 mmol) was suspended in a 3:1 solution of MeCN/H₂O (0.2 mL) and the solution was cooled to 0° C. To the solution was added ceric ammonium nitrate (23.8 mg, 0.0434 mmol) and the mixture was stirred at 0° C. for 1.25 h, at which point additional ceric ammonium nitrate (23.8 mg, 0.0434 mmol) was added. After stirring an additional 3.5 h, the mixture was diluted with EtOAc and washed twice with water. The organic phase was dried over MgSO₄, filtered, and concentrated in vacuo. The resulting crude mixture was subjected to silica gel flash column chromatography (0-20% MeOH/DCM) to afford the title compound. MS (m/z) 341.13 [M+H]+.

### (E)-3-((4-(4-(2-cyanovinyl)-2,6-dimethylphenoxy)-7-oxo-6,7-dihydro-5H-pyrrolo[3,4-d]pyrimidin-2-yl)amino)bicyclo[1.1.1]pentane-1-carbonitrile—Compound 153

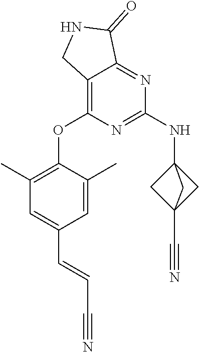 [SMILES: Cc1cc(/C=C/C#N)cc(C)c1Oc1nc(NC23CC(C#N)(C2)C3)nc2c1CNC2=O]

Compound 153 was prepared using the procedure described for the synthesis of Compound 4, using Intermediate 153b (8.2 mg, 0.024 mmol) instead of Intermediate 4a to afford the title product. MS (m/z) 413.24 [M+H]+. 1H NMR (400 MHz, Acetonitrile-d3) δ 7.48 (d, J=16.8 Hz, 1H), 7.40 (s, 2H), 7.18 (s, 1H), 6.56 (s, 1H), 6.07 (d, J=16.7 Hz, 1H), 4.38 (d, J=1.3 Hz, 2H), 2.11 (s, 12H).

### Example 154

### (E)-3-(3,5-dimethyl-4-((3-methyl-6-((3-(oxetan-3-yl)bicyclo[1.1.1]pentan-1-yl)amino)-1H-pyrazolo[3,4-d]pyrimidin-4-yl)oxy)phenyl)acrylonitrile—Compound 154

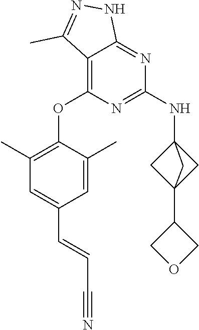 [SMILES: Cc1cc(/C=C/C#N)cc(C)c1Oc1nc(NC23CC(C4COC4)(C2)C3)nc2[nH]nc(C)c12]

Intermediate 119a (15.0 mg, 0.0441 mmol), 3-(oxetan-3-yl)bicyclo[1.1.1]pentan-1-amine (24.6 mg, 0.177 mmol), and DIPEA (23 μL, 0.13 mmol) were suspended in NMP (0.44 mL) and the solution was heated to 145° C. for 1.5 h. The solution was allowed to cool to room temperature at which point the mixture was diluted with DMF, filtered, and subjected to preparative HPLC (30-100% MeCN/H₂O) and lyophilized to afford the title product. MS (m/z) 443.17 [M+H]+. 1H NMR (400 MHz, Acetonitrile-d3) δ 10.54 (s, 1H), 7.44 (d, J=16.7 Hz, 1H), 7.37 (s, 2H), 6.12 (s, 1H), 6.03 (d, J=16.8 Hz, 1H), 4.54 (dd, J=7.9, 5.7 Hz, 2H), 4.15-4.02 (m, 2H), 2.99-2.88 (m, 1H), 2.55 (s, 3H), 2.14 (m, 12H). BCP/diMe peaks are under H₂O peak.

### Example 155

### (E)-3-(4-((2-((3-(2,2-difluoroethyl)bicyclo[1.1.1]pentan-1-yl)amino)-7-(4-methoxybenzyl)-7H-purin-6-yl)oxy)-3,5-dimethylphenyl)acrylonitrile—Intermediate 155a

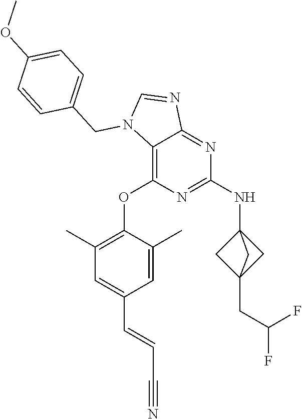 [SMILES: COc1ccc(Cn2cnc3nc(NC45CC(CC(F)F)(C4)C5)nc(Oc4c(C)cc(/C=C/C#N)cc4C)c32)cc1]

(E)-3-(4-((2-chloro-7-(4-methoxybenzyl)-7H-purin-6-yl)oxy)-3,5-dimethylphenyl)acrylonitrile (30 mg, 0.0673 mmol), 3-(2,2-difluoroethyl)bicyclo[1.1.1]pentan-1-amine; hydrochloride (14.8 mg, 0.807 mmol), cesium carbonate (43.8 mg, 0.135 mmol), Pd(PPh₃)₂Cl₂ (4.72 mg, 6.73 μM), and rac-BINAP (3.96 mg, 6.73 μM) were charged to a vial. The vial was evacuated and backfilled with Ar and 1,4-dioxane (0.5 mL) was added to the vial which was then sealed. The mixture was stirred at 120° C. for 1 h then 140° C. for 5 h and then allowed to cool to room temperature. The mixture was passed over a plug of celite with DCM then a 1:1 EtOAc/MeCN mixture. The filtrate was concentrated in vacuo and used directly in the next transformation. MS (m/z) 557.27 [M+H]+.

### (E)-3-(4-((2-((3-(2,2-difluoroethyl)bicyclo[1.1.1]pentan-1-yl)amino)-7H-purin-6-yl)oxy)-3,5-dimethylphenyl)acrylonitrile—Compound 155

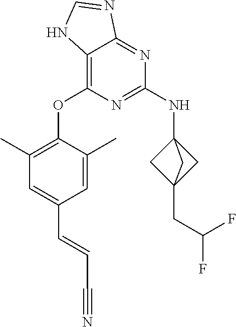 [SMILES: Cc1cc(/C=C/C#N)cc(C)c1Oc1nc(NC23CC(CC(F)F)(C2)C3)nc2nc[nH]c12]

Intermediate 155a (35.0 mg, 0.0785 mmol) was suspended in TFA (1 mL) and the solution was heated to 40° C. for 23 h then cooled to room temperature and concentrated in vacuo. The resulting residue was dissolved in EtOAc (3 mL) and washed with NaHCO₃ (1×3 mL). The combined organic layers were concentrated in vacuo and subjected to preparative HPLC (0-100% MeCN/H₂O) and lyophilized to afford the title compound. MS (m/z) 437.19 [M+H]+. ¹H NMR (400 MHz, DMSO-d6) δ 12.69 (s, 1H), 7.99 (s, 1H), 7.61 (d, J=16.7 Hz, 1H), 7.50 (s, 2H), 7.44 (s, 1H), 6.42 (d, J=16.7 Hz, 1H), 5.76 (t, J=57.2 Hz, 1H), 2.08 (s, 6H), 1.90 (m, 2H), 1.57 (br s, 6H).

### Example 176

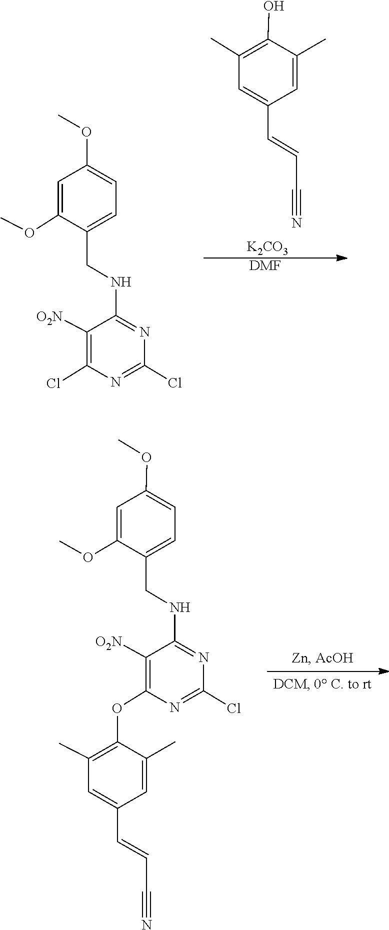

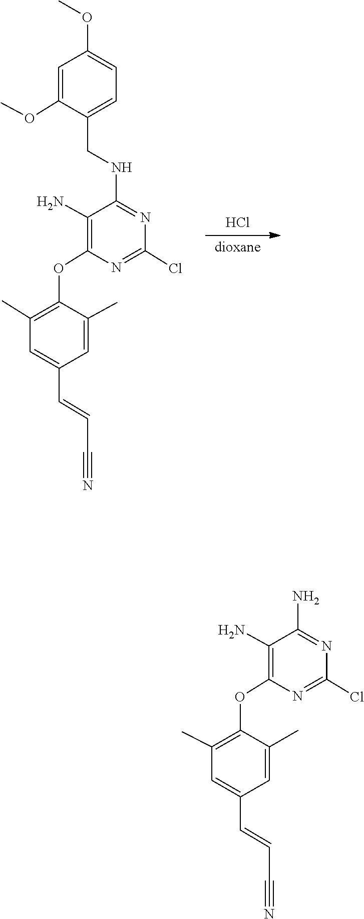

### Step 1, preparation of (E)-3-(4-((2-chloro-6-((2,4-dimethoxybenzyl)amino)-5-nitropyrimidin-4-yl)oxy)-3,5-dimethylphenyl)acrylonitrile (Intermediate 176a)

A mixture of 2,6-dichloro-N-(2,4-dimethoxybenzyl)-5-nitropyrimidin-4-amine (1490 mg, 8.60 mmol), (E)-3-(4-hydroxy-3,5-dimethyl-phenyl)prop-2-enenitrile (1490 mg, 8.60 mmol), and potassium carbonate (5945 mg, 43.0 mmol) in DMF (45 mL) was stirred at room temperature overnight. The mixture was then diluted with EtOAc and washed twice with brine. The organic phase was dried over MgSO₄, filtered, and concentrated in vacuo. The resulting residue was purified by silica gel chromatography (0-100% EtOAc/hexanes) to afford the title compound. MS (m/z) 496.0 [M+H]+.

### Step 2, preparation of (E)-3-(4-((5-amino-2-chloro-6-((2,4-dimethoxybenzyl)amino)pyrimidin-4-yl)oxy)-3,5-dimethylphenyl)acrylonitrile (Intermediate 176b)

To a solution of (E)-3-(4-((2-chloro-6-((2,4-dimethoxybenzyl)amino)-5-nitropyrimidin-4-yl)oxy)-3,5-dimethylphenyl)acrylonitrile (2.0 g, 4.03 mmol) in DCM (40 mL) at 0° C. was added zinc dust (3.96 g, 60.5 mmol), followed by acetic acid (1.73 mL, 30.2 mmol) (steadily dropwise). The mixture was stirred for 1 h, then filtered through celite to remove zinc, and the filtrate concentrated in vacuo. The resulting residue was diluted with EtOAc, then basified with sat NaHCO₃, layers separated, and the aqueous phase extracted with additional EtOAc. The combined organics were dried over MgSO₄, filtered, and concentrated in vacuo. The resulting residue was used without further purification. MS (m/z) 465.9 [M+H]+.

### Step 3, preparation of (E)-3-(4-((5,6-diamino-2-chloropyrimidin-4-yl)oxy)-3,5-dimethylphenyl)acrylonitrile (Intermediate 176c)

To a solution of (E)-3-(4-((5-amino-2-chloro-6-((2,4-dimethoxybenzyl)amino)pyrimidin-4-yl)oxy)-3,5-dimethylphenyl)acrylonitrile (1.46 g, 3.13 mmol) in dioxane (10.0 mL) was added hydrochloric acid (4.00 mol/L, 15.7 mL, 62.7 mmol), and the mixture stirred at rt for 2 h. The mixture was then concentrated in vacuo, partitioned between EtOAc and sat NaHCO₃ solution, the layers separated, and the aqueous phase extracted with additional EtOAc. Combined organics were dried over MgSO₄, filtered, and concentrated in vacuo. Used without further purification. MS (m/z): 316.0 [M+H]+.

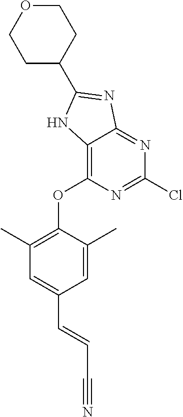 [SMILES: Cc1cc(/C=C/C#N)cc(C)c1Oc1nc(Cl)nc2nc(C3CCOCC3)[nH]c12]

(E)-3-(4-((2-chloro-8-(tetrahydro-2H-pyran-4-yl)-7H-purin-6-yl)oxy)-3,5-dimethylphenyl)acrylonitrile (Intermediate 176d) was prepared using the procedure described for the synthesis of (E)-3-((6-(4-(2-cyanovinyl)-2,6-dimethylphenoxy)-8-(tetrahydro-2H-pyran-4-yl)-7H-purin-2-yl)amino)bicyclo[1.1.1]pentane-1-carbonitrile (Compound 52) with the following modifications: (E)-3-(4-((5,6-diamino-2-chloropyrimidin-4-yl)oxy)-3,5-dimethylphenyl)acrylonitrile was used in place of 3-[[4,5-diamino-6-[4-[(E)-2-cyanovinyl]-2,6-dimethyl-phenoxy]pyrimidin-2-yl]amino]bicyclo[1.1.1]pentane-1-carbonitrile. MS (m/z) 409.9 [M+H]+.

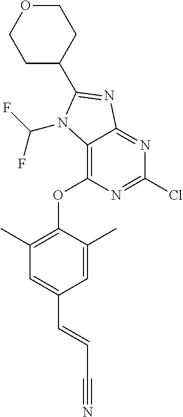 [SMILES: Cc1cc(/C=C/C#N)cc(C)c1Oc1nc(Cl)nc2nc(C3CCOCC3)n(C(F)F)c12]

(E)-3-(4-((2-chloro-7-(difluoromethyl)-8-(tetrahydro-2H-pyran-4-yl)-7H-purin-6-yl)oxy)-3,5-dimethylphenyl)acrylonitrile (Intermediate 176e) was prepared using the procedure described for the synthesis of (E)-3-(4-((2-chloro-7-(difluoromethyl)-8-methyl-7H-purin-6-yl)oxy)-3,5-dimethylphenyl)acrylonitrile (Intermediate 13d) with the following modifications: (E)-3-(4-((2-chloro-8-(tetrahydro-2H-pyran-4-yl)-7H-purin-6-yl)oxy)-3,5-dimethylphenyl)acrylonitrile was used in place of (E)-3-(4-((2-chloro-8-methyl-7H-purin-6-yl)oxy)-3,5-dimethylphenyl)acrylonitrile. MS (m/z) 459.9 [M+H]+.

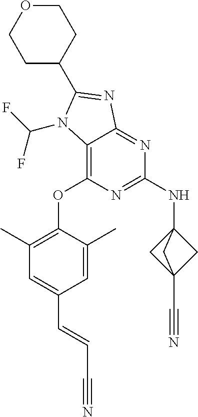 [SMILES: Cc1cc(/C=C/C#N)cc(C)c1Oc1nc(NC23CC(C#N)(C2)C3)nc2nc(C3CCOCC3)n(C(F)F)c12]

(E)-3-((6-(4-(2-cyanovinyl)-2,6-dimethylphenoxy)-7-(difluoromethyl)-8-(tetrahydro-2H-pyran-4-yl)-7H-purin-2-yl)amino)bicyclo[1.1.1]pentane-1-carbonitrile (Compound 176) was prepared using the procedure described for the synthesis of (E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile (Compound 13) with the following modifications: (E)-3-(4-((2-chloro-7-(difluoromethyl)-8-(tetrahydro-2H-pyran-4-yl)-7H-purin-6-yl)oxy)-3,5-dimethylphenyl)acrylonitrile was used in place of (E)-3-(4-((2-chloro-7-(difluoromethyl)-8-methyl-7H-purin-6-yl)oxy)-3,5-dimethylphenyl)acrylonitrile. MS (m/z) 531.9 [M+H]+. ¹H NMR (400 MHz, Acetonitrile-d3) δ 7.82 (t, J=57.8 Hz, 1H), 7.51 (d, J=16.8 Hz, 1H), 7.43 (s, 2H), 6.20 (s, 1H), 6.10 (d, J=16.7 Hz, 1H), 4.09-3.97 (m, 2H), 3.62-3.52 (m, 2H), 3.42-3.32 (m, 1H), 2.15-2.13 (m, 12H). ¹⁹F NMR (376 MHz, Acetonitrile-d3) δ−92.61, −92.76.

### Example 177

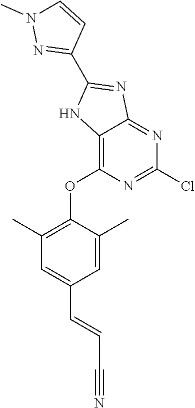 [SMILES: Cc1cc(/C=C/C#N)cc(C)c1Oc1nc(Cl)nc2nc(-c3ccn(C)n3)[nH]c12]

(E)-3-(4-((2-chloro-8-(1-methyl-1H-pyrazol-3-yl)-7H1-purin-6-yl)oxy)-3,5-dim ethylphenyl)acrylonitrile (Intermediate 177a) was prepared using the procedure described for the synthesis of (E)-3-((6-(4-(2-cyanovinyl)-2,6-dimethylphenoxy)-8-(tetrahydro-211-pyran-4-yl)-711-purin-2-yl)amino)bicyclo[1.1.1]pentane-1-carbonitrile (Compound 52) with the following modifications: (E)-3-(4-((5,6-diamnino-2-chloropyrimnidin-4-yl)oxy)-3,5-dimnethylphenyl)acrylonitrile and 1-mnethylpyrazole-3-carbaldehyde were used in place of 3-[[4, 5-diamnino-6-[4-[(E)-2-cyanovinyl]-2,6-dimnethyl-phenoxy]pyrimnidin-2-yl]ainino]bicyclo[1.1.1]pentane-1-carbonitrile and tetrahydropyran-4-carbaldehyde, respectively. MS (m/z) 405.8 [M+H]+.

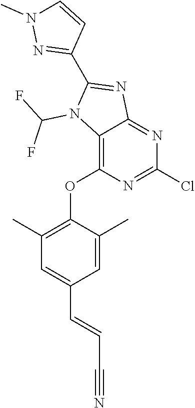 [SMILES: Cc1cc(/C=C/C#N)cc(C)c1Oc1nc(Cl)nc2nc(-c3ccn(C)n3)n(C(F)F)c12]

(E)-3-(4-((2-chloro-7-(difluoromethyl)-8-(1-methyl-1H-pyrazol-3-yl)-7H-purin-6-yl)oxy)-3,5-dimethylphenyl)acrylonitrile (Intermediate 177b) was prepared using the procedure described for the synthesis of (E)-3-(4-((2-chloro-7-(difluoromethyl)-8-methyl-7H-purin-6-yl)oxy)-3,5-dimethylphenyl)acrylonitrile (Intermediate 13d) with the following modifications: (E)-3-(4-((2-chloro-8-(1-methyl-1H-pyrazol-3-yl)-7H-purin-6-yl)oxy)-3,5-dimethylphenyl)acrylonitrile was used in place of (E)-3-(4-((2-chloro-8-methyl-7H-purin-6-yl)oxy)-3,5-dimethylphenyl)acrylonitrile. MS (m/z) 455.8 [M+H]+.

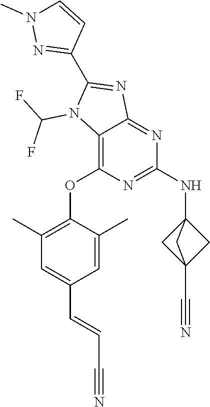 [SMILES: Cc1cc(/C=C/C#N)cc(C)c1Oc1nc(NC23CC(C#N)(C2)C3)nc2nc(-c3ccn(C)n3)n(C(F)F)c12]

(E)-3-((6-(4-(2-cyanovinyl)-2,6-dimethylphenoxy)-7-(difluoromethyl)-8-(1-methyl-1H-pyrazol-3-yl)-7H-purin-2-yl)amino)bicyclo[1.1.1]pentane-1-carbonitrile (Compound 177) was prepared using the procedure described for the synthesis of (E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile (Compound 13) with the following modifications: (E)-3-(4-((2-chloro-7-(difluoromethyl)-8-(1-methyl-1H-pyrazol-3-yl)-7H-purin-6-yl)oxy)-3,5-dimethylphenyl)acrylonitrile was used in place of (E)-3-(4-((2-chloro-7-(difluoromethyl)-8-methyl-7H-purin-6-yl)oxy)-3,5-dimethylphenyl)acrylonitrile. MS (m/z) 527.9 [M+H]+. ¹H NMR (400 MHz, DMSO-d6) δ 8.82 (t, J=58.0 Hz, 1H), 8.09-7.98 (m, 2H), 7.64 (d, J=16.6 Hz, 1H), 7.57 (s, 2H), 7.05 (d, J=2.3 Hz, 1H), 6.46 (d, J=16.6 Hz, 1H), 4.03 (s, 3H), 2.17-1.88 (m, 12H).

### Example 178

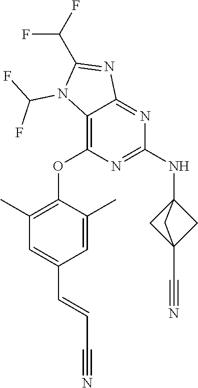 [SMILES: Cc1cc(/C=C/C#N)cc(C)c1Oc1nc(NC23CC(C#N)(C2)C3)nc2nc(C(F)F)n(C(F)F)c12]

(E)-3-((6-(4-(2-cyanovinyl)-2,6-dimethylphenoxy)-7,8-bis(difluoromethyl)-7H-purin-2-yl)amino)bicyclo[1.1.1]pentane-1-carbonitrile was prepared using the procedure described for the synthesis of (E)-3-(4-((2-chloro-7-(difluoromethyl)-8-methyl-7H-purin-6-yl)oxy)-3,5-dimethylphenyl)acrylonitrile (Intermediate 13d) with the following modifications: (E)-3-((6-(4-(2-cyanovinyl)-2,6-dimethylphenoxy)-8-(difluoromethyl)-7H-purin-2-yl)amino)bicyclo[1.1.1]pentane-1-carbonitrile was used in place of (E)-3-(4-((2-chloro-8-methyl-7H-purin-6-yl)oxy)-3,5-dimethylphenyl)acrylonitrile. MS (m/z) 497.9 [M+H]+. ¹H NMR (400 MHz, DMSO-d6) δ 8.39-8.02 (m, 2H), 7.71-7.50 (m, 3H), 6.46 (d, J=16.8 Hz, 1H), 2.22-1.81 (m, 12H). ¹⁹F NMR (376 MHz, DMSO-d6) δ −91.76 (d, J=57.2 Hz), −117.67 (d, J=50.4 Hz).

### Example 179

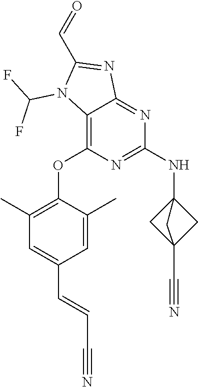 [SMILES: Cc1cc(/C=C/C#N)cc(C)c1Oc1nc(NC23CC(C#N)(C2)C3)nc2nc(C=O)n(C(F)F)c12]

(E)-3-((6-(4-(2-cyanovinyl)-2,6-dimethylphenoxy)-7-(difluoromethyl)-8-formyl-7H-purin-2-yl)amino)bicyclo[1.1.1]pentane-1-carbonitrile (Intermediate 179a) was prepared as follows: To a mixture of (E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile (Compound 13) (15.0 mg, 0.0325 mmol) in dioxane (0.3 mL) was added selenium dioxide (7.2 mg, 0.0650 mmol), and the mixture was stirred at 80° C. overnight. The mixture was then filtered through celite, washing with EtOAc. The filtrate was concentrated in vacuo and the resulting residue was used without further purification. MS (m/z) 493.9 [M+H₂O+H]+.

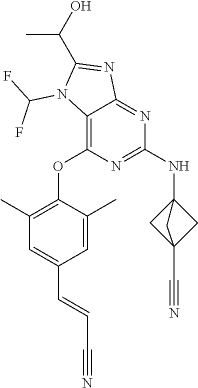 [SMILES: Cc1cc(/C=C/C#N)cc(C)c1Oc1nc(NC23CC(C#N)(C2)C3)nc2nc(C(C)O)n(C(F)F)c12]

(E)-3-((6-(4-(2-cyanovinyl)-2,6-dimethylphenoxy)-7-(difluoromethyl)-8-(1-hydroxyethyl)-7H-purin-2-yl)amino)bicyclo[1.1.1]pentane-1-carbonitrile (Compound 179) was prepared as follows: To a mixture of (E)-3-((6-(4-(2-cyanovinyl)-2,6-dimethylphenoxy)-7-(difluoromethyl)-8-formyl-7H-purin-2-yl)amino)bicyclo[1.1.1]pentane-1-carbonitrile (15.5 mg, 0.0325 mmol) in THE (0.50 mL) at 0° C. was added methylmagnesium bromide (3.0 M, 0.0108 mL, 0.0325 mmol), and the mixture was stirred for 1.5 h, allowing to warm to rt. H₂O was then added, and the mixture diluted with DMF and purified by preparative HPLC (0-100% MeCN/water) to afford the title compound. MS (m/z) 491.9 [M+H]+. ¹H NMR (400 MHz, DMSO-d6) δ 8.22 (t, J=57.5 Hz, 1H), 7.99 (s, 1H), 7.64 (d, J=16.6 Hz, 1H), 7.57 (s, 2H), 6.45 (d, J=16.7 Hz, 1H), 6.20 (s, 1H), 5.26-5.12 (m, 1H), 2.18-1.92 (m, 12H), 1.60 (d, J=6.6 Hz, 3H). ¹⁹F NMR (376 MHz, DMSO-d6) δ−91.91-−92.59 (m).

### Example 180

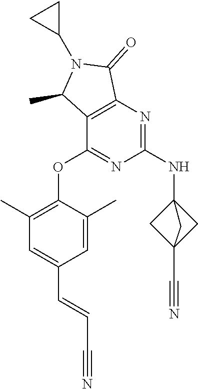 [SMILES: Cc1cc(/C=C/C#N)cc(C)c1Oc1nc(NC23CC(C#N)(C2)C3)nc2c1[C@@H](C)N(C1CC1)C2=O]

(R,E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl)amino)bicyclo[1.1.1]pentane-1-carbonitrile (Compound 180) was prepared using the procedure described for the synthesis of 3-[[(5R)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[1.1.1]pentane-1-carbonitrile (Compound 137) with the following modifications: (E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl)amino)bicyclo[1.1.1]pentane-1-carbonitrile was used in place of (E)-3-((4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile. MS (m/z) 466.9 [M+H]+. ¹H NMR (400 MHz, DMSO-d6) δ 8.53 (s, 1H), 7.63 (d, J=16.6 Hz, 1H), 7.56 (s, 2H), 6.44 (d, J=16.6 Hz, 1H), 4.76-4.64 (m, 1H), 2.72-2.64 (m, 1H), 2.08 (d, J=4.4 Hz, 6H), 1.96 (s, 6H), 1.58 (d, J=6.5 Hz, 3H), 1.00-0.89 (m, 2H), 0.82-0.71 (m, 2H).

### Example 181

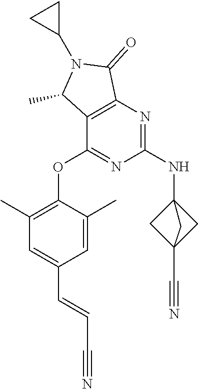 [SMILES: Cc1cc(/C=C/C#N)cc(C)c1Oc1nc(NC23CC(C#N)(C2)C3)nc2c1[C@H](C)N(C1CC1)C2=O]

(S,E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl)amino)bicyclo[1.1.1]pentane-1-carbonitrile (Compound 181) was prepared using the procedure described for the synthesis of 3-[[(5R)-4-[4-[(E)-2-cyanovinyl]-2,6-dimethyl-phenoxy]-5-methyl-7-oxo-5,6-dihydropyrrolo[3,4-d]pyrimidin-2-yl]amino]bicyclo[1.1.1]pentane-1-carbonitrile (Compound 137) with the following modifications: (E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl)amino)bicyclo[1.1.1]pentane-1-carbonitrile was used in place of (E)-3-((4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile. MS (m/z) 467.0 [M+H]+. ¹H NMR (400 MHz, DMSO-d6) δ 8.54 (d, J=12.9 Hz, 1H), 7.63 (d, J=16.7 Hz, 1H), 7.56 (s, 2H), 6.44 (d, J=16.7 Hz, 1H), 4.75-4.65 (m, 1H), 2.73-2.65 (m, 1H), 2.08 (d, J=4.5 Hz, 6H), 1.96 (s, 6H), 1.58 (d, J=6.5 Hz, 3H), 0.98-0.89 (m, 2H), 0.81-0.72 (m, 2H).

### Example 182

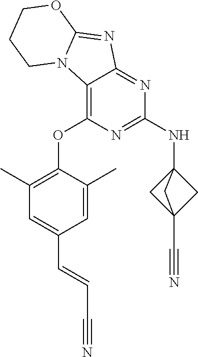 [SMILES: Cc1cc(/C=C/C#N)cc(C)c1Oc1nc(NC23CC(C#N)(C2)C3)nc2nc3n(c12)CCCO3]

(E)-3-((4-(4-(2-cyanovinyl)-2,6-dimethylphenoxy)-7,8-dihydro-6H-[1,3]oxazino[2,3-f]purin-2-yl)amino)bicyclo[1.1.1]pentane-1-carbonitrile (Compound 182) was prepared as follows: To a solution of 3-[[6-[4-[(E)-2-cyanovinyl]-2,6-dimethyl-phenoxy]-8-oxo-7,9-dihydropurin-2-yl]amino]bicyclo[1.1.1]pentane-1-carbonitrile (20.0 mg, 0.0484 mmol) and potassium carbonate (0.0201 g, 0.145 mmol) in DMF (0.500 mL) was added 1,3-Diiodopropane (0.00667 mL, 0.0581 mmol), and the mixture was heated to 50° C. for 1 h. The mixture was diluted with DMF/H₂O and purified by preparative HPLC (0-100% MeCN/water) to afford the title compound (eluted as isomer 1). MS (m/z) 453.9 [M+H]+. ¹H NMR (400 MHz, Acetonitrile-d3) δ 7.51 (d, J=16.7 Hz, 1H), 7.41 (s, 2H), 6.09 (d, J=16.6 Hz, 1H), 5.90 (s, 1H), 4.59-4.51 (m, 2H), 4.36 (t, J=6.1 Hz, 2H), 2.35-2.28 (m, 2H), 2.14-2.09 (m, 12H).

### Example 183

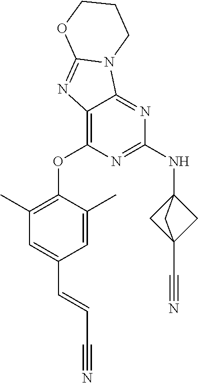 [SMILES: Cc1cc(/C=C/C#N)cc(C)c1Oc1nc(NC23CC(C#N)(C2)C3)nc2c1nc1n2CCCO1]

(E)-3-((4-(4-(2-cyanovinyl)-2,6-dimethylphenoxy)-8,9-dihydro-7H-[1,3]oxazino[3,2-e]purin-2-yl)amino)bicyclo[1.1.1]pentane-1-carbonitrile (Compound 183) was prepared using the procedure described for the synthesis of (E)-3-((4-(4-(2-cyanovinyl)-2,6-dimethylphenoxy)-7,8-dihydro-6H-[1,3]oxazino[2,3-f]purin-2-yl)amino)bicyclo[1.1.1]pentane-1-carbonitrile (Compound 182) (eluted as isomer 2). MS (m/z) 453.9 [M+H]+. ¹H NMR (400 MHz, Acetonitrile-d3) δ 7.47 (d, J=16.7 Hz, 1H), 7.38-7.35 (m, 2H), 6.05 (d, J=16.7 Hz, 1H), 5.94 (s, 1H), 4.51-4.45 (m, 2H), 3.99 (t, J=6.2 Hz, 2H), 2.27-2.18 (m, 2H), 2.12-2.05 (m, 12H).

### Example 184

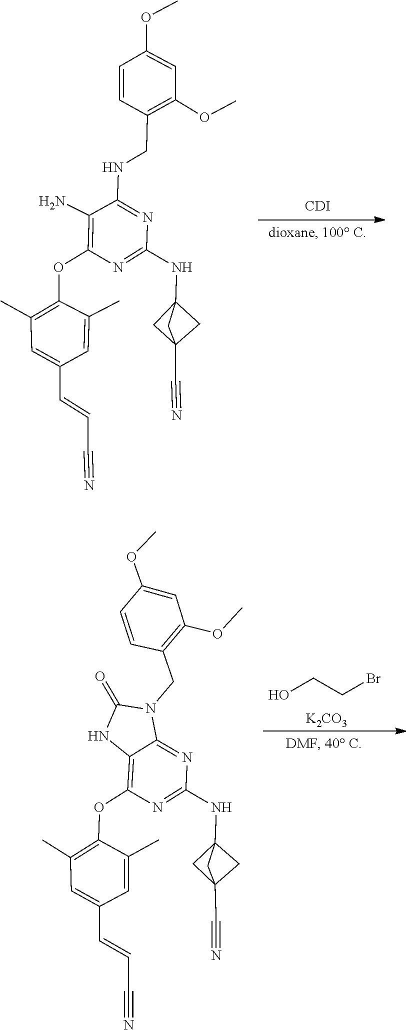

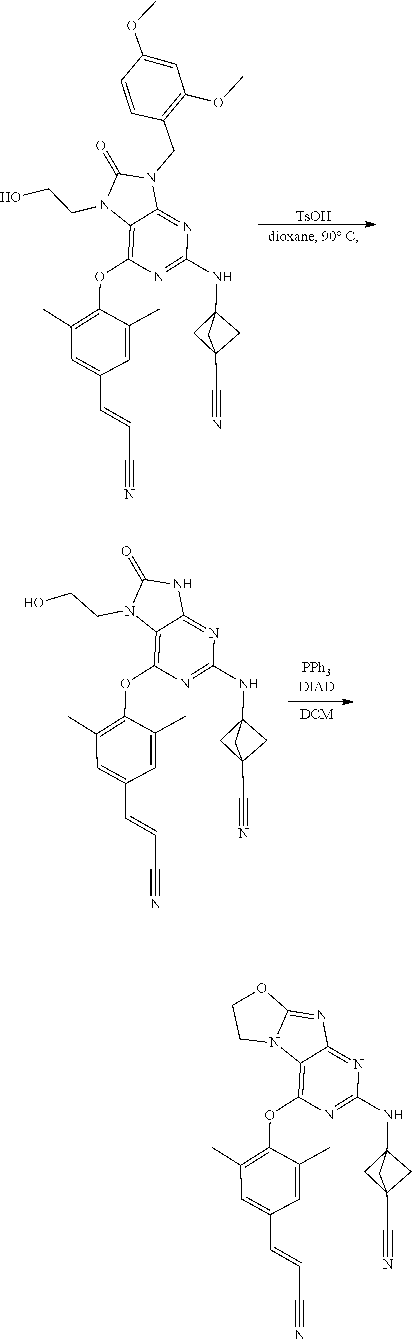 [SMILES: COc1ccc(Cn2c(=O)n(CCO)c3c(Oc4c(C)cc(/C=C/C#N)cc4C)nc(NC45CC(C#N)(C4)C5)nc32)c(OC)c1 ; "TsOH" ; "dioxane, 90° C," ; Cc1cc(/C=C/C#N)cc(C)c1Oc1nc(NC23CC(C#N)(C2)C3)nc2[nH]c(=O)n(CCO)c12 ; Cc1cc(/C=C/C#N)cc(C)c1Oc1nc(NC23CC(C#N)(C2)C3)nc2nc3n(c12)CCO3]

### Step 1, preparation of (E)-3-((6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile (Intermediate 184a)

A mixture of (E)-3-((5-amino-4-(4-(2-cyanovinyl)-2,6-dimethylphenoxy)-6-((2,4-dimethoxybenzyl)amino)pyrimidin-2-yl)amino)bicyclo[1.1.1]pentane-1-carbonitrile (750 mg, 1.40 mmol) and di(imidazol-1-yl)methanone (0.339 g, 2.09 mmol) in dioxane (10.0 mL) was heated to 100° C. overnight. The mixture was then concentrated in vacuo and the resulting residue was purified by silica gel chromatography (0-100% EtOAc/hexanes) to afford the title compound. MS (m/z): 563.9 [M+H]+.

### Step 2, preparation of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b)

To a mixture of (E)-3-((6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile (10.0 mg, 0.0177 mmol) and potassium carbonate (0.00736 g, 0.0532 mmol) in DMF (0.250 mL) was added 2-bromoethanol (0.00126 mL, 0.0177 mmol), and the mixture was stirred at 40° C. overnight. The mixture was diluted with EtOAc, washed twice with brine, and the organics were then dried over MgSO₄, filtered, and concentrated in vacuo. The resulting residue was used without further purification. MS (m/z): 607.9 [M+H]+.

### Step 3, preparation of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c)

To a mixture of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (39.0 mg, 0.0642 mmol) in dioxane (0.650 mL) was added trifluoromethanesulfonic acid (0.0114 mL, 0.128 mmol). The mixture was then heated to 90° C. for 1 h. The reaction mixture was concentrated in vacuo, then diluted with EtOAc and basified with sat. NaHCO₃ solution. The layers were separated and the aqueous phase extracted with additional EtOAc. Combined organics were dried over MgSO₄, filtered, and concentrated in vacuo. The resulting residue was used without further purification. MS (m/z): 457.9 [M+H]+.

### Step 4, preparation of (E)-3-((4-(4-(2-cyanovinyl)-2,6-dimethylphenoxy)-6,7-dihydrooxazolo[2,3-f]purin-2-yl)amino)bicyclo[1.1.1]pentane-1-carbonitrile (Compound 184)

To a solution of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (29.0 mg, 0.0634 mmol) and triphenylphosphine (0.0499 g, 0.190 mmol) in DCM (1.0 mL) was added diisopropyl azodicarboxylate (0.0374 mL, 0.190 mmol) steadily dropwise. The mixture was stirred at rt overnight, then concentrated in vacuo and then diluted with DMF/H₂O and purified by preparative HPLC (0-100% MeCN/water) to afford the title compound. MS (m/z) 439.9 [M+H]+. ¹H NMR (400 MHz, DMSO-d6) δ 7.66-7.58 (m, 2H), 7.53 (s, 2H), 6.43 (d, J=16.7 Hz, 1H), 5.18 (dd, J=8.7, 6.9 Hz, 2H), 4.50-4.35 (m, 2H), 2.18-1.95 (m, 12H).

### Example 185

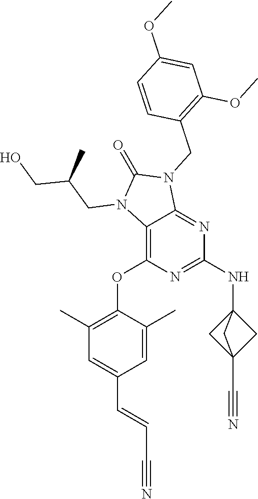 [SMILES: COc1ccc(Cn2c(=O)n(C[C@H](C)CO)c3c(Oc4c(C)cc(/C=C/C#N)cc4C)nc(NC45CC(C#N)(C4)C5)nc32)c(OC)c1]

(S,E)-3-((6-(4-(2-cyanovinyl)-2,6-dimethylphenoxy)-9-(2,4-dimethoxybenzyl)-7-(3-hydroxy-2-methylpropyl)-8-oxo-8,9-dihydro-7H-purin-2-yl)amino)bicyclo[1.1.1]pentane-1-carbonitrile (Intermediate 185a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: (R)-3-bromo-2-methyl-propan-1-ol was used in place of 2-bromoethanol. MS (m/z) 635.9 [M+H]+.

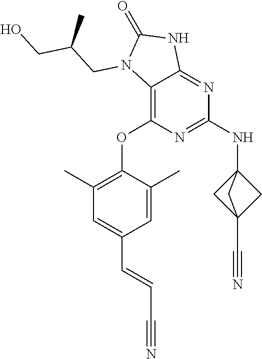 [SMILES: Cc1cc(/C=C/C#N)cc(C)c1Oc1nc(NC23CC(C#N)(C2)C3)nc2[nH]c(=O)n(C[C@H](C)CO)c12]

(S,E)-3-((6-(4-(2-cyanovinyl)-2,6-dimethylphenoxy)-7-(3-hydroxy-2-methylpropyl)-8-oxo-8,9-dihydro-7H-purin-2-yl)amino)bicyclo[1.1.1]pentane-1-carbonitrile (Intermediate 185b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (S,E)-3-((6-(4-(2-cyanovinyl)-2,6-dimethylphenoxy)-9-(2,4-dimethoxybenzyl)-7-(3-hydroxy-2-methylpropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 486.0 [M+H]+.

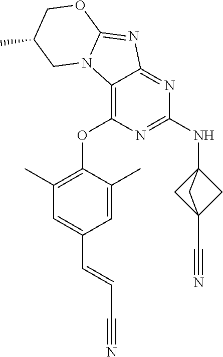 [SMILES: Cc1cc(/C=C/C#N)cc(C)c1Oc1nc(NC23CC(C#N)(C2)C3)nc2nc3n(c12)C[C@H](C)CO3]

(S,E)-3-((4-(4-(2-cyanovinyl)-2,6-dimethylphenoxy)-7-methyl-7,8-dihydro-6H-[1,3]oxazino[2,3-f]purin-2-yl)amino)bicyclo[1.1.1]pentane-1-carbonitrile (Compound 185) was prepared using the procedure described for the synthesis of (E)-3-((4-(4-(2-cyanovinyl)-2,6-dimethylphenoxy)-6,7-dihydrooxazolo[2,3-f]purin-2-yl)amino)bicyclo[1.1.1]pentane-1-carbonitrile (Compound 184) with the following modifications: (S,E)-3-((6-(4-(2-cyanovinyl)-2,6-dimethylphenoxy)-7-(3-hydroxy-2-methylpropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile. MS (m/z) 467.9 [M+H]+. ¹H NMR (400 MHz, DMSO-d6) δ 7.62 (d, J=16.7 Hz, 1H), 7.58-7.47 (m, 3H), 6.42 (d, J=16.7 Hz, 1H), 4.52 (dd, J=10.8, 3.4 Hz, 1H), 4.42 (dd, J=11.6, 5.4 Hz, 1H), 4.23 (t, J=10.3 Hz, 1H), 3.97-3.84 (m, 1H), 2.19-1.92 (m, 12H), 1.06 (d, J=6.8 Hz, 3H).

### Example 186

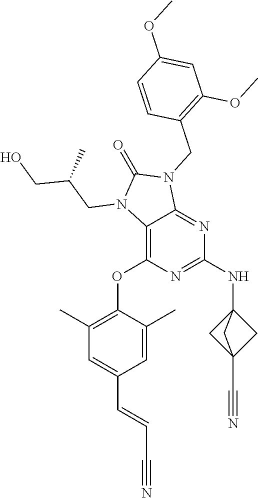 [SMILES: COc1ccc(Cn2c(=O)n(C[C@@H](C)CO)c3c(Oc4c(C)cc(/C=C/C#N)cc4C)nc(NC45CC(C#N)(C4)C5)nc32)c(OC)c1]

(R,E)-3-((6-(4-(2-cyanovinyl)-2,6-dimethylphenoxy)-9-(2,4-dimethoxybenzyl)-7-(3-hydroxy-2-methylpropyl)-8-oxo-8,9-dihydro-7H-purin-2-yl)amino)bicyclo[1.1.1]pentane-1-carbonitrile (Intermediate 186a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: (S)-3-bromo-2-methyl-propan-1-ol was used in place of 2-bromoethanol. MS (m/z) 635.9 [M+H]+.

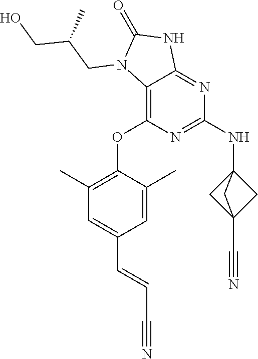 [SMILES: Cc1cc(/C=C/C#N)cc(C)c1Oc1nc(NC23CC(C#N)(C2)C3)nc2[nH]c(=O)n(C[C@@H](C)CO)c12]

(R,E)-3-((6-(4-(2-cyanovinyl)-2,6-dimethylphenoxy)-7-(3-hydroxy-2-methylpropyl)-8-oxo-8,9-dihydro-7H-purin-2-yl)amino)bicyclo[1.1.1]pentane-1-carbonitrile (Intermediate 186b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (R,E)-3-((6-(4-(2-cyanovinyl)-2,6-dimethylphenoxy)-9-(2,4-dimethoxybenzyl)-7-(3-hydroxy-2-methylpropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 486.0 [M+H]+.

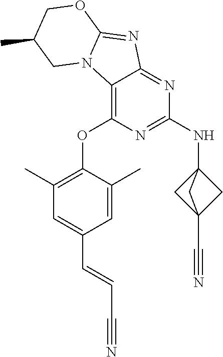 [SMILES: Cc1cc(/C=C/C#N)cc(C)c1Oc1nc(NC23CC(C#N)(C2)C3)nc2nc3n(c12)C[C@@H](C)CO3]

(R,E)-3-((4-(4-(2-cyanovinyl)-2,6-dimethylphenoxy)-7-methyl-7,8-dihydro-6H-[1,3]oxazino[2,3-f]purin-2-yl)amino)bicyclo[1.1.1]pentane-1-carbonitrile (Compound 186) was prepared using the procedure described for the synthesis of (E)-3-((4-(4-(2-cyanovinyl)-2,6-dimethylphenoxy)-6,7-dihydrooxazolo[2,3-f]purin-2-yl)amino)bicyclo[1.1.1]pentane-1-carbonitrile (Compound 184) with the following modifications: (R,E)-3-((6-(4-(2-cyanovinyl)-2,6-dimethylphenoxy)-7-(3-hydroxy-2-methylpropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile. MS (m/z) 467.9 [M+H]+. ¹H NMR (400 MHz, DMSO-d6) δ 7.62 (d, J=16.7 Hz, 1H), 7.56-7.49 (m, 3H), 6.43 (d, J=16.7 Hz, 1H), 4.57-4.48 (m, 1H), 4.47-4.37 (m, 1H), 4.23 (t, J=10.3 Hz, 1H), 3.94-3.86 (m, 1H), 2.18-1.89 (m, 12H), 1.06 (d, J=6.8 Hz, 3H).

### Example 187

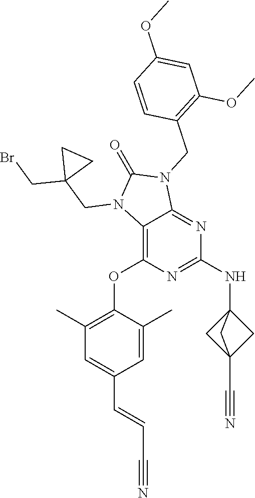 [SMILES: COc1ccc(Cn2c(=O)n(CC3(CBr)CC3)c3c(Oc4c(C)cc(/C=C/C#N)cc4C)nc(NC45CC(C#N)(C4)C5)nc32)c(OC)c1]

(E)-3-((7-((1-(bromomethyl)cyclopropyl)methyl)-6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile (Intermediate 187a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: 1,1-bis(bromomethyl)cyclopropane was used in place of 2-bromoethanol. MS (m/z) 709.8 [M+H]+.

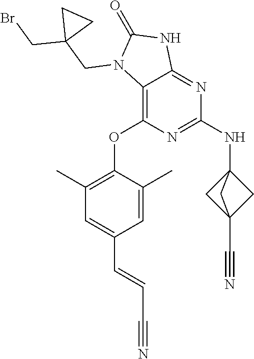 [SMILES: Cc1cc(/C=C/C#N)cc(C)c1Oc1nc(NC23CC(C#N)(C2)C3)nc2[nH]c(=O)n(CC3(CBr)CC3)c12]

(E)-3-((7-((1-(bromomethyl)cyclopropyl)methyl)-6-(4-(2-cyanovinyl)-2,6-dimethylphenoxy)-8-oxo-8,9-dihydro-7H-purin-2-yl)amino)bicyclo[1.1.1]pentane-1-carbonitrile (Intermediate 187b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-((7-((1-(bromomethyl)cyclopropyl)methyl)-6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 559.8 [M+H]+.

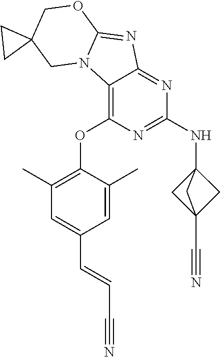 [SMILES: Cc1cc(/C=C/C#N)cc(C)c1Oc1nc(NC23CC(C#N)(C2)C3)nc2nc3n(c12)CC1(CC1)CO3]

(E)-3-((4′-(4-(2-cyanovinyl)-2,6-dimethylphenoxy)-6′H,8′H-spiro[cyclopropane-1,7′-[1,3]oxazino[2,3-f]purin]-2′-yl)amino)bicyclo[1.1.1]pentane-1-carbonitrile (Compound 187) was prepared as follows: A mixture of (E)-3-((7-((1-(bromomethyl)cyclopropyl)methyl)-6-(4-(2-cyanovinyl)-2,6-dimethylphenoxy)-8-oxo-8,9-dihydro-7H-purin-2-yl)amino)bicyclo[1.1.1]pentane-1-carbonitrile (20.5 mg, 0.0366 mmol) and potassium carbonate (0.0152 g, 0.110 mmol) in DMF (0.6 mL) was heated to 50° C. for 6 h. The mixture was diluted with DMF/H₂O and purified by preparative HPLC (0-100% MeCN/water) to afford the title compound. MS (m/z) 480.0 [M+H]+. ¹H NMR (400 MHz, DMSO-d6) δ 7.61 (d, J=16.7 Hz, 1H), 7.57-7.47 (m, 3H), 6.42 (d, J=16.7 Hz, 1H), 4.36 (s, 2H), 4.20 (s, 2H), 2.10 (s, 6H), 2.04 (s, 6H), 0.93-0.77 (m, 4H).

### Example 188

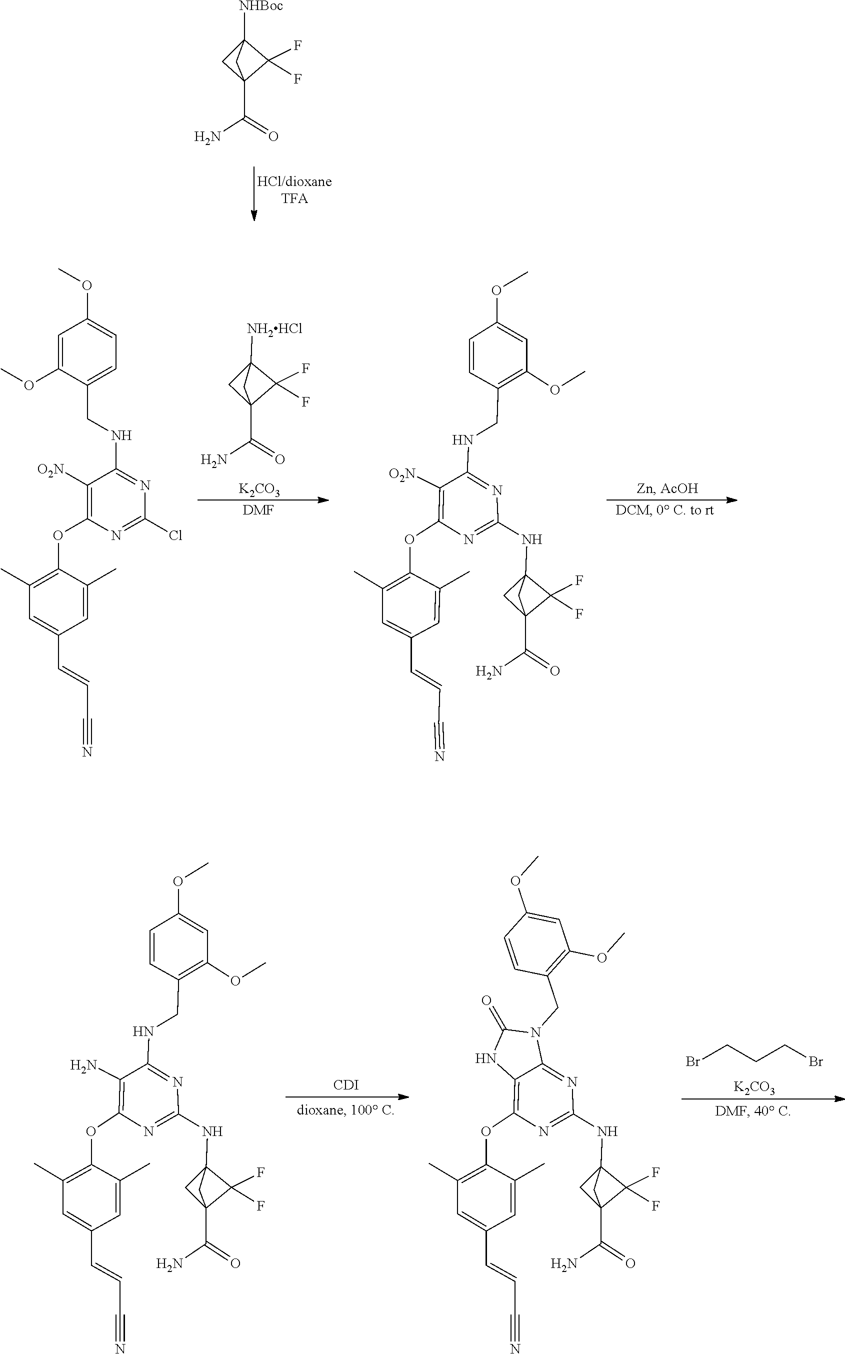

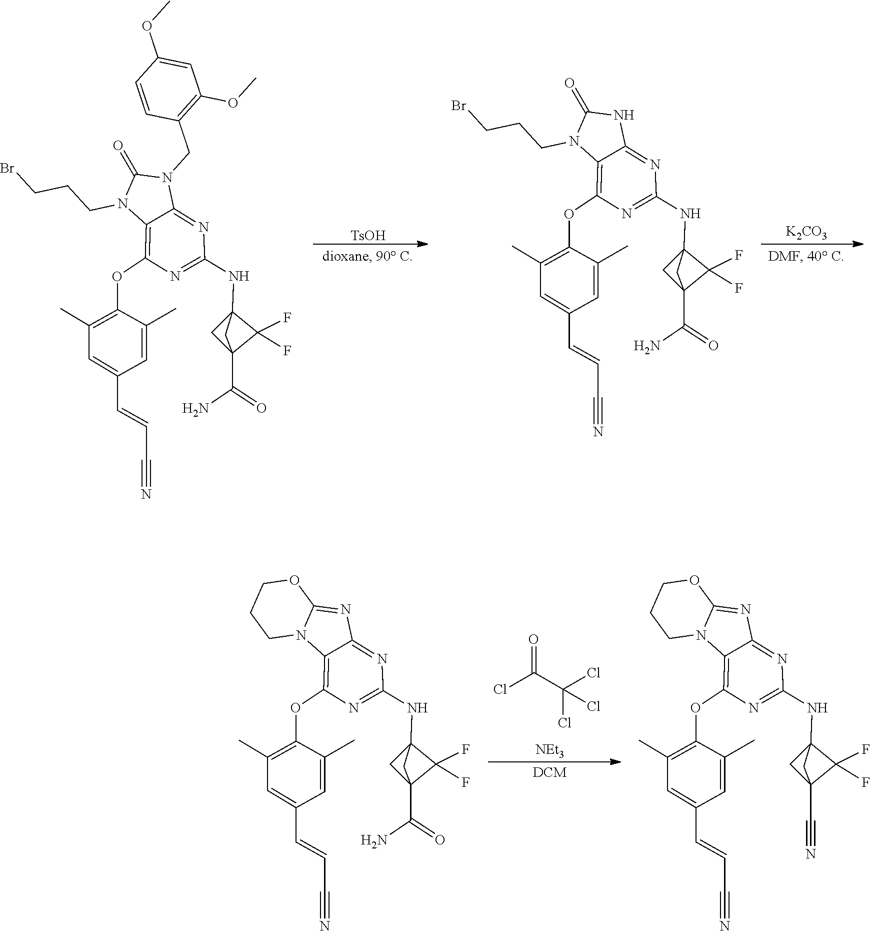

### Step 1a, Preparation of 3-amino-2,2-difluorobicyclo[1.1.1]pentane-1-carboxamide hydrochloride (Intermediate 188a)

To a mixture of tert-butyl N-(3-carbamoyl-2,2-difluoro-1-bicyclo[1.1.1]pentanyl)carbamate (249 mg, 0.949 mmol) in trifluoroacetic acid (8.00 mL) was added hydrochloric acid (4.00 mol/L in dioxane, 2.37 mL, 9.49 mmol), and the mixture was stirred at rt for 1 h. 50 mL of Et₂O was added, followed by 25 mL hexane, and the resulting solid was filtered and washed with 2×10 mL hexane. The solid was dried under vacuum and used without further purification.

### Step 1b, preparation of (E)-3-((4-(4-(2-cyanovinyl)-2,6-dimethylphenoxy)-6-((2,4-dimethoxybenzyl)amino)-5-nitropyrimidin-2-yl)amino)-2,2-difluorobicyclo[1.1.1]pentane-1-carboxamide (Intermediate 188b)

3-amino-2,2-difluoro-bicyclo[1.1.1]pentane-1-carboxamide hydrochloride (48.1 mg, 0.242 mmol), (E)-3-[4-[2-chloro-6-[(2,4-dimethoxyphenyl)methylamino]-5-nitro-pyrimidin-4-yl]oxy-3,5-dimethyl-phenyl]prop-2-enenitrile (100 mg, 0.202 mmol), and potassium carbonate (139 mg, 1.01 mmol) were combined, and DMF (1.00 mL) was added. The mixture was heated to 40° C. for 12. The mixture was diluted with EtOAc, washed twice with brine, and the organics were then dried over MgSO₄, filtered, and concentrated in vacuo. The resulting residue was purified by silica gel chromatography (0-100% EtOAc/hex). MS (m/z) 621.9 [M+H]+.

### Step 2, Preparation of Intermediate 188c

(E)-3-((5-amino-4-(4-(2-cyanovinyl)-2,6-dimethylphenoxy)-6-((2,4-dimethoxybenzyl)amino)pyrimidin-2-yl)amino)-2,2-difluorobicyclo[1.1.1]pentane-1-carboxamide was prepared using the procedure described for the synthesis of (E)-3-(4-((5-amino-2-chloro-6-((2,4-dimethoxybenzyl)amino)pyrimidin-4-yl)oxy)-3,5-dimethylphenyl)acrylonitrile (Intermediate 176b) with the following modifications: (E)-3-((4-(4-(2-cyanovinyl)-2,6-dimethylphenoxy)-6-((2,4-dimethoxybenzyl)amino)-5-nitropyrimidin-2-yl)amino)-2,2-difluorobicyclo[1.1.1]pentane-1-carboxamide was used in place of (E)-3-(4-((2-chloro-6-((2,4-dimethoxybenzyl)amino)-5-nitropyrimidin-4-yl)oxy)-3, 5-dimethylphenyl)acrylonitrile. MS (m/z) 591.9 [M+H]+.

### Step 3, Preparation of Intermediate 188d

(E)-3-((6-(4-(2-cyanovinyl)-2,6-dimethylphenoxy)-9-(2,4-dimethoxybenzyl)-8-oxo-8,9-dihydro-7H-purin-2-yl)amino)-2,2-difluorobicyclo[1.1.1]pentane-1-carboxamide was prepared using the procedure described for the synthesis of (E)-3-((6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile (Intermediate 184a) with the following modifications: (E)-3-((5-amino-4-(4-(2-cyanovinyl)-2,6-dimethylphenoxy)-6-((2,4-dimethoxybenzyl)amino)pyrimidin-2-yl)amino)-2,2-difluorobicyclo[1.1.1]pentane-1-carboxamide was used in place of (E)-3-((5-amino-4-(4-(2-cyanovinyl)-2,6-dimethylphenoxy)-6-((2,4-dimethoxybenzyl)amino)pyrimidin-2-yl)amino)bicyclo[1.1.1]pentane-1-carbonitrile. MS (m/z) 617.9 [M+H]+.

### Step 4, Preparation of Intermediate 188e

(E)-3-((7-(3-bromopropyl)-6-(4-(2-cyanovinyl)-2,6-dimethylphenoxy)-9-(2,4-dimethoxybenzyl)-8-oxo-8,9-dihydro-7H-purin-2-yl)amino)-2,2-difluorobicyclo[1.1.1]pentane-1-carboxamide was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: (E)-3-((6-(4-(2-cyanovinyl)-2,6-dimethylphenoxy)-9-(2,4-dimethoxybenzyl)-8-oxo-8,9-dihydro-7H-purin-2-yl)amino)-2,2-difluorobicyclo[1.1.1]pentane-1-carboxamide and 1,3-dibromopropane were used in place of (E)-3-((6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile and 2-bromoethanol, respectively. MS (m/z) 737.7 [M+H]+.

### Step 5, Preparation of Intermediate 188f

(E)-3-((7-(3-bromopropyl)-6-(4-(2-cyanovinyl)-2,6-dimethylphenoxy)-8-oxo-8,9-dihydro-7H-purin-2-yl)amino)-2,2-difluorobicyclo[1.1.1]pentane-1-carboxamide was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-((7-(3-bromopropyl)-6-(4-(2-cyanovinyl)-2,6-dimethylphenoxy)-9-(2,4-dimethoxybenzyl)-8-oxo-8,9-dihydro-7H-purin-2-yl)amino)-2,2-difluorobicyclo[1.1.1]pentane-1-carboxamide was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 587.7 [M+H]+.

### Step 6, Preparation of Intermediate 188g

(E)-3-((4-(4-(2-cyanovinyl)-2,6-dimethylphenoxy)-7,8-dihydro-6H-[1,3]oxazino[2,3-f]purin-2-yl)amino)-2,2-difluorobicyclo[1.1.1]pentane-1-carboxamide was prepared using the procedure described for the synthesis of (E)-3-((4′-(4-(2-cyanovinyl)-2,6-dimethylphenoxy)-6′H,8′H-spiro[cyclopropane-1,7′-[1,3]oxazino[2,3-f]purin]-2′-yl)amino)bicyclo[1.1.1]pentane-1-carbonitrile (Compound 187) with the following modifications: (E)-3-((7-(3-bromopropyl)-6-(4-(2-cyanovinyl)-2,6-dimethylphenoxy)-8-oxo-8,9-dihydro-7H-purin-2-yl)amino)-2,2-difluorobicyclo[1.1.1]pentane-1-carboxamide was used in place of (E)-3-((7-((1-(bromomethyl)cyclopropyl)methyl)-6-(4-(2-cyanovinyl)-2,6-dimethylphenoxy)-8-oxo-8,9-dihydro-7H-purin-2-yl)amino)bicyclo[1.1.1]pentane-1-carbonitrile. MS (m/z) 507.9 [M+H]+.

### Step 7, preparation of (E)-3-((4-(4-(2-cyanovinyl)-2,6-dimethylphenoxy)-7,8-dihydro-6H-[1,3]oxazino[2,3-f]purin-2-yl)amino)-2,2-difluorobicyclo[1.1.1]pentane-1-carbonitrile (Compound 188)

To a mixture of (E)-3-((4-(4-(2-cyanovinyl)-2,6-dimethylphenoxy)-7,8-dihydro-6H-[1,3]oxazino[2,3-f]purin-2-yl)amino)-2,2-difluorobicyclo[1.1.1]pentane-1-carboxamide (15.6 mg, 0.0307 mmol) in dichloromethane (0.500 mL) was added triethylamine (0.0257 mL, 0.184 mmol) and trichloroacetyl chloride (16.8 mg, 0.0922 mmol). The mixture was stirred at rt for 30 min, then basified with sat. NaHCO₃ solution, diluted with DMF/H₂O and purified by preparative HPLC (0-100% MeCN/water) to afford the title compound. MS (m/z) 489.9 [M+H]+. ¹H NMR (400 MHz, Acetonitrile-d3) δ 7.48 (d, J=16.7 Hz, 1H), 7.37 (s, 2H), 6.19 (s, 1H), 6.06 (d, J=16.7 Hz, 1H), 4.58-4.48 (m, 2H), 4.34 (t, J=6.1 Hz, 2H), 2.34-2.26 (m, 2H), 2.26-2.22 (m, 2H), 2.13 (s, 10H). ¹⁹F NMR (376 MHz, Acetonitrile-d3) δ −121.00 (t, J=10.1 Hz).

### Example 189

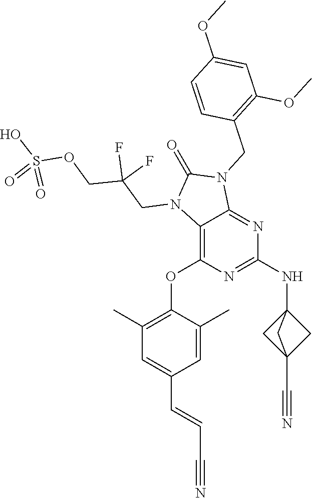 [SMILES: COc1ccc(Cn2c(=O)n(CC(F)(F)COS(=O)(=O)O)c3c(Oc4c(C)cc(/C=C/C#N)cc4C)nc(NC45CC(C#N)(C4)C5)nc32)c(OC)c1]

(E)-3-(2-((3-cyanobicyclo[1.1.1]pentan-1-yl)amino)-6-(4-(2-cyanovinyl)-2,6-dimethylphenoxy)-9-(2,4-dimethoxybenzyl)-8-oxo-8,9-dihydro-7H-purin-7-yl)-2,2-difluoropropyl hydrogen sulfate (Intermediate 189a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: 5,5-difluoro-1,3,2-dioxathiane 2,2-dioxide was used in place of 2-bromoethanol. MS (m/z) 737.9 [M+H]+.

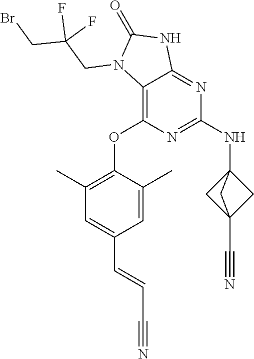 [SMILES: Cc1cc(/C=C/C#N)cc(C)c1Oc1nc(NC23CC(C#N)(C2)C3)nc2[nH]c(=O)n(CC(F)(F)CBr)c12]

(E)-3-((6-(4-(2-cyanovinyl)-2,6-dimethylphenoxy)-7-(2,2-difluoro-3-hydroxypropyl)-8-oxo-8,9-dihydro-7H-purin-2-yl)amino)bicyclo[1.1.1]pentane-1-carbonitrile (Intermediate 189b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-(2-((3-cyanobicyclo[1.1.1]pentan-1-yl)amino)-6-(4-(2-cyanovinyl)-2,6-dimethylphenoxy)-9-(2,4-dimethoxybenzyl)-8-oxo-8,9-dihydro-7H-purin-7-yl)-2,2-difluoropropyl hydrogen sulfate was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 508.0 [M+H]+.

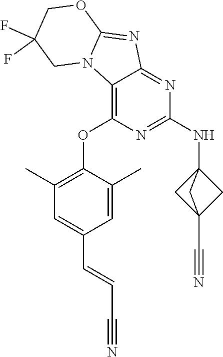 [SMILES: Cc1cc(/C=C/C#N)cc(C)c1Oc1nc(NC23CC(C#N)(C2)C3)nc2nc3n(c12)CC(F)(F)CO3]

(E)-3-((4-(4-(2-cyanovinyl)-2,6-dimethylphenoxy)-7,7-difluoro-7,8-dihydro-6H-[1,3]oxazino[2,3-f]purin-2-yl)amino)bicyclo[1.1.1]pentane-1-carbonitrile (Compound 189) was prepared using the procedure described for the synthesis of (E)-3-((4-(4-(2-cyanovinyl)-2,6-dimethylphenoxy)-6,7-dihydrooxazolo[2,3-f]purin-2-yl)amino)bicyclo[1.1.1]pentane-1-carbonitrile (Compound 184) with the following modifications: (E)-3-((6-(4-(2-cyanovinyl)-2,6-dimethylphenoxy)-7-(2,2-difluoro-3-hydroxypropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile. MS (m/z) 490.0 [M+H]+. ¹H NMR (400 MHz, DMSO-d6) δ 7.73 (s, 1H), 7.64 (d, J=16.7 Hz, 1H), 7.55 (s, 2H), 6.44 (d, J=16.7 Hz, 1H), 4.98-4.82 (m, 4H), 2.20-1.97 (m, 12H). ¹⁹F NMR (376 MHz, DMSO-d6) δ −110.84 (p, J=11.9 Hz).

### Example 190

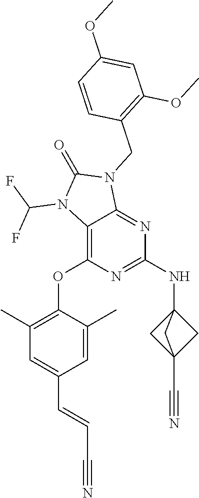

(E)-3-((6-(4-(2-cyanovinyl)-2,6-dimethylphenoxy)-7-(difluoromethyl)-9-(2,4-dimethoxybenzyl)-8-oxo-8,9-dihydro-7H-purin-2-yl)amino)bicyclo[1.1.1]pentane-1-carbonitrile (Intermediate 190a) was prepared as follows: To a mixture of (E)-3-((6-(4-(2-cyanovinyl)-2,6-dimethylphenoxy)-9-(2,4-dimethoxybenzyl)-8-oxo-8,9-dihydro-7H-purin-2-yl)amino)bicyclo[1.1.1]pentane-1-carbonitrile (25.0 mg, 0.0444 mmol) and [chloro(difluoro)methyl]sulfonylbenzene (0.0201 g, 0.0887 mmol) in MeCN (0.350 mL) was added potassium hydroxide (4.50 mol/L, 0.108 mL, 0.488 mmol). The mixture was heated to 50° C. for 3 h, then cooled to rt, diluted with water and extracted with EtOAc. Combined organics were dried over MgSO₄, filtered, and concentrated in vacuo. The resulting residue was purified by silica gel chromatography (0-100% EtOAc/DCM) to afford the title compound. MS (m/z) 614.1 [M+H]+.

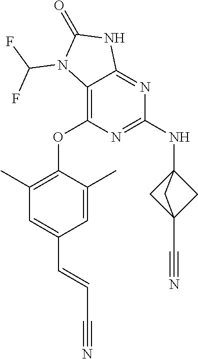 [SMILES: Cc1cc(/C=C/C#N)cc(C)c1Oc1nc(NC23CC(C#N)(C2)C3)nc2[nH]c(=O)n(C(F)F)c12]

(E)-3-((6-(4-(2-cyanovinyl)-2,6-dimethylphenoxy)-7-(difluoromethyl)-8-oxo-8,9-dihydro-7H-purin-2-yl)amino)bicyclo[1.1.1]pentane-1-carbonitrile (Compound 190) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-((6-(4-(2-cyanovinyl)-2,6-dimethylphenoxy)-7-(difluoromethyl)-9-(2,4-dimethoxybenzyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 464.0 [M+H]+. ¹H NMR (400 MHz, DMSO-d6) δ 13.20 (s, 1H), 7.99-7.58 (m, 3H), 7.55 (s, 2H), 6.43 (d, J=16.6 Hz, 1H), 2.20-1.89 (m, 12H). ¹⁹F NMR (376 MHz, DMSO-d6) δ −74.57, −87.20 (d, J=71.4 Hz).

### Examples 191 and 192

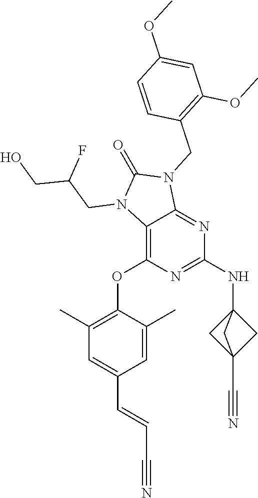 [SMILES: COc1ccc(Cn2c(=O)n(CC(F)CO)c3c(Oc4c(C)cc(/C=C/C#N)cc4C)nc(NC45CC(C#N)(C4)C5)nc32)c(OC)c1]

(E)-3-((6-(4-(2-cyanovinyl)-2,6-dimethylphenoxy)-9-(2,4-dimethoxybenzyl)-7-(2-fluoro-3-hydroxypropyl)-8-oxo-8,9-dihydro-7H-purin-2-yl)amino)bicyclo[1.1.1]pentane-1-carbonitrile (Intermediate 191a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: 3-bromo-2-fluoro-propan-1-ol was used in place of 2-bromoethanol. MS (m/z) 640.0 [M+H]+.

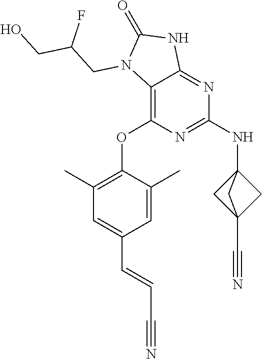 [SMILES: Cc1cc(/C=C/C#N)cc(C)c1Oc1nc(NC23CC(C#N)(C2)C3)nc2[nH]c(=O)n(CC(F)CO)c12]

(E)-3-((6-(4-(2-cyanovinyl)-2,6-dimethylphenoxy)-7-(2-fluoro-3-hydroxypropyl)-8-oxo-8,9-dihydro-7H-purin-2-yl)amino)bicyclo[1.1.1]pentane-1-carbonitrile (Intermediate 191b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-((6-(4-(2-cyanovinyl)-2,6-dimethylphenoxy)-9-(2,4-dimethoxybenzyl)-7-(2-fluoro-3-hydroxypropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 490.0 [M+H]+.

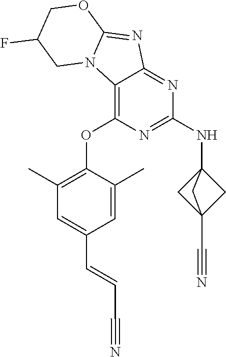 [SMILES: Cc1cc(/C=C/C#N)cc(C)c1Oc1nc(NC23CC(C#N)(C2)C3)nc2nc3n(c12)CC(F)CO3]

(E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Intermediate 191c) was prepared using the procedure described for the synthesis of (E)-3-((4-(4-(2-cyanovinyl)-2,6-dimethylphenoxy)-6,7-dihydrooxazolo[2,3-f]purin-2-yl)amino)bicyclo[1.1.1]pentane-1-carbonitrile (Compound 184) with the following modifications: (E)-3-((6-(4-(2-cyanovinyl)-2,6-dimethylphenoxy)-7-(2-fluoro-3-hydroxypropyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile. MS (m/z) 472.0 [M+H]+.

Preparative chiral SFC (AD-H 4.6×100 mm, 5 mic column, 35% EtOH in CO₂) on (E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Intermediate 191c) afforded the title compounds, (R,E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Compound 191) as the first enantiomer to elute (Peak 1), and (S,E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Compound 192) as the second enantiomer to elute (Peak 2). The absolute configuration was not assigned for Compound 191 and Compound 192.

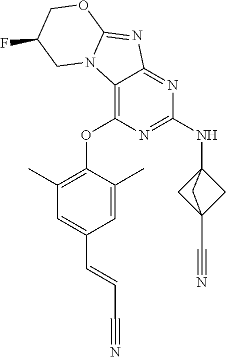 [SMILES: Cc1cc(/C=C/C#N)cc(C)c1Oc1nc(NC23CC(C#N)(C2)C3)nc2nc3n(c12)C[C@@H](F)CO3]

### (R,E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Compound 191, Peak 1)

MS (m/z) 472.0 [M+H]+. ¹H NMR (400 MHz, DMSO-d6) δ 7.68-7.59 (m, 2H), 7.55 (d, J=4.3 Hz, 2H), 6.44 (d, J=16.7 Hz, 1H), 5.55 (d, J=45.4 Hz, 1H), 4.88-4.51 (m, 4H), 2.14 (s, 3H), 2.11 (s, 3H), 2.06 (s, 6H).

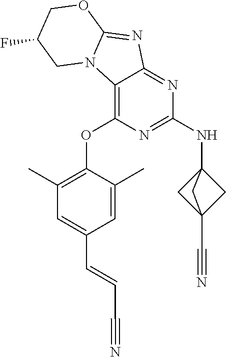 [SMILES: Cc1cc(/C=C/C#N)cc(C)c1Oc1nc(NC23CC(C#N)(C2)C3)nc2nc3n(c12)C[C@H](F)CO3]

### (S,E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Compound 192, Peak 2)

MS (m/z) 472.0 [M+H]+. ¹H NMR (400 MHz, DMSO-d6) δ 7.68-7.58 (m, 2H), 7.55 (d, J=4.5 Hz, 2H), 6.44 (d, J=16.7 Hz, 1H), 5.55 (d, J=45.0 Hz, 1H), 4.86-4.51 (m, 4H), 2.14 (s, 3H), 2.11 (s, 3H), 2.07 (d, J=9.5 Hz, 6H).

### Examples 193 and 194

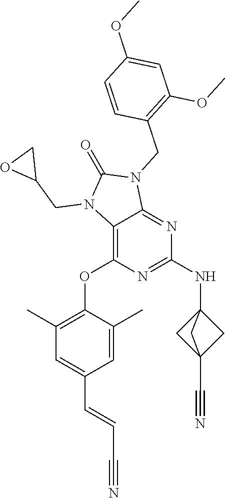 [SMILES: COc1ccc(Cn2c(=O)n(CC3CO3)c3c(Oc4c(C)cc(/C=C/C#N)cc4C)nc(NC45CC(C#N)(C4)C5)nc32)c(OC)c1]

(E)-3-((6-(4-(2-cyanovinyl)-2,6-dimethylphenoxy)-9-(2,4-dimethoxybenzyl)-7-(oxiran-2-ylmethyl)-8-oxo-8,9-dihydro-7H-purin-2-yl)amino)bicyclo[1.1.1]pentane-1-carbonitrile (Intermediate 193a) was prepared using the procedure described for the synthesis of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile (Intermediate 184b) with the following modifications: 1,3-dibromopropan-2-ol was used in place of 2-bromoethanol. MS (m/z) 620.0 [M+H]+.

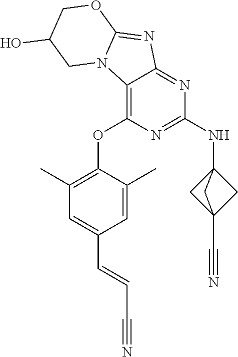 [SMILES: Cc1cc(/C=C/C#N)cc(C)c1Oc1nc(NC23CC(C#N)(C2)C3)nc2nc3n(c12)CC(O)CO3]

(E)-3-((4-(4-(2-cyanovinyl)-2,6-dimethylphenoxy)-7-hydroxy-7,8-dihydro-6H-[1,3]oxazino[2,3-f]purin-2-yl)amino)bicyclo[1.1.1]pentane-1-carbonitrile (Intermediate 193b) was prepared using the procedure described for the synthesis of (E)-3-((6-(4-(2-cyanovinyl)-2,6-dimethylphenoxy)-7-(2-hydroxyethyl)-8-oxo-8,9-dihydro-7H-purin-2-yl)amino)bicyclo[1.1.1]pentane-1-carbonitrile (Intermediate 184c) with the following modifications: (E)-3-((6-(4-(2-cyanovinyl)-2,6-dimethylphenoxy)-9-(2,4-dimethoxybenzyl)-7-(oxiran-2-ylmethyl)-8-oxo-8,9-dihydro-7H-purin-2-yl)amino)bicyclo[1.1.1]pentane-1-carbonitrile was used in place of (E)-3-((6-(4-(2-cyanovinyl)-2,6-dimethylphenoxy)-9-(2,4-dimethoxybenzyl)-7-(2-hydroxyethyl)-8-oxo-8,9-dihydro-7H-purin-2-yl)amino)bicyclo[1.1.1]pentane-1-carbonitrile. MS (m/z) 470.0 [M+H]+.

Preparative chiral SFC (1K −5 um-4.5×100 mm column, 45% EtOH in CO₂) on (E)-3-((4-(4-(2-cyanovinyl)-2,6-dimethylphenoxy)-7-hydroxy-7,8-dihydro-6H-[1,3]oxazino[2,3-f]purin-2-yl)amino)bicyclo[1.1.1]pentane-1-carbonitrile (Intermediate 193b) afforded the title compounds, (R,E)-3-((4-(4-(2-cyanovinyl)-2,6-dimethylphenoxy)-7-hydroxy-7,8-dihydro-6H-[1,3]oxazino[2,3-f]purin-2-yl)amino)bicyclo[1.1.1]pentane-1-carbonitrile (Compound 193) as the first enantiomer to elute (Peak 1), and (S,E)-3-((4-(4-(2-cyanovinyl)-2,6-dimethylphenoxy)-7-hydroxy-7,8-dihydro-6H-[1,3]oxazino[2,3-f]purin-2-yl)amino)bicyclo[1.1.1]pentane-1-carbonitrile (Compound 194) as the second enantiomer to elute (Peak 2). The absolute configuration was not assigned for Compound 193 and Compound 194.

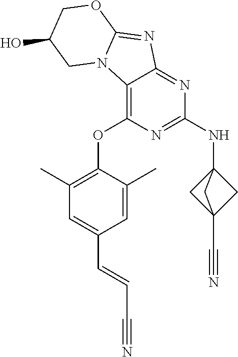 [SMILES: Cc1cc(/C=C/C#N)cc(C)c1Oc1nc(NC23CC(C#N)(C2)C3)nc2nc3n(c12)C[C@@H](O)CO3]

### (R,E)-3-((4-(4-(2-cyanovinyl)-2,6-dimethylphenoxy)-7-hydroxy-7,8-dihydro-6H-[1,3]oxazino[2,3-f]purin-2-yl)amino)bicyclo[1.1.1]pentane-1-carbonitrile (Compound 193, Peak 1)

MS (m/z) 470.0 [M+H]+. ¹H NMR (400 MHz, DMSO-d6) δ 7.62 (d, J=16.7 Hz, 1H), 7.54 (s, 3H), 6.43 (d, J=16.7 Hz, 1H), 5.72 (d, J=3.4 Hz, 1H), 4.54 (d, J=11.2 Hz, 1H), 4.48-4.38 (m, 2H), 4.38-4.30 (m, 1H), 4.20 (d, J=12.4 Hz, 1H), 2.16-1.96 (m, 12H).

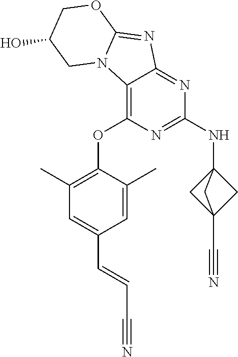 [SMILES: Cc1cc(/C=C/C#N)cc(C)c1Oc1nc(NC23CC(C#N)(C2)C3)nc2nc3n(c12)C[C@H](O)CO3]

### (S,E)-3-((4-(4-(2-cyanovinyl)-2,6-dimethylphenoxy)-7-fluoro-7,8-dihydro-6H-[1,3]oxazino[2,3-f]purin-2-yl)amino)bicyclo[1.1.1]pentane-1-carbonitrile (Compound 194, Peak 2)

MS (m/z) 470.0 [M+H]+. ¹H NMR (400 MHz, DMSO-d6) δ 7.62 (d, J=16.6 Hz, 1H), 7.54 (s, 3H), 6.43 (d, J=16.7 Hz, 1H), 5.72 (s, 1H), 4.54 (d, J=11.1 Hz, 1H), 4.48-4.38 (m, 2H), 4.38-4.31 (m, 1H), 4.20 (d, J=12.4 Hz, 1H), 2.20-1.90 (m, 12H).

### Example 195

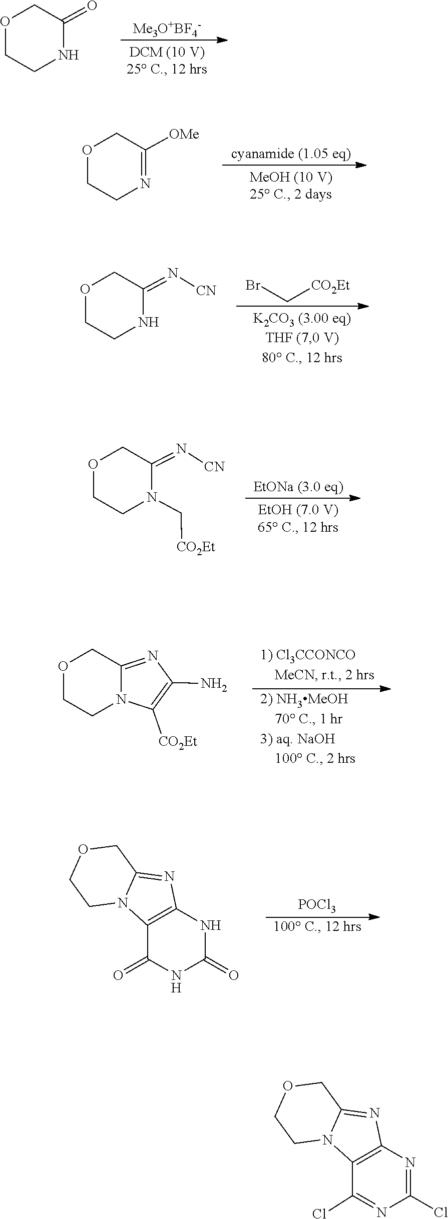

### Step 1, Preparation of 5-methoxy-3,6-dihydro-2H-1,4-oxazine (Intermediate 195a)

DCM (800 mL) was charged into a reactor at 10-20° C. Morpholin-3-one (80.0 g, 0.79 mol) was charged into the reactor at 10-20° C. under N₂. Me₃OBF₄ (163 g, 1.10 mol) was charged into the reactor at 10-20° C., and the mixture was then stirred at 10-20° C. for 12 h. The mixture was adjusted to pH=8-9 with saturated Na₂CO₃ solution, then extracted with DCM (500 mL). The organic phase was washed with brine, dried over Na₂SO₄, and concentrated in vacuo to obtain crude product which was used directly.

### Step 2, Preparation of (Z)—N-(morpholin-3-ylidene)cyanamide (Intermediate 195b)

MeOH (180 mL) was charged into a reactor at 10-20° C. 5-methoxy-3,6-dihydro-2H-1,4-oxazine (18.0 g, 0.15 mol) was charged into the reactor at 10-20° C. under N₂. Cyanamide (6.8 g, 0.16 mol) was charged into the reactor at 10-20° C., and the mixture was then stirred at 10-20° C. for 12 h. The mixture was filtered and the filter cake was washed twice with MeOH (9.00 mL). The filter cake was dried to give the crude product which was used directly

### Step 3, preparation of ethyl (Z)-2-(3-(cyanoimino)morpholino)acetate (Intermediate 195c)

THE (105 mL) was charged into a reactor at 10-20° C. (Z)—N-(morpholin-3-ylidene)cyanamide (15.0 g, 0.12 mol) was charged into the reactor at 10-20° C. under N₂. Ethyl 2-bromoacetate (21.5 g, 0.13 mol) was charged into the reactor at 10-20° C., followed by K₂CO₃ (44.5 g, 0.35 mol), and the mixture was then stirred at 80° C. for 2 h. H₂O (75 mL) and ethyl acetate (75 mL) were charged into the mixture. The organic phase was separated and washed with brine, dried over Na₂SO₄, filtered, and concentrated in vacuo. The crude product was purified by silica gel flash column chromatography (1-100% EtOAc/petroleum ether) to afford the title compound.

### Step 4, preparation of ethyl 2-amino-5,6-dihydro-8H-imidazo[2,1-c][1,4]oxazine-3-carboxylate (Intermediate 195d)

EtOH (91 mL) was charged into a reactor at 10-20° C. Ethyl (Z)-2-(3-(cyanoimino)morpholino)acetate (13.0 g, 0.062 mol) was charged into the reactor at 10-20° C. under N₂. EtONa (13.0 g, 0.191 mol) was charged into the reactor at 10-20° C., and the mixture was then stirred at 65° C. for 2 h. H₂O (130 mL) and ethyl acetate (130 mL) were charged into the mixture. The organic phase was separated and washed with brine, dried over Na₂SO₄, filtered, and concentrated in vacuo to give the crude product.

### Step 5, Preparation of 6,7-dihydro-1H-[1,4]oxazino[3,4-f]purine-2,4(3H,9H)-dione (Intermediate 195e)

MeCN (60.0 mL) was charged into a reactor at 10-20° C. Ethyl 2-amino-5,6-dihydro-8H-imidazo[2,1-c][1,4]oxazine-3-carboxylate (10.0 g, 0.047 mol) was charged into the reactor at 10-20° C. under N₂. Trichloroacetyl isocyanate (11.5 g, 0.061 mol) was charged into the reactor at 10-20° C., and the mixture was then stirred at 15-25° C. for 2 h. The mixture was concentrated below 45° C. in vacuo. MeOH (20.0 mL) was charged into the reactor, followed by NH₃ (100 mL, 7M in MeOH), and the mixture stirred at 60° C. for 30 min. The mixture was concentrated below 45° C. in vacuo. MeOH (20.0 mL) was charged into the reactor, followed by NaOH (70.0 mL, 1M in H₂O), and the mixture stirred at 100° C. for 2 h, then at 10° C. for 2 h. The mixture was filtered and the filter cake was dried to give the crude product. MS (m/z) 209.2 [M+H]+.

### Step 6, Preparation of 2,4-dichloro-6,7-dihydro-9H-[1,4]oxazino[3,4-f]purine (Intermediate 195f)

POCl₃ (80 mL) was charged into a reactor at 10-20° C. 6,7-dihydro-1H-[1,4]oxazino[3,4-f]purine-2,4(3H,9H)-dione (8.00 g, 0.038 mol) was charged into the reactor at 10-20° C. under N₂. The mixture was then stirred at 100° C. for 12 h. The mixture was concentrated below 45° C. in vacuo. Ethyl acetate (80.0 mL) and 10% Na₂CO₃ (80.0 mL) were charged into the mixture. The organic phase was separated and washed with brine, dried over Na₂SO₄, filtered, and concentrated in vacuo. The crude product was purified by silica gel flash column chromatography (1-100% EtOAc/petroleum ether) to afford the title compound. MS (m/z) 244.9 [M+H]+. ¹H NMR (400 MHz, DMSO-d6) δ 5.08 (s, 1H) 4.48 (t, J=5.19 Hz, 1H) 4.17 (t, J=5.19 Hz, 1H).

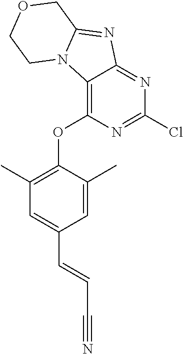 [SMILES: Cc1cc(/C=C/C#N)cc(C)c1Oc1nc(Cl)nc2nc3n(c12)CCOC3]

(E)-3-(4-((2-chloro-6,7-dihydro-9H-[1,4]oxazino[3,4-f]purin-4-yl)oxy)-3,5-dimethylphenyl)acrylonitrile (Intermediate 195g) was prepared using the procedure described for (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,4-dichloro-6,7-dihydro-9H-[1,4]oxazino[3,4-f]purine was used in place of 2,6-dichloro-N-(2,4-dimethoxybenzyl)-5-nitropyrimidin-4-amine. MS (m/z) 382.0 [M+H]+.

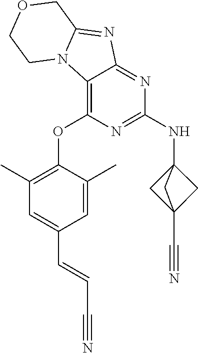 [SMILES: Cc1cc(/C=C/C#N)cc(C)c1Oc1nc(NC23CC(C#N)(C2)C3)nc2nc3n(c12)CCOC3]

(E)-3-((4-(4-(2-cyanovinyl)-2,6-dimethylphenoxy)-6,7-dihydro-9H-[1,4]oxazino[3,4-f]purin-2-yl)amino)bicyclo[1.1.1]pentane-1-carbonitrile (Compound 195) was prepared using the procedure described for the synthesis of (E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile (Compound 13) with the following modifications: (E)-3-(4-((2-chloro-6,7-dihydro-9H-[1,4]oxazino[3,4-f]purin-4-yl)oxy)-3,5-dimethylphenyl)acrylonitrile was used in place of (E)-3-(4-((2-chloro-7-(difluoromethyl)-8-methyl-7H-purin-6-yl)oxy)-3,5-dimethylphenyl)acrylonitrile. MS (m/z) 454.1 [M+H]+. ¹H NMR (400 MHz, Acetonitrile-d3) δ 7.49 (d, J=16.7 Hz, 1H), 7.39 (s, 2H), 6.07 (d, J=16.7 Hz, 1H), 5.97 (s, 1H), 4.91 (s, 2H), 4.43-4.37 (m, 2H), 4.19-4.12 (m, 2H), 2.13-2.09 (m, 12H).

### Example 196

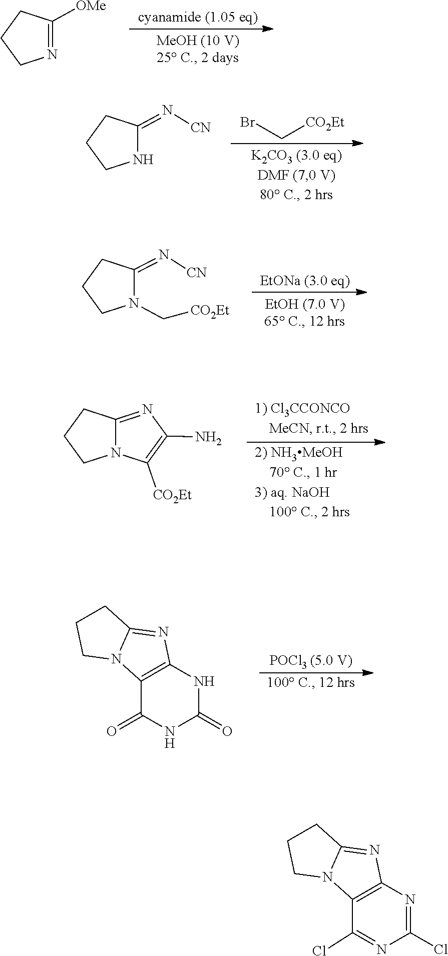

(Z)—N-(pyrrolidin-2-ylidene)cyanamide (Intermediate 196a) was prepared using the procedure described for the synthesis of (Z)—N-(morpholin-3-ylidene)cyanamide (Intermediate 195b) with the following modifications: 5-methoxy-3,4-dihydro-2H-pyrrole was used in place of 5-methoxy-3,6-dihydro-2H-1,4-oxazine.

ethyl (Z)-2-(2-(cyanoimino)pyrrolidin-1-yl)acetate (Intermediate 196b) was prepared using the procedure described for the synthesis of ethyl (Z)-2-(3-(cyanoimino)morpholino)acetate (Intermediate 195c) with the following modifications: (Z)—N-(pyrrolidin-2-ylidene)cyanamide was used in place of (Z)—N-(morpholin-3-ylidene)cyanamide.

Ethyl 2-amino-6,7-dihydro-5H-pyrrolo[1,2-a]imidazole-3-carboxylate (Intermediate 196c) was prepared using the procedure described for the synthesis of ethyl 2-amino-5,6-dihydro-8H-imidazo[2,1-c][1,4]oxazine-3-carboxylate (Intermediate 195d) with the following modifications: ethyl (Z)-2-(2-(cyanoimino)pyrrolidin-1-yl)acetate was used in place of ethyl (Z)-2-(3-(cyanoimino)morpholino)acetate.

7,8-dihydro-1H-pyrrolo[2,1-f]purine-2,4(3H,6H)-dione (Intermediate 196d) was prepared using the procedure described for the synthesis of 6,7-dihydro-1H-[1,4]oxazino[3,4-f]purine-2,4(3H,9H)-dione (Intermediate 195e) with the following modifications: ethyl 2-amino-6,7-dihydro-5H-pyrrolo[1,2-a]imidazole-3-carboxylate was used in place of ethyl 2-amino-5,6-dihydro-8H-imidazo[2,1-c][1,4]oxazine-3-carboxylate. MS (m/z) 193.2 [M+H]+.

2,4-dichloro-7,8-dihydro-6H-pyrrolo[2,1-f]purine (Intermediate 196e) was prepared using the procedure described for the synthesis of 2,4-dichloro-6,7-dihydro-911-[1,4]oxazino[3,4-f]purine (Intermediate 195f) with the following modifications: 7,8-dihydro-1H-pyrrolo[2,1-f]purine-2,4(3H,6H)-dione was used in place of 6,7-dihydro-1H-[1,4]oxazino[3,4-f]purine-2,4(3H,9H)-dione. MS (m/z) 229.0 [M+H]+. ¹H NMR (400 MHz, DMSO-d6) δ 4.37-4.49 (m, 1H) 4.42 (t, J=7.19 Hz, 1H) 3.19 (t, J=7.69 Hz, 1H) 3.14-3.26 (m, 1H) 2.72 (q, J=7.44 Hz, 2H).

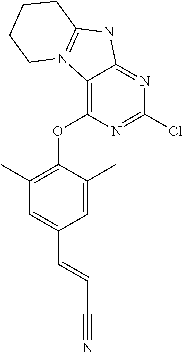

(E)-3-(4-((2-chloro-7,8-dihydro-6H-pyrrolo[2,1-f]purin-4-yl)oxy)-3,5-dimethylphenyl)acrylonitrile (Intermediate 196f) was prepared using the procedure described for (E)-3-(4-((2-chloro-6-((2,4-dimethoxybenzyl)amino)-5-nitropyrimidin-4-yl)oxy)-3,5-dimethylphenyl)acrylonitrile (Intermediate 176a) with the following modifications: 2,4-dichloro-7,8-dihydro-6H-pyrrolo[2,1-f]purine was used in place of 2,6-dichloro-N-(2,4-dimethoxybenzyl)-5-nitropyrimidin-4-amine. MS (m/z) 366.0 [M+H]+.

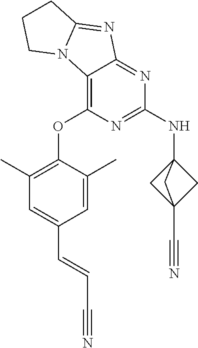 [SMILES: Cc1cc(/C=C/C#N)cc(C)c1Oc1nc(NC23CC(C#N)(C2)C3)nc2nc3n(c12)CCC3]

(E)-3-((4-(4-(2-cyanovinyl)-2,6-dimethylphenoxy)-7,8-dihydro-6H-pyrrolo[2,1-f]purin-2-yl)amino)bicyclo[1.1.1]pentane-1-carbonitrile (Compound 196) was prepared using the procedure described for the synthesis of (E)-3-((6-(4-(2-cyanovinyl)-2,6-dimethylphenoxy)-7-(difluoromethyl)-8-methyl-7H-purin-2-yl)amino)bicyclo[1.1.1]pentane-1-carbonitrile (Compound 13) with the following modifications: (E)-3-(4-((2-chloro-7,8-dihydro-6H-pyrrolo[2,1-f]purin-4-yl)oxy)-3,5-dimethylphenyl)acrylonitrile was used in place of (E)-3-(4-((2-chloro-7-(difluoromethyl)-8-methyl-7H-purin-6-yl)oxy)-3,5-dimethylphenyl)acrylonitrile. MS (m/z) 438.0 [M+H]+. ¹H NMR (400 MHz, DMSO-d6) δ 7.68-7.57 (m, 2H), 7.54 (s, 2H), 6.43 (d, J=16.7 Hz, 1H), 4.28 (t, J=7.1 Hz, 2H), 2.98 (t, J=7.6 Hz, 2H), 2.66-2.57 (m, 2H), 2.20-1.91 (m, 12H).

### Example 197

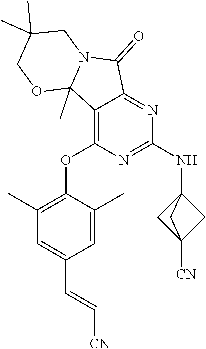 [SMILES: Cc1cc(/C=C/C#N)cc(C)c1Oc1nc(NC23CC(C#N)(C2)C3)nc2c1C1(C)OCC(C)(C)CN1C2=O]

(E)-3-((4-(4-(2-cyanovinyl)-2,6-dimethylphenoxy)-4b,7,7-trimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: 3-amino-2,2-dimethyl-propan-1-ol was used in place of (R)-3-amino-2-methoxypropan-1-ol. MS (m/z) 511.0 [M+H]+. ¹H NMR (400 MHz, DMSO-d6) δ 8.75 (s, 1H), 7.63 (d, J=16.6 Hz, 1H), 7.57 (s, 2H), 6.44 (d, J=16.7 Hz, 1H), 3.86 (dd, J=23.1, 12.4 Hz, 2H), 3.41 (s, 1H), 3.18 (d, J=13.2 Hz, 1H), 2.15-2.02 (m, 6H), 2.02-1.91 (m, 6H), 1.88 (s, 3H), 0.93 (s, 3H), 0.83 (s, 3H).

### Example 198

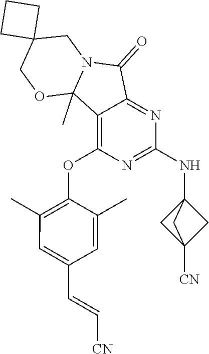 [SMILES: Cc1cc(/C=C/C#N)cc(C)c1Oc1nc(NC23CC(C#N)(C2)C3)nc2c1C1(C)OCC3(CCC3)CN1C2=O]

(E)-3-((4′-(4-(2-cyanovinyl)-2,6-dimethylphenoxy)-4b′-methyl-10′-oxo-4b′,10′-dihydro-6′H,8′H-spiro[cyclobutane-1,7′-pyrimido[5′,4′:3,4]pyrrolo[2,1-b][1,3]oxazin]-2′-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: [1-(aminomethyl)cyclobutyl]methanol was used in place of (R)-3-amino-2-methoxypropan-1-ol and the mixture was heated to 140° C. for 2 h. MS (m/z) 522.9 [M+H]+. ¹H NMR (400 MHz, DMSO-d6) δ 8.74 (s, 1H), 7.63 (d, J=16.6 Hz, 1H), 7.57 (d, J=6.5 Hz, 2H), 6.44 (d, J=16.6 Hz, 1H), 4.31-4.19 (m, 1H), 3.96 (d, J=11.8 Hz, 1H), 3.78 (d, J=11.7 Hz, 1H), 3.30-3.23 (m, 1H), 2.08 (d, J=10.3 Hz, 6H), 2.02-1.91 (m, 6H), 1.91-1.83 (m, 4H), 1.83-1.73 (m, 3H), 1.73-1.60 (m, 2H).

### Example 199

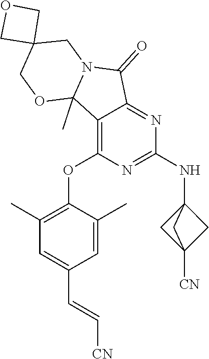 [SMILES: Cc1cc(/C=C/C#N)cc(C)c1Oc1nc(NC23CC(C#N)(C2)C3)nc2c1C1(C)OCC3(COC3)CN1C2=O]

(E)-3-((4′-(4-(2-cyanovinyl)-2,6-dimethylphenoxy)-4b′-methyl-10′-oxo-4b′,10′-dihydro-6′H,8′H-spiro[oxetane-3,7′-pyrimido[5′,4′:3,4]pyrrolo[2,1-b][1,3]oxazin]-2′-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: [3-(aminomethyl)oxetan-3-yl]methanol was used in place of (R)-3-amino-2-methoxypropan-1-ol. MS (m/z) 524.9 [M+H]+. ¹H NMR (400 MHz, DMSO-d6) δ 8.78 (s, 1H), 7.63 (d, J=16.6 Hz, 1H), 7.57 (d, J=7.1 Hz, 2H), 6.44 (d, J=16.6 Hz, 1H), 4.59 (d, J=13.4 Hz, 1H), 4.35 (dd, J=10.8, 6.3 Hz, 2H), 4.29-4.11 (m, 4H), 3.55 (d, J=13.4 Hz, 1H), 2.08 (d, J=11.4 Hz, 6H), 2.01-1.84 (m, 9H).

### Example 200

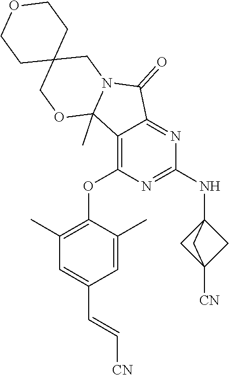 [SMILES: Cc1cc(/C=C/C#N)cc(C)c1Oc1nc(NC23CC(C#N)(C2)C3)nc2c1C1(C)OCC3(CCOCC3)CN1C2=O]

(E)-3-((4′-(4-(2-cyanovinyl)-2,6-dimethylphenoxy)-4b′-methyl-10′-oxo-2,3,4b′,5,6,10′-hexahydro-6′H,8′H-spiro[pyran-4,7′-pyrimido[5′,4′:3,4]pyrrolo[2,1-b][1,3]oxazin]-2′-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: [4-(aminomethyl)tetrahydropyran-4-yl]methanol was used in place of (R)-3-amino-2-methoxypropan-1-ol. MS (m/z) 552.9 [M+H]+. ¹H NMR (400 MHz, DMSO-d6) δ 8.76 (s, 1H), 7.63 (d, J=16.7 Hz, 1H), 7.57 (s, 2H), 6.44 (d, J=16.6 Hz, 1H), 4.30 (d, J=13.5 Hz, 1H), 3.89 (d, J=12.1 Hz, 1H), 3.70 (d, J=12.0 Hz, 1H), 3.56 (q, J=8.1, 6.8 Hz, 4H), 3.18 (d, J=13.5 Hz, 1H), 2.08 (d, J=13.5 Hz, 6H), 2.02-1.83 (m, 9H), 1.52-1.32 (m, 3H), 1.27 (d, J=13.9 Hz, 1H).

### Example 201

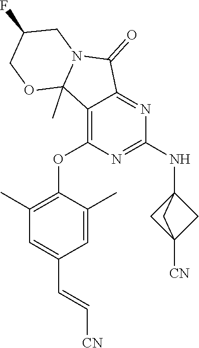 [SMILES: Cc1cc(/C=C/C#N)cc(C)c1Oc1nc(NC23CC(C#N)(C2)C3)nc2c1C1(C)OC[C@@H](F)CN1C2=O]

3-(((7S)-4-(4-((E)-2-cyanovinyl)-2,6-dimethylphenoxy)-7-fluoro-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: (2S)-3-amino-2-fluoro-propan-1-ol was used in place of (R)-3-amino-2-methoxypropan-1-ol, and the mixture was heated to 130° C. for 1 h, then 160° C. for 2 h. MS (m/z) 501.2 [M+H]+. ¹H NMR (400 MHz, DMSO-d6) δ 8.82 (s, 1H), 7.64 (d, J=16.6 Hz, 1H), 7.57 (s, 2H), 6.44 (d, J=16.7 Hz, 1H), 4.90-4.64 (m, 1H), 4.53 (s, 1H), 3.92 (td, J=18.7, 17.1, 10.7 Hz, 2H), 3.45 (s, 1H), 2.11 (s, 3H), 2.04 (s, 3H), 1.96 (m, 9H). ¹⁹F NMR (376 MHz, DMSO-d6) δ −191.57-−191.98 (m).

### Example 202

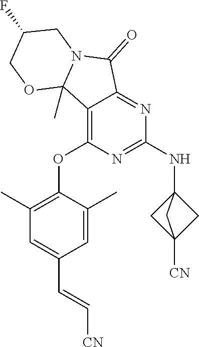 [SMILES: Cc1cc(/C=C/C#N)cc(C)c1Oc1nc(NC23CC(C#N)(C2)C3)nc2c1C1(C)OC[C@H](F)CN1C2=O]

3-(((7R)-4-(4-((E)-2-cyanovinyl)-2,6-dimethylphenoxy)-7-fluoro-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile was prepared using the procedure described for the synthesis of 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235) with the following modifications: (2R)-3-amino-2-fluoro-propan-1-ol was used in place of (R)-3-amino-2-methoxypropan-1-ol, and the mixture was heated to 130° C. for 1 h. MS (m/z) 500.9 [M+H]+. ¹H NMR (400 MHz, DMSO-d6) δ 8.83 (s, 1H), 7.64 (d, J=16.6 Hz, 1H), 7.58 (s, 2H), 6.45 (d, J=16.7 Hz, 1H), 4.87-4.64 (m, 1H), 4.61-4.47 (m, 1H), 4.02-3.81 (m, 2H), 3.51-3.40 (m, 1H), 2.12 (s, 3H), 2.05 (s, 3H), 2.02-1.88 (m, 8H). ¹⁹F NMR (376 MHz, DMSO-d6) δ −191.51-−192.08 (m).

The following compounds were prepared using the procedure described for the synthesis of (E)-3-((4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile (Compound 136), with the following modifications:

### Example 203

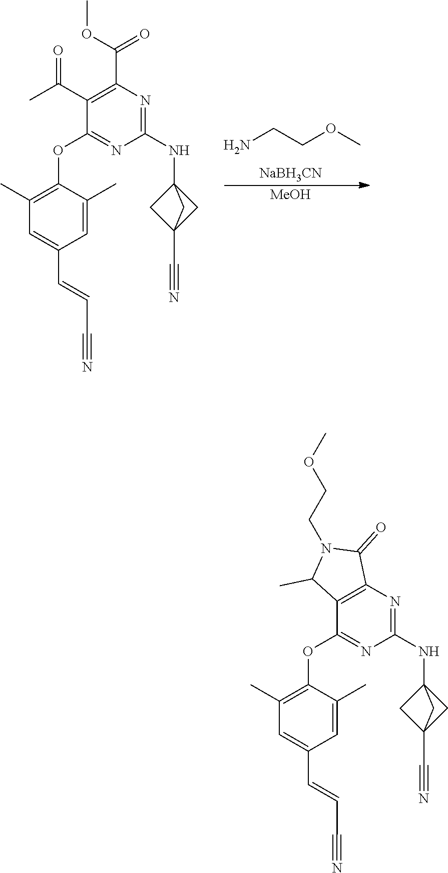

### (E)-3-((4-(4-(2-cyanovinyl)-2,6-dimethylphenoxy)-6-(2-methoxyethyl)-5-methyl-7-oxo-6,7-dihydro-5H-pyrrolo[3,4-d]pyrimidin-2-yl)amino)bicyclo[1.1.1]pentane-1-carbonitrile

2-Methoxyethylamine was used in place of ammonium acetate to afford the title compound as a trifluoroacetic acid salt. MS (m/z) 485.2 [M+H]+. ¹H NMR (400 MHz, Acetonitrile-d3) δ 7.51 (d, J=16.7 Hz, 1H), 7.44 (s, 2H), 6.10 (d, J=16.7 Hz, 1H), 4.85 (q, 1H), 4.09 (ddd, J=14.5, 6.4, 4.4 Hz, 1H), 3.66 (ddd, J=10.6, 6.4, 4.3 Hz, 1H), 3.58 (ddd, J=10.4, 6.5, 4.5 Hz, 1H), 3.43 (ddd, J=14.5, 6.5, 4.3 Hz, 1H), 3.34 (s, 3H), 2.15 (s, 6H), 2.09 (s, 6H), 1.61 (d, J=6.6 Hz, 3H).

### Example 204

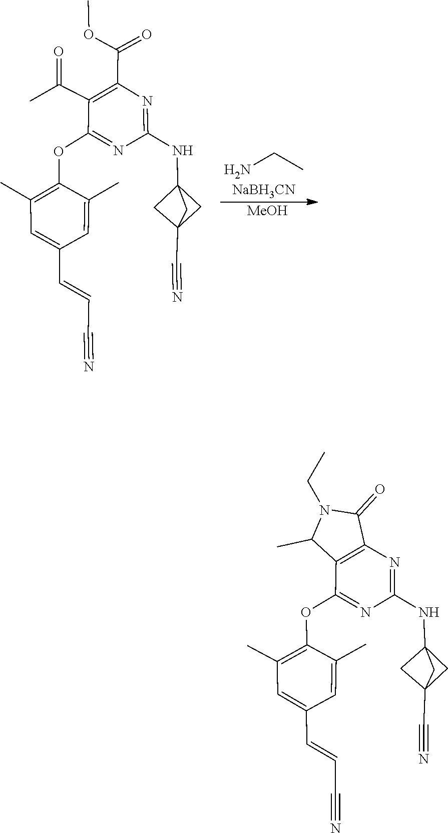

### (E)-3-((4-(4-(2-cyanovinyl)-2,6-dimethylphenoxy)-6-ethyl-5-methyl-7-oxo-6,7-dihydro-5H-pyrrolo[3,4-d]pyrimidin-2-yl)amino)bicyclo[1.1.1]pentane-1-carbonitrile

Ethylamine was used in place of ammonium acetate to afford the title compound as a trifluoroacetic acid salt. MS (m/z) 455.3 [M+H]+. ¹H NMR (400 MHz, Acetonitrile-d3) δ 7.52 (d, J=16.7 Hz, 1H), 7.44 (s, 2H), 6.11 (d, J=16.7 Hz, 1H), 4.81 (q, J=6.6 Hz, 1H), 3.88 (dq, J=14.5, 7.3 Hz, 1H), 3.39 (dq, J=14.2, 7.1 Hz, 1H), 2.15 (s, 6H), 2.09 (s, 6H), 1.62 (d, J=6.6 Hz, 3H), 1.26 (t, J=7.2 Hz, 3H).

### Example 205

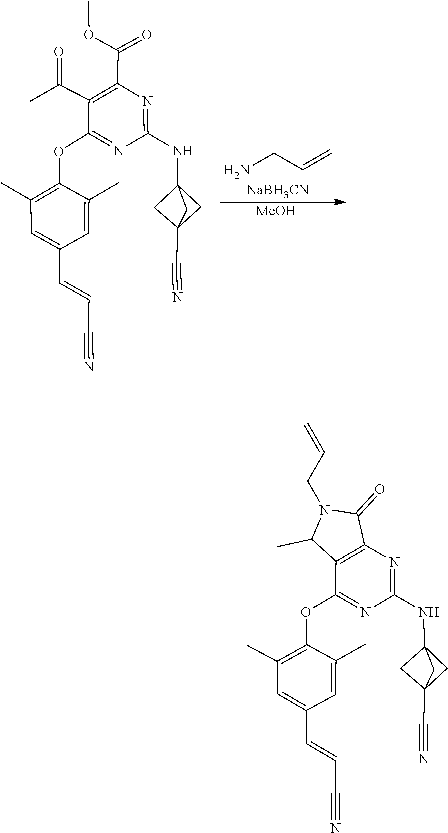

### (E)-3-((6-allyl-4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile

Allylamine was used in place of ammonium acetate to afford the title compound as a trifluoroacetic acid salt. MS (m/z) 467.2 [M+H]+. ¹H NMR (400 MHz, Acetonitrile-d3) δ 7.51 (d, J=16.7 Hz, 1H), 7.43 (s, 2H), 6.81 (s, 1H), 6.10 (d, J=16.7 Hz, 1H), 5.91 (dddd, J=17.0, 10.2, 6.7, 4.9 Hz, 1H), 5.33-5.22 (m, 2H), 4.73 (q, J=6.6 Hz, 1H), 4.49 (ddt, J=15.9, 4.9, 1.8 Hz, 1H), 3.94 (ddt, J=15.9, 6.8, 1.4 Hz, 1H), 2.14 (s, 6H), 2.09 (s, 6H), 1.59 (d, J=6.6 Hz, 3H).

### Example 206

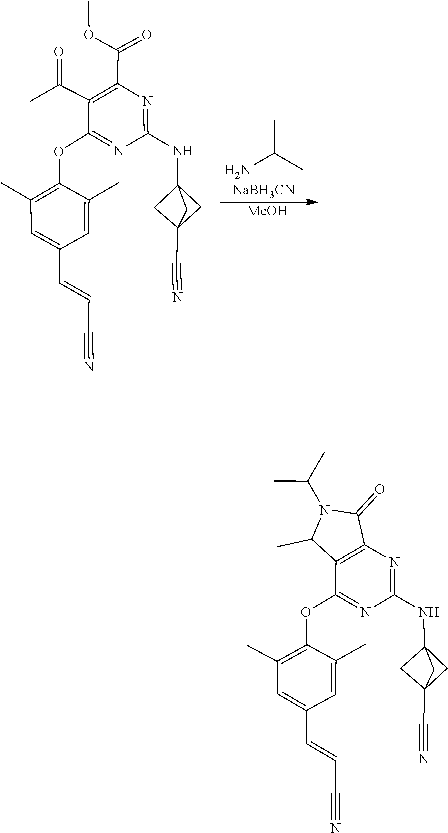

### (E)-3-((4-(4-(2-cyanovinyl)-2,6-dimethylphenoxy)-6-isopropyl-5-methyl-7-oxo-6,7-dihydro-5H-pyrrolo[3,4-d]pyrimidin-2-yl)amino)bicyclo[1.1.1]pentane-1-carbonitrile

Isopropylamine was used in place of ammonium acetate to afford the title compound as a trifluoroacetic acid salt. MS (m/z) 469.3 [M+H]+. ¹H NMR (400 MHz, Acetonitrile-d3) δ 7.51 (d, J=16.7 Hz, 1H), 7.43 (s, 2H), 6.73 (s, 1H), 6.10 (d, J=16.7 Hz, 1H), 4.80 (q, J=6.5 Hz, 1H), 4.22 (hept, J=6.8 Hz, 1H), 2.16-2.01 (m, 12H), 1.66 (d, J=6.5 Hz, 3H), 1.48 (d, J=7.0 Hz, 3H), 1.42 (d, J=6.8 Hz, 3H).

### Example 207

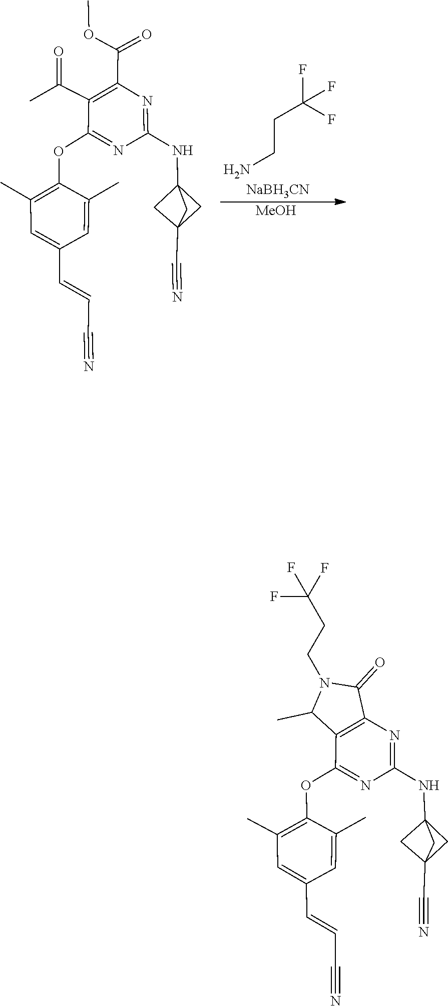

### (E)-3-((4-(4-(2-cyanovinyl)-2,6-dimethylphenoxy)-5-methyl-7-oxo-6-(3,3,3-trifluoropropyl)-6,7-dihydro-5H-pyrrolo[3,4-d]pyrimidin-2-yl)amino)bicyclo[1.1.1]pentane-1-carbonitrile

3,3,3-trifluoropropyl-1-amine was used in place of ammonium acetate to afford the title compound as a trifluoroacetic acid salt. MS (m/z) 523.3 [M+H]+. ¹H NMR (400 MHz, Acetonitrile-d3) δ 7.51 (d, J=16.7 Hz, 1H), 7.43 (s, 2H), 6.81 (s, 1H), 6.10 (d, J=16.7 Hz, 1H), 4.77 (q, J=6.6 Hz, 1H), 4.09 (ddd, J=14.8, 8.4, 6.8 Hz, 1H), 3.58 (ddd, J=14.2, 8.4, 5.7 Hz, 1H), 2.72-2.52 (m, 2H), 2.17-2.03 (m, 12H), 1.62 (d, J=6.6 Hz, 3H).

### Example 208

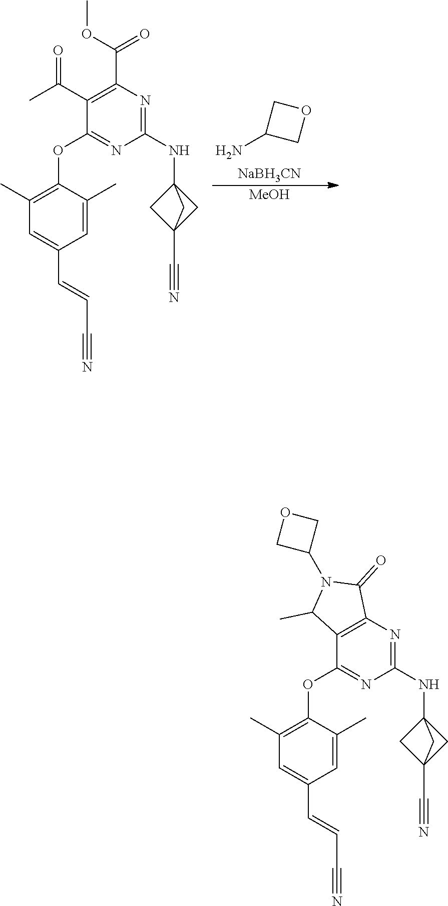

### (E)-3-((4-(4-(2-cyanovinyl)-2,6-dimethylphenoxy)-5-methyl-6-(oxetan-3-yl)-7-oxo-6,7-dihydro-5H-pyrrolo[3,4-d]pyrimidin-2-yl)amino)bicyclo[1.1.1]pentane-1-carbonitrile

3-oxetanamine was used in place of ammonium acetate to afford the title compound as a trifluoroacetic acid salt. MS (m/z) 483.3 [M+H]+. ¹H NMR (400 MHz, Acetonitrile-d3) δ 7.51 (d, J=16.7 Hz, 1H), 7.43 (s, 2H), 6.68 (s, 1H), 6.10 (d, J=16.7 Hz, 1H), 5.15-5.04 (m, 3H), 4.95-4.82 (m, 3H), 2.17-2.03 (m, 12H), 1.62 (d, J=6.6 Hz, 3H).

### Example 209

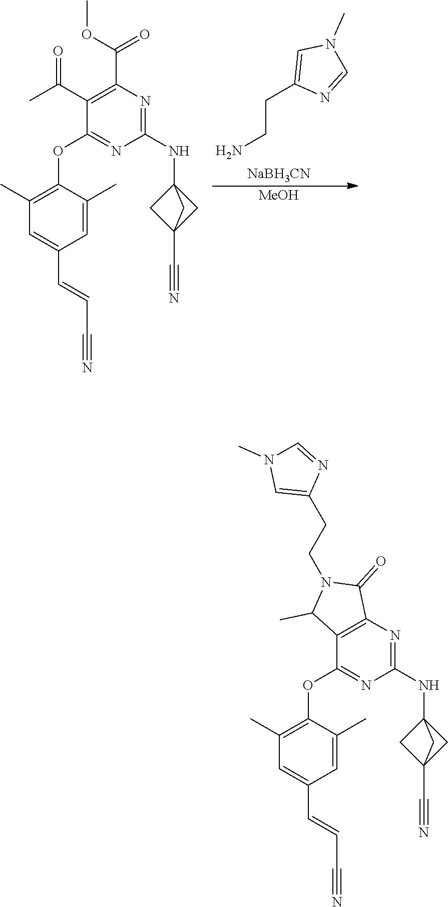

### (E)-3-((4-(4-(2-cyanovinyl)-2,6-dimethylphenoxy)-5-methyl-6-(2-(1-methyl-1H-imidazol-4-yl)ethyl)-7-oxo-6,7-dihydro-5H-pyrrolo[3,4-d]pyrimidin-2-yl)amino)bicyclo[1.1.1]pentane-1-carbonitrile

1-methylhistamine dihydrochloride was used in place of ammonium acetate to afford the title compound as a trifluoroacetic acid salt. MS (m/z) 535.2 [M+H]+. ¹H NMR (400 MHz, Acetonitrile-d3) δ 8.37 (s, 1H), 7.51 (d, J=16.7 Hz, 1H), 7.43 (s, 2H), 7.15 (s, 1H), 6.75 (s, 1H), 6.10 (d, J=16.7 Hz, 1H), 4.73 (q, J=6.6 Hz, 1H), 4.10 (dt, J=14.4, 7.3 Hz, 1H), 3.79 (s, 3H), 3.67 (dt, J=13.7, 6.3 Hz, 1H), 3.11 (q, J=6.5 Hz, 2H), 2.17-2.02 (m, 12H), 1.60 (d, J=6.6 Hz, 3H).

### Example 210

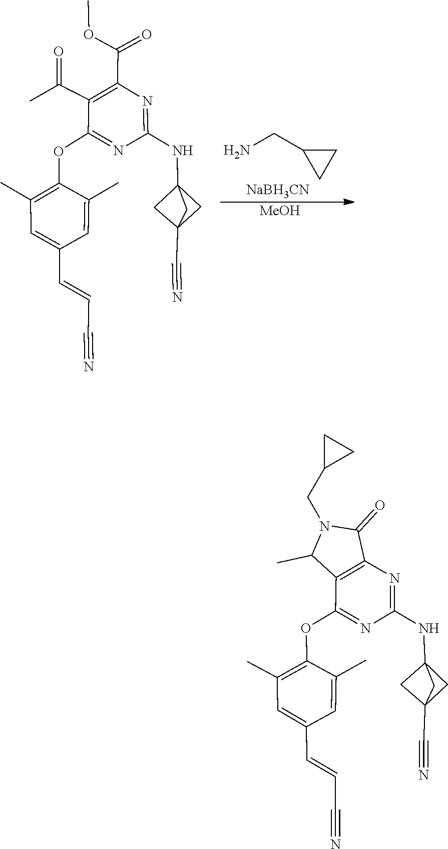

### (E)-3-((4-(4-(2-cyanovinyl)-2,6-dimethylphenoxy)-6-(cyclopropylmethyl)-5-methyl-7-oxo-6,7-dihydro-5H-pyrrolo[3,4-d]pyrimidin-2-yl)amino)bicyclo[1.1.1]pentane-1-carbonitrile

Aminomethylcyclopropane was used in place of ammonium acetate to afford the title compound as a trifluoroacetic acid salt. MS (m/z) 481.2 [M+H]+. ¹H NMR (400 MHz, Acetonitrile-d3) δ 7.88 (s, 1H), 7.52 (d, J=16.7 Hz, 1H), 7.44 (s, 2H), 6.11 (d, J=16.7 Hz, 1H), 4.93 (q, J=6.6 Hz, 1H), 3.81 (dd, J=14.4, 6.8 Hz, 1H), 3.12 (dd, J=14.4, 7.4 Hz, 1H), 2.20-2.01 (m, 12H), 1.63 (d, J=6.6 Hz, 3H), 1.18-1.05 (m, 1H), 0.66-0.58 (m, 1H), 0.58-0.50 (m, 1H), 0.49-0.41 (m, 1H), 0.35-0.27 (m, 1H).

### Example 211

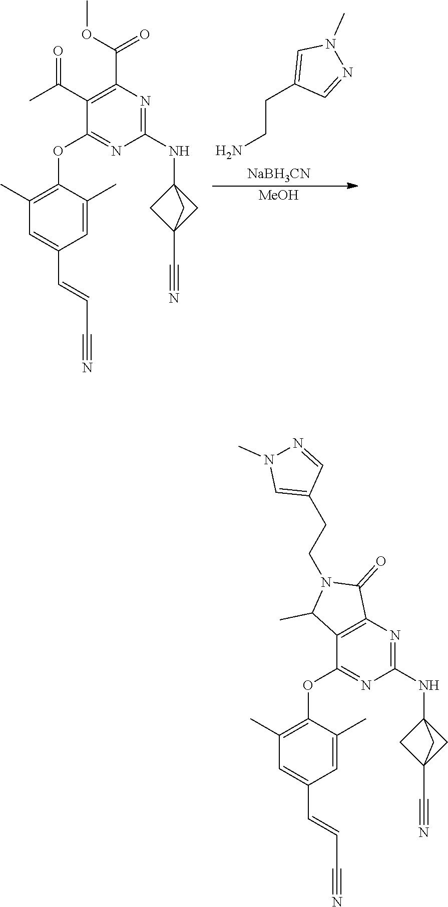

### (E)-3-((4-(4-(2-cyanovinyl)-2,6-dimethylphenoxy)-5-methyl-6-(2-(1-methyl-1H-pyrazol-4-yl)ethyl)-7-oxo-6,7-dihydro-5H-pyrrolo[3,4-d]pyrimidin-2-yl)amino)bicyclo[1.1.1]pentane-1-carbonitrile

2-(2-methylpyrazol-3-yl)ethanamine was used in place of ammonium acetate to afford the title compound as a trifluoroacetic acid salt. MS (m/z) 535.3 [M+H]+. ¹H NMR (400 MHz, DMSO-d6) δ 8.58 (s, 1H), 7.64 (d, J=16.6 Hz, 1H), 7.58 (s, 2H), 7.29 (d, J=1.8 Hz, 1H), 6.45 (d, J=16.7 Hz, 1H), 6.13 (d, J=1.8 Hz, 1H), 4.78 (q, J=6.4 Hz, 1H), 4.01 (dq, J=13.7, 6.8, 5.7 Hz, 1H), 3.79 (s, 3H), 3.53 (ddd, J=14.0, 8.7, 5.3 Hz, 1H), 3.06 (ddd, J=14.4, 8.6, 5.3 Hz, 1H), 2.96 (dt, J=15.4, 7.8 Hz, 1H), 2.09 (s, 6H), 1.97 (s, 6H), 1.52 (d, J=6.5 Hz, 3H).

### Example 212

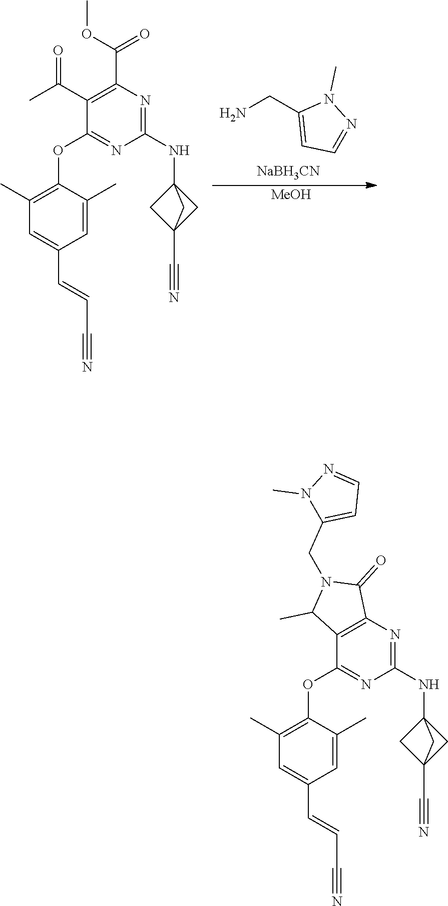

### (E)-3-((4-(4-(2-cyanovinyl)-2,6-dimethylphenoxy)-5-methyl-6-((1-methyl-1H-pyrazol-5-yl)methyl)-7-oxo-6,7-dihydro-5H-pyrrolo[3,4-d]pyrimidin-2-yl)amino)bicyclo[1.1.1]pentane-1-carbonitrile

(1-methyl-1H-pyrazol-5-yl)methylamine was used in place of ammonium acetate to afford the title compound as a trifluoroacetic acid salt. MS (m/z) 521.2 [M+H]+. ¹H NMR (400 MHz, DMSO-d6) δ 8.58 (s, 1H), 7.63 (d, J=16.7 Hz, 1H), 7.56 (s, 2H), 7.37 (d, J=1.8 Hz, 1H), 6.44 (d, J=16.7 Hz, 1H), 6.32 (d, J=1.8 Hz, 1H), 5.09 (d, J=16.0 Hz, 1H), 4.63-4.54 (m, 2H), 3.81 (s, 3H), 2.08 (s, 6H), 1.97 (s, 6H), 1.54 (d, J=6.5 Hz, 3H).

### Example 213

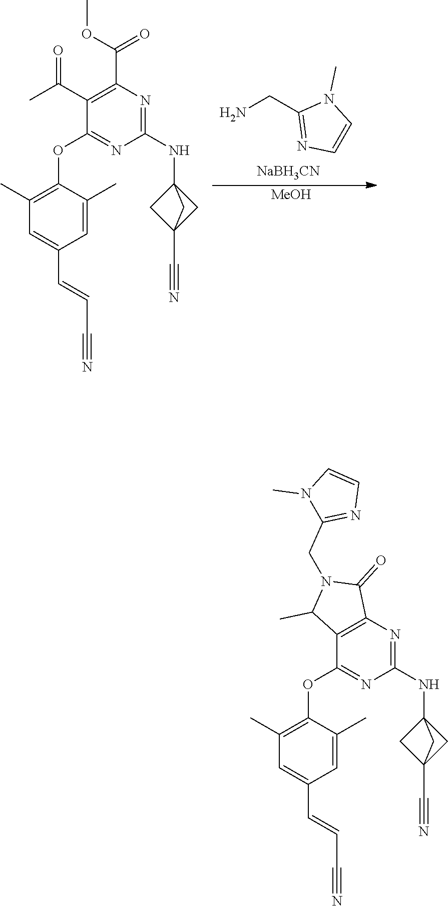

### (E)-3-((4-(4-(2-cyanovinyl)-2,6-dimethylphenoxy)-5-methyl-6-((1-methyl-1H-imidazol-2-yl)methyl)-7-oxo-6,7-dihydro-5H-pyrrolo[3,4-d]pyrimidin-2-yl)amino)bicyclo[1.1.1]pentane-1-carbonitrile

1-(1-methyl-1H-imidazol-2-yl)methylamine was used in place of ammonium acetate to afford the title compound as a trifluoroacetic acid salt. MS (m/z) 521.3 [M+H]+. ¹H NMR (400 MHz, DMSO-d6) δ 8.58 (s, 1H), 7.63 (d, J=16.7 Hz, 1H), 7.56 (s, 2H), 7.13 (d, J=1.2 Hz, 1H), 6.83 (d, J=1.2 Hz, 1H), 6.44 (d, J=16.7 Hz, 1H), 5.10 (d, J=15.8 Hz, 1H), 4.67 (q, J=6.6 Hz, 1H), 4.54 (d, J=15.8 Hz, 1H), 3.64 (s, 3H), 2.08 (s, 6H), 1.97 (s, 6H), 1.54 (d, J=6.6 Hz, 3H).

### Example 214

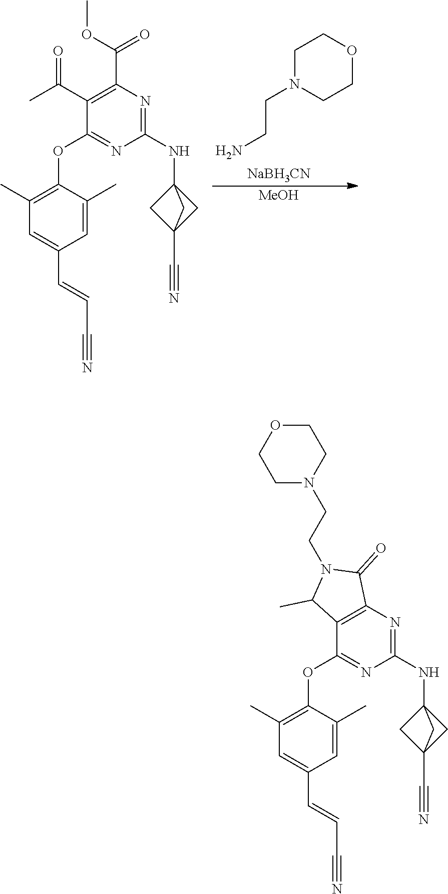

### (E)-3-((4-(4-(2-cyanovinyl)-2,6-dimethylphenoxy)-5-methyl-6-(2-morpholinoethyl)-7-oxo-6,7-dihydro-5H-pyrrolo[3,4-d]pyrimidin-2-yl)amino)bicyclo[1.1.1]pentane-1-carbonitrile

4-(2-aminoethyl)morpholine was used in place of ammonium acetate to afford the title compound as a trifluoroacetic acid salt. MS (m/z) 540.3 [M+H]+. ¹H NMR (400 MHz, Acetonitrile-d3) δ 7.52 (d, J=16.7 Hz, 1H), 7.43 (s, 2H), 6.88 (s, 1H), 6.10 (d, J=16.7 Hz, 1H), 4.85 (q, J=6.6 Hz, 1H), 4.28-3.67 (m, 8H), 3.66-3.58 (m, 1H), 3.52-3.42 (m, 1H), 3.37-3.27 (m, 1H), 3.10 (s, 1H), 2.18-2.01 (m, 12H), 1.63 (d, J=6.6 Hz, 3H).

### Example 215

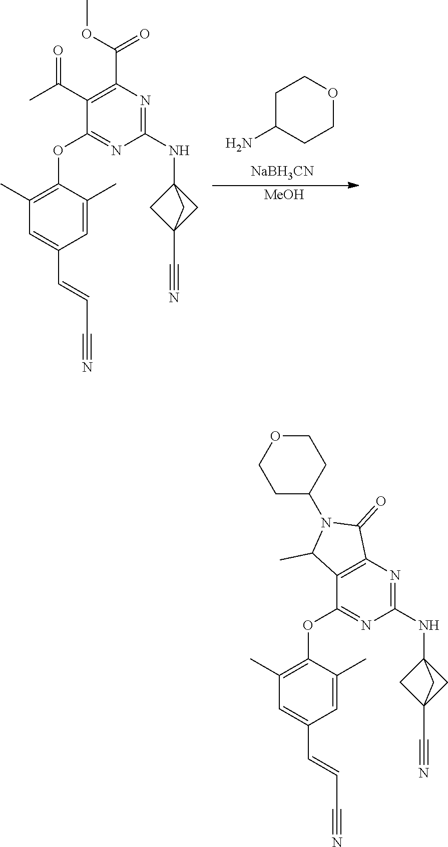

### (E)-3-((4-(4-(2-cyanovinyl)-2,6-dimethylphenoxy)-5-methyl-7-oxo-6-(tetrahydro-2H-pyran-4-yl)-6,7-dihydro-5H-pyrrolo[3,4-d]pyrimidin-2-yl)amino)bicyclo[1.1.1]pentane-1-carbonitrile

4-Aminotetrahydropyran was used in place of ammonium acetate to afford the title compound as a trifluoroacetic acid salt. MS (m/z) 511.3 [M+H]+. ¹H NMR (400 MHz, Acetonitrile-d3) δ 7.51 (d, J=16.7 Hz, 1H), 7.44 (s, 2H), 6.10 (d, J=16.7 Hz, 1H), 4.83 (q, J=6.5 Hz, 1H), 4.08-3.96 (m, 3H), 3.48 (tdd, J=11.6, 9.1, 2.1 Hz, 2H), 2.40 (qd, J=12.4, 4.8 Hz, 1H), 2.26 (qd, J=12.3, 4.5 Hz, 1H), 2.14 (s, 6H), 2.09 (s, 6H), 1.76 (tt, J=3.5 Hz, 2H), 1.68 (d, J=6.5 Hz, 3H).

### Example 216

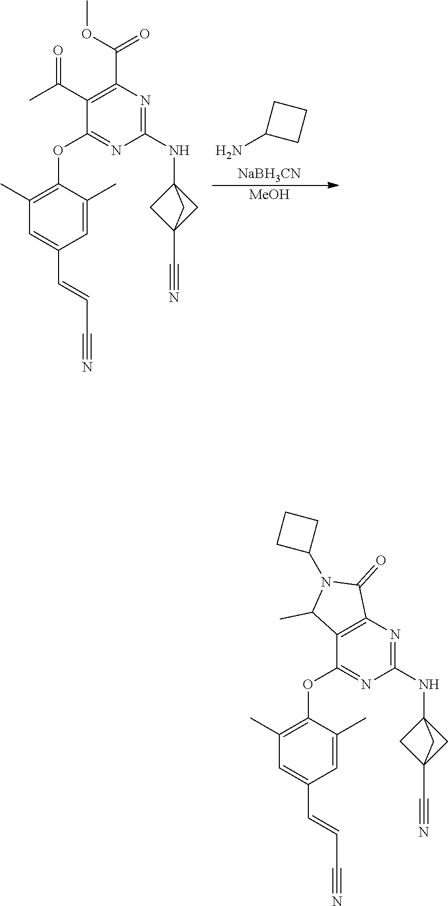

### (E)-3-((4-(4-(2-cyanovinyl)-2,6-dimethylphenoxy)-6-cyclobutyl-5-methyl-7-oxo-6,7-dihydro-5H-pyrrolo[3,4-d]pyrimidin-2-yl)amino)bicyclo[1.1.1]pentane-1-carbonitrile

Cyclobutylamine was used in place of ammonium acetate to afford the title compound as a trifluoroacetic acid salt. MS (m/z) 481.3 [M+H]+. ¹H NMR (400 MHz, Acetonitrile-d₃) δ 7.51 (d, J=16.7 Hz, 1H), 7.43 (s, 2H), 6.10 (d, J=16.7 Hz, 1H), 4.84 (q, J=6.5 Hz, 1H), 4.41 (tt, J=8.8, 7.6 Hz, 1H), 2.76-2.54 (m, 2H), 2.43-2.25 (m, 2H), 2.14 (s, 6H), 2.08 (s, 6H), 1.90-1.81 (m, 2H), 1.63 (d, J=6.6 Hz, 3H).

### Example 217

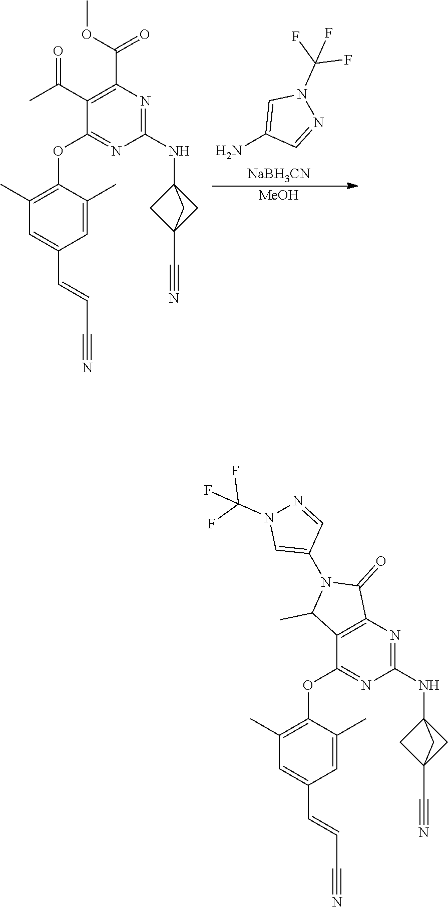

### (E)-3-((4-(4-(2-cyanovinyl)-2,6-dimethylphenoxy)-5-methyl-7-oxo-6-(1-(trifluoromethyl)-1H-pyrazol-4-yl)-6,7-dihydro-5H-pyrrolo[3,4-d]pyrimidin-2-yl)amino)bicyclo[1.1.1]pentane-1-carbonitrile

1-(trifluoromethyl)pyrazol-4-amine was used in place of ammonium acetate to afford the title compound as a trifluoroacetic acid salt. MS (m/z) 561.3 [M+H]+. ¹H NMR (400 MHz, Acetonitrile-d₃) δ 8.53 (s, 1H), 8.27 (s, 1H), 7.52 (d, J=16.7 Hz, 1H), 7.45 (s, 2H), 6.76 (s, 1H), 6.11 (d, J=16.7 Hz, 1H), 5.22 (q, J=6.5 Hz, 1H), 2.21-2.04 (m, 12H), 1.69 (d, J=6.4 Hz, 3H). ¹⁹F NMR (376 MHz, CD₃CN) δ −61.83, −77.29.

### Example 218

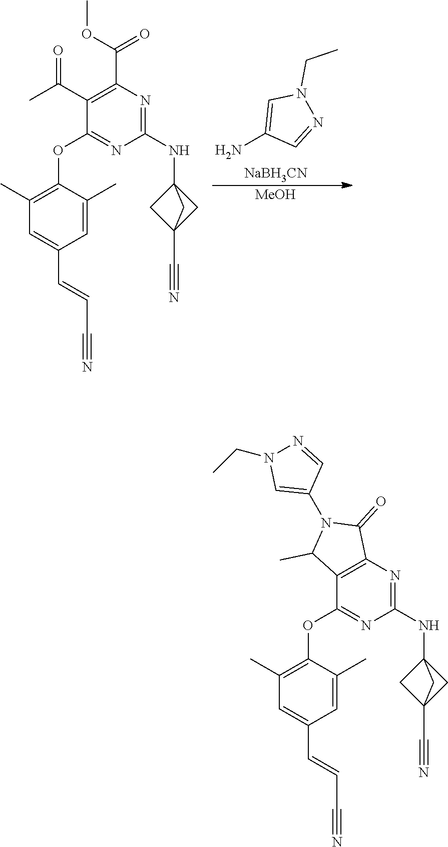

### (E)-3-((4-(4-(2-cyanovinyl)-2,6-dimethylphenoxy)-6-(1-ethyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-ethylpyrazol-4-amine was used in place of ammonium acetate to afford the title compound as a trifluoroacetic acid salt. MS (m/z) 521.3 [M+H]+. ¹H NMR (400 MHz, Acetonitrile-d3) δ 8.08 (s, 1H), 7.75 (s, 1H), 7.52 (d, J=16.7 Hz, 1H), 7.44 (s, 2H), 6.89 (s, 1H), 6.11 (d, J=16.6 Hz, 1H), 5.14 (q, J=6.5 Hz, 1H), 4.22 (q, J=7.3 Hz, 2H), 2.16 (s, 6H), 2.10 (s, 6H), 1.67 (d, J=6.5 Hz, 3H), 1.48 (t, J=7.3 Hz, 3H).

### Example 219

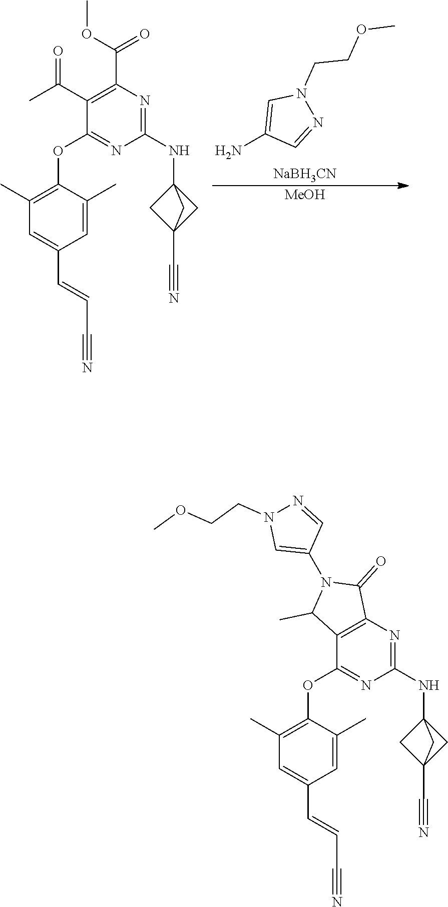

### (E)-3-((4-(4-(2-cyanovinyl)-2,6-dimethylphenoxy)-6-(1-(2-methoxyethyl)-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-(2-methoxyethyl)pyrazol-4-amine was used in place of ammonium acetate to afford the title compound as a trifluoroacetic acid salt. MS (m/z) 551.3 [M+H]+. ¹H NMR (400 MHz, Acetonitrile-d₃) δ 8.10 (s, 1H), 7.76 (s, 1H), 7.52 (d, J=16.7 Hz, 1H), 7.44 (s, 2H), 6.11 (d, J=16.7 Hz, 1H), 5.14 (q, J=6.5 Hz, 1H), 4.32 (t, J=5.2 Hz, 2H), 3.78 (t, J=5.2 Hz, 2H), 3.32 (s, 3H), 2.16 (s, 6H), 2.10 (s, 6H), 1.67 (d, J=6.5 Hz, 3H).

### Example 220

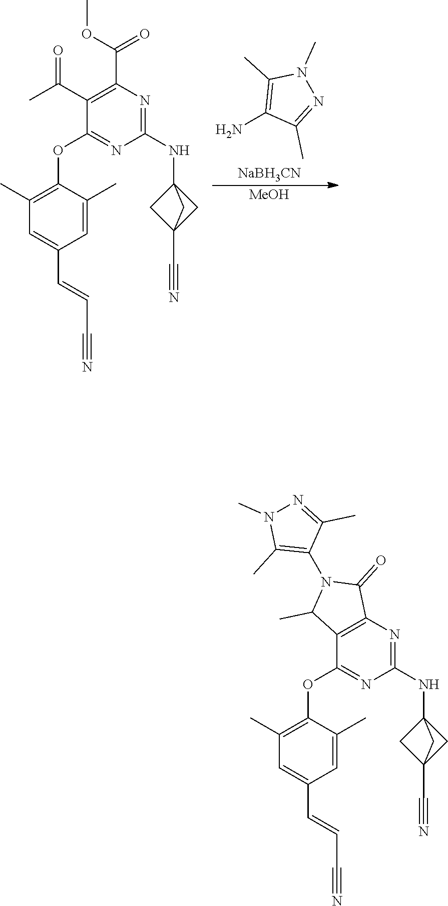

### (E)-3-((4-(4-(2-cyanovinyl)-2,6-dimethylphenoxy)-5-methyl-7-oxo-6-(1,3,5-trimethyl-1H-pyrazol-4-yl)-6,7-dihydro-5H-pyrrolo[3,4-d]pyrimidin-2-yl)amino)bicyclo[1.1.1]pentane-1-carbonitrile

1,3,5-trimethylpyrazol-4-amine was used in place of ammonium acetate to afford the title compound as a trifluoroacetic acid salt. MS (m/z) 535.3 [M+H]+. ¹H NMR (400 MHz, Acetonitrile-d3) δ 7.52 (d, J=16.7 Hz, 1H), 7.45 (s, 2H), 7.07 (s, 1H), 6.11 (d, J=16.7 Hz, 1H), 4.97 (q, J=6.7 Hz, 1H), 3.78 (s, 3H), 2.21-2.06 (m, 18H), 1.48 (d, J=6.6 Hz, 3H).

### Example 221

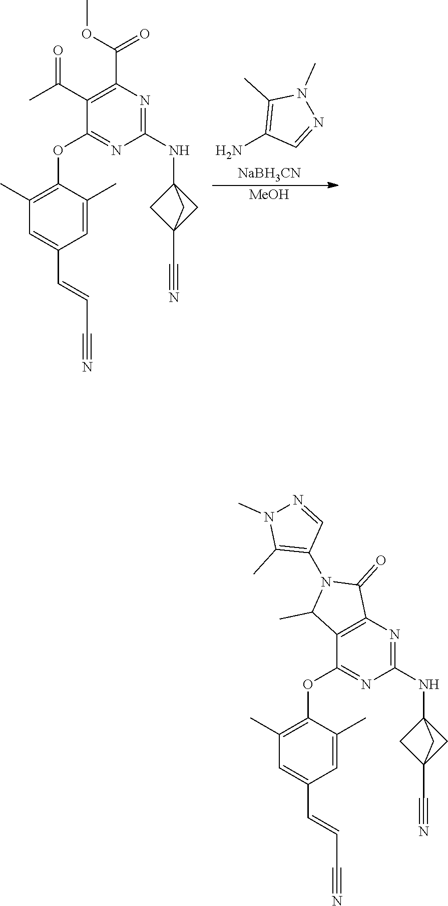

### (E)-3-((4-(4-(2-cyanovinyl)-2,6-dimethylphenoxy)-6-(1,5-dimethyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1,5-dimethylpyrazol-4-amine was used in place of ammonium acetate to afford the title compound as a trifluoroacetic acid salt. MS (m/z) 521.3 [M+H]+. ¹H NMR (400 MHz, Acetonitrile-d3) δ 7.52 (d, J=16.7 Hz, 1H), 7.44 (s, 2H), 7.42 (s, 1H), 6.90 (s, 1H), 6.11 (d, J=16.7 Hz, 1H), 4.97 (q, J=6.6 Hz, 1H), 3.82 (s, 3H), 2.20 (s, 3H), 2.16 (s, 6H), 2.11 (s, 6H), 1.51 (d, J=6.6 Hz, 3H).

### Example 222

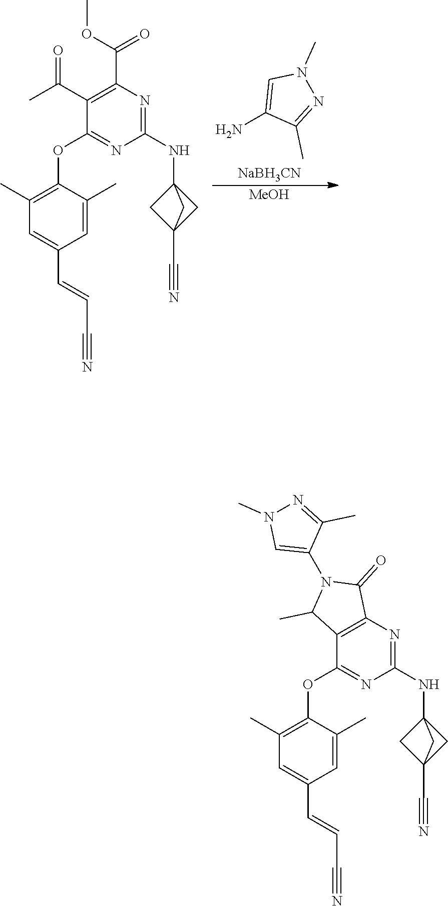

### (E)-3-((4-(4-(2-cyanovinyl)-2,6-dimethylphenoxy)-6-(1,3-dimethyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1,3-dimethylpyrazol-4-amine was used in place of ammonium acetate to afford the title compound as a trifluoroacetic acid salt. MS (m/z) 521.3 [M+H]+. ¹H NMR (400 MHz, Acetonitrile-d3) δ 7.59 (s, 1H), 7.52 (d, J=16.7 Hz, 1H), 7.44 (s, 2H), 6.93 (s, 1H), 6.11 (d, J=16.7 Hz, 1H), 4.96 (q, J=6.6 Hz, 1H), 3.85 (s, 3H), 2.16 (s, 6H), 2.15 (s, 3H), 2.11 (s, 6H), 1.52 (d, J=6.6 Hz, 3H).

### Example 223

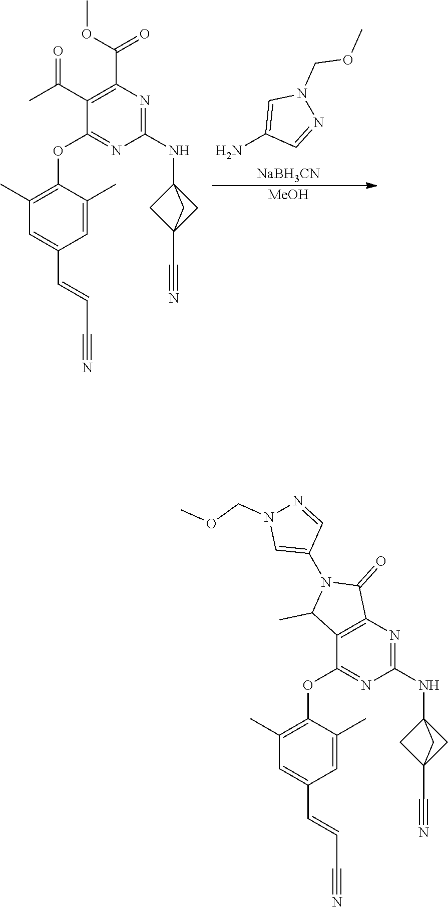

### (E)-3-((4-(4-(2-cyanovinyl)-2,6-dimethylphenoxy)-6-(1-(methoxymethyl)-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-(methoxymethyl)pyrazol-4-amine was used in place of ammonium acetate to afford the title compound as a trifluoroacetic acid salt. MS (m/z) 537.3 [M+H]+. ¹H NMR (400 MHz, Acetonitrile-d3) δ 8.23 (s, 1H), 7.88 (s, 1H), 7.52 (d, J=16.7 Hz, 1H), 7.45 (s, 2H), 6.79 (s, 1H), 6.11 (d, J=16.7 Hz, 1H), 5.42 (s, 2H), 5.17 (q, J=6.4 Hz, 1H), 3.34 (s, 3H), 2.17 (s, 6H), 2.11 (s, 6H), 1.68 (d, J=6.5 Hz, 3H).

### Example 224

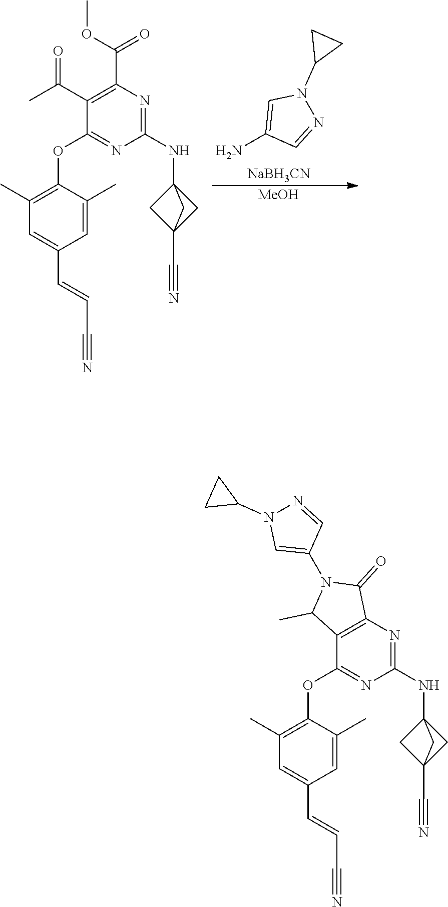

### (E)-3-((4-(4-(2-cyanovinyl)-2,6-dimethylphenoxy)-6-(1-cyclopropyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-cyclopropyl-1H-pyrazol-4-amine was used in place of ammonium acetate to afford the title compound as a trifluoroacetic acid salt. MS (m/z) 533.3 [M+H]+. [M+H]+. ¹H NMR (400 MHz, Acetonitrile-d3) δ 8.09 (s, 1H), 7.74 (s, 1H), 7.52 (d, J=16.7 Hz, 1H), 7.44 (s, 2H), 6.84 (s, 1H), 6.11 (d, J=16.7 Hz, 1H), 5.12 (q, J=6.5 Hz, 1H), 3.71 (tt, J=7.4, 3.8 Hz, 1H), 2.16 (s, 6H), 2.10 (s, 6H), 1.65 (d, J=6.5 Hz, 3H), 1.16-1.09 (m, 2H), 1.09-1.00 (m, 2H).

### Example 225

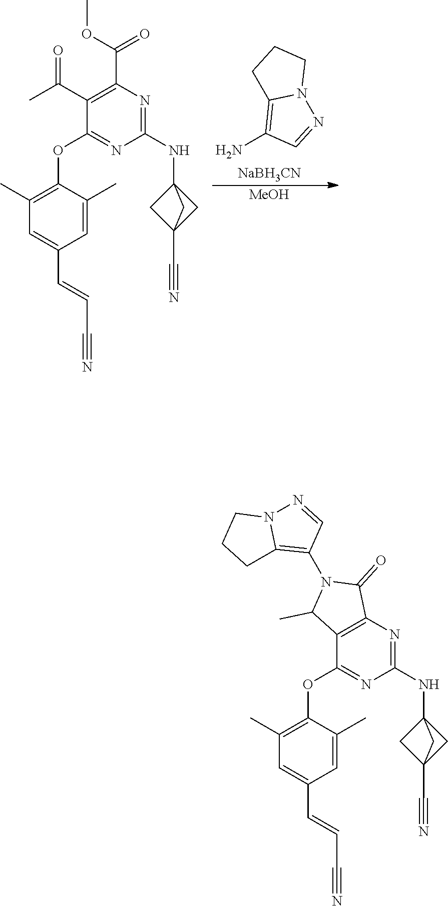

### (E)-3-((4-(4-(2-cyanovinyl)-2,6-dimethylphenoxy)-6-(5,6-dihydro-4H-pyrrolo[1,2-b]pyrazol-3-yl)-5-methyl-7-oxo-6,7-dihydro-5H-pyrrolo[3,4-d]pyrimidin-2-yl)amino)bicyclo[1.1.1]pentane-1-carbonitrile

5,6-dihydro-4H-pyrrolo[1,2-b]pyrazol-3-amine was used in place of ammonium acetate to afford the title compound as a trifluoroacetic acid salt. MS (m/z) 533.3 [M+H]+. ¹H NMR (400 MHz, DMSO) δ 8.62 (s, 1H), 7.73 (s, 1H), 7.65 (d, J=16.7 Hz, 1H), 7.59 (s, 2H), 6.45 (d, J=16.7 Hz, 1H), 5.27-5.19 (m, 1H), 4.13 (t, J=7.2 Hz, 2H), 3.11 (dt, J=15.3, 7.3 Hz, 1H), 2.93 (ddd, J=15.5, 8.7, 6.0 Hz, 1H), 2.59 (dt, J=13.8, 6.9 Hz, 2H), 2.15-2.08 (m, 6H), 1.99 (s, 6H), 1.50 (d, J=6.5 Hz, 3H).

### Example 226

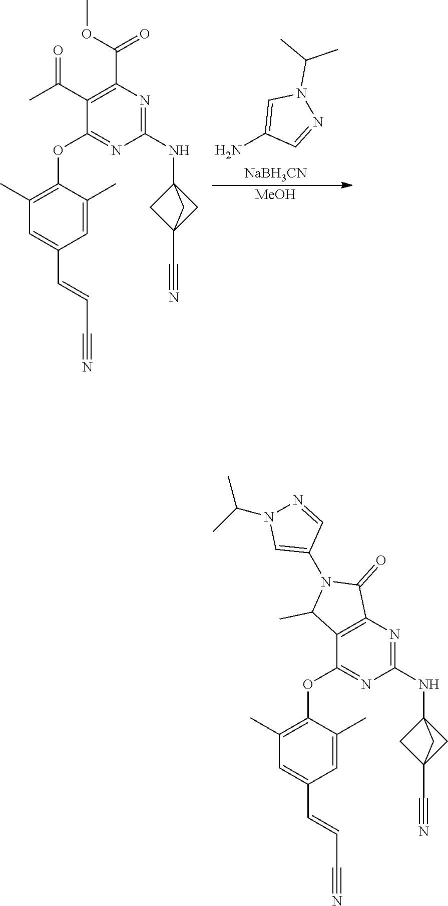

### (E)-3-((4-(4-(2-cyanovinyl)-2,6-dimethylphenoxy)-6-(1-isopropyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-isopropylpyrazol-4-amine was used in place of ammonium acetate to afford the title compound as a trifluoroacetic acid salt. MS (m/z) 535.3 [M+H]+. ¹H NMR (400 MHz, Acetonitrile-d3) δ 8.09 (s, 1H), 7.76 (s, 1H), 7.52 (d, J=16.7 Hz, 1H), 7.44 (s, 2H), 6.84 (s, 1H), 6.11 (d, J=16.7 Hz, 1H), 5.14 (q, J=6.4 Hz, 1H), 4.58 (dq, J=13.4, 6.8 Hz, 1H), 2.16 (s, 6H), 2.10 (s, 6H), 1.66 (d, J=6.5 Hz, 3H), 1.52 (d, J=6.7 Hz, 6H).

### Example 227

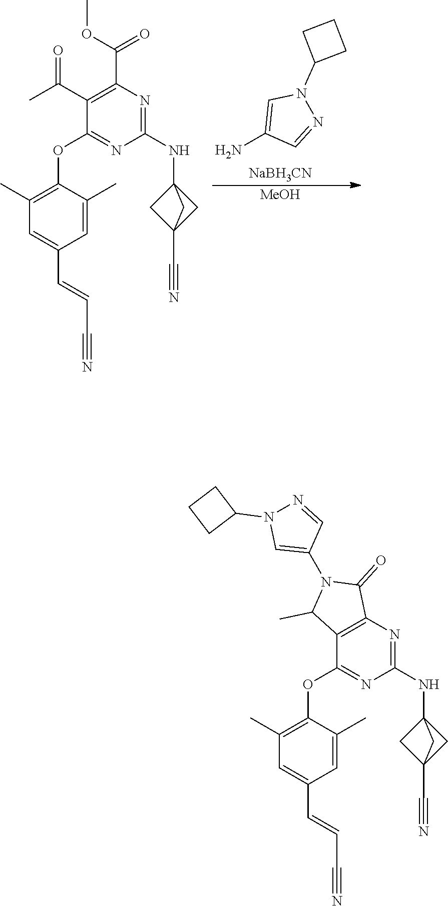

### (E)-3-((4-(4-(2-cyanovinyl)-2,6-dimethylphenoxy)-6-(1-cyclobutyl-1H-pyrazol-4-yl)-5-methyl-7-oxo-6,7-dihydro-5H-pyrrolo[3,4-d]pyrimidin-2-yl)amino)bicyclo[1.1.1]pentane-1-carbonitrile

1-cyclobutylpyrazol-4-amine was used in place of ammonium acetate to afford the title compound as a trifluoroacetic acid salt. MS (m/z) 547.3 [M+H]+. ¹H NMR (400 MHz, Acetonitrile-d3) δ 8.12 (s, 1H), 7.79 (s, 1H), 7.52 (d, J=16.7 Hz, 1H), 7.45 (s, 2H), 7.01 (s, 1H), 6.11 (d, J=16.7 Hz, 1H), 5.19-5.09 (m, 1H), 4.94-4.80 (m, 1H), 2.64-2.42 (m, 4H), 2.19-2.05 (m, 12H), 1.95-1.81 (m, 3H), 1.66 (d, J=6.5 Hz, 3H).

### Example 228

### (S,E)-3-((4-(4-(2-cyanovinyl)-2,6-dimethylphenoxy)-5-methyl-7-oxo-6-(1H-pyrazol-4-yl)-6,7-dihydro-5H-pyrrolo[3,4-d]pyrimidin-2-yl)amino)bicyclo[1.1.1]pentane-1-carbonitrile

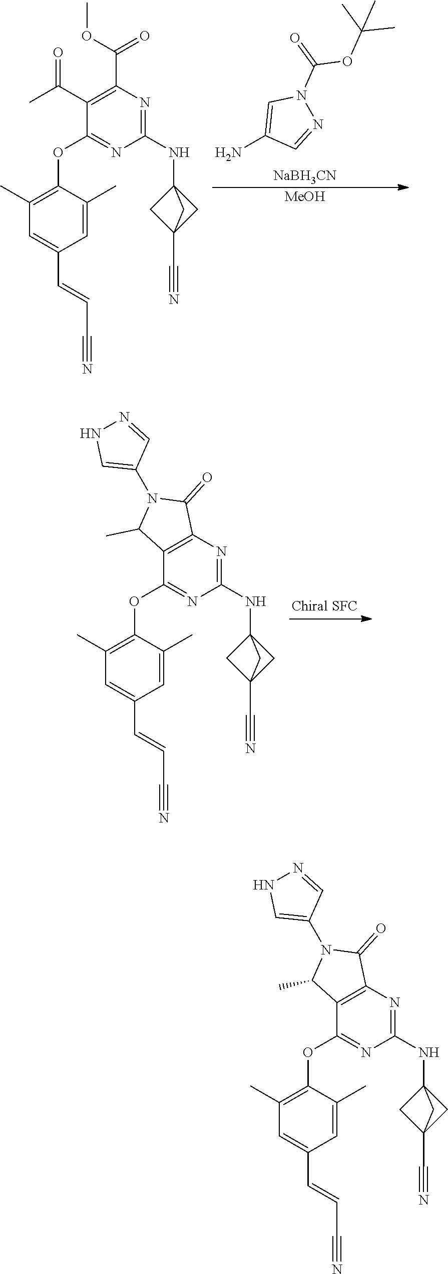

### Step 1, Preparation of (E)-3-((4-(4-(2-cyanovinyl)-2,6-dimethylphenoxy)-5-methyl-7-oxo-6-(1H-pyrazol-4-yl)-6,7-dihydro-5H-pyrrolo[3,4-d]pyrimidin-2-yl)amino)bicyclo[1.1.1]pentane-1-carbonitrile (Intermediate 228a)

tert-butyl 4-aminopyrazole-1-carboxylate was used in place of ammonium acetate to yield Intermediate 228a as a bis-trifluoroacetic acid salt. MS (m/z) 493.3 [M+H]+. ¹H NMR (400 MHz, DMSO) δ 8.65 (s, 1H), 8.05 (s, 2H), 7.65 (d, J=16.6 Hz, 1H), 7.59 (s, 2H), 6.46 (d, J=16.7 Hz, 1H), 5.29 (q, J=6.4 Hz, 1H), 4.08 (s, 1H), 2.12 (s, 6H), 1.99 (s, 6H), 1.57 (d, J=6.4 Hz, 3H).

### Step 2, Preparation (S,E)-3-((4-(4-(2-cyanovinyl)-2,6-dimethylphenoxy)-5-methyl-7-oxo-6-(1H-pyrazol-4-yl)-6,7-dihydro-5H-pyrrolo[3,4-d]pyrimidin-2-yl)amino)bicyclo[1.1.1]pentane-1-carbonitrile (Compound 228)

Intermediate 228a (14 mg, 0.093 mmol) was subject to chiral SFC (Column 1K, 4.6×100 mm, 5 mic, 50% EtOH/TFA co-solvent, 40° C.) to yield the title compound as a bis-trifluoroacetic acid salt. MS (m/z) 493.3 [M+H]+. ¹H NMR (400 MHz, DMSO) δ 8.65 (s, 1H), 8.05 (s, 2H), 7.65 (d, J=16.6 Hz, 1H), 7.59 (s, 2H), 6.46 (d, J=16.7 Hz, 1H), 5.29 (q, J=6.4 Hz, 1H), 4.08 (s, 1H), 2.12 (s, 6H), 1.99 (s, 6H), 1.57 (d, J=6.4 Hz, 3H).

### Example 229

### (E)-3-((4-(4-(2-cyanovinyl)-2,6-dimethylphenoxy)-5-(difluoromethyl)-5-hydroxy-7-oxo-6,7-dihydro-5H-pyrrolo[3,4-d]pyrimidin-2-yl)amino)bicyclo[1.1.1]pentane-1-carbonitrile

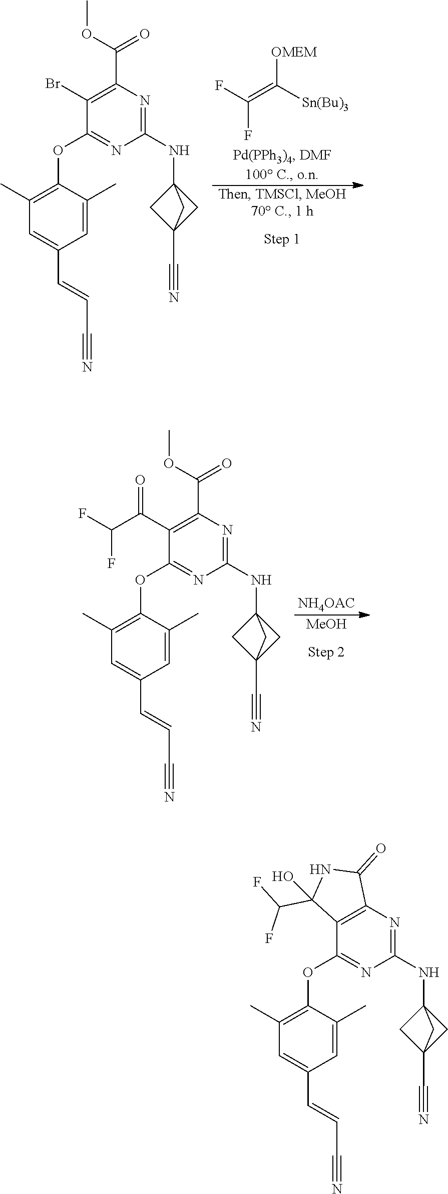

### Step 1, Preparation of methyl (E)-2-((3-cyanobicyclo[1.1.1]pentan-1-yl)amino)-6-(4-(2-cyanovinyl)-2,6-dimethylphenoxy)-5-(2,2-difluoroacetyl)pyrimidine-4-carboxylate (Intermediate 229a)

Intermediate 136c (0.506 mmol, 250 mg) and tetrakis(triphenylphosphine)palladium(0) (0.506 mmol, 584 mg) were suspended in DMF (5 mL). Tributyl-[2,2-difluoro-1-(2-methoxyethoxymethoxy)vinyl]stannane (1.01 mmol, 0.323 mL) was added to the mixture. The mixture was stirred at 100° C. under an argon atmosphere overnight. The mixture was quenched with water and diluted with EtOAc. The aqueous phase was extracted twice with EtOAc. The combined organic phase was washed with brine, dried over MgSO₄, filtered, and concentrated in vacuo. The residue was purified via silica gel flash column chromatography (0-100% EtOAc in hexanes). Then, the product was redissolved in methanol and TMSCl (0.506 mmol, 0.0642 mL) was added. The mixture was stirred at 70° C. for 1 hour and then, concentrated in vacuo to afford crude Intermediate 229a. MS (m/z) 494.2 [M+H]+.

### Step 2, Preparation of (E)-3-((4-(4-(2-cyanovinyl)-2,6-dimethylphenoxy)-5-(difluoromethyl)-5-hydroxy-7-oxo-6,7-dihydro-5H-pyrrolo[3,4-d]pyrimidin-2-yl)amino)bicyclo[1.1.1]pentane-1-carbonitrile (Intermediate 229b)

Intermediate 229a (0.0608 mmol, 30 mg) and ammonium acetate (0.608 mmol, 46.9 mg) were suspended in MeOH (0.5 mL). The mixture was subject to microwave irradiation at 130° C. for 3 hours. The mixture was then concentrated in vacuo, redissolved in DMF, filtered, and purified via preparative HPLC (0-100% MeCN in water, 0.1% TFA) then lyophilized to afford the title compound as the bis-trifluoroacetic acid salt. MS (m/z) 479.2 [M+H]+. ¹H NMR (400 MHz, Acetonitrile-d3) δ 7.83 (s, 1H), 7.51 (d, J=16.7 Hz, 1H), 7.43 (s, 2H), 6.92 (s, 1H), 6.33 (t, J=55.2 Hz, 1H), 6.10 (d, J=16.7 Hz, 1H), 2.16 (s, 3H), 2.13 (s, 3H), 2.06 (s, 6H).

### Example 230

### (E)-3-((4-(4-(2-cyanovinyl)-2,6-dimethylphenoxy)-5,5-dimethyl-6-(1-methyl-1H-pyrazol-4-yl)-7-oxo-6,7-dihydro-5H-pyrrolo[3,4-d]pyrimidin-2-yl)amino)bicyclo[1.1.1]pentane-1-carbonitrile

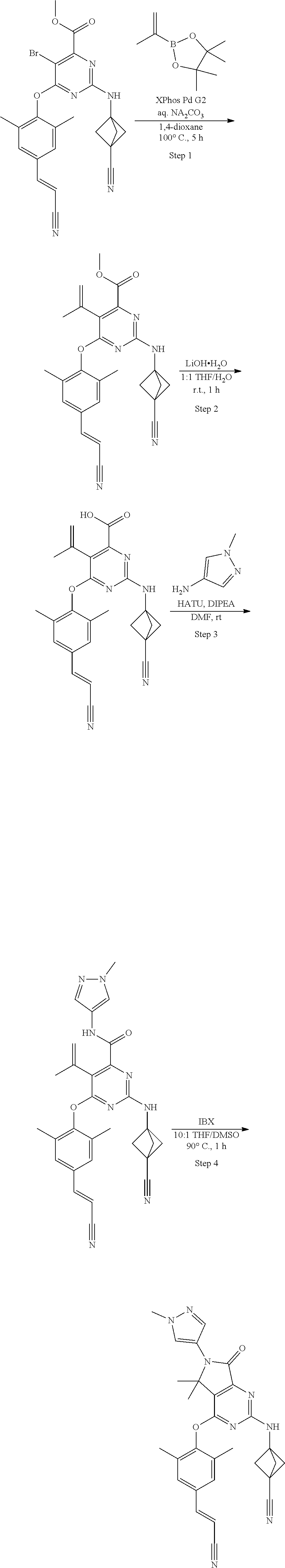

### Step 1, Preparation of Methyl (E)-2-((3-cyanobicyclo[1.1.1]pentan-1-yl)amino)-6-(4-(2-cyanovinyl)-2,6-dimethylphenoxy)-5-(prop-1-en-2-yl)pyrimidine-4-carboxylate (Intermediate 230a)

Intermediate 136c (0.506 mmol, 250 mg) and XPhos Pd G2 (0.253 mmol, 190 mg) were suspended in dioxane (3 mL). 2-isopropenyl-4,4,5,5-tetramethyl-1,3,2-dioxaborolane (1.26 mmol, 0.233 mL) was added and the mixture was stirred at 100° C. under an argon atmosphere for 5 hours. The mixture was passed through celite, dried over MgSO₄, and concentrated in vacuo. The residue was subjected to silica gel flash column chromatography (0-100% EtOAc in hexanes) then concentrated in vacuo to afford Intermediate 230a. MS (m/z) 456.2 [M+H]+.

### Step 2, Preparation of (E)-2-((3-cyanobicyclo[1.1.1]pentan-1-yl)amino)-6-(4-(2-cyanovinyl)-2,6-dimethylphenoxy)-5-(prop-1-en-2-yl)pyrimidine-4-carboxylic acid (Intermediate 230b)

To a solution of Intermediate 230a (0.498 mmol, 227 mg) in 1:1 THF/H₂O (3 mL) was added lithium hydroxide monohydrate (0.997 mmol, 41.8 mg). The mixture was stirred at ambient temperatures for 1 hour. After treating the mixture with 1M aq. HCl until the pH<4, the aqueous phase was extracted with EtOAc twice, dried over MgSO₄, filtered, and concentrated in vacuo to afford Intermediate 230b. MS (m/z) 442.2 [M+H]+.

### Step 3, Preparation of (E)-2-((3-cyanobicyclo[1.1.1]pentan-1-yl)amino)-6-(4-(2-cyanovinyl)-2,6-dimethylphenoxy)-N-(1-methyl-1H-pyrazol-4-yl)-5-(prop-1-en-2-yl)pyrimidine-4-carboxamide (Intermediate 230c)

Intermediate 230b (0.498 mmol, 220 mg), HATU (0.598 mmol, 227 mg), 1-methyl-1H-pyrazol-4-amine (0.997 mmol, 96.8 mg), and DIPEA (0.997 mmol, 0.174 mL) was suspended in DMF (2 mL). The mixture was stirred at ambient temperatures for 1 hour. The mixture was diluted with water and EtOAc. The aqueous phase was extracted twice with EtOAc. The combined organic phase was washed with brine, dried over MgSO₄, filtered, and concentrated in vacuo to afford Intermediate 230c. MS (m/z) 521.2 [M+H]+.

### Step 4, Preparation of (E)-3-((4-(4-(2-cyanovinyl)-2,6-dimethylphenoxy)-5,5-dimethyl-6-(1-methyl-1H-pyrazol-4-yl)-7-oxo-6,7-dihydro-5H-pyrrolo[3,4-d]pyrimidin-2-yl)amino)bicyclo[1.1.1]pentane-1-carbonitrile (Compound 230)

Intermediate 230c (0.217 mmol, 113 mg) and 2-iodoxybenzoic acid (0.434 mmol, 270 mg) was suspended in 10:1 THF/H₂O (2 mL). The mixture was stirred at 90° C. for 2 hours. The mixture was diluted with water and EtOAc. The aqueous phase was extracted twice with EtOAc. The combined organic phase was washed with brine, dried over MgSO₄, filtered, and concentrated in vacuo. The residue was redissolved in DMF and subjected to neutral phase preparative HPLC (0-100% MeCN in water) then lyophilized to afford Compound 230. (m/z) 521.3 [M+H]+. ¹H NMR (400 MHz, DMSO) δ 8.65 (s, 1H), 8.10 (s, 1H), 7.70 (s, 1H), 7.69-7.56 (m, 3H), 6.46 (d, J=16.6 Hz, 1H), 3.90 (s, 3H), 2.12 (s, 6H), 1.98 (s, 6H), 1.65 (s, 6H).

### Example 231

### (E)-3-((4-(4-(2-cyanovinyl)-2,6-dimethylphenoxy)-2-(1-methyl-1H-pyrazol-3-yl)-2H-pyrazolo[3,4-d]pyrimidin-6-yl)amino)bicyclo[1.1.1]pentane-1-carbonitrile

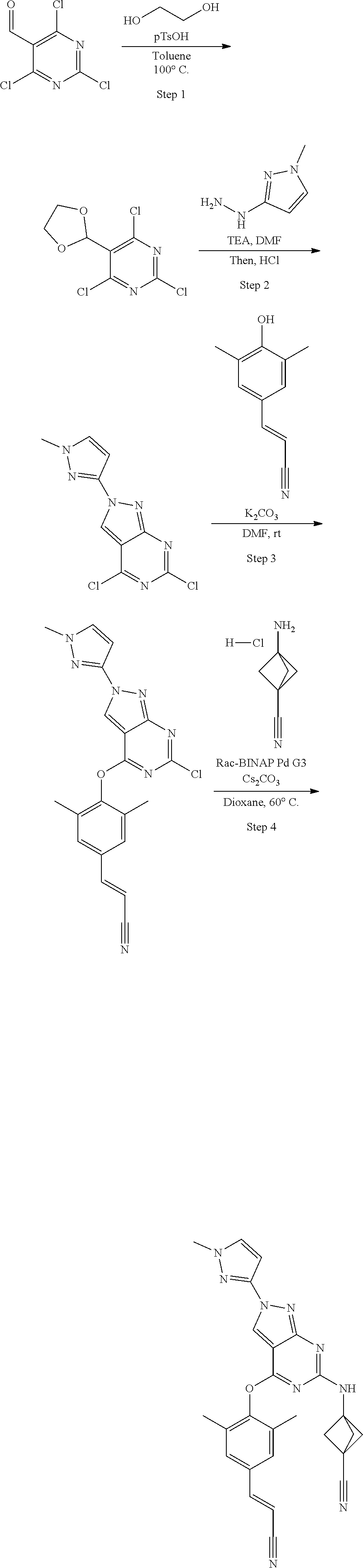

### Step 1, Preparation of 2,4,6-trichloro-5-(1,3-dioxolan-2-yl)pyrimidine (Intermediate 231a)

2,4,6-trichloropyrimidine-5-carbaldehyde (47.3 mmol, 10.0 g), 1,2-ethanediol (142 mmol, 7.91 mL), and p-toluenesulfonic acid (4.73 mmol, 900 mg) were suspended in toluene (25 mL). The mixture was stirred at 110° C. for 6 hours. The mixture was diluted with water and the resulting solids were filtered off and lyophilized to afford Intermediate 231a. MS (m/z) 254.9, 256.9 [M+H]+.

### Step 2, Preparation of 4,6-dichloro-2-(1-methyl-1H-pyrazol-3-yl)-2H-pyrazolo[3,4-d]pyrimidine (Intermediate 231b)

To a chilled solution of Intermediate 231a (2.11 mmol, 538 mg) and (1-methylpyrazol-3-yl)hydrazine; dihydrochloride (2.21 mmol, 409 mg) in DMF (8 mL) was added triethylamine (8.42 mmol, 1.17 mL) and was allowed to warm up to room temperature. The mixture was diluted with water and EtOAc. The aqueous phase was extracted twice with EtOAc. The organic phase was dried over MgSO₄, filtered, and concentrated in vacuo. The resulting residue was redissolved in DMF, treated with 4M HCl in dioxane, and stirred at room temperature for 1 hour. The mixture was quenched with aq. NaHCO₃ and water. The aqueous phase was extracted with EtOAc twice. The organic phase was dried over MgSO₄, filtered, and concentrated in vacuo to afford Intermediate 231b. MS (m/z) 269.1, 271.1 [M+H]+.

### Step 3, preparation of (E)-3-(4-((6-chloro-2-(1-methyl-1H-pyrazol-3-yl)-2H-pyrazolo[3,4-d]pyrimidin-4-yl)oxy)-3,5-dimethylphenyl)acrylonitrile (Intermediate 231c)

Intermediate 231b (2.08 mmol, 560 mg), (E)-3-(4-hydroxy-3,5-dimethyl-phenyl)prop-2-enenitrile; hydrochloride (2.08 mmol, 436 mg), and potassium carbonate (4.16 mmol, 575 mg) were suspended in DMF (8 mL). The mixture was stirred at room temperature for 1 hour and then, diluted with water. The solids were filtered and collected to yield Intermediate 231c. MS (m/z) 406.2, 408.2 [M+H]+.

### Step 4, preparation of (E)-3-((4-(4-(2-cyanovinyl)-2,6-dimethylphenoxy)-2-(1-methyl-1H-pyrazol-3-yl)-2H-pyrazolo[3,4-d]pyrimidin-6-yl)amino)bicyclo[1.1.1]pentane-1-carbonitrile (Compound 231)

Intermediate 231c (0.370 mmol, 150 mg), 3-aminobicyclo[1.1.1]pentane-1-carbonitrile (1.11 mmol, 120 mg), rac-BINAP Pd G3 (0.370 mmol, 368 mg), and cesium carbonate (1.85 mmol, 602 mg) were suspended in dioxane (4 mL). The mixture was stirred at 60° C. for 5 hours. The mixture was filtered through celite, washed with EtOAc, and concentrated in vacuo. The residue was redissolved in DMF, subjected to preparative HPLC (0-100% MeCN in water, 0.1% TFA), and lyophilized to afford the title compound as a mono-TFA salt. MS (m/z) 478.3 [M+H]+. ¹H NMR (400 MHz, Acetonitrile-d3) δ 11.37 (s, 1H), 8.93 (s, 1H), 7.67 (d, J=2.4 Hz, 1H), 7.58-7.46 (m, 3H), 6.67 (d, J=2.4 Hz, 1H), 6.14 (d, J=16.7 Hz, 1H), 3.95 (s, 3H), 2.21 (s, 6H), 2.14 (s, 6H).

### Example 232

### (E)-3-((4-(4-(2-cyanovinyl)-2,6-dimethylphenoxy)-2-(1-methyl-1H-pyrazol-4-yl)-2H-pyrazolo[3,4-d]pyrimidin-6-yl)amino)bicyclo[1.1.1]pentane-1-carbonitrile

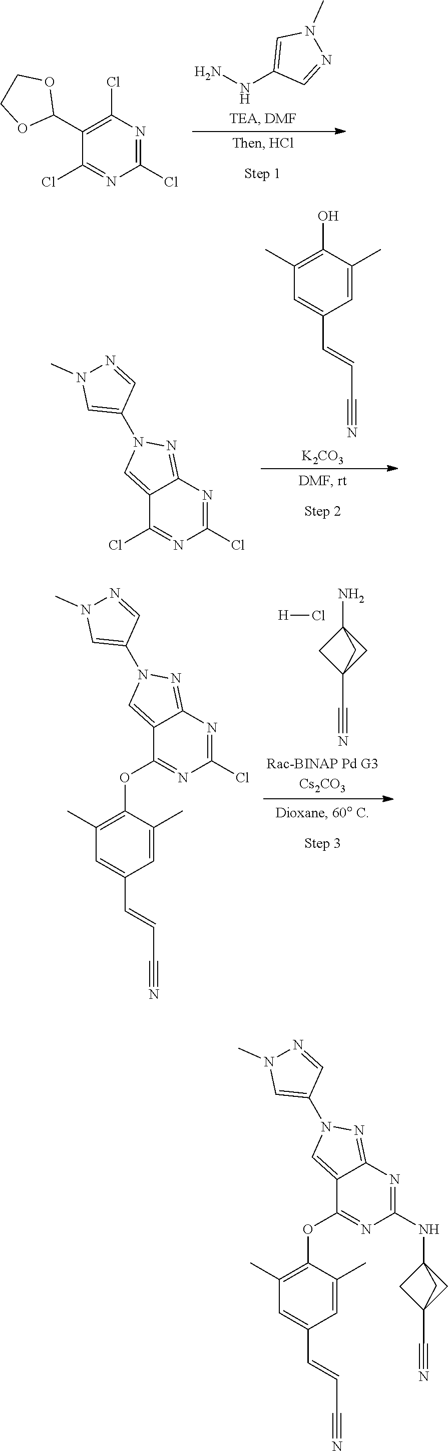

### Step 1, Preparation of 4,6-dichloro-2-(1-methylpyrazol-4-yl)pyrazolo[3,4-d]pyrimidine (Intermediate 232a)

To a chilled solution of Intermediate 231a (1.17 mmol, 300 mg) and (1-methylpyrazol-4-yl)hydrazine; dihydrochloride (1.17 mmol, 217 mg) in DMF (6 mL) was added triethylamine (5.87 mmol, 0.818 mL) and was allowed to warm up to room temperature. The mixture was diluted with water and EtOAc. The aqueous phase was extracted twice with EtOAc. The organic phase was dried over MgSO₄, filtered, and concentrated in vacuo. The resulting residue was redissolved in DMF, treated with 4M HCl in dioxane, and stirred at room temperature for 1 hour. The mixture was quenched with aq. NaHCO₃ and water. The aqueous phase was extracted with EtOAc twice. The organic phase was dried over MgSO₄, filtered, and concentrated in vacuo to afford Intermediate 232a. MS (m/z) 269.1 [M+H]+.

### Step 2, preparation of (E)-3-[4-[6-chloro-2-(1-methylpyrazol-4-yl)pyrazolo[3,4-d]pyrimidin-4-yl]oxy-3,5-dimethyl-phenyl]prop-2-enenitrile (Intermediate 232b)

Intermediate 232a (0.881 mmol, 237 mg), (E)-3-(4-hydroxy-3,5-dimethyl-phenyl)prop-2-enenitrile; hydrochloride (0.969 mmol, 203 mg), and potassium carbonate (1.76 mmol, 243 mg) were suspended in DMF (6 mL). The mixture was stirred at room temperature for 1 hour and then, diluted with water. The solids were isolated via filtration to yield Intermediate 232b. MS (m/z) 406.2 [M+H]+.

### Step 3, preparation of (E)-3-((4-(4-(2-cyanovinyl)-2,6-dimethylphenoxy)-2-(1-methyl-1H-pyrazol-4-yl)-2H-pyrazolo[3,4-d]pyrimidin-6-yl)amino)bicyclo[1.1.1]pentane-1-carbonitrile (Compound 232)

Intermediate 232b (0.246 mmol, 100 mg), 3-aminobicyclo[1.1.1]pentane-1-carbonitrile (0.739 mmol, 79.9 mg), rac-BINAP Pd G3 (0.246 mmol, 245 mg), and cesium carbonate (1.23 mmol, 401 mg) were suspended in dioxane (1 mL). The mixture was stirred at 60° C. for 4 hours. The mixture was filtered through celite, washed with EtOAc, and concentrated in vacuo. The residue was redissolved in DMF, subjected to preparative HPLC (0-100% MeCN in water, 0.1% TFA), and lyophilized to afford the title compound as a mono-TFA salt. MS (m/z) 478.3 [M+H]+. ¹H NMR (400 MHz, Acetonitrile-d3) δ 8.08 (s, 1H), 7.99 (s, 1H), 7.52 (d, J=16.7 Hz, 1H), 7.43 (s, 2H), 6.55 (s, 1H), 6.10 (d, J=16.7 Hz, 1H), 3.95 (s, 3H), 2.16 (s, 6H), 2.14 (s, 6H).

### Example 233

### (R,E)-3-((4-(4-(2-cyanovinyl)-2,6-dimethylphenoxy)-2-(tetrahydrofuran-3-yl)-2H-pyrazolo[3,4-d]pyrimidin-6-yl)amino)bicyclo[1.1.1]pentane-1-carbonitrile

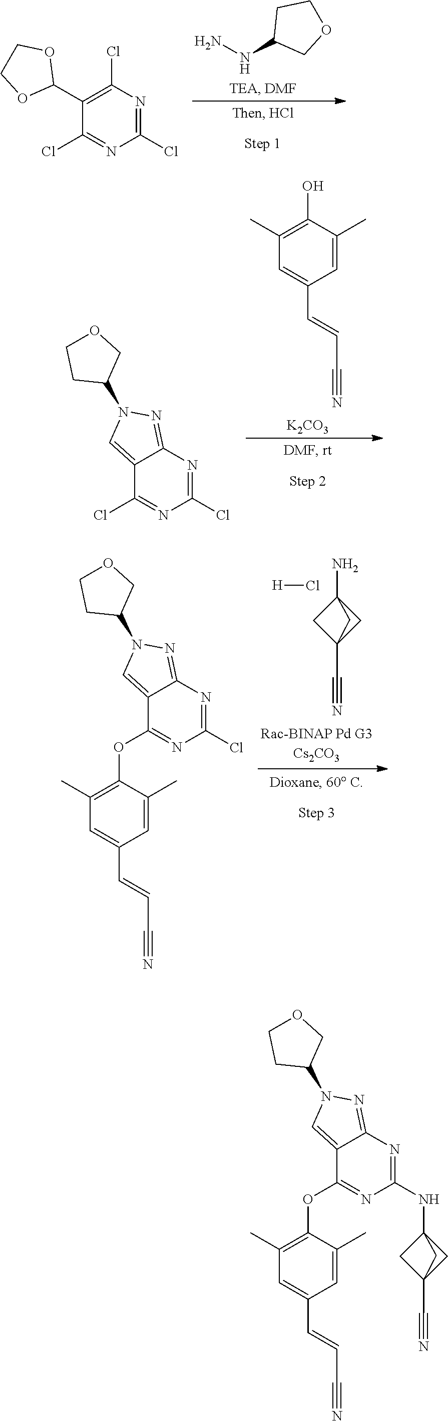

### Step 1, Preparation of 4,6-dichloro-2-[(3S)-tetrahydrofuran-3-yl]pyrazolo[3,4-d]pyrimidine (Intermediate 233a)

To a chilled solution of Intermediate 231a (1.57 mmol, 400 mg) and (R)-(tetrahydrofuran-3-yl)hydrazine; hydrochloride (1.57 mmol, 217 mg) in DMF (8 mL) was added triethylamine (6.26 mmol, 0.873 mL). The mixture instantly turned into a slurry at which point it was allowed to warm up to room temperature. The mixture was diluted with water and EtOAc. The aqueous phase was extracted twice with EtOAc. The organic phase was dried over MgSO₄, filtered, and concentrated in vacuo. The resulting residue was redissolved in DMF, treated with 4M HCl in dioxane, and stirred at room temperature for 1 hour. The mixture was quenched with aq. NaHCO₃ and water. The aqueous phase was extracted with EtOAc twice. The organic phase was dried over MgSO₄, filtered, and concentrated in vacuo to afford Intermediate 233a. MS (m/z) 259.1, 261.1 [M+H]+.

### Step 2, preparation of (E)-3-[4-[6-chloro-2-[(3S)-tetrahydrofuran-3-yl]pyrazolo[3,4-d]pyrimidin-4-yl]oxy-3,5-dimethyl-phenyl]prop-2-enenitrile (Intermediate 233b)

Intermediate 233a (1.54 mmol, 400 mg), (E)-3-(4-hydroxy-3,5-dimethyl-phenyl)prop-2-enenitrile; hydrochloride (1.54 mmol, 324 mg), and potassium carbonate (3.09 mmol, 427 mg) were suspended in DMF (6 mL). The mixture was stirred at room temperature for 1 hour and then, diluted with water. The solids were filtered to yield Intermediate 233b. MS (m/z) 396.2 [M+H]+.

### Step 3, preparation of (R,E)-3-((4-(4-(2-cyanovinyl)-2,6-dimethylphenoxy)-2-(tetrahydrofuran-3-yl)-2H-pyrazolo[3,4-d]pyrimidin-6-yl)amino)bicyclo[1.1.1]pentane-1-carbonitrile (Compound 233)

Intermediate 233b (0.126 mmol, 50 mg), 3-aminobicyclo[1.1.1]pentane-1-carbonitrile (0.379 mmol, 41 mg), rac-BINAP Pd G3 (0.126 mmol, 126 mg), and cesium carbonate (0.758 mmol, 247 mg) were suspended in dioxane (1 mL). The mixture was stirred at 60° C. for 5 hours. The mixture was filtered through celite, washed with EtOAc, and concentrated in vacuo. diluted with water and EtOAc. The residue was redissolved in DMF, purified via preparative HPLC (0-100% MeCN in water, 0.1% TFA), and lyophilized to afford the title compound as a mono-TFA salt. MS (m/z) 468.3 [M+H]+. ¹H NMR (400 MHz, Acetonitrile-d3) δ 7.83 (s, 1H), 7.51 (d, J=16.7 Hz, 1H), 7.41 (s, 2H), 6.44 (s, 1H), 6.09 (d, J=16.7 Hz, 1H), 5.40-5.29 (m, 1H), 4.17-4.06 (m, 2H), 4.03-3.88 (m, 2H), 2.47-2.37 (m, 2H), 2.14 (s, 6H).

### Example 234

### (E)-3-((4-(4-(2-cyanovinyl)-2,6-dimethylphenoxy)-2-(2,2-difluoroethyl)-2H-pyrazolo[3,4-d]pyrimidin-6-yl)amino)bicyclo[1.1.1]pentane-1-carbonitrile

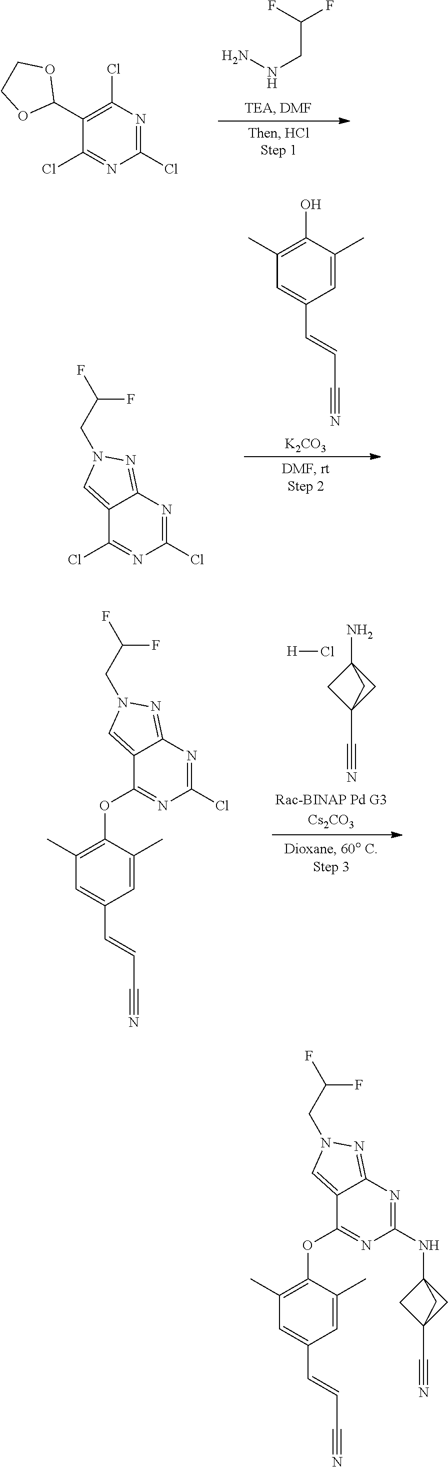

### Step 1, Preparation of 4,6-dichloro-2-(2,2-difluoroethyl)pyrazolo[3,4-d]pyrimidine (Intermediate 234a)

To a chilled solution of Intermediate 231a (1.17 mmol, 300 mg) and 2,2-difluoroethylhydrazine; hydrochloride (1.17 mmol, 156 mg) in DMF (6 mL) was added triethylamine (3.52 mmol, 0.491 mL) and was allowed to warm up to room temperature. The mixture was diluted with water and EtOAc. The aqueous phase was extracted twice with EtOAc. The organic phase was dried over MgSO₄, filtered, and concentrated in vacuo. The resulting residue was redissolved in DMF, treated with 4M HCl in dioxane, and stirred at room temperature for 1 hour. The mixture was quenched with aq. NaHCO₃ and water. The aqueous phase was extracted with EtOAc twice. The organic phase was dried over MgSO₄, filtered, and concentrated in vacuo to afford Intermediate 234a. MS (m/z) 253.1 [M+H]+.

### Step 2, preparation of (E)-3-[4-[6-chloro-2-(2,2-difluoroethyl)pyrazolo[3,4-d]pyrimidin-4-yl]oxy-3,5-dimethyl-phenyl]prop-2-enenitrile (Intermediate 234b)

Intermediate 234a (1.98 mmol, 500 mg), (E)-3-(4-hydroxy-3,5-dimethyl-phenyl)prop-2-enenitrile; hydrochloride (2.17 mmol, 456 mg), and potassium carbonate (3.95 mmol, 546 mg) were suspended in DMF (6 mL). The mixture was stirred at room temperature for 1 hour and then, diluted with water. The solids were filtered to yield Intermediate 234b. MS (m/z) 390.2 [M+H]+.

### Step 3, preparation of (E)-3-((4-(4-(2-cyanovinyl)-2,6-dimethylphenoxy)-2-(2,2-difluoroethyl)-2H-pyrazolo[3,4-d]pyrimidin-6-yl)amino)bicyclo[1.1.1]pentane-1-carbonitrile (Compound 234)

Intermediate 234b (0.257 mmol, 100 mg), 3-aminobicyclo[1.1.1]pentane-1-carbonitrile (0.770 mmol, 83.2 mg), rac-BINAP Pd G3 (0.257 mmol, 255 mg), and cesium carbonate (1.28 mmol, 418 mg) were suspended in dioxane (1 mL). The mixture was stirred at 60° C. for 5 hours. The mixture was filtered through celite, washed with EtOAc, and concentrated in vacuo. The residue was redissolved in DMF, purified via preparative HPLC (0-100% MeCN in water, 0.1% TFA), and lyophilized to afford the title compound as a mono-TFA salt. MS (m/z) 462.3 [M+H]+. ¹H NMR (400 MHz, Acetonitrile-d3) δ 7.91 (s, 1H), 7.51 (d, J=16.7 Hz, 1H), 7.42 (s, 2H), 6.47 (s, 1H), 6.29 (tt, J=55.2, 3.9 Hz, 1H), 6.10 (d, J=16.7 Hz, 1H), 4.64 (td, J=14.5, 3.8 Hz, 2H), 2.14 (s, 12H).

### Example 235

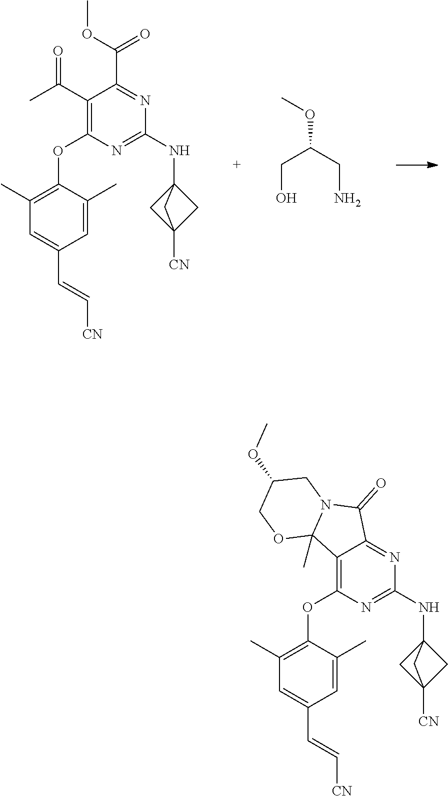

### 3-(((7R)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 235)

The reaction mixture of methyl (E)-5-acetyl-2-((3-cyanobicyclo[1.1.1]pentan-1-yl)amino)-6-(4-(2-cyanovinyl)-2,6-dimethylphenoxy)pyrimidine-4-carboxylate (Intermediate 136d) (15 mg, 0.033 mmol), (R)-3-amino-2-methoxypropan-1-ol (46 mg, 0.44 mmol) in acetonitrile (1 mL) in a sealed microwave vial was stirred at 120° C. for 1 h. Then the temperature was raised to 130° C. and the reaction mixture was heated at 130° C. overnight. The reaction was cooled down, filtered through a syringe filter and purified with reverse phase HPLC, eluting with 5-100% neutral acetonitrile and water. A single diastereomer was isolated but the stereochemistry was not confirmed. MS (m/z) 513.3 [M+H]+. ¹H NMR (400 MHz, Acetonitrile-d3) δ 7.51 (d, J=16.7 Hz, 1H), 7.43 (s, 2H), 6.78 (s, 1H), 6.10 (d, J=16.7 Hz, 1H), 4.59 (ddd, J=12.9, 5.8, 1.7 Hz, 1H), 3.95 (ddd, J=11.7, 5.0, 1.7 Hz, 1H), 3.80 (dd, J=11.7, 9.5 Hz, 1H), 3.43 (s, 3H), 3.42-3.34 (m, 1H), 3.09 (dd, J=12.9, 10.1 Hz, 1H), 2.19-2.04 (m, 12H), 1.96 (s, 3H).

### Example 236

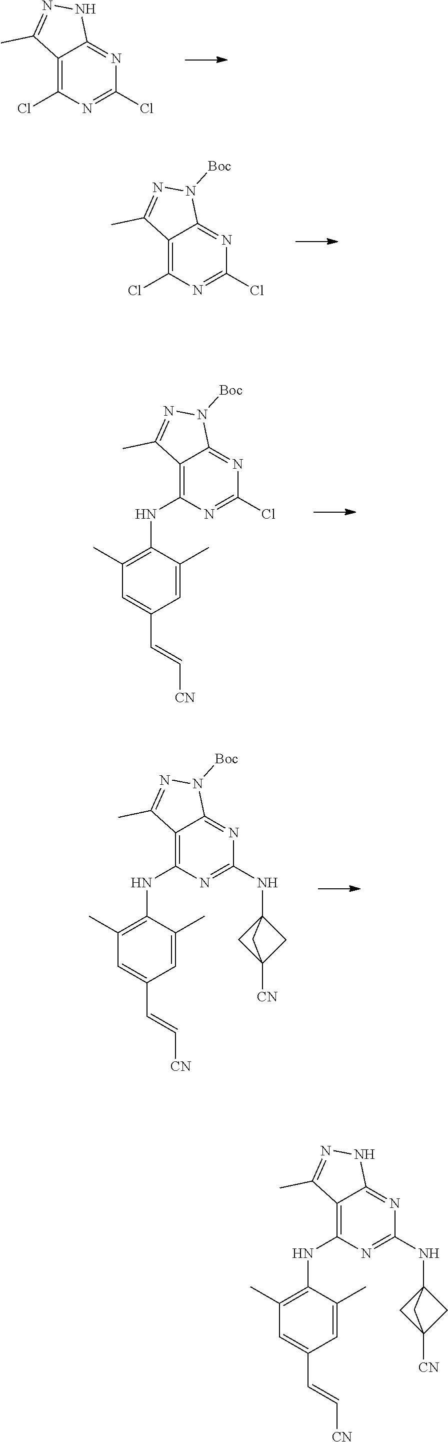 [SMILES: Cc1n[nH]c2nc(Cl)nc(Cl)c12 ; Cc1nn(C(=O)OC(C)(C)C)c2nc(Cl)nc(Cl)c12 ; Cc1cc(/C=C/C#N)cc(C)c1Nc1nc(Cl)nc2c1c(C)nn2C(=O)OC(C)(C)C ; Cc1cc(/C=C/C#N)cc(C)c1Nc1nc(NC23CC(C#N)(C2)C3)nc2c1c(C)nn2C(=O)OC(C)(C)C ; Cc1cc(/C=C/C#N)cc(C)c1Nc1nc(NC23CC(C#N)(C2)C3)nc2[nH]nc(C)c12]

### Preparation of tert-butyl 4,6-dichloro-3-methyl-pyrazolo[3,4-d]pyrimidine-1-carboxylate (Intermediate 236a)

The reaction mixture of 4,6-dichloro-3-methyl-1H-pyrazolo[3,4-d]pyrimidine (500 mg, 2.46 mmol), di-tert-butyl dicarbonate (537 mg, 2.46 mmol), N,N-diisopropylethylamine (0.429 mL, 2.46 mmol) in DCM was stirred at rt for 4 h. The reaction mixture was then directly loaded on silica gel column, eluting with 0-100% hexane/EtOAc.

### Preparation of tert-butyl 6-chloro-4-[4-[(E)-2-cyanovinyl]-2,6-dimethyl-anilino]-3-methyl-pyrazolo[3,4-d]pyrimidine-1-carboxylate (Intermediate 236b)

To a solution of tert-butyl 4,6-dichloro-3-methyl-pyrazolo[3,4-d]pyrimidine-1-carboxylate (400 mg, 1.32 mmol) in DMSO (5 mL), was added N,N-diisopropylethylamine (0.69 mL, 3.96 mmol). The solution turned green. To the mixture was added (E)-3-(4-amino-3,5-dimethyl-phenyl)prop-2-enenitrile; hydrochloride (275 mg, 1.32 mmol). The reaction mixture was stirred at rt overnight. To the reaction mixture was added water, extracted with EtOAc, dried over MgSO4, filtered, concentrated and purified by silica gel chromatography, eluting with 0-60% hexane/EtOAc.

### Preparation of tert-butyl 6-[(3-cyano-1-bicyclo[1.1.1]pentanyl)amino]-4-[4-[(E)-2-cyanovinyl]-2,6-dimethyl-anilino]-3-methyl-pyrazolo[3,4-d]pyrimidine-1-carboxylate (Intermediate 236c)

The reaction mixture of tert-butyl 6-chloro-4-[4-[(E)-2-cyanovinyl]-2,6-dimethyl-anilino]-3-methyl-pyrazolo[3,4-d]pyrimidine-1-carboxylate (80 mg, 0.182 mmol), 3-aminobicyclo[1.1.1]pentane-1-carbonitrile (59 mg, 0.55 mmol), rac-BINAP-Pd-G3 (181 mg, 0.182 mmol) and dioxane (15 mL) was charged to a round bottom flask. The mixture was degassed with argon for 10 min then stirred at 60° C. for 1 day. The reaction mixture was cooled down, washed with water, extracted with DCM, dried over MgSO4, filtered, concentrated and purified with silica gel chromatography, eluting with 0-100% hexane/EtOAc.

### Preparation of 3-[[4-[4-[(E)-2-cyanovinyl]-2,6-dimethyl-anilino]-3-methyl-11H-pyrazolo[3,4-d]pyrimidin-6-yl]amino]bicyclo[1.1.1]pentane-1-carbonitrile (Compound 236)

The reaction mixture of tert-butyl 6-[(3-cyano-1-bicyclo[1.1.1]pentanyl)amino]-4-[4-[(E)-2-cyanovinyl]-2,6-dimethyl-anilino]-3-methyl-pyrazolo[3,4-d]pyrimidine-1-carboxylate (5 mg, 0.0098 mmol) in DCM/TFA (1/1, 1 mL) was stirred at rt for 1 h. The reaction mixture was concentrated and the residue was purified by reverse phase HPLC, neutral mobile phase, eluting with 5-100% ACN in H₂O. MS (m/z) 411.2 [M+H]+. ¹H NMR (400 MHz, Methanol-d4) δ 7.54 (d, J=16.7 Hz, 1H), 7.45 (d, J=15.0 Hz, 2H), 6.25 (dd, J=16.7, 3.9 Hz, 1H), 2.62 (s, 3H), 2.28 (d, J=2.2 Hz, 6H), 2.20-2.01 (m, 6H).

### Example 237

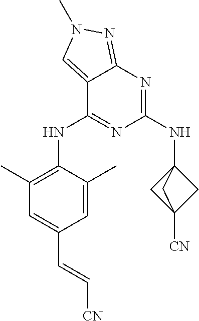 [SMILES: Cc1cc(/C=C/C#N)cc(C)c1Nc1nc(NC23CC(C#N)(C2)C3)nc2nn(C)cc12]

3-[[4-[4-[(E)-2-cyanovinyl]-2,6-dimethyl-anilino]-2-methyl-pyrazolo[3,4-d]pyrimidin-6-yl]amino]bicyclo[1.1.1]pentane-1-carbonitrile (Compound 237) was prepared in a similar manner to Compound 236, except that 4,6-dichloro-2-methyl-pyrazolo[3,4-d]pyrimidine was used instead of tert-butyl 4,6-dichloro-3-methyl-1H-pyrazolo[3,4-d]pyrimidine-1-carboxylate. MS (m/z) 411.2 [M+H]+. ¹H NMR (400 MHz, Methanol-d4) δ 8.40 (d, J=0.7 Hz, 1H), 7.58 (d, J=2.3 Hz, 1H), 7.55 (d, J=1.9 Hz, 2H), 6.31 (d, J=16.8 Hz, 1H), 4.13-4.07 (m, 3H), 2.27 (d, J=7.4 Hz, 6H), 2.13 (s, 6H).

### Examples 238, 239, and 240

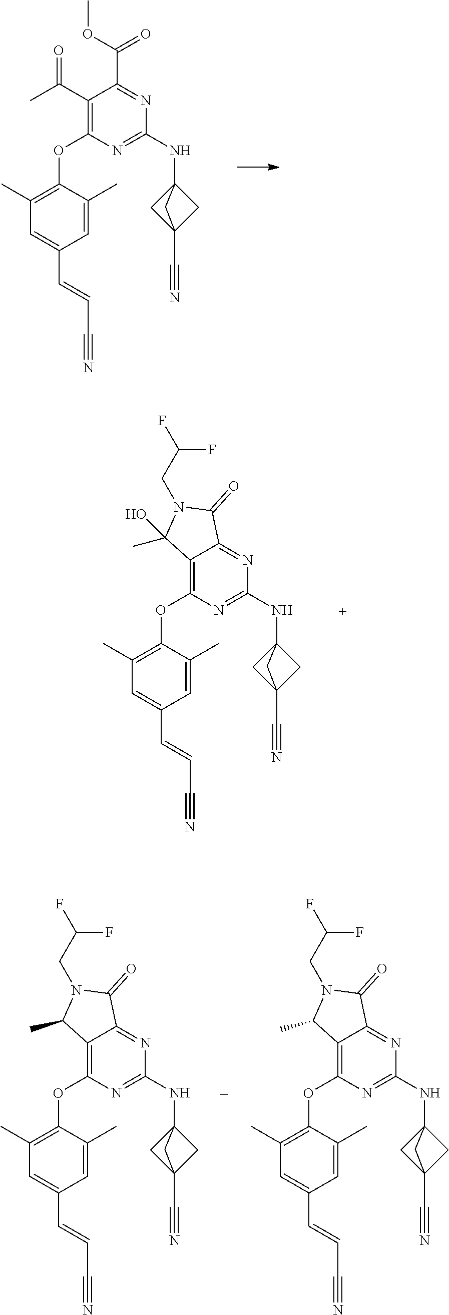 [SMILES: COC(=O)c1nc(NC23CC(C#N)(C2)C3)nc(Oc2c(C)cc(/C=C/C#N)cc2C)c1C(C)=O ; Cc1cc(/C=C/C#N)cc(C)c1Oc1nc(NC23CC(C#N)(C2)C3)nc2c1C(C)(O)N(CC(F)F)C2=O ; Cc1cc(/C=C/C#N)cc(C)c1Oc1nc(NC23CC(C#N)(C2)C3)nc2c1[C@@H](C)N(CC(F)F)C2=O ; Cc1cc(/C=C/C#N)cc(C)c1Oc1nc(NC23CC(C#N)(C2)C3)nc2c1[C@H](C)N(CC(F)F)C2=O]

(E)-3-((4-(4-(2-cyanovinyl)-2,6-dimethylphenoxy)-6-(2,2-difluoroethyl)-5-hydroxy-5-methyl-7-oxo-6,7-dihydro-5H-pyrrolo[3,4-d]pyrimidin-2-yl)amino)bicyclo[1.1.1]pentane-1-carbonitrile (Compound 238), (R,E)-3-((4-(4-(2-cyanovinyl)-2,6-dimethylphenoxy)-6-(2,2-difluoroethyl)-5-methyl-7-oxo-6,7-dihydro-5H-pyrrolo[3,4-d]pyrimidin-2-yl)amino)bicyclo[1.1.1]pentane-1-carbonitrile (Compound 239) and (S,E)-3-((4-(4-(2-cyanovinyl)-2,6-dimethylphenoxy)-6-(2,2-difluoroethyl)-5-methyl-7-oxo-6,7-dihydro-5H-pyrrolo[3,4-d]pyrimidin-2-yl)amino)bicyclo[1.1.1]pentane-1-carbonitrile (Compound 240) were prepared in a similar manner to (Compound 136), except that 2,2-difluoroethylamine was used instead of ammonium acetate. The isolated racemic material was separated using chiral SFC to afford (Compound 239) and (Compound 240). The absolute stereochemistry of the isolated enantiomers was not confirmed.

Compound 238 MS (m/z) 506.9 [M+H]+. ¹H NMR (400 MHz, Methanol-d₄) δ 7.64-7.42 (m, 2H), 6.39-6.13 (m, 2H), 4.93 (d, J=6.5 Hz, 2H), 4.31 (q, J=16.0, 15.0 Hz, 1H), 3.94-3.69 (m, 1H), 2.26-1.95 (m, 12H), 1.69 (d, J=6.6 Hz, 3H). Compound 239 MS (m/z) 491.3 [M+H]+. ¹H NMR (400 MHz, Acetonitrile-d3) δ 7.51 (d, J=16.7 Hz, 1H), 7.43 (s, 2H), 6.66 (s, 1H), 6.30-5.97 (m, 2H), 4.84 (qd, J=6.6, 3.7 Hz, 1H), 4.25 (dtd, J=17.4, 15.3, 3.5 Hz, 1H), 3.71 (tdd, J=15.1, 13.4, 4.1 Hz, 1H), 2.18-2.10 (m, 12H), 1.63 (dd, J=6.6, 3.9 Hz, 3H). Compound 240 MS (m/z) 491.3 [M+H]+. ¹H NMR (400 MHz, Acetonitrile-d3) δ 7.51 (d, J=16.7 Hz, 1H), 7.43 (s, 2H), 6.66 (s, 1H), 6.32-5.96 (m, 2H), 4.84 (q, J=6.6 Hz, 1H), 4.25 (dtd, J=17.6, 15.3, 3.5 Hz, 1H), 3.71 (tdd, J=15.2, 13.5, 4.1 Hz, 1H), 2.18-2.14 (m, 12H), 1.63 (d, J=6.6 Hz, 3H).

### Example 241

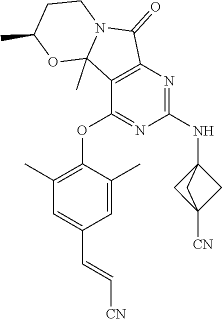 [SMILES: Cc1cc(/C=C/C#N)cc(C)c1Oc1nc(NC23CC(C#N)(C2)C3)nc2c1C1(C)O[C@@H](C)CCN1C2=O]

3-(((6S)-4-(4-((E)-2-cyanovinyl)-2,6-dimethylphenoxy)-4b,6-dimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 241) was made in similar manner to Compound 235, except that (2S)-4-aminobutan-2-ol was used instead of (R)-3-amino-2-methoxypropan-1-ol. The product was isolated as a mixture of diastereomers (˜1:1). MS (m/z) 497.3 [M+H]+. ¹H NMR (400 MHz, Acetonitrile-d3) δ 7.51 (d, J=16.7 Hz, 1H), 7.43 (s, 2H), 6.74 (s, 1H), 6.10 (d, J=16.7 Hz, 1H), 4.35-4.24 (m, 2H), 3.44-3.28 (m, 2H), 2.17-2.01 (m, 15H), 1.16 (d, J=6.1 Hz, 3H). ¹H NMR (400 MHz, Acetonitrile-d3) δ 7.51 (d, J=16.7 Hz, 1H), 7.43 (s, 2H), 6.74 (s, 1H), 6.10 (d, J=16.7 Hz, 1H), 4.35-4.24 (m, 1H), 3.86 (ddt, J=12.3, 6.1, 3.2 Hz, 1H), 2.96 (dt, J=12.4, 5.3 Hz, 1H), 2.74 (ddd, J=12.3, 9.0, 5.0 Hz, 1H), 2.17-2.01 (m, 15H), 1.10 (d, J=6.2 Hz, 3H).

### Example 242

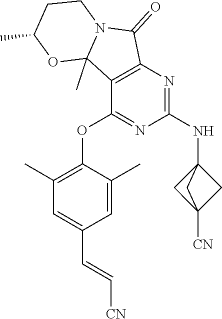 [SMILES: Cc1cc(/C=C/C#N)cc(C)c1Oc1nc(NC23CC(C#N)(C2)C3)nc2c1C1(C)O[C@H](C)CCN1C2=O]

3-(((6R)-4-(4-((E)-2-cyanovinyl)-2,6-dimethylphenoxy)-4b,6-dimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 242) was made in similar manner to Compound 235, except that (2R)-4-aminobutan-2-ol was used instead of (R)-3-amino-2-methoxypropan-1-ol. A single diastereomer was isolated but the stereochemistry was not confirmed. (m/z) 497.2 [M+H]+. ¹H NMR (400 MHz, Acetonitrile-d3) δ 7.51 (d, J=16.7 Hz, 1H), 7.43 (s, 2H), 6.73 (s, 1H), 6.10 (d, J=16.7 Hz, 1H), 4.35-4.26 (m, 2H), 3.39 (td, J=13.2, 3.6 Hz, 1H), 2.14 (m, 15H), 1.38-1.23 (m, 2H), 1.16 (d, J=6.1 Hz, 3H).

### Example 243

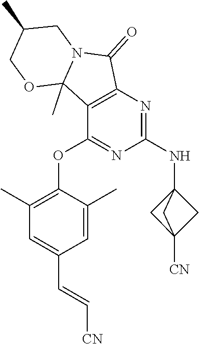 [SMILES: Cc1cc(/C=C/C#N)cc(C)c1Oc1nc(NC23CC(C#N)(C2)C3)nc2c1C1(C)OC[C@@H](C)CN1C2=O]

3-(((7S)-4-(4-((E)-2-cyanovinyl)-2,6-dimethylphenoxy)-4b,7-dimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 243) was made in similar manner to Compound 235, except that (2S)-3-amino-2-methyl-propan-1-ol was used instead of (R)-3-amino-2-methoxypropan-1-ol. A single diastereomer was isolated but the stereochemistry was not confirmed. MS (m/z) 497.2 [M+H]+. ¹H NMR (400 MHz, Acetonitrile-d3) δ 7.51 (d, J=16.7 Hz, 1H), 7.43 (s, 2H), 6.77 (bs, 1H), 6.10 (d, J=16.7 Hz, 1H), 4.29 (ddd, J=13.4, 5.0, 1.6 Hz, 1H), 3.86-3.74 (m, 2H), 2.98 (dd, J=13.4, 11.7 Hz, 1H), 2.22-2.04 (m, 12H), 1.94 (s, 3H), 1.82 (tdd, J=12.2, 10.7, 5.5 Hz, 1H), 0.93 (d, J=6.7 Hz, 3H).

### Example 244

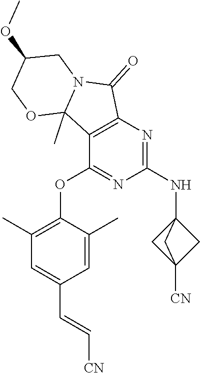 [SMILES: CO[C@@H]1COC2(C)c3c(Oc4c(C)cc(/C=C/C#N)cc4C)nc(NC45CC(C#N)(C4)C5)nc3C(=O)N2C1]

3-(((7S)-4-(4-((E)-2-cyanovinyl)-2,6-dimethylphenoxy)-7-methoxy-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 244) was made in similar manner to Compound 235, except that (2S)-3-amino-2-methoxy-propan-1-ol was used instead of (R)-3-amino-2-methoxypropan-1-ol. A single diastereomer was isolated but the stereochemistry was not confirmed. MS (m/z) 513.3 [M+H]+. ¹H NMR (400 MHz, Acetonitrile-d3) δ 7.51 (d, J=16.7 Hz, 1H), 7.43 (s, 2H), 6.77 (s, 1H), 6.10 (d, J=16.7 Hz, 1H), 4.59 (ddd, J=12.9, 5.8, 1.7 Hz, 1H), 3.96 (ddd, J=11.7, 5.0, 1.7 Hz, 1H), 3.80 (dd, J=11.7, 9.5 Hz, 1H), 3.43 (s, 3H), 3.42-3.33 (m, 1H), 3.09 (dd, J=12.9, 10.1 Hz, 1H), 2.22-2.06 (m, 15H).

### Example 245

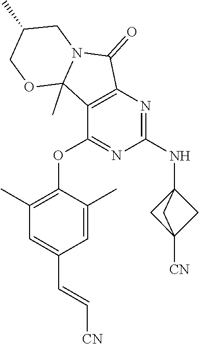 [SMILES: Cc1cc(/C=C/C#N)cc(C)c1Oc1nc(NC23CC(C#N)(C2)C3)nc2c1C1(C)OC[C@H](C)CN1C2=O]

3-(((7R)-4-(4-((E)-2-cyanovinyl)-2,6-dimethylphenoxy)-4b,7-dimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 245) was made in a similar manner to Compound 235, except that (2R)-3-amino-2-methyl-propan-1-ol hydrochloride was used instead of (R)-3-amino-2-methoxypropan-1-ol. A single diastereomer was isolated but the stereochemistry was not confirmed. MS (m/z) 497.2 [M+H]+. ¹H NMR (400 MHz, Acetonitrile-d3) δ 7.51 (d, J=16.7 Hz, 1H), 7.43 (s, 2H), 6.76 (s, 1H), 6.10 (d, J=16.7 Hz, 1H), 4.29 (ddd, J=13.4, 5.0, 1.6 Hz, 1H), 3.86-3.74 (m, 2H), 2.98 (dd, J=13.4, 11.7 Hz, 1H), 2.28-1.99 (m, 15H), 1.82 (tdd, J=12.2, 10.7, 5.4 Hz, 1H), 0.93 (d, J=6.7 Hz, 3H).

### Example 246

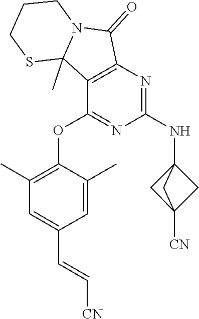 [SMILES: Cc1cc(/C=C/C#N)cc(C)c1Oc1nc(NC23CC(C#N)(C2)C3)nc2c1C1(C)SCCCN1C2=O]

(E)-3-((4-(4-(2-cyanovinyl)-2,6-dimethylphenoxy)-4b-methyl-10-oxo-4b,7,8,10-tetrahydro-6H-pyrimido[5′,4′:3,4]pyrrolo[2,1-b][1,3]thiazin-2-yl)amino)bicyclo[1.1.1]pentane-1-carbonitrile (Compound 246) was made in similar manner to Compound 235, except that 3-aminopropane-1-thiol hydrochloride and N,N-diisopropylethylamine were used instead of (R)-3-amino-2-methoxypropan-1-ol. MS (m/z) 499.4 [M+H]+. ¹H NMR (400 MHz, Acetonitrile-d3) δ 7.52 (d, J=16.7 Hz, 1H), 7.44 (s, 2H), 6.76 (bs, 1H), 6.10 (d, J=16.7 Hz, 1H), 3.38-3.18 (m, 2H), 2.78 (ddd, J=13.5, 4.1, 2.7 Hz, 2H), 2.19-2.07 (m, 15H), 1.63 (qt, J=13.2, 4.1 Hz, 2H).

### Example 247

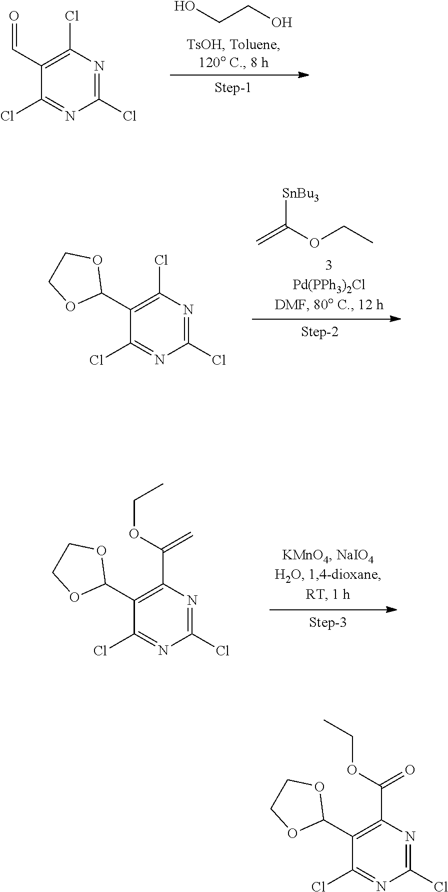

### Step 1, Preparation of 2,4,6-trichloro-5-(1,3-dioxolan-2-yl)pyrimidine (Intermediate 247a)

To a stirred solution of 2,4,6-trichloropyrimidine-5-carbaldehyde (100 g, 4.72 mmol) in toluene (10.0 vol) was added ethylene glycol (29.35 g, 4.72 mmol), p-toluene sulphonic acid monohydrate (17.99 g, 9.45 mmol) and the reaction was stirred for 8 hours at 120° C. The progress of the reaction was monitored by TLC and LCMS. After completion, the reaction was cooled to RT, concentrated under reduced pressure at <48° C. and dried to obtain the crude compound as a yellow solid. The crude compound was purified by silica gel column chromatography (60-120 mesh size) and the column was gradually eluted with 20-30% ethyl acetate/pet ether to give desired product (90 g, 74%) as a white solid.

### Step 2, Preparation of 2,4-dichloro-5-(1,3-dioxolan-2-yl)-6-(1-ethoxyvinyl)pyrimidine (Intermediate 247b)

To a stirred solution of 2,4,6-trichloro-5-(1,3-dioxolan-2-yl)pyrimidine (85 g, 3.346 mmol) and Pd(PPh₃)₂Cl₂ (11.74 g, 1.672 mmol) in dimethylformamide (10.0 vol.) under nitrogen atmosphere was added tributyl(1-ethoxyvinyl)stannane (120.85 g, 3.346 mmol) dropwise. The mixture was heated to 80° C. for 12 hours. The progress of the reaction was monitored by TLC and LCMS. After completion of the reaction, mass was cooled to 5-10° C. and poured into a solution potassium fluoride (8.5 g; in 430 mL of water) solution. Compound was extracted from ethyl acetate (2×50 ml) and organic layer was washed with water (1 lit.). The organic layer was concentrated under residue pressure to get crude compound. Crude compound was purified by silica gel column chromatography (100-200 size mesh size) and column was gradually eluted with 10-15% ethyl acetate/pet ether to give desired product (60 g, 62%) as a pale yellow oil.

### Step 3, preparation of ethyl 2,6-dichloro-5-(1,3-dioxolan-2-yl)pyrimidine-4-carboxylate (Intermediate 247c)

The suspension of sodium metaperiodate (84.24 g, 3.938 mmol) in water (850 ml) was sonicated until a clear solution was obtained. This solution was added to a stirred solution of 2,4-dichloro-5-(1,3-dioxolan-2-yl)-6-(1-ethoxyvinyl)pyrimidine (50 g, 1.712 mmol) in 1,4-dioxane (500 mL) and followed by added potassium permanganate (5.41 g, 3.423 mmol). The reaction mass was stirred at room temperature for 2 hours. The progress of the reaction was monitored by TLC and LCMS. After the reaction completion, the mass was filtered through celite bed, filtrate was diluted with saturated solution of sodium bicarbonate and sodium chloride. Compound was extracted with ethyl acetate (2×500 ml). The organic layer was dried over anhydrous sodium sulphate and concentrated under reduced pressure to get crude compound. Crude was purified by silica gel column chromatography (230-400 mesh size) and column was gradually eluted with 10-15% ethyl acetate/pet ether and followed by slurry wash with 3 vol of 80% aq. Methanol to get as a white solid (25 g, 50%).

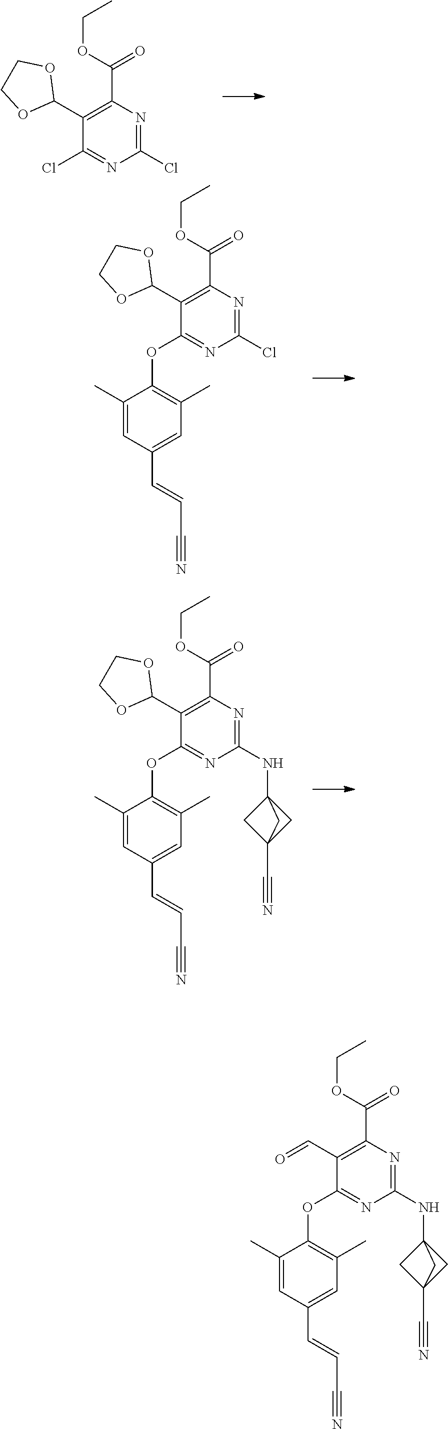

### Preparation of ethyl (E)-2-((3-cyanobicyclo[1.1.1]pentan-1-yl)amino)-6-(4-(2-cyanovinyl)-2,6-dimethylphenoxy)-5-formylpyrimidine-4-carboxylate (Intermediate 247d)

To a solution of ethyl 2,6-dichloro-5-(1,3-dioxolan-2-yl)pyrimidine-4-carboxylate (Intermediate 247c) (10.0 g, 34.1 mmol) in NMP (150 mL) was added potassium carbonate (7.07 g, 51.2 mmol) and (E)-3-(4-hydroxy-3,5-dimethyl-phenyl)prop-2-enenitrile (6.21 g, 35.8 mmol). The reaction mixture was stirred at rt for 1 h. Then to the mixture was added potassium carbonate (17.3 g, 0.125 mol) and 3-aminobicyclo[1.1.1]pentane-1-carbonitrile; hydrochloride (12.9 g, 0.0895 mol). The reaction mixture was stirred at 60° C. overnight. After the reaction was cooled down, half of the reaction mixture was treated with 100 mL 1N HCl and sonicated at rt for 1 h. 200 mL THE was added to dissolve most of the solid then stirred at rt for 1 h. The reaction mixture was then added to 50 mL brine and extracted with EtOAc. The combined organic layer was washed with NaHCO₃ and dried over Na₂SO₄, filtered and concentrated.

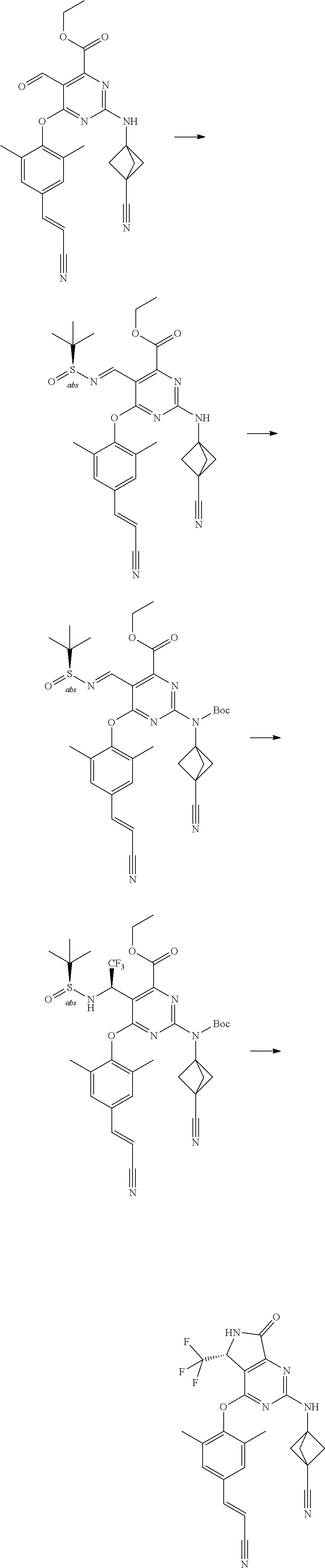

### Preparation of ethyl 5-((E)-(((S)-tert-butylsulfinyl)imino)methyl)-2-((3-cyanobicyclo[1.1.1]pentan-1-yl)amino)-6-(4-((E)-2-cyanovinyl)-2,6-dim ethylphenoxy)pyrimidine-4-carboxylate (Intermediate 247e)

The reaction mixture of ethyl (E)-2-((3-cyanobicyclo[1.1.1]pentan-1-yl)amino)-6-(4-(2-cyanovinyl)-2,6-dimethylphenoxy)-5-formylpyrimidine-4-carboxylate (Intermediate 247d) (136 mg, 0.3 mmol), cupric sulfate (95 mg, 0.6 mmol) and (S)-(−)-2-methyl-2-propanesulfinamide (40 mg, 0.33 mmol) was stirred at 55° C. overnight. The reaction mixture was cooled down, to the mixture was added water, extracted with EtOAc. The organic phase was dried over MgSO4, filtered, concentrated and purified using silica gel chromatography, eluting with 0-100% hexane/EtOAc.

### Preparation of ethyl 2-((tert-butoxycarbonyl)(3-cyanobicyclo[1.1.1]pentan-1-yl)amino)-5-((E)-(((S)-tert-butylsulfinyl)imino)methyl)-6-(4-((E)-2-cyanovinyl)-2,6-dimethylphenoxy)pyrimidine-4-carboxylate (Intermediate 247f)

The reaction mixture of ethyl 5-((E)-(((S)-tert-butylsulfinyl)imino)methyl)-2-((3-cyanobicyclo[1.1.1]pentan-1-yl)amino)-6-(4-((E)-2-cyanovinyl)-2, 6-dimethylphenoxy)pyrimidine-4-carboxylate (Intermediate 247e) (30 mg, 0.054 mmol), triethylamine (37 uL, 0.29 mmol), di-tert-butyl dicarbonate (23.4 mg, 0.107 mmol) and 4-dimethylaminopyridine (1.96 mg, 0.01 mmol) in DCM was stirred at rt for 1 h. The reaction mixture was directly loaded on silica gel column, purified using silica gel chromatography, eluting with 0-100% hexane/EtOAc.

### Preparation of ethyl 2-((tert-butoxycarbonyl)(3-cyanobicyclo[1.1.1]pentan-1-yl)amino)-5-((R)-1-(((S)-tert-butylsulfinyl)amino)-2,2,2-trifluoroethyl)-6-(4-((E)-2-cyanovinyl)-2,6-dimethylphenoxy)pyrimidine-4-carboxylate (Intermediate 247g)

To a solution of ethyl 2-[tert-butoxycarbonyl-(3-cyano-1-bicyclo[1.1.1]pentanyl)amino]-5-[(E)-tert-butylsulfinyliminomethyl]-6-[4-[(E)-2-cyanovinyl]-2,6-dimethyl-phenoxy]pyrimidine-4-carboxylate (Intermediate 247f) (17 mg, 0.026 mmol) in THE (2 mL) was added tetrabutylammonium difluorotriphenylsilicate (41.2 mg, 0.077 mmol) in THE (1 mL), 1 mL of (Trifluoromethyl)trimethylsilane stock solution (70 mg in 2 mL) at −78° C. The reaction mixture was stirred at −78° C. for 0.5 h. Then allowed temperature to raise to −20° C. The reaction mixture was stirred at −20° C. for 45 min. To the mixture was added sat. NH₄Cl solution, warmed up to rt, extracted with EtOAc. The organic phase was dried over MgSO₄, filtered, concentrated and purified with silica gel chromatography, eluting with 0-80% hexane/EtOAc.

### Preparation of (R,E)-3-((4-(4-(2-cyanovinyl)-2,6-dimethylphenoxy)-7-oxo-5-(trifluoromethyl)-6,7-dihydro-5H-pyrrolo[3,4-d]pyrimidin-2-yl)amino)bicyclo[1.1.1]pentane-1-carbonitrile (Compound 247)

To ethyl 2-((tert-butoxycarbonyl)(3-cyanobicyclo[1.1.1]pentan-1-yl)amino)-5-((R)-1-(((S)-tert-butylsulfinyl)amino)-2,2,2-trifluoroethyl)-6-(4-((E)-2-cyanovinyl)-2,6-dimethylphenoxy)pyrimidine-4-carboxylate (Intermediate 247g) (20 mg, 0.027 mmol) in MeOH (3 mL) was added 5 drops of concentrated HCl solution. The reaction mixture was stirred at rt overnight. Then the mixture was stirred at 40° C. for 5 h. The reaction mixture was concentrated and the residue was purified with reverse phase HPLC, eluting with 5-100% ACN/H₂O, containing 0.1% TFA. MS (m/z) 481.2 [M+H]+. ¹H NMR (400 MHz, Acetonitrile-d3) δ 7.84 (s, 1H), 7.51 (d, J=16.7 Hz, 1H), 7.43 (s, 2H), 6.92 (bs, 1H), 6.10 (d, J=16.7 Hz, 1H), 5.42-5.34 (m, 1H), 2.18-1.99 (m, 12H). ¹⁹F NMR (376 MHz, Acetonitrile-d3) δ −75.23 (d, J=5.7 Hz).

### Examples 248 and 249

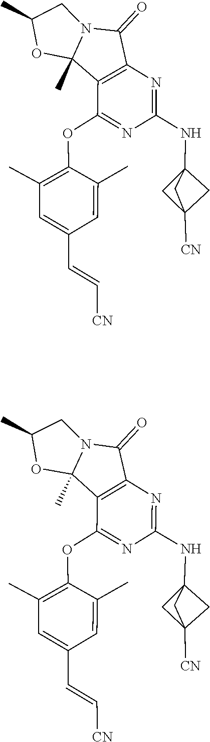 [SMILES: Cc1cc(/C=C/C#N)cc(C)c1Oc1nc(NC23CC(C#N)(C2)C3)nc2c1[C@]1(C)O[C@@H](C)CN1C2=O ; Cc1cc(/C=C/C#N)cc(C)c1Oc1nc(NC23CC(C#N)(C2)C3)nc2c1[C@@]1(C)O[C@@H](C)CN1C2=O]

3-(((4bS,6S)-4-(4-((E)-2-cyanovinyl)-2,6-dimethylphenoxy)-4b,6-dimethyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 248) and 3-(((4bR,6S)-4-(4-((E)-2-cyanovinyl)-2,6-dimethylphenoxy)-4b,6-dimethyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 249) were made in a similar manner to Compound 235, except that (2S)-1-aminopropan-2-ol was used instead of (R)-3-amino-2-methoxypropan-1-ol and the reaction mixture was heated at 150° C. The relative stereochemistry was assigned arbitrarily and not confirmed.

For nonpolar diastereomer Compound 248: MS (m/z) 483. [M+H]+. ¹H NMR (400 MHz, Acetonitrile-d3) δ 7.51 (d, J=16.7 Hz, 1H), 7.44 (d, J=3.5 Hz, 2H), 6.77 (s, 2H), 6.10 (d, J=16.7 Hz, 1H), 4.27-4.10 (m, 3H), 3.08 (dd, J=11.4, 9.2 Hz, 1H), 2.15 (d, J=7.2 Hz, 12H), 1.88 (d, J=6.6 Hz, 3H), 1.35 (d, J=5.9 Hz, 3H). For polar diastereomer Compound 249: MS (m/z) 483.2 [M+H]+. ¹H NMR (400 MHz, Acetonitrile-d3) δ 7.51 (d, J=16.7 Hz, 1H), 7.43 (s, 2H), 6.78 (s, 1H), 6.10 (d, J=16.7 Hz, 1H), 4.85-4.72 (m, 1H), 3.65 (dd, J=11.3, 6.9 Hz, 1H), 3.52 (dd, J=11.3, 5.4 Hz, 1H), 2.15-2.00 (m, 12H), 1.88 (d, J=6.7 Hz, 3H), 1.20 (d, J=6.2 Hz, 3H).

### Example 250

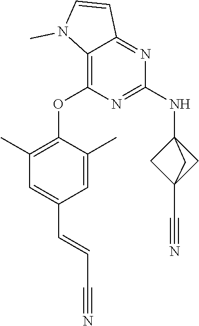 [SMILES: Cc1cc(/C=C/C#N)cc(C)c1Oc1nc(NC23CC(C#N)(C2)C3)nc2ccn(C)c12]

### Preparation of 3-[[4-[4-[(E)-2-cyanovinyl]-2,6-dimethyl-phenoxy]-5-methyl-pyrrolo[3,2-d]pyrimidin-2-yl]amino]bicyclo[1.1.1]pentane-1-carbonitrile (Compound 250)

To a solution of 3-[[4-[4-[(E)-2-cyanovinyl]-2,6-dimethyl-phenoxy]-5H-pyrrolo[3,2-d]pyrimidin-2-yl]amino]bicyclo[1.1.1]pentane-1-carbonitrile (24 mg, 0.061 mmol) (Compound 115) in DMF (2 mL), was added potassium carbonate (41.8 mg, 0.303 mmol) and iodomethane in tert-butyl methyl ether (2.0 mol/L, 0.03 mL). The reaction mixture was stirred at rt for one day then sodium hydride (4.2 mg, 0.18 mmol) was added. The reaction mixture was stirred at rt for 0.5 h. The reaction mixture was filtered, purified with reverse phase HPLC, eluting with 5-100% neutral ACN/water. MS (m/z) 411.2 [M+H]+. ¹H NMR (400 MHz, Acetonitrile-d3) δ 10.45 (s, 1H), 7.58-7.48 (m, 2H), 7.47 (s, 2H), 6.30 (d, J=2.9 Hz, 1H), 6.13 (d, J=16.7 Hz, 1H), 4.12 (s, 3H), 2.21 (s, 6H), 2.08 (s, 6H).

### Example 251

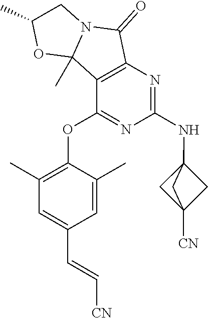 [SMILES: Cc1cc(/C=C/C#N)cc(C)c1Oc1nc(NC23CC(C#N)(C2)C3)nc2c1C1(C)O[C@H](C)CN1C2=O]

3-(((6R)-4-(4-((E)-2-cyanovinyl)-2,6-dimethylphenoxy)-4b,6-dimethyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 251) was made in a similar manner to Compound 235, except that (2R)-1-aminopropan-2-ol was used instead of (R)-3-amino-2-methoxypropan-1-ol and the reaction mixture was heated at 150° C. The less polar product was isolated as a single diastereomer but the stereochemistry was not confirmed. (m/z) 483.3 [M+H]+. ¹H NMR (400 MHz, Acetonitrile-d3) δ 7.51 (d, J=16.7 Hz, 1H), 7.44 (d, J=3.4 Hz, 2H), 6.76 (s, 1H), 6.10 (d, J=16.8 Hz, 1H), 4.23 (dd, J=11.5, 5.6 Hz, 1H), 4.15 (dt, J=9.2, 5.7 Hz, 1H), 3.08 (dd, J=11.4, 9.2 Hz, 1H), 2.16 (s, 12H), 1.89 (s, 3H), 1.35 (d, J=5.9 Hz, 3H).

### Example 252

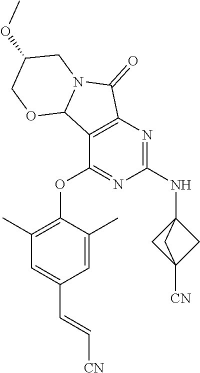 [SMILES: CO[C@H]1COC2c3c(Oc4c(C)cc(/C=C/C#N)cc4C)nc(NC45CC(C#N)(C4)C5)nc3C(=O)N2C1]

3-(((7R)-4-(4-((E)-2-cyanovinyl)-2,6-dimethylphenoxy)-7-methoxy-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 252) was prepared in a similar manner to Compound 235, except that (2R)-1-aminopropan-2-ol was used instead of (R)-3-amino-2-methoxypropan-1-ol and the reaction mixture was heated at 120° C. A single diastereomer was isolated but the stereochemistry was not confirmed. MS (m/z) 499.3 [M+H]+. ¹H NMR (400 MHz, Acetonitrile-d3) δ 7.51 (d, J=16.7 Hz, 1H), 7.42 (s, 2H), 6.81 (s, 1H), 6.09 (d, J=16.7 Hz, 1H), 5.79 (s, 1H), 4.64 (ddd, J=12.6, 5.5, 1.9 Hz, 1H), 4.28 (ddd, J=11.1, 4.7, 1.8 Hz, 1H), 3.63 (t, J=10.7 Hz, 1H), 3.42 (s, 3H), 3.01 (dd, J=12.6, 10.1 Hz, 1H), 2.04-2.16 (m, 12H).

### Example 253

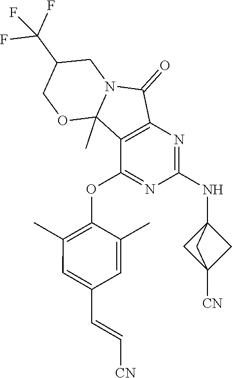 [SMILES: Cc1cc(/C=C/C#N)cc(C)c1Oc1nc(NC23CC(C#N)(C2)C3)nc2c1C1(C)OCC(C(F)(F)F)CN1C2=O]

(E)-3-((4-(4-(2-cyanovinyl)-2,6-dimethylphenoxy)-4b-methyl-10-oxo-7-(trifluoromethyl)-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 253) was prepared made in a similar manner to Compound 235, except that 2-(aminomethyl)-3,3,3-trifluoro-propan-1-ol was used instead of (R)-3-amino-2-methoxypropan-1-ol and the reaction mixture was heated at 120° C. The product was isolated as a single pair of diastereomers but the stereochemistry was not confirmed. MS (m/z) 551.3 [M+H]+. ¹H NMR (400 MHz, Acetonitrile-d3) δ 7.51 (d, J=16.7 Hz, 1H), 7.43 (s, 2H), 6.83 (s, 1H), 6.10 (d, J=16.7 Hz, 1H), 4.53 (ddd, J=13.5, 5.6, 1.6 Hz, 1H), 4.20 (dd, J=12.2, 10.3 Hz, 1H), 4.06 (ddd, J=12.1, 4.9, 1.6 Hz, 1H), 3.48 (dd, J=13.5, 11.5 Hz, 1H), 2.88-2.72 (m, 1H), 2.18-2.03 (m, 12H), 2.16 (s, 3H).

### Example 254

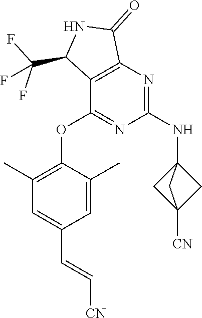 [SMILES: Cc1cc(/C=C/C#N)cc(C)c1Oc1nc(NC23CC(C#N)(C2)C3)nc2c1[C@@H](C(F)(F)F)NC2=O]

(S,E)-3-((4-(4-(2-cyanovinyl)-2,6-dimethylphenoxy)-7-oxo-5-(trifluoromethyl)-6,7-dihydro-5H-pyrrolo[3,4-d]pyrimidin-2-yl)amino)bicyclo[1.1.1]pentane-1-carbonitrile (Compound 254) was made in a similar manner to Compound 247, except that (R)-(+)-2-methyl-2-propanesulfinamide was used instead of (S)-(−)-2-methyl-2-propanesulfinamide. MS (m/z) 481.2 [M+H]+. ¹H NMR (400 MHz, Acetonitrile-d3) δ 7.80 (s, 1H), 7.51 (d, J=16.7 Hz, 1H), 7.43 (s, 2H), 6.90 (s, 1H), 6.10 (d, J=16.7 Hz, 1H), 5.39 (tt, J=6.2, 3.1 Hz, 1H), 2.12 (s, 12H). ¹⁹F NMR (376 MHz, Acetonitrile-d3) δ −77.28.

### Example 255

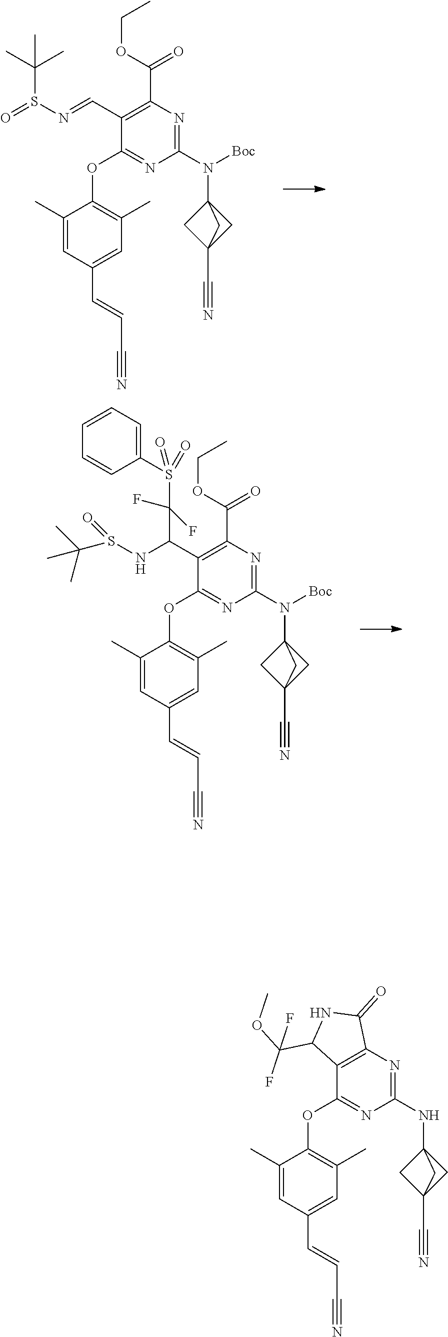

### Preparation of ethyl (E)-2-((tert-butoxycarbonyl)(3-cyanobicyclo[1.1.1]pentan-1-yl)amino)-5-(1-((tert-butylsulfinyl)amino)-2,2-difluoro-2-(phenylsulfonyl)ethyl)-6-(4-(2-cyanovinyl)-2,6-dimethylphenoxy)pyrimidine-4-carboxylate (Intermediate 255a)

To a solution of ethyl 2-[tert-butoxycarbonyl-(3-cyano-1-bicyclo[1.1.1]pentanyl)amino]-5-[(E)-tert-butylsulfinyliminomethyl]-6-[4-[(E)-2-cyanovinyl]-2,6-dimethyl-phenoxy]pyrimidine-4-carboxylate (60 mg, 0.091 mmol) in THF (2 mL) was added LiHMNDS (1M in THF, 272 μL, 0.27 mmol) and added difluoromethylsulfonylbenzene (26.2 mg, 0.136 mmol) at −78° C. Then the temperature was allowed to slowly warm up to −20° C. After the reaction finished, to the mixture was added water, extracted with EtOAc, the organic was separated, dried over MgSO4, filtered, concentrated and purified by silica gel chromatography, 0-100% hexane/EtOAc. The collected product was purified again with reverse phase HPLC, eluting with 5-100% acid modified ACN/H₂O.

### Preparation of (E)-3-((4-2-((2-cyanovinyl)-2,6-dimethylphenoxy)-5-(difluoro(methoxy)methyl)-7-oxo-6,7-dihydro-5H-pyrrolo[3,4-d]pyrimidin-2-yl)amino)bicyclo[1.1.1]pentane-1-carbonitrile (Compound 255)

To a solution of ethyl 5-[2-(benzenesulfonyl)-1-(tert-butylsulfinylamino)-2,2-difluoroethyl]-2-[tert-butoxycarbonyl-(3-cyano-1-bicyclo[1.1.1]pentanyl)amino]-6-[4-[(E)-2-cyanovinyl]-2,6-dimethyl-phenoxy]pyrimidine-4-carboxylate (6.5 mg, 0.0076 mmol) in MeOH (1 mL) was added 2 drops of concentrated HCl. The reaction mixture was heated at 40° C. overnight. The reaction mixture was washed with NaHCO₃, extracted with DCM, the organic phase was separated, dried over MgSO₄, filtered, concentrated and purified by silica gel chromatography, eluting with 0-80% hexane/EtOAc. MS (m/z) 493.2 [M+H]+. ¹H NMR (400 MHz, Acetonitrile-d3) δ 7.62 (s, 1H), 7.51 (d, J=16.7 Hz, 1H), 7.43 (d, J=3.7 Hz, 2H), 6.79 (s, 1H), 6.10 (d, J=16.7 Hz, 1H), 5.15 (td, J=5.1, 1.4 Hz, 1H), 3.63 (s, 3H), 2.19-2.01 (m, 12H).

### Example 256

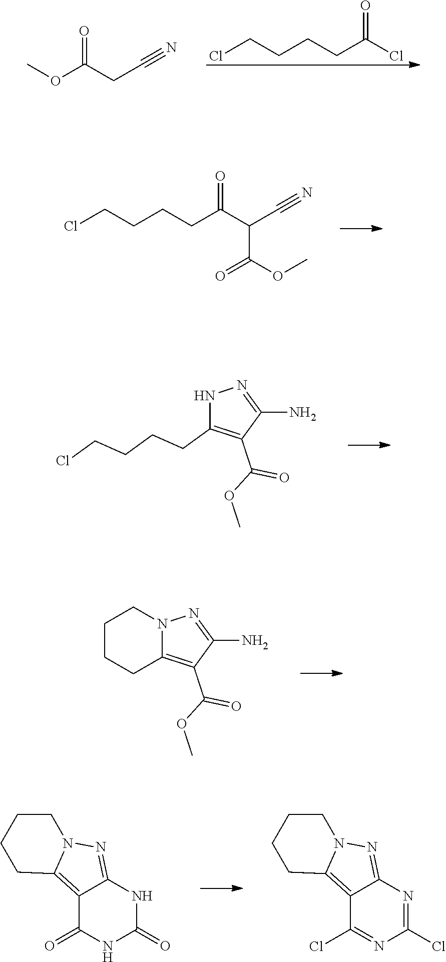

### Preparation of methyl 7-chloro-2-cyano-3-oxoheptanoate (Intermediate 256a)

To a stirred solution of methyl cyanoacetate (150 g, 1.51 mol, 1.00 eq.) in THE (1.50 L) was added Et₃N (153 g, 1.51 mol, 1.00 eq.) at 0° C. To the above mixture was added 5-chloropentanoyl chloride (328 g, 2.12 mol, 1.40 eq.) in THF (500 mL) at 0° C. The resulting mixture was stirred at room temperature for 3 h. The resulting mixture was concentrated under reduced pressure. The residue was purified by silica gel column chromatography, eluted with PE/EA (10:1) to afford methyl 7-chloro-2-cyano-3-oxoheptanoate (260 g, 78.9% yield, 80% purity) as yellow oil. LCMS (ESI, m z): 216.0 [M−H]⁻.

### Preparation of methyl 3-amino-5-(4-chlorobutyl)-1H-pyrazole-4-carboxylate (Intermediate 256b)

To a stirred solution of methyl 7-chloro-2-cyano-3-oxoheptanoate (240 g, 1.10 mol, 1.00 eq.) in MeCN (2.40 L) was added hydrazine monohydrate (55.2 g, 1.10 mmol, 1.00 eq.) at room temperature. The resulting mixture was stirred at room temperature overnight. The resulting mixture was concentrated under reduced pressure. The residue was purified by prep-HPLC with the following conditions [column: C18; mobile phase A: Water (0.1% FA), mobile phase B: MeCN; Flow rate: 60 mL/min; Gradient: 0% B to 100% B in 20 min; Wave Length: 254 nm/220 nm] to afford methyl 3-amino-5-(4-chlorobutyl)-1H-pyrazole-4-carboxylate (47.0 g, 18.4% yield, 95% purity) as a yellow oil. LCMS (ESI, m z): 232.1 [M+H]+

### Preparation of methyl 2-amino-4,5,6,7-tetrahydropyrazolo[1,5-a]pyridine-3-carboxylate (Intermediate 256c)

To a stirred solution of methyl 3-amino-5-(4-chlorobutyl)-1H-pyrazole-4-carboxylate (46.0 g, 198 mmol, 1.00 eq.) in DMSO (450 mL) was added KOH (126 g, 2.25 mmol, 11.3 eq.) at room temperature. The resulting mixture was stirred at room temperature overnight. The resulting mixture was diluted with water (2.0 L). The resulting mixture was extracted with DCM (3×1.0 L). The combined organic layers were washed with brine (1×1.0 L), dried over anhydrous Na₂SO₄. After filtration, the filtrate was concentrated under reduced pressure. The residue was purified by Prep-HPLC with the following conditions [mobile phase A: Water (0.1% FA), mobile phase B: MeCN; Flow rate: 60 mL/min; Gradient: 0% B to 100% B in 30 min; Wave Length: 254 nm/220 nm] to afford methyl 2-amino-4,5,6,7-tetrahydropyrazolo[1,5-a]pyridine-3-carboxylate (18.6 g, 48.0% yield, 95.0% purity) as yellow oil. LCMS (ESI, m z): 196.1 [M+H]⁺.

### Preparation of 5,6,7,8-tetrahydropyrido[1′,2′:1,5]pyrazolo[3,4-d]pyrimidine-2,4(1H,3H)-dione (Intermediate 256d)

A mixture of methyl 2-amino-4,5,6,7-tetrahydropyrazolo[1,5-a]pyridine-3-carboxylate (18.6 g, 95.2 mmol, 1.00 equiv) and urea (28.6 g, 476 mmol, 5.00 equiv) was stirred at 160° C. for 3 h. The mixture was allowed to cool down to room temperature. The resulting mixture was diluted with water (100 mL) and stirred at 0° C. for 0.5 h. The precipitated solids were collected by filtration and washed with water (3×10 mL). This resulted in 5,6,7,8-tetrahydropyrido[1′,2′:1,5]pyrazolo[3,4-d]pyrimidine-2,4(1H,3H)-dione (12.0 g, crude) as white solid. LCMS (ESI, m z): 207.0 [M+H]⁺.

### Preparation of 2,4-dichloro-5,6,7,8-tetrahydropyrido[1′,2′:1,5]pyrazolo[3,4-d]pyrimidine (Intermediate 256e)

A mixture of 5,6,7,8-tetrahydropyrido[1′,2′:1,5]pyrazolo[3,4-d]pyrimidine-2,4(1H,3H)-dione (10.5 g, 50.9 mmol, 1.00 equiv) and phosphoroyl trichloride (100 mL) was stirred at 120° C. for 8 h. The resulting mixture was concentrated under reduced pressure. The residue was dissolved in DCM (100 mL). The reaction was quenched by pouring the mixture to sat. NaHCO₃ (aq.) at 0° C. The resulting mixture was extracted with DCM (3×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (1:9) to afford 2,4-dichloro-5,6,7,8-tetrahydropyrido[1′,2′:1,5]pyrazolo[3,4-d]pyrimidine (5.1611 g, 41.7% yield, 95.5% purity) as a white solid. LCMS (ESI, m z): 243.0 [M+H]⁺.

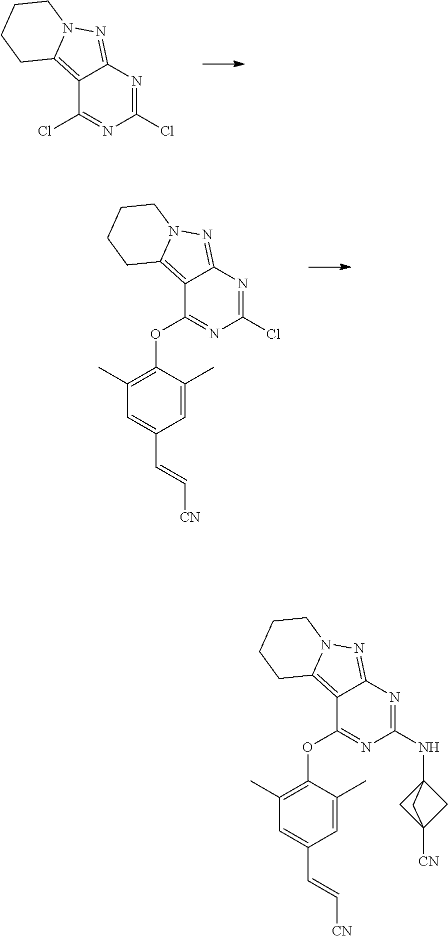 [SMILES: Clc1nc(Cl)c2c3n(nc2n1)CCCC3 ; Cc1cc(/C=C/C#N)cc(C)c1Oc1nc(Cl)nc2nn3c(c12)CCCC3 ; Cc1cc(/C=C/C#N)cc(C)c1Oc1nc(NC23CC(C#N)(C2)C3)nc2nn3c(c12)CCCC3]

### Preparation of (E)-3-(4-((2-chloro-5,6,7,8-tetrahydropyrido[1′,2′:1,5]pyrazolo[3,4-d]pyrimidin-4-yl)oxy)-3,5-dimethylphenyl)acrylonitrile (Intermediate 256f)

The reaction mixture of 2,4-dichloro-5,6,7,8-tetrahydropyrido[1′,2′:1,5]pyrazolo[3,4-d]pyrimidine (500 mg, 2.18 mmol), (E)-3-(4-hydroxy-3,5-dimethyl-phenyl)prop-2-enenitrile (378 mg, 2.18 mmol) and potassium carbonate (591 mg, 4.28 mmol) in DMF (10.0 mL) was stirred at rt for 2h. Water was added to the mixture and the precipitate was collected.

### Preparation of (E)-3-((4-(4-(2-cyanovinyl)-2,6-dimethylphenoxy)-5,6,7,8-tetrahydropyrido[1′,2′:1,5]pyrazolo[3,4-d]pyrimidin-2-yl)amino)bicyclo[1.1.1]pentane-1-carbonitrile (Compound 256)

The reaction mixture of Intermediate 256f (50 mg, 0.137 mmol), 3-aminobicyclo[1.1.1]pentane-1-carbonitrile (44.3 mg, 0.41 mmol), rac-BINAP-Pd-G3 (136 mg, 0.137 mmol) and cesium carbonate (223 mg, 0.68 mmol) in dioxane (3.0 mL) was degassed with Ar for 10 min. Then the reaction mixture was heated at 60° C. overnight. The reaction mixture was filtered through a syringe filter and purified using reverse phase HPLC, eluting with 5-100% acid modified MeCN/H₂O. The collected fractions were further purified using reverse phase HPLC, eluting with 5-100% neutral MeCN/H₂O. MS (m/z) 452.4 [M+H]+. ¹H NMR (400 MHz, Acetonitrile-d3) δ 7.50 (d, J=16.7 Hz, 1H), 7.41 (s, 2H), 6.09 (d, J=16.7 Hz, 1H), 4.25 (t, J=6.1 Hz, 2H), 3.22 (t, J=6.3 Hz, 2H), 2.15 (m, 16H).

### Example 257

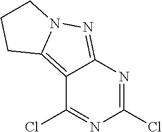 [SMILES: Clc1nc(Cl)c2c3n(nc2n1)CCC3]

2,4-dichloro-6,7-dihydro-5H-pyrrolo[1′,2′:1,5]pyrazolo[3,4-d]pyrimidine (Intermediate 257a) was prepared in a similar manner to 2,4-dichloro-5,6,7,8-tetrahydropyrido[1′,2′:1,5]pyrazolo[3,4-d]pyrimidine (Intermediate 256f) except that 4-chlorobutanoyl chloride was used instead of 5-chloropentanoyl chloride.

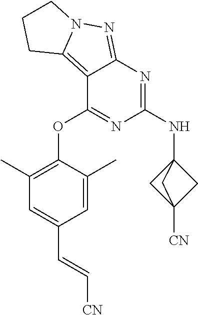 [SMILES: Cc1cc(/C=C/C#N)cc(C)c1Oc1nc(NC23CC(C#N)(C2)C3)nc2nn3c(c12)CCC3]

(E)-3-((4-(4-(2-cyanovinyl)-2,6-dimethylphenoxy)-6,7-dihydro-5H-pyrrolo[1′,2′:1,5]pyrazolo[3,4-d]pyrimidin-2-yl)amino)bicyclo[1.1.1]pentane-1-carbonitrile (Compound 257) was made in a similar manner to (E)-3-((4-(4-(2-cyanovinyl)-2,6-dimethylphenoxy)-5,6,7,8-tetrahydropyrido[1′,2′:1,5]pyrazolo[3,4-d]pyrimidin-2-yl)amino)bicyclo[1.1.1]pentane-1-carbonitrile (Compound 256) except that 2,4-dichloro-6,7-dihydro-5H-pyrrolo[1′,2′:1,5]pyrazolo[3,4-d]pyrimidine was used instead of 2,4-dichloro-5,6,7,8-tetrahydropyrido[1′,2′:1,5]pyrazolo[3,4-d]pyrimidine. MS (m/z) 438.4 [M+H]+. ¹H NMR (400 MHz, Acetonitrile-d3) δ 7.51 (d, J=16.6 Hz, 1H), 7.41 (s, 2H), 6.09 (d, J=16.7 Hz, 1H), 4.36-4.27 (m, 2H), 3.24 (t, J=7.4 Hz, 2H), 2.71 (p, J=7.4 Hz, 2H), 2.15 (s, 12H).

### Example 258

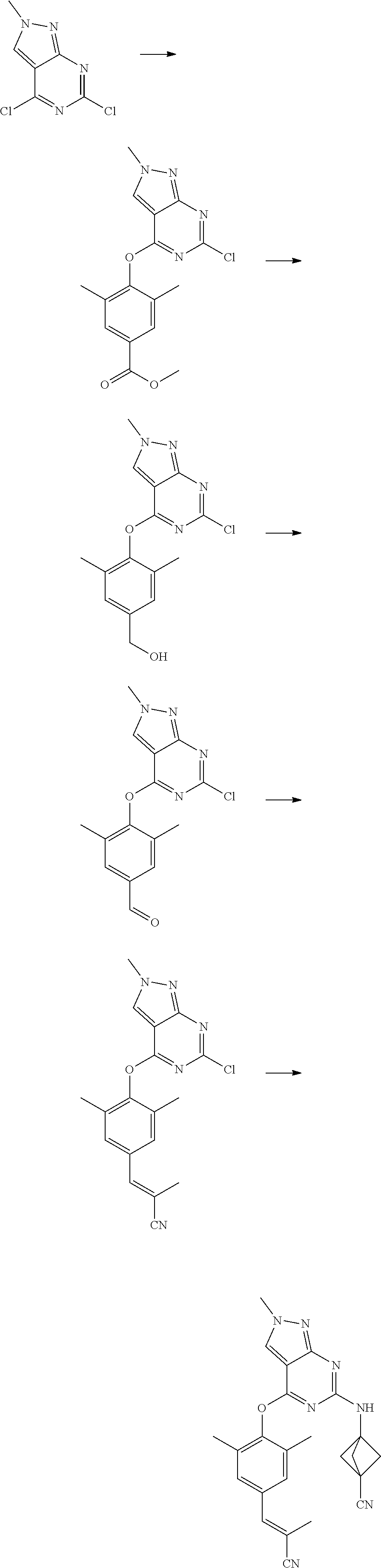

### Preparation of methyl 4-((6-chloro-2-methyl-2H-pyrazolo[3,4-d]pyrimidin-4-yl)oxy)-3,5-dimethylbenzoate (Intermediate 258a)

The reaction mixture of 4,6-dichloro-2-methyl-pyrazolo[3,4-d]pyrimidine (1 g, 4.93 mmol), methyl 4-hydroxy-3,5-dimethyl-benzoate (1.78 g, 9.85 mmol), potassium carbonate (2.06 g, 14.8 mmol) in DMF (10.0 mL) was stirred at rt for 5h. Water was added to the mixture and the precipitate was collected, dried under high vacuum and used in next step without purification.

### Preparation of (4-((6-chloro-2-methyl-2H-pyrazolo[3,4-d]pyrimidin-4-yl)oxy)-3,5-dimethylphenyl)methanol (Intermediate 258b)

To a suspension of methyl 4-((6-chloro-2-methyl-2H-pyrazolo[3,4-d]pyrimidin-4-yl)oxy)-3,5-dimethylbenzoate (350 mg, 1.01 mmol) in THF (3 mL) at 0° C., was added lithium aluminum hydride in THF (1.0 mol/L, 1.01 mL). The reaction mixture was stirred at 0° C. for 3h. To the reaction mixture was added water, extracted with EtOAc, dried over MgSO4, filtered, concentrated and purified with silica gel chromatography, eluting with 0-100% hexane/EtOAc.

### Preparation of 4-((6-chloro-2-methyl-2H-pyrazolo[3,4-d]pyrimidin-4-yl)oxy)-3,5-dimethylbenzaldehyde (Intermediate 258c)

The reaction mixture of (4-((6-chloro-2-methyl-2H-pyrazolo[3,4-d]pyrimidin-4-yl)oxy)-3,5-dimethylphenyl)methanol (260 mg, 0.816 mmol), Dess Martin periodinane (519 mg, 1.22 mmol) in DCM (3.0 mL) was stirred at rt for 3 h. The reaction mixture was quenched by adding 0.1M Na₂SO₃ followed by sat. NaHCO₃, and stirred at rt for 30 min. Then the mixture was extracted with EtOAc, the organic phase was separated, dried over MgSO4, filtered, concentrated and purified using silica gel chromatography, eluting with 0-80% hexane/EtOAc.

### Preparation of (E)-3-(4-((6-chloro-2-methyl-2H-pyrazolo[3,4-d]pyrimidin-4-yl)oxy)-3,5-dimethylphenyl)-2-methylacrylonitrile (Intermediate 258d)

To a solution of diethyl (1-cyanoethyl)phosphonate (154 mg, 0.81 mmol) in THF (3 mL) at 0° C., was added potassium tert-butoxide (90 mg, 0.81 mmol). The reaction mixture was stirred at 0° C. for 1 h. To the mixture was added the suspension of 4-(6-chloro-2-methyl-pyrazolo[3,4-d]pyrimidin-4-yl)oxy-3,5-dimethyl-benzaldehyde (170 mg, 0.537 mmol) in THE (1 mL). The reaction mixture was stirred at rt overnight. To the reaction mixture was added water, extracted with EtOAc, dried over MgSO4, filtered, concentrated, purified by silica gel chromatography, 0-100% hexane/EtOAc.

(E)-3-((4-(4-(2-cyanoprop-1-en-1-yl)-2,6-dimethylphenoxy)-2-methyl-2H-pyrazolo[3,4-d]pyrimidin-6-yl)amino)bicyclo[1.1.1]pentane-1-carbonitrile (Compound 258) was made in similar manner to Compound 256 except that (E)-3-(4-((6-chloro-2-methyl-2H-pyrazolo[3,4-d]pyrimidin-4-yl)oxy)-3,5-dimethylphenyl)-2-methylacrylonitrile was used instead of (E)-3-(4-((2-chloro-5,6,7,8-tetrahydropyrido[1′,2′:1,5]pyrazolo[3,4-d]pyrimidin-4-yl)oxy)-3,5-dimethylphenyl)acrylonitrile. MS (m/z) 426.3 [M+H]+. ¹H NMR (400 MHz, Acetonitrile-d3) δ 11.11 (s, 1H), 8.37 (s, 1H), 7.59 (s, 2H), 7.30 (s, 1H), 7.13 (d, J=1.8 Hz, 1H), 4.11 (s, 3H), 2.19 (s, 3H), 2.14 (m, 12H).

### Example 259

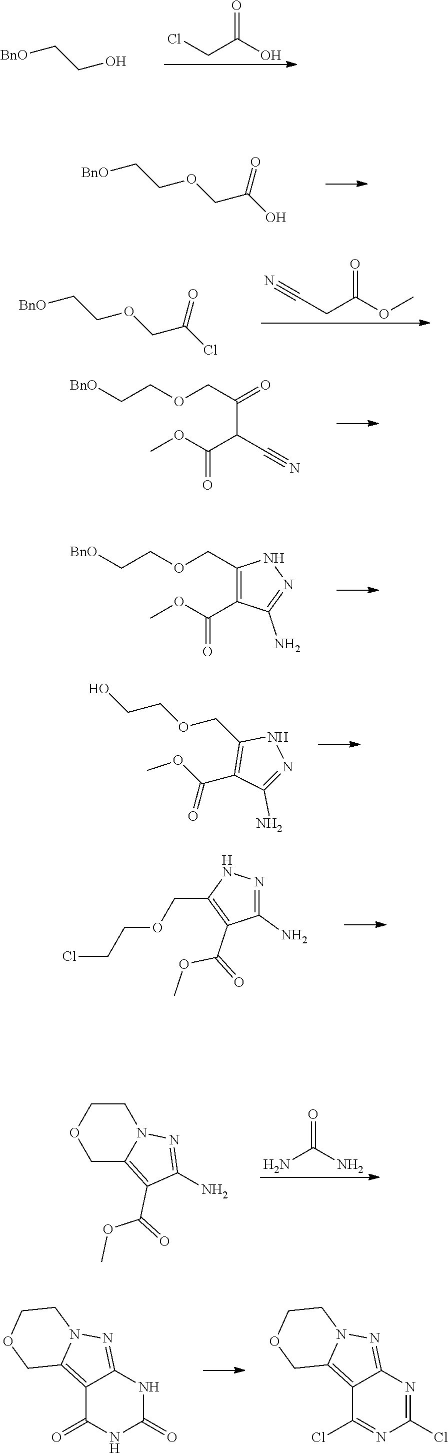

### Preparation of 2-(2-(benzyloxy)ethoxy)acetic acid (Intermediate 259a)

To a solution of NaH (24.3 g, 1.01 mol, 2.00 equiv, 60% in mineral oil) in anhydrous DMF (700 mL) was added benzyl glycol (77.0 g, 506 mmol, 1.00 equiv) dropwise at 0° C. under nitrogen atmosphere. The resulting mixture was stirred at room temperature for 1 h under nitrogen atmosphere. To the above mixture was added chloroacetic acid (50.2 g, 531 mmol, 1.05 equiv) in portions at 0° C. The resulting mixture was stirred at room temperature overnight. The reaction was quenched with sat. NaHCO₃ (aq.) at room temperature. The resulting mixture was extracted with EtOAc (3×1.00 L). The aqueous layer was acidified to pH 3-4 with conc. HCl. The resulting mixture was extracted with EtOAc (3×1.00 L). The combined organic layers were dried over anhydrous Na₂SO₄. After filtration, the filtrate was concentrated under reduced pressure to afford [2-(benzyloxy)ethoxy]acetic acid as a light yellow oil (98 g, 92.1% yield, 98.0% purity). The reaction was repeated seven times and a total of 726 g product was obtained. LCMS (ESI, m z): [M+1]=211.05.

### Preparation of 2-(2-(benzyloxy)ethoxy)acetyl chloride (Intermediate 259b)

To a solution of [2-(benzyloxy)ethoxy]acetic acid (77.0 g, 366 mmol, 1.00 equiv) and DMF (2.68 g, 36.6 mmol, 0.100 equiv) in DCM (1.00 L) was added oxalyl chloride (69.7 g, 549 mmol, 1.50 equiv) dropwise at 0° C. under nitrogen atmosphere. The resulting mixture was stirred at room temperature for 3 h under nitrogen atmosphere. The resulting mixture was concentrated under reduced pressure to afford [2-(benzyloxy)ethoxy]acetyl chloride as a yellow oil (90 g, crude), which was directly used in next step without further purification. The reaction was repeated ten times and a total of 810 g product was obtained. LCMS (ESI, m z): [M−Cl+OMe+1]=225.15.

### Preparation of methyl 4-(2-(benzyloxy)ethoxy)-2-cyano-3-oxobutanoate (Intermediate 259c)

To a solution of NaH (29.6 g, 739 mmol, 2.00 equiv, 60% in mineral oil) in anhydrous THE (500 mL) was added methyl cyanoacetate (36.6 g, 369 mmol, 1.00 equiv) dropwise at 0° C. under nitrogen atmosphere. The resulting mixture was stirred at room temperature for 1 h under nitrogen atmosphere. To the above mixture was added [2-(benzyloxy)ethoxy]acetyl chloride (84.5 g, 369 mmol, 1.00 equiv) in portions at 0° C. The resulting mixture was stirred at room temperature overnight. The reaction was quenched with sat. NaHCO₃ (aq.) at room temperature. The resulting mixture was extracted with PE (3×1.00 L). The aqueous layer was acidified to pH 3-4 with conc. HCl. The resulting mixture was extracted with EtOAc (3×2.00 L). The combined organic layers were dried over anhydrous Na₂SO₄. After filtration, the filtrate was concentrated under reduced pressure to afford methyl 4-[2-(benzyloxy)ethoxy]-2-cyano-3-oxobutanoate as a brown oil (118 g, crude), which was directly used in next step without further purification. The reaction was repeated nine times and a total of 942.5 g product was obtained. LCMS (ESI, m z): [M+1]=292.15.

### Preparation of methyl 3-amino-5-((2-(benzyloxy)ethoxy)methyl)-1H-pyrazole-4-carboxylate (Intermediate 259d)

To a stirred solution of methyl 4-[2-(benzyloxy)ethoxy]-2-cyano-3-oxobutanoate (75.0 g, 257 mmol, 1.00 equiv) in TFA (38.0 mL) was added Hydrazine monohydrate (25.8 g, 515 mmol, 2.00 equiv, 80%) dropwise at 0° C. under nitrogen atmosphere. The resulting mixture was stirred at 100° C. for 0.5 h under nitrogen atmosphere. The mixture was allowed to cool down to room temperature. The reaction was quenched with water at room temperature. The resulting mixture was extracted with EtOAc (3×500 mL). The combined organic layers were washed with brine (1×1.00 L), dried over anhydrous Na₂SO₄. After filtration, the filtrate was concentrated under reduced pressure. The residue was purified by silica gel column chromatography, eluted with PE/EA (1:2) to afford methyl 3-amino-5-{[2-(benzyloxy)ethoxy]methyl}-1H-pyrazole-4-carboxylate as a brown oil (37.0 g, 47.1% yield, 98.4% purity). The reaction was repeated five times and a total of 146 g product was obtained. LCMS (ESI, m z): [M+1]=306.05.

### Preparation of methyl 3-amino-5-((2-hydroxyethoxy)methyl)-1H-pyrazole-4-carboxylate (Intermediate 259e)

To a solution of methyl 3-amino-5-{[2-(benzyloxy)ethoxy]methyl}-1H-pyrazole-4-carboxylate (37.0 g, 121 mmol, 1.00 equiv) in 300 mL MeOH was added Pd/C (15.0 g, 10% Pd) under nitrogen atmosphere in a 500 mL round-bottom flask. The mixture was hydrogenated 45° C. for 4 h under hydrogen atmosphere using a hydrogen balloon, filtered through a Celite pad and concentrated under reduced pressure to afford methyl 3-amino-5-[(2-hydroxyethoxy)methyl]-1H-pyrazole-4-carboxylate as a light yellow solid (21.0 g, 80.5% yield, 84.2% purity). The reaction was repeated three times and a total of 53.8 g product was obtained. LCMS (ESI, m z): [M+1]=216.15.

### Preparation of methyl 3-amino-5-((2-chloroethoxy)methyl)-1H-pyrazole-4-carboxylate (Intermediate 259f)

A solution of methyl 3-amino-5-[(2-hydroxyethoxy)methyl]-1H-pyrazole-4-carboxylate (21.0 g, 97.6 mmol, 1.00 equiv) in thionyl chloride (100 mL) was stirred at 60° C. overnight under nitrogen atmosphere. The mixture was allowed to cool down to room temperature. The resulting mixture was concentrated under reduced pressure. The residue was quenched with water at room temperature. The mixture was basified to pH 7 with saturated NaHCO₃ (aq.). The resulting mixture was extracted with EtOAc (3×300 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (1:2) to afford methyl 3-amino-5-[(2-chloroethoxy) methyl]-1H-pyrazole-4-carboxylate as a light yellow solid (8.50 g, 37.3% yield, 89.9% purity). The reaction was repeated three times and a total of 40 g product was obtained. LCMS (ESI, m z): [M+1]=234.15.

### Preparation of 2,4-dichloro-7,8-dihydro-5H-pyrimido[4′,5′:3,4]pyrazolo[5,1-c][1,4]oxazine (Intermediate 259g)

2,4-dichloro-7,8-dihydro-5H-pyrimido[4′,5′:3,4]pyrazolo[5,1-c][1,4]oxazine was prepared in similar way to make 2,4-dichloro-5,6,7,8-tetrahydropyrido[1′,2′:1,5]pyrazolo[3,4-d]pyrimidine Intermediate 256e except that methyl 3-amino-5-((2-chloroethoxy)methyl)-1H-pyrazole-4-carboxylate was used instead of methyl 3-amino-5-(4-chlorobutyl)-1H-pyrazole-4-carboxylate. LCMS (ESI, m z): [M+1]=245.05.

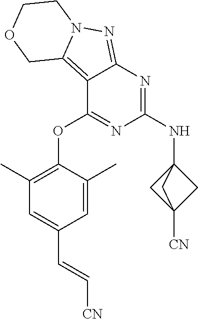 [SMILES: Cc1cc(/C=C/C#N)cc(C)c1Oc1nc(NC23CC(C#N)(C2)C3)nc2nn3c(c12)COCC3]

(E)-3-((4-(4-(2-cyanovinyl)-2,6-dimethylphenoxy)-7,8-dihydro-5H-pyrimido[4′,5′:3,4]pyrazolo[5,1-c][1,4]oxazin-2-yl)amino)bicyclo[1.1.1]pentane-1-carbonitrile (Compound 259) was made in similar manner to Compound 256, except that 2,4-dichloro-7,8-dihydro-5H-pyrimido[4′,5′:3,4]pyrazolo[5,1-c][1,4]oxazine was used instead of 2,4-dichloro-5,6,7,8-tetrahydropyrido[1′,2′:1,5]pyrazolo[3,4-d]pyrimidine. MS (m/z) 454.2 [M+H]+. ¹H NMR (400 MHz, Acetonitrile-d3) δ 7.52 (d, J=16.7 Hz, 1H), 7.45 (s, 2H), 6.12 (d, J=16.7 Hz, 1H), 5.23 (s, 2H), 4.34 (t, J=5.2 Hz, 2H), 4.24 (t, J=5.1 Hz, 2H), 2.27-2.05 (m, 12H).

### Example 260

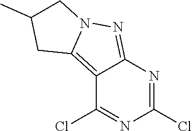

2,4-dichloro-6-methyl-6,7-dihydro-5H-pyrrolo[1′,2′:1,5]pyrazolo[3,4-d]pyrimidine (Intermediate 260a) was prepared in a similar manner to 2,4-dichloro-5,6,7,8-tetrahydropyrido[1′,2′:1,5]pyrazolo[3,4-d]pyrimidine.

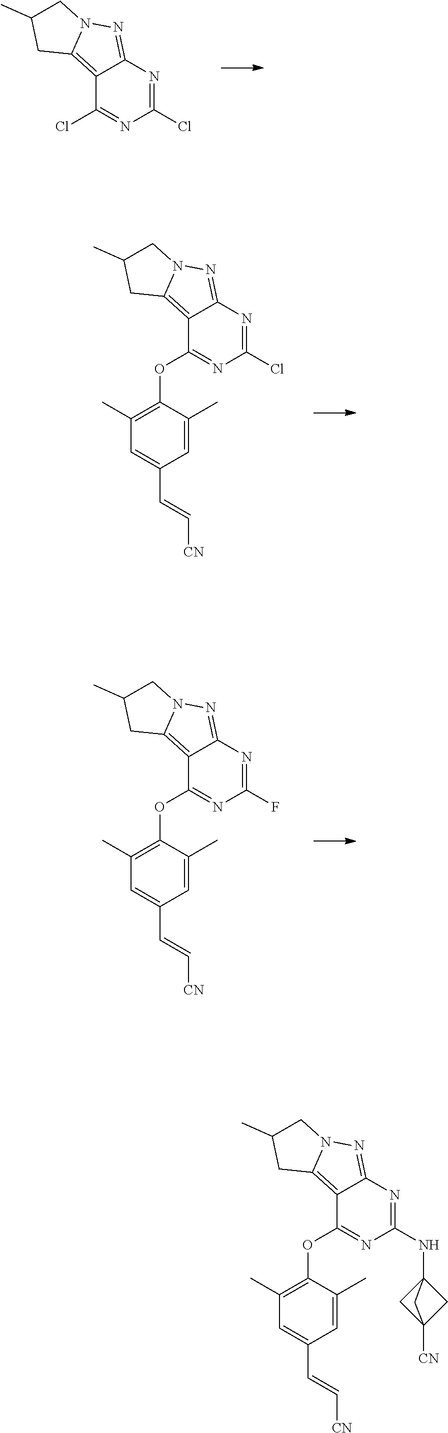 [SMILES: CC1Cc2c3c(Cl)nc(Cl)nc3nn2C1 ; Cc1cc(/C=C/C#N)cc(C)c1Oc1nc(Cl)nc2nn3c(c12)CC(C)C3 ; Cc1cc(/C=C/C#N)cc(C)c1Oc1nc(F)nc2nn3c(c12)CC(C)C3 ; Cc1cc(/C=C/C#N)cc(C)c1Oc1nc(NC23CC(C#N)(C2)C3)nc2nn3c(c12)CC(C)C3]

### Preparation of (E)-3-(4-((2-chloro-6-methyl-6,7-dihydro-5H-pyrrolo[1′,2′:1,5]pyrazolo[3,4-d]pyrimidin-4-yl)oxy)-3,5-dimethylphenyl)acrylonitrile (Intermediate 260b)

The reaction mixture of 2,4-dichloro-6-methyl-6,7-dihydro-5H-pyrrolo[1′,2′:1,5]pyrazolo[3,4-d]pyrimidine (100 mg, 0.411 mmol), (E)-3-(4-hydroxy-3,5-dimethyl-phenyl)prop-2-enenitrile (71 mg, 0.411 mmol) and potassium carbonate (172 mg, 1.23 mmol) in DMF (3.0 mL) was stirred at rt for 2h. Water was added to the mixture and the precipitate was collected.

### Preparation of (E)-3-(4-((2-fluoro-6-methyl-6,7-dihydro-5H-pyrrolo[1′,2′:1,5]pyrazolo[3,4-d]pyrimidin-4-yl)oxy)-3,5-dimethylphenyl)acrylonitrile (Intermediate 260c)

The reaction mixture of (E)-3-(4-((2-chloro-6-methyl-6,7-dihydro-5H-pyrrolo[1′,2′:1,5]pyrazolo[3,4-d]pyrimidin-4-yl)oxy)-3,5-dimethylphenyl)acrylonitrile (145 mg, 0.382 mmol), potassium fluoride (111 mg, 1.91 mmol), 1,4-diazabicyclo[2.2.2]octane (21.4 mg, 0.19 mmol) in NMP was heated at 70° C. overnight. Water was added to the mixture and the precipitate was collected.

### Preparation of (E)-3-((4-(4-(2-cyanovinyl)-2,6-dimethylphenoxy)-6-methyl-6,7-dihydro-5H-pyrrolo[1′,2′:1,5]pyrazolo[3,4-d]pyrimidin-2-yl)amino)bicyclo[1.1.1]pentane-1-carbonitrile (Compound 260)

The reaction mixture of (E)-3-(4-((2-fluoro-6-methyl-6,7-dihydro-5H-pyrrolo[1′,2′:1,5]pyrazolo[3,4-d]pyrimidin-4-yl)oxy)-3,5-dimethylphenyl)acrylonitrile (76 mg, 0.209 mmol), 3-aminobicyclo[1.1.1]pentane-1-carbonitrile (113 mg, 1.05 mmol), trifluoroacetic acid (0.16 mL, 2.09 mmol) in trifluoroethanol (1.0 mL) was stirred at 60° C. overnight. The reaction mixture was filtered through a syringe filter and purified by reverse phase HPLC, eluting with acid modified MeCN/H₂O. MS (m/z) 452.2 [M+H]+. ¹H NMR (400 MHz, Acetonitrile-d3) δ 7.53 (d, J=16.7 Hz, 1H), 7.46 (s, 2H), 6.13 (d, J=16.7 Hz, 1H), 4.51 (ddd, J=11.9, 8.2, 1.3 Hz, 1H), 3.96 (ddt, J=12.0, 6.5, 1.4 Hz, 1H), 3.50 (ddd, J=17.0, 8.4, 1.3 Hz, 1H), 3.30 (ddt, J=14.7, 8.1, 6.4 Hz, 1H), 2.92 (ddt, J=17.1, 6.1, 1.4 Hz, 1H), 2.18 (s, 6H), 2.11 (s, 6H), 1.34 (d, J=6.9 Hz, 3H).

### Example 261

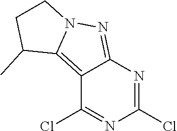

2,4-dichloro-5-methyl-6,7-dihydro-5H-pyrrolo[1′,2′:1,5]pyrazolo[3,4-d]pyrimidine (Intermediate 261a) was prepared in a similar manner to 2,4-dichloro-5,6,7,8-tetrahydropyrido[1′,2′:1,5]pyrazolo[3,4-d]pyrimidine.

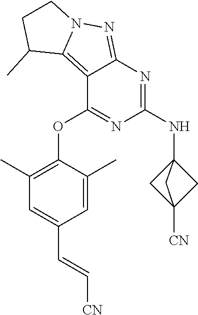 [SMILES: Cc1cc(/C=C/C#N)cc(C)c1Oc1nc(NC23CC(C#N)(C2)C3)nc2nn3c(c12)C(C)CC3]

(E)-3-((4-(4-(2-cyanovinyl)-2,6-dimethylphenoxy)-5-methyl-6,7-dihydro-5H-pyrrolo[1′,2′:1,5]pyrazolo[3,4-d]pyrimidin-2-yl)amino)bicyclo[1.1.1]pentane-1-carbonitrile (Compound 261) was made in similar manner to Compound 260, except that 2,4-dichloro-5-methyl-6,7-dihydro-5H-pyrrolo[1′,2′:1,5]pyrazolo[3,4-d]pyrimidine was used instead of 2,4-dichloro-6-methyl-6,7-dihydro-5H-pyrrolo[1′,2′:1,5]pyrazolo[3,4-d]pyrimidine. MS (m/z) 452.3 [M+H]+. ¹H NMR (400 MHz, Acetonitrile-d3) δ 7.53 (d, J=16.7 Hz, 1H), 7.47 (s, 2H), 6.13 (d, J=16.7 Hz, 1H), 4.43 (dddd, J=12.0, 9.1, 4.3, 1.4 Hz, 1H), 4.33 (dt, J=12.0, 7.9 Hz, 1H), 3.79-3.67 (m, 1H), 2.98 (dtd, J=12.9, 8.5, 4.4 Hz, 1H), 2.33-2.40 (m, 1H), 2.18 (d, J=2.4 Hz, 6H), 2.11 (s, 6H), 1.56 (d, J=7.1 Hz, 3H).

### Example 262

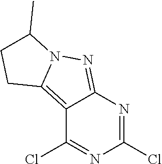

2,4-dichloro-7-methyl-6,7-dihydro-5H-pyrrolo[1′,2′:1,5]pyrazolo[3,4-d]pyrimidine (Intermediate 262a) was prepared in a similar manner to 2,4-dichloro-5,6,7,8-tetrahydropyrido[1′,2′:1,5]pyrazolo[3,4-d]pyrimidine.

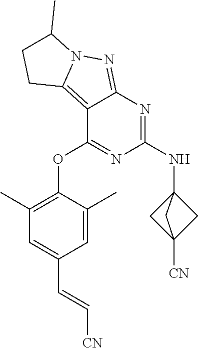 [SMILES: Cc1cc(/C=C/C#N)cc(C)c1Oc1nc(NC23CC(C#N)(C2)C3)nc2nn3c(c12)CCC3C]

(E)-3-((4-(4-(2-cyanovinyl)-2,6-dimethylphenoxy)-7-methyl-6,7-dihydro-5H-pyrrolo[1′,2′:1,5]pyrazolo[3,4-d]pyrimidin-2-yl)amino)bicyclo[1.1.1]pentane-1-carbonitrile (Compound 262) was made in similar manner to Compound 260, except that 2,4-dichloro-7-methyl-6,7-dihydro-5H-pyrrolo[1′,2′:1,5]pyrazolo[3,4-d]pyrimidine was used instead of 2,4-dichloro-6-methyl-6,7-dihydro-5H-pyrrolo[1′,2′:1,5]pyrazolo[3,4-d]pyrimidine. MS (m/z) 452.3 [M+H]+. ¹H NMR (400 MHz, Acetonitrile-d3) δ 11.17 (bs, 1H), 7.53 (d, J=16.7 Hz, 1H), 7.47 (s, 2H), 6.13 (d, J=16.7 Hz, 1H), 4.75-4.61 (m, 1H), 3.38-3.16 (m, 2H), 3.03-2.89 (m, 1H), 2.36 (ddt, J=13.3, 9.4, 6.8 Hz, 1H), 2.19 (d, J=3.3 Hz, 6H), 2.11 (s, 6H), 1.59 (d, J=6.4 Hz, 3H).

### Example 263

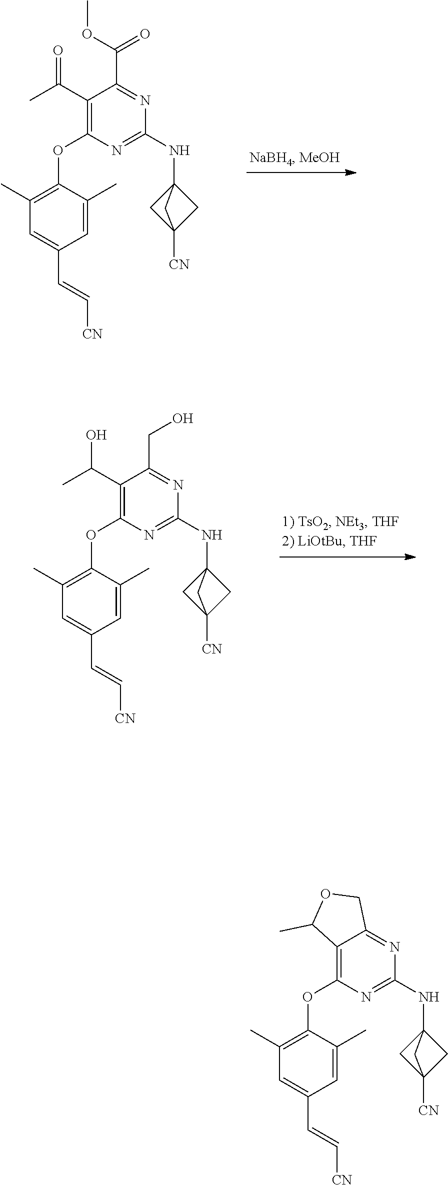 [SMILES: COC(=O)c1nc(NC23CC(C#N)(C2)C3)nc(Oc2c(C)cc(/C=C/C#N)cc2C)c1C(C)=O ; Cc1cc(/C=C/C#N)cc(C)c1Oc1nc(NC23CC(C#N)(C2)C3)nc(CO)c1C(C)O ; Cc1cc(/C=C/C#N)cc(C)c1Oc1nc(NC23CC(C#N)(C2)C3)nc2c1C(C)OC2]

### Preparation of (E)-3-((4-(4-(2-cyanovinyl)-2,6-dimethylphenoxy)-5-(1-hydroxyethyl)-6-(hydroxymethyl)pyrimidin-2-yl)amino)bicyclo[1.1.1]pentane-1-carbonitrile (Intermediate 263a)

To a solution of methyl (E)-5-acetyl-2-((3-cyanobicyclo[1.1.1]pentan-1-yl)amino)-6-(4-(2-cyanovinyl)-2,6-dimethylphenoxy)pyrimidine-4-carboxylate (Intermediate 136d) (173 mg, 0.38 mmol, 1 equiv) in MeOH (9 mL) at 0° C. was added NaBH₄ (21 mg, 0.57 mmol, 1.5 equiv). The reaction was warmed to room temperature and stirred for 1 hour, before being quenched with water. The resulting crude mixture was purified by RP-HPLC (10-90% ACN/water) and dried overnight on the lyo. MS (m/z): 432.00 [M+H]⁺.

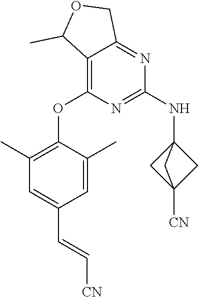 [SMILES: Cc1cc(/C=C/C#N)cc(C)c1Oc1nc(NC23CC(C#N)(C2)C3)nc2c1C(C)OC2]

### Preparation of (E)-3-((4-(4-(2-cyanovinyl)-2,6-dimethylphenoxy)-5-methyl-5,7-dihydrofuro[3,4-d]pyrimidin-2-yl)amino)bicyclo[1.1.1]pentane-1-carbonitrile (Compound 263)

To a solution of (E)-3-((4-(4-(2-cyanovinyl)-2,6-dimethylphenoxy)-5-(1-hydroxyethyl)-6-(hydroxymethyl)pyrimidin-2-yl)amino)bicyclo[1.1.1]pentane-1-carbonitrile (Intermediate 263a) (9.2 mg, 0.02 mmol, 1 equiv) and NEt₃ (15 uL, 0.11 mmol, 5 equiv) in THE (0.5 mL) was added pTsO₂ (14 mg, 0.04 mmol, 2 equiv) at 0° C. The reaction was warmed to room temperature and stirred for 2 hours. The reaction was diluted with an additional 1 mL of THE and LiOtBu (1M in THF, 64 μL, 0.064 mmol, 3 equiv)) was added dropwise. The reaction mixture was stirred for 2 h at room temperature before being diluted with ACN/water, filtered, and purified by RP-HPLC (10-90% ACN/water with TFA). MS (m/z): 414.02 [M+H]⁺. 1H NMR (400 MHz, Acetonitrile-d3) δ 7.99 (s, 1H), 7.48 (d, J=16.7 Hz, 1H), 7.40 (s, 2H), 6.07 (d, J=16.7 Hz, 1H), 5.44 (qdd, J=6.3, 2.9, 1.8 Hz, 1H), 4.91 (dd, J=14.8, 3.0 Hz, 1H), 4.81 (dd, J=14.8, 1.8 Hz, 1H), 2.11 (s, 6H), 2.06 (s, 6H), 1.56 (d, J=6.3 Hz, 3H).

### Example 264

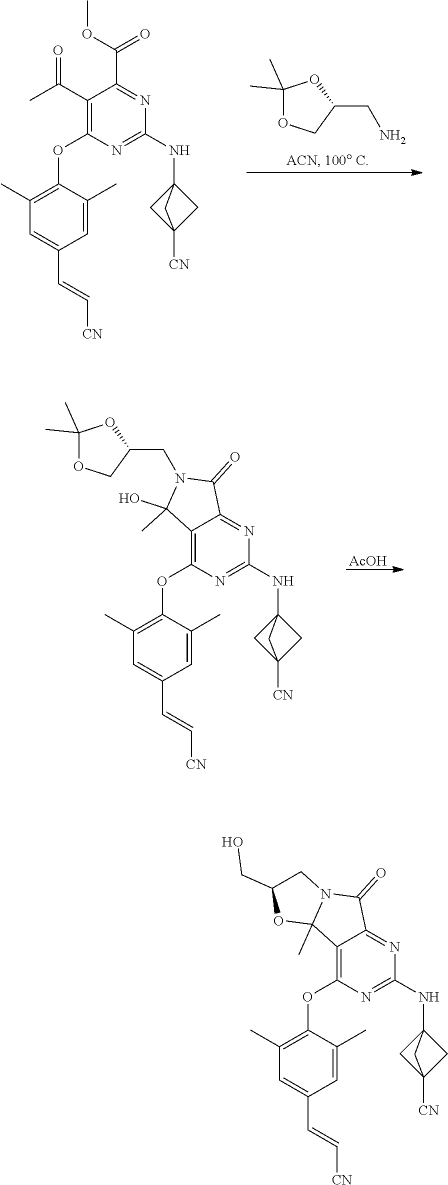

### Preparation of 3-(((6R)-4-(4-((E)-2-cyanovinyl)-2,6-dimethylphenoxy)-6-(hydroxymethyl)-4b-methyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 264)

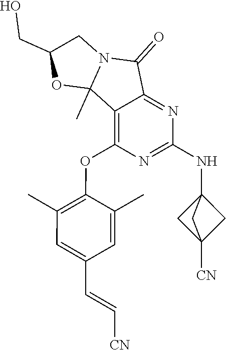

To a solution of methyl (E)-5-acetyl-2-((3-cyanobicyclo[1.1.1]pentan-1-yl)amino)-6-(4-(2-cyanovinyl)-2,6-dimethylphenoxy)pyrimidine-4-carboxylate (Intermediate 236d) (35 mg, 0.077 mmol, 1 equiv) in ACN (0.75 mL) was added (R)-(2,2-dimethyl-1,3-dioxolan-4-yl)methanamine (100 mg, 0.77 mg, 10 equiv). The reaction mixture was stirred at 100° C. for 14 h and used directly in the next step. AcOH (0.14 mL) and 2M aqueous HCl (0.77 mL) were added and the reaction mixture stirred at room temperature for 2 hours. The crude product was purified by RP-HIPLC (10-90% ACN/water with TFA). MS (m/z): 498.90 [M+H]⁺. 1H NMR (400 MHz, CD3CN) δ 7.48 (d, J=16.7 Hz, 1H), 7.41 (d, J=2.5 Hz, 2H), 6.85 (s, 1H), 6.07 (d, J=16.7 Hz, 1H), 4.26-4.22 (m, 1H), 4.22-4.18 (m, 1H), 3.76 (ddt, J=6.1, 4.1, 2.2 Hz, 2H), 3.51 (dd, J=14.0, 2.3 Hz, 1H), 2.13 (d, J=2.7 Hz, 3H), 2.11 (s, 3H), 2.04 (s, 6H), 1.90 (s, 3H).

### Example 265

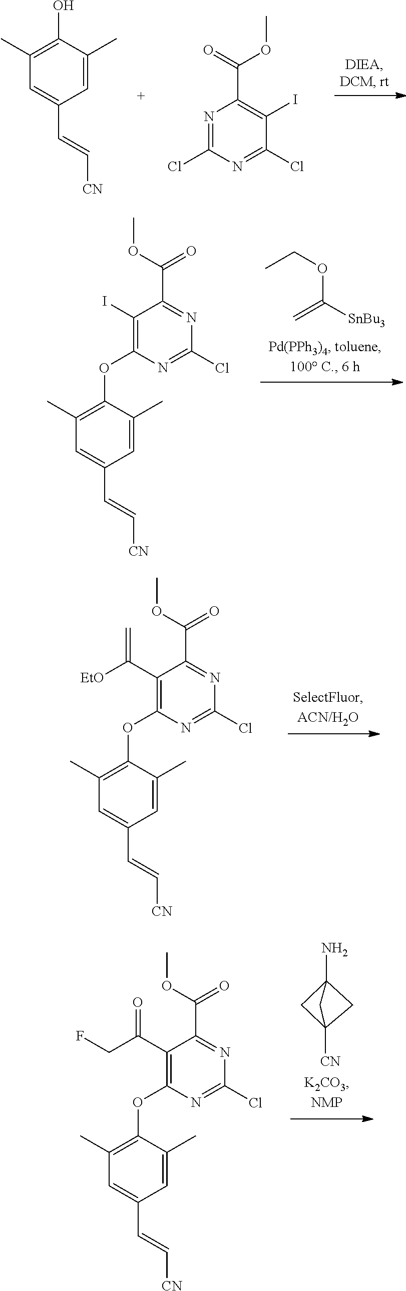

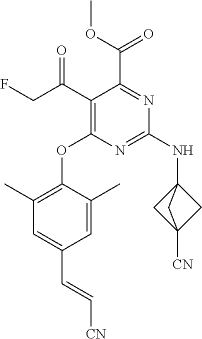 [SMILES: COC(=O)c1nc(NC23CC(C#N)(C2)C3)nc(Oc2c(C)cc(/C=C/C#N)cc2C)c1C(=O)CF]

### Step 1, preparation of methyl (E)-2-chloro-6-(4-(2-cyanovinyl)-2,6-dimethylphenoxy)-5-iodopyrimidine-4-carboxylate (Intermediate 265a)

To a mixture of (E)-3-(4-hydroxy-3,5-dimethylphenyl)acrylonitrile (85.0 g, 490.7 mmol, 1.0 eq) in DCM (850 mL) was added DIEA (126.8 g, 981.4 mmol, 2.0 eq) and methyl 2,6-dichloro-5-iodopyrimidine-4-carboxylate (179.7 g, 539.8 mmol, 1.1 eq) at room temperature, the mixture was stirred at room temperature overnight. After completion, H2O (500 mL) was added and the reaction mixture was extracted with DCM (500 mL×3), washed with H2O (500 mL×3) and brine (500 mL×3), dried over Na₂SO₄, filtered and concentrated. The crude was triturated with MTBE (300 mL), filtered and dried to give compound 7 (100.0 g, 43.4%) as a white solid. MS (m/z): 470.0 [M+H]+. 1H NMR (300 MHz, CDCl3): δ ppm 7.35 (d, J=16.5 Hz, 1H), 7.22 (s, 2H), 5.86 (d, J=16.8 Hz, 1H), 4.05 (s, 3H), 2.13 (s, 6H).

### Step 2, preparation of methyl (E)-2-chloro-6-(4-(2-cyanovinyl)-2,6-dimethylphenoxy)-5-(1-ethoxyvinyl)pyrimidine-4-carboxylate (Intermediate 265b)

To a mixture of compound 7 (120.0 g, 255.5 mmol, 1.0 eq) and Pd(PPh3)4 (1.5 g, 1.3 mmol, 0.05 eq) in toluene (720 mL) was added compound 8 (101.5 g, 281.1 mmol, 1.1 eq) at room temperature. The mixture was stirred at 110° C. for 6 h under N2. After completion, the mixture was cooled to room temperature, filtered and concentrated. The crude was purified by silica gel column chromatography (Petroleum ether: EtOAc=1: 1) to give G-13167-1 (Bellen00071356-N) (50.0 g, 47.3%) as a white solid. MS (m/z): 414.1 ([M+H]+). 1H NMR (300 MHz, DMSO-d6): δ ppm 7.61 (d, J=16.8 Hz, 1H), 7.50 (s, 2H), 6.44 (d, J=16.8 Hz, 1H), 4.72 (dd, J=16.5 Hz, 3.0 Hz, 2H), 3.94-3.83 (m, 5H), 2.07 (s, 6H), 1.26 (t, J=7.2 Hz, 3H).

### Step 3, preparation of methyl (E)-2-chloro-6-(4-(2-cyanovinyl)-2,6-dimethylphenoxy)-5-(2-fluoroacetyl)pyrimidine-4-carboxylate (Intermediate 265c)

To a solution of methyl (E)-2-chloro-6-(4-(2-cyanovinyl)-2,6-dimethylphenoxy)-5-(1-ethoxyvinyl)pyrimidine-4-carboxylate (Intermediate 265b) (300 mg, 0.724 mmol, 1 equiv) in ACN/water (6 mL, 2:1) was added SelectFluor (0.642 g, 1.81 mmol, 2.5 equv). The reaction was stirred at room temperature for 2 hours. The reaction mixture was subsequently diluted with EtOAc/water, and extracted twice with EtOAc. The combined organic layers were dried over MgSO₄, filter, conc. in vacuo and purified by silica gel chromatography (0-75% EtOAc/hexanes). MS (m/z): 403.89 [M+H]⁺.

### Step 4, preparation of methyl (E)-2-((3-cyanobicyclo[1.1.1]pentan-1-yl)amino)-6-(4-(2-cyanovinyl)-2,6-dimethylphenoxy)-5-(2-fluoroacetyl)pyrimidine-4-carboxylate (Intermediate 265d)

A solution of methyl (E)-2-((3-cyanobicyclo[1.1.1]pentan-1-yl)amino)-6-(4-(2-cyanovinyl)-2,6-dimethylphenoxy)-5-(2-fluoroacetyl)pyrimidine-4-carboxylate (105 mg, 0.26 mmol, 1 equiv) and 3-aminobicyclo[1.1.1]pentane-1-carbonitrile (56 mg, 0.52 mmol, 2 equiv) in NMP (1.2 mL) was stirred at room temperature overnight. The reaction mixture was subsequently diluted with EtOAc/water, and extracted twice with EtOAc. The combined organic layers were dried over MgSO₄, filter, conc. in vacuo and purified by silica gel chromatography (0-70% EtOAc/hexanes). MS (m/z): 475.90 [M+H]⁺.

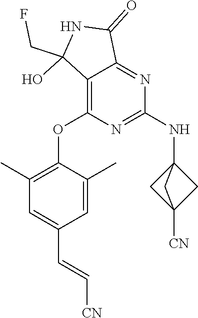 [SMILES: Cc1cc(/C=C/C#N)cc(C)c1Oc1nc(NC23CC(C#N)(C2)C3)nc2c1C(O)(CF)NC2=O]

### Preparation of (E)-3-((4-(4-(2-cyanovinyl)-2,6-dimethylphenoxy)-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile (Compound 265)

MeOH (0.5 mL) was added to methyl (E)-2-((3-cyanobicyclo[1.1.1]pentan-1-yl)amino)-6-(4-(2-cyanovinyl)-2,6-dimethylphenoxy)-5-(2-fluoroacetyl)pyrimidine-4-carboxylate (Intermediate 265d) (25 mg, 0.053 mmol, 1 equiv), ammonium acetate (41 mg, 0.53 mmol, 10 equiv) and sodium cyanoborohydride (33 mg, 0.53 mmol, 10 equiv) and the resulting reaction mixture was stirred at 100° C. for 1 hour. The crude product was purified by RP-HPLC (10-90% ACN/water). MS (m/z): 460.90 [M+H]⁺.

1H NMR (400 MHz, DMSO-d6) δ 9.53 (s, 1H), 8.72 (s, 1H), 7.67-7.43 (m, 3H), 6.97 (s, 1H), 6.44 (d, J=16.6 Hz, 1H), 4.71 (ddd, J=87.1, 47.1, 9.1 Hz, 2H), 2.10 (s, 3H), 2.05 (s, 3H), 1.97 (s, 6H).

### Example 266

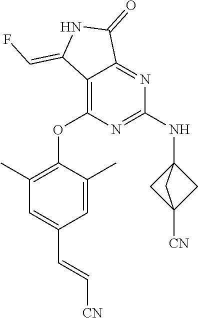 [SMILES: Cc1cc(/C=C/C#N)cc(C)c1Oc1nc(NC23CC(C#N)(C2)C3)nc2c1/C(=C/F)NC2=O]

### Preparation of 3-(((Z)-4-(4-((E)-2-cyanovinyl)-2,6-dimethylphenoxy)-5-(fluoromethylene)-7-oxo-6,7-dihydro-5H-pyrrolo[3,4-d]pyrimidin-2-yl)amino)bicyclo[1.1.1]pentane-1-carbonitrile (Compound 266)

A solution of (E)-3-((4-(4-(2-cyanovinyl)-2,6-dimethylphenoxy)-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile (5 mg, 0.011 mmol, 1 equiv) in acetic acid (0.5 mL) was stirred at 110° C. for 3 h. The reaction mixture was diluted with ACN/water and purified by RP-HPLC (10-90% ACN/water with TFA). MS (m/z): 442.90 [M+H]⁺. 1H NMR (400 MHz, DMSO-d6) δ 11.46 (s, 1H), 8.83 (m, 1H), 7.71-7.53 (m, 3H), 7.42 (d, J=76.1 Hz, 1H), 6.45 (d, J=16.7 Hz, 1H), 2.09 (s, 6H), 1.98 (s, 6H).

19F NMR (376 MHz, DMSO-d6) δ −74.90 (s, 3F), −145.13 (d, J=76.3 Hz, 1F).

### Example 267

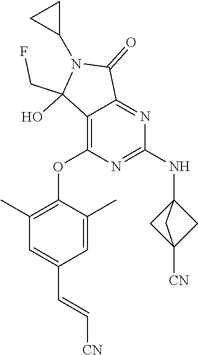 [SMILES: Cc1cc(/C=C/C#N)cc(C)c1Oc1nc(NC23CC(C#N)(C2)C3)nc2c1C(O)(CF)N(C1CC1)C2=O]

### Preparation of (E)-3-((4-(4-(2-cyanovinyl)-2,6-dimethylphenoxy)-6-cyclopropyl-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile (Compound 267)

Compound 267 was prepared using the procedure described for the synthesis of Compound 265 with the modification that cyclopropylamine was used in place of ammonium acetate. MS (m/z): 500.91 [M+H]⁺. ¹H NMR (400 MHz, DMSO-d6) δ 8.78 (s, 1H), 7.70-7.41 (m, 3H), 7.19-7.06 (m, 1H), 6.44 (d, J=16.7 Hz, 1H), 5.06-4.80 (m, 2H), 2.10 (s, 3H), 2.06 (s, 3H), 1.96 (s, 6H), 1.33 (ddt, J=10.7, 6.6, 4.4 Hz, 1H), 0.94-0.67 (m, 4H).

### Example 268

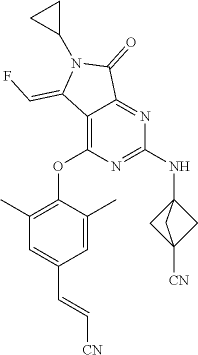 [SMILES: Cc1cc(/C=C/C#N)cc(C)c1Oc1nc(NC23CC(C#N)(C2)C3)nc2c1/C(=C/F)N(C1CC1)C2=O]

### Preparation of 3-(((Z)-4-(4-((E)-2-cyanovinyl)-2,6-dimethylphenoxy)-6-cyclopropyl-5-(fluoromethylene)-7-oxo-6,7-dihydro-5H-pyrrolo[3,4-d]pyrimidin-2-yl)amino)bicyclo[1.1.1]pentane-1-carbonitrile (Compound 268)

Compound 268 was prepared using the procedure described for the synthesis of Compound 266 with the modification that (E)-3-((4-(4-(2-cyanovinyl)-2,6-dimethylphenoxy)-6-cyclopropyl-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile was used in place of (E)-3-((4-(4-(2-cyanovinyl)-2,6-dimethylphenoxy)-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile. MS (m/z): 482.90 [M+H]⁺. 1H NMR (400 MHz, Acetonitrile-d3) δ 7.45 (d, J=77.1 Hz, 1H), 7.49 (d, J=16.7 Hz, 1H), 7.41 (s, 2H), 6.76 (s, 1H), 6.07 (d, J=16.7 Hz, 1H), 2.97 (tdd, J=6.4, 4.2, 3.3 Hz, 1H), 2.11 (s, 6H), 2.06 (s, 6H), 1.08-0.93 (m, 4H). 19F NMR (376 MHz, Acetonitrile-d3) δ −76.92 (s, 3F), −145.89 (d, J=77.3 Hz, 1F)

### Example 269

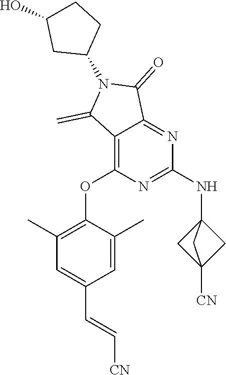 [SMILES: C=C1c2c(Oc3c(C)cc(/C=C/C#N)cc3C)nc(NC34CC(C#N)(C3)C4)nc2C(=O)N1[C@H]1CC[C@@H](O)C1]

### Preparation of 3-((4-(4-((E)-2-cyanovinyl)-2,6-dimethylphenoxy)-6-((1S,3R)-3-hydroxycyclopentyl)-5-methylene-7-oxo-6,7-dihydro-5H-pyrrolo[3,4-d]pyrimidin-2-yl)amino)bicyclo[1.1.1]pentane-1-carbonitrile (Compound 269)

3-((4-(4-((E)-2-cyanovinyl)-2,6-dimethylphenoxy)-5-hydroxy-6-((1S,3R)-3-hydroxycyclopentyl)-5-methyl-7-oxo-6,7-dihydro-5H-pyrrolo[3,4-d]pyrimidin-2-yl)amino)bicyclo[1.1.1]pentane-1-carbonitrile (Intermediate 269a) was prepared using the procedure described for the synthesis of Compound 235 with the modification that 3-aminocyclopentanol was used in place of (R)-3-amino-2-methoxypropan-1-ol. The reaction was heated for an additional hour at 150° C. MS (m/z): 526.90 [M+H]⁺.

Compound 269 was prepared using the procedure described for the synthesis of Compound 266 with the modification that 3-((4-(4-((E)-2-cyanovinyl)-2,6-dimethylphenoxy)-5-hydroxy-6-((1S,3R)-3-hydroxycyclopentyl)-5-methyl-7-oxo-6,7-dihydro-5H-pyrrolo[3,4-d]pyrimidin-2-yl)amino)bicyclo[1.1.1]pentane-1-carbonitrile was used in place of (E)-3-((4-(4-(2-cyanovinyl)-2,6-dimethylphenoxy)-5-(fluoromethyl)-5-hydroxy-7-oxo-6,7-dihydro-5H-pyrrolo[3,4-d]pyrimidin-2-yl)amino)bicyclo[1.1.1]pentane-1-carbonitrile. MS (m/z): 508.90 [M+H]⁺. 1H NMR (400 MHz, DMSO-d6) δ 8.84 (s, 1H), 7.70-7.53 (m, 3H), 6.45 (d, J=16.6 Hz, 1H), 5.67 (d, J=2.0 Hz, 1H), 5.45 (d, J=1.9 Hz, 1H), 4.77 (p, J=9.0 Hz, 1H), 4.19 (t, J=5.1 Hz, 1H), 2.36-2.24 (m, 1H), 2.18 (ddd, J=13.5, 10.0, 6.2 Hz, 1H), 2.09 (s, 6H), 2.03 (td, J=8.5, 4.2 Hz, 2H), 1.98 (s, 6H), 1.87-1.67 (m, 4H).

### Example 270

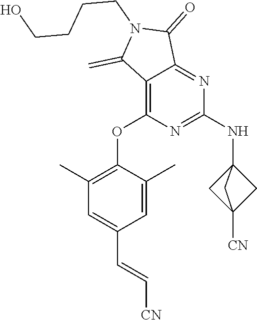 [SMILES: C=C1c2c(Oc3c(C)cc(/C=C/C#N)cc3C)nc(NC34CC(C#N)(C3)C4)nc2C(=O)N1CCCCO]

### Preparation of (E)-3-((4-(4-(2-cyanovinyl)-2,6-dimethylphenoxy)-6-(4-hydroxybutyl)-5-methylene-7-oxo-6,7-dihydro-5H-pyrrolo[3,4-d]pyrimidin-2-yl)amino)bicyclo[1.1.1]pentane-1-carbonitrile (Compound 270)

Compound 270 was prepared using the procedure described for the synthesis of Compound 235 with the modification that 4-aminobutan-1-ol was used in place of (R)-3-amino-2-methoxypropan-1-ol. The reaction was heated for an additional hour at 170° C. MS (m/z): 497.10 [M+H]⁺. 1H NMR (400 MHz, DMSO-d6) δ 8.87 (s, 1H), 7.69-7.53 (m, 3H), 6.45 (d, J=16.7 Hz, 1H), 5.38 (d, J=1.9 Hz, 1H), 5.23 (d, J=2.0 Hz, 1H), 4.43 (t, J=5.1 Hz, 1H), 3.77 (t, J=7.2 Hz, 2H), 3.46-3.36 (m, 3H), 2.09 (s, 6H), 1.98 (s, 6H), 1.64 (p, J=7.2 Hz, 2H), 1.45 (p, J=6.4 Hz, 2H).

### Example 271

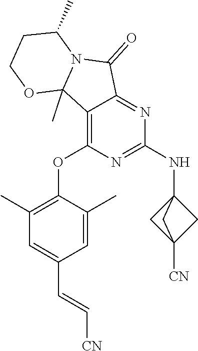 [SMILES: Cc1cc(/C=C/C#N)cc(C)c1Oc1nc(NC23CC(C#N)(C2)C3)nc2c1C1(C)OCC[C@H](C)N1C2=O]

### Preparation of 3-(((8S)-4-(4-((E)-2-cyanovinyl)-2,6-dimethylphenoxy)-4b,8-dimethyl-10-oxo-4b,7,8,10-tetrahydro-6H-pyrimido[5′,4′:3,4]pyrrolo[2,1-b][1,3]oxazin-2-yl)amino)bicyclo[11.1.1]pentane-1-carbonitrile (Compound 271)

Compound 271 was prepared using the procedure described for the synthesis of Compound 235 with the modification that (S)-3-aminobutan-1-ol was used in place of (R)-3-amino-2-methoxypropan-1-ol. MS (m/z): 497.20 [M+H]⁺. 1H NMR (400 MHz, DMSO-d6) δ 8.74 (s, 1H), 7.63 (d, J=16.6 Hz, 1H), 7.57 (s, 2H), 6.44 (d, J=16.6 Hz, 1H), 4.68-4.52 (m, 1H), 4.30 (t, J=11.8 Hz, 1H), 3.92-3.74 (m, 1H), 2.10 (d, J=2.4 Hz, 3H), 2.04 (s, 3H), 1.98 (s, 3H), 1.96 (s, 5H), 1.89 (s, 1H), 1.72 (d, J=6.7 Hz, 1H), 1.61-1.49 (m, 1H), 1.45 (d, J=7.2 Hz, 2H).

### Example 272

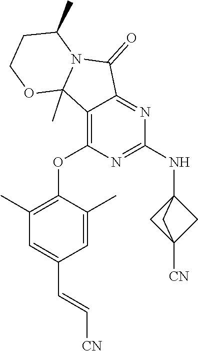 [SMILES: Cc1cc(/C=C/C#N)cc(C)c1Oc1nc(NC23CC(C#N)(C2)C3)nc2c1C1(C)OCC[C@@H](C)N1C2=O]

### Preparation of 3-(((8R)-4-(4-((E)-2-cyanovinyl)-2,6-dimethylphenoxy)-4b,8-dimethyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 272)

Compound 272 was prepared using the procedure described for the synthesis of Compound 235 with the modification that (R)-3-aminobutan-1-ol was used in place of (R)-3-amino-2-methoxypropan-1-ol. MS (m/z): 497.00 [M+H]⁺. 1H NMR (400 MHz, DMSO-d6) δ 8.76 (s, 1H), 7.64 (d, J=16.6 Hz, 1H), 7.58 (s, 2H), 6.45 (d, J=16.7 Hz, 1H), 4.67-4.58 (m, 1H), 4.31 (t, J=11.7 Hz, 1H), 3.84-3.75 (m, 1H), 2.08-1.81 (m, 15H), 1.73 (m, 1H), 1.57 (m, 1H), 1.46 (d, J=7.3 Hz, 3H).

### Example 273

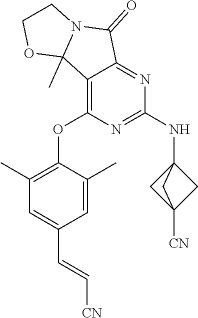 [SMILES: Cc1cc(/C=C/C#N)cc(C)c1Oc1nc(NC23CC(C#N)(C2)C3)nc2c1C1(C)OCCN1C2=O]

### Preparation of (E)-3-((4-(4-(2-cyanovinyl)-2,6-dimethylphenoxy)-4b-methyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 273)

Compound 273 was prepared using the procedure described for the synthesis of Compound 235 with the modification that 2-aminoethan-1-ol was used in place of (R)-3-amino-2-methoxypropan-1-ol. MS (m/z): 468.95 [M+H]⁺. 1H NMR (400 MHz, DMSO-d6) δ 8.82 (s, 1H), 7.70-7.47 (m, 3H), 6.45 (d, J=16.7 Hz, 1H), 4.29 (dd, J=8.2, 5.4 Hz, 1H), 4.18 (q, J=7.5 Hz, 1H), 3.89 (ddd, J=12.5, 7.7, 5.2 Hz, 1H), 3.49 (ddd, J=11.1, 7.6, 6.3 Hz, 1H), 3.35 (d, J=6.4 Hz, 1H), 2.11 (s, 3H), 2.07 (s, 3H), 1.96 (s, 6H), 1.82 (s, 3H).

### Example 274

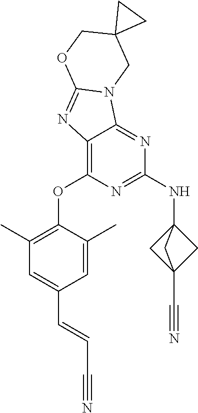 [SMILES: Cc1cc(/C=C/C#N)cc(C)c1Oc1nc(NC23CC(C#N)(C2)C3)nc2c1nc1n2CC2(CC2)CO1]

(E)-3-((4′-(4-(2-cyanovinyl)-2,6-dimethylphenoxy)-7′H,9′H-spiro[cyclopropane-1,8′-[1,3]oxazino[3,2-e]purin]-2′-yl)amino)bicyclo[1.1.1]pentane-1-carbonitrile (Compound 274) was prepared using the procedure described for the synthesis of (E)-3-((4-(4-(2-cyanovinyl)-2,6-dimethylphenoxy)-7,8-dihydro-6H-[1,3]oxazino[2,3-f]purin-2-yl)amino)bicyclo[1.1.1]pentane-1-carbonitrile (Compound 182) with the following modifications: 1,1-bis(bromomethyl)cyclopropane was used in place of 1,3-Diiodopropane. MS (m/z) 479.9 [M+H]+. ¹H NMR (400 MHz, DMSO-d6) δ 7.71 (s, 1H), 7.61 (d, J=16.7 Hz, 1H), 7.52 (s, 2H), 6.41 (d, J=16.7 Hz, 1H), 4.30 (s, 2H), 3.88 (s, 2H), 2.06 (s, 12H), 0.82 (d, J=3.2 Hz, 4H).

### Example 275

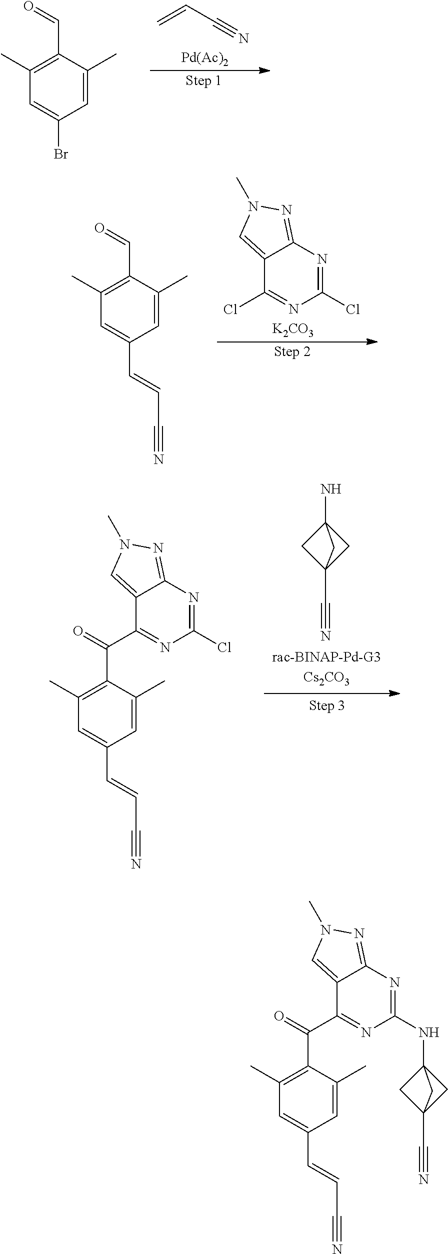

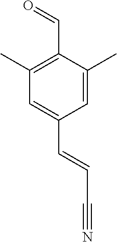

### (E)-3-(4-formyl-3,5-dimethylphenyl)acrylonitrile (Intermediate 275a)

A mixture of 4-bromo-2,6-dimethylbenzaldehyde (15.0 g, 70.4 mmol), acrylonitrile (37.3 g, 0.7 mol), Pd(OAc)₂ (1.5 g, 6.6 mmol), Na₂CO₃ (14.6 g, 140.8 mmol) and P(o-Tol)₃ (10.7 g, 35.2 mmol) in DMAC (150 mL) was stirred under N₂ at 140° C. overnight. To the reaction mixture was added water (100 mL) and the mixture was extracted with ethyl acetate (150 mL×3). The combined organic layer was washed with water (100 mL×2) and brine (100 mL), dried over Na₂SO₄ and concentrated in vacuo. The residue was subjected to silica gel column chromatography to give the title compound. MS (m/z) 186.30 [M+H]+.

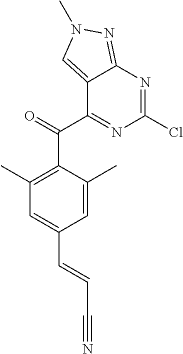 [SMILES: Cc1cc(/C=C/C#N)cc(C)c1C(=O)c1nc(Cl)nc2nn(C)cc12]

### (E)-3-(4-(6-chloro-2-methyl-2H-pyrazolo[3,4-d]pyrimidine-4-carbonyl)-3,5-dimethylphenyl)acrylonitrile (Intermediate 275b)

NaH (189 mg, 4.93 mmol, 60% dispersion in oil) was added to a solution of 4,6-dichloro-2-methyl-pyrazolo[3,4-d]pyrimidine (500 mg, 2.46 mmol), Intermediate 275a (547 mg, 2.96 mmol), and 1,3-dimethylimidazolium iodide (276 mg, 1.23 mmol) in DMSO (24 mL). The solution was then heated to 40° C. Upon reaction completion as judged by LCMS, saturated aqueous sodium bicarbonate was added to the mixture and the resulting solid was filtered off and dried on a lyophilizer. The resulting solid was purified via silica gel flash column chromatography (0-100% EtOAc/DCM) to afford the title compound. MS (m/z) 352.10, 354.09 [M+H]+ (Cl-isotopes).

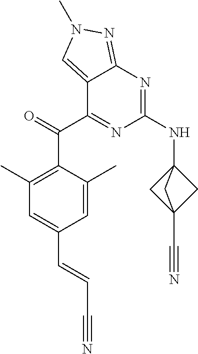 [SMILES: Cc1cc(/C=C/C#N)cc(C)c1C(=O)c1nc(NC23CC(C#N)(C2)C3)nc2nn(C)cc12]

### (E)-3-((4-(4-(2-cyanovinyl)-2,6-dimethylbenzoyl)-2-methyl-2H-pyrazolo[3,4-d]pyrimidin-6-yl)amino)bicyclo[1.1.1]pentane-1-carbonitrile (Compound 275)

Intermediate 275b (410 mg, 1.17 mmol), 3-aminobicyclo[1.1.1]pentane-1-carbonitrile (378 mg, 3.50 mmol), rac-BINAP-Pd-G3 (1159 mg, 1.17 mmol), and Cs₂CO₃ (1899 mg, 5.83 mmol) were suspended in 1,4-dioxane (12 mL). The reaction mixture was sparged with Ar for 1 min, then heated to 60° C. for 48 hours after which the mixture was allowed to cool to room temperature. The mixture was filtered over a silica plug using EtOAc and MeCN as eluting agents. The filtrate was purified via successive silica gel flash column chromatography (0-20% MeOH/DCM) and preparative HPLC (0-100% MeCN in water, 0.1% TFA) then lyophilized to afford the title compound as the trifluoroacetic acid salt. MS (m/z) 424.23 [M+H]+. ¹H NMR (400 MHz, DMSO-d6) δ 8.83 (s, 1H), 8.25 (s, 1H), 7.65 (d, J=16.7 Hz, 1H), 7.49 (s, 2H), 6.53 (d, J=16.7 Hz, 1H), 4.15 (s, 3H), 2.08 (s, 6H), 2.08 (6H, br s).

### Example 276

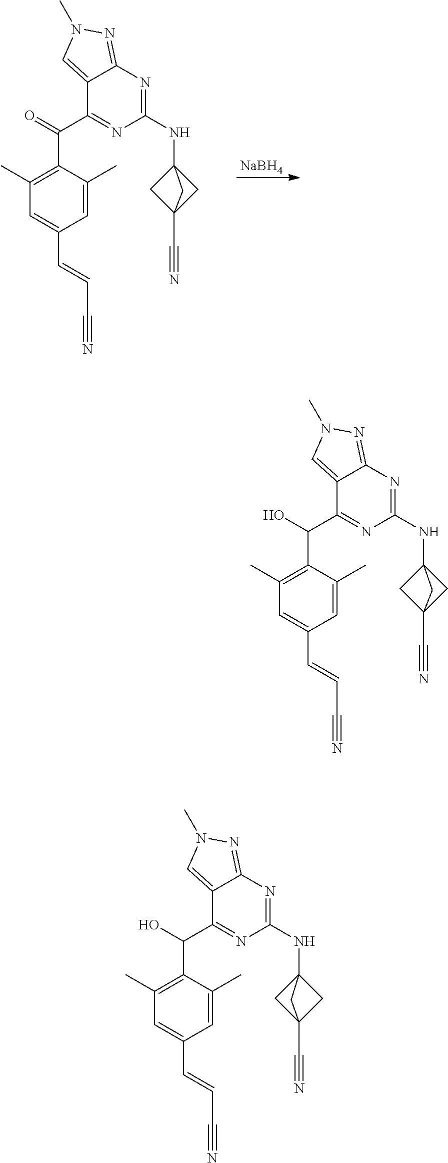 [SMILES: Cc1cc(/C=C/C#N)cc(C)c1C(=O)c1nc(NC23CC(C#N)(C2)C3)nc2nn(C)cc12 ; "NaBH4" ; Cc1cc(/C=C/C#N)cc(C)c1C(O)c1nc(NC23CC(C#N)(C2)C3)nc2nn(C)cc12 ; Cc1cc(/C=C/C#N)cc(C)c1C(O)c1nc(NC23CC(C#N)(C2)C3)nc2nn(C)cc12]

### (E)-3-((4-((4-(2-cyanovinyl)-2,6-dimethylphenyl)(hydroxy)methyl)-2-methyl-2H-pyrazolo[3,4-d]pyrimidin-6-yl)amino)bicyclo[1.1.1]pentane-1-carbonitrile (Compound 276)

To a suspension of Compound 275 (25 mg, 0.059 mmol) in methanol (0.5 mL) was added NaBH₄ (5.4 mg, 0.14 mmol). After 5 minutes, to the mixture was added brine and the mixture was extracted with EtOAc. The organic layer was concentrated and purified via preparative HPLC (0-100% MeCN in water, 0.1% TFA) then lyophilized to afford the title compound as the trifluoroacetic acid salt. MS (m/z) 426.23 [M+H]+. ¹H NMR (400 MHz, DMSO-d6) δ 8.52 (s, 1H), 7.72 (s, 1H), 7.55 (d, J=16.7 Hz, 1H), 7.32 (s, 2H), 6.40 (d, J=16.7 Hz, 1H), 6.22 (s, 1H), 4.05 (s, 3H), 2.23 (s, 6H), 2.23 (br s, 6H)

### Example 277

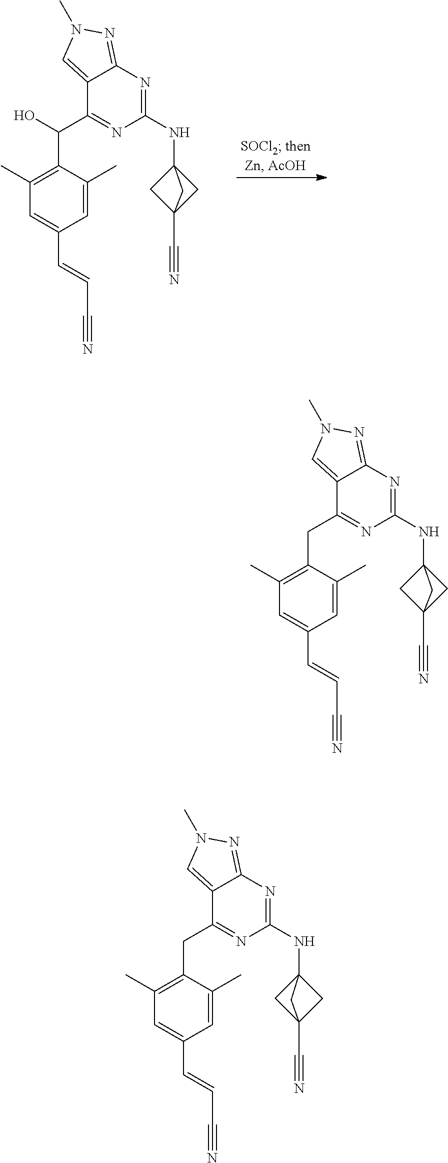 [SMILES: Cc1cc(/C=C/C#N)cc(C)c1C(O)c1nc(NC23CC(C#N)(C2)C3)nc2nn(C)cc12 ; "SOCl2; thenZn, AcOH" ; Cc1cc(/C=C/C#N)cc(C)c1Cc1nc(NC23CC(C#N)(C2)C3)nc2nn(C)cc12 ; Cc1cc(/C=C/C#N)cc(C)c1Cc1nc(NC23CC(C#N)(C2)C3)nc2nn(C)cc12]

(E)-3-((4-(4-(2-cyanovinyl)-2,6-dimethylbenzyl)-2-methyl-2H-pyrazolo[3,4-d]pyrimidin-6-yl)amino)bicyclo[1.1.1]pentane-1-carbonitrile (Compound 277):

To a solution of Compound 276 (50.0 mg, 0.112 mmol) in DCM (2 mL) was added SOCl₂ (50 μL, 0.69 mmol). The solution was stirred at 45° C. for 1 hour, then 60° C. for 30 minutes before being cooled to room temperature. The mixture was directly concentrated in vacuo. To the crude residue was added AcOH (3 mL) and Zn (73 mg, 1.1 mmol). The solution was heated at 70° C. for three hours then allowed to cool to room temperature. Et₂O and hexanes were added to the mixture and the solids were filtered off. The solids were purified via preparative HPLC (0-100% MeCN in water, 0.1% TFA) then lyophilized to afford the title compound as the trifluoroacetic acid salt. MS (m/z) 410.33 [M+H]+. ¹H NMR (400 MHz, DMSO-d6) δ 7.85 (s, 1H), 7.56 (d, J=16.7 Hz, 1H), 7.38 (s, 2H), 6.39 (d, J=16.7 Hz, 1H), 4.33 (s, 2H), 4.00 (s, 3H), 2.25 (br s, 6H) 2.21 (s, 6H).

### Example 278

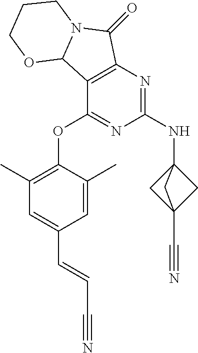 [SMILES: Cc1cc(/C=C/C#N)cc(C)c1Oc1nc(NC23CC(C#N)(C2)C3)nc2c1C1OCCCN1C2=O]

### (E)-3-((4-(4-(2-cyanovinyl)-2,6-dimethylphenoxy)-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 278)

To a solution of Intermediate 247d (100 mg, 0.219 mmol) in MeCN (1 mL) was added 2-aminoethanol (16 uL, 0.262 mmol) and the mixture was heated at 130° C. for 1 hour via microwave irradiation. The mixture was concentrated in vacuo and subjected to preparative HPLC (0-100% MeCN/H₂O, 0.1% TFA) and lyophilized to afford the title compound as the TFA salt. MS (m/z) 469.22 [M+H]+. ¹H NMR (400 MHz, DMSO-d6) δ 8.79 (s, 1H), 7.64 (d, J=16.7 Hz, 1H), 7.57 (s, 2H), 6.45 (d, J=16.7 Hz, 1H), 5.92 (s, 1H), 4.19 (dd, J=42.5, 12.5 Hz, 2H), 3.98 (m 1H), 3.32 (m, 1H), 2.09 (s, 3H), 2.06 (s, 3H), 1.98 (s, 6H), 1.69 (m, 2H).

### Examples 279 and 280

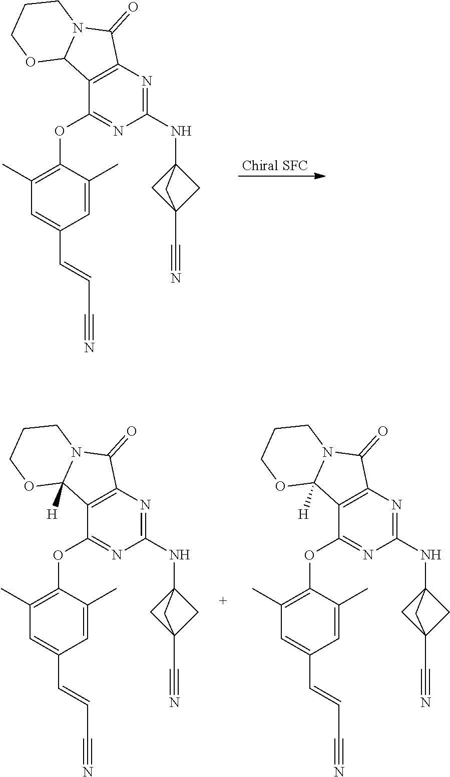 [SMILES: Cc1cc(/C=C/C#N)cc(C)c1Oc1nc(NC23CC(C#N)(C2)C3)nc2c1C1OCCCN1C2=O ; "Chiral SFC" ; Cc1cc(/C=C/C#N)cc(C)c1Oc1nc(NC23CC(C#N)(C2)C3)nc2c1[C@@H]1OCCCN1C2=O ; Cc1cc(/C=C/C#N)cc(C)c1Oc1nc(NC23CC(C#N)(C2)C3)nc2c1[C@H]1OCCCN1C2=O]

Compound 278 was subjected to chiral SFC (Column 1K, 4.6×100 mm, 5 mic, 50% EtOH/TFA cosolvent, 40° C. to yield the title compounds as their respective TFA-salts.

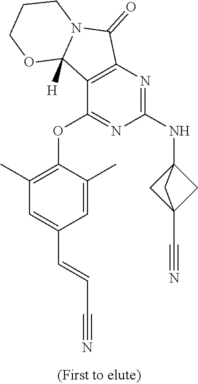 [SMILES: Cc1cc(/C=C/C#N)cc(C)c1Oc1nc(NC23CC(C#N)(C2)C3)nc2c1[C@@H]1OCCCN1C2=O]

### (S,E)-3-((4-(4-(2-cyanovinyl)-2,6-dimethylphenoxy)-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 279)

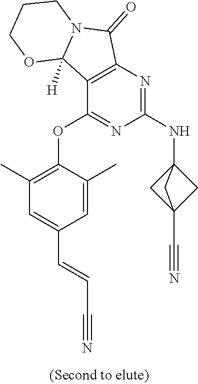 [SMILES: Cc1cc(/C=C/C#N)cc(C)c1Oc1nc(NC23CC(C#N)(C2)C3)nc2c1[C@H]1OCCCN1C2=O]

### (R,E)-3-((4-(4-(2-cyanovinyl)-2,6-dimethylphenoxy)-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 280)

### Examples 281 and 282

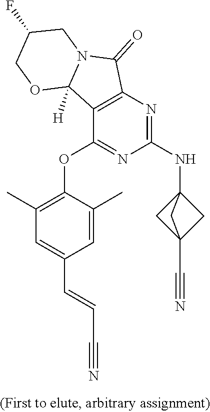 [SMILES: Cc1cc(/C=C/C#N)cc(C)c1Oc1nc(NC23CC(C#N)(C2)C3)nc2c1[C@H]1OC[C@H](F)CN1C2=O]

### 3-(((4bR,7R)-4-(4-((E)-2-cyanovinyl)-2,6-dimethylphenoxy)-7-fluoro-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 281)

Prepared by analogy to Compound 278, using (2R)-3-amino-2-fluoro-propan-1-ol in lieu of 2-aminoethanol. MS (m/z) 487.25 [M+H]+. ¹H NMR (400 MHz, DMSO-d6) δ 8.86 (s, 1H), 7.64 (d, J=16.7 Hz, 1H), 7.58 (s, 2H), 6.45 (d, J=16.7 Hz, 1H), 6.06 (s, 1H), 4.91 (d, J=45.7 Hz, 1H), 4.44-4.10 (m, 2H), 3.64 (ddd, J=41.1, 14.9, 2.1 Hz, 1H), 2.08 (s, 6H), 1.99 (s, 6H).

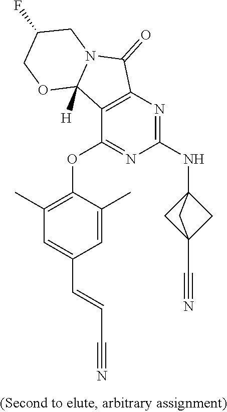 [SMILES: Cc1cc(/C=C/C#N)cc(C)c1Oc1nc(NC23CC(C#N)(C2)C3)nc2c1[C@@H]1OC[C@H](F)CN1C2=O]

### 3-(((4bS,7R)-4-(4-((E)-2-cyanovinyl)-2,6-dimethylphenoxy)-7-fluoro-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 282)

Prepared by analogy to Compound 278, using (2R)-3-amino-2-fluoro-propan-1-ol in lieu of 2-aminoethanol. MS (m/z) 487.20 [M+H]+. ¹H NMR (400 MHz, DMSO-d6) δ 8.87 (s, 1H), 7.64 (d, J=16.7 Hz, 1H), 7.57 (s, 2H), 6.45 (d, J=16.7 Hz, 1H), 6.03 (s, 1H), 4.77 (dtt, J=47.6, 10.0, 5.5 Hz, 1H), 4.56 (dd, J=12.3, 5.8 Hz, 1H), 4.33-4.26 (m, 1H), 3.92 (m, 1H) 3.36 (dt, J=12.2, 9.2 Hz, 1H), 2.11 (s, 3H), 2.05 (s, 3H), 1.98 (s, 6H).

### Examples 283 and 284

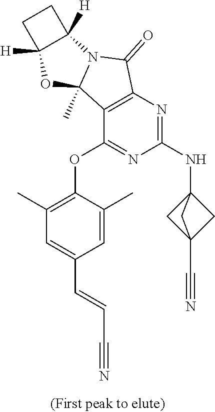 [SMILES: Cc1cc(/C=C/C#N)cc(C)c1Oc1nc(NC23CC(C#N)(C2)C3)nc2c1[C@@]1(C)O[C@@H]3CC[C@@H]3N1C2=O]

### 3-(((4bR,5aR,7aS)-4-(4-((E)-2-cyanovinyl)-2,6-dimethylphenoxy)-4b-methyl-9-oxo-4b,5a,6,7,7a,9-hexahydrocyclobuta[4′,5′]oxazolo[3′,2′:1,5]pyrrolo[3,4-d]pyrimidin-2-yl)amino)bicyclo[1.1.1]pentane-1-carbonitrile (Compound 283)

To a solution of methyl (E)-5-acetyl-2-((3-cyanobicyclo[1.1.1]pentan-1-yl)amino)-6-(4-(2-cyanovinyl)-2,6-dimethylphenoxy)pyrimidine-4-carboxylate (Intermediate 136d) (30 mg, 0.066 mmol) in MeCN (0.5 mL) was added (1S,2R)-2-aminocyclobutan-1-ol hydrochloride (46 mg, 0.66 mmol). The solution was heated to 130° C. via microwave irradiation for 3 hours. At this point, additional MeCN (1 mL) was added and the solution was heated another hour at 130° C. The mixture was cooled to room temp and additional (1S,2R)-2-aminocyclobutan-1-ol hydrochloride (100 mg, 1.4 mmol) was added and the mixture was heated for 9 hours at 130° C. via microwave irradiation then 8 hours at 150° C. The mixture was then concentrated in vacuo and subjected to preparative HPLC (0-100% MeCN/H₂O, 0.1% TFA) and lyophilized to afford the title compound and Compound 284 as TFA salts. The stereochemistry was arbitrarily assigned. MS (m/z) 493.08 [M−H]−. ¹H NMR (400 MHz, DMSO-d6) δ 8.84 (s, 1H), 7.71-7.54 (m, 3H), 6.45 (d, J=16.6 Hz, 1H), 5.10 (m, 1H), 4.37 (m, 1H), 2.26-1.97 (m, 14H), 1.74 (s, 3H).

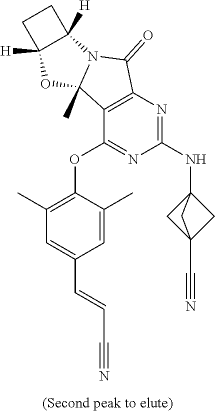 [SMILES: Cc1cc(/C=C/C#N)cc(C)c1Oc1nc(NC23CC(C#N)(C2)C3)nc2c1[C@]1(C)O[C@@H]3CC[C@@H]3N1C2=O]

3-(((4bS,5aR,7aS)-4-(4-((E)-2-cyanovinyl)-2,6-dimethylphenoxy)-4b-methyl-9-oxo-4b,5a,6,7,7a,9-hexahydrocyclobuta[4′,5′]oxazolo[3′,2′:1,5]pyrrolo[3,4-d]pyrimidin-2-yl)amino)bicyclo[1.1.1]pentane-1-carbonitrile (Compound 284): The stereochemistry was arbitrarily assigned. MS (m/z) 493.11 [M−H]−. ¹H NMR (400 MHz, DMSO-d6) δ 8.81 (s, 1H), 7.64 (d, J=16.6 Hz, 1H), 7.59 (s, 2H), 6.45 (d, J=16.7 Hz, 1H), 5.12 (m, 1H), 4.83-4.73 (m, 1H), 2.22-1.93 (m, 19H).

### Example 285

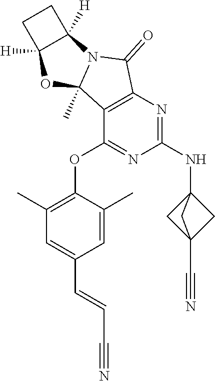 [SMILES: Cc1cc(/C=C/C#N)cc(C)c1Oc1nc(NC23CC(C#N)(C2)C3)nc2c1[C@@]1(C)O[C@H]3CC[C@H]3N1C2=O]

### 3-(((4bR,5aS,7aR)-4-(4-((E)-2-cyanovinyl)-2,6-dimethylphenoxy)-4b-methyl-9-oxo-4b,5a,6,7,7a,9-hexahydrocyclobuta[4′,5′]oxazolo[3′,2′:1,5]pyrrolo[3,4-d]pyrimidin-2-yl)amino)bicyclo[1.1.1]pentane-1-carbonitrile (Compound 285)

Prepared by analogy to Compound 283, using (1R,2S)-2-aminocyclobutan-1-ol hydrochloride in lieu of (1S,2R)-2-aminocyclobutan-1-ol hydrochloride. The stereochemistry was arbitrarily assigned. MS (m/z) 495.28 [M+H]+. ¹H NMR (400 MHz, DMSO-d6) δ 8.81 (s, 1H), 7.70-7.53 (m, 3H), 6.45 (d, J=16.8 Hz, 1H), 5.13 (m, 1H), 4.76 (m, 1H), 2.18-2.05 (m, 10H), 1.96 (s, 6H).

### Example 286

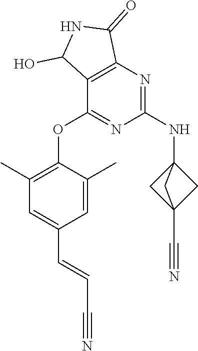 [SMILES: Cc1cc(/C=C/C#N)cc(C)c1Oc1nc(NC23CC(C#N)(C2)C3)nc2c1C(O)NC2=O]

### (E)-3-((4-(4-(2-cyanovinyl)-2,6-dimethylphenoxy)-5-hydroxy-7-oxo-6,7-dihydro-5H-pyrrolo[3,4-d]pyrimidin-2-yl)amino)bicyclo[1.1.1]pentane-1-carbonitrile (Compound 286)

To a solution of ethyl (E)-2-((3-cyanobicyclo[1.1.1]pentan-1-yl)amino)-6-(4-(2-cyanovinyl)-2,6-dimethylphenoxy)-5-formylpyrimidine-4-carboxylate (Intermediate 247d) (50 mg, 0.11 mmol) in EtOH (1 mL) was added aqueous ammonium hydroxide (10 drops). After 10 minutes, the mixture was concentrated in vacuo and subjected to preparative HPLC (0-100% MeCN/H₂O, 0.1% TFA) and lyophilized to afford the title product. MS (m/z) 429.15 [M+H]+. ¹H NMR (400 MHz, DMSO-d6) δ 9.39 (s, 1H), 8.58 (s, 1H), 7.62 (d, J=16.7 Hz, 1H), 7.54 (d, J=5.4 Hz, 2H), 6.42 (d, J=16.7 Hz, 1H), 6.02 (s, 1H), 2.09 (s, 6H), 1.97 (s, 6H).

### Example 287

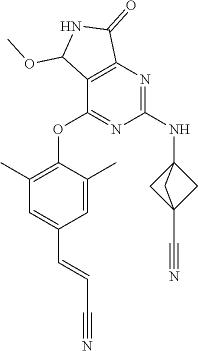 [SMILES: COC1NC(=O)c2nc(NC34CC(C#N)(C3)C4)nc(Oc3c(C)cc(/C=C/C#N)cc3C)c21]

### (E)-3-((4-(4-(2-cyanovinyl)-2,6-dimethylphenoxy)-5-methoxy-7-oxo-6,7-dihydro-5H-pyrrolo[3,4-d]pyrimidin-2-yl)amino)bicyclo[1.1.1]pentane-1-carbonitrile (Compound 287)

To a solution of Compound 286 (10 mg, 0.023 mmol) in MeOH (0.5 mL) was added TFA (3 drops). The solution was stirred at room temperature for 5 minutes then heated to 40° C. for 3 hours. To the mixture was added MeCN and H₂O and the mixture was directly lyophilized to afford the title product as the TFA salt. MS (m/z) 443.22 [M+H]+. ¹H NMR (400 MHz, DMSO-d6) δ 9.59 (s, 1H), 8.75 (s, 1H), 7.64 (d, J=16.6 Hz, 1H), 7.56 (d, J=10.3 Hz, 2H), 6.45 (d, J=16.7 Hz, 1H), 6.08 (s, 1H), 3.24 (s, 3H), 2.09 (s, 6H), 1.98 (s, 6H).

### Example 288

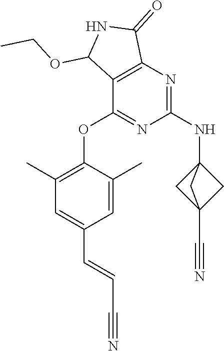 [SMILES: CCOC1NC(=O)c2nc(NC34CC(C#N)(C3)C4)nc(Oc3c(C)cc(/C=C/C#N)cc3C)c21]

### (E)-3-((4-(4-(2-cyanovinyl)-2,6-dimethylphenoxy)-5-ethoxy-7-oxo-6,7-dihydro-5H-pyrrolo[3,4-d]pyrimidin-2-yl)amino)bicyclo[1.1.1]pentane-1-carbonitrile (Compound 288)

Prepared by analogy to Compound 287, using EtOH in lieu of MeOH and adding THE (0.5 mL) and heating at 50° C. for 6 hours. MS (m/z) 457.26 [M+H]+. ¹H NMR (400 MHz, DMSO-d6) δ 9.61 (s, 1H), 8.73 (s, 1H), 7.64 (d, J=16.7 Hz, 1H), 7.57 (s, 2H), 6.44 (d, J=16.7 Hz, 1H), 6.08 (s, 1H), 3.59-3.48 (m, 2H), 2.12-2.05 (m, 6H), 1.97 (s, 6H), 1.16 (t, J=7.0 Hz, 3H).

### Example 289

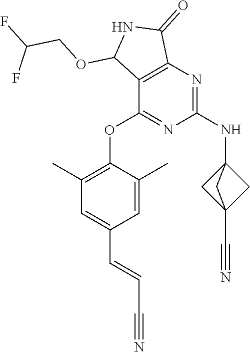 [SMILES: Cc1cc(/C=C/C#N)cc(C)c1Oc1nc(NC23CC(C#N)(C2)C3)nc2c1C(OCC(F)F)NC2=O]

### (E)-3-((4-(4-(2-cyanovinyl)-2,6-dimethylphenoxy)-5-(2,2-difluoroethoxy)-7-oxo-6,7-dihydro-5H-pyrrolo[3,4-d]pyrimidin-2-yl)amino)bicyclo[1.1.1]pentane-1-carbonitrile (Compound 289)

Prepared by analogy to Compound 287, using 2-difluoropropanol in lieu of MeOH and stirring at room temperature. MS (m/z) 493.26 [M+H]+. ¹H NMR (400 MHz, DMSO-d6) δ 9.76-9.59 (m, 1H), 8.79 (s, 1H), 7.63 (d, J=16.8 Hz, 1H), 7.57 (s, 2H), 6.44 (d, J=16.7 Hz, 1H), 6.38-6.02 (m, 2H), 3.90-3.63 (m, 2H), 2.08 (q, J=2.4 Hz, 6H), 1.97 (s, 6H).

### Example 290

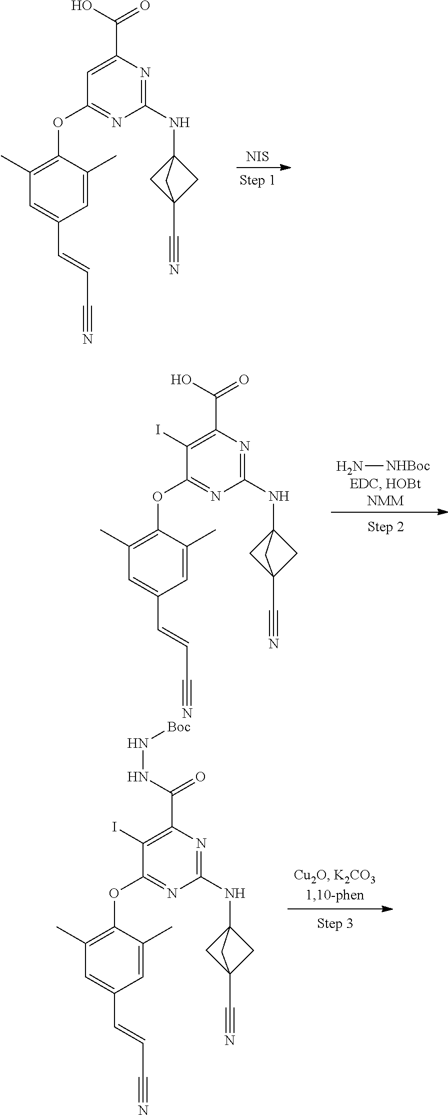

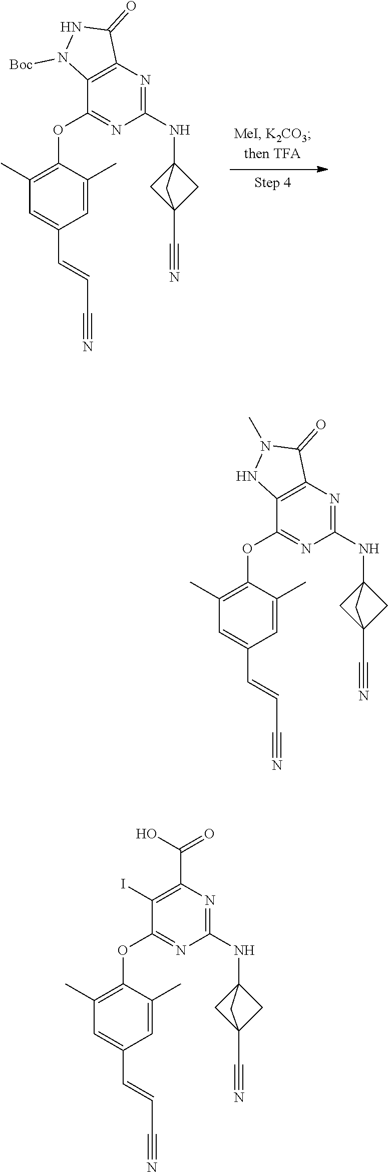 [SMILES: Cc1cc(/C=C/C#N)cc(C)c1Oc1nc(NC23CC(C#N)(C2)C3)nc2c(=O)[nH]n(C(=O)OC(C)(C)C)c12 ; Cc1cc(/C=C/C#N)cc(C)c1Oc1nc(NC23CC(C#N)(C2)C3)nc2c(=O)n(C)[nH]c12 ; Cc1cc(/C=C/C#N)cc(C)c1Oc1nc(NC23CC(C#N)(C2)C3)nc(C(=O)O)c1I]

### (E)-2-((3-cyanobicyclo[1.1.1]pentan-1-yl)amino)-6-(4-(2-cyanovinyl)-2,6-dimethylphenoxy)-5-iodopyrimidine-4-carboxylic acid (Intermediate 290a)

(E)-2-((3-cyanobicyclo[1.1.1]pentan-1-yl)amino)-6-(4-(2-cyanovinyl)-2,6-dimethylphenoxy)pyrimidine-4-carboxylic acid (5.00 g, 12.5 mmol) and NIS (3.08 g, 13.7 mmol) were suspended in AcOH (50 mL). After stirring at room temperature for 45 minutes, another portion of NIS (3.08 g, 13.7 mmol) was added and the mixture was stirred for another 2 hours. The mixture was then concentrated in vacuo and Et₂O and hexanes were added to the residue. The resulting solid was filtered off, washing with hexanes, to afford the title compound. MS (m/z) 528.05 [M+H]+.

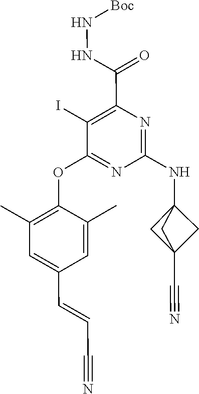 [SMILES: Cc1cc(/C=C/C#N)cc(C)c1Oc1nc(NC23CC(C#N)(C2)C3)nc(C(=O)NNC(=O)OC(C)(C)C)c1I]

### tert-butyl (E)-2-(2-((3-cyanobicyclo[1.1.1]pentan-1-yl)amino)-6-(4-(2-cyanovinyl)-2,6-dimethylphenoxy)-5-iodopyrimidine-4-carbonyl)hydrazine-1-carboxylate (Intermediate 290b)

To a solution of Intermediate 290a (250 mg, 0.474 mmol), EDC (182 mg, 0.948 mmol), HOBt (141 mg, 1.04 mmol), and tert-butyl carbamate (94.0 mg, 0.711 mmol) in 1,4-dioxane (0.5 mL) was added N-methylmorpholine (48 mg, 0.47 mmol). The reaction was stirred a room temperature until conversion was deemed complete by LCMS then the mixture was added to a solution of saturated sodium bicarbonate. The mixture was extracted with EtOAc, the combined organic layers were dried with Na₂SO₄ and filtered. The filtrate was concentrated in vacuo and subjected to silica gel flash column chromatography (0-100% DCM/EtOAc) to afford the title compound. MS (m/z) 641.47 [M+H]+.

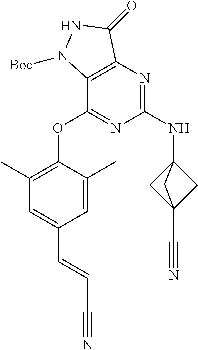 [SMILES: Cc1cc(/C=C/C#N)cc(C)c1Oc1nc(NC23CC(C#N)(C2)C3)nc2c(=O)[nH]n(C(=O)OC(C)(C)C)c12]

### tert-butyl (E)-5-((3-cyanobicyclo[1.1.1]pentan-1-yl)amino)-7-(4-(2-cyanovinyl)-2,6-dimethylphenoxy)-3-oxo-2,3-dihydro-1H-pyrazolo[4,3-d]pyrimidine-1-carboxylate (Intermediate 290c)

Intermediate 290b (843 mg, 1.31 mmol), potassium carbonate (363 mg, 2.63 mmol), Cu₂O (38 mg, 0.26 mmol), and 1,10-phenanthroline (36 mg, 0.20 mmol) were suspended in DMF (9 mL). The vessel containing the mixture was subjected to vacuum then backfilled with Ar three times then sealed. The mixture was heated to 80° C. for approximately 16 hours then cooled to room temperature and added to a saturated aqueous sodium bicarbonate solution. The mixture was extracted with EtOAc and the combined organic layers were dried with sodium sulfate, filtered and concentrated in vacuo. The resulting residue was subjected to silica gel flash column chromatography (0-100% EtOAc/DCM then 0-25% MeOH/EtOAc) to afford the title compound. MS (m/z) 513.95 [M+H]+.

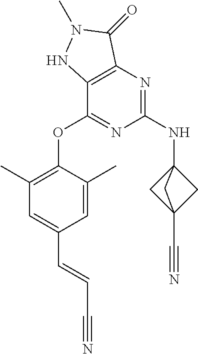 [SMILES: Cc1cc(/C=C/C#N)cc(C)c1Oc1nc(NC23CC(C#N)(C2)C3)nc2c(=O)n(C)[nH]c12]

### (E)-3-((7-(4-(2-cyanovinyl)-2,6-dimethylphenoxy)-2-methyl-3-oxo-2,3-dihydro-1H-pyrazolo[4,3-d]pyrimidin-5-yl)amino)bicyclo[1.1.1]pentane-1-carbonitrile (Compound 290)

To a solution of Intermediate 290c (40 mg, 0.078 mmol) and potassium carbonate (32 mg, 0.23 mmol) in DMF (0.5 mL) was added iodomethane (three drops). The solution was stirred at room temperature for approximately an hour. To the solution was added TFA (1 mL) and the solution was stirred at room temperature overnight. The mixture was filtered and the filtrated was subjected to preparative HPLC (0-100% MeCN/H₂O, 0.1% TFA) and lyophilized to afford the title product. MS (m/z) 426.14 [M−H]−. ¹H NMR (400 MHz, Acetonitrile-d3) δ 7.49 (d, J=16.7 Hz, 1H), 7.43 (s, 2H), 6.09 (d, J=16.7 Hz, 1H), 2.14 (s, 3H), 2.08 (s, 6H), 2.08 (br s, 6H).

### Example 291

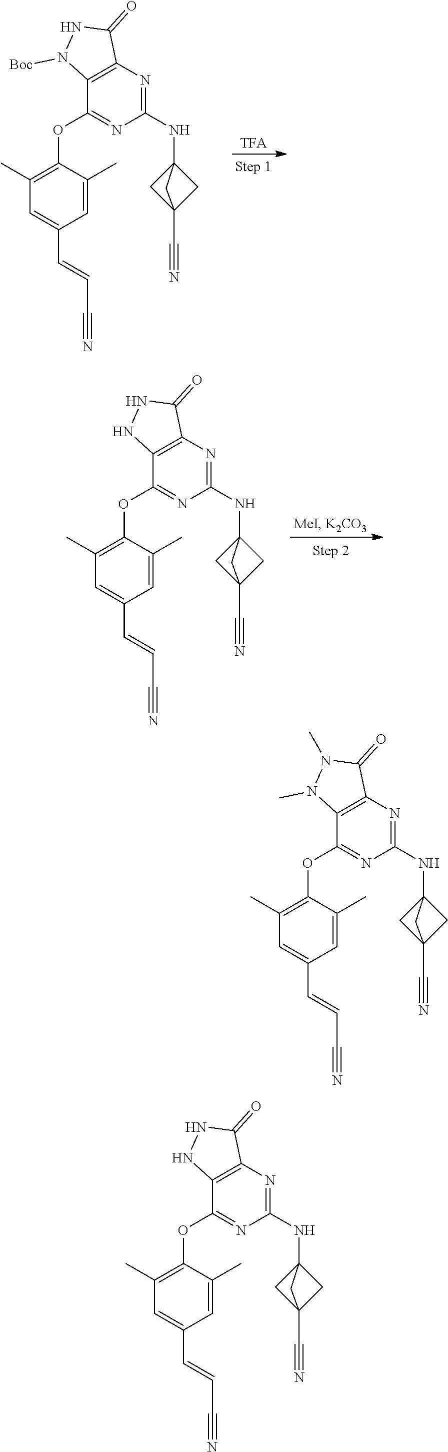

### (E)-3-((7-(4-(2-cyanovinyl)-2,6-dimethylphenoxy)-3-oxo-2,3-dihydro-1H-pyrazolo[4,3-d]pyrimidin-5-yl)amino)bicyclo[1.1.1]pentane-1-carbonitrile (Intermediate 291a)

Intermediate 290c (100 mg, 0.195 mmol) was suspended in TFA (0.5 mL) and stirred at room temperature. After 30 minutes the mixture was concentrated in vacuo and the intermediate was used as is. MS (m/z) 414.21 [M+H]+.

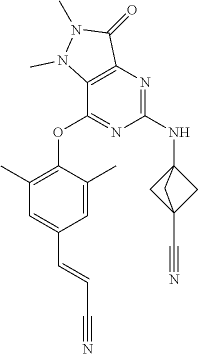 [SMILES: Cc1cc(/C=C/C#N)cc(C)c1Oc1nc(NC23CC(C#N)(C2)C3)nc2c(=O)n(C)n(C)c12]

### (E)-3-((7-(4-(2-cyanovinyl)-2,6-dimethylphenoxy)-1,2-dimethyl-3-oxo-2,3-dihydro-1H-pyrazolo[4,3-d]pyrimidin-5-yl)amino)bicyclo[1.1.1]pentane-1-carbonitrile (Compound 291)

Intermediate 291a (25.0 mg, 0.0605 mmol) and potassium carbonate (25.1 mg, 0.181 mmol) was suspended in NMP (0.5 mL). Iodomethane (a few drops) was added and the solution was stirred at room temperature over night, then filtered and subjected to preparative HPLC (0-100% MeCN/H₂O, 0.1% TFA) and lyophilized to afford the title product as the TFA salt. MIS (m/z) 442.31 [M+H]+. ¹H NMR (400 MHz, DMSO-d6) δ 7.74 (s, 1H), 7.63 (d, J=16.8 Hz, 1H), 7.56 (s, 2H), 6.45 (d, J=16.7 Hz, 1H), 4.04 (s, 3H), 4.00 (s, 3H), 2.12 (s, 12H).

### Example 292

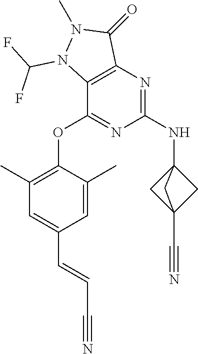 [SMILES: Cc1cc(/C=C/C#N)cc(C)c1Oc1nc(NC23CC(C#N)(C2)C3)nc2c(=O)n(C)n(C(F)F)c12]

### (E)-3-((7-(4-(2-cyanovinyl)-2,6-dimethylphenoxy)-1-(difluoromethyl)-2-methyl-3-oxo-2,3-dihydro-1H-pyrazolo[4,3-d]pyrimidin-5-yl)amino)bicyclo[1.1.1]pentane-1-carbonitrile (Compound 292)

To a solution of Intermediate 290c (45 mg, 0.088 mmol) and potassium carbonate (36 mg, 0.26 mmol) in DMF (0.5 mL) was added iodomethane (three drops). The solution was stirred at room temperature for approximately an hour. To the solution was added TFA (1 mL) and the solution was stirred at room temperature overnight. The mixture was diluted with DMF (1 mL) and potassium carbonate (1 g, 7 mmol) was added until effervescence ceased. Further diluted with DMF (1 mL) and iododifluoromethane (100 uL, 0.200 mmol) was added and the solution was heated at 60 C for 1 hour, 70 C for 16 hours, then 85 C for 2 hours. The mixture was cooled to room temperature, filtered, and subjected to preparative HPLC (0-100% MeCN/H₂O, 0.1% TFA) to afford the title compound and Compound 293 as their TFA salts. MS (m/z) 478.20 [M+H]+. ¹H NMR (400 MHz, Acetonitrile-d3) δ 7.69-7.35 (m, 4H), 6.25 (d, J=16.6 Hz, 1H), 3.63 (s, 3H), 2.18 (m, 12H).

### Example 293

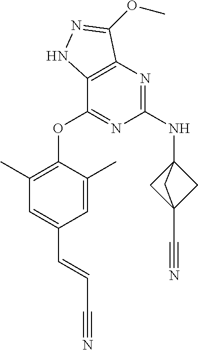 [SMILES: COc1n[nH]c2c(Oc3c(C)cc(/C=C/C#N)cc3C)nc(NC34CC(C#N)(C3)C4)nc12]

(E)-3-((7-(4-(2-cyanovinyl)-2,6-dimethylphenoxy)-3-methoxy-1H-pyrazolo[4,3-d]pyrimidin-5-yl)amino)bicyclo[1.1.1]pentane-1-carbonitrile: MS (m/z) 428.25 [M+H]+. ¹H NMR (400 MHz, CD30D) δ 7.53 (d, J=19.5 Hz, 3H), 6.25 (d, J=16.6 Hz, 1H), 4.11 (s, 3H), 2.19 (s, 6H), 2.13 (s, 6H).

### Example 294

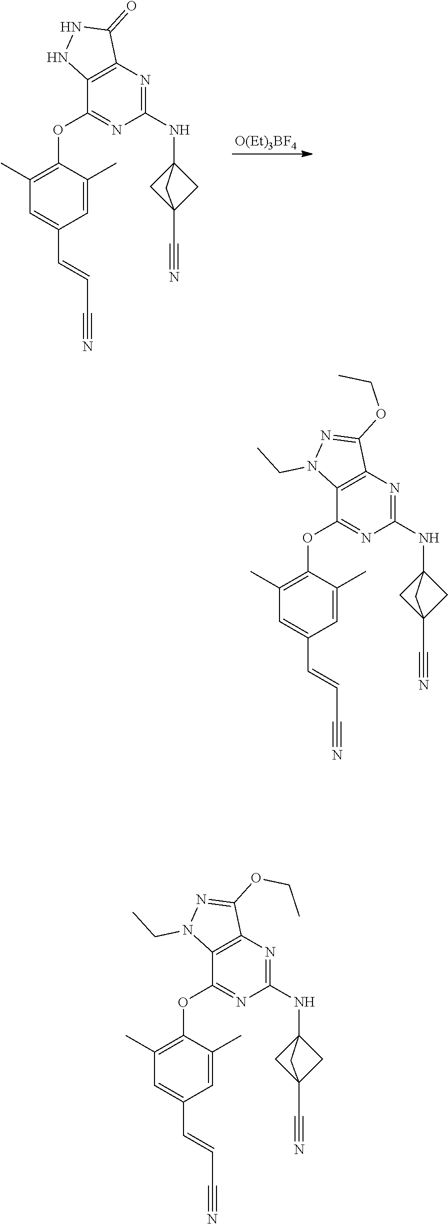 [SMILES: Cc1cc(/C=C/C#N)cc(C)c1Oc1nc(NC23CC(C#N)(C2)C3)nc2c(=O)[nH][nH]c12 ; CCOc1nn(CC)c2c(Oc3c(C)cc(/C=C/C#N)cc3C)nc(NC34CC(C#N)(C3)C4)nc12 ; "O(Et)3BF4" ; CCOc1nn(CC)c2c(Oc3c(C)cc(/C=C/C#N)cc3C)nc(NC34CC(C#N)(C3)C4)nc12]

### (E)-3-((7-(4-(2-cyanovinyl)-2,6-dimethylphenoxy)-3-ethoxy-1-ethyl-1H-pyrazolo[4,3-d]pyrimidin-5-yl)amino)bicyclo[11.1.1]pentane-1-carbonitrile (Compound 294)

Intermediate 291a (100 mg, 0.195 mmol) was suspended in DCE (2 mL) and triethyloxonium tetrafluoroborate (74 mg, 0.39 mmol) was added to the solution at 0° C. After stirring at this temperature for 1 hour, the mixture was warmed to room temperature and stirred at this temperature until the Intermediate 291a was fully consumed as indicated by LCMS. The mixture was concentrated in vacuo and the residue was subjected to preparative HPLC (0-100% MeCN/H₂O, 0.1% TFA) to afford the title compound as the TFA-salt. MS (m/z) 470.12 [M+H]+. ¹H NMR (400 MHz, DMSO-d6) δ 8.18 (s, 1H), 7.64 (d, J=16.7 Hz, 1H), 7.58 (s, 2H), 6.46 (d, J=16.7 Hz, 1H), 3.99 (q, J=6.9 Hz, 2H), 3.88 (q, J=7.0 Hz, 2H), 2.11 (m, 12H), 1.19 (t, J=7.1 Hz, 3H), 0.79 (t, J=6.9 Hz, 3H).

### Example 295

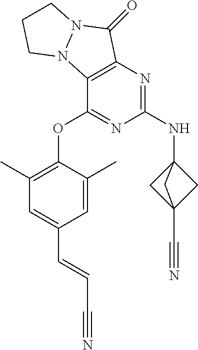 [SMILES: Cc1cc(/C=C/C#N)cc(C)c1Oc1nc(NC23CC(C#N)(C2)C3)nc2c(=O)n3n(c12)CCC3]

### (E)-3-((4-(4-(2-cyanovinyl)-2,6-dimethylphenoxy)-10-oxo-7,8-dihydro-6H,10H-pyrazolo[1′,2′:1,2]pyrazolo[4,3-d]pyrimidin-2-yl)amino)bicyclo[1.1.1]pentane-1-carbonitrile (Compound 295)

Prepared by analogy to Compound 291, using 1,3-diiodopropane in lieu of iodomethane. MS (m/z) 454.31 [M+H]+. ¹H NMR (400 MHz, DMSO-d6) δ 8.16 (s, 1H), 7.63 (d, J=16.7 Hz, 1H), 7.57 (s, 2H), 6.45 (d, J=16.7 Hz, 1H), 3.79 (t, J=7.2 Hz, 2H), 2.67-2.57 (m, 2H), 2.10 (m, 14H).

### Example 296

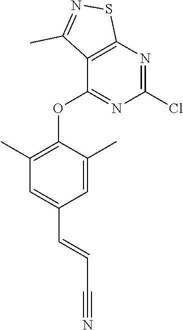 [SMILES: Cc1cc(/C=C/C#N)cc(C)c1Oc1nc(Cl)nc2snc(C)c12]

### (E)-3-(4-((6-chloro-3-methylisothiazolo[5,4-d]pyrimidin-4-yl)oxy)-3,5-dimethylphenyl)acrylonitrile (Intermediate 296a)

4,6-dichloro-3-methylisothiazolo[5,4-d]pyrimidine(1.00 g, 4.54 mmol), (E)-3-(4-hydroxy-3,5-dimethyl-phenyl)prop-2-enenitrile (826 mg, 4.77 mmol), and K₂CO₃ (3.14 g, 22.7 mmol) were suspended in DMF (25 mL) and stirred at room temperature for 15 minutes. To the mixture was added water and the solids were filtered off. The solids were dissolved in DCM and dried with Na₂SO₄. The mixture was filtered and the filtrate was concentrated in vacuo. The crude material was used directly in the next transformation.

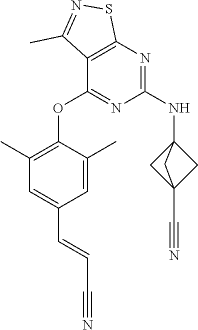 [SMILES: Cc1cc(/C=C/C#N)cc(C)c1Oc1nc(NC23CC(C#N)(C2)C3)nc2snc(C)c12]

### (E)-3-((4-(4-(2-cyanovinyl)-2,6-dimethylphenoxy)-3-methylisothiazolo[5,4-d]pyrimidin-6-yl)amino)bicyclo[1.1.1]pentane-1-carbonitrile (Compound 296)

Intermediate 296a (100 mg, 0.280 mmol), 3-aminobicyclo[1.1.1]pentane-1-carbonitrile; hydrochloride (81 mg, 0.56 mmol), K₂CO₃ (194 mg 1.40 mmol) were suspended in NMP (1.5 mL) and heated at 60° C. for approximately 16 hours. The mixture was cooled to r.t., filtered and subjected to preparative HPLC (0-100% MeCN in water, 0.1% TFA) then lyophilized to afford the title compound as the trifluoroacetic acid salt. MS (m/z) 429.39 [M+H]+. ¹H NMR (400 MHz, DMSO-d6) δ 8.74 (s, 1H), 7.72-7.45 (m, 3H), 6.47 (d, J=16.6 Hz, 1H), 2.76 (s, 3H), 2.12 (s, 6H), 2.00 (s, 6H).

### Example 297

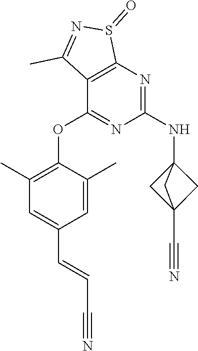 [SMILES: CC1=NS(=O)c2nc(NC34CC(C#N)(C3)C4)nc(Oc3c(C)cc(/C=C/C#N)cc3C)c21]

### (E)-3-((4-(4-(2-cyanovinyl)-2,6-dimethylphenoxy)-3-methyl-1-oxidoisothiazolo[5,4-d]pyrimidin-6-yl)amino)bicyclo[1.1.1]pentane-1-carbonitrile (Compound 297)

Compound 296 (100 mg, 0.233 mmol) and mCPBA (288 mg, 1.17 mmol) were combined in NMP (1.5 mL) and the mixture was heated at 75° C. for 1.5 hours. Another portion of mCPBA (288 mg, 1.17 mmol) was added and the mixture was returned to heating. After an additional 1.5 hours another portion of mCPBA (288 mg, 1.17 mmol) was added and the mixture was heated for 2 more hours before being cooled to room temperature. The mixture was filtered and the filtrate was subjected to preparative HPLC (0-100% MeCN/H₂O, 0.1% TFA) and lyophilized to afford the title compound. MS (m/z) 445.09 [M+H]+. ¹H NMR (400 MHz, DMSO-d6) δ 9.41 (s, 1H), 7.65 (d, J=16.7 Hz, 1H), 7.60 (s, 2H), 6.47 (d, J=16.7 Hz, 1H), 2.78 (s, 3H), 2.15 (s, 6H), 2.04 (s, 6H).

### Example 298

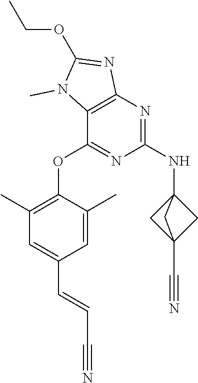 [SMILES: CCOc1nc2nc(NC34CC(C#N)(C3)C4)nc(Oc3c(C)cc(/C=C/C#N)cc3C)c2n1C]

### (E)-3-((6-(4-(2-cyanovinyl)-2,6-dimethylphenoxy)-8-ethoxy-7-methyl-7H-purin-2-yl)amino)bicyclo[1.1.1]pentane-1-carbonitrile (Compound 298)

To a mixture of (E)-3-((6-(4-(2-cyanovinyl)-2,6-dimethylphenoxy)-7-methyl-8-oxo-8,9-dihydro-7H-purin-2-yl)amino)bicyclo[1.1.1]pentane-1-carbonitrile (Compound 24) (37.0 mg, 0.0866 mmol) in DCM (1.00 mL) was added triethyloxonium tetrafluoroborate (0.0164 g, 0.0866 mmol), and the mixture was stirred at rt overnight. The mixture was then concentrated in vacuo and subjected to preparative HPLC (0-100% MeCN/H₂O) to afford the title product. MS (m/z) 456.18 [M+H]+. ¹H NMR (400 MHz, DMSO-d6) δ 7.62 (d, J=16.7 Hz, 1H), 7.52 (d, J=4.4 Hz, 3H), 6.42 (d, J=16.7 Hz, 1H), 4.54 (q, J=7.0 Hz, 2H), 3.69 (s, 3H), 2.19-1.97 (m, 12H), 1.41 (t, J=7.1 Hz, 3H).

### Example 299

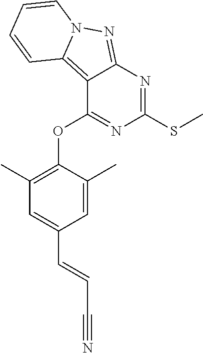

(E)-3-(3,5-dimethyl-4-((2-(methylthio)pyrido[1′,2′:1,5]pyrazolo[3,4-d]pyrimidin-4-yl)oxy)phenyl)acrylonitrile (Intermediate 299a) was prepared in a similar manner to Intermediate 79a, using 4-chloro-2-(methylthio)pyrido[1′,2′:1,5]pyrazolo[3,4-d]pyrimidine in lieu of 4-chloro-7,7-difluoro-2-(methylthio)-6,7-dihydro-5H-cyclopenta[d]pyrimidine. (i z) 384.17 [M+1]5.

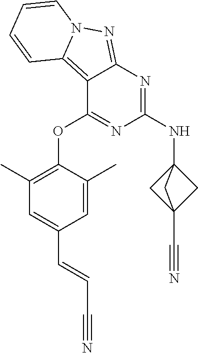 [SMILES: Cc1cc(/C=C/C#N)cc(C)c1Oc1nc(NC23CC(C#N)(C2)C3)nc2nn3ccccc3c12]

### (E)-3-((4-(4-(2-cyanovinyl)-2,6-dimethylphenoxy)pyrido[1′,2′:1,5]pyrazolo[3,4-d]pyrimidin-2-yl)amino)bicyclo[1.1.1]pentane-1-carbonitrile (Compound 299)

Prepared in a similar manner to Compound 79 using (E)-3-(3,5-dimethyl-4-((2-(methylthio)pyrido[1′,2′:1,5]pyrazolo[3,4-d]pyrimidin-4-yl)oxy)phenyl)acrylonitrile (190 mg, 0.490 mmol) in lieu of (E)-3-(4-((7,7-difluoro-2-(methylthio)-6,7-dihydro-5Hcyclopenta[d]pyrimidin-4-yl)oxy)-3,5-dimethylphenyl)acrylonitrile and using 3-aminobicyclo[1.1.1]pentane-1-carbonitrile; hydrochloride (106 mg, 0.735 mmol) and potassium carbonate (339 mg, 2.45 mmol). MS (m/z) 448.18 [M+H]+. ¹H NMR (400 MHz, DMSO-d6) δ 8.99 (d, J=6.8 Hz, 1H), 8.14 (d, J=8.6 Hz, 1H), 7.72 (t, J=7.9 Hz, 1H), 7.68-7.54 (m, 2H), 7.39 (t, J=7.0 Hz, 1H), 6.45 (d, J=16.7 Hz, 1H), 2.13 (s, 6H), 2.00 (br s, 6H).

### Example 300

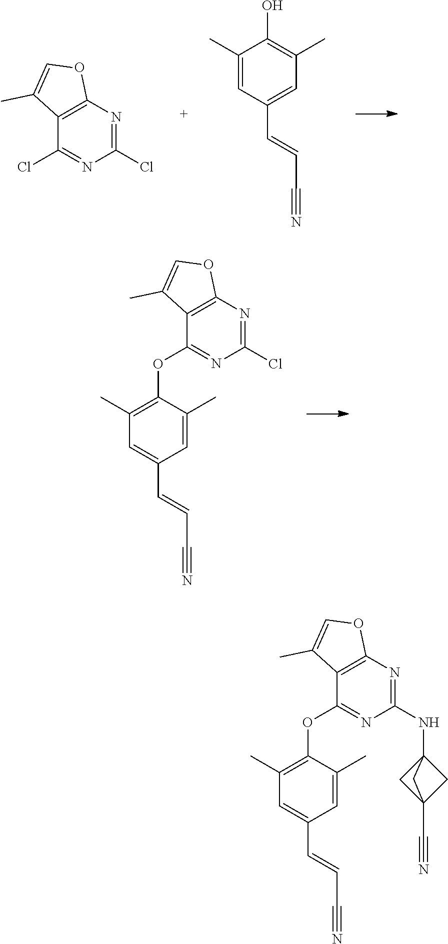

### Step 1, preparation of (E)-3-(4-((2-chloro-5-methylfuro[2,3-d]pyrimidin-4-yl)oxy)-3,5-dimethylphenyl)acrylonitrile (Intermediate 300a)

(E)-3-(4-hydroxy-3,5-dimethylphenyl)acrylonitrile (0.438 mmol, 76 mg), 2,4-dichloro-5-methylfuro[2,3-d]pyrimidine (0.438 mmol, 89 mg), and potassium carbonate (0.548 mmol, 76 mg) were mixed in DMF (1.1 mL). The mixture was stirred in a metal heating block at 60° C. for 8 h at which point water was added to the mixture. The mixture was vigorously stirred and then subsequently filtered to collect the precipitated solid. This solid was further dried under vacuum to afford the title compound. MS (m/z) 340.2 [M+H]+. ¹H NMR (400 MHz, Acetonitrile-d3) δ 7.60 (d, J=1.4 Hz, 1H), 7.48 (d, J=16.7 Hz, 1H), 7.39 (s, 2H), 6.08 (d, J=16.7 Hz, 1H), 2.41 (d, J=1.4 Hz, 3H), 2.13 (s, 6H).

### Step 2, preparation of (E)-3-((4-(4-(2-cyanovinyl)-2,6-dimethylphenoxy)-5-methylfuro[2,3-d]pyrimidin-2-yl)amino)bicyclo[1.1.1]pentane-1-carbonitrile (Compound 300)

(E)-3-(4-((2-chloro-5-methylfuro[2,3-d]pyrimidin-4-yl)oxy)-3,5-dimethylphenyl)acrylonitrile (0.0974 mmol, 33 mg), 3-aminobicyclo[1.1.1]pentane-1-carbonitrile (0.292 mmol, 32 mg), cesium carbonate (0.292 mmol, 95 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.0974 mmol, 97 mg) was mixed with 1,4-dioxane (2.0 mL). The mixture was degassed by bubbling through argon for 1-2 minutes, then the mixture was sealed under argon atmosphere and stirred in a metal heating block at 60° C. for 42 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. MS (m/z) 412.3 [M+H]+. 1H NMR (400 MHz, Acetonitrile-d3) δ 7.48 (d, J=16.7 Hz, 1H), 7.39 (s, 2H), 7.22 (d, J=1.5 Hz, 1H), 6.24 (s, 1H), 6.06 (d, J=16.7 Hz, 1H), 2.33 (d, J=1.5 Hz, 3H), 2.13-2.03 (m, 12H).

### Example 301

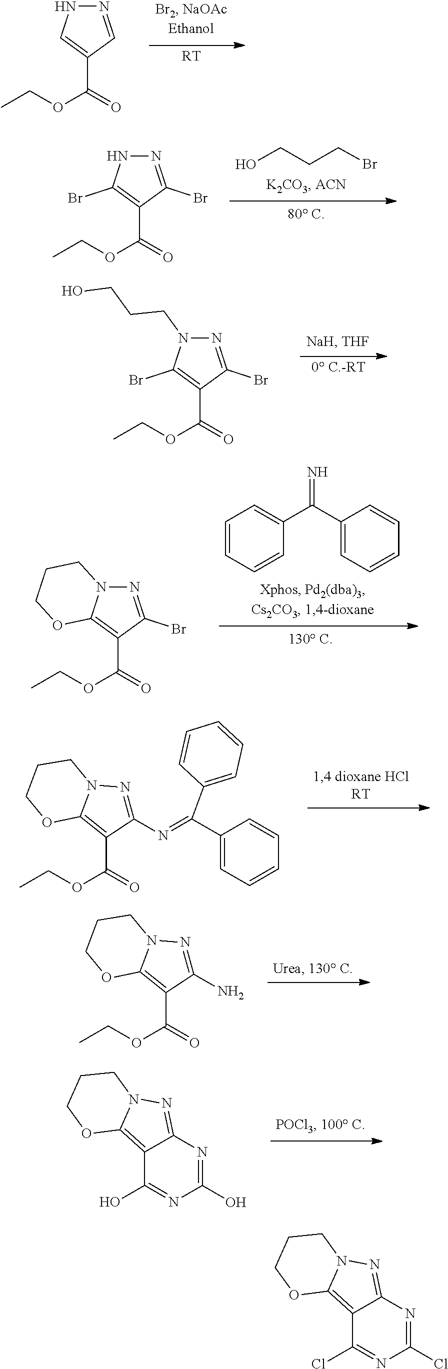

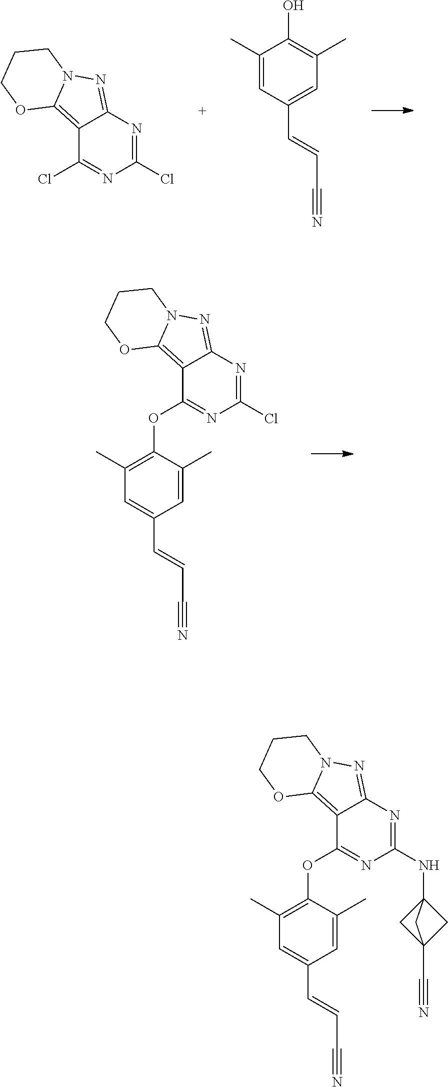

### Step 1. Preparation of ethyl 3,5-dibromo-1H-pyrazole-4-carboxylate (Intermediate 301a)

To a stirred solution of ethyl 1H-pyrazole-4-carboxylate (178 mmol, 25 g) in Ethanol (250 mL) was added sodium acetate (1178 mmol, 96.6 g) at 0-5° C. To the reaction mixture was added bromine (713 mmol, 114.2 g). After completion of the addition, the reaction mixture was allowed to warm to room temperature and stirred for 16 h at ambient temperature. The reaction mixture was poured in ice cold water and extracted with ethyl acetate. The combined organic layer was washed with ice-cold water, then brine, dried over Na₂SO₄ and evaporated under reduced pressure to obtain the title compound as a crude solid which was carried forward to next step without any additional purification. 1H NMR (400 MHz, DMSO-d6) δ 14.51 (s, 1H), 4.28-4.23 (q, 2H), 1.32-1.28 (t, 3H).

### Step 2. Preparation of ethyl 3,5-dibromo-1-(3-hydroxypropyl)-1H-pyrazole-4-carboxylate (Intermediate 301b)

To a stirred solution of ethyl 3,5-dibromo-1H-pyrazole-4-carboxylate (152 mmol, 45 g) in acetonitrile (450 mL) was added potassium carbonate (287 mmol, 40 g) and 1-bromo-3-propanol (167 mmol, 23.3 g) at ambient temperature. The mixture was stirred at 80° C. for 3h. The mixture was filtered through celite and the filtrate was distilled under reduced pressure to obtain the title compound as a crude liquid, which was carried forward to next step without any additional purification. MS (m/z) 357.1 [M+H]+. ¹H NMR (400 MHz, CDCl3) δ 4.39-4.33 (m, 4H), 3.68-3.65 (t, 2H), 2.11-2.05 (m, 2H), 1.66 (s, 1H), 1.41-1.38 (t, 3H).

### Step 3. Preparation of ethyl 2-bromo-6,7-dihydro-5H-pyrazolo[5,1-b][1,3]oxazine-3-carboxylate (Intermediate 301c)

To a stirred solution of ethyl 3,5-dibromo-1-(3-hydroxypropyl)-1H-pyrazole-4-carboxylate (164 mmol, 58 g) in tetrahydrofuran (3.4 L) at 0-5° C. was added 60% sodium hydride dispersion in mineral oil (328 mmol, 13.14 g) portion wise. After completion of the addition the mixture was allowed to stir at ambient temperature and maintained for 16 h at ambient temperature. The mixture was quenched with ice water and extracted with ethyl acetate. The combined organic layer was washed with water and brine, dried over Na₂SO₄, and evaporated under reduced pressure to obtain a crude solid. Purification by silica gel flash column chromatography (ethyl acetate in petroleum ether) yielded the title compound. MS (m/z) 275.0 [M+H]+. ¹H NMR (400 MHz, DMSO-d6) δ 4.44-4.41 (t, 2H), 4.19-4.14 (q, 2H), 4.07-4.04 (q, 2H), 2.51-2.49 (m, 2H), 1.25-1.21 (t, 3H).

### Step 4. Preparation of ethyl 2-((diphenylmethylene)amino)-6,7-dihydro-5H-pyrazolo[5,1-b][1,3]oxazine-3-carboxylate (Intermediate 301d)

1,4-dioxane was added to a three neck round bottom flask, purged with argon. This was followed by addition of ethyl 2-bromo-6,7-dihydro-5H-pyrazolo[5,1-b][1,3]oxazine-3-carboxylate (124 mmol, 34 g) and diphenylmethanimine (136 mmol, 24.7 g). The mixture was again purged with argon. This was followed by addition of Cesium carbonate (248 mmol, 80.8 g) and XPhos (12.4 mmol, 5.9 g) and Pd2(dba)3 (24.8 mmol, 22.7 g) one after another and purging of mixture with argon at ambient temperature. The mixture was heated at 130° C. for 16 h. The reaction mixture was filtered through celite and the filtrate was concentrated under reduced pressure to obtain crude material. Purification by silica gel flash column chromatography (ethyl acetate/petroleum ether) yielded the title compound. MS (m/z) 376.2 [M+H]+. ¹H NMR (400 MHz, CDCl3) δ 7.80-7.75 (m, 2H), 7.48-7.44 (m, 1H), 7.40-7.36 (m, 2H), 7.33-7.26 (m, 5H), 4.36-4.33 (t, 2H), 4.15-4.09 (q, 2H), 4.00-3.97 (t, 2H), 2.23-2.20 (m, 2H), 1.28-1.24 (t, 3H).

### Step 5. Preparation of ethyl 2-amino-6,7-dihydro-5H-pyrazolo[5,1-b][1,3]oxazine-3-carboxylate (Intermediate 301e)

To a stirred solution of ethyl 2-((diphenylmethylene)amino)-6,7-dihydro-5H-pyrazolo[5,1-b][1,3]oxazine-3-carboxylate (90.6 mmol, 34 g) in 1,4-dioxane (170 mL) was added 4M HCl in 1,4-Dioxane (408 mmol, 102 mL) at 0-5° C. After completion of the addition the mixture was allowed to stir at ambient atmosphere. The reaction mixture was stirred for 3 h at ambient temperature. The mixture was concentrated under reduced pressure. To the mixture was added water and then the mixture was extracted with Ethyl acetate. The aqueous phase was treated with sat. solution of sodium bicarbonate so that pH ˜8. The aqueous phase was extracted with Ethyl acetate. The combined organic phases were washed with brine solution, filtered over anhydrous sodium sulphate and concentrated under reduced pressure to obtain the title compound which was carried forward without further purification. MS (m/z) 212.3 [M+H]+. ¹H NMR (500 MHz, DMSO-d6) δ 5.27 (s, 2H), 4.33-4.31 (t, 2H), 4.14-4.10 (q, 2H), 3.81-3.78 (t, 2H), 2.15-2.11 (m, 2H), 1.22-1.19 (t, 3H).

### Step 6. Preparation of 7,8-dihydro-6H-pyrimido[4′,5′:3,4]pyrazolo[5,1-b][1,3]oxazine-2,4-diol (Intermediate 301f)

A mixture of ethyl 2-amino-6,7-dihydro-5H-pyrazolo[5,1-b][1,3]oxazine-3-carboxylate (23 mmol, 5.0 g) and urea (236 mmol, 14.2 g) was stirred for 24 h at 130° C. Water was added, and filtration to collect the precipitated solid yielded the title compound, which was carried forward without further purification. MS (m/z) 209.0 [M+H]+. ¹H NMR (400 MHz, DMSO-d6) δ 11.10 (s, 1H), 10.38 (s, 1H), 4.43-4.41 (t, 2H), 4.00-3.97 (t, 2H), 2.23-2.20 (m, 2H).

### Step 7. Preparation of 2,4-dichloro-7,8-dihydro-6H-pyrimido[4′,5′:3,4]pyrazolo[5,1-b][1,3]oxazine (Intermediate 301g)

A mixture of 7,8-dihydro-6H-pyrimido[4′,5′:3,4]pyrazolo[5,1-b][1,3]oxazine-2,4-diol (19.2 mmol, 3.0 g) and POCl3 (45 mL) was stirred at 100° C. for 20 h. The reaction mixture was evaporated completely under reduced pressure. To the residue was added ice cold water, then the aqueous phase was treated with sat. solution of sodium bicarbonate to reach pH ˜6 and extracted with ethyl acetate. The combined organic phase was washed with brine, filtered over anhydrous sodium sulphate and concentrated under reduced pressure to obtain crude as an off white solid. Purification by reverse-phase preparative HPLC (acetonitrile, water with 10 mM ammonium bicarbonate) yielded the title compound. MS (m/z) 245.1 [M+H]+. ¹H NMR (400 MHz, DMSO-d6) δ 4.67-4.65 (t, 2H), 4.36-4.33 (t, 2H), 2.40-2.33 (m, 2H).

### Step 8. Preparation of (E)-3-(4-((2-chloro-7,8-dihydro-6H-pyrimido[4′,5′:3,4]pyrazolo[5,1-b][1,3]oxazin-4-yl)oxy)-3,5-dimethylphenyl)acrylonitrile (Intermediate 301h)

2,4-dichloro-7,8-dihydro-6H-pyrimido[4′,5′:3,4]pyrazolo[5,1-b][1,3]oxazine (0.494 mmol, 121 mg) (E)-3-(4-hydroxy-3,5-dimethylphenyl)acrylonitrile (0.494 mmol, 86 mg) and potassium carbonate (0.494 mmol, 68 mg) were mixed with DMF (1.1 mL). The mixture was stirred in a metal heating block at 60° C. for 8 h at which point water was added to the mixture. The mixture was vigorously stirred and then subsequently filtered to collect the precipitated solid. This solid was further dried under vacuum to afford the title compound. DMF. The mixture stirred in metal heating block at 60° C. for a total of 25 hr. The mixture was diluted with water and filtered to collect the resulting precipitate, yielding the title compound which was carried forward without further purification. MS (m/z) 382.3 [M+H]+. ¹H NMR (400 MHz, Acetonitrile-d3) δ 7.47 (d, J=16.7 Hz, 1H), 7.37 (s, 2H), 6.07 (d, J=16.7 Hz, 1H), 4.63-4.54 (m, 2H), 4.33 (t, J=6.1 Hz, 2H), 2.46-2.35 (m, 2H), 2.13 (s, 6H).

### Step 9. Preparation of (E)-3-((4-(4-(2-cyanovinyl)-2,6-dimethylphenoxy)-7,8-dihydro-6H-pyrimido[4′,5′:3,4]pyrazolo[5,1-b][1,3]oxazin-2-yl)amino)bicyclo[1.1.1]pentane-1-carbonitrile (Compound 301)

(E)-3-(4-((2-chloro-7,8-dihydro-6H-pyrimido[4′,5′:3,4]pyrazolo[5,1-b][1,3]oxazin-4-yl)oxy)-3,5-dimethylphenyl)acrylonitrile (0.100 mmol, 38 mg), 3-aminobicyclo[1.1.1]pentane-1-carbonitrile (0.299 mmol, 32 mg), cesium carbonate (0.299 mmol, 97 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.0995 mmol, 99 mg) was mixed with 1,4-dioxane (2 mL). The mixture was degassed by bubbling through argon for 1-2 minutes, then the mixture was sealed under argon atmosphere and stirred in a metal heating block at 60° C. for 24 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. MS (m/z) 454.2 [M+H]+. ¹H NMR (400 MHz, DMSO-d6) δ 7.72 (s, 1H), 7.61 (d, J=16.6 Hz, 1H), 7.51 (s, 2H), 6.42 (d, J=16.7 Hz, 1H), 4.55 (t, J=5.1 Hz, 2H), 4.17 (t, J=6.0 Hz, 2H), 2.33-2.27 (m, 2H), 2.09-2.04 (m, 12H).

### Example 302

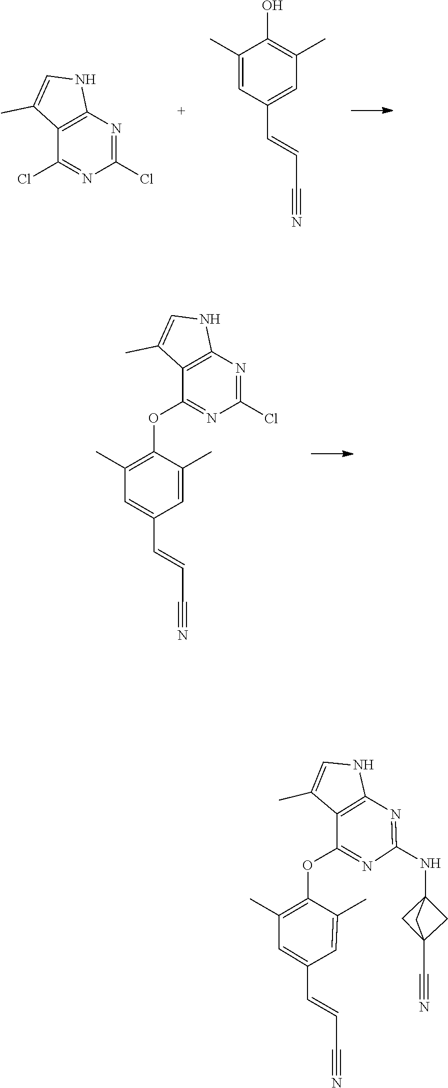

### Step 1, preparation of (E)-3-(4-((2-chloro-5-methyl-7H-pyrrolo[2,3-d]pyrimidin-4-yl)oxy)-3,5-dimethylphenyl)acrylonitrile (Intermediate 302a)

(E)-3-(4-hydroxy-3,5-dimethylphenyl)acrylonitrile (0.433 mmol, 75 mg), 2,4-dichloro-5-methyl-7H-pyrrolo[2,3-d]pyrimidine (0.433 mmol, 88 mg), and potassium carbonate (0.542 mmol, 75 mg) were mixed in DMF (1.1 mL). The mixture was stirred in a metal heating block at 60° C. for 23 h. The mixture was partitioned between ethyl acetate and water, then the organic phase with washed more water, then brine. The organic phase dried over MgSO4, filtered, and concentrated in vacuo. Silica gel flash column chromatography (EtOAc in hexane) yielded the title compound. MS (m/z) 339.2 [M+H]+. ¹H NMR (400 MHz, acetonitrile-d3) δ 9.79 (s, 1H), 7.47 (d, J=16.7 Hz, 1H), 7.37 (s, 2H), 7.06 (s, 1H), 6.06 (d, J=16.7 Hz, 1H), 2.47 (s, 3H), 2.14 (s, 6H).

### Step 2, preparation of (E)-3-((4-(4-(2-cyanovinyl)-2,6-dimethylphenoxy)-5-methyl-7H-pyrrolo[2,3-d]pyrimidin-2-yl)amino)bicyclo[1.1.1]pentane-1-carbonitrile (Compound 302)

(E)-3-(4-((2-chloro-5-methyl-7H-pyrrolo[2,3-d]pyrimidin-4-yl)oxy)-3,5-dimethylphenyl)acrylonitrile (0.0974 mmol, 33 mg), 3-aminobicyclo[1.1.1]pentane-1-carbonitrile (0.292 mmol, 32 mg), cesium carbonate (0.292 mmol, 95 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.0974 mmol, 97 mg) was mixed with 1,4-dioxane (2 mL). The mixture was degassed by bubbling through argon for 1-2 minutes, then the mixture was sealed under argon atmosphere and stirred in a metal heating block at 60° C. for 96 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. ¹H NMR (400 MHz, acetonitrile-d3) δ 8.97 (s, 1H), 7.48 (d, J=16.7 Hz, 1H), 7.38 (s, 2H), 6.72-6.61 (m, 1H), 6.05 (d, J=16.7 Hz, 1H), 5.82 (s, 1H), 2.39 (s, 3H), 2.12 (s, 6H), 2.09 (s, 6H). MS (m/z) 411.3 [M+H]+.

### Example 303

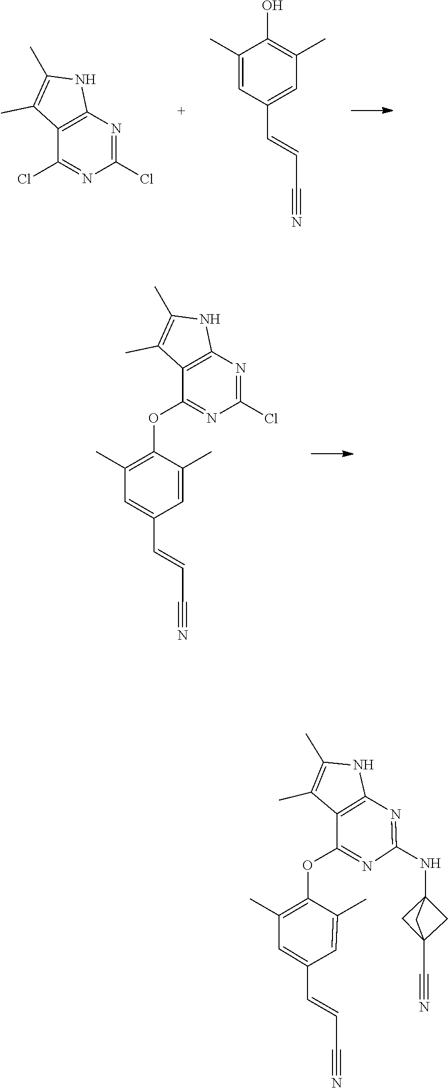

### Step 1, preparation of (E)-3-(4-((2-chloro-5,6-dimethyl-7H-pyrrolo[2,3-d]pyrimidin-4-yl)oxy)-3,5-dimethylphenyl)acrylonitrile (Intermediate 303a)

(E)-3-(4-hydroxy-3,5-dimethylphenyl)acrylonitrile (0.433 mmol, 75 mg), 2,4-dichloro-5,6-dimethyl-7H-pyrrolo[2,3-d]pyrimidine (0.433 mmol, 94 mg), and potassium carbonate (0.542 mmol, 75 mg) were mixed in DMF (1.1 mL). The mixture was stirred in a metal heating block at 60° C. for 18 days, with periodic addition of more potassium carbonate. The mixture was vigorously stirred and then subsequently filtered to collect the precipitated solid. This solid was further dried under vacuum to afford the title compound. MS (m/z) 353.2 [M+H]+. ¹H NMR (400 MHz, DMSO-d6) δ 12.04 (s, 1H), 7.61 (d, J=16.7 Hz, 1H), 7.49 (s, 2H), 6.43 (d, J=16.7 Hz, 1H), 2.34 (s, 3H), 2.32 (s, 3H), 2.08 (s, 6H).

### Step 2, preparation of (E)-3-((4-(4-(2-cyanovinyl)-2,6-dimethylphenoxy)-5,6-dimethyl-7H-pyrrolo[2,3-d]pyrimidin-2-yl)amino)bicyclo[1.1.1]pentane-1-carbonitrile (Compound 303)

(E)-3-(4-((2-chloro-5,6-dimethyl-7H-pyrrolo[2,3-d]pyrimidin-4-yl)oxy)-3,5-dimethylphenyl)acrylonitrile (0.0709 mmol, 25 mg), 3-aminobicyclo[1.1.1]pentane-1-carbonitrile (0.213 mmol, 23 mg), cesium carbonate (0.213 mmol, 69 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.0709 mmol, 71 mg) was mixed with 1,4-dioxane (1.4 mL). The mixture was degassed by bubbling through argon for 1-2 minutes, then the mixture was sealed under argon atmosphere and stirred in a metal heating block at 60° C. for 42 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. MS (m/z) 425.3 [M+H]+. ¹H NMR (400 MHz, acetonitrile-d3) δ 8.93 (s, 1H), 7.48 (d, J=16.7 Hz, 1H), 7.37 (s, 2H), 6.04 (d, J=16.7 Hz, 1H), 5.74 (s, 1H), 2.31 (s, 3H), 2.23 (s, 3H), 2.11 (s, 6H), 2.07 (s, 6H).

### Example 304

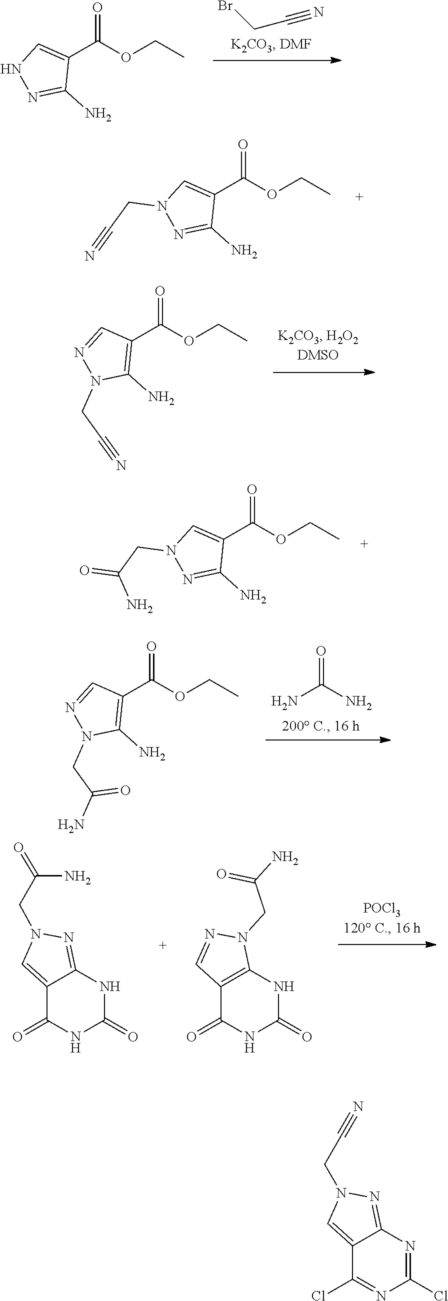

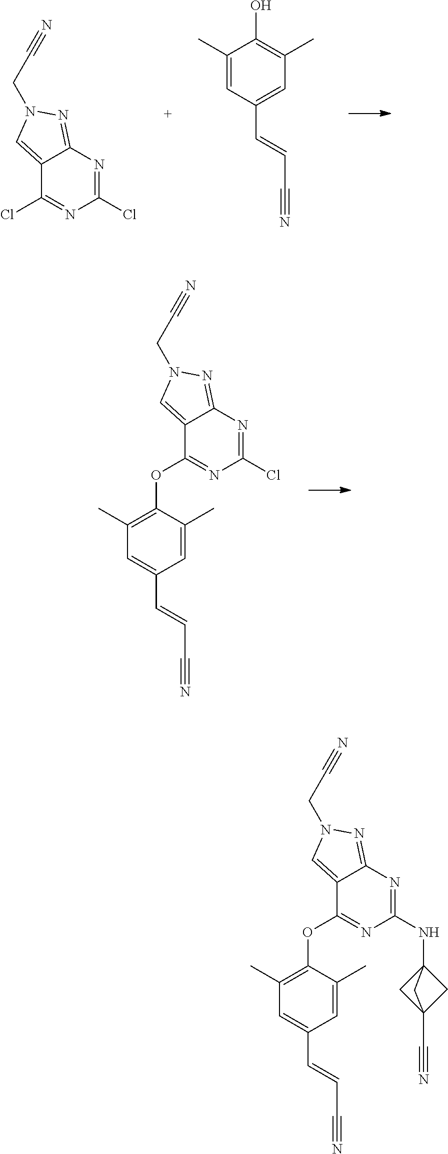

### Step 1. Preparation of ethyl 3-amino-1-(cyanomethyl)-1H-pyrazole-4-carboxylate (Intermediate 304a)

To a solution of ethyl 3-amino-1H-pyrazole-4-carboxylate (128 mmol, 20 g) in DMF (200 mL) was added potassium carbonate (255 mmol, 35.3 g), and 2-bromoacetonitrile (153 mmol, 18.2 g) dropwise under nitrogen atmosphere at 0° C. The reaction mixture was stirred for 16 h at ambient temperature. The mixture was quenched with ice water and stirred for 5 min, then extracted with EtOAc. The combined organic layer was washed with brine solution, dried over Na₂SO₄, and evaporated under reduced pressure. Purification by silica gel flash column chromatography (ethyl acetate in petroleum ether) afforded the title compound as a mixture with the isomeric ethyl 5-amino-1-(cyanomethyl)-1H-pyrazole-4-carboxylate. MS (m/z) 195.1 [M+H]+.

### Step 2. Preparation of ethyl 3-amino-1-(2-amino-2-oxoethyl)-1H-pyrazole-4-carboxylate (Intermediate 304b)

To stirred solution of a mixture of ethyl 3-amino-1H-pyrazole-4-carboxylate and isomeric ethyl 5-amino-1-(cyanomethyl)-1H-pyrazole-4-carboxylate (100 mmol, 20 g) in DMSO (200 mL) was added potassium carbonate (300 mmol, 42 g) portion wise at ambient temperature, then 30% H₂O₂ (50 mmol, 5.1 g) was added slowly dropwise. The reaction mixture was stirred for 16 h at ambient temperature. The mixture was diluted with water and extracted with 5% MeOH/DCM. The organic phase was dried over Na₂SO₄, and evaporated under reduced pressure. Purification by silica gel flash column chromatography (MeOH/DCM) afforded the title compound as a mixture with the isomeric ethyl 5-amino-1-(2-amino-2-oxoethyl)-1H-pyrazole-4-carboxylate. MS (m/z) 213.1 [M+H]+.

### Step 3. Preparation of 2-(4,6-dioxo-4,5,6,7-tetrahydro-2H-pyrazolo[3,4-d]pyrimidin-2-yl)acetamide (Intermediate 304c)

A mixture of ethyl 3-amino-1-(2-amino-2-oxoethyl)-1H-pyrazole-4-carboxylate and the isomeric ethyl 5-amino-1-(2-amino-2-oxoethyl)-1H-pyrazole-4-carboxylate (571 mmol, 12 g) and urea (5710 mmol, 34.2 g) was heated to 200° C. for 16 h. After cooling back to ambient temperature, water (24 mL) was added and heated to 200° C. for 1 h. The mixture was cooled to ambient temperature, the resulting solid collected by filtration to afford the title compound as a mixture with the isomeric 2-(4,6-dioxo-4,5,6,7-tetrahydro-1H-pyrazolo[3,4-d]pyrimidin-1-yl)acetamide. MS (m/z) 210.1 [M+H]+.

### Step 4. Preparation of 2-(4,6-dichloro-2H-pyrazolo[3,4-d]pyrimidin-2-yl)acetonitrile (Intermediate 304d)

A mixture of 2-(4,6-dioxo-4,5,6,7-tetrahydro-2H-pyrazolo[3,4-d]pyrimidin-2-yl)acetamide and isomeric 2-(4,6-dioxo-4,5,6,7-tetrahydro-1H-pyrazolo[3,4-d]pyrimidin-1-yl)acetamide (23.9 mmol, 5.0 g) and POCl3 (100 mL) was stirred at 120° C. for 16 hr. The mixture was concentrated in vacuo and treated with cold saturated NaHCO₃ solution, then extracted with EtOAc. The combined organic extracts were dried over anhydrous Na₂SO₄ and evaporated. Purification by silica gel flash column chromatography (ethyl acetate/petroleum ether) afforded the title compound. MS (m/z) 228.0 [M+H]+. ¹H NMR (400 MHz, DMSO-d6) δ 9.12 (s, 1H), 5.93 (s, 2H).

### Step 5. Preparation of (E)-3-(4-((6-chloro-2-(cyanomethyl)-2H-pyrazolo[3,4-d]pyrimidin-4-yl)oxy)-3,5-dimethylphenyl)acrylonitrile (Intermediate 304e)

(E)-3-(4-hydroxy-3,5-dimethylphenyl)acrylonitrile (0.433 mmol, 75 mg), 2-(4,6-dichloro-2H-pyrazolo[3,4-d]pyrimidin-2-yl)acetonitrile (0.434 mmol, 99 mg), and potassium carbonate (0.434 mmol, 60 mg) were mixed in DMF (1.1 mL). The mixture was stirred at 20° C. for 2 h. The mixture was partitioned between ethyl acetate and water, then the organic phase with washed more water, then brine. The organic phase dried over MgSO4, filtered, and concentrated in vacuo. Silica gel flash column chromatography (EtOAc in hexane) yielded the title compound. MS (m/z) 365.1 [M+H]+. ¹H NMR (400 MHz, Acetonitrile-d3) δ 8.41 (s, 1H), 7.48 (d, J=16.7 Hz, 1H), 7.40 (s, 2H), 6.09 (d, J=16.7 Hz, 1H), 5.48 (s, 2H), 2.13 (s, 6H).

### Step 6. Preparation of (E)-3-((2-(cyanomethyl)-4-(4-(2-cyanovinyl)-2,6-dimethylphenoxy)-2H-pyrazolo[3,4-d]pyrimidin-6-yl)amino)bicyclo[1.1.1]pentane-1-carbonitrile (Compound 304)

(E)-3-(4-((6-chloro-2-(cyanomethyl)-2H-pyrazolo[3,4-d]pyrimidin-4-yl)oxy)-3,5-dimethylphenyl)acrylonitrile (0.0192 mmol, 7 mg), 3-aminobicyclo[1.1.1]pentane-1-carbonitrile (0.0959 mmol, 10.4 mg), cesium carbonate (0.0576 mmol, 19 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.0384 mmol, 38 mg) was mixed with 1,4-dioxane (0.30 mL). The mixture was degassed by bubbling through argon for 1-2 minutes, then the mixture was sealed under argon atmosphere and stirred in a metal heating block at 60° C. for 9 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. MS (m/z) 437.2 [M+H]+. ¹H NMR (400 MHz, acetonitrile-d3) δ 8.15 (s, 1H), 7.48 (d, J=16.7 Hz, 1H), 7.38 (s, 2H), 6.22 (s, 1H), 6.07 (d, J=16.7 Hz, 1H), 5.31 (s, 2H), 2.13-2.06 (m, 12H).

### Example 305

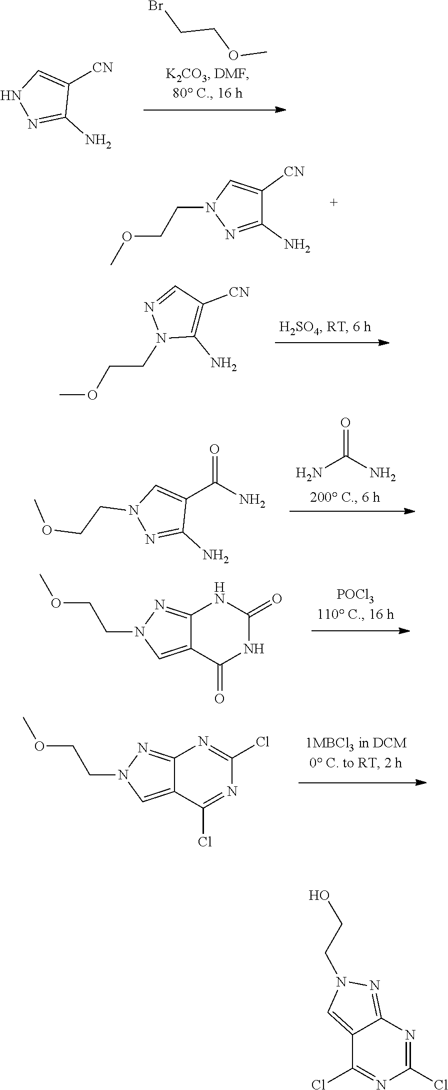

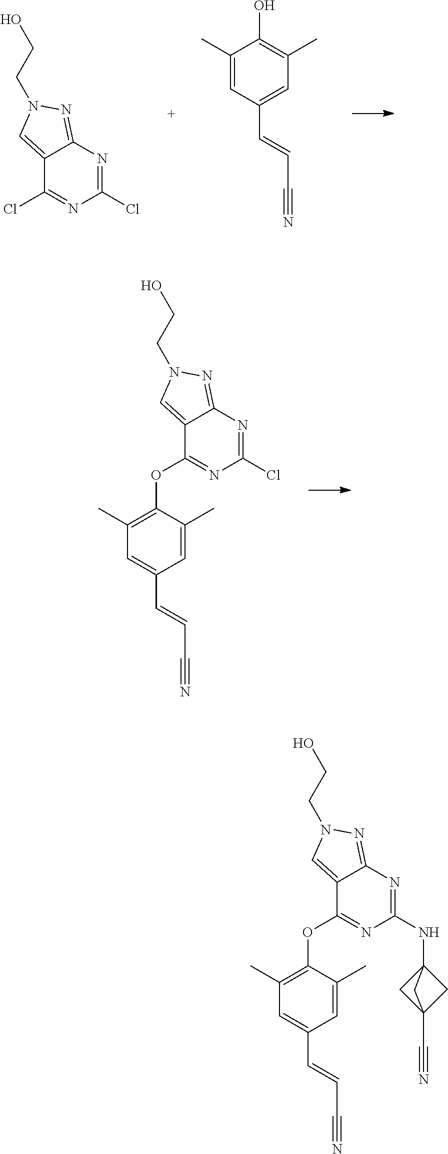

### Step 1. Preparation of 3-amino-1-(2-methoxyethyl)-1H-pyrazole-4-carbonitrile (Intermediate 305a)

To a mixture of 3-amino-1H-pyrazole-4-carbonitrile (90 mmol, 10 g) in DMF (100 mL) was added Potassium carbonate (130 mmol, 19.16 g), then 1-bromo-2-methoxyethane (120 mmol, 16.6 g) dropwise under nitrogen atmosphere at 0° C. The reaction mixture was stirred at 80° C. for 16 h. The mixture was treated with ice water and stirred for 5 min. then extracted with EtOAc. The combined organic phases were washed with brine solution, dried over Na₂SO₄, and concentrated under reduced pressure. Silica gel flash column chromatography (EtOAc/petroleum ether) afforded the title compound. 1H NMR (400 MHz, CDCl3) δ 7.59 (s, 1H), 4.17-4.06 (m, 4H), 3.72-3.66 (m, 2H), 3.34 (s, 3H). MS (m/z) 167.1 [M+H]+.

### Step 2. Preparation of 3-amino-1-(2-methoxyethyl)-1H-pyrazole-4-carboxamide (Intermediate 305b)

A mixture of 3-amino-1-(2-methoxyethyl)-1H-pyrazole-4-carbonitrile (20 mmol, 3.4 g) and concentrated sulfuric acid (4 mL) was stirred at ambient temperature under nitrogen atmosphere for 6 hr. The mixture was added to concentrated ammonium hydroxide solution very slowly at 0° C. and the aqueous phase was extracted with EtOAc. The combined organic extracts were washed with brine (50 mL), and dried over anhydrous Na₂SO₄ and concentrated in vacuo to afford the title compound. MS (m/z) 185.1 [M+H]+. ¹H NMR (400 MHz, DMSO-d6) δ 7.87 (s, 1H), 7.62 (s, 1H), 7.22 (s, 1H), 5.35 (s, 2H), 3.99-3.97 (t, 2H), 3.61-3.58 (t, 2H), 3.22 (s, 3H).

### Step 3. Preparation of 2-(2-methoxyethyl)-2,7-dihydro-4H-pyrazolo[3,4-d]pyrimidine-4,6(5H)-dione (Intermediate 305c)

A mixture of 3-amino-1-(2-methoxyethyl)-1H-pyrazole-4-carboxamide (13 mmol, 2.5 g) and urea (130 mmol, 8.15 g) was heated to 200° C. for 6 h and then cooled to ambient temperature. Water (7 mL) was added and heated to 200° C. for 1 h. The mixture was allowed to cool to ambient temperature, and the resulting solid was collected via filtration and dried under vacuum to afford the title compound. MS (m/z) 211.1 [M+H]+. ¹H NMR (400 MHz, DMSO-d6) δ 11.35 (s, 1H), 10.68 (s, 1H), 8.29 (s, 1H), 4.26-4.24 (t, 2H), 3.70-3.67 (t, 2H), 3.22 (s, 3H).

### Step 4. Preparation of 4,6-dichloro-2-(2-methoxyethyl)-2H-pyrazolo[3,4-d]pyrimidine (Intermediate 305d)

A mixture of 2-(2-methoxyethyl)-2,7-dihydro-4H-pyrazolo[3,4-d]pyrimidine-4,6(5H)-dione (7.6 mmol, 1.6 g) and phosphoryl chloride (80 mL) was heated to 120° C. for 16 h. The mixture was concentrated to remove POCl3, then cooled to 0° C. and treated with cold saturated aqueous Na₂CO3. The mixture was extracted with EtOAc. The combined organic extracts were dried over anhydrous Na₂SO₄ and concentrated in vacuo. Silica gel flash column chromatography (ethyl acetate/petroleum ether) afforded the title compound. MS (m/z) 247.2 [M+H]+. ¹H NMR (400 MHz, DMSO-d6) δ 9.04 (s, 1H), 4.67-4.66 (t, 2H), 3.88-3.85 (t, 2H), 3.24 (s, 3H).

### Step 5. Preparation of 2-(4,6-dichloro-2H-pyrazolo[3,4-d]pyrimidin-2-yl)ethan-1-ol (Intermediate 305e)

To a stirred mixture of 4,6-dichloro-2-(2-methoxyethyl)-2H-pyrazolo[3,4-d]pyrimidine (4.0 mmol, 1.2 g) in DCM (12 mL) was added Boron trichloride, 1M in dichloromethane (8.0 mmol, 9.75 mL), at 0° C. slowly and the mixture was stirred at 0° C. for 2 h. The mixture was concentrated in vacuo, then cooled to 0° C. and treated with cold saturated aqueous NaHCO₃. The aqueous phase was extracted with EtOAc. The combined organic extracts were dried over anhydrous Na₂SO₄ and concentrated in vacuo. Silica gel flash column chromatography (ethyl acetate/petroleum ether) afforded the title compound. MS (m/z) 233.0 [M+H]+. ¹H NMR (400 MHz, DMSO-d6) δ 9.01 (s, 1H), 4.54-4.52 (t, 2H), 3.91-3.88 (t, 2H).

### Step 6. Preparation of (E)-3-(4-((6-chloro-2-(2-hydroxyethyl)-2H-pyrazolo[3,4-d]pyrimidin-4-yl)oxy)-3,5-dimethylphenyl)acrylonitrile (Intermediate 305f)

(E)-3-(4-hydroxy-3,5-dimethylphenyl)acrylonitrile (0.433 mmol, 75 mg), 2-(4,6-dichloro-2H-pyrazolo[3,4-d]pyrimidin-2-yl)ethan-1-ol (0.433 mmol, 101 mg), and potassium carbonate (0.542 mmol, 75 mg) were mixed in DMF (1.1 mL). The mixture was stirred at 20° C. for 18 hr. The mixture was vigorously stirred and then subsequently filtered to collect the precipitated solid. This solid was further dried under vacuum to afford the title compound. MS (m/z) 370.1 [M+H]+. ¹H NMR (400 MHz, DMSO-d6) δ 8.81 (s, 1H), 7.63 (d, J=16.7 Hz, 1H), 7.53 (s, 2H), 6.46 (d, J=16.7 Hz, 1H), 5.04 (s, 1H), 4.49 (t, J=5.2 Hz, 2H), 3.90 (s, 2H), 2.09 (s, 6H).

### Step 7. Preparation of (E)-3-((4-(4-(2-cyanovinyl)-2,6-dimethylphenoxy)-2-(2-hydroxyethyl)-2H-pyrazolo[3,4-d]pyrimidin-6-yl)amino)bicyclo[1.1.1]pentane-1-carbonitrile (Compound 305)

(E)-3-(4-((6-chloro-2-(2-hydroxyethyl)-2H-pyrazolo[3,4-d]pyrimidin-4-yl)oxy)-3,5-dimethylphenyl)acrylonitrile (0.135 mmol, 50 mg), 3-aminobicyclo[1.1.1]pentane-1-carbonitrile (0.406 mmol, 44 mg), cesium carbonate (0.406 mmol, 132 mg), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (rac-BINAP Pd third generation precatalyst, 0.135 mmol, 134 mg) was mixed with 1,4-dioxane (2.6 mL). The mixture was degassed by bubbling through argon for 1-2 minutes, then the mixture was sealed under argon atmosphere and stirred in a metal heating block at 60° C. for 25 hr. The mixture was then diluted with EtOAc and filtered to remove insoluble solids, then concentrated in vacuo. Silica gel flash column chromatography (EtOAc in hexane) yielded the crude title compound, which was then further purified by preparative reverse-phase HPLC (acetonitrile in water, 0.1% trifluoracetic acid) to yield the title compound. MS (m/z) 442.3 [M+H]+. ¹H NMR (400 MHz, CD3CN) δ 8.07 (s, 1H), 7.48 (d, J=16.7 Hz, 1H), 7.38 (s, 2H), 6.15-6.00 (m, 2H), 4.33 (t, J=5.1 Hz, 2H), 3.94 (t, J=5.1 Hz, 2H), 3.12 (s, 3H), 2.39-2.06 (m, 12H).

### Example 306

### Preparation of 4-hydroxy-3-methyl-1-naphthaldehyde (Intermediate 306a)

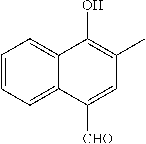

To a solution of 2-methylnaphthalen-1-ol (5.7 g, 49.6 mmol, 1.6 eq) in DCM (320 mL) at −20° C. was added TiCl₄ (1 M in DCM, 95 mL, 94.8 mmol, 3.0 eq) dropwise. After the addition, dichloro(methoxy)methane (5.0 g, 31.6 mmol, 1.0 eq) was added in turn. The reaction mixture was stirred at −20° C. for 30 min, then warmed up to room temperature slowly with stirring at rt for another 30 min. the reaction mixture was re-cooled to 0° C., ice cold water (100 mL) was added. After 10 min of vigorous stirring. 1 N aq. HCl (200 mL) was added. The mixture was stirred until all salts were dissolved. The reaction mixture was extracted with DCM (100 mL×2), washed with brine (50 mL×1), dried over Na₂SO₄, then filtrated and concentrated under reduced pressure to dryness. The residue was purified by column chromatography (Petroleum: EtOAc=4:1) to give 4-hydroxy-3-methyl-1-naphthaldehyde (3.5 g, 59.5%) as a brown solid. MS (m/z) 187.0 ([M+H]⁺). ¹H NMR (400 MHz, DMSO-d₆): δ ppm 10.12 (s, 1H), 9.17 (d, J=8.4 Hz, 1H), 8.33 (d, J=8.2 Hz, 1H), 7.96 (s, 1H), 7.68-7.55 (m, 2H), 2.43 (s, 3H).

### Preparation of (E)-3-(4-hydroxy-3-methylnaphthalen-1-yl)acrylonitrile (Intermediate 306b)

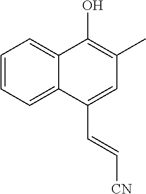 [SMILES: Cc1cc(/C=C/C#N)c2ccccc2c1O]

To a suspension of NaH (1.9 g, 48.3 mmol, 2.5 eq) in an oven-dried 250 mL three-neck round bottom flask was added THF (76 mL). The mixture was cooled to 0° C., diethyl (cyanomethyl)phosphonate (6.8 g, 38.7 mmol, 2.0 eq) was added dropwise. The reaction mixture was stirred at 0° C. for 30 min, 4-hydroxy-3-methyl-1-naphthaldehyde (3.6 g, 19.3 mmol, 1.0 eq) was added by dropwise. After the addition. The solution was warmed to rt until no starting materials by TLC. The mixture was quenched by water (20 mL), extracted by EtOAc (50 mL×2), washed by brine (50 mL×1), dried over Na₂SO₄, then filtrated and concentrated under reduced pressure to dryness. The residue was triturated with (Petroleum: EtOAc=10:1, 100 mL) to give (E)-3-(4-hydroxy-3-methyl-1-naphthyl)prop-2-enenitrile (1.0 g, 24.7%) as a yellow solid. MS (m/z) 208.1 ([M−H]⁻). ¹H NMR (400 MHz, DMSO-d₆): δ ppm 9.81 (s, 1H), 8.39 (d, J=16.3 Hz, 1H), 8.25 (dd, J=6.1, 2.9 Hz, 2H), 7.85 (s, 1H), 7.58-7.48 (m, 2H), 6.34 (d, J=16.3 Hz, 1H), 2.38 (s, 3H).

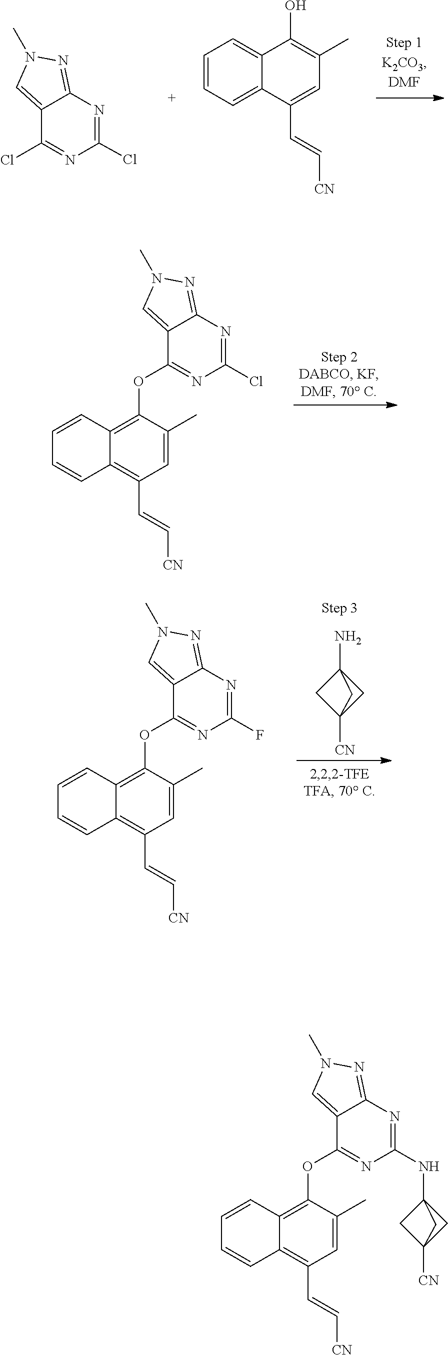

Step 1: preparation of (E)-3-[4-(6-chloro-2-methyl-pyrazolo[3,4-d]pyrimidin-4-yl)oxy-3-methyl-1-naphthyl]prop-2-enenitrile (Intermediate 306c): 4,6-dichloro-2-methyl-pyrazolo[3,4-d]pyrimidine (0.266 mmol, 100 mg), potassium carbonate (0.739 mmol, 102 mg), and (E)-3-(4-hydroxy-3-methyl-1-naphthyl)prop-2-enenitrile were added to a 20 mL scintillation vial charged with a stir bar and subsequently suspended in DMF (5.0 mL). After 16 h, LCMS revealed full conversion to the title product. Water (20 mL) was added to precipitate out the product, which was filtered, collected, and dried to provide (E)-3-[4-(6-chloro-2-methyl-pyrazolo[3,4-d]pyrimidin-4-yl)oxy-3-methyl-1-naphthyl]prop-2-enenitrile. MS (m/z) 375.9/377.9 [M+H]+

Step 2: preparation of (E)-3-[4-(6-fluoro-2-methyl-pyrazolo[3,4-d]pyrimidin-4-yl)oxy-3-methyl-1-naphthyl]prop-2-enenitrile (Intermediate 306d): (E)-3-[4-(6-chloro-2-methyl-pyrazolo[3,4-d]pyrimidin-4-yl)oxy-3-methyl-1-naphthyl]prop-2-enenitrile (0.266 mmol, 100 mg), potassium fluoride (2.13 mmol, 124 mg), and DABCO (0.133 mmol, 14.9 mg) were added to a 20 mL scintillation vial charged with a stir bar. The resulting mixture was suspended in DMF (1.7 mL) and heated to 70° C. for 4 h. After cooling to room temperature, water (20 mL) was added to precipitate out the product. The precipitate was then filtered, dried, and collected to provide the intermediate aryl fluoride. MS (m/z) 360.0 [M+H]+.

Step 3: preparation of 3-[[4-[[4-[(E)-2-cyanovinyl]-2-methyl-1-naphthyl]oxy]-2-methyl-pyrazolo[3,4-d]pyrimidin-6-yl]amino]bicyclo[1.1.1]pentane-1-carbonitrile: (E)-3-[4-(6-fluoro-2-methyl-pyrazolo[3,4-d]pyrimidin-4-yl)oxy-3-methyl-1-naphthyl]prop-2-enenitrile (45.0 mg, 0.125 mmol), 3-aminobicyclo[1.1.1]pentane-1-carbonitrile (135 mg, 1.25 mmol), and TFA (0.143 g, 1.25 mmol) were dissolved in 2,2,2-trifluorethanol (1 mL) inside of a microwave tube charged with a stir bar. This was then heated to 70° C. overnight after sealing the tube shut, at which point full conversion was achieved to the product. The solvents were removed in vacuo and saturated aq. NaHCO₃ (5 mL) was added along with DCM (5 mL). The layers were separated, and the organics were collected, dried with Na₂SO₄, and filtered to provide a crude. The resulting residue was then purified by flash column chromatography (0% to 100% Ethyl acetate in DCM) to provide the title compound. MS (m/z) 448.0 [M+H]+. ¹H NMR (400 MHz, DMSO) δ 8.62 (s, 1H), 8.55 (d, J=16.4 Hz, 1H), 8.40 (d, J=8.5 Hz, 1H), 8.04 (s, 1H), 7.77-7.70 (m, 1H), 7.62 (t, J=7.5 Hz, 1H), 7.56 (t, J=7.5 Hz, 1H), 6.56 (d, J=16.3 Hz, 1H), 4.06 (s, 3H), 2.28 (s, 3H), 2.25-0.91 (brs, 6H).

### Example 308

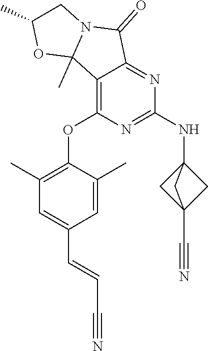 [SMILES: Cc1cc(/C=C/C#N)cc(C)c1Oc1nc(NC23CC(C#N)(C2)C3)nc2c1C1(C)O[C@H](C)CN1C2=O]

3-(((6R)-4-(4-((E)-2-cyanovinyl)-2,6-dimethylphenoxy)-4b,6-dimethyl-9-oxo-4b,6,7,9-tetrahydrooxazolo[3′,2′:1,5]pyrrolo[3,4-d]pyrimidin-2-yl)amino)bicyclo[1.1.1]pentane-1-carbonitrile (Compound 308) was made using analogous methods to that of Compound 248, Compound 249, and Compound 251.

### Example 312

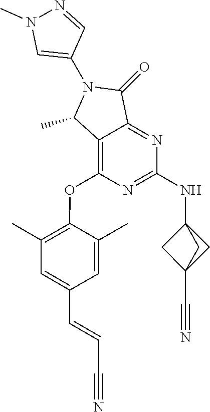 [SMILES: Cc1cc(/C=C/C#N)cc(C)c1Oc1nc(NC23CC(C#N)(C2)C3)nc2c1[C@H](C)N(c1cnn(C)c1)C2=O]

(S,E)-3-((4-(4-(2-cyanovinyl)-2,6-dimethylphenoxy)-5-methyl-6-(1-methyl-1H-pyrazol-4-yl)-7-oxo-6,7-dihydro-5H-pyrrolo[3,4-d]pyrimidin-2-yl)amino)bicyclo[1.1.1]pentane-1-carbonitrile (Compound 312) was made using analogous methods to that of Compound 230.

### Example 313

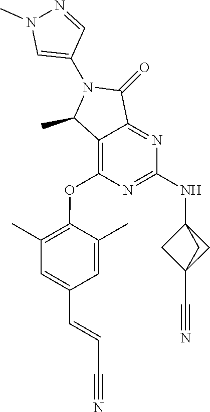 [SMILES: Cc1cc(/C=C/C#N)cc(C)c1Oc1nc(NC23CC(C#N)(C2)C3)nc2c1[C@@H](C)N(c1cnn(C)c1)C2=O]

(R,E)-3-((4-(4-(2-cyanovinyl)-2,6-dimethylphenoxy)-5-methyl-6-(1-methyl-1H-pyrazol-4-yl)-7-oxo-6,7-dihydro-5H-pyrrolo[3,4-d]pyrimidin-2-yl)amino)bicyclo[1.1.1]pentane-1-carbonitrile (Compound 313) was made using analogous methods to that of Compound 230.

### Example 314

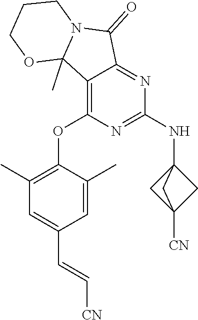 [SMILES: Cc1cc(/C=C/C#N)cc(C)c1Oc1nc(NC23CC(C#N)(C2)C3)nc2c1C1(C)OCCCN1C2=O]

### Preparation of (E)-3-((4-(4-(2-cyanovinyl)-2,6-dimethylphenoxy)-4b-methyl-10-oxo-4b,7,8,10-tetrahydro-6H-pyrimido[5′,4′:3,4]pyrrolo[2,1-b][1,3]oxazin-2-yl)amino)bicyclo[1.1.1]pentane-1-carbonitrile (Compound 314)

Compound 274 was prepared using the procedure described for the synthesis of Compound 235 with the modification that 3-aminopropan-1-ol was used in place of (R)-3-amino-2-methoxypropan-1-ol. MS (m/z): 482.92 [M+H]⁺. 1H NMR (400 MHz, DMSO-d6) δ 8.74 (s, 1H), 7.72-7.43 (m, 3H), 6.44 (d, J=16.7 Hz, 1H), 4.24-4.06 (m, 2H), 3.87 (dd, J=12.4, 4.2 Hz, 1H), 3.40 (ddd, J=15.5, 12.2, 3.8 Hz, 1H), 2.11 (s, 3H), 2.05 (s, 3H), 1.96 (s, 6H), 1.91 (s, 3H), 1.68-1.50 (m, 2H).

### Examples 315 and 316

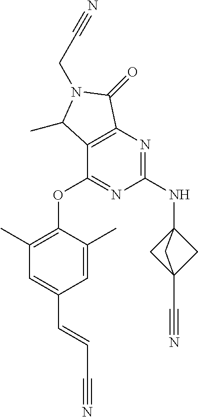 [SMILES: Cc1cc(/C=C/C#N)cc(C)c1Oc1nc(NC23CC(C#N)(C2)C3)nc2c1C(C)N(CC#N)C2=O]

(E)-3-((6-(cyanomethyl)-4-(4-(2-cyanovinyl)-2,6-dimethylphenoxy)-5-methyl-7-oxo-6,7-dihydro-5H-pyrrolo[3,4-d]pyrimidin-2-yl)amino)bicyclo[1.1.1]pentane-1-carbonitrile (Compound 315) was synthesized using the procedure described for Compound 137 with the modification that aminoacetonitrile was used in lieu of ammonium acetate and EtOH was used in place of MeOH and Et₂O and hexanes were added to the mixture once cooled to room temperature prior to filtration. Compound 316 and Compound 317 were isolated from the mixture via preparative HPLC (0-100% MeCN/H₂O, 0.1% TFA) as their TFA salts. MS (m/z) 466.31 [M+H]+. ¹H NMR (400 MHz, DMSO-d6) δ 9.08 (br s, 1H) 7.62 (d, J=16.9 Hz, 1H), 7.54 (s, 2H), 6.42 (d, J=16.7 Hz, 1H), 5.00 (s, 1H), 4.30 (d, J=5.7 Hz, 2H), 2.07 (d, J=8.1 Hz, 6H), 1.94 (s, 6H), 1.53 (d, J=6.5 Hz, 3H).

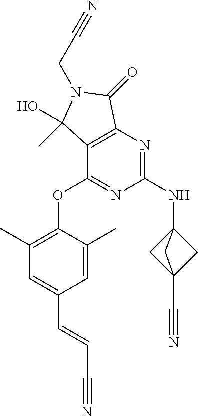 [SMILES: Cc1cc(/C=C/C#N)cc(C)c1Oc1nc(NC23CC(C#N)(C2)C3)nc2c1C(C)(O)N(CC#N)C2=O]

(E)-3-((6-(cyanomethyl)-4-(4-(2-cyanovinyl)-2,6-dimethylphenoxy)-5-hydroxy-5-methyl-7-oxo-6,7-dihydro-5H-pyrrolo[3,4-d]pyrimidin-2-yl)amino)bicyclo[1.1.1]pentane-1-carbonitrile (Compound 316): MS (m/z) 482.01 [M+H]+. ¹H NMR (400 MHz, DMSO-d6) δ 8.75 (s, 1H), 7.64 (d, J=16.9 Hz, 1H), 7.57 (s, 2H), 6.76 (s, 1H), 6.44 (d, J=16.7 Hz, 1H), 4.57 (s, 2H), 2.09 (d, J=7.6 Hz, 6H), 1.97 (s, 6H), 1.81 (s, 3H).

### Additional Examples

The following additional examples may be made using analogous methods. For instance, and further to the disclosure above, the synthesis of isoindolinone derivates has been reported in the literature, see, e.g., Sagirova, Z. R., Starodubtseva, E. V., Turova, O. V. et al. Russ Chem Bull (2013), 62, 1032-1037. Sprengeler, Paul; Reich, Siegried; et al. WO2017/075394A1 (2017).

### Compounds 156a and 156b: (R,E)-3-((4-(2-chloro-4-(2-cyanovinyl)-6-methylphenoxy)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(2-chloro-4-(2-cyanovinyl)-6-methylphenoxy)-5-methyl-7-oxo-6,7-dihydro-5H-pyrrolo[3,4-d]pyrimidin-2-yl)amino)bicyclo[1.1.1]pentane-1-carbonitrile

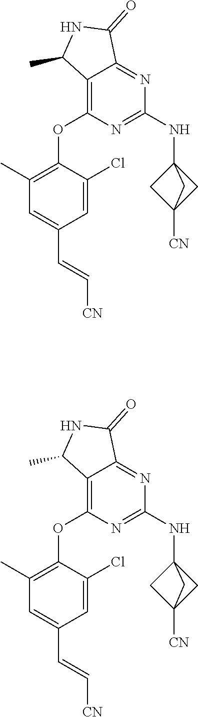 [SMILES: Cc1cc(/C=C/C#N)cc(Cl)c1Oc1nc(NC23CC(C#N)(C2)C3)nc2c1[C@@H](C)NC2=O ; Cc1cc(/C=C/C#N)cc(Cl)c1Oc1nc(NC23CC(C#N)(C2)C3)nc2c1[C@H](C)NC2=O]

### Compounds 157a and 157b: (R,E)-3-((4-(2-chloro-4-(2-cyanovinyl)-6-ethylphenoxy)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(2-chloro-4-(2-cyanovinyl)-6-ethylphenoxy)-5-methyl-7-oxo-6,7-dihydro-5H-pyrrolo[3,4-d]pyrimidin-2-_yl)amino)bicyclo[1.1.1]pentane-1-carbonitrile

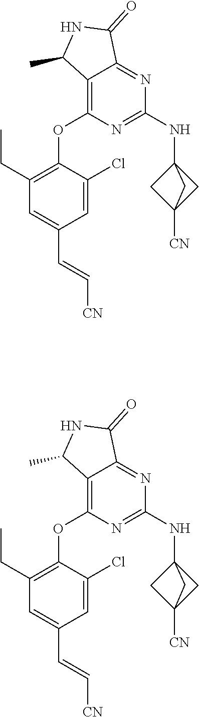 [SMILES: CCc1cc(/C=C/C#N)cc(Cl)c1Oc1nc(NC23CC(C#N)(C2)C3)nc2c1[C@@H](C)NC2=O ; CCc1cc(/C=C/C#N)cc(Cl)c1Oc1nc(NC23CC(C#N)(C2)C3)nc2c1[C@H](C)NC2=O]

### Compounds 158a and 158b: (R,E)-3-((4-(2-chloro-4-(2-cyanovinyl)-6-methoxyphenoxy)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(2-chloro-4-(2-cyanovinyl)-6-methoxyphenoxy)-5-methyl-7-oxo-6,7-dihydro-5H-pyrrolo[3,4-d]pyrimidin-2-yl)amino)bicyclo[1.1.1]pentane-1-carbonitrile

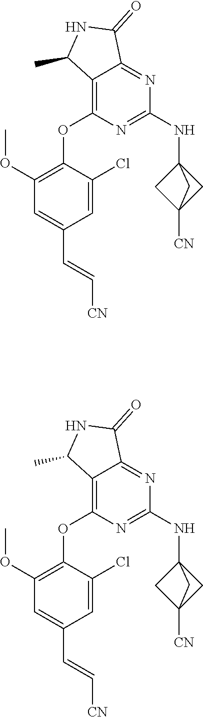 [SMILES: COc1cc(/C=C/C#N)cc(Cl)c1Oc1nc(NC23CC(C#N)(C2)C3)nc2c1[C@@H](C)NC2=O ; COc1cc(/C=C/C#N)cc(Cl)c1Oc1nc(NC23CC(C#N)(C2)C3)nc2c1[C@H](C)NC2=O]

### Compounds 159a and 159b: (R,E)-3-((4-(2-chloro-4-(2-cyanovinyl)-6-ethoxyphenoxy)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(2-chloro-4-(2-cyanovinyl)-6-ethoxyphenoxy)-5-methyl-7-oxo-6,7-dihydro-5H-pyrrolo[3,4-d]pyrimidin-2-yl)amino)bicyclo[1.1.1]pentane-1-carbonitrile

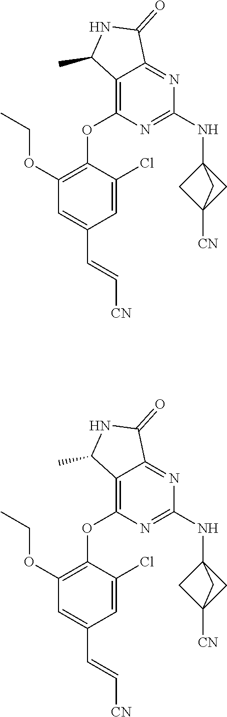 [SMILES: CCOc1cc(/C=C/C#N)cc(Cl)c1Oc1nc(NC23CC(C#N)(C2)C3)nc2c1[C@@H](C)NC2=O ; CCOc1cc(/C=C/C#N)cc(Cl)c1Oc1nc(NC23CC(C#N)(C2)C3)nc2c1[C@H](C)NC2=O]

### Compounds 160a and 160b: (R,E)-3-((4-(2-chloro-4-(2-cyanovinyl)-6-fluorophenoxy)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(2-chloro-4-(2-cyanovinyl)-6-fluorophenoxy)-5-methyl-7-oxo-6,7-dihydro-5H-pyrrolo[3,4-d]pyrimidin-2-yl)amino)bicyclo[1.1.1]pentane-1-carbonitrile

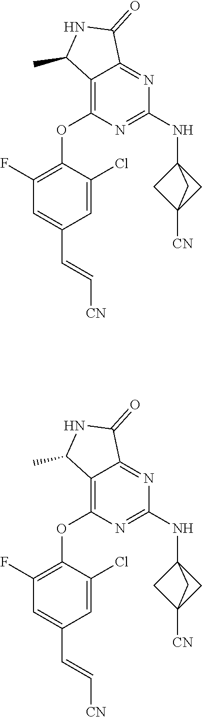 [SMILES: C[C@H]1NC(=O)c2nc(NC34CC(C#N)(C3)C4)nc(Oc3c(F)cc(/C=C/C#N)cc3Cl)c21 ; C[C@@H]1NC(=O)c2nc(NC34CC(C#N)(C3)C4)nc(Oc3c(F)cc(/C=C/C#N)cc3Cl)c21]

### Compounds 161a and 161b: (R,E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(4-(2-cyanovinyl)-2,6-dimethylphenoxy)-6-cyclopropyl-5-methyl-7-oxo-6,7-dihydro-5H-pyrrolo[3,4-d]pyrimidin-2-yl) amino)bicyclo[1.1.1]pentane-1-carbonitrile

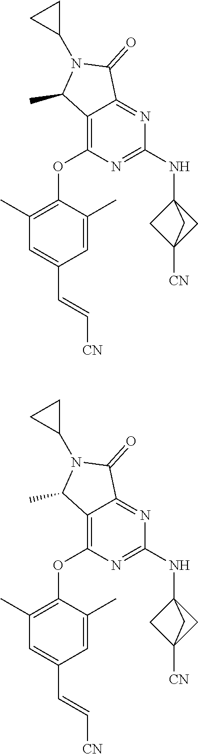 [SMILES: Cc1cc(/C=C/C#N)cc(C)c1Oc1nc(NC23CC(C#N)(C2)C3)nc2c1[C@@H](C)N(C1CC1)C2=O ; Cc1cc(/C=C/C#N)cc(C)c1Oc1nc(NC23CC(C#N)(C2)C3)nc2c1[C@H](C)N(C1CC1)C2=O]

### Compounds 162a and 162b: (R,E)-3-((4-(4-(2-cyanovinyl)-2,6-dimethylphenoxy)-6-(2,2-difluoroethyl)-5-methyl-7-oxo-6,7-dihydro-5H-pyrrolo[3,4-d]pyrimidin-2-yl)amino)bicyclo[1.1.1]pentane-1-carbonitrile and (S,E)-3-((4-(4-(2-cyanovinyl)-2,6-dimethylphenoxy)-6-(2,2-difluoroethyl)-5-methyl-7-oxo-6,7-dihydro-5H-pyrrolo[3,4-d]pyrimidin-2-yl)amino)bicyclo[1.1.1]pentane-1-carbonitrile

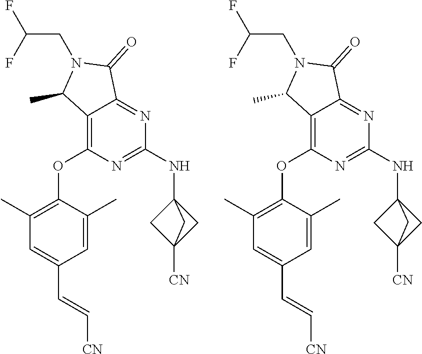

### Compound 163: (E)-3-((4-(4-(2-cyanovinyl)-2,6-dimethylphenoxy)-6-cyclopropyl-5,5-dimethyl-7-oxo-6,7-dihydro-5H-pyrrolo[3,4-d]pyrimidin-2-yl)amino)bicyclo[1.1.1]pentane-1-carbonitrile

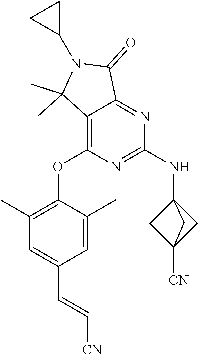 [SMILES: Cc1cc(/C=C/C#N)cc(C)c1Oc1nc(NC23CC(C#N)(C2)C3)nc2c1C(C)(C)N(C1CC1)C2=O]

### Compound 164: (E)-3-((4-(4-(2-cyanovinyl)-2,6-dimethylphenoxy)-6-(2,2-difluoroethyl)-5,5-dimethyl-7-oxo-6,7-dihydro-5H-pyrrolo[3,4-d]pyrimidin-2-yl)amino)bicyclo[1.1.1]pentane-1-carbonitrile

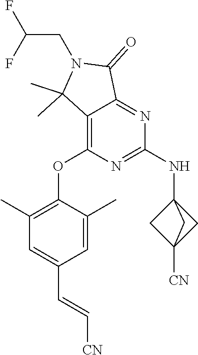 [SMILES: Cc1cc(/C=C/C#N)cc(C)c1Oc1nc(NC23CC(C#N)(C2)C3)nc2c1C(C)(C)N(CC(F)F)C2=O]

### Compound 165: (E)-3-((4-(4-(2-cyanovinyl)-2,6-dimethylphenoxy)-5,5-dimethyl-7-oxo-6,7-dihydro-5H-pyrrolo[3,4-d]pyrimidin-2-yl)amino)bicyclo[1.1.1]pentane-1-carbonitrile

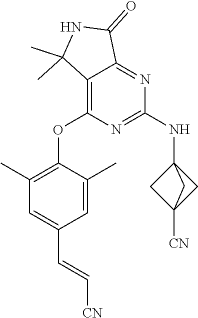 [SMILES: Cc1cc(/C=C/C#N)cc(C)c1Oc1nc(NC23CC(C#N)(C2)C3)nc2c1C(C)(C)NC2=O]

### Compounds 166a and 166b: (S,E)-3-((4-(4-(2-cyanovinyl)-2,6-dimethylphenoxy)-5-hydroxy-5-methyl-7-oxo-6,7-dihydro-5H-pyrrolo[3,4-d]pyrimidin-2-yl)amino)bicyclo[1.1.1]pentane-1-carbonitrile and (R,E)-3-((4-(4-(2-cyanovinyl)-2,6-dimethylphenoxy)-5-hydroxy-5-methyl-7-oxo-6,7-dihydro-5H-pyrrolo[3,4-d]pyrimidin-2-yl)amino)bicyclo[1.1.1]pentane-1-carbonitrile

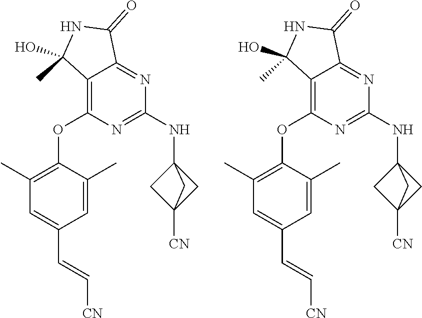

### Compounds 167a and 167b: (S,E)-3-((4-(4-(2-cyanovinyl)-2,6-dimethylphenoxy)-5-(difluoromethyl)-5-hydroxy-7-oxo-6,7-dihydro-5H-pyrrolo[3,4-d]pyrimidin-2-yl)amino)bicyclo[1.1.1]pentane-1-carbonitrile and (R,E)-3-((4-(4-(2-cyanovinyl)-2,6-dimethylphenoxy)-5-(difluoromethyl)-5-hydroxy-7-oxo-6,7-dihydro-5H-pyrrolo[3,4-d]pyrimidin-2-yl)amino)bicyclo[1.1.1]pentane-1-carbonitrile

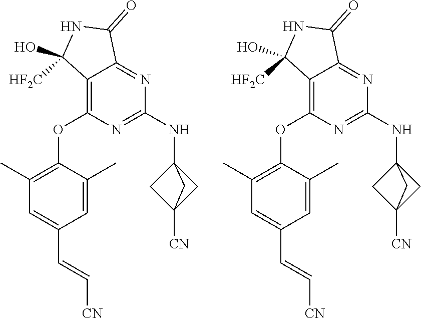

### Compounds 168a and 168b: (R,E)-3-((4-(4-(2-cyanovinyl)-2,6-dimethylphenoxy)-5-cyclopropyl-7-oxo-6,7-dihydro-5H-pyrrolo[3,4-d]pyrimidin-2-yl)amino)bicyclo[1.1.1]pentane-1-carbonitrile and (S,E)-3-((4-(4-(2-cyanovinyl)-2,6-dimethylphenoxy)-5-cyclopropyl-7-oxo-6,7-dihydro-5H-pyrrolo[3,4-d]pyrimidin-2-yl)amino)bicyclo[1.1.1]pentane-1-carbonitrile

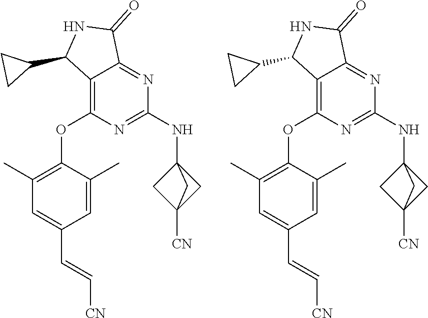

### Compounds 169a and 169b: (S,E)-3-((4-(4-(2-cyanovinyl)-2,6-dimethylphenoxy)-5-(difluoromethyl)-7-oxo-6,7-dihydro-5H-pyrrolo[3,4-d]pyrimidin-2-yl)amino)bicyclo[1.1.1]pentane-1-carbonitrile and (R,E)-3-((4-(4-(2-cyanovinyl)-2,6-dimethylphenoxy)-5-(difluoromethyl)-7-oxo-6,7-dihydro-5H-pyrrolo[3,4-d]pyrimidin-2-yl)amino)bicyclo[1.1.1]pentane-1-carbonitrile

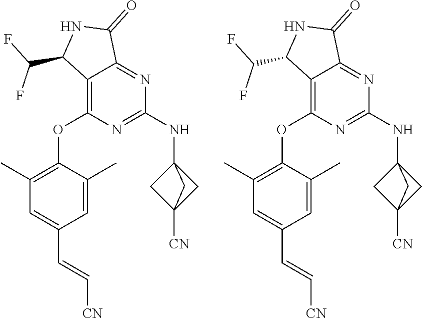

### Compounds 170a and 170b: (S,E)-3-((4-(4-(2-cyanovinyl)-2,6-dimethylphenoxy)-7-oxo-5-(trifluoromethyl)-6,7-dihydro-5H-pyrrolo[3,4-d]pyrimidin-2-yl)amino)bicyclo[1.1.1]pentane-1-carbonitrile and (R,E)-3-((4-(4-(2-cyanovinyl)-2,6-dimethylphenoxy)-7-oxo-5-(trifluoromethyl)-6,7-dihydro-5H-pyrrolo[3,4-d]pyrimidin-2-yl)amino)bicyclo[1.1.1]pentane-1-carbonitrile

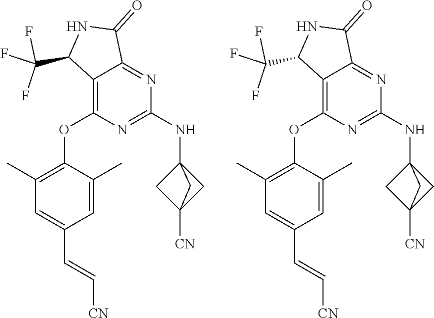

### Compound 171: (E)-3-((6-(4-(2-cyanovinyl)-2,6-dimethylphenoxy)-8-ethoxy-7-methyl-7H-purin-2-yl)amino)bicyclo[1.1.1]pentane-1-carbonitrile

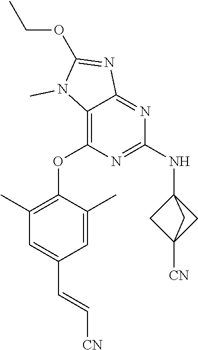 [SMILES: CCOc1nc2nc(NC34CC(C#N)(C3)C4)nc(Oc3c(C)cc(/C=C/C#N)cc3C)c2n1C]

### Compound 172: (E)-3-((6-(4-(2-cyanovinyl)-2,6-dimethylphenoxy)-7-(difluoromethyl)-8-ethoxy-7H-purin-2-yl)amino)bicyclo[1.1.1]pentane-1-carbonitrile

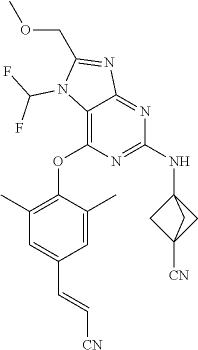 [SMILES: COCc1nc2nc(NC34CC(C#N)(C3)C4)nc(Oc3c(C)cc(/C=C/C#N)cc3C)c2n1C(F)F]

### Compound 173: (E)-3-((6-(4-(2-cyanovinyl)-2,6-dimethylphenoxy)-7-(difluoromethyl)-8-(hydroxymethyl)-7H-purin-2-yl)amino)bicyclo[1.1.1]pentane-1-carbonitrile

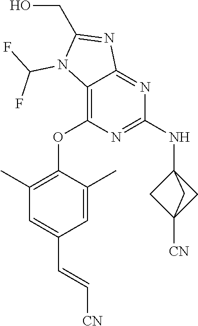 [SMILES: Cc1cc(/C=C/C#N)cc(C)c1Oc1nc(NC23CC(C#N)(C2)C3)nc2nc(CO)n(C(F)F)c12]

### Compounds 174a and 174b: (S,E)-3-((6-(4-(2-cyanovinyl)-2,6-dimethylphenoxy)-7-(difluoromethyl)-8-(1-hydroxyethyl)-7H-purin-2-yl)amino)bicyclo[1.1.1]pentane-1-carbonitrile and (R,E)-3-((6-(4-(2-cyanovinyl)-2,6-dimethylphenoxy)-7-(difluoromethyl)-8-(1-hydroxyethyl)-7H-purin-2-yl)amino)bicyclo[1.1.1]pentane-1-carbonitrile

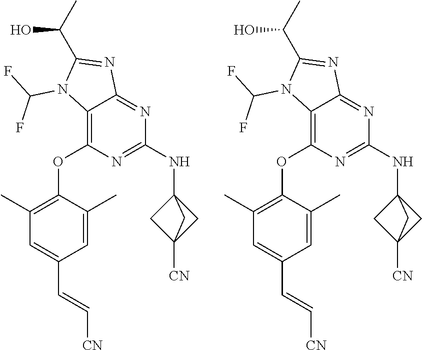

### Compound 175: (E)-3-((6-(4-(2-cyanovinyl)-2,6-dimethylphenoxy)-7-(difluoromethyl)-8-(methoxymethyl)-7H-purin-2-yl)amino)bicyclo[1.1.1]pentane-1-carbonitrile

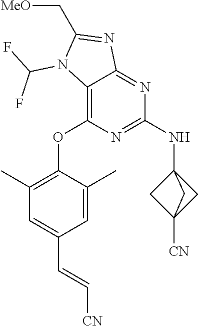 [SMILES: COCc1nc2nc(NC34CC(C#N)(C3)C4)nc(Oc3c(C)cc(/C=C/C#N)cc3C)c2n1C(F)F]

### Assays

Compounds were screened in a miniaturized, high throughput cell killing assay using monocytic THP1 cells infected with HIV VSV-G pseudotyped GFP-NEF reporter virus. The assay measures the ability of compounds to induce pyroptosis by detecting the reduction in the level of HIV p24 protein produced by infected cells treated with compound using a sandwich immunoassay. THP1 cells were maintained in cell culture media (RPMI supplemented with 10% fetal bovine serum and 1% penicillin-streptomycin-glutamine). Ten-point serial dilutions of compounds with half-log step size were generated in DMSO. Reference Compound 1 (1 uM, disclosed as Example 25 in WO 2020236692) was used as the positive control and 100% DMSO as the negative control. The Echo acoustic dispenser was used to deliver 50 nL of serially diluted compound into sterile 384 well tissue culture assay plates. Briefly, THP1 cells were infected with HIV GFP reporter virus via spinfection for 2 hours. After 2 hours, cells were washed with cell culture media and placed back in the flask for 72 hours incubation in a 37° C., 5% CO₂ incubator. After 72 hours, cells were activated with LPS (0.1 ug/mL) for 2 hours and then harvested and resuspended at 1 million cells/mL using cell culture media. 10 μL of infected cells were dispensed onto assay plates containing serially diluted compounds and controls. Assay plates were placed in a 37° C., 5% CO₂ incubator for 24 hours. After 24 hours, 5 μL of HTRF anti-HIV p24 detection antibodies and lysis buffer were added to all wells. (HTRF kit from Cisbio, cat #64P24PEH). Assay plates were centrifuged and incubated at room temperature for 2 hours and then read on an Envision plates reader using HTRF reader settings. Data were normalized to positive and negative controls in each plate and expressed as % Inhibition. The EC₅₀ values are shown in Table 1 and defined as the compound concentration that caused a 50% decrease in HTRF signal, calculated by non-linear regression using a four parameter fit equation.

TABLE 1

| Compound | EC₅₀ (M) |
|---|---|
| 1 | 46.655 |
| 2 | 137.75 |
| 3 | 29.602 |
| 4 | 1494.8 |
| 5 | 26.078 |
| 6 | 17.379 |
| 7 | 21.369 |
| 8 | 44.65 |
| 10 | 65.945 |
| 11 | 27.668 |
| 12 | 5.68 |
| 13 | 11.659 |
| 14 | 81.347 |
| 15 | 108.54 |
| 17 | 21.411 |
| 18 | 23.997 |
| 19 | 90.915 |
| 20 | 25.518 |
| 21 | 280.41 |
| 22 | 258.82 |
| 23 | 31.749 |
| 24 | 32.041 |
| 25 | 37.151 |
| 26 | 347.3 |
| 27 | 68.302 |
| 28 | 43.962 |
| 42 | 518.42 |
| 43 | 90.458 |
| 45 | 53.029 |
| 46 | 88.619 |
| 47 | 117.63 |
| 48 | 150.64 |
| 49 | 110.49 |
| 50 | 165.64 |
| 51 | 374.18 |
| 52 | 136.56 |
| 53 | 232.93 |
| 54 | 364 |
| 55 | 129.68 |
| 56 | 142.1 |
| 57 | 147.11 |
| 60 | 372.7 |
| 62 | 161.19 |
| 63 | 62.827 |
| 64 | 122.53 |
| 65 | 131.81 |
| 66 | 297.97 |
| 67 | 230.91 |
| 68 | 272.83 |
| 69 | 495.81 |
| 70 | 415.36 |
| 71 | 274.26 |
| 72 | 308.34 |
| 73 | 284.93 |
| 74 | 162.31 |
| 76 | 25.275 |
| 79 | 33.244 |
| 80 | 592.71 |
| 81 | 35.625 |
| 82 | 78.966 |
| 83 | 162.49 |
| 84 | 369.94 |
| 86 | 18.344 |
| 87 | 25.051 |
| 88 | 23.36 |
| 89 | 24.426 |
| 90 | 25.352 |
| 91 | 42.315 |
| 92 | 26.317 |
| 93 | 37.678 |
| 94 | 72.48 |
| 95 | 30.647 |
| 96 | 360.49 |
| 97 | 32.541 |
| 98 | 94.293 |
| 99 | 37.058 |
| 100 | 36.087 |
| 101 | 59.565 |
| 102 | 22.369 |
| 103 | 220.62 |
| 104 | 140.75 |
| 105 | 181.84 |
| 106 | 215.84 |
| 107 | 443.04 |
| 108 | 611.29 |
| 109 | 200.26 |
| 110 | >1000 |
| 111 | 30.563 |
| 112 | 745.21 |
| 113 | 168.43 |
| 115 | 99.373 |
| 119 | 19.275 |
| 120 | 21.15 |
| 121 | 53.213 |
| 122 | 53.965 |
| 123 | 59.762 |
| 124 | 61.681 |
| 125 | 110.69 |
| 126 | 118.68 |
| 127 | 148.65 |
| 128 | 131.31 |
| 129 | 168.48 |
| 130 | 220.53 |
| 131 | 1472.9 |
| 132 | 266.7 |
| 133 | 510.97 |
| 134 | 262.24 |
| 135 | 274.51 |
| 136 | 15.03 |
| 137 | 6.898 |
| 138 | 18.331 |
| 139 | 8.272 |
| 140 | 14.339 |
| 141 | 73.017 |
| 142 | 23.985 |
| 143 | 58.545 |
| 144 | 81.039 |
| 145 | 89.117 |
| 146 | 35.383 |
| 148 | 48.077 |
| 150 | 99.152 |
| 154 | 235.48 |
| 153 | 27.53 |
| 176 | 45.021 |
| 177 | 45.29 |
| 178 | 25.724 |
| 179 | 18.145 |
| 180 | 92.932 |
| 181 | 20.456 |
| 182 | 34.156 |
| 183 | 80.034 |
| 184 | 36.536 |
| 185 | 22.29 |
| 186 | 92.025 |
| 187 | 36.452 |
| 188 | 70.364 |
| 189 | 109.38 |
| 190 | 329.19 |
| 191 | 51.761 |
| 192 | 27.107 |
| 193 | 318.88 |
| 194 | 49.696 |
| 195 | 37.001 |
| 196 | 24.622 |
| 197 | 97.321 |
| 198 | 115.25 |
| 199 | 48.956 |
| 200 | 174.65 |
| 201 | 88.607 |
| 202 | 18.929 |
| 203 | 27.531 |
| 204 | 23.399 |
| 205 | 42.726 |
| 206 | 279.96 |
| 207 | 70.346 |
| 208 | 28.764 |
| 209 | 57.745 |
| 210 | 125 |
| 211 | 54.459 |
| 212 | 47.142 |
| 213 | 43.851 |
| 214 | 59.126 |
| 215 | 108.97 |
| 216 | 77.777 |
| 217 | 157.34 |
| 218 | 47.29 |
| 219 | 40.733 |
| 220 | 155.46 |
| 221 | 72.921 |
| 222 | 70.737 |
| 223 | 38.297 |
| 224 | 51.143 |
| 225 | 51.761 |
| 226 | 84.357 |
| 227 | 114.93 |
| 228 | 71.972 |
| 229 | 122.59 |
| 230 | 115.69 |
| 231 | 31.139 |
| 232 | 109.6 |
| 233 | 157.51 |
| 234 | 331.76 |
| 235 | 22.797 |
| 236 | 217.07 |
| 237 | 119.27 |
| 238 | 92.434 |
| 239 | 18.544 |
| 240 | 35.239 |
| 241 | 34.797 |
| 242 | 36.4 |
| 243 | 219.45 |
| 244 | 110.93 |
| 245 | 22.333 |
| 246 | 41.217 |
| 247 | 40.206 |
| 248 | 121.34 |
| 249 | 75.686 |
| 250 | 64.02 |
| 251 | 73.213 |
| 252 | 77.147 |
| 253 | 253.23 |
| 254 | 71.364 |
| 255 | 52.512 |
| 256 | 36.052 |
| 257 | 22.826 |
| 258 | 177.76 |
| 259 | 23.648 |
| 263 | 60.407 |
| 264 | 118.35 |
| 265 | 86.369 |
| 266 | 61.01 |
| 267 | 80.336 |
| 268 | 173.49 |
| 269 | 57.296 |
| 270 | 18.994 |
| 271 | 51.995 |
| 272 | 68.34 |
| 273 | 15.967 |
| 274 | 111.18 |
| 275 | 113.48 |
| 276 | 194.21 |
| 277 | 49.934 |
| 278 | 30.803 |
| 279 | 57.225 |
| 280 | 33.686 |
| 281 | 74.211 |
| 282 | 50.06 |
| 283 | 44.815 |
| 284 | 34.896 |
| 285 | 92.488 |
| 286 | 72.439 |
| 287 | 26.187 |
| 288 | 29.842 |
| 289 | 52.92 |
| 290 | 386.79 |
| 291 | 31.097 |
| 292 | 58.79 |
| 293 | 56.285 |
| 294 | 23.314 |
| 295 | 55.348 |
| 296 | 59.532 |
| 297 | 159.31 |
| 298 | 87.304 |
| 299 | 40.139 |
| 300 | 87.604 |
| 301 | 50.705 |
| 302 | 210.51 |
| 303 | 232.16 |
| 304 | 106.97 |
| 305 | 45.011 |
| 308 | 60.062 |
| 312 | 15.093 |
| 313 | 467.9 |
| 314 | 25.292 |
| 315 | 187.6 |
| 316 | 77.48 |

Compounds were screened in a miniaturized, high throughput cell killing assay using CD4 cells, isolated from healthy donor PMBCs, and infected with HIV VSV-G pseudotyped GFP-GAG reporter virus. The assay measures the ability of compounds to induce pyroptosis by detecting the reduction in the level of HIV p24 protein produced by infected cells treated with compound using a sandwich immunoassay. Human CD4 cells were isolated from fresh PBMCs and activated with PHA/IL-2 (1:1000) and maintained in cell culture media (RPMI supplemented with 10% fetal bovine serum and 1% penicillin-streptomycin-glutamine) for 3 days in a 37° C., 5% CO2 incubator. Ten-point serial dilutions of compounds with half-log step size were generated in DMSO. Reference Compound 1 (1 uM, disclosed as Example 25 in WO 2020236692) was used as the positive control and 100% DMSO as the negative control. The Echo acoustic dispenser was used to deliver 100 nL of serially diluted compound into sterile 384 well tissue culture assay plates. Briefly, activated CD4 cells were infected with HIV GFP reporter virus via nutation for 4 hours. After 4 hours, cells were washed with cell culture media and placed back in the flask for 24 hours incubation in a 37° C., 5% CO₂ incubator. After 24 hours, cells were harvested and resuspended at 1 million cells/mL using cell culture media. 20 μL of infected cells were dispensed onto assay plates containing serially diluted compounds and controls. Assay plates were placed in a 37° C., 5% CO₂ incubator for 72 hours. After 72 hours, 2 μL of lysis buffer and 5 μL of HTRF anti-HIV p24 detection antibodies were added to all wells. (HTRF kit from Cisbio, cat #64P24PEH). Assay plates were centrifuged and incubated at room temperature for 2 hours and then read on an Envision plates reader using HTRF reader settings. Data were normalized to positive and negative controls in each plate and expressed as % Inhibition. EC50 values are shown in Table 2 were defined as the compound concentration that caused a 50% decrease in HTRF signal and were calculated by non-linear regression using a four parameter fit equation.

TABLE 2

| Compound | EC₅₀ (M) |
|---|---|
| 10 | 54.713 |
| 14 | 84.153 |
| 23 | 17.018 |
| 27 | 99.376 |
| 41 | 77.593 |
| 42 | 893.76 |
| 43 | 87.74 |
| 44 | 108.66 |
| 49 | 166.25 |
| 50 | 150.55 |
| 55 | 25.889 |
| 56 | 96.606 |
| 58 | 429.52 |
| 59 | 336.12 |
| 60 | 174.82 |
| 64 | 274.46 |
| 75 | 55.035 |
| 76 | 19.348 |
| 77 | 28.177 |
| 78 | 12.102 |
| 80 | 550.77 |
| 85 | 20.871 |
| 89 | 16.599 |
| 97 | 51.13 |
| 106 | 236.83 |
| 112 | 294.37 |
| 113 | 101.6 |
| 114 | 51.111 |
| 115 | 111.13 |
| 116 | 63.336 |
| 117 | 23.88 |
| 118 | 80.358 |
| 119 | 16.901 |
| 120 | 22.866 |
| 122 | 47.33 |
| 124 | 27.032 |
| 128 | 106.15 |
| 129 | 131.21 |
| 130 | 56.026 |
| 135 | 39.482 |
| 142 | 17.608 |
| 148 | 40.995 |
| 149 | 492.11 |
| 150 | 110.22 |
| 151 | 76.044 |
| 152 | 3365 |
| 155 | 418.79 |
| 154 | 323.64 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter, and is not intended to limit the appended claims to the specific embodiments illustrated.

1. A compound of Formula (I) 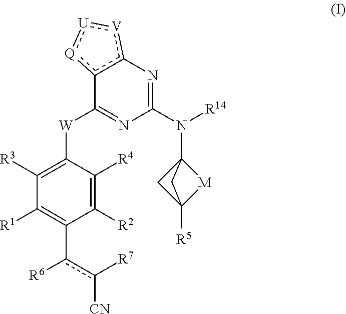
or a pharmaceutically acceptable salt thereof, wherein: [symbol]
is a single bond or a double bond;
R¹ is selected from H, CN, halogen, C₁₋₃alkyl, C₁₋₃haloalkyl, —OR^{1A}, —SR^{1A}, —NR^{1A} and —N(R^{1A})₂;
R² is selected from H, CN, halogen, C₁₋₃alkyl, C₁₋₃haloalkyl, —OR^{2A}, —SR^{2A}, —NHR^{2A} and —N(R^{2A})₂;
R³ is selected from CN, halogen, C₁₋₆alkyl, C₁₋₆haloalkyl, —OR^{3A}, —CH₂OR^{3A}, —SR^{3A}, —NHR^{3A}, —N(R^{3A})₂, C₃₋₇cycloalkyl, and 3-7 membered heterocycloalkyl;
R⁴ is selected from CN, halogen, C₁₋₆alkyl, C₁₋₆haloalkyl, —OR^{4A}, —CH₂OR^{4A}, —SR^{4A}, —NHR^{4A}, —N(R^{4A})₂, C₃₋₇cycloalkyl, and 3-7 membered heterocycloalkyl;
or R¹ and R³ together form a 6-10 membered aryl;
R⁵ is selected from H, CN, halogen, C₁₋₆alkyl, C₁₋₆haloalkyl, C₂₋₆alkenyl, C₂₋₆alkynyl, —NHR^{5A}, —N(R^{5A})₂, —OR^{5A}, C₃₋₇cycloalkyl, 3-10 membered heterocycloalkyl, —(C═O)NHR^{5A}, —(C═O)N(R^{5A})₂, and —(C═O)OR^{5A}; wherein the C₁₋₆alkyl, C₁₋₆haloalkyl, C₂₋₆alkenyl, C₂₋₆alkynyl, C₃₋₇cycloalkyl, and 3-10 membered heterocycloalkyl of R⁵ are each optionally substituted with 1 or 2 R⁸ groups;
R⁶ and R⁷ are independently selected from H and C₁₋₆alkyl; or R⁶ and R⁷ together form a C₃₋₇cycloalkyl or 3-7 membered heterocycloalkyl;
each R⁸ is independently selected from halogen, CN, C₁₋₃alkyl, C₁₋₃haloalkyl, C₁₋₃alkoxyl, C₃₋₇cycloalkyl, and 3-7 membered heterocycloalkyl;
M is selected from —CH₂—, —CF₂—, —CH₂CH₂—, and —CH₂O—;
W is selected from —O—, —S—, —C(R^{W})₂—, —NR^{W}—, and —C(═O)—;
each R^{W} is independently selected from H, —OR^{W1}, C₁₋₆alkyl, C₃₋₇cycloalkyl, C₁₋₆haloalkyl, and —(C═O)OR^{W1};
Q, U, and V are independently selected from N, O, S, CR^{Q}, C(R^{Q})₂, NR^{V}, N(R^{V})₂⁺, S═O, SO₂, C═CH₂, C═CHF, and C═O;
each R^{Q} is independently selected from H, halogen, —OR^{Q2}, CN, C₁₋₆alkyl, C₁₋₆haloalkyl, C₂₋₆alkenyl, C₂₋₆alkynyl, C₃₋₇cycloalkyl, 3-7 membered heterocycloalkyl, —NHR^{Q2}, —N(R^{Q2})₂, —(C=O)OR^{Q1}, —(C═O)NHR^{Q2}, —(C═O)N(R^{Q2})₂, —NH(C═O)R^{Q1}, —NH(C═O)OR^{Q1}, —NH(C=O)NHR^{Q2}, —NH(C═O)N(R^{Q2})₂, 6-10 membered aryl, and 5-10 membered heteroaryl; wherein each C₁₋₆alkyl, C₁₋₆haloalkyl, C₂₋₆alkenyl, C₂₋₆alkynyl, C₃₋₇cycloalkyl, 3-7 membered heterocycloalkyl, 6-10 membered aryl, and 5-10 membered heteroaryl of R^{Q} are each optionally substituted with 1, 2, 3, or 4 R⁹ groups;
each R^{V} is independently selected from H, C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, 5-10 membered heteroaryl, —(C═O)R^{V1}, —(C═O)OR^{V1}, —(C═O)NHR^{V1}, —(C═O)N(R^{V1})₂, —S(O)R^{V1}, and —S(O)₂R^{V1}; wherein each C₁₋₆alkyl, C₁₋₆haloalkyl, C₂₋₆alkenyl, C₂₋₆alkynyl, C₃₋₇cycloalkyl, 4-7 membered heterocycloalkyl, 6-10 membered aryl, and 5-10 membered heteroaryl of R^{V} are each optionally substituted with 1, 2, 3, or 4 R¹⁰ groups;
each R^{Q2} is independently selected from H, CN, C₁₋₆alkyl, C₁₋₆haloalkyl, C₁₋₆alkoxy, C₃₋₇ cycloalkyl, 4-7 membered heterocycloalkyl, 6-10 membered aryl, 5-10 membered heteroaryl, —(C=O)R^{X1}, —(C═O)OR^{X1}, —(C═O)NHR^{X1}, —(C═O)N(R^{X1})₂, and —(SO₂)R^{X1}; wherein each C₃₋₇ cycloalkyl, 4-7 membered heterocycloalkyl, 6-10 membered aryl, and 5-10 membered heteroaryl of R^{Q2} is optionally substituted with 1, 2, 3 or 4 R¹¹ groups;
or two R^{Q2} together form a 4-7 membered heterocycloalkyl;
or two R^{V} together form a 4-7 membered heterocycloalkyl;
or R^{Q} and R^{V} together form a 4-11 membered heterocycloalkyl or 5-10 membered heteroaryl, wherein the 4-11 membered heterocycloalkyl or 5-10 membered heteroaryl is optionally substituted with 1, 2, 3 or 4 R¹³ groups;
each R⁹ and R¹⁰ are independently selected from halogen, —OR^{X2}, CN, C₁₋₆alkyl, C₁₋₆haloalkyl, C₁₋₆alkoxy, C₃₋₇cycloalkyl, 3-7 membered heterocycloalkyl, 6-10 membered aryl, 5-10 membered heteroaryl, —(C═O)R^{X2}, —(C═O)OR^{X2}, —(C═O)NR^{X2}, —(C═O)N(R^{X2})₂, and —(SO₂)R^{X2}; wherein each C₃₋₇cycloalkyl, 3-7 membered heterocycloalkyl, 6-10 membered aryl, and 5-10 membered heteroaryl of R⁹ and R¹⁰ are each optionally substituted with 1, 2, 3 or 4 R¹² groups;
each R¹¹, R¹² and R¹³ are independently selected from halogen, —OR^{X3}, CN, C₁₋₆alkyl, C₁₋₆ haloalkyl, C₁₋₆alkoxyl, —NHR^{X3}, —N(R^{X3})₂, C₃₋₇cycloalkyl, and 3-7 membered heterocycloalkyl; wherein each C₁₋₆alkyl of R¹¹, R¹² and R¹³ is optionally substituted with —OR^{X4};
R¹⁴ is selected from H and C₁₋₆alkyl; and
each R^{1A}, R^{2A}, R^{3A}, R^{4A}, R^{5A}, R^{Q1}, R^{V1}, R^{W1}, R^{X1}, R^{X2}, R^{X3}, and R^{X4} are independently selected from H, C₁₋₆alkyl, C₁₋₆haloalkyl, C₃₋₇cycloalkyl, and 6-10 membered aryl.

2-6. (canceled)

7. The compound of claim 1, or a pharmaceutically acceptable salt thereof, wherein R¹ is selected from H, C₁₋₃alkyl, and C₁₋₃haloalkyl.

8. (canceled)

9. The compound of claim 1, or a pharmaceutically acceptable salt thereof, wherein R² is selected from H, C₁₋₃alkyl, and C₁₋₃haloalkyl.

10-11. (canceled)

12. The compound of claim 1, or a pharmaceutically acceptable salt thereof, wherein R³ is selected from halogen, C₁₋₆alkyl, C₁₋₆haloalkyl, and —OR^{3A}.

13. (canceled)

14. The compound of claim 1, or a pharmaceutically acceptable salt thereof, wherein R⁴ is selected from halogen, C₁₋₆alkyl, C₁₋₆haloalkyl, and —OR^{4A}.

15-17. (canceled)

18. The compound of claim 1, or a pharmaceutically acceptable salt thereof, wherein R⁵ is selected from H, CN, C₁₋₆haloalkyl, and 3-10 membered heterocycloalkyl.

19. (canceled)

20. The compound of claim 1, or a pharmaceutically acceptable salt thereof, wherein R⁶ and R⁷ are independently selected from H and C₁₋₆alkyl; or R⁶ and R⁷ together form a C₃₋₇cycloalkyl.

21-23. (canceled)

24. The compound of claim 1, or a pharmaceutically acceptable salt thereof, wherein Q, U, and V are independently selected from N, O, S, CR^{Q}, C(R^{Q})₂, NR^{V}, N(R^{V})₂, S═O, C═CH₂, C═CHF, and C═O.

25-26. (canceled)

27. The compound of claim 1, or a pharmaceutically acceptable salt thereof, wherein each R^{Q} is independently selected from H, halogen, —OR^{Q2}, CN, C₁₋₆alkyl, C₁₋₆haloalkyl, C₂₋₆alkenyl, C₃₋₇cycloalkyl, 3-7 membered heterocycloalkyl, —NHR^{Q2}, and 5-10 membered heteroaryl; wherein each C₁₋₆alkyl, C₁₋₆haloalkyl, C₂₋₆alkenyl, C₃₋₇cycloalkyl, 3-7 membered heterocycloalkyl, and 5-10 membered heteroaryl of R^{Q} are each optionally substituted with 1 or 2 R⁹ groups.

28-30. (canceled)

31. The compound of claim 1, or a pharmaceutically acceptable salt thereof, wherein each R^{V} is independently selected from H, C₁₋₆alkyl, C₁₋₆haloalkyl, C₂₋₆alkenyl, C₃₋₇cycloalkyl, 4-7 membered heterocycloalkyl, 6-10 membered aryl, 5-10 membered heteroaryl, and —(C═O)OR^{V1}; wherein each C₁₋₆alkyl, C₁₋₆haloalkyl, C₃₋₇cycloalkyl, 4-7 membered heterocycloalkyl, and 6-10 membered aryl of R^{V} are each optionally substituted with 1, 2, or 3 R¹⁰ groups.

32-36. (canceled)

37. The compound of claim 1, or a pharmaceutically acceptable salt thereof, wherein each R⁹ and R¹⁰ are independently selected from halogen, —OR^{X2}, CN, C₁₋₆alkyl, C₁₋₆haloalkyl, C₃₋₇cycloalkyl, 3-7 membered heterocycloalkyl, 6-10 membered aryl, 5-10 membered heteroaryl, and —(C═O)NR^{X2}; wherein the C₃₋₇cycloalkyl, 6-10 membered aryl, and 5-10 membered heteroaryl of R⁹ and R¹⁰ are each optionally substituted with 1 or 2 R¹² groups.

38-44. (canceled)

45. The compound of claim 1, or a pharmaceutically acceptable salt thereof, wherein the compound is a compound of Formula selected from (IIb), (IIc), (IId), (IIe), (IIf), (IIg), (IIh), (IIi), and (IIj):

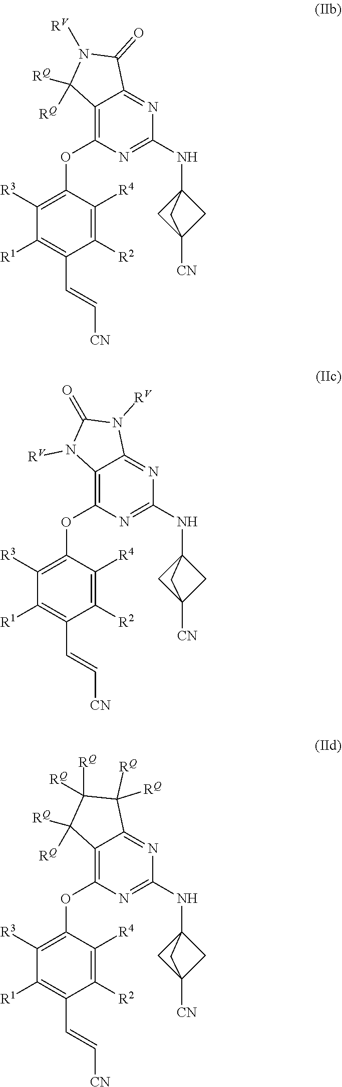

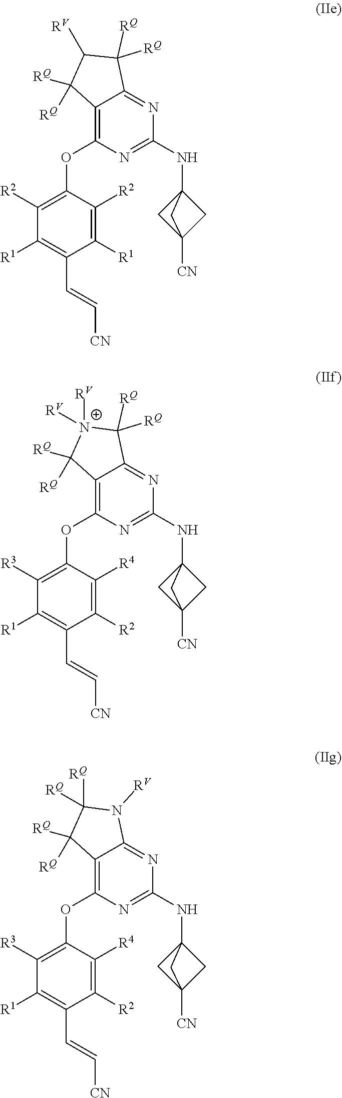

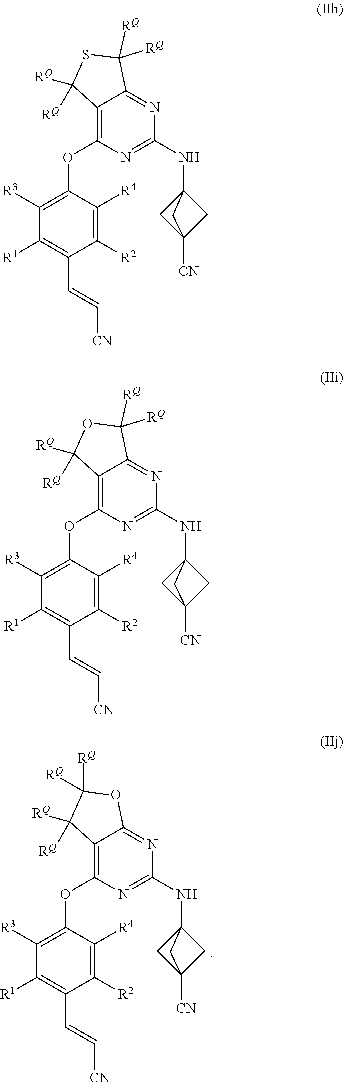

46. The compound of claim 1, or a pharmaceutically acceptable salt thereof, wherein the compound is a compound of Formula selected from (IIIb), (IIIc), (IIIcc), (IIId), (IIIe), (IIIf), (IIIg), (IIIh), (IIIi), (IIIj), (IIIk), (IIIl), (IIIm), and (IIIn).

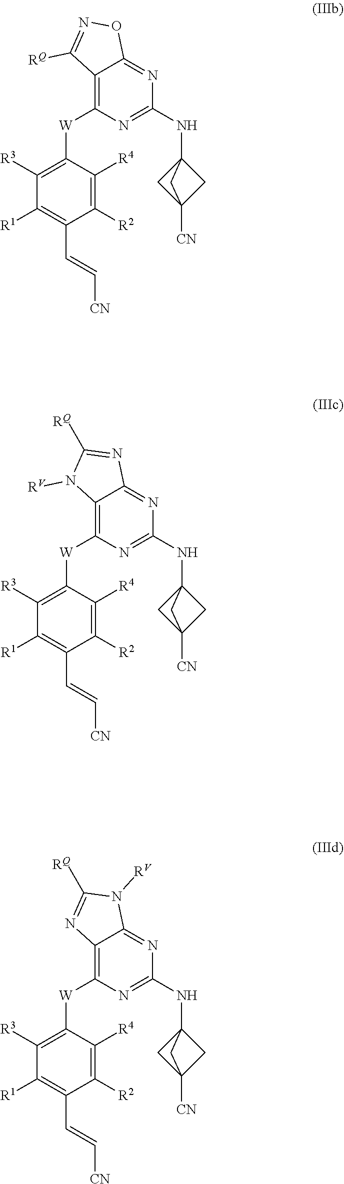

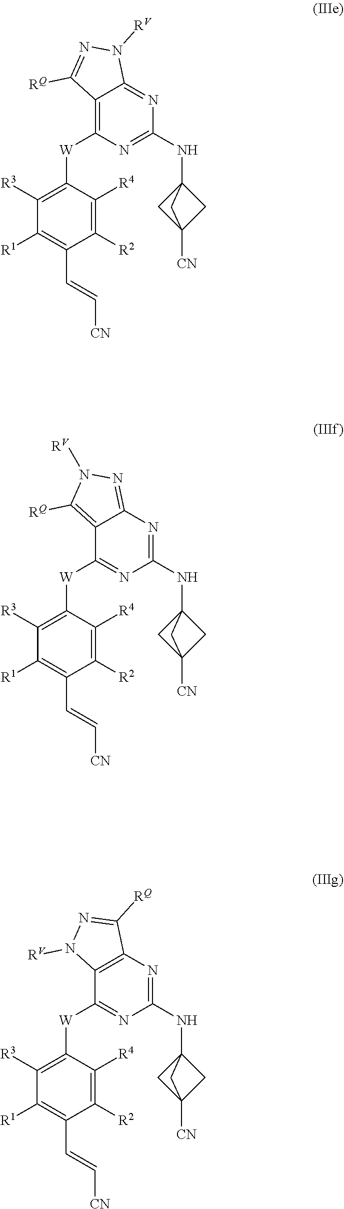

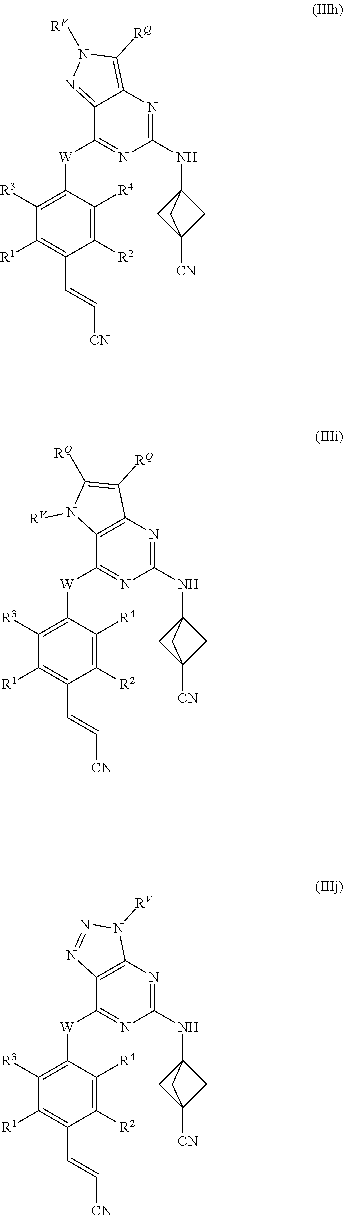

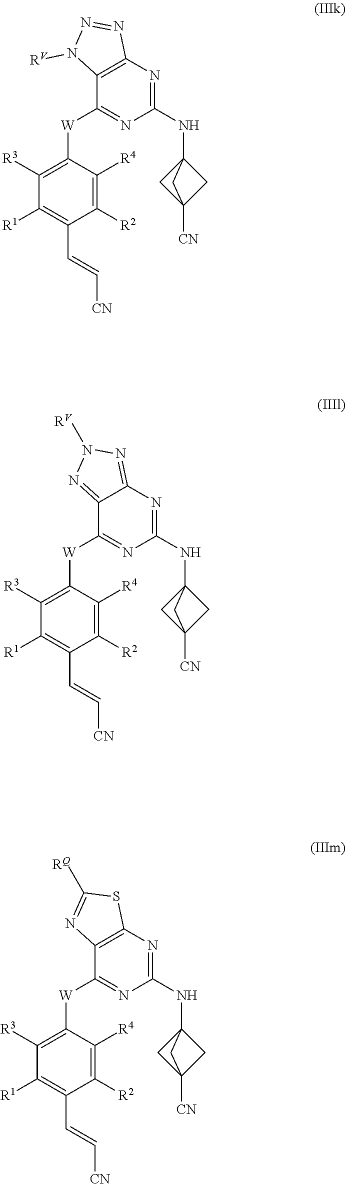

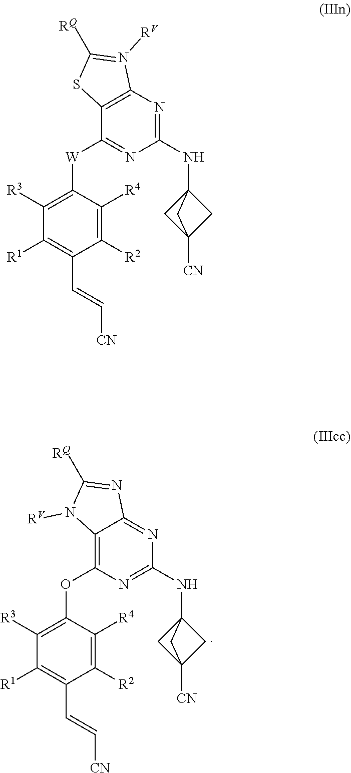

47. A compound selected from examples 1-155, or a pharmaceutically acceptable salt thereof.

48. A compound selected from examples 176-316, or a pharmaceutically acceptable salt thereof.

49. A pharmaceutical composition comprising a compound of claim 1, or a pharmaceutically acceptable salt thereof, and a pharmaceutically acceptable carrier.

50. The pharmaceutical composition of claim 49, further comprising at least one additional therapeutic agent.

51. (canceled)

52. A method of activating an HIV protease in a subject in need thereof, comprising administering a compound of claim 1, a pharmaceutically acceptable salt thereof, to the subject.

53. A method for treating or preventing an HIV infection in a subject comprising administering to the subject a compound of claim 1, or a pharmaceutically acceptable salt thereof.

54. A method for treating or preventing an HIV infection in a subject comprising administering to the subject in need thereof a compound of claim 1, or a pharmaceutically acceptable salt thereof, in combination with a therapeutically effective amount of one or more additional therapeutic agents selected from the group consisting of HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drugs for treating or preventing HIV, and combinations thereof.

55-60. (canceled)
